(19) United States
(12) Patent Application Publication
(10) Pub. No.: US 2009/0264428 A1
(43) Pub. Date: Oct. 22, 2009

(54) Renin Inhibitors

(76) Inventors: John J. Baldwin, Gwynedd Valley, PA (US); David A. Claremon, Maple Glen, PA (US); Colin M. Tice, Ambler, PA (US); Salvacion Cacatian, Blue Bell, PA (US); Lawrence W. Dillard, Yardley, PA (US); Alexey V. Ishchenko, Somerville, MA (US); Jing Yuan, Lansdale, PA (US); Zhenrong Xu, Horsham, PA (US); Gerard McGeehan, Garnet Valley, PA (US); Wei Zhao, Eagleville, PA (US); Robert D. Simpson, Wilmington, DE (US); Suresh B. Singh, Kendall Park, NJ (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 12/225,993

(22) PCT Filed: Apr. 5, 2007
(86) PCT No.: PCT/US07/08520

§ 371 Date: Jan. 28, 2009

Related U.S. Application Data
(60) Provisional application No. 60/789,823, filed on Apr. 5, 2006.
(60) Provisional application No. 60/789,703, filed on Apr. 5, 2006.

(51) Int. Cl. A61K 31/5377; C07D 211/32; A61K 31/445; A61K 31/4523; C07D 401/06; C07D 413/10; A61P 3/10

(52) U.S. Cl. 514/235.5; 546/226; 514/330; 514/210.18; 546/208; 544/131

(57) ABSTRACT

Described are compounds of the formula (I) which are orally active and bind to aspartic proteases to inhibit their activity. They are useful in the treatment or amelioration of diseases associated with aspartic protease activity. Also described are methods of use of the compounds described herein in ameliorating or treating aspartic protease related disorders in a subject in need thereof.

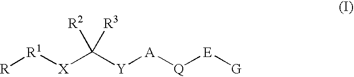

### RELATED APPLICATIONS

This application claims the benefit of U.S. Provisional Application No. 60/789,703, filed Apr. 5, 2006, and U.S. Provisional Application No. 60/789,823, filed Apr. 5, 2006, the entire teachings of which are incorporated herein by reference.

### BACKGROUND OF THE INVENTION

Aspartic proteases, including renin, β-secretase (BACE), Candida albicans secreted aspartyl proteases, HIV protease, HTLV protease and plasmepsins I and II, are implicated in a number of disease states. In hypertension elevated levels of angiotensin I, the product of renin catalyzed cleavage of angioteninogen are present. Elevated levels of βamyloid, the product of BACE activity on amyloid precursor protein, are widely believed to be responsible for the amyloid plaques present In the brains of Alzheimer's disease patients. Secreted aspartyl proteases play a role in the virulence of the pathogen Candida albicans. The viruses HIV and HTLV depend on their respective aspartic proteases for viral maturation. Plasmodium falciparum uses plasmepsins I and II to degrade hemoglobin.

In the renin-angiotensin-aldosterone system (RAAS) the biologically active peptide angiotensin II (Ang II) is generated by a two-step mechanism. The highly specific aspartic protease renin cleaves angiotensinogen to angiotensin I (Ang I), which is then further processed to Ang II by the less specific angiotensin-converting enzyme (ACE). Ang II is known to work on at least two receptor subtypes called AT₁ and AT₂. Whereas AT₁ seems to transmit most of the known functions of Ang II, the role of AT₂ is still unknown.

Modulation of the RAAS represents a major advance in the treatment of cardiovascular diseases (Zaman, M. A. et al Nature Reviews Drug Discovery 2002, 1, 621-636). ACE inhibitors and AT₁ blockers have been accepted as treatments of hypertension (Waeber B. et al., “The renin-angiotensin system: role in experimental and human hypertension”, in Berkenhager W. H., Reid J. L. (eds): Hypertension, Amsterdam, Elsevier Science Publishing Co, 1996, 489-519; Weber M. A., Am. J. Hypertens., 1992, 5, 247S). In addition, ACE inhibitors are used for renal protection (Rosenberg M. E. et al., Kidney International, 1994, 45, 403; Breyer J. A. et al, Kidney International, 1994, 45, S156), in the prevention of congestive heart failure (Vaughan D. E. et al., Cardiovasc. Res., 1994, 28, 159; Fouad-Tarazi F. et al., Am. J. Med., 1988, 84 (Suppl. 3A), 83) and myocardial infarction (Pfeffer M. A. et al., N Engl. J: Med. 1992, 327, 669).

Interest in the development of renin inhibitors stems from the specificity of renin (Kleinert H. D., Cardiovasc. Drugs, 1995, 9, 645). The only substrate known for renin is angiotensinogen, which can only be processed (under physiological conditions) by renin. In contrast, ACE can also cleave bradykinin besides Ang I and can be bypassed by chymase, a serine protease (Husain A., J. Hypertens., 1993, 11, 1155). In patients, inhibition of ACE thus leads to bradykinin accumulation causing cough (5-20%) and potentially life-threatening angioneurotic edema (0.1-0.2%) (Israili Z. H. et al., Annals of Internal Medicine, 1992, 117, 234). Chymase is not inhibited by ACE inhibitors. Therefore, the formation of Ang II is still possible in patients treated with ACE inhibitors. Blockade of the AT1 receptor (e.g., by losartan) on the other hand overexposes other AT-receptor subtypes to Ang II, whose concentration is dramatically increased by the blockade of AT1 receptors. In summary, renin inhibitors are not only expected to be superior to ACE inhibitors and AT₁ blockers with regard to safety, but more importantly also with regard to their efficacy in blocking the RAAS.

Only limited clinical experience (Azizi M. et al., J. Hypertens., 1994, 12, 419; Neutel J. M. et al., Am. Heart, 1991, 122, 1094) has been generated with renin inhibitors because their peptidomimetic character imparts insufficient oral activity (Kleinert H. D., Cardiovasc. Drugs, 1995, 9, 645). The clinical development of several compounds has been stopped because of this problem together with the high cost of goods. It appears as though only one compound has entered clinical trials (Rahuel J. et al., Chem. Biol., 2000, 7, 493; Mealy N. E., Drugs of the Future, 2001, 26, 1139). Thus, metabolically stable, orally bioavailable and sufficiently soluble renin inhibitors that can be prepared on a large scale are not available. Recently, the first non-peptide renin inhibitors were described which show high in vitro activity (Oefner C. et al., Chem. Biol., 1999, 6, 127; Patent Application WO 97/09311; Maerki H. P. et al., II Farmaco, 2001, 56, 21). The present invention relates to the unexpected identification of renin inhibitors of a non-peptidic nature and of low molecular weight. Orally active renin inhibitors which are active in indications beyond blood pressure regulation where the tissular renin-chymase system may be activated leading to pathophysiologically altered local functions such as renal, cardiac and vascular remodeling, atherosclerosis, and restenosis, are described.

All documents cited herein are incorporated by reference.

### SUMMARY OF THE INVENTION

Compounds have now been found which are orally active and bind to aspartic proteases to inhibit their activity. They are useful in the treatment or amelioration of diseases associated with aspartic protease activity.

In one embodiment the present invention is directed to compounds represented by Formula I:

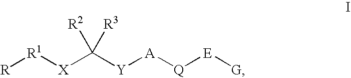

or an enantiomer, diastereomer or salt thereof.

R is:

a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cycloalkenyloxy, (C₃-C₇)cycloalkoxy(C₁-C₃)alkyl, (C₃-C₇)cycloalkyl(C₁-C₃)alkoxy, (C₁-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;

b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

c) a divalent radical selected from —CH₂)₃—, —(CH₂)₄—, —(CH₂)₅— or —(CH₂)₆—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo.

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl.

X and Y are each independently CH₂ or a single bond.

R² is:

a) —H; or

b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxycarbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkyl, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy.

The divalent sulfur atoms in R² are independently optionally oxidized to sulfoxide or sulfone and wherein the carbonyl groups are optionally independently changed to a thiocarbonyl groups;

R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylaminocarbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl, provided that

i) R² and R³ are not both hydrogen; and

ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:

1) 1 to 5 halogen atoms; and

2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, or halo(C₃-C₆)cycloalkoxy.

The divalent sulfur atoms in R³ are independently optionally oxidized to sulfoxide or sulfone and wherein the carbonyl groups in R³ are optionally independently changed to thiocarbonyl groups.

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively, wherein m is 1 to 3.

Q and Y are attached to carbon or nitrogen atoms in ring A in a 1, 2 or 1,3, or 1,4 relationship.

Q is a divalent radical selected from

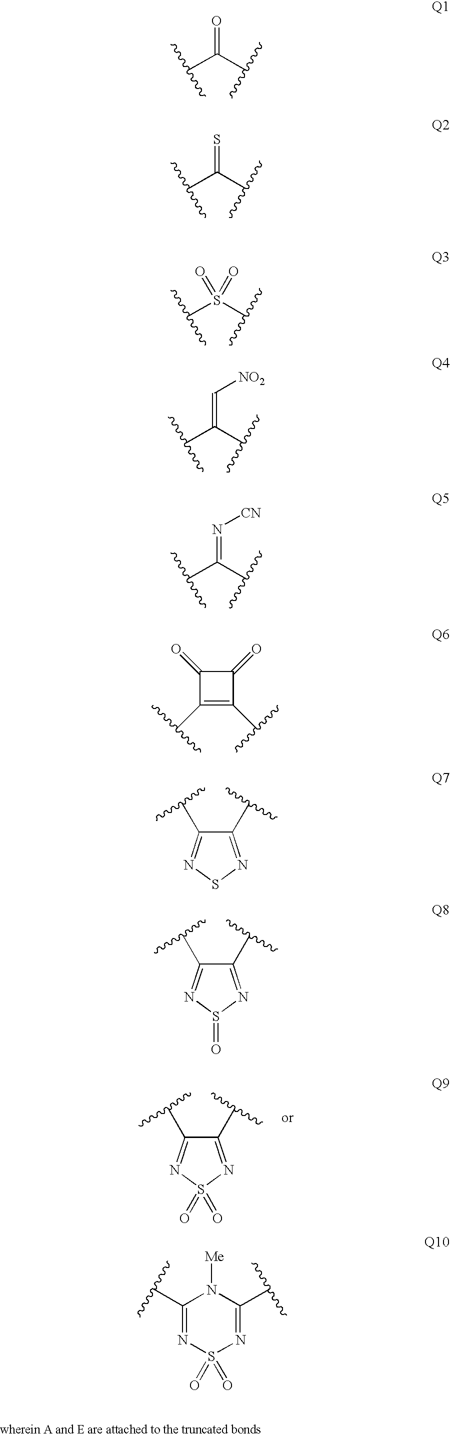

E is a saturated or unsaturated 3-, 4-, 5-, 6-, or 7-membered ring which is optionally bridged by (CH₂), via bonds to two members of said ring, wherein said ring is composed of carbon atoms, and 0-3 hetero atoms selected from 0, 1, 2. or 3 nitrogen atoms, 0 or 1 oxygen atoms, and 0 or 1 sulfur atoms, said ring being optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively, wherein n is 1 to 3.

G is hydroxy, hydroxy(C₁-C₆)alkyl, amino, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NBC(═NH)NHR⁴, wherein R⁴ is (C₁-C₃)alkyl.

In another embodiment the present invention is directed to pharmaceutical compositions comprising a compound described herein or enantiomers, diastereomers, or salts thereof and a pharmaceutically acceptable carrier or excipient.

In another embodiment the present invention is directed to a method of antagonizing aspartic protease inhibitors in a subject in need thereof comprising administering to the subject a therapeutically effective amount of a compound described herein or an enantiomer, diastereomer, or salt thereof.

In another embodiment the present invention is directed to method for treating or ameliorating an aspartic protease mediated disorder in a subject in need thereof comprising administering to said subject a therapeutically effective amount of a compound described herein or an enantiomer, diastereomer, or salt thereof.

In another embodiment the present invention is directed to a method for treating or ameliorating a renin mediated disorder in a subject in need thereof comprising administering to the subject an effective amount of a compound described herein or an enantiomer, diastereomer, or salt thereof.

In another embodiment the present invention is directed to a method for the treatment of hypertension in a subject in need thereof comprising administering to the subject a compound described herein in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

### DETAILED DESCRIPTION OF THE INVENTION

A description of embodiments of the compounds of Formula I of the invention follows. It is understood that the invention encompasses all combinations of the substituent variables (i.e., R, R¹, R², R³, etc.) defined herein.

In one embodiment of this invention, R is (1) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cyclo-alkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from: fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or

(2) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)₆-cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or

(3) R is a divalent radical selected from —(CH₂)₃—, —(CH₂)₄—, —(CH₂)₅— or —(CH₂)₆—, which is attached to R¹ to form a fused or spirofused ring system, and is optionally substituted with up to four substituents independently selected from: fluorine, hydroxy, (C₁-C₆)alkyl, halo(C₁-C₆)alkyl, (C₁-C₆)alkoxy and oxo.

In a particular embodiment of this invention, R is (1) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)-alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of: fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo; or

(2) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of: fluorine, chlorine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cyclo-alkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NCO, H₂NSO₂, (C₁-C₆)alkylaminocarbonyl, and (C₁-C₆)alkylaminosulfonyl; or

(3) R is a divalent radical selected from —(CH₂)₄— or —CH₂)₅—, which is attached to R¹ to form a fused or spirofused ring system, and is optionally substituted with up to four substituents independently selected from: fluorine, hydroxy, (C₁-C₆)alkyl, halo(C₁-C₆)alkyl, (C₁-C₆)alkoxy and oxo.

In another particular embodiment, R is (1) (C₁-C₈)alkyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkylethenyl, (C₃-C₇)cycloalkylethynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, and halo(C₁-C₃)alkyl, or (2) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or (3) a divalent radical selected from —(CH₂)₄— or —(CH₂)₅—, which is attached to R¹ to form a fused or spirofused ring system.

In a further particular embodiment of this invention, R is (1) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, and halo(C₁-C₃)alkyl; or (2) phenyl, monocyclic heteroaryl, phenoxy, monocyclic heteroaryloxy, phenyl(C₁-C₃)alkoxy, and monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio, and H₂NCO; or (3) —(CH₂)₄— or —(CH₂)₅—. In specific embodiments of this invention, R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, or piperidino, trimethylsilyl, —(CH₂)₄— or —CH₂)₅—.

R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl, and di(C₁-C₆)alkylaminocarbonyl.

In a particular embodiment of this invention, R¹ is a phenyl, monocyclic heteroaryl, bicyclic heteroaryl, benzo-1,3-dioxole, or (C₃-C₇)cycloalkyl ring optionally substituted with up to four substituents independently selected from the group consisting of: fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl.

In another particular embodiment of this invention, R¹ is a phenyl, monocyclic heteroaryl ring, bicyclic heteroaryl ring or benzo-1,3-dioxole, optionally substituted with up to four substituents independently selected from: halogen, cyano, (C₁-C₃)alkyl, (C₃-C₄)cycloalkyl, halo(C₁-C₃)alkyl, (C₁-C₃)alkoxy, halo(C₁-C₃)alkoxy, and H₂NCO. In a further embodiment of this invention, R¹ is a phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from fluorine, chlorine, cyano, (C₁-C₃)alkyl, halo(C₁-C₃)alkyl, (C₁-C₃)alkoxy, and carboxamide. In specific embodiments of this invention, R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzoxazole, benzo-1,3-dioxole.

R² is (1) hydrogen or (2) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxycarbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkyl, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, wherein (1) each optionally substituted by (a) 1 to 5 halogen atoms and (b) by 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone and wherein (3) a carbonyl group is optionally changed to a thiocarbonyl group.

In a particular embodiment of this invention, R² is (1) hydrogen or (2) (C₁-C₁₀)alkyl, (C₂-C₁₀)alkenyl, (C₂-C₁₀)alkynyl, (C₁-C₁₀)alkoxy, (C₁-C₁₀)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₈)alkylthio(C₁-C₅)alkylamino, (C₁-C₅)alkylthio(C₁-C₅)alkylthio, (C₁-C₅)alkylamino(C₁-C₅)alkoxy, (C₇-C₅)alkylamino(C₁-C₅)alkylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino-(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)alkylamino, (C₁-C₅)alkoxycarbonylamino(C₁-C₅)alkyl, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkyl, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₈)alkylthio, aminocarbonyl(C₁-C₈)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylaminocarboxy(C₁-C₅)alkoxy, (C₁-C₅)alkylaminocarboxy(C₁-C₈)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, wherein (1) each are optionally substituted by (a) 1 to 5 fluorine atoms and (b) by 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy and wherein (2) divalent sulfur atoms are optionally oxidized to sulfoxide or sulfone.

In another particular embodiment of this invention, R² is hydrogen, (C₁-C₈)alkyl, (C₄-C₈)cycloalkylalkyl, fluoro(C₁-C₈)alkyl, fluoro(C₄-C₉)-cycloalkylalkyl, (C₁-C₈)alkoxy, (C₄-C₈)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₈)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)cycloalkanecarbonylamino(C₁-C₅)alkyl, (C₃-C₄)cycloalkanecarbonylamino(C₁-C₅)alkoxy, aminosulfonylamino(C₁-C₈)alkyl, aminosulfonylamino(C₁-C₈)alkoxy, (C₁-C₅)alkanesulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, (C₁-C₅)alkylaminocarbonylamino(C₁-C₈)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₈)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino. In a further particular embodiment of this invention, R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₄)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy. In specific embodiments of this invention, R² is 4-methoxybutyl, 4-ethoxybutyl, 4-methoxypentyl, 3-methoxypropoxy, 3-(methoxycarbonylamino)propyl, 3-(acetylamino)propyl, 2-(acetylamino)ethoxy, or 2-(methoxycarbonylamino)ethoxy.

R³ is H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)-alkoxycarbonylamino, (C₁-C₆)alkylaminocarbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, or phenylamino or heteroarylamino in which each phenylamino and heteroarylamino group is optionally substituted with 1 to 5 groups independently selected from the group consisting of: fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl; provided that (i) R² and R³ are not both hydrogen and (ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)-acyloxy(C₁-C₆)alkylamino, aminosulfonylamino(C₁-C₂)alkoxy, aminosulfonylamino(C₁-C₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonyl-amino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, wherein (1) each optionally substituted by (a) 1 to 5 halogen atoms and (b) by 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy and wherein (2) divalent sulfur atoms are optionally oxidized to sulfoxide or sulfone and wherein (3) a carbonyl group is optionally changed to a thiocarbonyl group.

In another particular embodiment of this invention, R³ is H, halogen, (C₁-C₃)alkyl, (C₁-C₃)alkoxy, hydroxyl, hydroxy(C₁-C₃)alkyl, hydroxy(C₁-C₃)alkoxy, (C₁-C₄)alkanoylamino, (C₁-C₃)alkoxycarbonylamino, (C₁-C₃)alkylamino-carbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from: fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl; provided that (i) R² and R³ are not both hydrogen and (ii) when R³ is hydroxyl, halogen, or optionally substituted phenylamino or heteroarylamino, R² is not (C₁-C₁₀)alkoxy, (C₁-C₁₀)alkylthio, (C₁-C₁₀)alkylamino, (C₁-C₈)alkylthio(C₁-C₈)alkyl, (C₁-C₅)alkoxy(C₁-C₅)alkoxy, (C₁-C₅)alkoxy(C₁-C₅)alkylthio, (C₁-C₅)alkoxy(C₁-C₈)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₈)alkylthio, (C₁-C₅)alkylamino(C₁-C₅)alkylamino, aminocarbonylamino(C₁-C₁₀)alkoxy, aminocarbonylamino(C₁-C₁₀)alkylthio, aminocarbonyl-amino(C₁-C₁₀)alkylamino, (C₁-C₅)alkanoylamino(C₁-C₅)alkoxy, (C₁-C₅)alkanoylamino(C₁-C₅)alkylthio, (C₁-C₅)alkanoylamino(C₁-C₈)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)-alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, wherein (1) each are optionally substituted by (a) 1 to 5 fluorine atoms and (b) by 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy and wherein (2) divalent sulfur atoms are optionally oxidized to sulfoxide or sulfone.

In a further particular embodiment of this invention, R³ is H, halogen, OH, (C₁-C₄)alkanoylamino, or (C₁-C₃)alkoxy; provided that (i) R² and R³ are not both hydrogen and (ii) when R³ is OH or halogen, R² is not (C₁-C₈)alkoxy, (C₄-C₉)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)-alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkanecarbonylamino(C₁-C₅)alkoxy, aminosulfonylamino(C₁-C₈)alkoxy, (C₁-C₈)alkanesulfonylamino(C₁-C₅)alkoxy, formylamino(C₁-C₅)alkoxy, (C₁-C₈)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₈)alkoxy, aminocarboxy(C₁-C₅)alkoxy, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁₋₈)alkanoylamino. In specific embodiments of this invention, R³ is hydrogen or hydroxyl provided that when R³ is hydroxyl, R² is not 3-methoxypropoxy, 2-(acetylamino)ethoxy, or 2-(methoxycarbonylamino)ethoxy.

A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring atoms being substituted with the appropriate number of hydrogen atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively; and, wherein m is 1 to 3. In a particular embodiment of this invention, Ring A is piperidine, morpholine or benzene.

Q and Y are attached to carbon or nitrogen atoms in ring A in a 1, 2 or 1,3, or 1,4 relationship; X and Y are each independently CH₂ or a single bond. In the specific embodiments of this invention, X and Y are each a single bond.

In one particular embodiment of this invention, Q is a divalent radical selected from

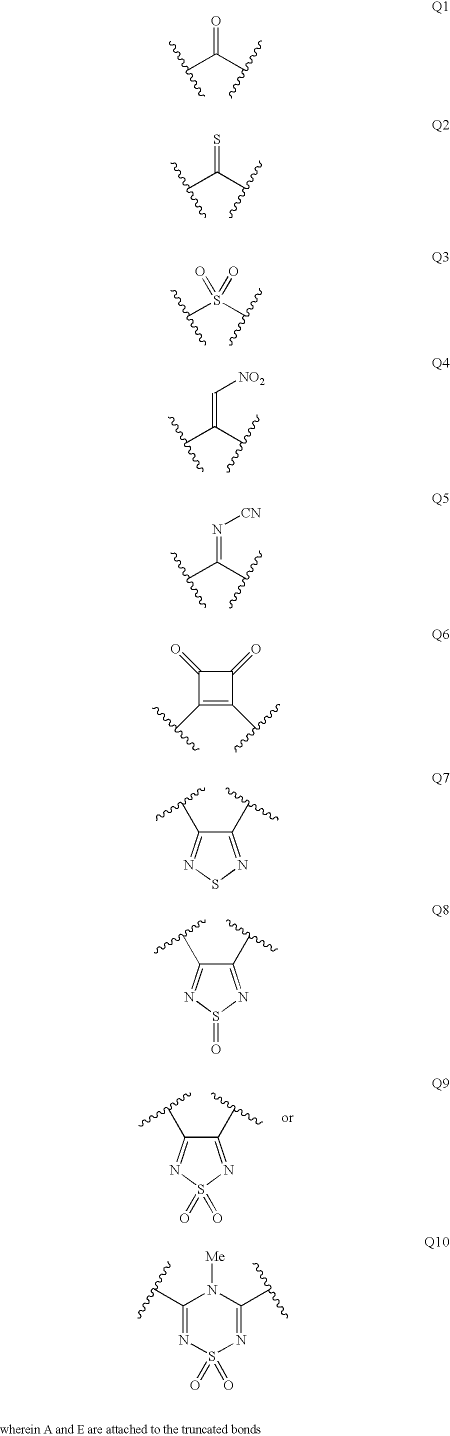

In another particular embodiment of this invention, Q is a divalent radical selected from Q1, Q2, Q3, Q4, Q5, Q6, and Q7. In another embodiment of this invention, Q is Q1, Q2, Q4, or Q6. In specific embodiments of this invention, Q is Q1, Q4, or Q6.

E is a saturated or unsaturated 3-, 4-, 5-, 6-, or 7-membered ring which is optionally bridged by (CH₂)ₙ via bonds to two members of said ring, wherein said ring is composed of carbon atoms, and 0-3 hetero atoms selected from 0, 1, 2, or 3 nitrogen atoms, 0 or 1 oxygen atoms, and 0 or 1 sulfur atoms, said ring atoms being substituted with the appropriate number of hydrogen atoms, said ring being optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively; and, wherein n is 1 to 3. In a particular embodiment of this invention, E is a saturated 3-, 4-, 5-, 6-, or 7-membered ring or an unsaturated 5- or 6-membered ring composed of carbon atoms and 0-3 hetero atoms selected from 0, 1, 2, or 3 nitrogen atoms, 0 or 1 oxygen atoms, and 0 or 1 sulfur atoms, said ring atoms being substituted with the appropriate number of hydrogen atoms, said ring being optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively.

In another particular embodiment of this invention, E is a saturated 3-, 4-, 5-, or 6-membered ring or an unsaturated 5- or 6-membered ring, wherein said ring is composed of carbon atoms, and 0-3 hetero atoms selected from 0, 1, 2, or 3 nitrogen atoms, 0 or 1 oxygen atoms, and 0 or 1 sulfur atoms, said ring atoms being substituted with the appropriate number of hydrogen atoms, said ring being optionally substituted with up to four groups independently selected from fluorine, hydroxy, (C₁-C₃)alkyl, hydroxy(C₁-C₃)alkyl, and oxo groups such that when there is substitution with one oxo group on a carbon atom it forms a carbonyl group and when there is substitution of one or two oxo groups on sulfur it forms sulfoxide or sulfone groups, respectively. In another embodiment of this invention, E is a saturated 3-, 4-, 5-, or 6-membered ring or an unsaturated 5- or 6-membered ring composed of carbon atoms and 0 or 1 nitrogen atoms, said ring being optionally substituted with up to one hydroxy or hydroxy(C₁-C₃)alkyl group and with up to two (C₁-C₃) alkyl groups. In specific embodiments of this invention, E is azetidine, pyrrolidine, hydroxypyrrolidine, (hydroxymethyl)pyrrolidine, methylpyrrolidine, piperidine, hydroxypiperidine, cyclopropane, methylcyclopropane, cyclopentane, hydroxycyclopentane, cyclohexane, hydroxycyclohexane, or pyridine.

G is hydroxy, hydroxy(C₁-C₆)alkyl, amino, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴; where R⁴ is (C₁-C₃)alkyl. In a particular embodiment of this invention, G is hydroxy, hydroxy(C₁-C₃)alkyl, amino, (C₁-C₃)alkylamino, amino(C₁-C₃)alkyl, or (C₁-C₃)alkylamino(C₁-C₃)alkyl. In another particular embodiment of this invention, G is amino, (C₁-C₃)alkylamino, amino(C₁-C₃)alkyl, or (C₁-C₃)alkylamino(C₁-C₃)alkyl. In another embodiment of this invention, G is amino, amino(C₁-C₃)alkyl, (C₁-C₃)alkylamino, or (C₁-C₃)alkylamino(C₁-C₃)alkyl. In specific embodiments of this invention, G is amino, aminomethyl, methylamino or methylaminomethyl.

An embodiment of the invention is a compound of Formula Ia:

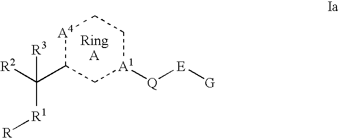

wherein R, R¹, R², R³, Q, E and G are as defined above for Formula I, and Ring A is a benzene ring (A¹ and A⁴ are CH and the bonds in ring A are aromatic bonds); or Ring A is a piperidine ring (A¹ is N, A⁴ is CH₂ and the bonds in ring A are single bonds); or Ring A is a morpholine ring (A¹ is N, A⁴ is O and the bonds in ring A are single bonds), or an enantiomer, diastereomer or salt thereof.

Another embodiment of the invention is a compound of Formula Ia wherein:

R is (1) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)-alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of:
fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo;
   or (2) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of:
fluorine, chlorine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cyclo-alkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂₇CO, H₂NSO₂, (C₁-C₆)alkylaminocarbonyl, and (C₁-C₆)alkylaminosulfonyl;
   or (3) R is a divalent radical selected from —(CH₂)₄— or —CH₂)₅—, which is attached to R¹ to form a fused or spirofused ring system, and is optionally substituted with up to four substituents independently selected from:

fluorine, hydroxy, (C₁-C₆)alkyl, halo(C₁-C₆)alkyl, (C₁-C₆)alkoxy and oxo;

R¹ is a phenyl, monocyclic heteroaryl, bicyclic heteroaryl, benzo-1,3-dioxole, or (C₃-C₇)cycloalkyl ring optionally substituted with up to four substituents independently selected from the group consisting of:
fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl;
   R² is (1) hydrogen or (2) (C₁-C₁₀)alkyl, (C₂-C₁₀)alkenyl, (C₂-C₁₀)alkynyl, (C₁-C₁₀)alkoxy, (C₁-C₁₀)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino-(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)alkylamino, (C₁-C₅)alkoxycarbonylamino(C₁-C₅)alkyl, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkyl, (C₁-C₅)alkylaminocarbonylamino(C₁-C₈)alkoxy, (C₁-C₅)alkylaminocarbonylamino(C₁-C₈)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylaminocarboxy(C₁-C₅)alkoxy, (C₁-C₅)alkylaminocarboxy(C₁-C₅)alkylthio, (C₁-C₈)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, wherein (1) each are optionally substituted by (a) 1 to 5 fluorine atoms and (b) by 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy and wherein (2) divalent sulfur atoms are optionally oxidized to sulfoxide or sulfone;
   R³ is H, halogen, (C₁-C₃)alkyl, (C₁-C₃)alkoxy, hydroxyl, hydroxy(C₁-C₃)alkyl, hydroxy(C₁-C₃)alkoxy, (C₁-C₄)alkanoylamino, (C₁-C₃)alkoxycarbonylamino, (C₁-C₃)alkylaminocarbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from:
fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;
   provided that (i) R² and R³ are not both hydrogen and (ii) when R³ is hydroxyl, halogen, or optionally substituted phenylamino or heteroarylamino, R² is not (C₁-C₁₀)alkoxy, (C₁-C₁₀)alkylthio, (C₁-C₁₀)alkylamino, (C₁-C₅)alkylthio(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, aminocarbonylamino(C₁-C₁₀)alkoxy, aminocarbonylamino(C₁-C₁₀)alkylthio, aminocarbonylamino(C₁-C₁₀)alkylamino, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₈)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, wherein (1) each are optionally substituted by (a) 1 to 5 fluorine atoms and (b) by 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy and wherein (2) divalent sulfur atoms are optionally oxidized to sulfoxide or sulfone;
   Ring A is a benzene ring (A¹ and A⁴ are CH and the bonds in ring A are aromatic bonds); or
   Ring A is piperidine, A¹ is N, A⁴ is CH₂ and the bonds in ring A are single bonds; or
   Ring A is morpholine, A¹ is N, A⁴ is 0 and the bonds in ring A are single bonds;
   Q is a divalent radical selected from Q1, Q2, Q3, Q4, Q5, Q6, and Q7;
   E is a saturated 3-, 4-, 5-, 6-, or 7-membered ring or an unsaturated 5- or 6-membered ring composed of carbon atoms and 0-3 hetero atoms selected from 0, 1, 2, or 3 nitrogen atoms, 0 or 1 oxygen atoms, and 0 or 1 sulfur atoms, said ring atoms being substituted with the appropriate number of hydrogen atoms, said ring being optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively;
   G is hydroxy, hydroxy(C₁-C₃)alkyl, amino, (C₁-C₃)alkylamino, amino(C₁-C₃)alkyl, or (C₁-C₃)alkylamino(C₁-C₃)alkyl;
   or an enantiomer, diastereomer or salt thereof.

Specific and particular values for each variable in Formula Ia are as described above for Formula I.

An embodiment of the invention is a compound of Formula I or Ia wherein:

R is (1) (C₁-C₈)alkyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkylethenyl, (C₃-C₇)cycloalkylethynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, and halo(C₁-C₃)alkyl, or (2) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or (3) a divalent radical selected from —(CH₂)₄— or —(CH₂)₅—, which is attached to R¹ to form a fused or spirofused ring system,
R¹ is a phenyl, monocyclic heteroaryl ring, bicyclic heteroaryl ring or benzo-1,3-dioxole, optionally substituted with up to four substituents independently selected from:
halogen, cyano, (C₁-C₃)alkyl, (C₃-C₄)cycloalkyl, halo(C₁-C₃)alkyl, (C₁-C₃)alkoxy, halo(C₁-C₃)alkoxy, and H₂NCO;
   R² is hydrogen, (C₁-C₈)alkyl, (C₄-C₉)cycloalkylalkyl, fluoro(C₁-C₈)alkyl, fluoro(C₄-C₉)cycloalkylalkyl, (C₁-C₈)alkoxy, (C₄-C₉)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₈)alkoxy(C₁-C₃)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)cycloalkanecarbonylamino(C₁-C₅)alkyl, (C₃-C₄)cycloalkanecarbonylamino(C₁-C₅)alkoxy, aminosulfonylamino(C₁-C₈)alkyl, aminosulfonylamino(C₁-C₈)alkoxy, (C₁-C₅)alkanesulfonylamino(C₁-C₅)alkyl, (C₁-C₅)alkanesulfonylamino(C₁-C₅)alkoxy, formylamino(C₁-C₅)alkyl, formylamino(C₁-C₈)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, (C₁-C₅)alkylaminocarbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;
   R³ is H, halogen, OH, (C₁-C₄)alkanoylamino, or (C₁-C₃)alkoxy;
   provided that (i) R² and R³ are not both hydrogen and (ii) when R³ is OH or halogen, R² is not (C₁-C₈)alkoxy, (C₄-C₉)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkanecarbonylamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₈)alkoxy, aminocarboxy(C₁-C₈)alkoxy, (C₁-C₅)alkylaminocarboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;
   Ring A is piperidine, morpholine or benzene;

### Q is Q1, Q2, Q4, or Q6;

E is a saturated 3-, 4-, 5-, or 6-membered ring or an unsaturated 5- or 6-membered ring, wherein said ring is composed of carbon atoms, and 0-3 hetero atoms selected from 0, 1, 2, or 3 nitrogen atoms, 0 or 1 oxygen atoms, and 0 or 1 sulfur atoms, said ring atoms being substituted with the appropriate number of hydrogen atoms, said ring being optionally substituted with up to four groups independently selected from fluorine, hydroxy, (C₁-C₃)alkyl, hydroxy(C₁-C₃)alkyl, and oxo groups such that when there is substitution with one oxo group on a carbon atom it forms a carbonyl group and when there is substitution of one or two oxo groups on sulfur it forms sulfoxide or sulfone groups, respectively
G is amino, (C₁-C₃)alkylamino, amino(C₁-C₃)alkyl, or (C₁-C₃)alkylamino(C₁-C₃)alkyl; or an enantiomer, diastereomer or salt thereof.

An embodiment of the invention is a compound of Formula I or Ia wherein: R is (1) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, and halo(C₁-C₃)alkyl; or (2) phenyl, monocyclic heteroaryl, phenoxy, monocyclic heteroaryloxy, phenyl(C₁-C₃)alkoxy, and monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio, and H₂NCO; or (3) —CH₂)₄— or —(CH₂)₅—;

R¹ is a phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from fluorine, chlorine, cyano, (C₁-C₃)alkyl, halo(C₁-C₃)alkyl, (C₁-C₃)alkoxy, and carboxamide;
R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₄)cycloalkyl(C₁-C₅)alkoxy, (C₁-C₃)alkoxycarbonylamino(C₁-C₃)alkyl, (C₁-C₃)alkoxycarbonylamino(C₁-C₅)alkoxy, (C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy;
R³ is hydrogen, fluoro, hydroxyl, or (C₁-C₄)alkanoylamino, provided that when R³ is hydroxyl or fluoro, R² is not (C₁-C₃)alkoxy(C₁-C₅)alkoxy, (C₃-C₄)cycloalkyl(C₁-C₅)alkoxy, (C₁-C₃)alkoxycarbonylamino(C₁-C₅)alkoxy, (C₁-C₃)alkanoylamino(C₁-C₅)alkoxy or (C₁-C₃)alkylaminocarbonyl(C₁-C₅)alkoxy;
Ring A is piperidine, morpholine, or benzene

### Q is Q1, Q2, Q4. or Q6;

E is a saturated 3-, 4-, 5-, or 6-membered ring or an unsaturated 5- or 6-membered ring composed of carbon atoms and 0 or 1 nitrogen atoms, said ring being optionally substituted with up to one hydroxy or hydroxy(C₁-C₃)alkyl group and with up to two (C₁-C₃) alkyl groups;
G is amino, amino(C₁-C₃)alkyl, (C₁-C₃)alkylamino, or (C₁-C₃)alkylamino(C₁-C₃)alkyl;
or an enantiomer, diastereomer or salt thereof.

An embodiment of the invention is a compound of Formula I wherein:

R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, or piperidino, trimethylsilyl, —(CH₂)₄— or —(CH₂)₅—;

R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzoxazole, benzo-1,3-dioxole
R² is 4-methoxybutyl, 4-ethoxybutyl, 4-methoxypentyl, 3-methoxypropoxy, 3-(methoxycarbonylamino)propyl, 3-(acetylamino)propyl, 2-(acetylamino)ethoxy, or 2-(methoxycarbonylamino)ethoxy;
R³ is hydrogen or hydroxyl provided that when R³ is hydroxyl, R² is not 3-methoxypropoxy, 2-(acetylamino)ethoxy, or 2-(methoxycarbonylamino)ethoxy;
Ring A is piperidine, morpholine, or benzene;

### Q is Q1, Q4, or Q6;

E is azetidine, pyrrolidine, hydroxypyrrolidine, (hydroxymethyl)pyrrolidine, methylpyrrolidine, piperidine, hydroxypiperidine, cyclopropane, methylcyclopropane, cyclopentane, hydroxycyclopentane, cyclohexane, hydroxycyclohexane, or pyridine;
G is amino, aminomethyl, methylamino or methylaminomethyl;
or an enantiomer, diastereomer or salt thereof.

An embodiment of the invention is a compound of Formula Ia with the stereochemical configuration shown in Formula Ib:

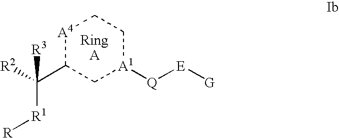

wherein R, R¹, R², R³, Ring A, A¹, A⁴, Q, E and G are as defined above for Formula Ia, or an enantiomer, diastereomer or salt thereof. Specific and particular values for each variable in Formula Ib are as described for Formula I.

An embodiment of invention is a compound of Formula Ic:

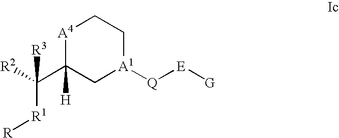

wherein R, R¹, R², R³, A¹, A⁴, Q, E and G are as defined above for Formula Ia, or an enantiomer, diastereomer or salt thereof. Specific and particular values for each variable in Formula Ic are as described for Formula I.

An embodiment of invention is a compound of Formula Ia wherein E is a cyclopentane ring with the stereochemical configuration shown in Formula Id

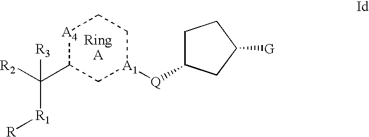

wherein R, R¹, R², R³, Ring A, A¹, A⁴, Q, and G are as defined above for Formula Ia or an enantiomer, diastereomer or salt thereof. Specific and particular values for each variable in Formula Id are as described for Formula I.

An embodiment of invention is a compound of Formula Ia wherein E is a pyrrolidine ring with the stereochemical configuration shown in Formula Ie

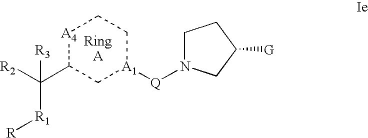

wherein R, R¹, R², R³, Ring A, A¹, A⁴, Q, and G are as defined above for Formula Ia or an enantiomer, diastereomer or salt thereof. Specific and particular values for each variable in Formula Ie are as described for Formula I.

The following are compounds of the invention:

| Cpd. No. | Name |
|---|---|
| I-1 | (3-aminocyclopentyl)(3-(1-(2-phenylphenyl)-1-hydroxy-5-methoxypentyl)piperidin-1- |
| | yl)methanone |
| I-2 | (3-aminopyrrolidin-1-yl)(3-(1-(2-phenylphenyl)-1-hydroxy-5-methoxypentyl)piperidin- |
| | 1-yl)methanone |
| I-3 | (3-amino-4-hydroxycyclopentyl)(3-(1-(2-(cyclopropylmethoxy)phenyl)-1-hydroxy-5- |
| | methoxypentyl)piperidin-1-yl)methanone |
| I-3 | (3-amino-4-hydroxycyclopentyl)(3-(1-(2-(cyclopropylmethoxy)phenyl)-1-hydroxy-5- |
| | methoxypentyl)piperidin-1-yl)methanone |
| I-4 | (3-aminocyclopentyl)(3-(1-hydroxy-5-methoxy-1-(2-phenoxyphenyl)pentyl)piperidin-1- |
| | yl)methanone |
| I-5 | (3-amino-4-hydroxycyclopentyl)(3-(1-(2-phenylphenyl)-1-hydroxy-5- |
| | methoxypentyl)piperidin-1-yl)methanone |
| I-6 | (3-aminopyrrolidin-1-yl)(3-(1-hydroxy-5-methoxy-1-(2- |
| | phenoxyphenyl)pentyl)piperidin-1-yl)methanone |
| I-7 | (3-(1-(2-(o-tolyloxy)phenyl)-1-hydroxy-5-methoxypentyl)piperidin-1-yl)(3- |
| | aminoazetidin-1-yl)methanone |
| I-8 | (3-aminopyrrolidin-1-yl)(3-(1-(2-phenyl-3-fluorophenyl)-1-hydroxy-5- |
| | methoxypentyl)piperidin-1-yl)methanone |
| I-9 | (3-(1-(2-(o-tolyloxy)phenyl)-1-hydroxy-5-methoxypentyl)phenyl)(3-aminopyrrolidin-1- |
| | yl)methanone |
| I-10 | (3-(1-(2-(2-cyclopropylethoxy)phenyl)-1-hydroxy-5-methoxypentyl)piperidin-1-yl)(3- |
| | amino-4-hydroxycyclopentyl)methanone |
| I-11 | (3-aminocyclopentyl)(3-(1-hydroxy-5-methoxy-1-(2′-methylbiphenyl-2- |
| | yl)pentyl)piperidin-1-yl)methanone |
| I-12 | (3-amino-4-hydroxycyclopentyl)(3-(1-(2-(cyclobutylmethoxy)phenyl)-1-hydroxy-5- |
| | methoxypentyl)piperidin-1-yl)methanone |
| I-13 | (3-amino-4-hydroxycyclopentyl)(3-(1-(2-(cyclopentyloxy)phenyl)-1-hydroxy-5- |
| | methoxypentyl)piperidin-1-yl)methanone |
| I-14 | (2-aminopyridin-4-yl)(3-(1-hydroxy-5-methoxy-1-(2-phenoxyphenyl)pentyl)piperidin- |
| | 1-yl)methanone |
| I-15 | (6-aminopyridin-3-yl)(3-(1-hydroxy-5-methoxy-1-(2-phenoxyphenyl)pentyl)piperidin- |
| | 1-yl)methanone |
| I-16 | (3-amino-4-hydroxycyclopentyl)(3-(1-hydroxy-5-methoxy-1-(2- |
| | (neopentyloxy)phenyl)pentyl)piperidin-1-yl)methanone |
| I-17 | (3-aminocyclohexyl)(3-(1-hydroxy-5-methoxy-1-(2-phenoxyphenyl)pentyl)piperidin-1- |
| | yl)methanone |
| I-18 | (3-(1-(2-(o-tolyloxy)phenyl)-1-hydroxy-5-methoxypentyl)piperidin-1-yl)(3- |
| | aminocyclopentyl)methanone |
| I-19 | (3-aminocyclopentyl)(3-(1-hydroxy-5-methoxy-1-(3′-methylbiphenyl-2- |
| | yl)pentyl)piperidin-1-yl)methanone |
| I-20 | (3-amino-4-hydroxycyclopentyl)(3-(1-(2-(3-methylphenyl)phenyl)-1-hydroxy-5- |
| | methoxypentyl)piperidin-1-yl)methanone |
| I-21 | (3-aminopiperidin-1-yl)(3-(1-hydroxy-5-methoxy-1-(2-phenoxyphenyl)pentyl)piperidin- |
| | 1-yl)methanone |
| I-22 | (3-(1-(2-(o-tolyloxy)phenyl)-1-hydroxy-5-methoxypentyl)piperidin-1-yl)(3- |
| | aminopyrrolidin-1-yl)methanone |
| I-23 | (3-(1-(2-(m-tolyloxy)phenyl)-1-hydroxy-5-methoxypentyl)piperidin-1-yl)(3- |
| | aminopyrrolidin-1-yl)methanone |
| I-24 | (3-(1-(2-(o-tolyloxy)phenyl)-1-hydroxy-5-methoxypentyl)piperidin-1-yl)(2- |
| | (aminomethyl)azetidin-1-yl)methanone |
| I-25 | (3-amino-4-hydroxycyclopentyl)(3-(1-hydroxy-5-methoxy-1-(2- |
| | phenoxyphenyl)pentyl)piperidin-1-yl)methanone |
| I-26 | (3-aminocyclopentyl)(3-(1-hydroxy-5-methoxy-1-(4′-methylbiphenyl-2- |
| | yl)pentyl)piperidin-1-yl)methanone |
| I-27 | (3-((3-methoxypropoxy)(2-(o-tolyloxy)phenyl)methyl)piperidin-1-yl)(3-amino-4- |
| | hydroxycyclopentyl)methanone |
| I-28 | (3-(1-(2-(3-fluorophenoxy)phenyl)-1-hydroxy-5-methoxypentyl)piperidin-1-yl)(3- |
| | aminocyclopentyl)methanone |
| I-29 | (3-amino-4-hydroxycyclopentyl)(3-(1-(2-phenyl-3-fluorophenyl)-1-hydroxy-5- |
| | methoxypentyl)piperidin-1-yl)methanone |
| I-30 | (3-aminopyrrolidin-1-yl)(3-(1-(3-fluoro-2-phenoxyphenyl)-1-hydroxy-5- |
| | methoxypentyl)piperidin-1-yl)methanone |
| I-31 | (3-(1-(2-(3-fluorophenoxy)phenyl)-1-hydroxy-5-methoxypentyl)piperidin-1-yl)(3- |
| | aminopyrrolidin-1-yl)methanone |
| I-32 | (3-(1-(2-(4-fluorophenoxy)phenyl)-1-hydroxy-5-methoxypentyl)piperidin-1-yl)(3- |
| | aminopyrrolidin-1-yl)methanone |
| I-33 | (3-amino-4-hydroxycyclopentyl)(3-(1-(2-(cyclopentylmethoxy)phenyl)-1-hydroxy-5- |
| | methoxypentyl)piperidin-1-yl)methanone |
| I-34 | (3-(1-(2-(o-tolyloxy)phenyl)-1-hydroxy-5-methoxypentyl)piperidin-1-yl)(6- |
| | aminopyridin-3-yl)methanone |
| I-35 | (3-(1-(2-(o-tolyloxy)phenyl)-1-hydroxy-5-methoxypentyl)piperidin-1-yl)(2- |
| | aminopyridin-4-yl)methanone |
| I-36 | (3-(1-(2-(o-tolyloxy)phenyl)-1-hydroxy-5-methoxypentyl)phenyl)(3-amino-4- |
| | hydroxypyrrolidin-1-yl)methanone |
| I-37 | (3-(1-(2-(o-tolyloxy)phenyl)-1-hydroxy-5-methoxypentyl)piperidin-1-yl)(3- |
| | aminocyclohexyl)methanone |
| I-38 | (3-(1-(2-(2-ethylphenoxy)phenyl)-1-hydroxy-5-methoxypentyl)piperidin-1-yl)(3- |
| | aminopyrrolidin-1-yl)methanone |
| I-39 | (3-(1-(2-(o-tolyloxy)phenyl)-5-ethoxy-1-hydroxypentyl)piperidin-1-yl)(3- |
| | aminopyrrolidin-1-yl)methanone |
| I-40 | (3-(1-(2-(o-tolyloxy)phenyl)-1-hydroxy-5-methoxypentyl)piperidin-1-yl)(3- |
| | (methylamino)pyrrolidin-1-yl)methanone |
| I-41 | (3-(1-(2-(o-tolyloxy)phenyl)-1-hydroxy-5-methoxypentyl)piperidin-1-yl)(3-amino-4- |
| | hydroxycyclopentyl)methanone |
| I-42 | (3-aminopyrrolidin-1-yl)(3-(1-hydroxy-5-methoxy-1-(2′-methylbiphenyl-2- |
| | yl)pentyl)piperidin-1-yl)methanone |
| I-43 | (3-(1-(2-(m-tolyloxy)phenyl)-1-hydroxy-5-methoxypentyl)piperidin-1-yl)(3-amino-4- |
| | hydroxycyclopentyl)methanone |
| I-44 | (3-amino-4-hydroxycyclopentyl)(3-(1-(2-(benzyloxy)phenyl)-1-hydroxy-5- |
| | methoxypentyl)piperidin-1-yl)methanone |
| I-45 | (3-(1-(2-(o-tolyloxy)phenyl)-1-hydroxy-5-methoxypentyl)piperidin-1-yl)(3-amino-4- |
| | hydroxypyrrolidin-1-yl)methanone |
| I-46 | (3-(1-(2-(3-fluorophenoxy)phenyl)-1-hydroxy-5-methoxypentyl)piperidin-1-yl)(3- |
| | aminocyclohexyl)methanone |
| I-47 | (3-amino-4-hydroxycyclopentyl)(3-(1-(3-fluoro-2-phenoxyphenyl)-1-hydroxy-5- |
| | methoxypentyl)piperidin-1-yl)methanone |
| I-48 | (3-(1-(3-(4-fluorophenoxy)phenyl)-1-hydroxy-5-methoxypentyl)piperidin-1-yl)(3- |
| | amino-4-hydroxycyclopentyl)methanone |
| I-49 | (3-(1-(2-(3-fluorophenoxy)phenyl)-1-hydroxy-5-methoxypentyl)piperidin-1-yl)(3- |
| | amino-4-hydroxycyclopentyl)methanone |
| I-50 | (3-(1-(2-(4-fluorophenoxy)phenyl)-1-hydroxy-5-methoxypentyl)piperidin-1-yl)(3- |
| | amino-4-hydroxycyclopentyl)methanone |
| I-51 | (3-(1-(2-(2-fluorophenoxy)phenyl)-1-hydroxy-5-methoxypentyl)piperidin-1-yl)(3- |
| | amino-4-hydroxycyclopentyl)methanone |
| I-52 | (3-amino-4-hydroxycyclopentyl)(3-(1-(2-(2-chlorophenyl)phenyl)-1-hydroxy-5- |
| | methoxypentyl)piperidin-1-yl)methanone |
| I-53 | (3-amino-4-hydroxycyclopentyl)(3-(1-(3-chloro-2-phenylphenyl)-1-hydroxy-5- |
| | methoxypentyl)piperidin-1-yl)methanone |
| I-54 | (3-amino-4-hydroxypyrrolidin-1-yl)(3-(1-(3-fluoro-2-phenoxyphenyl)-1-hydroxy-5- |
| | methoxypentyl)piperidin-1-yl)methanone |
| I-55 | (3-amino-4-hydroxycyclopentyl)(3-(1-(2-(3,4-difluorophenyl)phenyl)-1-hydroxy-5- |
| | methoxypentyl)piperidin-1-yl)methanone |
| I-56 | (3-amino-4-hydroxycyclopentyl)(3-(1-(2-(cyclohexylmethoxy)phenyl)-1-hydroxy-5- |
| | methoxypentyl)piperidin-1-yl)methanone |
| I-57 | 1-(1-(1-(3-aminopyrrolidin-1-yl)-2-nitrovinyl)piperidin-3-yl)-5-methoxy-1-(2- |
| | phenoxyphenyl)pentan-1-ol |
| I-58 | (3-(1-(2-(o-tolyloxy)phenyl)-1-hydroxy-5-methoxypentyl)piperidin-1-yl)(3-hydroxy-4- |
| | (methylamino)cyclopentyl)methanone |
| I-59 | (3-(1-(2-(2-ethylphenoxy)phenyl)-1-hydroxy-5-methoxypentyl)piperidin-1-yl)(3-amino- |
| | 4-hydroxycyclopentyl)methanone |
| I-60 | (3-(1-(2-(o-tolyloxy)phenyl)-1-hydroxy-6-methoxyhexyl)piperidin-1-yl)(3-amino-4- |
| | hydroxycyclopentyl)methanone |
| I-61 | (3-(1-(2-(o-tolyloxy)phenyl)-5-ethoxy-1-hydroxypentyl)piperidin-1-yl)(3-amino-4- |
| | hydroxycyclopentyl)methanone |
| I-62 | (3-(1-(2-(o-tolyloxy)phenyl)-1-hydroxy-5-methoxypentyl)piperidin-1-yl)(3-amino-4- |
| | hydroxycyclohexyl)methanone |
| I-63 | (3-(1-(2-(o-tolyloxy)-3-methylphenyl)-1-hydroxy-5-methoxypentyl)piperidin-1-yl)(3- |
| | amino-4-hydroxycyclopentyl)methanone |
| I-64 | (3-(1-(2-(o-tolyloxy)-3-fluorophenyl)-1-hydroxy-5-methoxypentyl)piperidin-1-yl)(3- |
| | amino-4-hydroxycyclopentyl)methanone |
| I-65 | (3-(1-(2-(4-fluorobenzyloxy)phenyl)-1-hydroxy-5-methoxypentyl)piperidin-1-yl)(3- |
| | amino-4-hydroxycyclopentyl)methanone |
| I-66 | (3-(1-(2-(o-tolyloxy)-3-fluorophenyl)-1-hydroxy-5-methoxypentyl)piperidin-1-yl)(3- |
| | amino-4-hydroxycyclopentyl)methanone |
| I-67 | (3-(1-(2-(5-fluoro-2-methylphenoxy)phenyl)-1-hydroxy-5-methoxypentyl)piperidin-1- |
| | yl)(3-amino-4-hydroxycyclopentyl)methanone |
| I-68 | (3-(1-(2-(4-fluoro-2-methylphenoxy)phenyl)-1-hydroxy-5-methoxypentyl)piperidin-1- |
| | yl)(3-amino-4-hydroxycyclopentyl)methanone |
| I-69 | (3-(1-(2-(p-tolyloxy)-3-fluorophenyl)-1-hydroxy-5-methoxypentyl)piperidin-1-yl)(3- |
| | amino-4-hydroxycyclopentyl)methanone |
| I-70 | (3-(1-(2-(o-tolyloxy)-3-fluorophenyl)-1-hydroxy-5-methoxypentyl)piperidin-1-yl)(3- |
| | amino-4-hydroxypyrrolidin-1-yl)methanone |
| I-71 | (3-(1-(2-(2-chlorophenoxy)phenyl)-1-hydroxy-5-methoxypentyl)piperidin-1-yl)(3- |
| | amino-4-hydroxycyclopentyl)methanone |
| I-72 | 3-(3-aminopyrrolidin-1-yl)-4-(3-((R)-1-hydroxy-5-methoxy-1-(2- |
| | phenoxyphenyl)pentyl)piperidin-1-yl)cyclobut-3-ene-1,2-dione |
| I-73 | 1-(2-(o-tolyloxy)phenyl)-1-(1-(1-(3-aminopyrrolidin-1-yl)-2-nitrovinyl)piperidin-3-yl)- |
| | 5-methoxypentan-1-ol |
| I-74 | (3-(1-(2-(o-tolyloxy)-3,5-difluorophenyl)-1-hydroxy-5-methoxypentyl)piperidin-1- |
| | yl)(3-amino-4-hydroxycyclopentyl)methanone |
| I-75 | 3-(3-aminopiperidin-1-yl)-4-(3-(1-hydroxy-5-methoxy-1-(2- |
| | phenoxyphenyl)pentyl)piperidin-1-yl)cyclobut-3-ene-1,2-dione |
| I-76 | (3-aminopyrrolidin-1-yl)(3-(1-hydroxy-5-methoxy-1-(3′-methylbiphenyl-2- |
| | yl)pentyl)piperidin-1-yl)methanone |
| I-77 | (3-aminopyrrolidin-1-yl)(3-(1-hydroxy-5-methoxy-1-(4′-methylbiphenyl-2- |
| | yl)pentyl)piperidin-1-yl)methanone |
| I-78 | ((1S)-3-amino-4-hydroxycyclopentyl)(3-(1-hydroxy-5-methoxy-1-(2-(pyridin-2- |
| | yl)phenyl)pentyl)piperidin-1-yl)methanone |
| I-79 | (3-amino-4-hydroxypyrrolidin-1-yl)(3-(1-(biphenyl-2-yl)-1-hydroxy-5- |
| | methoxypentyl)piperidin-1-yl)methanone |
| I-80 | (3-amino-4-hydroxypyrrolidin-1-yl)(3-(1-hydroxy-5-methoxy-1-(2-(pyridin-2- |
| | yl)phenyl)pentyl)piperidin-1-yl)methanone |
| I-81 | ((1R,3S)-3-aminocyclopentyl)(3-(1-(4′-fluorobiphenyl-2-yl)-1-hydroxy-5- |
| | methoxypentyl)piperidin-1-yl)methanone |
| I-82 | (2-(aminomethyl)cyclopropyl)(3-(1-(6-fluoro-3′-methylbiphenyl-2-yl)-1-hydroxy-5- |
| | methoxypentyl)piperidin-1-yl)methanone |
| I-83 | (3-aminocyclopentyl)(3-(1-(2′-fluorobiphenyl-2-yl)-1-hydroxy-5- |
| | methoxypentyl)piperidin-1-yl)methanone |
| I-84 | (3-aminocyclopentyl)(3-(1-(3′-fluorobiphenyl-2-yl)-1-hydroxy-5- |
| | methoxypentyl)piperidin-1-yl)methanone |
| I-85 | (3-aminocyclopentyl)(3-(1-(6-fluorobiphenyl-2-yl)-1-hydroxy-5- |
| | methoxypentyl)piperidin-1-yl)methanone |
| I-86 | (3-aminopyrrolidin-1-yl)(3-(1-(2′-fluorobiphenyl-2-yl)-1-hydroxy-5- |
| | methoxypentyl)piperidin-1-yl)methanone |
| I-87 | (3-aminopyrrolidin-1-yl)(3-(1-(3′-fluorobiphenyl-2-yl)-1-hydroxy-5- |
| | methoxypentyl)piperidin-1-yl)methanone |
| I-88 | (3-aminopyrrolidin-1-yl)(3-(1-(4′-fluorobiphenyl-2-yl)-1-hydroxy-5- |
| | methoxypentyl)piperidin-1-yl)methanone |
| I-89 | (3-amino-4-hydroxycyclopentyl)(3-(1-hydroxy-5-methoxy-1-(2-(5-methylfuran-2- |
| | yl)phenyl)pentyl)piperidin-1-yl)methanone |
| I-90 | N-(4-(1-(3-amino-4-hydroxycyclopentanecarbonyl)piperidin-3-yl)-4-(biphenyl-2-yl)-4- |
| | hydroxybutyl)acetamide |
| I-91 | N-(4-(1-(3-amino-4-hydroxypyrrolidine-1-carbonyl)piperidin-3-yl)-4-(biphenyl-2-yl)-4- |
| | hydroxybutyl)acetamide |
| I-92 | (3-amino-4-hydroxycyclopentyl)(3-(1-hydroxy-5-methoxy-1-(2′-methylbiphenyl-2- |
| | yl)pentyl)piperidin-1-yl)methanone |
| I-93 | (3-amino-4-hydroxycyclopentyl)(3-(1-hydroxy-5-methoxy-1-(4′-methylbiphenyl-2- |
| | yl)pentyl)piperidin-1-yl)methanone |
| I-94 | (3-amino-4-hydroxypyrrolidin-1-yl)(3-(1-hydroxy-5-methoxy-1-(2′-methylbiphenyl-2- |
| | yl)pentyl)piperidin-1-yl)methanone |
| I-95 | (3-amino-4-hydroxypyrrolidin-1-yl)(3-(1-hydroxy-5-methoxy-1-(3′-methylbiphenyl-2- |
| | yl)pentyl)piperidin-1-yl)methanone |
| I-96 | (3-amino-4-hydroxypyrrolidin-1-yl)(3-(1-hydroxy-5-methoxy-1-(4′-methylbiphenyl-2- |
| | yl)pentyl)piperidin-1-yl)methanone |
| I-97 | (3-(1-(6-fluoro-3′-methylbiphenyl-2-yl)-1-hydroxy-5-methoxypentyl)piperidin-1-yl)(2- |
| | ((methylamino)methyl)cyclopropyl)methanone |
| I-98 | (3-amino-4-hydroxycyclopentyl)(3-(1-(6-fluoro-3′-methylbiphenyl-2-yl)-5- |
| | methoxypentyl)piperidin-1-yl)methanone |
| I-99 | (2-(aminomethyl)cyclopropyl)(3-(1-(3-fluoro-2-(o-tolyloxy)phenyl)-1-hydroxy-5- |
| | methoxypentyl)piperidin-1-yl)methanone |
| I-100 | (3-amino-4-hydroxycyclopentyl)(2-(1-(6-fluoro-3′-methylbiphenyl-2-yl)-5- |
| | methoxypentyl)morpholino)methanone |
| I-101 | (3-amino-4-hydroxycyclopentyl)(3-(1-(2′-fluorobiphenyl-2-yl)-1-hydroxy-5- |
| | methoxypentyl)piperidin-1-yl)methanone |
| I-102 | (3-amino-4-hydroxycyclopentyl)(3-(1-(3′-fluorobiphenyl-2-yl)-1-hydroxy-5- |
| | methoxypentyl)piperidin-1-yl)methanone |
| I-103 | (3-amino-4-hydroxycyclopentyl)(3-(1-(4′-fluorobiphenyl-2-yl)-1-hydroxy-5- |
| | methoxypentyl)piperidin-1-yl)methanone |
| I-104 | (3-aminocyclopentyl)(3-(1-(2′-chlorobiphenyl-2-yl)-1-hydroxy-5- |
| | methoxypentyl)piperidin-1-yl)methanone |
| I-105 | (3-aminocyclopentyl)(3-(1-(3′-chlorobiphenyl-2-yl)-1-hydroxy-5- |
| | methoxypentyl)piperidin-1-yl)methanone |
| I-106 | (3-aminocyclopentyl)(3-(1-(4′-chlorobiphenyl-2-yl)-1-hydroxy-5- |
| | methoxypentyl)piperidin-1-yl)methanone |
| I-107 | (3-amino-4-hydroxypyrrolidin-1-yl)(2-(1-(6-fluoro-3′-methylbiphenyl-2-yl)-5- |
| | methoxypentyl)morpholino)methanone |
| I-108 | (3-amino-4-hydroxypyrrolidin-1-yl)(3-(1-(2′-fluorobiphenyl-2-yl)-1-hydroxy-5- |
| | methoxypentyl)piperidin-1-yl)methanone |
| I-109 | (3-amino-4-hydroxypyrrolidin-1-yl)(3-(1-(3′-fluorobiphenyl-2-yl)-1-hydroxy-5- |
| | methoxypentyl)piperidin-1-yl)methanone |
| I-110 | (3-amino-4-hydroxypyrrolidin-1-yl)(3-(1-(4′-fluorobiphenyl-2-yl)-1-hydroxy-5- |
| | methoxypentyl)piperidin-1-yl)methanone |
| I-111 | (3-amino-4-hydroxypyrrolidin-1-yl)(3-(1-(6-fluorobiphenyl-2-yl)-1-hydroxy-5- |
| | methoxypentyl)piperidin-1-yl)methanone |
| I-112 | (3-aminopyrrolidin-1-yl)(3-(1-(2′-chlorobiphenyl-2-yl)-1-hydroxy-5- |
| | methoxypentyl)piperidin-1-yl)methanone |
| I-113 | (3-aminopyrrolidin-1-yl)(3-(1-(3′-chlorobiphenyl-2-yl)-1-hydroxy-5- |
| | methoxypentyl)piperidin-1-yl)methanone |
| I-114 | (3-aminopyrrolidin-1-yl)(3-(1-(4′-chlorobiphenyl-2-yl)-1-hydroxy-5- |
| | methoxypentyl)piperidin-1-yl)methanone |
| I-115 | (3-amino-4-hydroxycyclopentyl)(3-(1-(2-cyclohexenyl-3-fluorophenyl)-1-hydroxy-5- |
| | methoxypentyl)piperidin-1-yl)methanone |
| I-116 | (3-amino-4-hydroxycyclopentyl)(3-(1-(3-fluoro-2-(piperidin-1-yl)phenyl)-1-hydroxy-5- |
| | methoxypentyl)piperidin-1-yl)methanone |
| I-117 | N-(2-((1-(3-amino-4-hydroxycyclopentanecarbonyl)piperidin-3-yl)(6-fluoro-3′- |
| | methylbiphenyl-2-yl)methoxy)ethyl)acetamide |
| I-118 | (3-amino-4-hydroxypyrrolidin-1-yl)(3-(1-hydroxy-5-methoxy-1-(2-(o- |
| | tolyloxy)phenyl)pentyl)piperidin-1-yl)methanone |
| I-119 | (3-amino-3-methylpyrrolidin-1-yl)(3-(1-(6-fluoro-3′-methylbiphenyl-2-yl)-1-hydroxy-5- |
| | methoxypentyl)piperidin-1-yl)methanone |
| I-120 | (3-amino-4-hydroxycyclopentyl)(2-(1-hydroxy-5-methoxy-1-(2-(o- |
| | tolyloxy)phenyl)pentyl)morpholino)methanone |
| I-121 | ((R)-3-((S)-1-(3-fluoro-2-(o-tolyloxy)phenyl)-1-hydroxy-5-methoxypentyl)piperidin-1- |
| | yl)((1RS,2RS)-2-((methylamino)methyl)cyclopropyl)methanone |
| I-122 | (3-amino-4-hydroxycyclopentyl)(3-(1-(2′-fluoro-5′-methylbiphenyl-2-yl)-1-hydroxy-5- |
| | methoxypentyl)piperidin-1-yl)methanone |
| I-123 | (3-amino-4-hydroxycyclopentyl)(3-(1-(4-fluoro-3′-methylbiphenyl-2-yl)-1-hydroxy-5- |
| | methoxypentyl)piperidin-1-yl)methanone |
| I-124 | (3-amino-4-hydroxycyclopentyl)(3-(1-(5-fluoro-3′-methylbiphenyl-2-yl)-1-hydroxy-5- |
| | methoxypentyl)piperidin-1-yl)methanone |
| I-125 | (3-amino-4-hydroxycyclopentyl)(3-(1-(5-fluoro-3′-methylbiphenyl-2-yl)-1-hydroxy-5- |
| | methoxypentyl)piperidin-1-yl)methanone |
| I-126 | (3-amino-4-hydroxycyclopentyl)(3-(1-(6-fluoro-3′-methylbiphenyl-2-yl)-1-hydroxy-5- |
| | methoxypentyl)piperidin-1-yl)methanone |
| I-127 | (3-amino-4-hydroxypyrrolidin-1-yl)(2-(1-hydroxy-5-methoxy-1-(2-(o- |
| | tolyloxy)phenyl)pentyl)morpholino)methanone |
| I-128 | (3-amino-4-hydroxypyrrolidin-1-yl)(3-(1-(2′-fluoro-5′-methylbiphenyl-2-yl)-1-hydroxy- |
| | 5-methoxypentyl)piperidin-1-yl)methanone |
| I-129 | (3-amino-4-hydroxypyrrolidin-1-yl)(3-(1-(6-fluoro-3′-methylbiphenyl-2-yl)-1-hydroxy- |
| | 5-methoxypentyl)piperidin-1-yl)methanone |
| I-130 | (3-amino-4-hydroxycyclopentyl)(2-(1-(6-fluoro-3′-methylbiphenyl-2-yl)-1-hydroxy-5- |
| | methoxypentyl)morpholino)methanone |
| I-131 | (3-amino-4-hydroxycyclopentyl)(3-(1-(3′-chlorobiphenyl-2-yl)-1-hydroxy-5- |
| | methoxypentyl)piperidin-1-yl)methanone |
| I-132 | (3-amino-4-hydroxycyclopentyl)(3-(1-(4′-chlorobiphenyl-2-yl)-1-hydroxy-5- |
| | methoxypentyl)piperidin-1-yl)methanone |
| I-133 | (3-amino-4-hydroxypyrrolidin-1-yl)(3-(1-(2-(3-fluorophenoxy)phenyl)-1-hydroxy-5- |
| | methoxypentyl)piperidin-1-yl)methanone |
| I-134 | (3-amino-4-hydroxypyrrolidin-1-yl)(3-(1-(2′-chlorobiphenyl-2-yl)-1-hydroxy-5- |
| | methoxypentyl)piperidin-1-yl)methanone |
| I-135 | (3-amino-4-hydroxypyrrolidin-1-yl)(3-(1-(3′-chlorobiphenyl-2-yl)-1-hydroxy-5- |
| | methoxypentyl)piperidin-1-yl)methanone |
| I-136 | (3-amino-4-hydroxypyrrolidin-1-yl)(3-(1-(4′-chlorobiphenyl-2-yl)-1-hydroxy-5- |
| | methoxypentyl)piperidin-1-yl)methanone |
| I-137 | (3-amino-4-hydroxycyclopentyl)(3-(1-(3′,6-difluorobiphenyl-2-yl)-1-hydroxy-5- |
| | methoxypentyl)piperidin-1-yl)methanone |
| I-138 | (3-amino-4-hydroxypyrrolidin-1-yl)(3-(1-(2′,3′-difluorobiphenyl-2-yl)-1-hydroxy-5- |
| | methoxypentyl)piperidin-1-yl)methanone |
| I-139 | (3-amino-4-hydroxypyrrolidin-1-yl)(3-(1-(3′,6-difluorobiphenyl-2-yl)-1-hydroxy-5- |
| | methoxypentyl)piperidin-1-yl)methanone |
| I-140 | N-(4-(1-(3-amino-4-hydroxycyclopentanecarbonyl)piperidin-3-yl)-4-(2′-fluoro-5′- |
| | methylbiphenyl-2-yl)-4-hydroxybutyl)acetamide |
| I-141 | N-(4-(1-(3-amino-4-hydroxycyclopentanecarbonyl)piperidin-3-yl)-4-(6-fluoro-3′- |
| | methylbiphenyl-2-yl)-4-hydroxybutyl)acetamide |
| I-142 | (3-amino-3-(hydroxymethyl)pyrrolidin-1-yl)(3-(1-hydroxy-5-methoxy-1-(2-(o- |
| | tolyloxy)phenyl)pentyl)piperidin-1-yl)methanone |
| I-143 | (3-amino-4-hydroxypyrrolidin-1-yl)(3-(1-(2-(2-ethylphenoxy)phenyl)-1-hydroxy-5- |
| | methoxypentyl)piperidin-1-yl)methanone |
| I-144 | (3-amino-4-hydroxypyrrolidin-1-yl)(3-(5-ethoxy-1-hydroxy-1-(2-(o- |
| | tolyloxy)phenyl)pentyl)piperidin-1-yl)methanone |
| I-145 | N-(4-(1-(3-amino-4-hydroxypyrrolidine-1-carbonyl)piperidin-3-yl)-4-(6-fluoro-3′- |
| | methylbiphenyl-2-yl)-4-hydroxybutyl)acetamide |
| I-146 | (3-amino-4-hydroxycyclopentyl)(3-(1-(6-fluoro-3′,5′-dimethylbiphenyl-2-yl)-1- |
| | hydroxy-5-methoxypentyl)piperidin-1-yl)methanone |
| I-147 | (3-(1-(6-fluoro-3′-methylbiphenyl-2-yl)-1-hydroxy-5-methoxypentyl)piperidin-1-yl)(3- |
| | hydroxy-4-(methylamino)pyrrolidin-1-yl)methanone |
| I-148 | (3-amino-3-methylpyrrolidin-1-yl)(3-(1-(3-fluoro-2-(o-tolyloxy)phenyl)-1-hydroxy-5- |
| | methoxypentyl)piperidin-1-yl)methanone |
| I-149 | (3-amino-4-hydroxypyrrolidin-1-yl)(3-(1-(6-fluoro-3′,5′-dimethylbiphenyl-2-yl)-1- |
| | hydroxy-5-methoxypentyl)piperidin-1-yl)methanone |
| I-150 | (3-amino-4-hydroxycyclopentyl)(3-(1-(6-chloro-3′-methylbiphenyl-2-yl)-1-hydroxy-5- |
| | methoxypentyl)piperidin-1-yl)methanone |
| I-151 | (3-amino-4-hydroxypyrrolidin-1-yl)(3-(1-(3-fluoro-2-(o-tolyloxy)phenyl)-1-hydroxy-5- |
| | methoxypentyl)piperidin-1-yl)methanone |
| I-152 | (3-amino-4-hydroxypyrrolidin-1-yl)(3-(1-(6-chloro-3′-methylbiphenyl-2-yl)-1-hydroxy- |
| | 5-methoxypentyl)piperidin-1-yl)methanone |
| I-153 | (3-amino-4-hydroxycyclopentyl)(3-(1-(2′,6-difluoro-5′-methylbiphenyl-2-yl)-1- |
| | hydroxy-5-methoxypentyl)piperidin-1-yl)methanone |
| I-154 | (3-amino-4-hydroxycyclopentyl)(2-(1-(6-chloro-3′-methylbiphenyl-2-yl)-1-hydroxy-5- |
| | methoxypentyl)morpholino)methanone |
| I-155 | (3-amino-4-hydroxycyclopentyl)(2-(1-(6-chloro-3′-methylbiphenyl-2-yl)-1-hydroxy-5- |
| | methoxypentyl)morpholino)methanone |
| I-156 | (3-amino-4-hydroxypyrrolidin-1-yl)(3-(1-(2′,6-difluoro-5′-methylbiphenyl-2-yl)-1- |
| | hydroxy-5-methoxypentyl)piperidin-1-yl)methanone |
| I-157 | (3-amino-4-hydroxypyrrolidin-1-yl)(2-(1-(6-chloro-3′-methylbiphenyl-2-yl)-1-hydroxy- |
| | 5-methoxypentyl)morpholino)methanone |
| I-158 | (3-amino-4-hydroxycyclopentyl)(3-(1-(3′-chloro-6-fluorobiphenyl-2-yl)-1-hydroxy-5- |
| | methoxypentyl)piperidin-1-yl)methanone |
| I-159 | (3-amino-4-hydroxycyclopentyl)(3-(1-(6-chloro-3′-fluorobiphenyl-2-yl)-1-hydroxy-5- |
| | methoxypentyl)piperidin-1-yl)methanone |
| I-160 | (3-amino-4-hydroxypyrrolidin-1-yl)(3-(1-(2-fluoro-5-(4-fluorophenoxy)phenyl)-1- |
| | hydroxy-5-methoxypentyl)piperidin-1-yl)methanone |
| I-161 | (3-amino-4-hydroxypyrrolidin-1-yl)(3-(1-(3′-chloro-6-fluorobiphenyl-2-yl)-1-hydroxy- |
| | 5-methoxypentyl)piperidin-1-yl)methanone |
| I-162 | (3-amino-4-hydroxypyrrolidin-1-yl)(3-(1-(6-chloro-3′-fluorobiphenyl-2-yl)-1-hydroxy- |
| | 5-methoxypentyl)piperidin-1-yl)methanone |
| I-163 | methyl 4-(1-(3-amino-4-hydroxycyclopentanecarbonyl)piperidin-3-yl)-4-(6-fluoro-3′- |
| | methylbiphenyl-2-yl)-4-hydroxybutylcarbamate |
| I-164 | N-(4-(1-(3-amino-4-hydroxycyclopentanecarbonyl)piperidin-3-yl)-4-(6-chloro-3′- |
| | methylbiphenyl-2-yl)-4-hydroxybutyl)acetamide |
| I-165 | methyl 4-(1-(3-amino-4-hydroxypyrrolidine-1-carbonyl)piperidin-3-yl)-4-(6-fluoro-3′- |
| | methylbiphenyl-2-yl)-4-hydroxybutylcarbamate |
| I-166 | N-(4-(1-(3-amino-4-hydroxypyrrolidine-1-carbonyl)piperidin-3-yl)-4-(6-chloro-3′- |
| | methylbiphenyl-2-yl)-4-hydroxybutyl)acetamide |
| I-167 | N-(4-(1-(3-amino-4-hydroxycyclopentanecarbonyl)piperidin-3-yl)-4-(2′,6-difluoro-5′- |
| | methylbiphenyl-2-yl)-4-hydroxybutyl)acetamide |
| I-168 | (3-(1-(3-fluoro-2-(o-tolyloxy)phenyl)-1-hydroxy-5-methoxypentyl)piperidin-1-yl)(3- |
| | hydroxy-4-(methylamino)pyrrolidin-1-yl)methanone |
| I-169 | (3-amino-4-hydroxycyclopentyl)(3-(1-hydroxy-5-methoxy-1-(3′- |
| | (trifluoromethyl)biphenyl-2-yl)pentyl)piperidin-1-yl)methanone |
| I-170 | (3-amino-4-hydroxycyclopentyl)(3-(1-(3′,6-dichlorobiphenyl-2-yl)-1-hydroxy-5- |
| | methoxypentyl)piperidin-1-yl)methanone |
| I-171 | (3-amino-4-hydroxypyrrolidin-1-yl)(3-(1-hydroxy-5-methoxy-1-(3′- |
| | (trifluoromethyl)biphenyl-2-yl)pentyl)piperidin-1-yl)methanone |
| I-172 | (3-amino-4-hydroxypyrrolidin-1-yl)(3-(1-(3′,6-dichlorobiphenyl-2-yl)-1-hydroxy-5- |
| | methoxypentyl)piperidin-1-yl)methanone |
| I-173 | (3-amino-4-hydroxycyclopentyl)(3-(1-(3′-chloro-2′,6-difluorobiphenyl-2-yl)-1-hydroxy- |
| | 5-methoxypentyl)piperidin-1-yl)methanone |
| I-174 | (3-amino-4-hydroxycyclopentyl)(2-(1-(6-fluoro-3′-(trifluoromethoxy)biphenyl-2-yl)-1- |
| | hydroxy-5-methoxypentyl)morpholino)methanone |
| I-175 | methyl 4-(1-(3-amino-4-hydroxycyclopentanecarbonyl)piperidin-3-yl)-4-(6-chloro-3′- |
| | methylbiphenyl-2-yl)-4-hydroxybutylcarbamate |
| I-176 | methyl 4-(1-(3-amino-4-hydroxypyrrolidine-1-carbonyl)piperidin-3-yl)-4-(6-chloro-3′- |
| | methylbiphenyl-2-yl)-4-hydroxybutylcarbamate and |
| I-177 | methyl 2-((1-(3-amino-4-hydroxycyclopentanecarbonyl)piperidin-3-yl)(6-fluoro-3′- |
| | methylbiphenyl-2-yl)methoxy)ethylcarbamate |
| I-178 | (3-amino-4-hydroxypyrrolidin-1-yl)(3-(1-(6-fluoro-3′-methylbiphenyl-2-yl)-5- |
| | methoxypentyl)phenyl)methanone |
| I-178 | (3-amino-4-hydroxypyrrolidin-1-yl)(3-(1-(6-fluoro-3′-methylbiphenyl-2-yl)-5- |
| | methoxypentyl)phenyl)methanone |
| I-179 | (3-amino-4-hydroxycyclopentyl)(3-((6-fluoro-3′-methylbiphenyl-2-yl)(3- |
| | methoxypropoxy)methyl)piperidin-1-yl)methanone |
| I-180 | (3-amino-4-hydroxycyclopentyl)(2-((6-fluoro-3′-methylbiphenyl-2-yl)(3- |
| | methoxypropoxy)methyl)morpholino)methanone |
| I-181 | (3-amino-4-hydroxypyrrolidin-1-yl)(3-(1-(6-fluoro-3′-methylbiphenyl-2-yl)-1-hydroxy- |
| | 5-methoxypentyl)phenyl)methanone |
| I-182 | N-(4-(1-(3-amino-4-hydroxycyclopentanecarbonyl)piperidin-3-yl)-4-(6-fluoro-3′- |
| | methylbiphenyl-2-yl)butyl)acetamide |
| I-183 | (3-(1-(6-fluoro-3′-methylbiphenyl-2-yl)-1-hydroxy-5-methoxypentyl)piperidin-1-yl)(2- |
| | methyl-3-((methylamino)methyl)cyclopropyl)methanone |
| I-184 | (3-(1-(6-fluoro-3′-methylbiphenyl-2-yl)-1-hydroxy-5-methoxypentyl)piperidin-1-yl)(1- |
| | methyl-2-((methylamino)methyl)cyclopropyl)methanone |
| I-185 | (3-(1-(6-fluoro-3′-methylbiphenyl-2-yl)-1-hydroxy-5-methoxypentyl)piperidin-1-yl)(2- |
| | methyl-2-((methylamino)methyl)cyclopropyl)methanone |
| I-186 | N-(2-((4-(3-amino-4-hydroxycyclopentanecarbonyl)morpholin-2-yl)(6-fluoro-3′- |
| | methylbiphenyl-2-yl)methoxy)ethyl)acetamide |
| I-187 | (3-amino-4-hydroxypyrrolidin-1-yl)(2-(1-(6-fluoro-3′-methylbiphenyl-2-yl)-1-hydroxy- |
| | 5-methoxypentyl)morpholino)methanone |
| I-188 | 6-(1-(1-(3-amino-4-hydroxycyclopentanecarbonyl)piperidin-3-yl)-1-hydroxy-5- |
| | methoxypentyl)-3′-methylbiphenyl-3-carbonitrile |
| I-189 | methyl 4-(1-(3-amino-4-hydroxycyclopentanecarbonyl)piperidin-3-yl)-4-(6-fluoro-3′- |
| | methylbiphenyl-2-yl)butylcarbamate |
| I-190 | (3-amino-4-hydroxycyclopentyl)(2-(1-hydroxy-5-methoxy-1-(3-methoxy-3′- |
| | methylbiphenyl-2-yl)pentyl)morpholino)methanone |
| I-191 | (3-amino-4-hydroxycyclopentyl)(2-(1-(3′-ethyl-6-fluorobiphenyl-2-yl)-1-hydroxy-5- |
| | methoxypentyl)morpholino)methanone |
| I-192 | methyl 2-((4-(3-amino-4-hydroxycyclopentanecarbonyl)morpholin-2-yl)(6-fluoro-3′- |
| | methylbiphenyl-2-yl)methoxy)ethylcarbamate |
| I-193 | (3-amino-4-hydroxycyclopentyl)(2-(1-(6-fluoro-3′-methoxybiphenyl-2-yl)-1-hydroxy-5- |
| | methoxypentyl)morpholino)methanone |
| I-194 | (3-amino-4-hydroxycyclopentyl)(2-(1-(3′-chloro-6-fluorobiphenyl-2-yl)-1-hydroxy-5- |
| | methoxypentyl)morpholino)methanone |
| I-195 | (3-amino-4-hydroxycyclopentyl)(2-(1-(3′-cyclopropyl-6-fluorobiphenyl-2-yl)-1- |
| | hydroxy-5-methoxypentyl)morpholino)methanone |
| I-196 | (3-amino-4-hydroxycyclopentyl)(2-(1-(6-chloro-3′-ethylbiphenyl-2-yl)-1-hydroxy-5- |
| | methoxypentyl)morpholino)methanone |
| I-197 | (3-amino-4-hydroxycyclopentyl)(2-(1-(6-chloro-3′,4′-dimethylbiphenyl-2-yl)-1- |
| | hydroxy-5-methoxypentyl)morpholino)methanone |
| I-198 | (3-amino-4-hydroxycyclopentyl)(2-(1-(3′-ethoxy-6-fluorobiphenyl-2-yl)-1-hydroxy-5- |
| | methoxypentyl)morpholino)methanone |
| I-199 | (3-amino-4-hydroxycyclopentyl)(2-(1-(6-fluoro-3-methoxy-3′-methylbiphenyl-2-yl)-1- |
| | hydroxy-5-methoxypentyl)morpholino)methanone |
| I-200 | (3-amino-4-hydroxycyclopentyl)(2-(1-(6-chloro-3′-methoxybiphenyl-2-yl)-1-hydroxy- |
| | 5-methoxypentyl)morpholino)methanone |
| I-201 | (3-amino-4-hydroxycyclopentyl)(2-(1-(6-fluoro-3′-(methylthio)biphenyl-2-yl)-1- |
| | hydroxy-5-methoxypentyl)morpholino)methanone |
| I-202 | (3-amino-4-hydroxycyclopentyl)(2-(1-(3′,6-dichlorobiphenyl-2-yl)-1-hydroxy-5- |
| | methoxypentyl)morpholino)methanone |
| I-203 | (3-amino-4-hydroxycyclopentyl)(2-(1-(6-chloro-3′-isopropylbiphenyl-2-yl)-1-hydroxy- |
| | 5-methoxypentyl)morpholino)methanone |
| I-204 | (3-amino-4-hydroxycyclopentyl)(2-(1-(6-chloro-3′-(methylthio)biphenyl-2-yl)-1- |
| | hydroxy-5-methoxypentyl)morpholino)methanone |
| I-205 | (3-amino-4-hydroxycyclopentyl)(2-(1-(6-fluoro-3′-(trifluoromethyl)biphenyl-2-yl)-1- |
| | hydroxy-5-methoxypentyl)morpholino)methanone |
| I-206 | ((1S,3R,4S)-3-amino-4-hydroxycyclopentyl)((R)-3-((S)-1-hydroxy-5-methoxy-1-(2- |
| | (trimethylsilyl)benzofuran-7-yl)pentyl)piperidin-1-yl)methanone |
| I-207 | ((1S,3R,4S)-3-amino-4-hydroxycyclopentyl)((R)-3-((S)-1-hydroxy-5-methoxy-1-(2- |
| | (trimethylsilyl)benzo[b]thiophen-4-yl)pentyl)piperidin-1-yl)methanone |
| I-208 | ((1S,3R,4S)-3-amino-4-hydroxycyclopentyl)((R)-3-((S)-1-hydroxy-5-methoxy-1- |
| | (spiro[benzo[d][1,3]dioxole-2,1′-cyclohexane]-4-yl)pentyl)piperidin-1-yl)methanone |
| I-209 | ((1S,3R,4S)-3-amino-4-hydroxycyclopentyl)((R)-3-((S)-1,5-dimethoxy-1- |
| | (spiro[benzo[d][1,3]dioxole-2,1′-cyclohexane]-4-yl)pentyl)piperidin-1-yl)methanone |
| I-210 | ((1S,3R,4S)-3-amino-4-hydroxycyclopentyl)((R)-3-((S)-1,5-dimethoxy-1- |
| | (spiro[benzo[d][1,3]dioxole-2,1′-cyclopentane]-4-yl)pentyl)piperidin-1-yl)methanone |
| I-211 | ((1S,3R,4S)-3-amino-4-hydroxycyclopentyl)((R)-3-((S)-1-(2-tert-butylbenzo[d]oxazol- |
| | 7-yl)-1-hydroxy-5-methoxypentyl)piperidin-1-yl)methanone |
| I-212 | ((1S,3R,4S)-3-amino-4-hydroxycyclopentyl)((R)-3-((S)-1-(2-ethylbenzofuran-7-yl)-1- |
| | hydroxy-5-methoxypentyl)piperidin-1-yl)methanone |
| I-213 | ((1S,3R,4S)-3-amino-4-hydroxycyclopentyl)((R)-3-((S)-1-hydroxy-1-(2- |
| | isobutylbenzofuran-7-yl)-5-methoxypentyl)piperidin-1-yl)methanone |
| I-214 | ((1S,3R,4S)-3-amino-4-hydroxycyclopentyl)((R)-3-((S)-1-hydroxy-5-methoxy-1-(2- |
| | (trimethylsilyl)benzofuran-4-yl)pentyl)piperidin-1-yl)methanone |
| I-215 | ((1S,3R,4S)-3-amino-4-hydroxycyclopentyl)((R)-3-((S)-1-(2-tert-butylbenzofuran-7-yl)- |
| | 1-hydroxy-5-methoxypentyl)piperidin-1-yl)methanone |
| I-216 | ((3R,4S)-3-amino-4-hydroxypyrrolidin-1-yl)((R)-3-((S)-1-(2-tert-butylbenzofuran-7-yl)- |
| | 1-hydroxy-5-methoxypentyl)piperidin-1-yl)methanone |
| I-217 | methyl 4-(1-(3-aminocyclopentanecarbonyl)piperidin-3-yl)-4-hydroxy-4-(2-(pyridin-4- |
| | yl)phenyl)butylcarbamate |
| I-218 | 2-((4-(3-aminocyclopentanecarbonyl)morpholin-2-yl)(6-fluoro-3′-methylbiphenyl-2- |
| | yl)methoxy)-N-ethylacetamide |
| I-219 | N-(4-(1-(3-aminocyclopentanecarbonyl)piperidin-3-yl)-4-hydroxy-4-(2-(o- |
| | tolyloxy)phenyl)butyl)acetamide |
| I-219 | N-(4-(1-(3-aminocyclopentanecarbonyl)piperidin-3-yl)-4-hydroxy-4-(2-(o- |
| | tolyloxy)phenyl)butyl)acetamide |
| I-220 | (3-aminocyclopentyl)(3-(1-(2-(2,6-dimethylphenoxy)phenyl)-1-hydroxy-5- |
| | methoxypentyl)piperidin-1-yl)methanone |
| I-221 | methyl 4-(1-(3-amino-4-hydroxycyclopentanecarbonyl)piperidin-3-yl)-4-hydroxy-4-(2- |
| | (pyridin-3-yl)phenyl)butylcarbamate |
| I-222 | (3-amino-4-hydroxycyclopentyl)(2-(1-(6-chloro-3′-ethylbiphenyl-2-yl)-1-hydroxypent- |
| | 4-enyl)morpholino)methanone |
| I-223 | methyl 2-((4-(3-aminocyclopentanecarbonyl)morpholin-2-yl)(6-fluoro-3′- |
| | methylbiphenyl-2-yl)methoxy)ethylcarbamate |
| I-224 | 2-((4-(3-amino-4-hydroxycyclopentanecarbonyl)morpholin-2-yl)(6-fluoro-3′- |
| | methylbiphenyl-2-yl)methoxy)-N-ethylacetamide |
| I-225 | (3-aminocyclopentyl)(2-(1-(4′,6-difluoro-3′-methylbiphenyl-2-yl)-1-hydroxy-5- |
| | methoxypentyl)morpholino)methanone |
| I-226 | (3-amino-4-hydroxycyclopentyl)(2-(1-(3-chloro-2-(pyridin-3-yl)phenyl)-1-hydroxy-5- |
| | methoxypentyl)morpholino)methanone |
| I-226 | (3-amino-4-hydroxycyclopentyl)(2-(1-(3-chloro-2-(pyridin-3-yl)phenyl)-1-hydroxy-5- |
| | methoxypentyl)morpholino)methanone |
| I-227 | (3-amino-4-hydroxycyclopentyl)(2-(1-(3-chloro-2-(3-methyl-1,2,4-oxadiazol-5- |
| | yl)phenyl)-1-hydroxy-5-methoxypentyl)morpholino)methanone |
| I-228 | methyl 4-(1-(3-aminocyclopentanecarbonyl)piperidin-3-yl)-4-hydroxy-4-(2-(o- |
| | tolyloxy)phenyl)butylcarbamate |
| I-229 | N-(4-(1-(3-amino-4-hydroxycyclopentanecarbonyl)piperidin-3-yl)-4-hydroxy-4-(2-(o- |
| | tolyloxy)phenyl)butyl)acetamide |
| I-230 | (3-amino-4-hydroxycyclopentyl)(3-(1-(2-(2,6-dimethylphenoxy)phenyl)-1-hydroxy-5- |
| | methoxypentyl)piperidin-1-yl)methanone |
| I-231 | (3-aminocyclopentyl)(3-(1-(6-fluoro-3′-methoxy-5′-methylbiphenyl-2-yl)-1-hydroxy-5- |
| | methoxypentyl)piperidin-1-yl)methanone |
| I-232 | (3-aminocyclopentyl)(3-(1-(6-chloro-3′-ethylbiphenyl-2-yl)-1-hydroxy-5- |
| | methoxypentyl)piperidin-1-yl)methanone |
| I-233 | (3-aminocyclopentyl)(3-(1-(3-chloro-2-(2-methylbenzyl)phenyl)-1-hydroxy-5- |
| | methoxypentyl)piperidin-1-yl)methanone |
| I-234 | (3-aminocyclopentyl)(3-(1-(3-chloro-2-(3-methylbenzyl)phenyl)-1-hydroxy-5- |
| | methoxypentyl)piperidin-1-yl)methanone |
| I-235 | (3-aminocyclopentyl)(2-(1-(6-fluoro-3′-methoxy-5′-methylbiphenyl-2-yl)-1-hydroxy-5- |
| | methoxypentyl)morpholino)methanone |
| I-236 | (3-aminocyclopentyl)(2-(1-(6-chloro-3′-ethylbiphenyl-2-yl)-1-hydroxy-5- |
| | methoxypentyl)morpholino)methanone |
| I-237 | (3-aminocyclopentyl)(3-((R)-1-(3-chloro-2-(o-tolyloxy)phenyl)-1-hydroxy-5- |
| | methoxypentyl)piperidin-1-yl)methanone |
| I-238 | (3-aminocyclopentyl)(3-(1-(2-(2-chloro-6-methylphenoxy)phenyl)-1-hydroxy-5- |
| | methoxypentyl)piperidin-1-yl)methanone |
| I-239 | N-((S)-4-((R)-1-(4-(aminomethyl)benzoyl)piperidin-3-yl)-4-hydroxy-4-(2-(o- |
| | tolyloxy)phenyl)butyl)acetamide |
| I-239 | N-((S)-4-((R)-1-(4-(aminomethyl)benzoyl)piperidin-3-yl)-4-hydroxy-4-(2-(o- |
| | tolyloxy)phenyl)butyl)acetamide |
| I-240 | (3-amino-4-hydroxycyclopentyl)(2-(1-(4′,6-difluoro-3′-methylbiphenyl-2-yl)-1- |
| | hydroxy-5-methoxypentyl)morpholino)methanone |
| I-240 | (3-amino-4-hydroxycyclopentyl)(2-(1-(4′,6-difluoro-3′-methylbiphenyl-2-yl)-1- |
| | hydroxy-5-methoxypentyl)morpholino)methanone |
| I-241 | (4-(aminomethyl)phenyl)(3-(1-(3′-ethyl-6-fluorobiphenyl-2-yl)-1-hydroxy-5- |
| | methoxypentyl)piperidin-1-yl)methanone |
| I-242 | (3-(aminomethyl)phenyl)(4-(1-(3′-ethyl-6-fluorobiphenyl-2-yl)-1-hydroxy-5- |
| | methoxypentyl)piperidin-1-yl)methanone |
| I-243 | (4-(aminomethyl)phenyl)(2-(1-(4′,6-difluoro-3′-methylbiphenyl-2-yl)-1-hydroxy-5- |
| | methoxypentyl)morpholino)methanone |
| I-244 | methyl 4-(1-(3-amino-4-hydroxycyclopentanecarbonyl)piperidin-3-yl)-4-hydroxy-4-(2- |
| | (o-tolyloxy)phenyl)butylcarbamate |
| I-244 | methyl 4-(1-(3-amino-4-hydroxycyclopentanecarbonyl)piperidin-3-yl)-4-hydroxy-4-(2- |
| | (o-tolyloxy)phenyl)butylcarbamate |
| I-245 | methyl 4-(1-(2-aminocyclopentanecarbonyl)piperidin-3-yl)-4-(3′-ethyl-6- |
| | fluorobiphenyl-2-yl)-4-hydroxybutylcarbamate |
| I-246 | methyl 4-(1-(3-aminocyclopentanecarbonyl)piperidin-3-yl)-4-(3′-ethyl-6- |
| | fluorobiphenyl-2-yl)-4-hydroxybutylcarbamate |
| I-247 | (4-aminocyclohexyl)(3-(1-(6-chloro-3′-ethylbiphenyl-2-yl)-1-hydroxy-5- |
| | methoxypentyl)piperidin-1-yl)methanone |
| I-248 | methyl 4-(3′-ethyl-6-fluorobiphenyl-2-yl)-4-hydroxy-4-(1-(4-hydroxypyrrolidine-2- |
| | carbonyl)piperidin-3-yl)butylcarbamate |
| I-249 | (3-amino-4-hydroxycyclopentyl)(3-(1-(6-fluoro-3′-methoxy-5′-methylbiphenyl-2-yl)-1- |
| | hydroxy-5-methoxypentyl)piperidin-1-yl)methanone |
| I-250 | (3-amino-4-hydroxycyclopentyl)(3-(1-(6-chloro-3′-ethylbiphenyl-2-yl)-1-hydroxy-5- |
| | methoxypentyl)piperidin-1-yl)methanone |
| I-251 | (3-aminocyclopentyl)(3-(1-(3-chloro-2-(2-ethylphenoxy)phenyl)-1-hydroxy-5- |
| | methoxypentyl)piperidin-1-yl)methanone |
| I-252 | (3-aminocyclopentyl)(3-(1-(3-chloro-2-(3-ethylphenoxy)phenyl)-1-hydroxy-5- |
| | methoxypentyl)piperidin-1-yl)methanone |
| I-253 | (3-amino-4-hydroxycyclopentyl)(3-(1-(3-chloro-2-(2-methylbenzyl)phenyl)-1-hydroxy- |
| | 5-methoxypentyl)piperidin-1-yl)methanone |
| I-254 | (3-amino-4-hydroxycyclopentyl)(3-(1-(3-chloro-2-(3-methylbenzyl)phenyl)-1-hydroxy- |
| | 5-methoxypentyl)piperidin-1-yl)methanone |
| I-255 | (3-amino-4-hydroxycyclopentyl)(2-(1-(6-fluoro-3′-methoxy-5′-methylbiphenyl-2-yl)-1- |
| | hydroxy-5-methoxypentyl)morpholino)methanone |
| I-256 | (3-amino-4-hydroxycyclopentyl)(3-(1-(3-chloro-2-(o-tolyloxy)phenyl)-1-hydroxy-5- |
| | methoxypentyl)piperidin-1-yl)methanone |
| I-257 | (3-amino-4-hydroxycyclopentyl)(2-(1-(6-chloro-3′-ethylbiphenyl-2-yl)-1-hydroxy-5- |
| | methoxypentyl)morpholino)methanone |
| I-257 | (3-amino-4-hydroxycyclopentyl)(2-(1-(6-chloro-3′-ethylbiphenyl-2-yl)-1-hydroxy-5- |
| | methoxypentyl)morpholino)methanone |
| I-258 | methyl 4-(1-(4-(aminomethyl)benzoyl)piperidin-3-yl)-4-hydroxy-4-(2-(o- |
| | tolyloxy)phenyl)butylcarbamate |
| I-258 | methyl 4-(1-(4-(aminomethyl)benzoyl)piperidin-3-yl)-4-hydroxy-4-(2-(o- |
| | tolyloxy)phenyl)butylcarbamate |
| I-259 | (4-(2-aminoethyl)phenyl)(4-(1-(3′-ethyl-6-fluorobiphenyl-2-yl)-1-hydroxy-5- |
| | methoxypentyl)piperidin-1-yl)methanone |
| I-260 | (4-(aminomethyl)phenyl)(3-(1-(6-fluoro-3′-methoxy-5′-methylbiphenyl-2-yl)-1- |
| | hydroxy-5-methoxypentyl)piperidin-1-yl)methanone |
| I-261 | (3-amino-4-hydroxycyclopentyl)(2-(1-(6-chloro-2′-fluoro-5′-methylbiphenyl-2-yl)-1- |
| | hydroxy-5-methoxypentyl)morpholino)methanone |
| I-262 | (4-(aminomethyl)phenyl)(3-(1-(6-chloro-3′-ethylbiphenyl-2-yl)-1-hydroxy-5- |
| | methoxypentyl)piperidin-1-yl)methanone |
| I-263 | (4-(aminomethyl)phenyl)(3-(1-(3-chloro-2-(2-methylbenzyl)phenyl)-1-hydroxy-5- |
| | methoxypentyl)piperidin-1-yl)methanone |
| I-264 | (4-(aminomethyl)phenyl)(3-(1-(3-chloro-2-(3-methylbenzyl)phenyl)-1-hydroxy-5- |
| | methoxypentyl)piperidin-1-yl)methanone |
| I-265 | (3-aminocyclopentyl)(3-(1-(3-chloro-2-(quinolin-3-yl)phenyl)-1-hydroxy-5- |
| | methoxypentyl)piperidin-1-yl)methanone |
| I-265 | (3-aminocyclopentyl)(3-(1-(3-chloro-2-(quinolin-3-yl)phenyl)-1-hydroxy-5- |
| | methoxypentyl)piperidin-1-yl)methanone |
| I-266 | (6-(aminomethyl)pyridin-3-yl)(3-(1-(6-chloro-3′-ethylbiphenyl-2-yl)-1-hydroxy-5- |
| | methoxypentyl)piperidin-1-yl)methanone |
| I-267 | (4-(aminomethyl)phenyl)(2-(1-(6-fluoro-3′-methoxy-5′-methylbiphenyl-2-yl)-1- |
| | hydroxy-5-methoxypentyl)morpholino)methanone |
| I-268 | (3-(aminomethyl)phenyl)(2-(1-(6-chloro-3′-ethylbiphenyl-2-yl)-1-hydroxy-5- |
| | methoxypentyl)morpholino)methanone |
| I-269 | (4-(aminomethyl)phenyl)(2-(1-(6-chloro-3′-ethylbiphenyl-2-yl)-1-hydroxy-5- |
| | methoxypentyl)morpholino)methanone |
| I-270 | (3-aminocyclopentyl)(2-(1-(3-chloro-2-(naphthalen-2-yl)phenyl)-1-hydroxy-5- |
| | methoxypentyl)morpholino)methanone |
| I-271 | (4-(aminomethyl)phenyl)(3-(1-(3-chloro-2-(o-tolyloxy)phenyl)-1-hydroxy-5- |
| | methoxypentyl)piperidin-1-yl)methanone |
| I-272 | (4-(aminomethyl)phenyl)(3-(1-(2-(2-chloro-6-methylphenoxy)phenyl)-1-hydroxy-5- |
| | methoxypentyl)piperidin-1-yl)methanone |
| I-273 | (3-aminocyclopentyl)(2-(1-(3-chloro-2-(quinolin-3-yl)phenyl)-1-hydroxy-5- |
| | methoxypentyl)morpholino)methanone |
| I-274 | (6-(aminomethyl)pyridin-3-yl)(2-(1-(6-chloro-3′-ethylbiphenyl-2-yl)-1-hydroxy-5- |
| | methoxypentyl)morpholino)methanone |
| I-275 | methyl 4-hydroxy-4-(1-(3-(methylamino)cyclopentyl carbonyl)-3-piperidinyl)-4-{2-[(2- |
| | methylphenyl)oxy]phenyl}butyl)carbamate |
| I-276 | (4-(aminomethyl)phenyl)(2-(1-(6-chloro-2′-fluoro-5′-methylbiphenyl-2-yl)-1-hydroxy- |
| | 5-methoxypentyl)morpholino)methanone |
| I-277 | methyl 4-(6-fluoro-3′-methoxybiphenyl-2-yl)-4-hydroxy-4-(1-(3- |
| | (methylamino)cyclopentanecarbonyl)piperidin-3-yl)butylcarbamate |
| I-278 | N-(4-(1-(3-amino-4-hydroxycyclopentanecarbonyl)piperidin-3-yl)-4-(6-chloro-3′- |
| | ethylbiphenyl-2-yl)-4-hydroxybutyl)acetamide |
| I-279 | methyl 4-(1-(3-aminocyclopentanecarbonyl)piperidin-3-yl)-4-(6-chloro-3′- |
| | ethylbiphenyl-2-yl)-4-hydroxybutylcarbamate |
| I-280 | methyl 4-(4-(3-aminocyclopentanecarbonyl)morpholin-2-yl)-4-(6-chloro-3′- |
| | ethylbiphenyl-2-yl)-4-hydroxybutylcarbamate |
| I-281 | (3-amino-4-hydroxycyclopentyl)(3-(1-(3-chloro-2-(3-ethylphenoxy)phenyl)-1-hydroxy- |
| | 5-methoxypentyl)piperidin-1-yl)methanone |
| I-282 | methyl 4-hydroxy-4-(1-(4-((methylamino)methyl)benzoyl)piperidin-3-yl)-4-(2-(o- |
| | tolyloxy)phenyl)butylcarbamate |
| I-283 | (3-amino-4-hydroxycyclopentyl)(2-(1-(6-fluoro-3′,5′-dimethoxybiphenyl-2-yl)-1- |
| | hydroxy-5-methoxypentyl)morpholino)methanone |
| I-284 | (3-amino-4-hydroxycyclopentyl)(2-(1-(6-chloro-3′-(methoxymethyl)biphenyl-2-yl)-1- |
| | hydroxy-5-methoxypentyl)morpholino)methanone |
| I-285 | (4-(aminomethyl)cyclohexyl)(2-(1-(6-chloro-2′-fluoro-5′-methylbiphenyl-2-yl)-1- |
| | hydroxy-5-methoxypentyl)morpholino)methanone |
| I-286 | N-(4-(6-chloro-3′-ethylbiphenyl-2-yl)-4-hydroxy-4-(1-(4- |
| | ((methylamino)methyl)benzoyl)piperidin-3-yl)butyl)formamide |
| I-287 | (4-(2-aminoethyl)phenyl)((R)-3-((S)-1-(6-chloro-3′-ethylbiphenyl-2-yl)-1-hydroxy-5- |
| | methoxypentyl)piperidin-1-yl)methanone |
| I-288 | (3-(1-(3-chloro-2-(2-methylbenzyl)phenyl)-1-hydroxy-5-methoxypentyl)piperidin-1- |
| | yl)(4-((methylamino)methyl)phenyl)methanone |
| I-289 | methyl 4-(1-(4-(aminomethyl)benzoyl)piperidin-3-yl)-4-(6-fluoro-3′-methoxybiphenyl- |
| | 2-yl)-4-hydroxybutylcarbamate |
| I-290 | (4-(aminomethyl)phenyl)(3-(1-(3-chloro-2-(2-ethylphenoxy)phenyl)-1-hydroxy-5- |
| | methoxypentyl)piperidin-1-yl)methanone |
| I-291 | (4-(aminomethyl)phenyl)(3-(1-(3-chloro-2-(3-ethylphenoxy)phenyl)-1-hydroxy-5- |
| | methoxypentyl)piperidin-1-yl)methanone |
| I-292 | (3-amino-4-hydroxycyclopentyl)(3-(1-(3-chloro-2-(quinolin-3-yl)phenyl)-1-hydroxy-5- |
| | methoxypentyl)piperidin-1-yl)methanone |
| I-292 | (3-amino-4-hydroxycyclopentyl)(3-(1-(3-chloro-2-(quinolin-3-yl)phenyl)-1-hydroxy-5- |
| | methoxypentyl)piperidin-1-yl)methanone |
| I-293 | (4-(aminomethyl)phenyl)(2-(1-(6-chloro-3′-(methoxymethyl)biphenyl-2-yl)-1-hydroxy- |
| | 5-methoxypentyl)morpholino)methanone |
| I-294 | (3-amino-4-hydroxycyclopentyl)(2-(1-(3-chloro-2-(naphthalen-2-yl)phenyl)-1-hydroxy- |
| | 5-methoxypentyl)morpholino)methanone |
| I-295 | (3-amino-4-hydroxycyclopentyl)(2-(1-(3-chloro-2-(quinolin-3-yl)phenyl)-1-hydroxy-5- |
| | methoxypentyl)morpholino)methanone |
| I-296 | (3-amino-4-hydroxycyclopentyl)(2-(1-(3-chloro-2-(isoquinolin-4-yl)phenyl)-1-hydroxy- |
| | 5-methoxypentyl)morpholino)methanone |
| I-296 | (3-amino-4-hydroxycyclopentyl)(2-(1-(3-chloro-2-(isoquinolin-4-yl)phenyl)-1-hydroxy- |
| | 5-methoxypentyl)morpholino)methanone |
| I-297 | methyl 4-(6-chloro-3′-ethylbiphenyl-2-yl)-4-hydroxy-4-(1-(3- |
| | (methylamino)cyclopentanecarbonyl)piperidin-3-yl)butylcarbamate |
| I-298 | methyl 4-(1-(3-aminocyclopentanecarbonyl)piperidin-3-yl)-4-(6-chloro-3′- |
| | isopropylbiphenyl-2-yl)-4-hydroxybutylcarbamate |
| I-299 | methyl 4-(1-(3-amino-4-hydroxycyclopentanecarbonyl)piperidin-3-yl)-4-(6-chloro-3′- |
| | ethylbiphenyl-2-yl)-4-hydroxybutylcarbamate |
| I-300 | methyl 4-(6-chloro-3′-methoxybiphenyl-2-yl)-4-hydroxy-4-(1-(3- |
| | (methylamino)cyclopentanecarbonyl)piperidin-3-yl)butylcarbamate |
| I-301 | (4-(aminomethyl)phenyl)(3-(1-(3-chloro-2-(quinolin-3-yl)phenyl)-1-hydroxy-5- |
| | methoxypentyl)piperidin-1-yl)methanone |
| I-302 | (4-(aminomethyl)phenyl)(2-(1-(3-chloro-2-(naphthalen-2-yl)phenyl)-1-hydroxy-5- |
| | methoxypentyl)morpholino)methanone |
| I-303 | (4-(aminomethyl)phenyl)(2-(1-(3-chloro-2-(quinolin-3-yl)phenyl)-1-hydroxy-5- |
| | methoxypentyl)morpholino)methanone |
| I-303 | (4-(aminomethyl)phenyl)(2-(1-(3-chloro-2-(quinolin-3-yl)phenyl)-1-hydroxy-5- |
| | methoxypentyl)morpholino)methanone |
| I-304 | N-(4-(6-chloro-3′-ethylbiphenyl-2-yl)-4-hydroxy-4-(1-(4- |
| | ((methylamino)methyl)benzoyl)piperidin-3-yl)butyl)acetamide |
| I-305 | (3-amino-4-hydroxycyclopentyl)(2-(1-(6-chloro-3′,5′-dimethoxybiphenyl-2-yl)-1- |
| | hydroxy-5-methoxypentyl)morpholino)methanone |
| I-306 | methyl 4-(6-fluoro-3′-methoxybiphenyl-2-yl)-4-hydroxy-4-(1-(4- |
| | ((methylamino)methyl)benzoyl)piperidin-3-yl)butylcarbamate |
| I-307 | methyl 4-(1-(4-(aminomethyl)benzoyl)piperidin-3-yl)-4-(6-chloro-3′-ethylbiphenyl-2- |
| | yl)-4-hydroxybutylcarbamate |
| I-307 | methyl 4-(1-(4-(aminomethyl)benzoyl)piperidin-3-yl)-4-(6-chloro-3′-ethylbiphenyl-2- |
| | yl)-4-hydroxybutylcarbamate |
| I-308 | methyl 4-(6-chloro-3′-methylbiphenyl-2-yl)-4-hydroxy-4-(1-(4- |
| | ((methylamino)methyl)benzoyl)piperidin-3-yl)butylcarbamate |
| I-309 | methyl 4-(1-(6-(aminomethyl)nicotinoyl)piperidin-3-yl)-4-(6-chloro-3′-ethylbiphenyl-2- |
| | yl)-4-hydroxybutylcarbamate |
| I-310 | methyl 4-(4-(4-(aminomethyl)benzoyl)morpholin-2-yl)-4-(6-chloro-3′-ethylbiphenyl-2- |
| | yl)-4-hydroxybutylcarbamate |
| I-311 | methyl 4-(1-(4-(aminomethyl)benzoyl)piperidin-3-yl)-4-(6-chloro-3′-methoxybiphenyl- |
| | 2-yl)-4-hydroxybutylcarbamate |
| I-312 | methyl 4-(1-(4-(aminomethyl)cyclohexanecarbonyl)piperidin-3-yl)-4-(6-chloro-3′- |
| | ethylbiphenyl-2-yl)-4-hydroxybutylcarbamate |
| I-313 | methyl 4-(1-(3-amino-4-hydroxycyclopentanecarbonyl)piperidin-3-yl)-4-(6-chloro-3′- |
| | isopropylbiphenyl-2-yl)-4-hydroxybutylcarbamate |
| I-314 | N-(4-(6-chloro-3′-ethylbiphenyl-2-yl)-4-hydroxy-4-(1-(4- |
| | ((methylamino)methyl)benzoyl)piperidin-3-yl)butyl)propionamide |
| I-315 | ethyl 4-(1-(4-(aminomethyl)benzoyl)piperidin-3-yl)-4-(6-chloro-3′-ethylbiphenyl-2-yl)- |
| | 4-hydroxybutylcarbamate |
| I-316 | methyl 4-(6-chloro-3′-ethylbiphenyl-2-yl)-4-hydroxy-4-(1-(4- |
| | ((methylamino)methyl)benzoyl)piperidin-3-yl)butylcarbamate |
| I-317 | methyl 4-(1-(4-(aminomethyl)benzoyl)piperidin-3-yl)-4-(6-chloro-3′-isopropylbiphenyl- |
| | 2-yl)-4-hydroxybutylcarbamate |
| I-318 | N-(4-(6-chloro-3′-ethylbiphenyl-2-yl)-4-hydroxy-4-(1-(4- |
| | ((methylamino)methyl)benzoyl)piperidin-3-yl)butyl)-2-hydroxyacetamide |
| I-319 | methyl 4-(6-chloro-3′-methoxybiphenyl-2-yl)-4-hydroxy-4-(1-(4- |
| | ((methylamino)methyl)benzoyl)piperidin-3-yl)butylcarbamate |
| I-320 | (3-aminocyclopentyl)(3-(1-(3′-ethoxy-6-fluoro-5′-(trifluoromethyl)biphenyl-2-yl)-1- |
| | hydroxy-5-methoxypentyl)piperidin-1-yl)methanone |
| I-321 | methyl 4-(1-(3-amino-4-hydroxycyclopentanecarbonyl)piperidin-3-yl)-4-(3-chloro-2- |
| | (quinolin-3-yl)phenyl)-4-hydroxybutylcarbamate |
| I-322 | methyl 4-(1-(4-(aminomethyl)-2-fluorobenzoyl)piperidin-3-yl)-4-(6-chloro-3′- |
| | ethylbiphenyl-2-yl)-4-hydroxybutylcarbamate |
| I-323 | (3-aminocyclopentyl)(2-(1-(3′-ethoxy-6-fluoro-5′-(trifluoromethyl)biphenyl-2-yl)-1- |
| | hydroxy-5-methoxypentyl)morpholino)methanone |
| I-324 | isopropyl 4-(1-(4-(aminomethyl)benzoyl)piperidin-3-yl)-4-(6-chloro-3′-ethylbiphenyl-2- |
| | yl)-4-hydroxybutylcarbamate |
| I-325 | methyl 4-(6-chloro-3′-ethylbiphenyl-2-yl)-4-(1-(4- |
| | ((ethylamino)methyl)benzoyl)piperidin-3-yl)-4-hydroxybutylcarbamate |
| I-326 | ((1S,3R,4S)-3-amino-4-hydroxycyclopentyl)((R)-3-((S)-1-(3′-ethoxy-6-fluoro-5′- |
| | (trifluoromethyl)biphenyl-2-yl)-1-hydroxy-5-methoxypentyl)piperidin-1-yl)methanone |
| I-326 | (3-amino-4-hydroxycyclopentyl)(3-(1-(3′-ethoxy-6-fluoro-5′-(trifluoromethyl)biphenyl- |
| | 2-yl)-1-hydroxy-5-methoxypentyl)piperidin-1-yl)methanone |
| I-327 | (3-amino-4-hydroxycyclopentyl)(2-(1-(3′-ethoxy-6-fluoro-5′-(trifluoromethyl)biphenyl- |
| | 2-yl)-1-hydroxy-5-methoxypentyl)morpholino)methanone |
| I-328 | methyl 4-(6-chloro-3′-ethylbiphenyl-2-yl)-4-hydroxy-4-(1-(4- |
| | ((isopropylamino)methyl)benzoyl)piperidin-3-yl)butylcarbamate |
| I-329 | N-(4-(6-chloro-3′-ethylbiphenyl-2-yl)-4-hydroxy-4-(1-(4- |
| | ((methylamino)methyl)benzoyl)piperidin-3-yl)butyl)-2,2,2-trifluoroacetamide |
| I-330 | (3-aminocyclopentyl)(2-(1-(6-fluoro-3′,5′-dimethoxybiphenyl-2-yl)-1-hydroxy-5- |
| | methoxypentyl)morpholino)methanone |
| I-331 | (3-aminocyclopentyl)(2-(1-(3-chloro-2-(isoquinolin-4-yl)phenyl)-1-hydroxy-5- |
| | methoxypentyl)morpholino)methanone |
| I-332 | methyl (4-((3R)-1-{[(1R,3S)-3-aminocyclopentyl]carbonyl}-3-piperidinyl)-4-{3-chloro- |
| | 2-[(2-methylphenyl)oxy]phenyl}-4-hydroxybutyl)carbamate |
| I-333 | methyl (4-((3R)-1-{[(1R,3S)-3-aminocyclopentyl]carbonyl}-3-piperidinyl)-4-{3-chloro- |
| | 2-[(2-ethylphenyl)oxy]phenyl}-4-hydroxybutyl)carbamate |
| I-334 | methyl (4-((3R)-1-{[(1S,3R,4S)-3-amino-4-hydroxycyclopentyl]carbonyl}-3- |
| | piperidinyl)-4-{3-chloro-2-[(2-methylphenyl)oxy]phenyl}-4-hydroxybutyl)carbamate |
| I-335 | methyl (4-((3R)-1-{[(1S,3R,4S)-3-amino-4-hydroxycyclopentyl]carbonyl}-3- |
| | piperidinyl)-4-{3-chloro-2-[(2-ethylphenyl)oxy]phenyl}-4-hydroxybutyl)carbamate |
| I-336 | methyl [4-((3R)-1-{[(1S,3R,4S)-3-amino-4-hydroxycyclopentyl]carbonyl}-3- |
| | piperidinyl)-4-(3′-ethyl-6-fluoro-2-biphenylyl)-4-hydroxybutyl]carbamate |
| I-337 | methyl {4-(6-chloro-3′-ethyl-2-biphenylyl)-4-hydroxy-4-[(3R)-1-({trans-4- |
| | [(methylamino)methyl]cyclohexyl}carbonyl)-3-piperidinyl]butyl}carbamate |
| I-338 | methyl {4-(3′-ethyl-6-fluoro-2-biphenylyl)-4-hydroxy-4-[(3R)-1-({4- |
| | [(methylamino)methyl]phenyl}carbonyl)-3-piperidinyl]butyl}carbamate |
| I-339 | methyl {4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}carbamate |
| I-340 | N-{4-(2′,6-difluoro-5′-methyl-2-biphenylyl)-4-hydroxy-4-[(3R)-1-({4- |
| | [(methylamino)methyl]phenyl}carbonyl)-3-piperidinyl]butyl}-2-hydroxyacetamide |

or a diastereomer, enantiomer or salt thereof.

The following are compounds of the invention:

| Cpd. | | |
|---|---|---|
| No. | Structure | Name |
| I-1a | 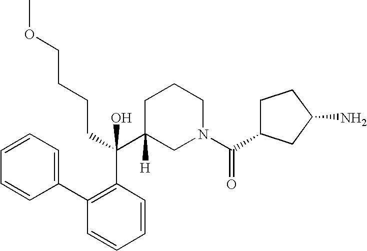 | ((1R,3S)-3- aminocyclopentyl)((R)-3-(1- (2-phenylphenyl)-1-hydroxy- 5-methoxypentyl)piperidin-1- yl)methanone |
| I-2a | 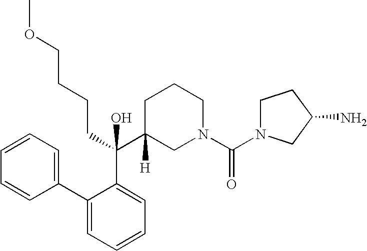 | ((S)-3-aminopyrrolidin-1- yl)((R)-3-(1-(2- phenylphenyl)-1-hydroxy-5- methoxypentyl)piperidin-1- yl)methanone |
| I-3a | 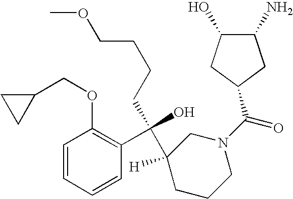 | ((1S,3R,4S)-3-amino-4- hydroxycyclopentyl)((R)-3- (1-(2- (cyclopropylmethoxy)phenyl)- 1-hydroxy-5- methoxypentyl)piperidin-1- yl)methanone |
| I-3b | 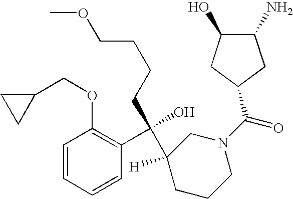 | ((1S,3R,4R)-3-amino-4- hydroxycyclopentyl)((R)-3- (1-(2- (cyclopropylmethoxy)phenyl)- 1-hydroxy-5- methoxypentyl)piperidin-1- yl)methanone |
| I-4a | 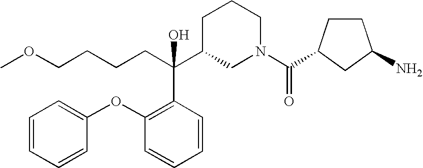 | ((1R,3S)-3- aminocyclopentyl)((R)-3- ((S)-1-hydroxy-5-methoxy-1- (2-phenoxyphenyl) pentyl)piperidin- 1-yl)methanone |
| I-4b | 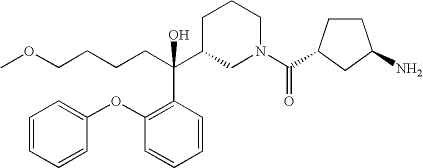 | ((1R,3R)-3- aminocyclopentyl)((R)-3- ((S)-1-hydroxy-5-methoxy-1- (2-phenoxyphenyl) pentyl)piperidin- 1-yl)methanone |
| I-5a | 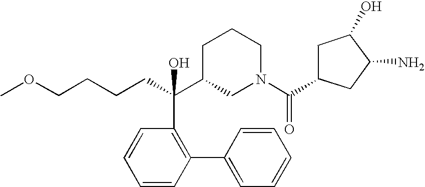 | ((1S,3R,4S)-3-amino-4- hydroxycyclopentyl)((R)-3- ((S)-1-(2-phenylphenyl)-1- hydroxy-5- methoxypentyl)piperidin-1- yl)methanone |
| I-6a | 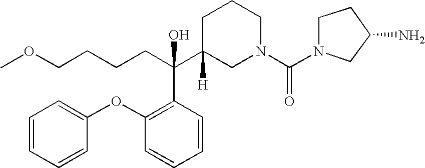 | ((S)-3-aminopyrrolidin-1- yl)((R)-3-((S)-1-hydroxy-5- methoxy-1-(2- phenoxyphenyl)pentyl) piperidin-1-yl)methanone |
| I-7a | 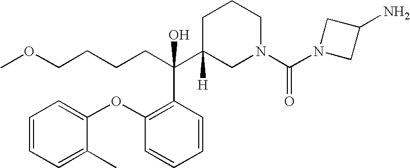 | ((R)-3-((S)-1-(2-(o- tolyloxy)phenyl)-1-hydroxy- 5-methoxypentyl)piperidin-1- yl)(3-aminoazetidin-1- yl)methanone |
| I-8a | 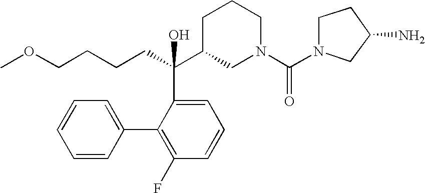 | ((S)-3-aminopyrrolidin-1- yl)((R)-3-((S)-1-(2-ethyl-3- fluorophenyl)-1-hydroxy-5- methoxypentyl)piperidin-1- yl)methanone |
| I-9a | 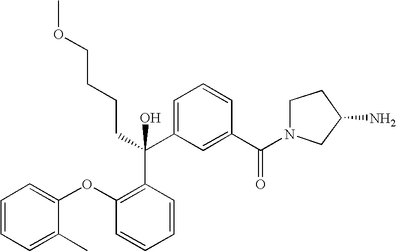 | ((S)-3-aminopyrrolidin-1- yl)(3-((S)-1-hydroxy-5- methoxy-1-(2-(o- tolyloxy)phenyl)pentyl) phenyl)methanone |
| I-10a | 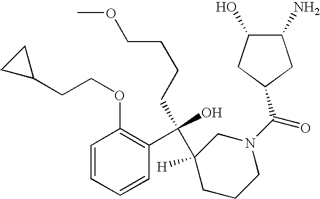 | ((1S,3R,4S)-3-amino-4- hydroxycyclopentyl)((R)-3- ((S)-1-(2-(2- cyclopropylethoxy)phenyl)- 1-hydroxy-5- methoxypentyl)piperidin-1- yl)methanone |
| I-10b | 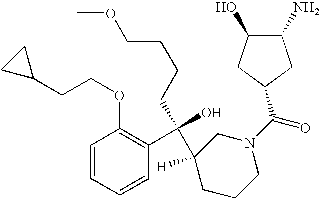 | ((1S,3R,4R)-3-amino-4- hydroxycyclopentyl)((R)-3- ((S)-1-(2-(2- cyclopropylethoxy)phenyl)- 1-hydroxy-5- methoxypentyl)piperidin-1- yl)methanone |
| I-11a | 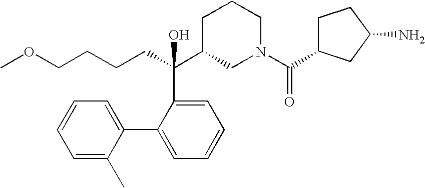 | ((1R,3S)-3- aminocyclopentyl)((3R)-3- ((1S)-1-hydroxy-5-methoxy- 1-(2′-methylbiphenyl-2- yl)pentyl)piperidin-1- yl)methanone |
| I-12a | 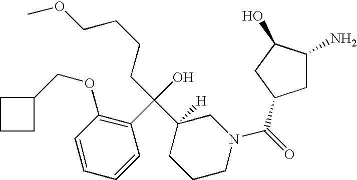 | ((1S,3R,4R)-3-amino-4- hydroxycyclopentyl)(3-(1-(2- (cyclobutylmethoxy)phenyl)- 1-hydroxy-5- methoxypentyl)piperidin-1- yl)methanone |
| I-12b | 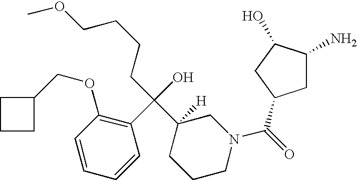 | ((1S,3R,4S)-3-amino-4- hydroxycyclopentyl)(3-(1-(2- (cyclobutylmethoxy)phenyl)- 1-hydroxy-5- methoxypentyl)piperidin-1- yl)methanone |
| I-13a | 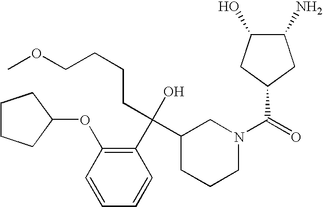 | ((1S,3R,4S)-3-amino-4- hydroxycyclopentyl)(3-(1-(2- (cyclopentyloxy)phenyl)-1- hydroxy-5- methoxypentyl)piperidin-1- yl)methanone |
| I-14a | 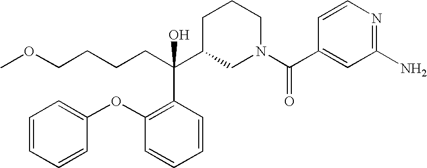 | (2-aminopyridin-4-yl)((R)-3- ((S)-1-hydroxy-5-methoxy-1- (2-phenoxyphenyl)pentyl) piperidin-1-yl)methanone |
| I-15a | 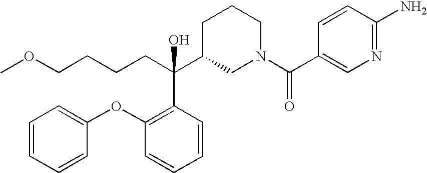 | (6-aminopyridin-3-yl)((R)-3- ((S)-1-hydroxy-5-methoxy-1- (2-phenoxyphenyl)pentyl) piperidin-1-yl)methanone |
| I-16a | 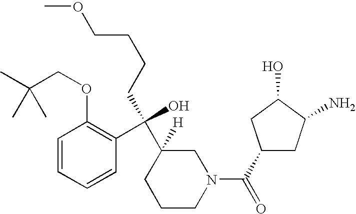 | ((1S,3R,4S)-3-amino-4- hydroxycyclopentyl)((R)-3- ((S)-1-hydroxy-5-methoxy-1- (2-(neopentyloxy)phenyl)pentyl) piperidin-1-yl)methanone |
| I-17a | 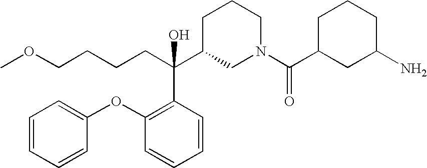 | (3-aminocyclohexyl)((R)-3- ((S)-1-hydroxy-5-methoxy-1- (2-phenoxyphenyl)pentyl) piperidin-1-yl)methanone |
| I-18a | 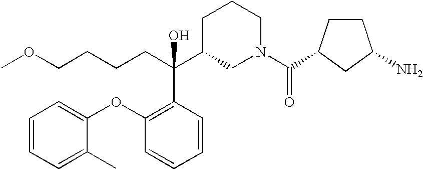 | ((R)-3-((S)-1-(2-(o- tolyloxy)phenyl)-1-hydroxy- 5-methoxypentyl)piperidin-1- yl)((1R,3S)-3- aminocyclopentyl)methanone |
| I-19a | 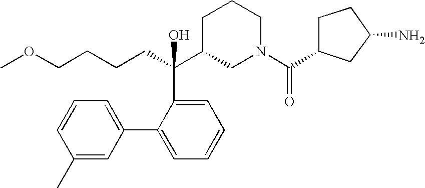 | ((1R,3S)-3- aminocyclopentyl)((R)-3- ((S)-1-hydroxy-5-methoxy-1- (3′-methylbiphenyl-2- yl)pentyl)piperidin-1- yl)methanone |
| I-20a | 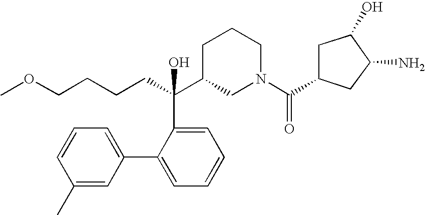 | ((1S,3R,4S)-3-amino-4- hydroxycyclopentyl)((R)-3- ((S)-1-(2-phenylphenyl)-1- hydroxy-5- methoxypentyl)piperidin-1- yl)methanone |
| I-21a | 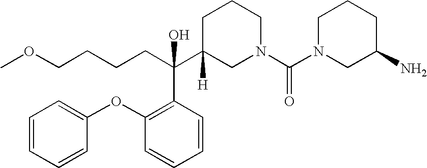 | ((R)-3-aminopiperidin-1- yl)((R)-3-((S)-1-hydroxy-5- methoxy-1-(2- phenoxyphenyl)pentyl) piperidin-1-yl)methanone |
| I-22a | 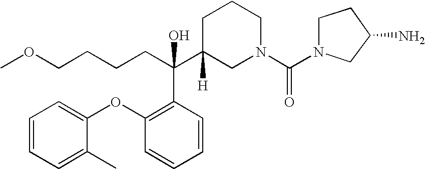 | ((R)-3-((S)-1-(2-(o- tolyloxy)phenyl)-1-hydroxy- 5-methoxypentyl)piperidin-1- yl)((S)-3-aminopyrrolidin-1- yl)methanone |
| I-22b | 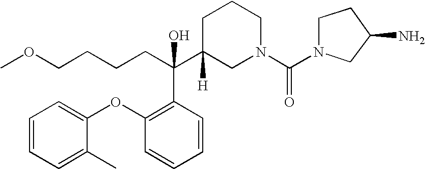 | ((R)-3-((S)-1-(2-(o- tolyloxy)phenyl)-1-hydroxy- 5-methoxypentyl)piperidin-1- yl)((R)-3-aminopyrrolidin-1- yl)methanone |
| I-23a | 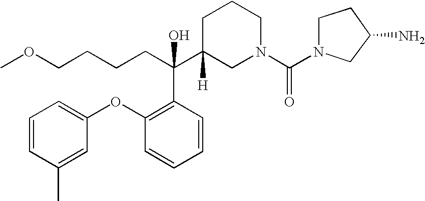 | ((R)-3-((S)-1-(2-(m- tolyloxy)phenyl)-1-hydroxy- 5-methoxypentyl)piperidin-1- yl)((S)-3-aminopyrrolidin-1- yl)methanone |
| I-24a | 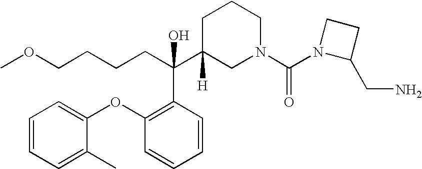 | ((R)-3-((S)-1-(2-(o- tolyloxy)phenyl)-1-hydroxy- 5-methoxypentyl)piperidin-1- yl)(2-(aminomethyl)azetidin- 1-yl)methanone |
| I-25a | 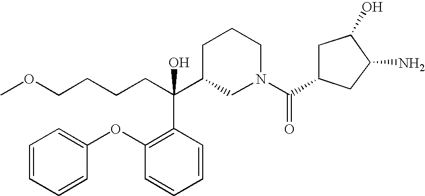 | ((1S,3R,4S)-3-amino-4- hydroxycyclopentyl)((R)-3- ((S)-1-hydroxy-5-methoxy-1- (2-phenoxyphenyl)pentyl) piperidin-1-yl)methanone |
| I-25b | 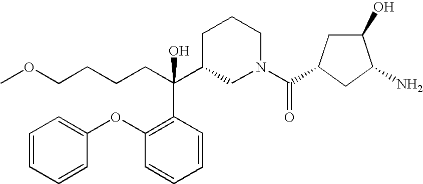 | ((1S,3R,4R)-3-amino-4- hydroxycyclopentyl)((R)-3- ((S)-1-hydroxy-5-methoxy-1- (2-phenoxyphenyl)pentyl) piperidin-1-yl)methanone |
| I-26a | 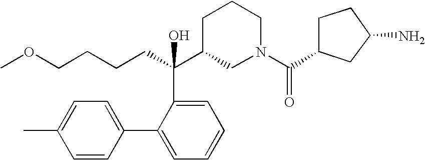 | ((1R,3S)-3- aminocyclopentyl)((R)-3- ((S)-1-hydroxy-5-methoxy-1- (4′-methylbiphenyl-2- yl)pentyl)piperidin-1- yl)methanone |
| I-27a | 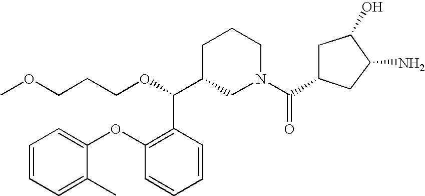 | ((R)-3-((R)-(3- methoxypropoxy)(2-(o- tolyloxy)phenyl)methyl) piperidin-1-yl)((1S,3R,4S)-3- amino-4-hydroxy- cyclopentyl)methanone |
| I-27b | 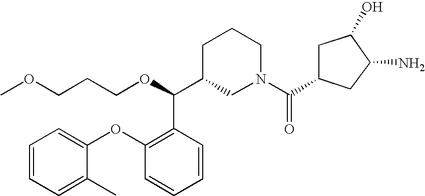 | ((R)-3-((S)-(3- methoxypropoxy)(2-(o- tolyloxy)phenyl)methyl) piperidin-1-yl)((1S,3R,4S)-3- amino-4-hydroxy- cyclopentyl)methanone |
| I-28a | 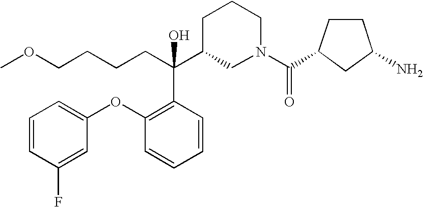 | ((R)-3-((S)-1-(2-(3- fluorophenoxy)phenyl)-1- hydroxy-5- methoxypentyl)piperidin-1- yl)((1R,3S)-3- aminocyclopentyl)methanone |
| I-29a | 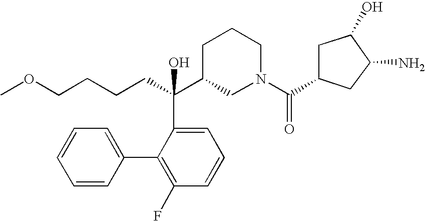 | ((1S,3R,4S)-3-amino-4- hydroxycyclopentyl)((R)-3- ((S)-1-(2-phenyl-3- fluorophenyl)-1-hydroxy-5- methoxypentyl)piperidin-1- yl)methanone |
| I-30a | 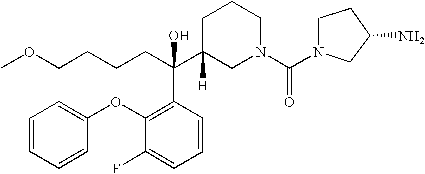 | ((S)-3-aminopyrrolidin-1- yl)((R)-3-((S)-1-(3-fluoro-2- phenoxyphenyl)-1-hydroxy- 5-methoxypentyl)piperidin-1- yl)methanone |
| I-31a | 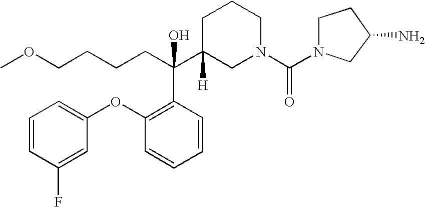 | ((R)-3-((S)-1-(2-(3- fluorophenoxy)phenyl)-1- hydroxy-5- methoxypentyl)piperidin-1- yl)((S)-3-aminopyrrolidin-1- yl)methanone |
| I-32a | 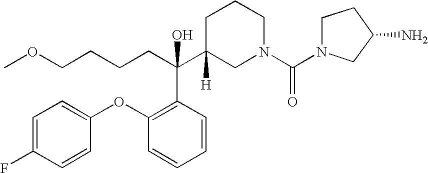 | ((R)-3-((S)-1-(2-(4- fluorophenoxy)phenyl)-1- hydroxy-5- methoxypentyl)piperidin-1- yl)((S)-3-aminopyrrolidin-1- yl)methanone |
| I-33a | 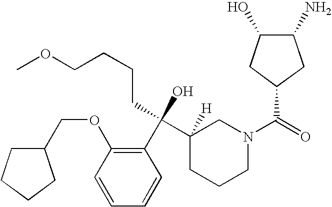 | ((1S,3R,4S)-3-amino-4- hydroxycyclopentyl)((R)-3- ((S)-1-(2- (cyclopentylmethoxy)phenyl)- 1-hydroxy-5- methoxypentyl)piperidin-1- yl)methanone |
| I-34a | 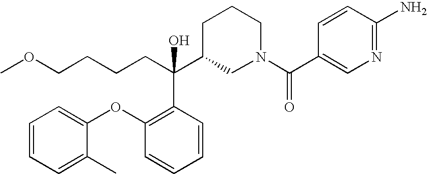 | ((R)-3-((S)-1-(2-(o- tolyloxy)phenyl)-1-hydroxy- 5-methoxypentyl)piperidin-1- yl)(6-aminopyridin-3- yl)methanone |
| I-35a | 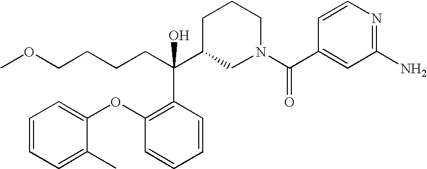 | ((R)-3-((S)-1-(2-(o- tolyloxy)phenyl)-1-hydroxy- 5-methoxypentyl)piperidin-1- yl)(2-aminopyridin-4- yl)methanone |
| 1-36a | 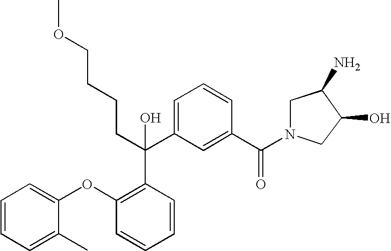 | (3-(1-(2-(o-tolyloxy)phenyl)- 1-hydroxy-5- methoxypentyl)phenyl)((3R, 4S)-3-amino-4- hydroxypyrrolidin-1- yl)methanone |
| I-37a | 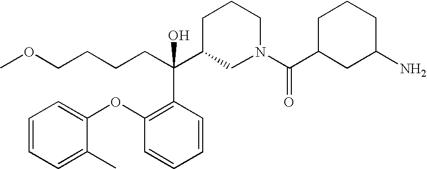 | ((R)-3-((S)-1-(2-(o- tolyloxy)phenyl)-1-hydroxy- 5-methoxypentyl)piperidin-1- yl)(3- aminocyclohexyl)methanone |
| I-37b | 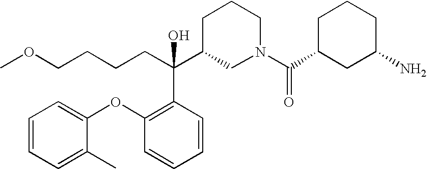 | ((R)-3-((S)-1-(2-(o- tolyloxy)phenyl)-1-hydroxy- 5-methoxypentyl)piperidin-1- yl)((1R,3S)-3- aminocyclohexyl)methanone |
| I-38a | 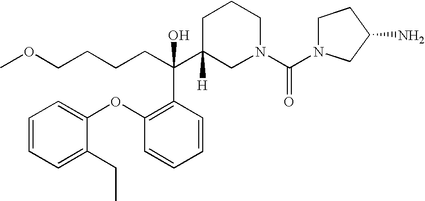 | ((R)-3-((S)-1-(2-(2- ethylphenoxy)phenyl)-1- hydroxy-5- methoxypentyl)piperidin-1- yl)((S)-3-aminopyrrolidin-1- yl)methanone |
| I-39a | 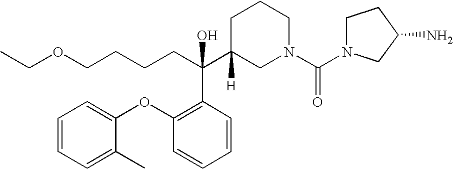 | ((R)-3-((S)-1-(2-(o- tolyloxy)phenyl)-5-ethoxy-1- hydroxypentyl)piperidin-1- yl)((S)-3-aminopyrrolidin-1- yl)methanone |
| I-40a | 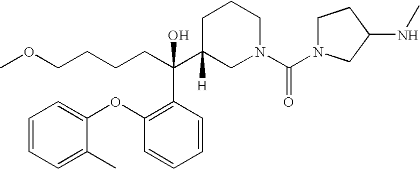 | ((R)-3-((S)-1-(2-(o- tolyloxy)phenyl)-1-hydroxy- 5-methoxypentyl)piperidin-1- yl)(3- (methylamino)pyrrolidin-1- yl)methanone |
| I-41a | 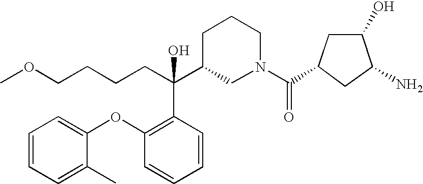 | ((R)-3-((S)-1-(2-(o- tolyloxy)phenyl)-1-hydroxy- 5-methoxypentyl)piperidin-1- yl)((1S,3R,4S)-3-amino-4- hydroxycyclopentyl) methanone |
| I-41b | 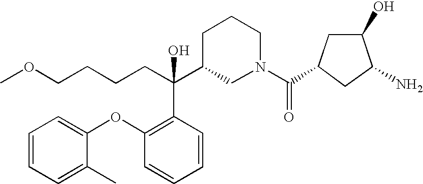 | ((R)-3-((S)-1-(2-(o- tolyloxy)phenyl)-1-hydroxy- 5-methoxypentyl)piperidin-1- yl)((1S,3R,4R)-3-amino-4- hydroxycyclopentyl) methanone |
| I-42a | 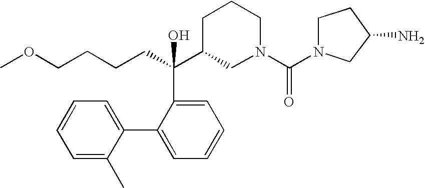 | ((S)-3-aminopyrrolidin-1- yl)((3R)-3-((1S)-1-hydroxy- 5-methoxy-1-(2′- methylbiphenyl-2- yl)pentyl)piperidin-1- yl)methanone |
| I-43a | 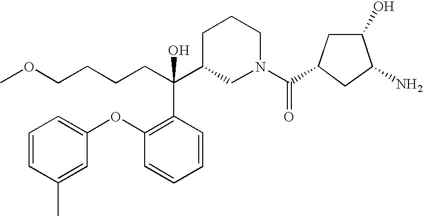 | ((R)-3-((S)-1-(2-(m- tolyloxy)phenyl)-1-hydroxy- 5-methoxypentyl)piperidin-1- yl)((1S,3R,4S)-3-amino-4- hydroxycyclopentyl) methanone |
| I-44a | 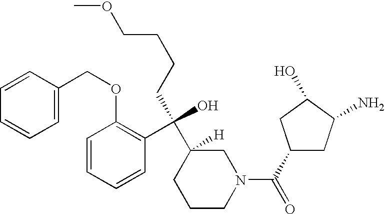 | ((1S,3R,4S)-3-amino-4- hydroxycyclopentyl)((R)-3- ((S)-1-(2- (benzyloxy)phenyl)-1- hydroxy-5- methoxypentyl)piperidin-1- yl)methanone |
| I-45a | 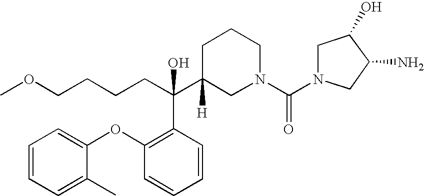 | ((R)-3-((S)-1-(2-(o- tolyloxy)phenyl)-1-hydroxy- 5-methoxypentyl)piperidin-1- yl)((3R,4S)-3-amino-4- hydroxypyrrolidin-1- yl)methanone |
| I-46a | 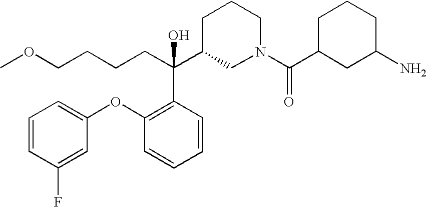 | ((R)-3-((S)-1-(2-(3- fluorophenoxy)phenyl)-1- hydroxy-5- methoxypentyl)piperidin-1- yl)(3- aminocyclohexyl)methanone |
| I-47a | 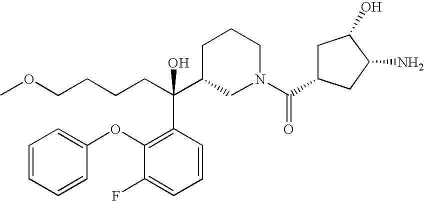 | ((1S,3R,4S)-3-amino-4- hydroxycyclopentyl)((R)-3- ((S)-1-(3-fluoro-2- phenoxyphenyl)-1-hydroxy- 5-methoxypentyl)piperidin-1- yl)methanone |
| I-47b | 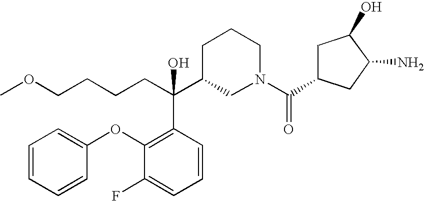 | ((1S,3R,4R)-3-amino-4- hydroxycyclopentyl)((R)-3- ((S)-1-(3-fluoro-2- phenoxyphenyl)-1-hydroxy- 5-methoxypentyl)piperidin-1- yl)methanone |
| I-48a | 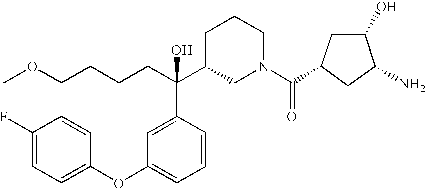 | ((R)-3-((S)-1-(3-(4- fluorophenoxy)phenyl)-1- hydroxy-5- methoxypentyl)piperidin-1- yl)((1S,3R,4S)-3-amino-4- hydroxycyclopentyl) methanone |
| I-49a | 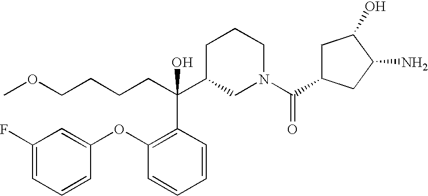 | ((R)-3-((S)-1-(2-(3- fluorophenoxy)phenyl)-1- hydroxy-5- methoxypentyl)piperidin-1- yl)((1S,3R,4S)-3-amino-4- hydroxycyclopentyl) methanone |
| I-50a | 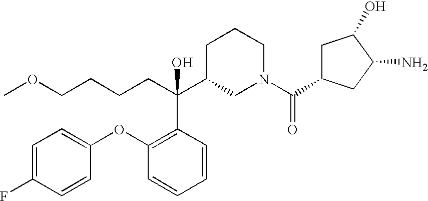 | ((R)-3-((S)-1-(2-(4- fluorophenoxy)phenyl)-1- hydroxy-5- methoxypentyl)piperidin-1- yl)((1S,3R,4S)-3-amino-4- hydroxycyclopentyl) methanone |
| I-51a | 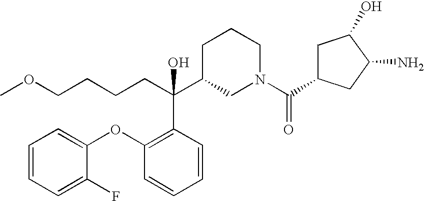 | ((R)-3-((S)-1-(2-(2- fluorophenoxy)phenyl)-1- hydroxy-5- methoxypentyl)piperidin-1- yl)((1S,3R,4S)-3-amino-4- hydroxycyclopentyl) methanone |
| I-52a | 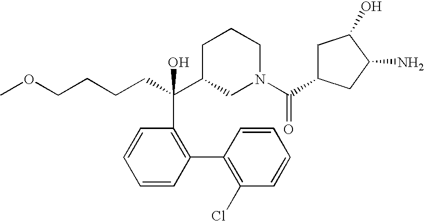 | ((1S,3R,4S)-3-amino-4- hydroxycyclopentyl)((R)-3- ((S)-1-(2-(2- chlorophenyl)phenyl)-1- hydroxy-5- methoxypentyl)piperidin-1- yl)methanone |
| I-53a | 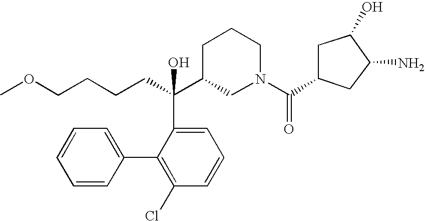 | ((1S,3R,4S)-3-amino-4- hydroxycyclopentyl)((R)-3- ((S)-1-(3-chloro-2- phenylphenyl)-1-hydroxy-5- methoxypentyl)piperidin-1- yl)methanone |
| I-54a | 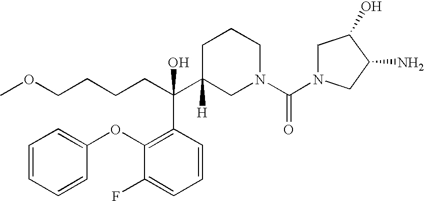 | ((3R,4S)-3-amino-4- hydroxypyrrolidin-1-yl)((R)- 3-((S)-1-(3-fluoro-2- phenoxyphenyl)-1-hydroxy- 5-methoxypentyl)piperidin-1- yl)methanone |
| I-55a | 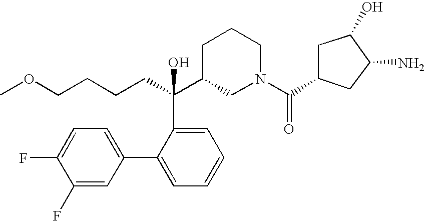 | ((1S,3R,4S)-3-amino-4- hydroxycyclopentyl)((R)-3- ((S)-1-(2-phenylphenyl)-1- hydroxy-5- methoxypentyl)piperidin-1- yl)methanone |
| I-56a | 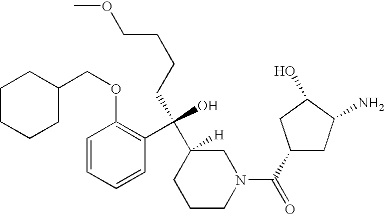 | ((1S,3R,4S)-3-amino-4- hydroxycyclopentyl)((R)-3- ((S)-1-(2- (cyclohexylmethoxy)phenyl)- 1-hydroxy-5- methoxypentyl)piperidin-1- yl)methanone |
| I-57a | 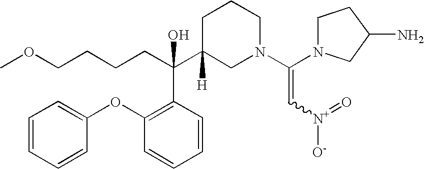 | (S)-1-((R)-1-(1-(3- aminopyrrolidin-1-yl)-2- nitrovinyl)piperidin-3-yl)-5- methoxy-1-(2- phenoxyphenyl)pentan-1-ol |
| I-58a | 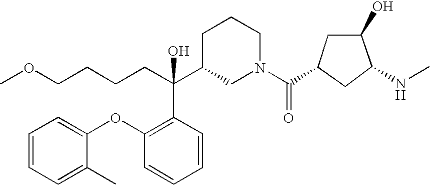 | ((R)-3-((S)-1-(2-(o- tolyloxy)phenyl)-1-hydroxy- 5-methoxypentyl)piperidin-1- yl)((1S,3R,4R)-3-hydroxy-4- (methylamino)cyclopentyl) methanone |
| I-58b | 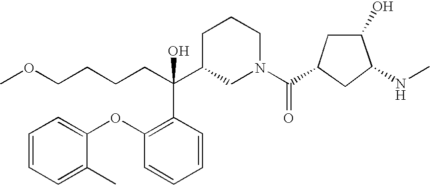 | ((R)-3-((S)-1-(2-(o- tolyloxy)phenyl)-1-hydroxy- 5-methoxypentyl)piperidin-1- yl)((1S,3S,4R)-3-hydroxy-4- (methylamino)cyclopentyl) methanone |
| I-58c | 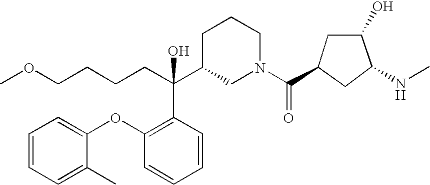 | ((R)-3-((S)-1-(2-(o- tolyloxy)phenyl)-1-hydroxy- 5-methoxypentyl)piperidin-1- yl)((1R,3S,4R)-3-hydroxy-4- (methylamino)cyclopentyl) methanone |
| I-59a | 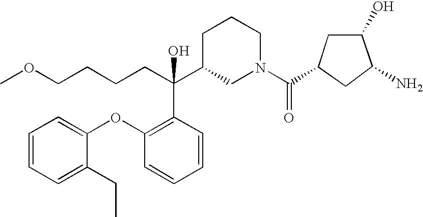 | ((R)-3-((S)-1-(2-(2- ethylphenoxy)phenyl)-1- hydroxy-5- methoxypentyl)piperidin-1- yl)((1S,3R,4S)-3-amino-4- hydroxycyclopentyl) methanone |
| I-60a | 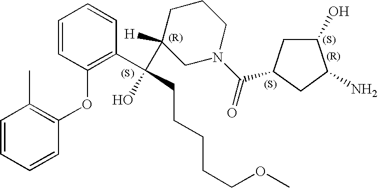 | ((R)-3-((S)-1-(2-(o- tolyloxy)phenyl)-1-hydroxy- 6-methoxyhexyl)piperidin-1- yl)((1S,3R,4S)-3-amino-4- hydroxycyclopentyl) methanone |
| I-61a | 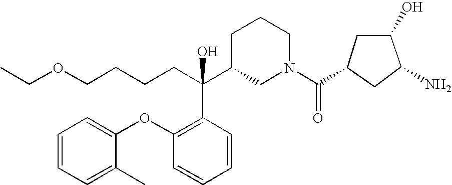 | ((R)-3-((S)-1-(2-(o- tolyloxy)phenyl)-5-ethoxy-1- hydroxypentyl)piperidin-1- yl)((1S,3R,4S)-3-amino-4- hydroxycyclopentyl) methanone |
| I-62a | 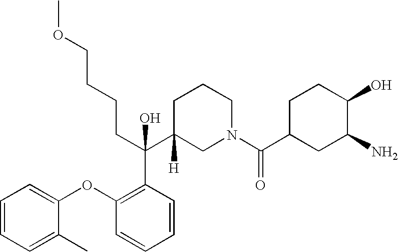 | ((R)-3-((S)-1-(2-(o- tolyloxy)phenyl)-1-hydroxy- 5-methoxypentyl)piperidin-1- yl)((3S,4R)-3-amino-4- hydroxycyclohexyl) methanone |
| I-62b | 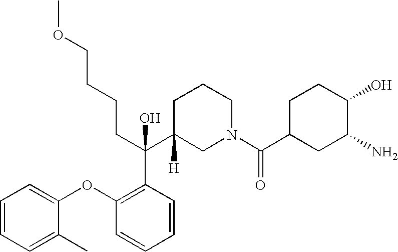 | ((R)-3-((S)-1-(2-(o- tolyloxy)phenyl)-1-hydroxy- 5-methoxypentyl)piperidin-1- yl)((3R,4S)-3-amino-4- hydroxycyclohexyl) methanone |
| I-63a | 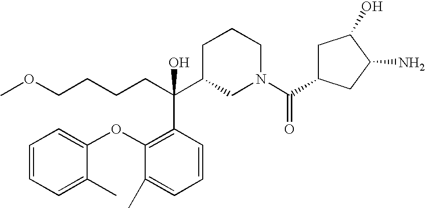 | ((R)-3-((S)-1-(2-(o-tolyloxy)- 3-methylphenyl)-1-hydroxy- 5-methoxypentyl)piperidin-1- yl)((1S,3R,4S)-3-amino-4- hydroxycyclopentyl) methanone |
| I-64a | 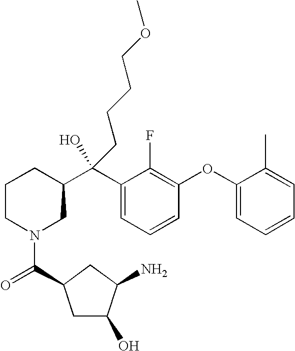 | ((R)-3-((S)-1-(3-(o-tolyloxy)- 2-fluorophenyl)-1-hydroxy- 5-methoxypentyl)piperidin-1- yl)((1S,3R,4S)-3-amino-4- hydroxycyclopentyl) methanone |
| I-65a | 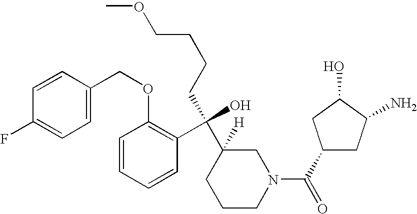 | ((1S,3R,4S)-3-amino-4- hydroxycyclopentyl)((R)-3- ((S)-1-(2-(4- fluorobenzyloxy)phenyl)-1- hydroxy-5- methoxypentyl)piperidin-1- yl)methanone |
| I-66a | 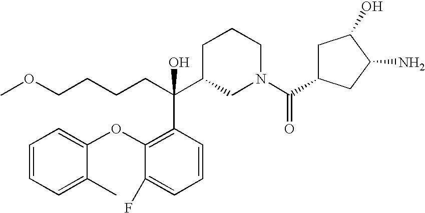 | ((R)-3-((S)-1-(2-(o-tolyloxy)- 3-fluorophenyl)-1-hydroxy- 5-methoxypentyl)piperidin-1- yl)((1S,3R,4S)-3-amino-4- hydroxycyclopentyl) methanone |
| I-67a | 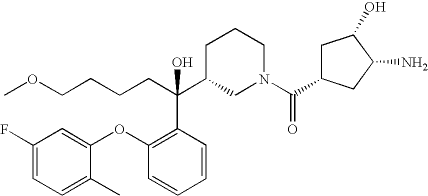 | ((R)-3-((S)-1-(2-(5-fluoro-2- methylphenoxy)phenyl)-1- hydroxy-5- methoxypentyl)piperidin-1- yl)((1S,3R,4S)-3-amino-4- hydroxycyclopenlyl) methanone |
| I-68a | 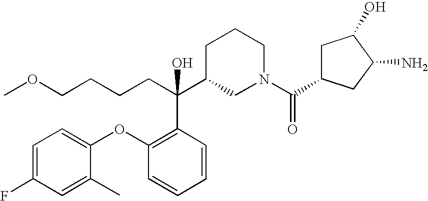 | ((R)-3-((S)-1-(2-(4-fluoro-2- methylphenoxy)phenyl)-1- hydroxy-5- methoxypentyl)piperidin-1- yl)((1S,3R,4S)-3-amino-4- hydroxycyclopenlyl) methanone |
| I-69a | 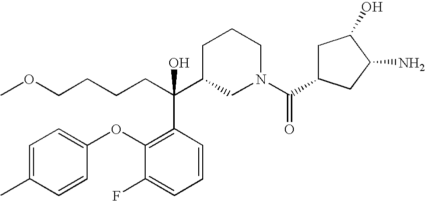 | ((R)-3-((S)-1-(2-(p-tolyloxy)- 3-fluorophenyl)-1-hydroxy- 5-methoxypentyl)piperidin-1- yl)((1S,3R,4S)-3-amino-4- hydroxycyclopentyl) methanone |
| I-70a | 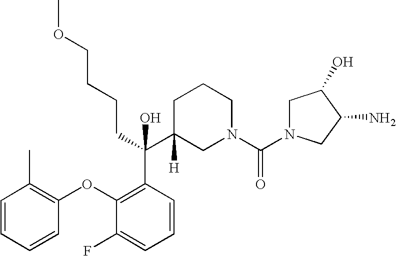 | ((R)-3-((S)-1-(2-(o-tolyloxy)- 3-fluorophenyl)-1-hydroxy- 5-methoxypentyl)piperidin-1- yl)((3R,4S)-3-amino-4- hydroxypyrrolidin-1- yl)methanone |
| I-71a | 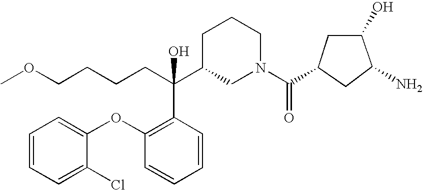 | ((R)-3-((S)-1-(2-(2- chlorophenoxy)phenyl)-1- hydroxy-5- methoxypentyl)piperidin-1- yl)((1S,3R,4S)-3-amino-4- hydroxycyclopentyl) methanone |
| I-72a | 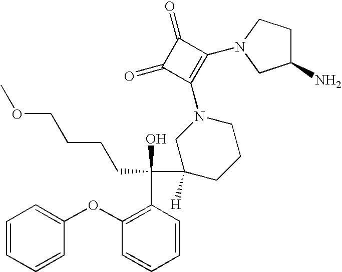 | 3-((R)-3-aminopyrrolidin-1- yl)-4-((R)-3-((S)-1-hydroxy- 5-methoxy-1-(2- phenoxyphenyl)pentyl) piperidin-1-yl)cyclobut- 3-ene-1,2-dione |
| I-73a | 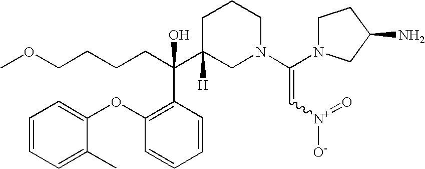 | (S)-1-(2-(o-tolyloxy)phenyl)- 1-((R)-1-(1-((R)-3- aminopyrrolidin-1-yl)-2- nitrovinyl)piperidin-3-yl)-5- methoxypentan-1-ol |
| I-73b | 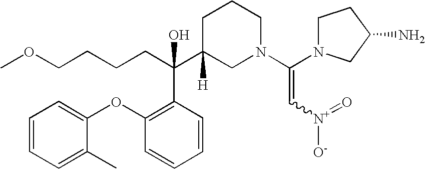 | (S)-1-(2-(o-tolyloxy)phenyl)- 1-((R)-1-(1-((S)-3- aminopyrrolidin-1-yl)-2- nitrovinyl)piperidrn-3-yl)-5- methoxypentan-1-ol |
| I-74a | 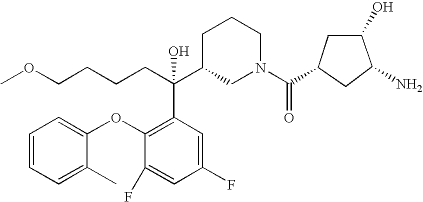 | ((R)-3-((R)-1-(2-(o- tolyloxy)-3,5- difluorophenyl)-1-hydroxy-5- methoxypentyl)piperidin-1- yl)((1S,3R,4S)-3-amino-4- hydroxycyclopentyl) methanone |
| I-74b | 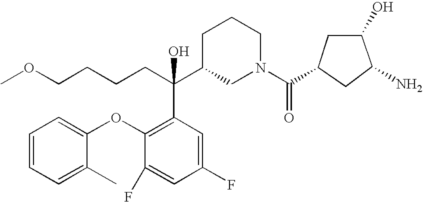 | ((R)-3-((S)-1-(2-(o-tolyloxy)- 3,5-difluorophenyl)-1- hydroxy-5- methoxypentyl)piperidin-1- yl)((1S,3R,4S)-3-amino-4- hydroxycyclopentyl) methanone |
| I-75a | 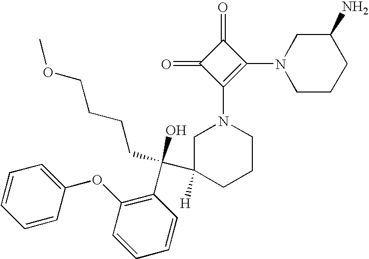 | 3-((S)-3-aminopiperidin-1- yl)-4-((R)-3-((S)-1-hydroxy- 5-methoxy-1-(2- phenoxyphenyl)pentyl) piperidin-1-yl)cyclobut- 3-ene-1,2-dione |
| I-76a | 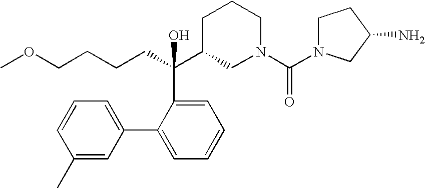 | ((S)-3-aminopyrrolidin-1- yl)((R)-3-((S)-1-hydroxy-5- methoxy-1-(3′- methylbiphenyl-2- yl)pentyl)piperidin-1- yl)methanone |
| I-77a | 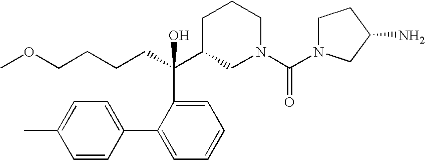 | ((S)-3-aminopyrrolidin-1- yl)((R)-3-((S)-1-hydroxy-5- methoxy-1-(4′- methylbiphenyl-2- yl)pentyl)piperidin-1- yl)methanone |
| I-78a | 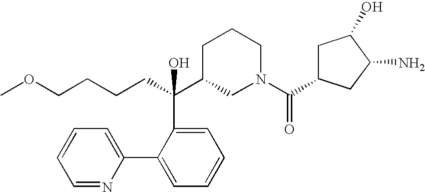 | ((1S,3R,4S)-3-amino-4- hydroxycyclopentyl)((R)-3- ((S)-1-hydroxy-5-methoxy-1- (2-(pyridin-2- yl)phenyl)pentyl)piperidin-1- yl)methanone |
| I-79a | 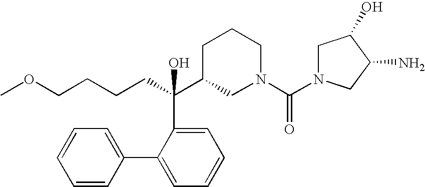 | ((3R,4S)-3-amino-4- hydroxypyrrolidin-1-yl)((R)- 3-((S)-1-(biphenyl-2-yl)-1- hydroxy-5- methoxypentyl)piperidin-1- yl)methanone |
| I-80a | 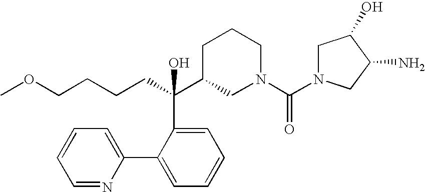 | ((3R,4S)-3-amino-4- hydroxypyrrolidin-1-yl)((R)- 3-((S)-1-hydroxy-5-methoxy- 1-(2-(pyridin-2- yl)phenyl)pentyl)piperidin-1- yl)methanone |
| I-81a | 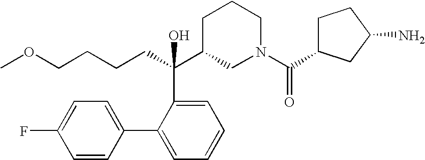 | ((1R,3S)-3- aminocyclopentyl)((R)-3- ((S)-1-(4′-fluorobipbenyl-2- yl)-1-hydroxy-5- methoxypentyl)piperidin-1- yl)methanone |
| I-82a | 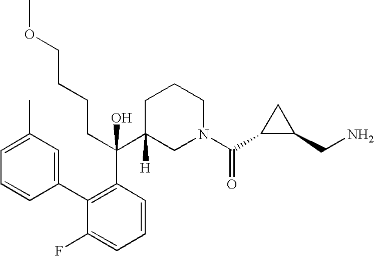 | ((1R,2R)-2- (aminomethyl)cyclopropyl)((R)- 3-((S)-1-(6-fluoro-3′- methylbiphenyl-2-yl)-1- hydroxy-5- methoxypentyl)piperidin-1- yl)methanone |
| I-83a | 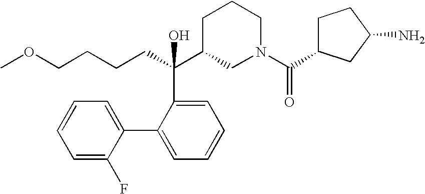 | ((1R,3S)-3- aminocyclopentyl)((3R)-3- ((1S)-1-(2′-fluorobiphenyl-2- yl)-1-hydroxy-5- methoxypentyl)piperidin-1- yl)methanone |
| I-84a | 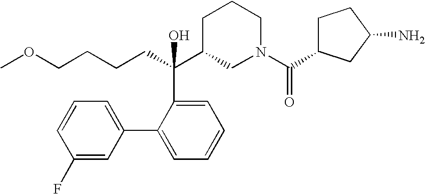 | ((1R,3S)-3- aminocyclopentyl)((R)-3- ((S)-1-(3′-fluorobiphenyl-2- yl)-1-hydroxy-5- methoxypentyl)piperidin-1- yl)methanone |
| I-85a | 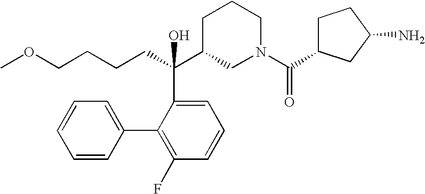 | ((1R,3S)-3- aminocyclopentyl)((R)-3- ((S)-1-(6-fluorobiphenyl-2- yl)-1-hydroxy-5- methoxypentyl)piperidin-1- yl)methanone |
| I-86a | 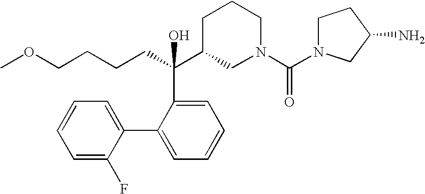 | ((S)-3-aminopyrrolidin-1- yl)((3R)-3-((1S)-1-(2′- fluorobiphenyl-2-yl)-1- hydroxy-5- methoxypentyl)piperidin-1- yl)methanone |
| I-87a | 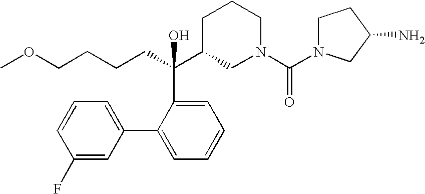 | ((S)-3-aminopyrrolidin-1- yl)((R)-3-((S)-1-(3′- fluorobiphenyl-2-yl)-1- hydroxy-5- methoxypentyl)piperidin-1- yl)methanone |
| I-88a | 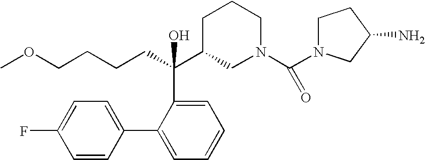 | ((S)-3-aminopyrrolidin-1- yl)((R)-3-((S)-1-(4′- fluorobiphenyl-2-yl)-1- hydroxy-5- methoxypentyl)piperidin-1- yl)methanone |
| I-89a | 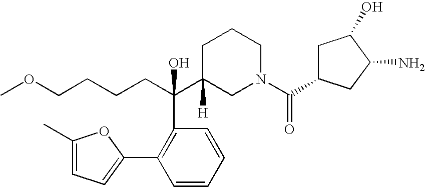 | ((1S,3R,4S)-3-amino-4- hydroxycyclopentyl)((R)-3- ((S)-1-hydroxy-5-methoxy-1- (2-(5-methylfuran-2- yl)phenyl)pentyl)piperidin-1- yl)methanone |
| I-90a | 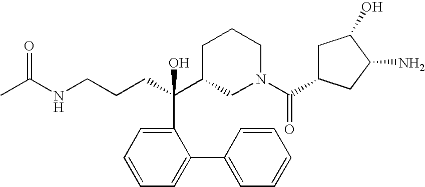 | N-((S)-4-((R)-1-((1S,3R,4S)- 3-amino-4- hydroxycyclopentanecarbonyl) piperidin-3-yl)-4-(biphenyl- 2-yl)-4- hydroxybutyl)acetamide |
| I-91a | 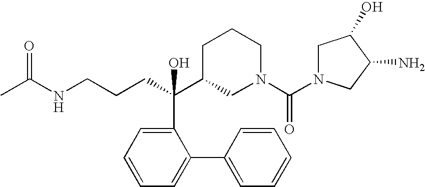 | N-((S)-4-((R)-1-((3R,4S)-3- amino-4-hydroxypyrrolidine- 1-carbonyl)piperidin-3-yl)-4- (biphenyl-2-yl)-4- hydroxybutyl)acetamide |
| I-92a | 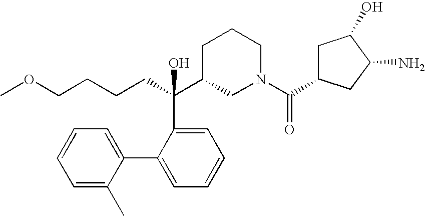 | ((1S,3R,4S)-3-amino-4- hydroxycyclopentyl)((3R)-3- ((1S)-1-hydroxy-5-methoxy- 1-(2′-methylbiphenyl-2- yl)pentyl)piperidin-1- yl)methanone |
| I-93a | 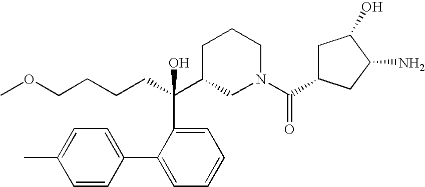 | ((1S,3R,4S)-3-amino-4- hydroxycyclopentyl)((3R)-3- ((S)-1-hydroxy-5-methoxy-1- (4′-methylbiphenyl-2- yl)pentyl)piperidin-1- yl)methanone |
| I-94a | 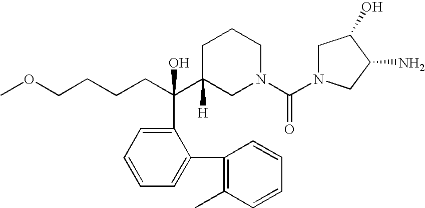 | ((3R,4S)-3-amino-4- hydroxypyrrolidin-1- yl)((3R)-3-((1S)-1-hydroxy- 5-methoxy-1-(2′- methylbiphenyl-2- yl)pentyl)piperidin-1- yl)methanone |
| I-95a | 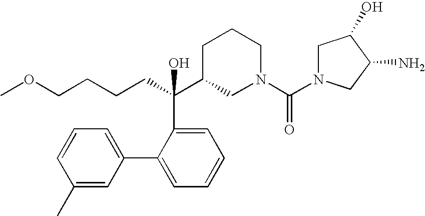 | ((3R,4S)-3-amino-4- hydroxypyrrolidin-1-yl)((R)- 3-((S)-1-hydroxy-5-methoxy- 1-(3′-methylbiphenyl-2- yl)pentyl)piperidin-1- yl)methanone |
| I-96a | 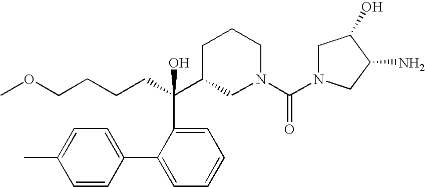 | ((3R,4S)-3-amino-4- hydroxypyrrolidin-1-yl)((R)- 3-((S)-1-hydroxy-5-methoxy- 1-(4′-methylbiphenyl-2- yl)pentyl)piperidin-1- yl)methanone |
| I-97a | 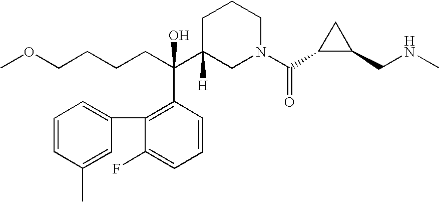 | ((R)-3-((S)-1-(6-fluoro-3′- methylbiphenyl-2-yl)-1- hydroxy-5- methoxypenlyl)piperidin-1- yl)((1R,2R)-2- ((methylamino)methyl)cyclo- propyl)methanone |
| I-98a | 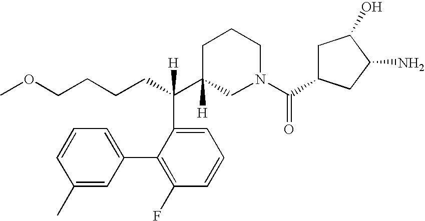 | ((1S,3R,4S)-3-amino-4- hydroxycyclopentyl)((S)-3- ((R)-1-(6-fluoro-3′- methylbiphenyl-2-yl)-5- methoxypentyl)piperidin-1- yl)methanone |
| I-99a | 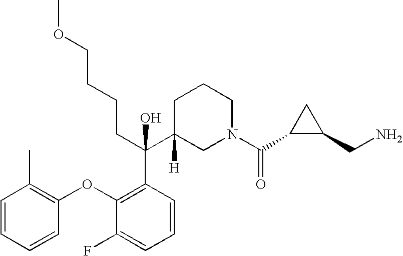 | ((1R,2R)-2- (aminomethyl)cyclopropyl)((R)- 3-((S)-1-(3-fluoro-2-(o- tolyloxy)phenyl)-1-hydroxy- 5-methoxypentyl)piperidin-1- yl)methanone |
| I-100a | 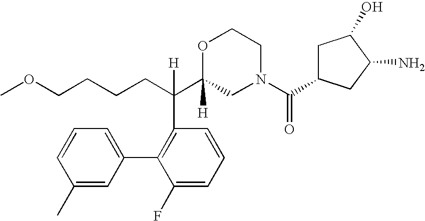 | ((1S,3R,4S)-3-amino-4- hydroxycyclopentyl)((S)-2- ((S)-1-(6-fluoro-3′- methylbiphenyl-2-yl)-5- methoxypentyl)morpholino) methanone |
| I-101a | 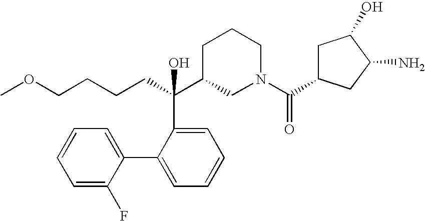 | ((1S,3R,4S)-3-amino-4- hydroxycyclopentyl)((3R)-3- ((1S)-1-(2′-fluorobiphenyl-2- yl)-1-hydroxy-5- methoxypentyl)piperidin-1- yl)methanone |
| I-102a | 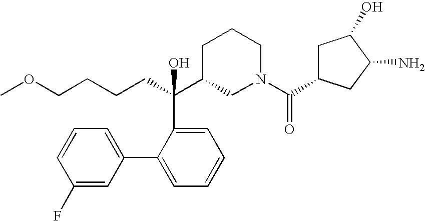 | ((1S,3R,4S)-3-amino-4- hydroxycyclopentyl)((R)-3- ((S)-1-(3′-fluorobiphenyl-2- yl)-1-hydroxy-5- methoxypentyl)piperidin-1- yl)methanone |
| I-103a | 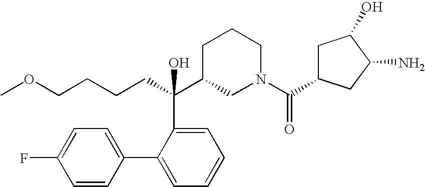 | ((1S,3R,4S)-3-amino-4- hydroxycyclopentyl)((R)-3- ((S)-1-(4′-fluorobiphenyl-2- yl)-1-hydroxy-5- methoxypentyl)piperidin-1- yl)methanone |
| I-104a | 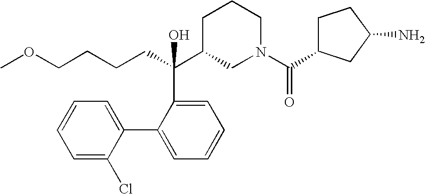 | ((1R,3S)-3- aminocyclopentyl)((3R)-3- ((1S)-1-(2′-chlorobiphenyl-2- yl)-1-hydroxy-5- methoxypentyl)piperidin-1- yl)methanone |
| I-105a | 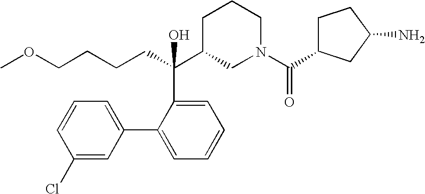 | ((1R,3S)-3- aminocyclopentyl)((R)-3- ((S)-1-(3′-chlorobiphenyl-2- yl)-1-hydroxy-5- methoxypentyl)piperidin-1- yl)methanone |
| I-106a | 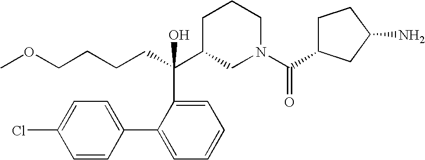 | ((1R,3S)-3- aminocyclopentyl)((R)-3- ((S)-1-(4′-chlorobiphenyl-2- yl)-1-hydroxy-5- methoxypentyl)piperidin-1- yl)methanone |
| I-107a | 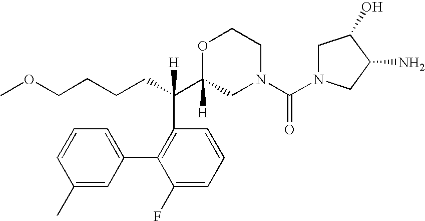 | ((3R,4S)-3-amino-4- hydroxypyrrolidin-1-yl)((S)- 2-((S)-1-(6-fluoro-3′- methylbiphenyl-2-yl)-5- methoxypentyl)morpholino) methanone |
| I-108a | 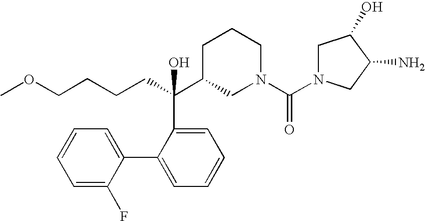 | ((3R,4S)-3-amino-4- hydroxypyrrolidin-1- yl)((3R)-3-((1S)-1-(2′- fluorobiphenyl-2-yl)-1- hydroxy-5- methoxypentyl)piperidin-1- yl)methanone |
| I-109a | 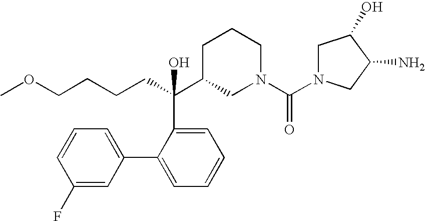 | ((3R,4S)-3-amino-4- hydroxypyrrolidin-1-yl)((R)- 3-((S)-1-(3′-fluorobiphenyl- 2-yl)-1-hydroxy-5- methoxypentyl)piperidin-1- yl)methanone |
| I-110a | 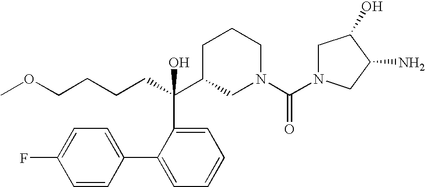 | ((3R,4S)-3-amino-4- hydroxypyrrolidin-1-yl)((R)- 3-((S)-1-(4′-fluorobiphenyl- 2-yl)-1-hydroxy-5- methoxypentyl)piperidin-1- yl)methanone |
| I-111a | 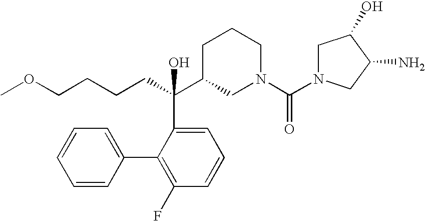 | ((3R,4S)-3-amino-4- hydroxypyrrolidin-1-yl)((R)- 3-((S)-1-(6-fluorobiphenyl-2- yl)-1-hydroxy-5- methoxypentyl)piperidin-1- yl)methanone |
| I-112a | 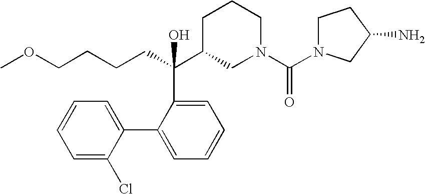 | ((S)-3-aminopyrrolidin-1- yl)((3R)-3-((1S)-1-(2′- chlorobiphenyl-2-yl)-1- hydroxy-5- methoxypentyl)piperidin-1- yl)methanone |
| I-113a | 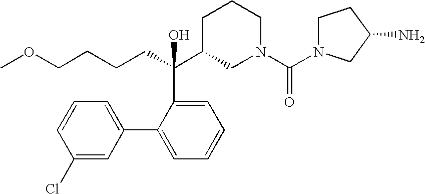 | ((S)-3-aminopyrrolidin-1- yl)((R)-3-((S)-1-(3′- chlorobiphenyl-2-yl)-1- hydroxy-5- methoxypentyl)piperidin-1- yl)methanone |
| I-114a | 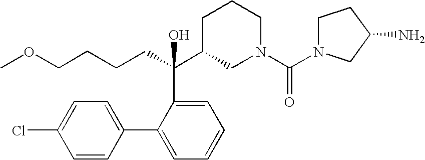 | ((S)-3-aminopyrrolidin-1- yl)((R)-3-((S)-1-(4′- chlorobiphenyl-2-yl)-1- hydroxy-5- methoxypentyl)piperidin-1- yl)methanone |
| I-115a | 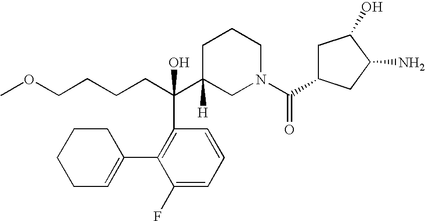 | ((1S,3R,4S)-3-amino-4- hydroxycyclopentyl)((R)-3- ((S)-1-(2-cyclohexenyl-3- fluorophenyl)-1-hydroxy-5- methoxypentyl)piperidin-1- yl)methanone |
| I-116a | 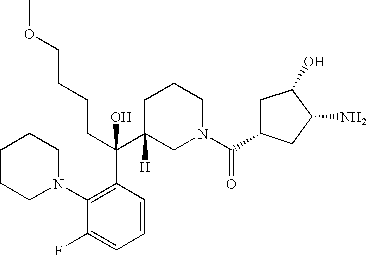 | ((1S,3R,4S)-3-amino-4- hydroxycyclopentyl)((R)-3- ((S)-1-(3-fluoro-2-(piperidin- 1-yl)phenyl)-1-hydroxy-5- methoxypentyl)piperidin-1- yl)methanone |
| I-117a | 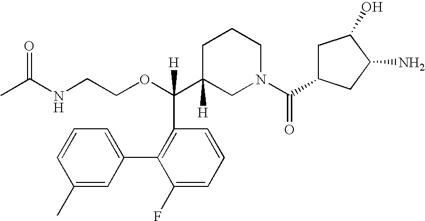 | N-(2-((R)-((R)-1- ((1S,3R,4S)-3-amino-4- hydroxycyclopentanecarbonyl) piperidin-3-yl)(6-fluoro-3′- methylbiphenyl-2- yl)methoxy)ethyl)acetamide |
| I-118a | 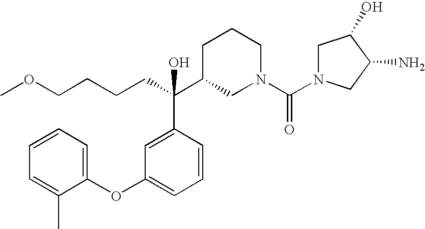 | ((3R,4S)-3-amino-4- hydroxypyrrolidin-1-yl)((R)- 3-((S)-1-hydroxy-5-methoxy- 1-(3-(o- tolyloxy)phenyl)pentyl) piperidin-1-yl)methanone |
| I-118b | 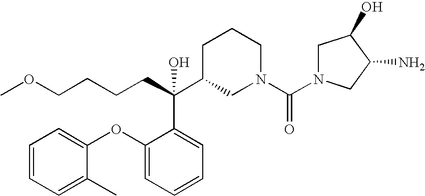 | ((3R,4R)-3-amino-4- hydroxypyrrolidin-1-yl)((R)- 3-((R)-1-hydroxy-5- methoxy-1-(2-(o- tolyloxy)phenyl)pentyl) piperidin-1-yl)methanone |
| I-118c | 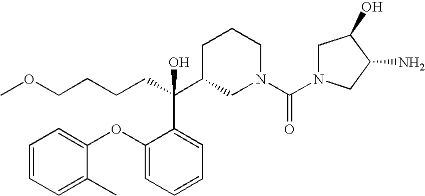 | ((3R,4R)-3-amino-4- hydroxypyrrolidin-1-yl)((R)- 3-((S)-1-hydroxy-5-methoxy- 1-(2-(o- tolyloxy)phenyl)pentyl) piperidin-1-yl)methanone |
| I-119a | 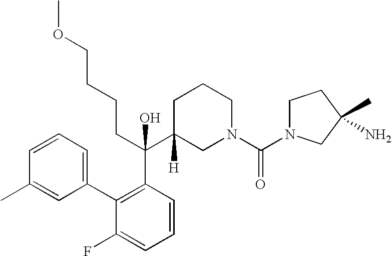 | ((S)-3-amino-3- methylpyrrolidin-1-yl)((R)-3- ((S)-1-(6-fluoro-3′- methylbiphenyl-2-yl)-1- hydroxy-5- methoxypentyl)piperidin-1- yl)methanone |
| I-120a | 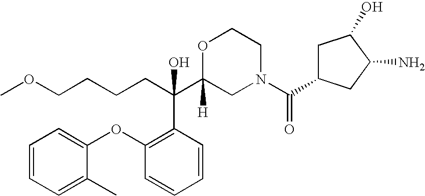 | ((1S,3R,4S)-3-amino-4- hydroxycyclopentyl)((R)-2- ((R)-1-hydroxy-5-methoxy- 1-(2-(o- tolyloxy)phenyl)pentyl) morpholino)methanone |
| I-121a | 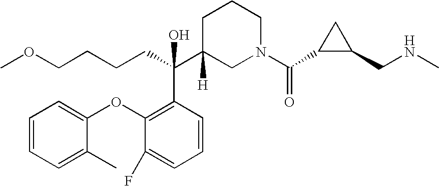 | ((R)-3-((S)-1-(3-fluoro-2-(o- tolyloxy)phenyl)-1-hydroxy- 5-methoxypentyl)piperidin-1- yl)((1R,2R)-2- ((methylamino)methyl) cyclopropyl)methanone |
| I-122a | 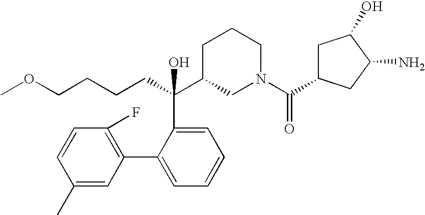 | ((1S,3R,4S)-3-amino-4- hydroxycyclopentyl)((3R)-3- ((1S)-1-(2′-fluoro-5′- methylbiphenyl-2-yl)-1- hydroxy-5- methoxypentyl)piperidin-1- yl)methanone |
| I-123a | 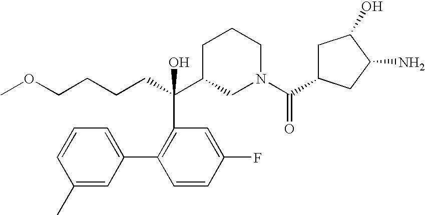 | ((1S,3R,4S)-3-amino-4- hydroxycyclopentyl)((R)-3- ((S)-1-(4-fluoro-3′- methylbiphenyl-2-yl)-1- hydroxy-5- methoxypentyl)piperidin-1- yl)methanone |
| I-124a | 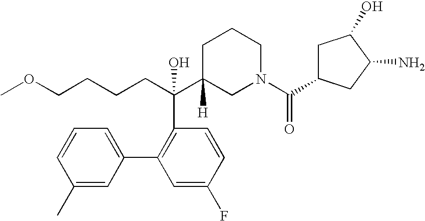 | ((1S,3R,4S)-3-amino-4- hydroxycyclopentyl)((R)-3- ((R)-1-(5-fluoro-3′- methylbiphenyl-2-yl)-1- hydroxy-5- methoxypentyl)piperidin-1- yl)methanone |
| I-125a | 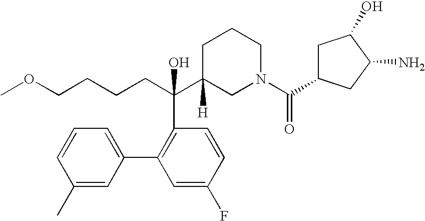 | ((1S,3R,4S)-3-amino-4- hydroxycyclopentyl)((R)-3- ((S)-1-(5-fluoro-3′- methylbiphenyl-2-yl)-1- hydroxy-5- methoxypentyl)piperidin-1- yl)methanone |
| I-126a | 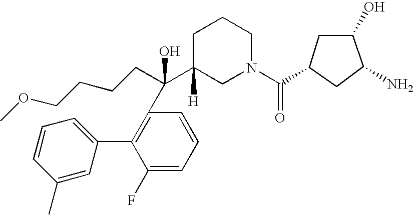 | ((1S,3R,4S)-3-amino-4- hydroxycyclopentyl)((R)-3- ((S)-1-(6-fluoro-3′- methylbiphenyl-2-yl)-1- hydroxy-5- methoxypentyl)piperidin-1- yl)methanone |
| I-127a | 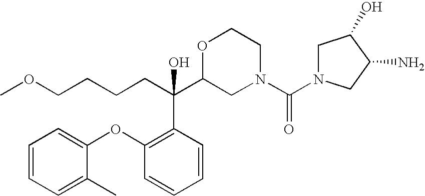 | ((3R,4S)-3-amino-4- hydroxypyrrolidin-1-yl)((R)- 2-((R)-1-hydroxy-5- methoxy-1-(2-(o- tolyloxy)phenyl)pentyl) morpholino)methanone |
| I-128a | 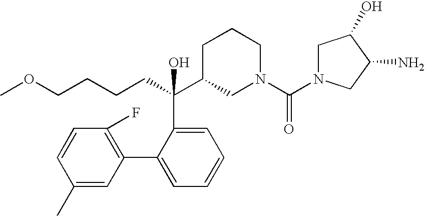 | ((3R,4S)-3-amino-4- hydroxypyrrolidin-1- yl)((3R)-3-((1S)-1-(2′-fluoro- 5′-methylbiphenyl-2-yl)-1- hydroxy-5- methoxypentyl)piperidin-1- yl)methanone |
| I-129a | 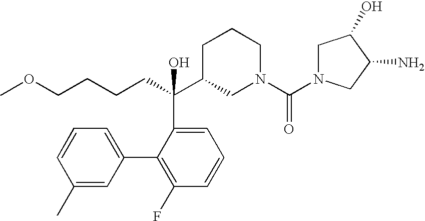 | ((3R,4S)-3-amino-4- hydroxypyrrolidin-1-yl)((R)- 3-((S)-1-(6-fluoro-3′- methylbiphenyl-2-yl)-1- hydroxy-5- methoxypentyl)piperidin-1- yl)methanone |
| I-129b | 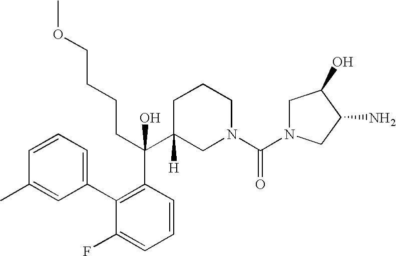 | ((3R,4R)-3-amino-4- hydroxypyrrolidin-1-yl)((R)- 3-((S)-1-(6-fluoro-3′- methylbiphenyl-2-yl)-1- hydroxy-5- methoxypentyl)piperidin-1- yl)methanone |
| I-130a | 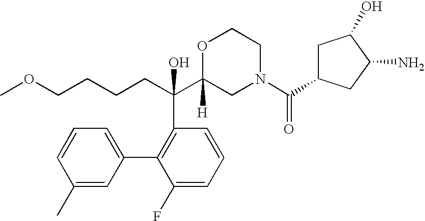 | ((1S,3R,4S)-3-amino-4- hydroxycyclopentyl)((R)-2- ((R)-1-(6-fluoro-3′- methylbiphenyl-2-yl)-1- hydroxy-5- methoxypentyl)morpholino) methanone |
| I-131a | 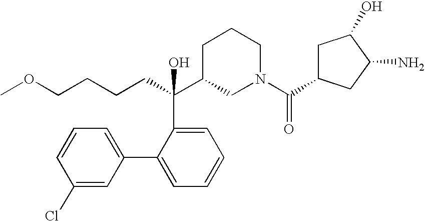 | ((1S,3R,4S)-3-amino-4- hydroxycyclopentyl)((R)-3- ((S)-1-(3′-chlorobiphenyl-2- yl)-1-hydroxy-5- methoxypentyl)piperidin-1- yl)methanone |
| I-132a | 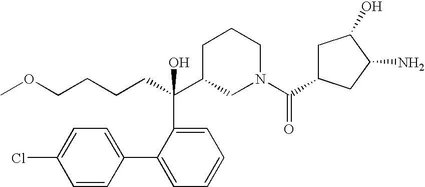 | ((1S,3R,4S)-3-amino-4- hydroxycyclopentyl)((R)-3- ((S)-1-(4′-chlorobiphenyl-2- yl)-1-hydroxy-5- methoxypentyl)piperidin-1- yl)methanone |
| I-133a | 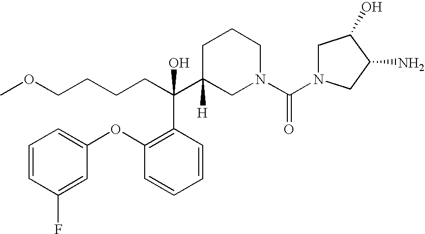 | ((3R,4S)-3-amino-4- hydroxypyrrolidin-1-yl)((R)- 3-((S)-1-(2-(3- fluorophenoxy)phenyl)-1- hydroxy-5- methoxypentyl)piperidin-1- yl)methanone |
| I-134a | 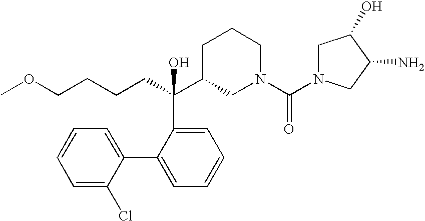 | ((3R,4S)-3-amino-4- hydroxypyrrolidin-1- yl)((3R)-3-((1S)-1-(2′- chlorobiphenyl-2-yl)-1- hydroxy-5- methoxypentyl)piperidin-1- yl)methanone |
| I-135a | 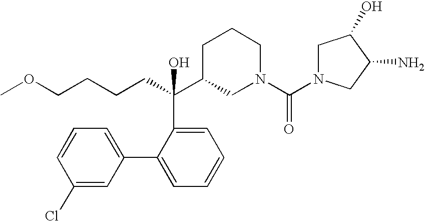 | ((3R,4S)-3-amino-4- hydroxypyrrolidin-1-yl)((R)- 3-((S)-1-(3′-chlorobiphenyl- 2-yl)-1-hydroxy-5- methoxypentyl)piperidin-1- yl)methanone |
| I-136a | 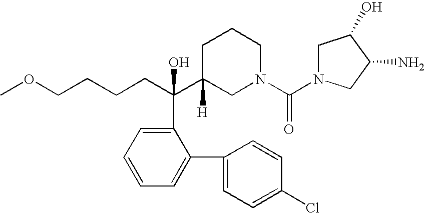 | ((3R,4S)-3-amino-4- hydroxypyrrolidin-1-yl)((R)- 3-((S)-1-(4′-chlorobiphenyl- 2-yl)-1-hydroxy-5- methoxypentyl)piperidin-1- yl)methanone |
| I-137a | 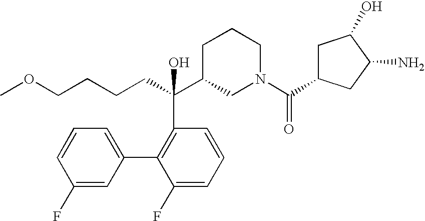 | ((1S,3R,4S)-3-amino-4- hydroxycyclopentyl)((R)-3- ((S)-1-(3′,6-difluorobiphenyl- 2-yl)-1-hydroxy-5- methoxypentyl)piperidin-1- yl)methanone |
| I-138a | 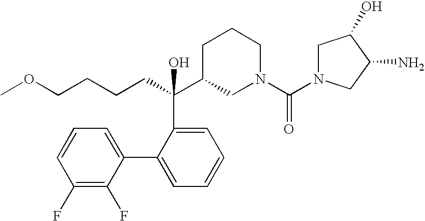 | ((3R,4S)-3-amino-4- hydroxypyrrolidin-1- yl)((3R)-3-((1S)-1-(2′,3′- difluorobiphenyl-2-yl)-1- hydroxy-5- methoxypentyl)piperidin-1- yl)methanone |
| I-139a | 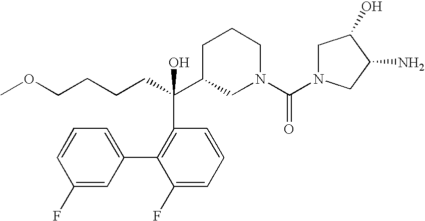 | ((3R,4S)-3-amino-4- hydroxypyrrolidin-1-yl)((R)- 3-((S)-1-(3′,6- difluorobiphenyl-2-yl)-1- hydroxy-5- methoxypentyl)piperidin-1- yl)methanone |
| I-140a | 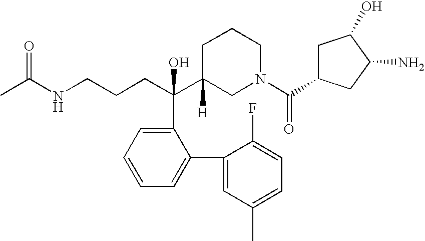 | N-((4S)-4-((R)-1- ((1S,3R,4S)-3-amino-4- hydroxycyclopentanecarbonyl) piperidin-3-yl)-4-(2′-fluoro- 5′-methylbiphenyl-2-yl)-4- hydroxybutyl)acetamide |
| I-141a | 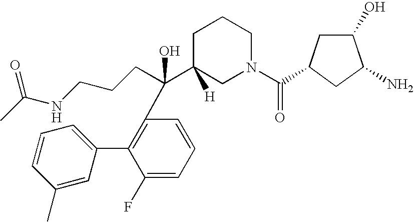 | N-((S)-4-((R)-1-((1S,3R,4S)- 3-amino-4- hydroxycyclopentanecarbonyl) piperidin-3-yl)-4-(6-fluoro- 3′-methylbiphenyl-2-yl)-4- hydroxybutyl)acetamide |
| I-142a | 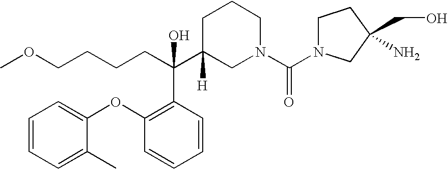 | ((S)-3-amino-3- (hydroxymethyl)pyrrolidin-1- yl)((R)-3-((S)-1-hydroxy-5- methoxy-1-(2-(o- tolyloxy)phenyl)pentyl) piperidin-1-yl)methanone |
| I-143a | 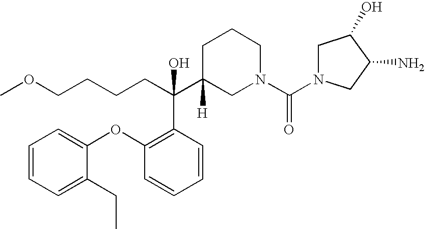 | ((3R,4S)-3-amino-4- hydroxypyrrolidin-1-yl)((R)- 3-((S)-1-(2-(2- ethylphenoxy)phenyl)-1- hydroxy-5- methoxypentyl)piperidin-1- yl)methanone |
| I-144a | 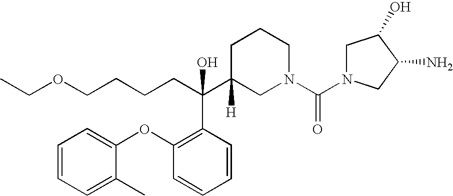 | ((3R,4S)-3-amino-4- hydroxypyrrolidin-1-yl)((R)- 3-((S)-5-ethoxy-1-hydroxy-1- (2-(o-tolyloxy)phenyl)pentyl) piperidin-1-yl)methanone |
| I-145a | 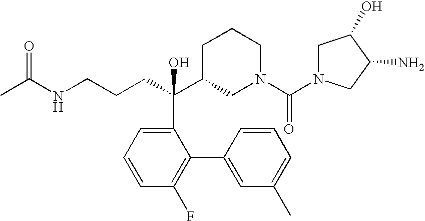 | N-((S)-4-((R)-1-((3R,4S)-3- amino-4-hydroxypyrrolidine- 1-carbonyl)piperidin-3-yl)-4- (6-fluoro-3′-methylbiphenyl- 2-yl)-4- hydroxybutyl)acetamide |
| I-146a | 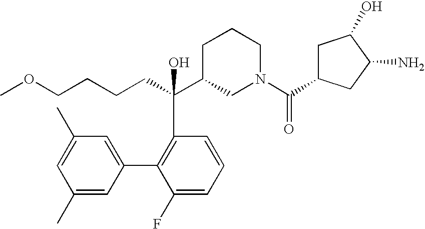 | ((1S,3R,4S)-3-amino-4- hydroxycyclopentyl)((R)-3- ((S)-1-(6-fluoro-3′,5′- dimethylbiphenyl-2-yl)-1- hydroxy-5- methoxypentyl)piperidin-1- yl)methanone |
| I-147a | 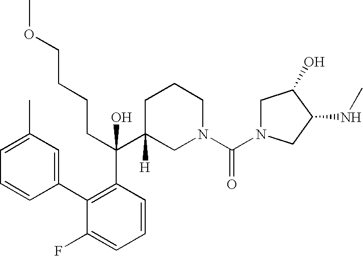 | ((R)-3-((S)-1-(6-fluoro-3′- methylbiphenyl-2-yl)-1- hydroxy-5- methoxypentyl)piperidin-1- yl)((3S,4R)-3-hydroxy-4- (methylamino)pyrrolidin-1- yl)methanone |
| I-148a | 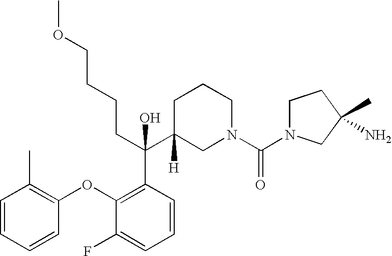 | ((S)-3-amino-3- methylpyrrolidin-1-yl)((R)-3- ((S)-1-(3-fluoro-2-(o- tolyloxy)phenyl)-1-hydroxy- 5-methoxypentyl)piperidin-1- yl)methanone |
| I-149a | 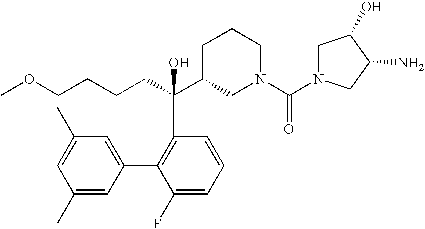 | ((3R,4S)-3-amino-4- hydroxypyrrolidin-1-yl)((R)- 3-((S)-1-(6-fluoro-3′,5′- dimethylbiphenyl-2-yl)-1- hydroxy-5- methoxypentyl)piperidin-1- yl)methanone |
| I-150a | 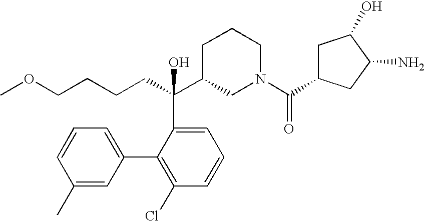 | ((1S,3R,4S)-3-amino-4- hydroxycyclopentyl)((R)-3- ((S)-1-(6-chloro-3′- methylbiphenyl-2-yl)-1- hydroxy-5- methoxypentyl)piperidin-1- yl)methanone |
| I-151a | 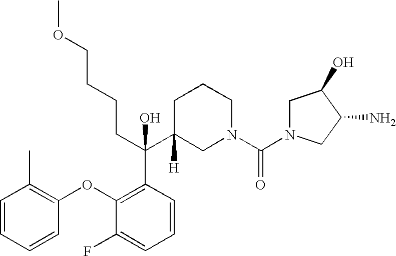 | ((3R,4R)-3-amino-4- hydroxypyrrolidin-1-yl)((R)- 3-((S)-1-(3-fluoro-2-(o- tolyloxy)phenyl)-1-hydroxy- 5-methoxypentyl)piperidin-1- yl)methanone |
| I-152a | 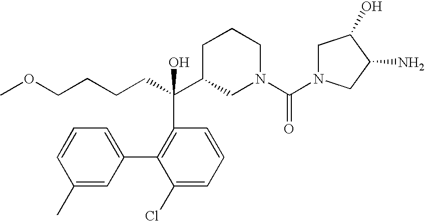 | ((3R,4S)-3-amino-4- hydroxypyrrolidin-1-yl)((R)- 3-((S)-1-(6-chloro-3′- methylbiphenyl-2-yl)-1- hydroxy-5- methoxypentyl)piperidin-1- yl)methanone |
| I-153a | 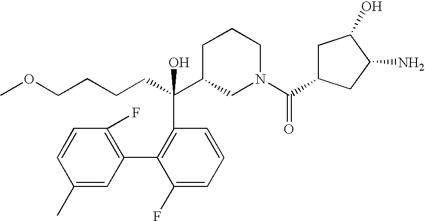 | ((1S,3R,4S)-3-amino-4- hydroxycyclopentyl)((3R)-3- ((1S)-1-(2′,6-difluoro-5′- methylbiphenyl-2-yl)-1- hydroxy-5- methoxypentyl)piperidin-1- yl)methanone |
| I-154a | 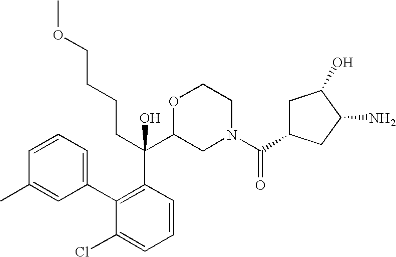 | ((1S,3R,4S)-3-amino-4- hydroxycyclopentyl)(2-((R)- 1-(6-chloro-3′- methylbiphenyl-2-yl)-1- hydroxy-5- methoxypentyl)morpholino) methanone |
| I-155a | 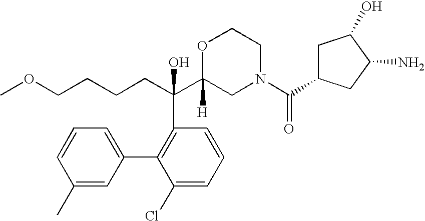 | ((1S,3R,4S)-3-amino-4- hydroxycyclopentyl)((R)-2- ((R)-1-(6-chloro-3′- methylbiphenyl-2-yl)-1- hydroxy-5- methoxypentyl)morpholino) methanone |
| I-156a | 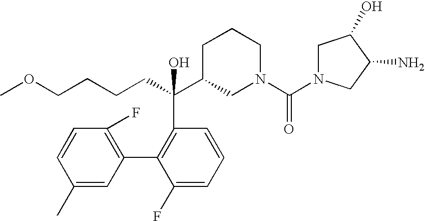 | ((3R,4S)-3-amino-4- hydroxypyrrolidin-1- yl)((3R)-3-((1S)-1-(2′,6- difluoro-5′-methylbiphenyl- 2-yl)-1-hydroxy-5- methoxypentyl)piperidin-1- yl)methanone |
| I-157a | 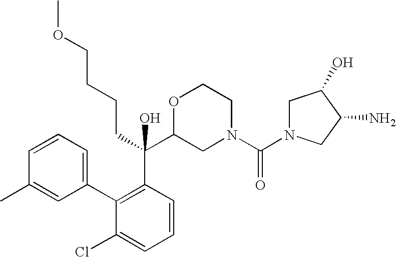 | ((3R,4S)-3-amino-4- hydroxypyrrolidin-1- yl)((RS)-2-((RS)-1-(6-chloro- 3′-methylbiphenyl-2-yl)-1- hydroxy-5- methoxypentyl)morpholino) methanone |
| I-158a | 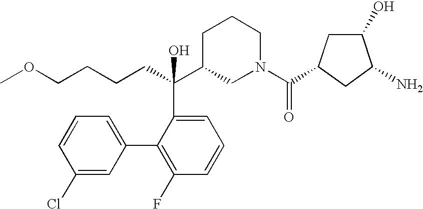 | ((1S,3R,4S)-3-amino-4- hydroxycyclopentyl)((R)-3- ((S)-1-(3′-chloro-6- fluorobiphenyl-2-yl)-1- hydroxy-5- methoxypentyl)piperidin-1- yl)methanone |
| I-159a | 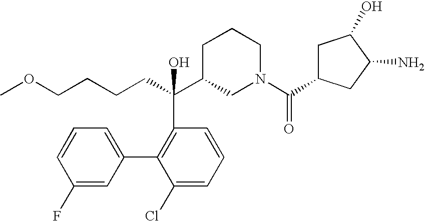 | ((1S,3R,4S)-3-amino-4- hydroxycyclopentyl)((R)-3- ((S)-1-(6-chloro-3′- fluorobiphenyl-2-yl)-1- hydroxy-5- methoxypentyl)piperidin-1- yl)methanone |
| I-160a | 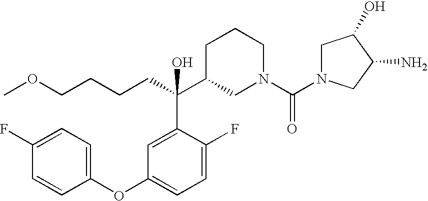 | ((3R,4S)-3-amino-4- hydroxypyrrolidin-1-yl)((R)- 3-((S)-1-(2-fluoro-5-(4- fluorophenoxy)phenyl)-1- hydroxy-5- methoxypentyl)piperidin-1- yl)methanone |
| I-161a | 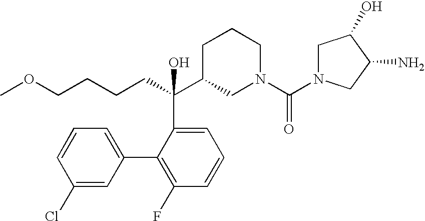 | ((3R,4S)-3-amino-4- hydroxypyrrolidin-1-yl)((R)- 3-((S)-1-(3′-chloro-6- fluorobiphenyl-2-yl)-1- hydroxy-5- methoxypentyl)piperidin-1- yl)methanone |
| I-162a | 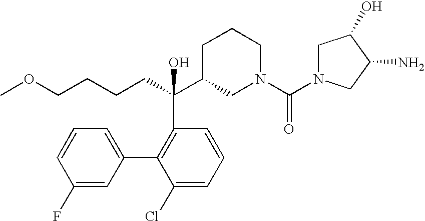 | ((3R,4S)-3-amino-4- hydroxypyrrolidin-1-yl)((R)- 3-((S)-1-(6-chloro-3′- fluorobiphenyl-2-yl)-1- hydroxy-5- methoxypentyl)piperidin-1- yl)methanone |
| I-163a | 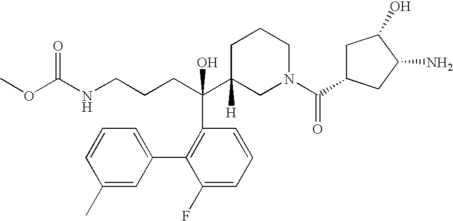 | methyl (S)-4-((R)-1- ((1S,3R,4S)-3-amino-4- hydroxycyclopentanecarbonyl) piperidin-3-yl)-4-(6-fluoro- 3′-methylbiphenyl-2-yl)-4- hydroxybutylcarbamate |
| I-164a | 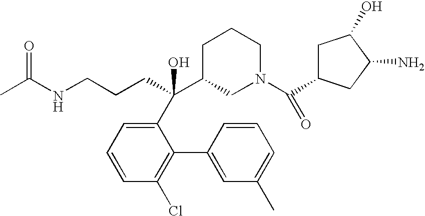 | N-((S)-4-((R)-1-((1S,3R,4S)- 3-amino-4- hydroxycyclopentanecarbonyl) piperidin-3-yl)-4-(6-chloro- 3′-methylbiphenyl-2-yl)-4- hydroxybutyl)acetamide |
| I-165a | 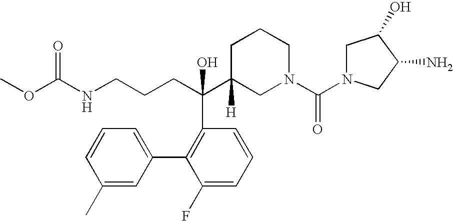 | methyl (S)-4-((R)-1- ((3R,4S)-3-amino-4- hydroxypyrrolidine-1- carbonyl)piperidin-3-yl)-4- (6-fluoro-3′-methylbiphenyl- 2-yl)-4- hydroxybutylcarbamate |
| I-165b | 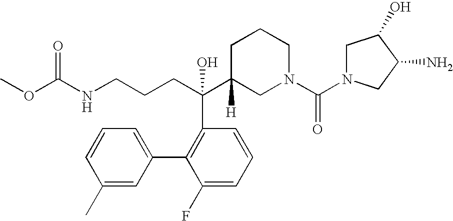 | methyl (R)-4-((R)-1- ((3R,4S)-3-amino-4- hydroxypyrrolidine-1- carbonyl)piperidin-3-yl)-4- (6-fluoro-3′-methylbiphenyl- 2-yl)-4- hydroxybutylcarbamate |
| I-166a | 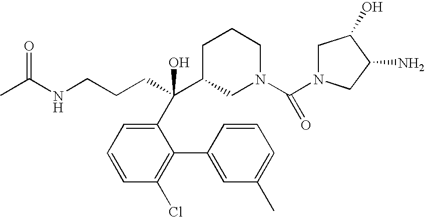 | N-((S)-4-((R)-1-((3R,4S)-3- amino-4-hydroxypyrrolidine- 1-carbonyl)piperidin-3-yl)-4- (6-chloro-3′-methylbiphenyl- 2-yl)-4- hydroxybutyl)acetamide |
| I-167a | 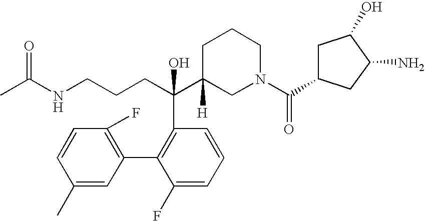 | N-((4S)-4-((R)-1- ((1S,3R,4S)-3-amino-4- hydroxycyclopentanecarbonyl) piperidin-3-yl)-4-(2′,6- difluoro-5′-methylbiphenyl- 2-yl)-4- hydroxyburyl)acetamide |
| I-168a | 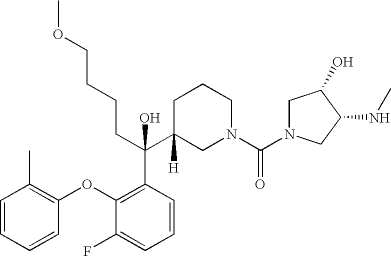 | ((R)-3-((S)-1-(3-fluoro-2-(o- tolyloxy)phenyl)-1-hydroxy- 5-methoxypentyl)piperidin-1- yl)((3S,4R)-3-hydroxy-4- (methylamino)pyrrolidin-1- yl)methanone |
| I-169a | 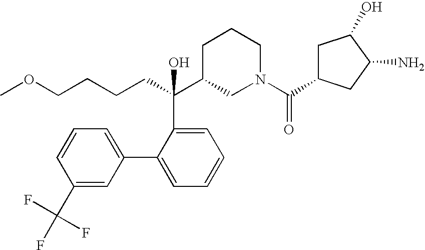 | ((1S,3R,4S)-3-amino-4- hydroxycyclopentyl)((R)-3- ((S)-1-hydroxy-5-methoxy-1- (3′-(trifluoromethyl)biphenyl- 2-yl)pentyl)piperidin-1- yl)methanone |
| I-170a | 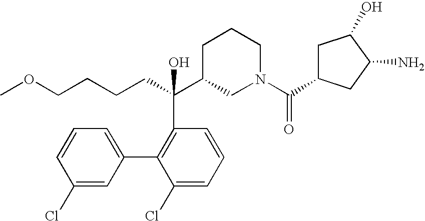 | ((1S,3R,4S)-3-amino-4- hydroxycyclopentyl)((R)-3- ((S)-1-(3′,6- dichlorobiphenyl-2-yl)-1- hydroxy-5- methoxypentyl)piperidin-1- yl)methanone |
| I-171a | 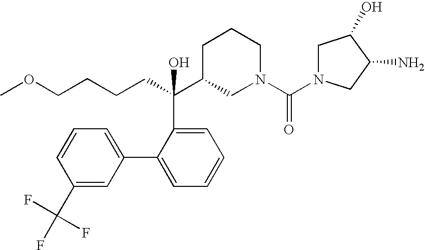 | ((3R,4S)-3-amino-4- hydroxypyrrolidin-1-yl)((R)- 3-((S)-1-hydroxy-5-methoxy- 1-(3′- (trifluoromethyl)biphenyl-2- yl)pentyl)piperidin-1- yll)methanone |
| I-172a | 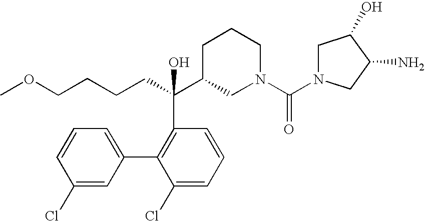 | ((3R,4S)-3-amino-4- hydroxypyrrolidin-1-yl)((R)- 3-((S)-1-(3′,6- dichlorobiphenyl-2-yl)-1- hydroxy-5- methoxypentyl)piperidin-1- yl)methanone |
| I-173a | 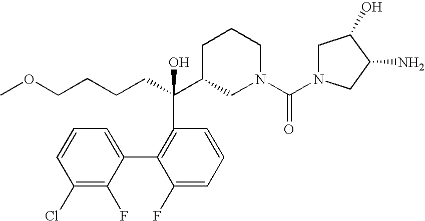 | ((1S,3R,4S)-3-amino-4- hydroxycyclopentyl)((3R)-3- ((1R)-1-(3′-chloro-2′,6- difluorobiphenyl-2-yl)-1- hydroxy-5- methoxypentyl)piperidin-1- yl)methanone |
| I-173b | 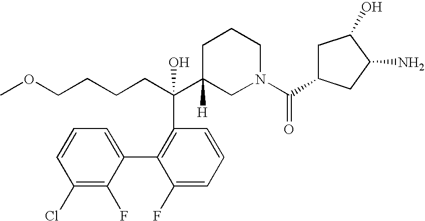 | ((1S,3R,4S)-3-amino-4- hydroxycyclopentyl)((3R)-3- ((1R)-1-(3′-chloro-2′,6- difluorobiphenyl-2-yl)-1- hydroxy-5- methoxypentyl)piperidin-1- yl)methanone |
| I-174a | 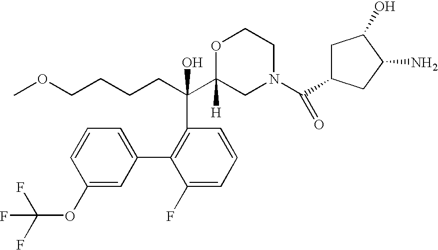 | ((1S,3R,4S)-3-amino-4- hydroxycyclopentyl)((R)-2- ((R)-1-(6-fluoro-3′- (trifluoromethoxy)biphenyl- 2-yl)-1-hydroxy-5- methoxypentyl)morpholino) methanone |
| I-175a | 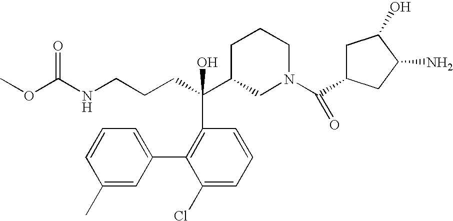 | methyl (S)-4-((R)-1- ((1S,3R,4S)-3-amino-4- hydroxycyclopentanecarbonyl) piperidin-3-yl)-4-(6-chloro- 3′-methylbiphenyl-2-yl)-4- hydroxybutylcarbamate |
| I-176a | 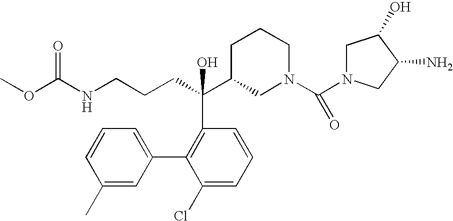 | methyl (S)-4-((R)-1- ((3R,4S)-3-amino-4- hydroxypyrrolidine-1- carbonyl)piperidin-3-yl)-4- (6-chloro-3′-methylbiphenyl- 2-yl)-4- hydroxybutylcarbamate and |
| I-177a | 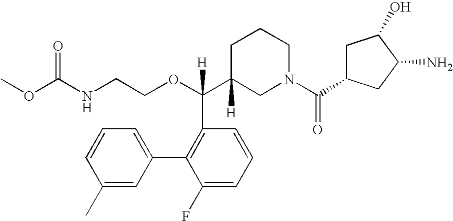 | methyl 2-((R)-((R)-1- ((1S,3R,4S)-3-amino-4- hydroxycyclopentanecarbonyl) piperidin-3-yl)(6-fluoro-3′- methylbiphenyl-2- yl)methoxy)ethylcarbamate |
| I-177a | 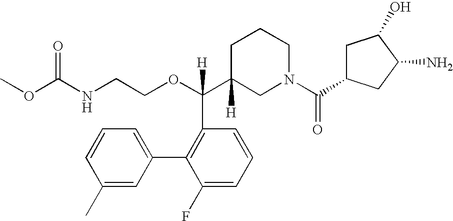 | methyl 2-((R)-((R)-1- ((1S,3R,4S)-3-amino-4- hydroxycyclopentanecarbonyl) piperidin-3-yl)(6-fluoro-3′- methylbiphenyl-2- yl)methoxy)ethylcarbamate |
| I-178a | 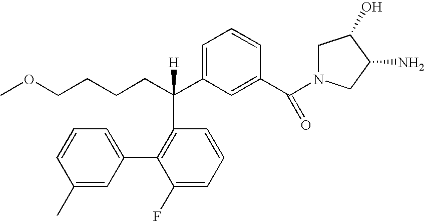 | ((3R,4S)-3-amino-4- hydroxypyrrolidin-1-yl)(3- ((R)-1-(6-fluoro-3′- methylbiphenyl-2-yl)-5- methoxypentyl)phenyl) methanone |
| I-178b | 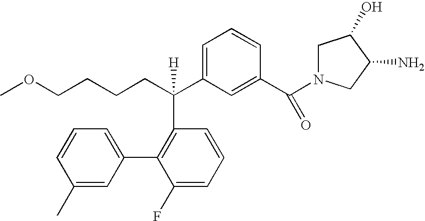 | ((3R,4S)-3-amino-4- hydroxypyrrolidin-1-yl)(3- ((S)-1-(6-fluoro-3′- methylbiphenyl-2-yl)-5- methoxypentyl)phenyl) methanone |
| I-179a | 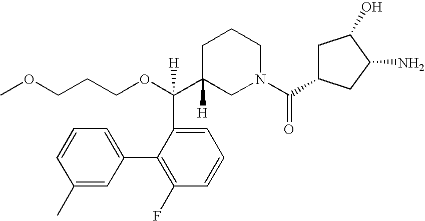 | ((1S,3R,4S)-3-amino-4- hydroxycyclopentyl)((R)-3- ((S)-(6-fluoro-3′- methylbiphenyl-2-yl)(3- methoxypropoxy)methyl) piperidin-1-yl)methanone |
| I-180a | 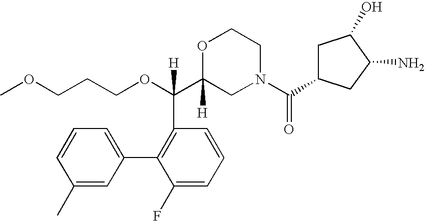 | ((1S,3R,4S)-3-amino-4- hydroxycyclopentyl)((R)-2- ((S)-(6-fluoro-3′- methylbiphenyl-2-yl)(3- methoxypropoxy)methyl) morpholino)methanone |
| I-181a | 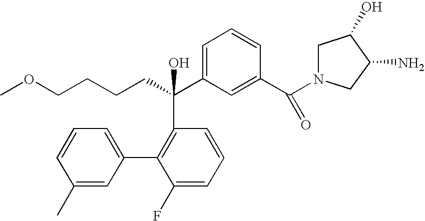 | ((3R,4S)-3-amino-4- hydroxypyrrolidin-1-yl)(3- ((S)-1-(6-fluoro-3′- methylbiphenyl-2-yl)-1- hydroxy-5- methoxypentyl)phenyl) methanone |
| I-182a | 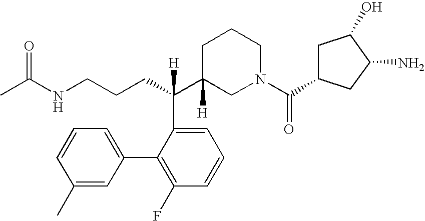 | N-((R)-4-((S)-1-((1S,3R,4S)- 3-amino-4- hydroxycyclopentanecarbonyl) piperidin-3-yl)-4-(6-fluoro- 3′-methylbiphenyl-2- yl)butyl)acetamide |
| I-183a | 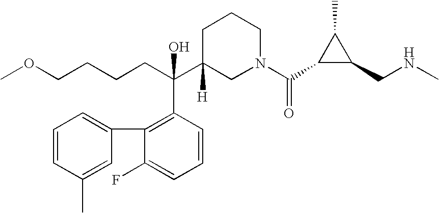 | ((R)-3-((S)-1-(6-fluoro-3′- methylbiphenyl-2-yl)-1- hydroxy-5- methoxypentyl)piperidin-1- yl)((1R,2R,3R)-2-methyl-3- ((methylamino)methyl) cyclopropyl)methanone |
| I-184a | 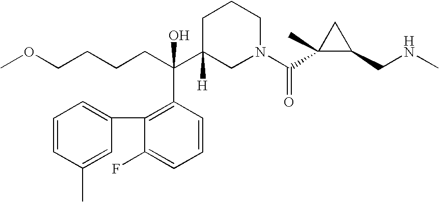 | ((R)-3-((S)-1-(6-fluoro-3′- methylbiphenyl-2-yl)-1- hydroxy-5- methoxypentyl)piperidin-1- yl)((1R,2R)-1-methyl-2- ((methylamino)methyl) cyclopropyl)methanone |
| I-185a | 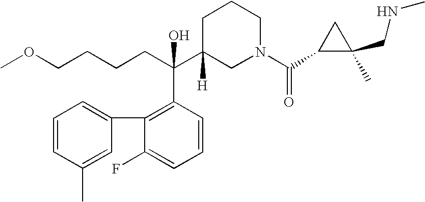 | ((R)-3-((S)-1-(6-fluoro-3′- methylbiphenyl-2-yl)-1- hydroxy-5- methoxypentyl)piperidin-1- yl)((1R,2R)-2-methyl-2- ((methylamino)methyl) cyclopropyl)methanone |
| I-186a | 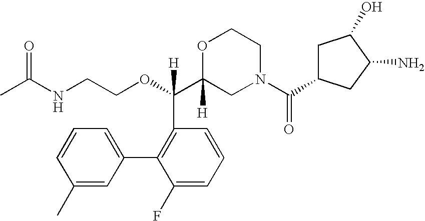 | N-(2-((S)-((R)-4- ((1S,3R,4S)-3-amino-4- hydroxycyclopentanecarbonyl) morpholin-2-yl)(6-fluoro- 3′-methylbiphenyl-2- yl)methoxy)ethyl)acetamide |
| I-187a | 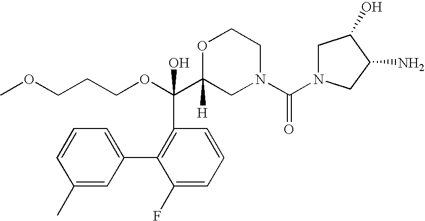 | ((3R,4S)-3-amino-4- hydroxypyrrolidin-1-yl)((R)- 2-((R)-1-(6-fluoro-3′- methylbiphenyl-2-yl)-1- hydroxy-5- methoxypentyl)morpholino) methanone |
| I-188a | 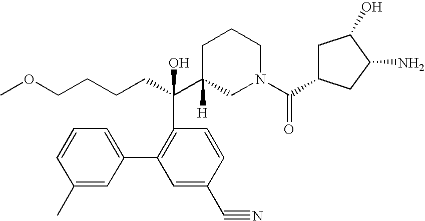 | 6-((S)-1-((R)-1-((1S,3R,4S)- 3-amino-4- hydroxycyclopentanecarbonyl) piperidin-3-yl)-1-hydroxy- 5-methoxypentyl)-3′- methylbiphenyl-3- carbonitrile |
| I-189a | 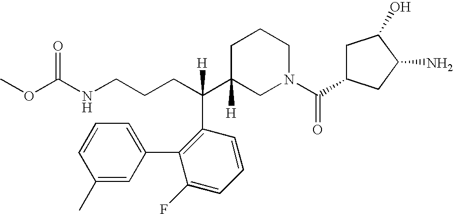 | methyl (R)-4-((S)-1- ((1S,3R,4S)-3-amino-4- hydroxycyclopentanecarbonyl) piperidin-3-yl)-4-(6-fluoro- 3′-methylbiphenyl-2- yl)butylcarbamate |
| I-189b | 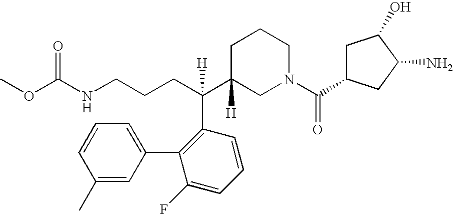 | methyl (S)-4-((S)-1- ((1S,3R,4S)-3-amino-4- hydroxycyclopentanecarbonyl) piperidin-3-yl)-4-(6-fluoro- 3′-methylbiphenyl-2- yl)butylcarbamate |
| I-190a | 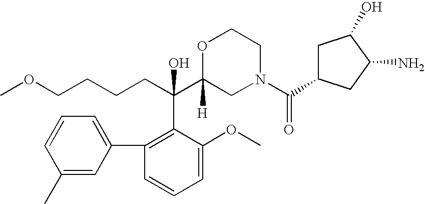 | ((1S,3R,4S)-3-amino-4- hydroxycyclopentyl)((R)-2- ((R)-1-hydroxy-5-methoxy- 1-(3-methoxy-3′- methylbipbenyl-2- yl)pentyl)morpholino) methanone |
| I-191a | 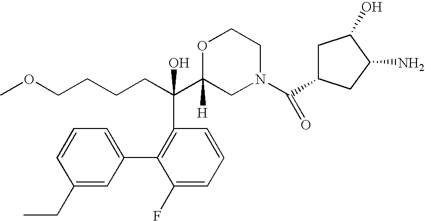 | ((1S,3R,4S)-3-amino-4- hydroxycyclopentyl)((R)-2- ((R)-1-(3′-ethyl-6- fluorobiphenyl-2-yl)-1- hydroxy-5- methoxypentyl)morpholino) methanone |
| I-192a | 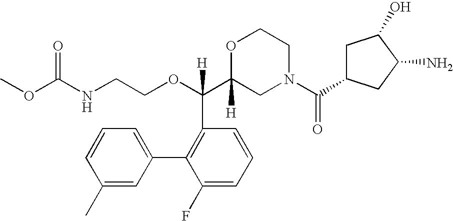 | methyl 2-((S)-((R)-4- ((1S,3R,4S)-3-amino-4- hydroxycyclopentanecarbonyl) morpholin-2-yl)(6-fluoro- 3′-methylbiphenyl-2- yl)methoxy)ethylcarbamate |
| I-193a | 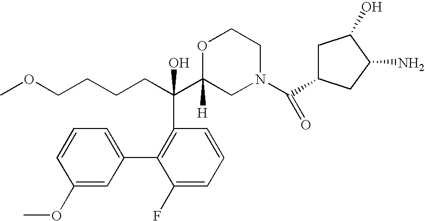 | ((1S,3R,4S)-3-amino-4- hydroxycyclopentyl)((R)-2- ((R)-1-(6-fluoro-3′- methoxybiphenyl-2-yl)-1- hydroxy-5- methoxypentyl)morpholino) methanone |
| I-193b | 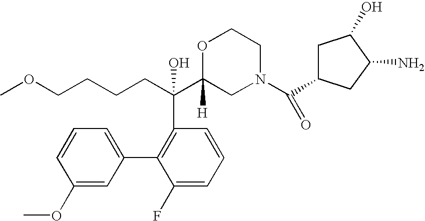 | ((1S,3R,4S)-3-amino-4- hydroxycyclopentyl)((R)-2- ((S)-1-(6-fluoro-3′- methoxybiphenyl-2-yl)-1- hydroxy-5- methoxypentyl)morpholino) methanone |
| I-194a | 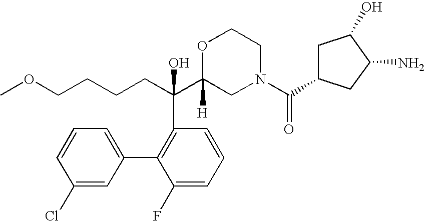 | ((1S,3R,4S)-3-amino-4- hydroxycyclopentyl)((R)-2- ((R)-1-(3′-chloro-6- fluorobiphenyl-2-yl)-1- hydroxy-5- methoxypentyl)morpholino) methanone |
| I-195a | 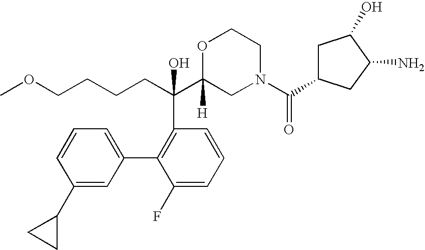 | ((1S,3R,4S)-3-amino-4- hydroxycyclopentyl)((R)-2- ((R)-1-(3′-cyclopropyl-6- fluorobiphenyl-2-yl)-1- hydroxy-5- methoxypentyl)morpholino) methanone |
| I-196a | 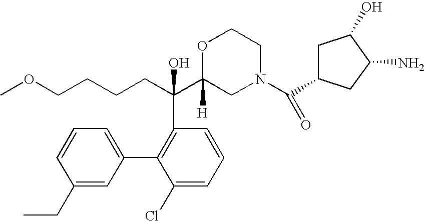 | ((1S,3R,4S)-3-amino-4- hydroxycyclopentyl)((R)-2- ((R)-1-(6-chloro-3′- ethylbiphenyl-2-yl)-1- hydroxy-5- methoxypentyl)morpholino) methanone |
| I-197a | 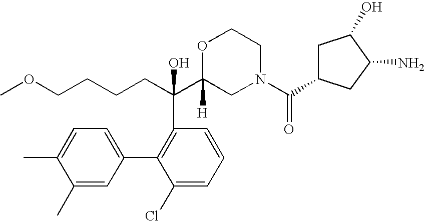 | ((1S,3R,4S)-3-amino-4- hydroxycyclopentyl)((R)-2- ((R)-1-(6-chloro-3′,4′- dimethylbiphenyl-2-yl)-1- hydroxy-5- methoxypentyl)morpholino) methanone |
| I-198a | 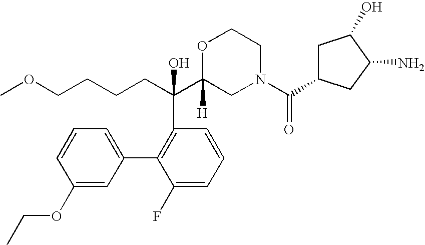 | ((1S,3R,4S)-3-amino-4- hydroxycyclopentyl)((R)-2- ((R)-1-(3′-ethoxy-6- fluorobiphenyl-2-yl)-1- hydroxy-5- methoxypentyl)morpholino) methanone |
| I-199a | 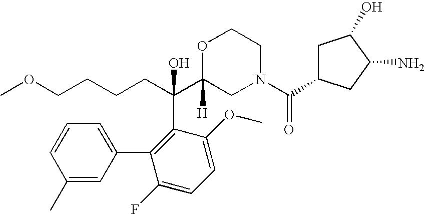 | ((1S,3R,4S)-3-amino-4- hydroxycyclopentyl)((R)-2- ((R)-1-(6-fluoro-3-methoxy- 3′-methylbiphenyl-2-yl)-1- hydroxy-5- methoxypentyl)morpholino) methanone |
| I-200a | 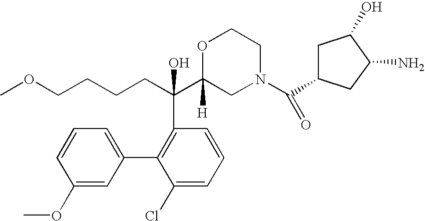 | ((1S,3R,4S)-3-amino-4- hydroxycyclopentyl)((R)-2- ((R)-1-(6-chloro-3′- methoxybiphenyl-2-yl)-1- hydroxy-5- methoxypentyl)morpholino) methanone |
| I-201a | 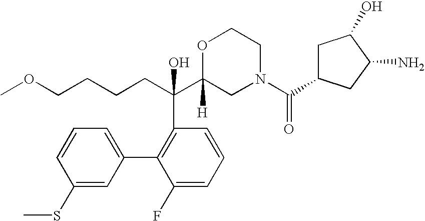 | ((1S,3R,4S)-3-amino-4- hydroxycyclopentyl)((R)-2- ((R)-1-(6-fluoro-3′- (methylthio)biphenyl-2-yl)-1- hydroxy-5- methoxypentyl)morpholino) methanone |
| I-201b | 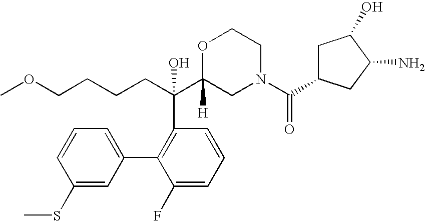 | ((1S,3R,4S)-3-amino-4- hydroxycyclopentyl)((R)-2- ((S)-1-(6-fluoro-3′- (methylthio)biphenyl-2-yl)-1- hydroxy-5- methoxypentyl)morpholino) methanone |
| I-202a | 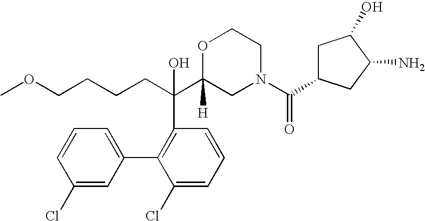 | ((1S,3R,4S)-3-amino-4- hydroxycyclopentyl)((R)-2- ((R)-1-(3′,6- dichlorobipbenyl-2-yl)-1- hydroxy-5- methoxypentyl)morpholino) methanone |
| I-203a | 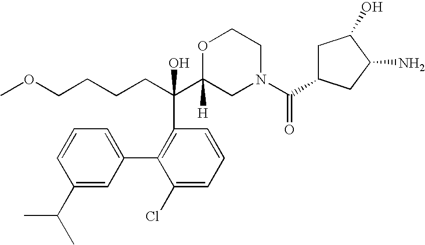 | ((1S,3R,4S)-3-amino-4- hydroxycyclopentyl)((R)-2- ((R)-1-(6-chloro-3′- isopropylbiphenyl-2-yl)-1- hydroxy-5- methoxypentyl)morpholino) methanone |
| I-204a | 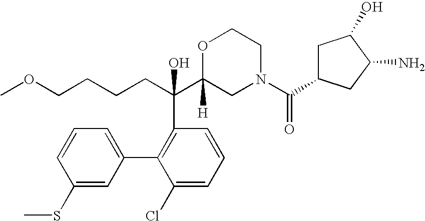 | ((1S,3R,4S)-3-amino-4- hydroxycyclopentyl)((R)-2- ((R)-1-(6-chloro-3′- (methylthio)biphenyl-2-yl)-1- hydroxy-5- methoxypentyl)morpholino) methanone |
| I-205a | 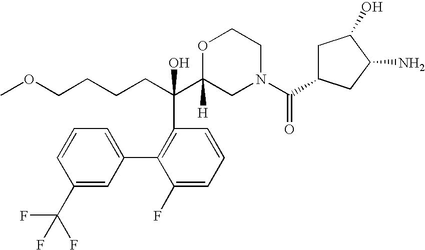 | ((1S,3R,4S)-3-amino-4- hydroxycyclopentyl)((R)-2- ((R)-1-(6-fluoro-3′- (trifluoromethyl)biphenyl-2- yl)-1-hydroxy-5- methoxypentyl)morpholino) methanone |
| I-205b | 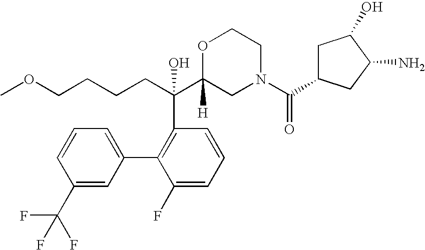 | ((1S,3R,4S)-3-amino-4- hydroxycyclopentyl)((R)-2- ((S)-1-(6-fluoro-3′- (trifluoromethyl)biphenyl-2- yl)-1-hydroxy-5- methoxypentyl)morpholino) methanone |
| I-206a | 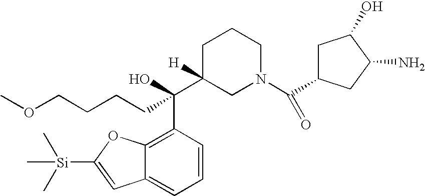 | ((1S,3R,4S)-3-amino-4- hydroxycyclopentyl)((R)-3- ((S)-1-hydroxy-5-methoxy-1- (2-(trimethylsilyl) benzofuran-7- yl)pentyl)piperidin-1- yl)methanone |
| I-207a | 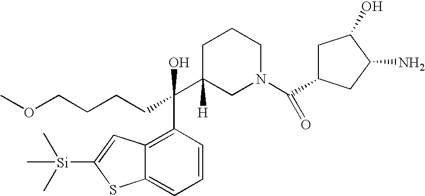 | ((1S,3R,4S)-3-amino-4- hydroxycyclopentyl)((R)-3- ((S)-1-hydroxy-5-methoxy-1- (2-(trimethylsilyl) benzo[b] thiophen-4-yl)pentyl)piperidin-1- yl)methanone |
| I-208a | 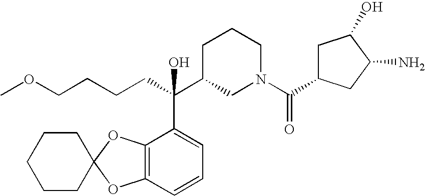 | ((1S,3R,4S)-3-amino-4- hydroxycyclopentyl)((R)-3- ((S)-1-hydroxy-5-methoxy-1- (spiro[benzo[d][1,3]dioxole- 2,1′-cyclohexane]-4- yl)pentyl)piperidin-1- yl)methanone |
| I-209a | 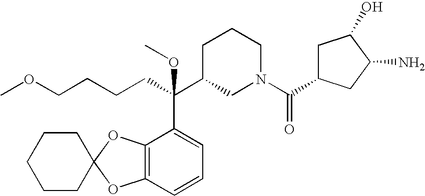 | ((1S,3R,4S)-3-amino-4- hydroxycyclopentyl)((R)-3- ((S)-1,5-dimethoxy-1- (spiro[benzo[d][1,3]dioxole- 2,1′-cyclohexane]-4- yl)pentyl)piperidin-1- yl)methanone |
| I-210a | 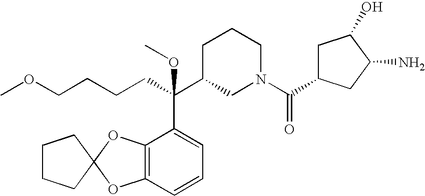 | ((1S,3R,4S)-3-amino-4- hydroxycyclopentyl)((R)-3- ((S)-1,5-dimethoxy-1- (spiro[benzo[d][1,3]dioxole- 2,1′-cyclopentane]-4- yl)pentyl)piperidin-1- yl)methanone |
| I-211a | 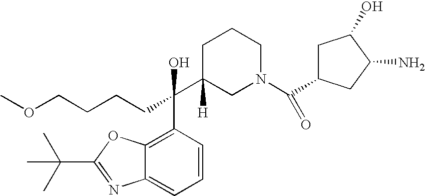 | ((1S,3R,4S)-3-amino-4- hydroxycyclopentyl)((R)-3- ((S)-1-(2-tert- butylbenzo[d]oxazol-7-yl)-1- hydroxy-5- methoxypentyl)piperidin-1- yl)methanone |
| I-212a | 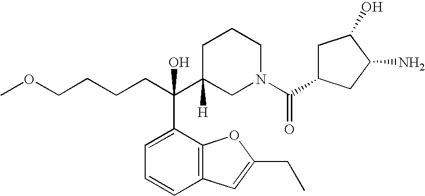 | ((1S,3R,4S)-3-amino-4- hydroxycyclopentyl)((R)-3- ((S)-1-(2-ethylbenzofuran-7- yl)-1-hydroxy-5- methoxypentyl)piperidin-1- yl)methanone |
| I-213a | 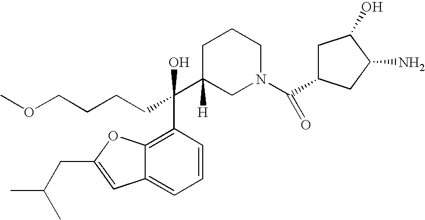 | ((1S,3R,4S)-3-amino-4- hydroxycyclopentyl)((R)-3- ((S)-1-hydroxy-1-(2- isobutylbenzofuran-7-yl)-5- methoxypentyl)piperidin-1- yl)methanone |
| I-214a | 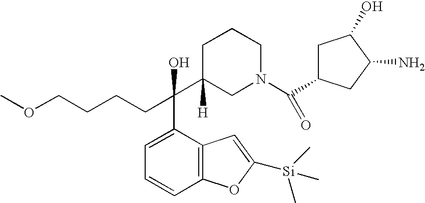 | ((1S,3R,4S)-3-amino-4- hydroxycyclopentyl)((R)-3- ((S)-1-hydroxy-5-methoxy-1- (2-(trimethylsilyl) benzofuran-4- yl)pentyl)piperidin-1- yl)methanone |
| I-215a | 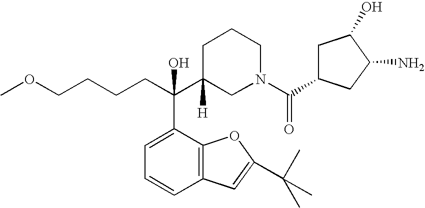 | ((1S,3R,4S)-3-amino-4- hydroxycyclopentyl)((R)-3- ((S)-1-(2-tert- butylbenzofyran-7-yl)-1- hydroxy-5- methoxypenxyl)piperidin-1- yl)methanone |
| I-216a | 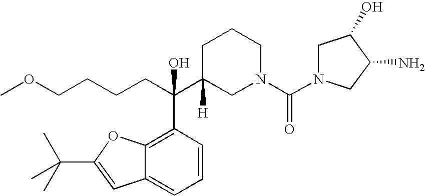 | ((3R,4S)-3-amino-4- hydroxypyrrolidin-1-yl)((R)- 3-((S)-1-(2-tert- butylbenzofuran-7-yl)-1- hydroxy-5- methoxypentyl)piperidin-1- yl)methanone |
| I-217a | 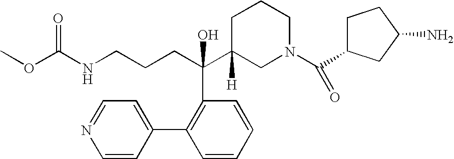 | methyl (S)-4-((R)-1- ((1R,3S)-3- aminocyclopentanecarbonyl) piperidin-3-yl)-4-hydroxy-4- (2-(pyridin-4- yl)phenyl)butylcarbamate |
| I-218a | 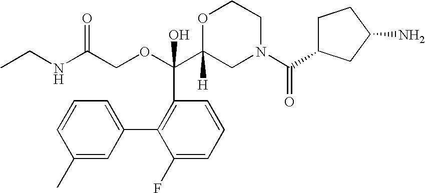 | 2-((S)-((R)-4-((1R,3S)-3- aminocyclopentanecarbonyl) morpholin-2-yl)(6-fluoro-3′- methylbiphenyl-2- yl)methoxy)-N- ethylacetamide |
| I-219a | 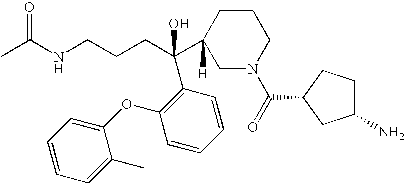 | N-((S)-4-((R)-1-((1R,3S)-3- aminocyclopentanecarbonyl) piperidin-3-yl)-4-hydroxy-4- (2-(o-tolyloxy)phenyl)butyl) acetamide |
| I-220a | 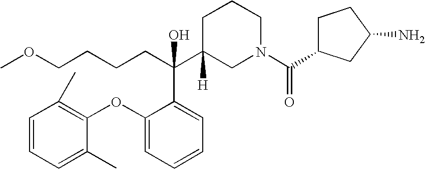 | ((1R,3S)-3- aminocyclopentyl)((R)-3- ((S)-1-(2-(2,6- dimethylphenoxy)phenyl)-1- hydroxy-5- methoxypentyl)piperidin-1- yl)methanone |
| I-221a | 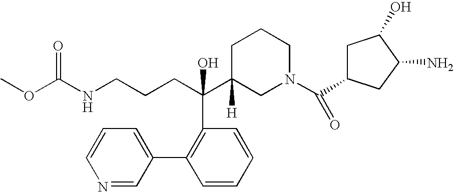 | methyl (S)-4-((R)-1- ((1S,3R,4S)-3-amino-4- hydroxycyclopentanecarbonyl) piperidin-3-yl)-4-hydroxy- 4-(2-(pyridin-3- yl)phenyl)butylcarbamate |
| I-222a | 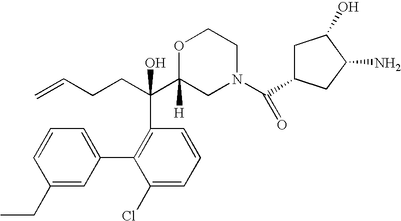 | ((1S,3R,4S)-3-amino-4- hydroxycyclopentyl)((R)-2- ((R)-1-(6-chloro-3′- ethylbiphenyl-2-yl)-1- hydroxypent-4- enyl)morpholino)methanone |
| I-223a | 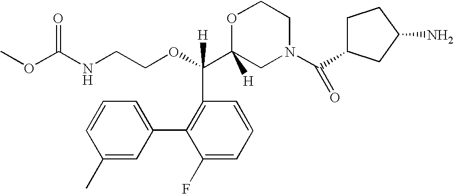 | methyl 2-((S)-((R)-4- ((1R,3S)-3- aminocyclopentanecarbonyl) morpholin-2-yl)(6-fluoro-3′- methylbiphenyl-2- yl)methoxy)ethylcarbamate |
| I-224a | 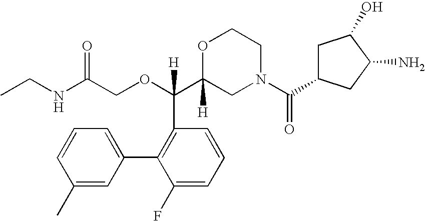 | 2-((S)-((R)-4-((1S,3R,4S)-3- amino-4- hydroxycyclopentanecarbonyl) morpholin-2-yl)(6-fluoro-3′- methylbiphenyl-2- yl)methoxy)-N- ethylacetamide |
| I-225a | 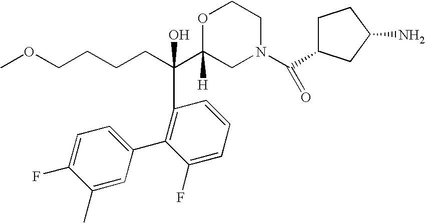 | ((1R,3S)-3- aminocyclopentyl)((R)-2- ((R)-1-(4′,6-difluoro-3′- methylbiphenyl-2-yl)-1- hydroxy-5- methoxypentyl)morpholino) methanone |
| I-226a | 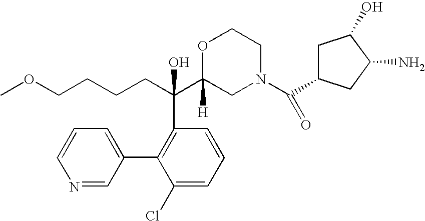 | ((1S,3R,4S)-3-amino-4- hydroxycyclopentyl)((R)-2- ((R)-1-(3-chloro-2-(pyridin- 3-yl)phenyl)-1-hydroxy-5- methoxypentyl)morpholino) methanone |
| I-226b | 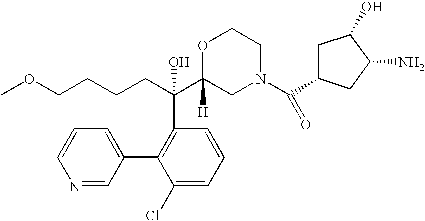 | ((1S,3R,4S)-3-amino-4- hydroxycyclopentyl)((R)-2- ((S)-1-(3-chloro-2-(pyridin-3- yl)phenyl)-1-hydroxy-5- methoxypentyl)morpholino) methanone |
| I-227a | 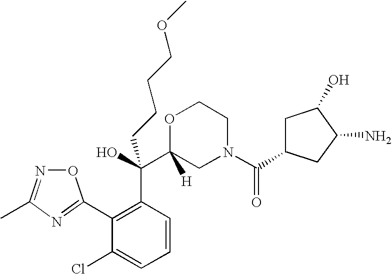 | ((1S,3R,4S)-3-amino-4- hydroxycyclopentyl)((R)-2- ((R)-1-(3-chloro-2-(3-methyl- 1,2,4-oxadiazol-5-yl)phenyl)- 1-hydroxy-5- methoxypentyl)morpholino) methanone |
| I-228a | 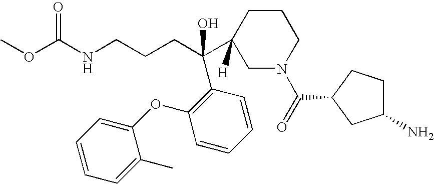 | methyl (S)-4-((R)-1-((1R,3S)- 3-aminocyclo- pentanecarbonyl) piperidin-3-yl)-4-hydroxy-4- (2-(o-tolyloxy)phenyl) butylcarbamate |
| I-229a | 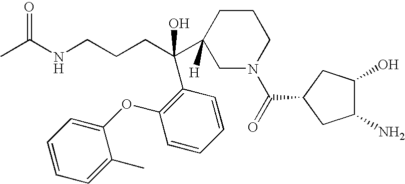 | N-((S)-4-((R)-1-((1S,3R,4S)- 3-amino-4- hydroxycyclopentanecarbonyl) piperidin-3-yl)-4-hydroxy- 4-(2-(o-tolyloxy)phenyl)butyl) acetamide |
| I-230a | 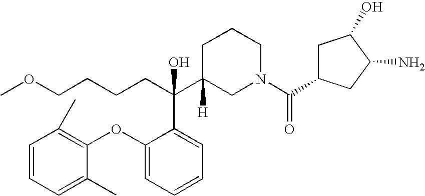 | ((1S,3R,4S)-3-amino-4- hydroxycyclopentyl)((R)-3- ((S)-1-(2-(2,6- dimethylphenoxy)phenyl)-1- hydroxy-5- methoxypentyl)piperidin-1- yl)methanone |
| I-231a | 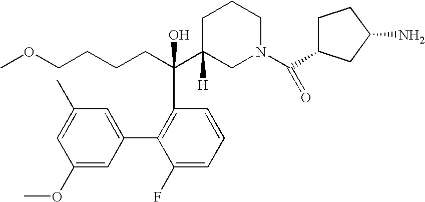 | ((1R,3S)-3- aminocyclopentyl)((R)-3- ((S)-1-(6-fluoro-3′-methoxy- 5′-methylbiphenyl-2-yl)-1- hydroxy-5- methoxypentyl)piperidin-1- yl)methanone |
| I-232a | 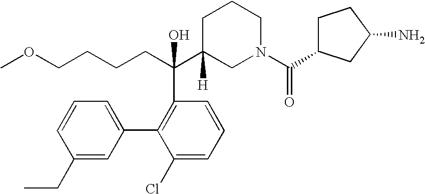 | ((1R,3S)-3- aminocyclopentyl)((R)-3- ((S)-1-(6-chloro-3′- ethylbiphenyl-2-yl)-1- hydroxy-5- methoxypentyl)piperidin-1- yl)methanone |
| I-233a | 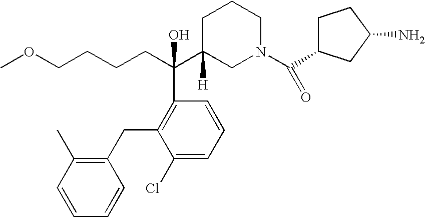 | ((1R,3S)-3- aminocyclopentyl)((R)-3- ((S)-1-(3-chloro-2-(2- methylbenzyl)phenyl)-1- hydroxy-5- methoxypentyl)piperidin-1- yl)methanone |
| I-234a | 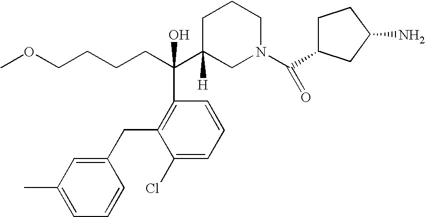 | ((1R,3S)-3- aminocyclopentyl)((R)-3- ((S)-1-(3-chloro-2-(3- methylbenzyl)phenyl)-1- hydroxy-5- methoxypentyl)piperidin-1- yl)methanone |
| I-235a | 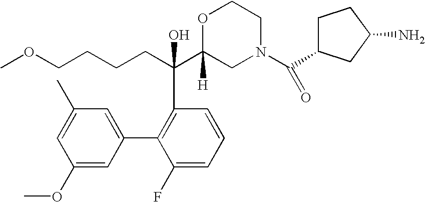 | ((1R,3S)-3- aminocyclopentyl)((R)-2- ((R)-2-(6-fluoro-3′-methoxy- 5′-methylbiphenyl-2-yl)-1- hydroxy-5- methoxypentyl)morpholino) methanone |
| I-236a | 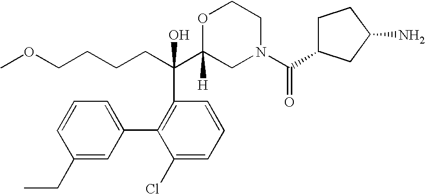 | ((1R,3S)-3- aminocyclopentyl)((R)-2- ((R)-1-(6-chloro-3′- ethylbiphenyl-2-yl)-1- hydroxy-5- methoxypentyl)morpholino) methanone |
| I-237a | 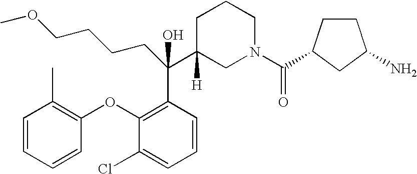 | ((1R,3S)-3- aminocyclopentyl)((R)-3- ((S)-1-(3-chloro-2-(o- tolyloxy)phenyl)-1-hydroxy- 5-methoxypentyl)piperidin-1- yl)methanone |
| I-238a | 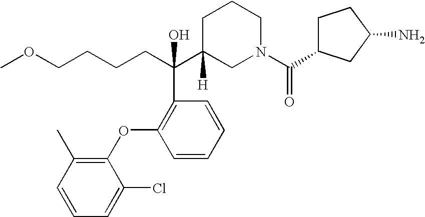 | ((1R,3S)-3- aminocyclopentyl)((R)-3- ((S)-1-(2-(2-chloro-6- methylphenoxy)phenyl)-1- hydroxy-5- methoxypentyl)piperidin-1- yl)methanone |
| I-239a | 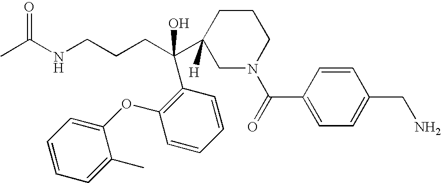 | N-((S)-4-((R)-1-(4- (aminomethyl)benzoyl) piperidin-3-yl)-4-hydroxy-4-(2- (o-tolyloxy)phenyl)butyl) acetamide |
| I-240a | 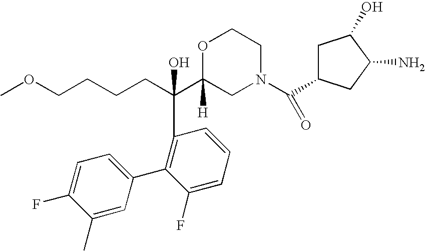 | ((1S,3R,4S)-3-amino-4- hydroxycyclopentyl)((R)-2- ((R)-1-(4′,6-difluoro-3′- methylbiphenyl-2-yl)-1- hydroxy-5- methoxypentyl)morpholino) methanone |
| I-240b | 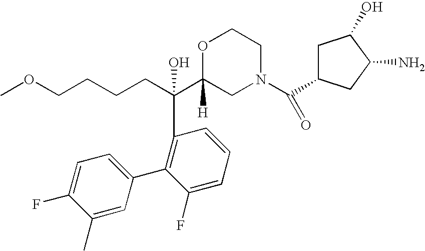 | ((1S,3R,4S)-3-amino-4- hydroxycyclopentyl)((R)-2- ((S)-1-(4′,6-difluoro-3′- methylbiphenyl-2-yl)-1- hydroxy-5- methoxypentyl)morpholino) methanone |
| I-241a | 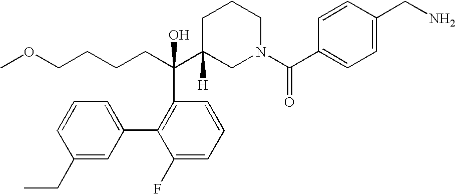 | (4-(aminomethyl)phenyl) ((R)-3-((S)-1-(3′-ethyl-6- fluorobiphenyl-2-yl)-1- hydroxy-5- methoxypentyl)piperidin-1- yl)methanone |
| I-242a | 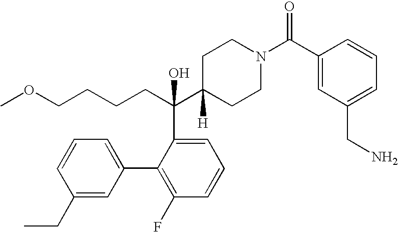 | (3-(aminomethyl)phenyl)(4- (1-(3′-ethyl-6-fluorobiphenyl- 2-yl)-1-hydroxy-5- methoxypentyl)piperidin-1- yl)methanone |
| I-243a | 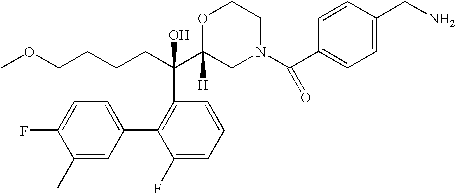 | (4-(aminomethyl)phenyl) ((R)-2-((R)-1-(4′,6-difluoro-3′- methylbiphenyl-2-yl)-1- hydroxy-5- methoxypentyl)morpholino) methanone |
| I-244a | 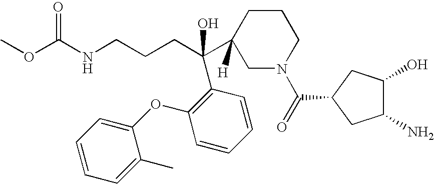 | methyl (S)-4-((R)-1- ((1S,3R,4S)-3-amino-4- hydroxycyclopentanecarbonyl) piperidin-3-yl)-4-hydroxy- 4-(2-(o-tolyloxy)phenyl) butylcarbamate |
| I-245a | 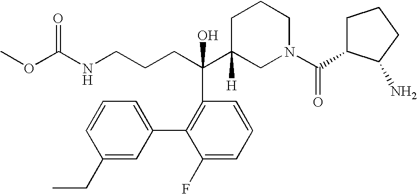 | methyl (S)-4-((R)-1-((1R,2S)- 2-aminocyclo- pentanecarbonyl) piperidin-3-yl)-4-(3′-ethyl-6- fluorobiphenyl-2-yl)-4- hydroxybutylcarbamate |
| I-246a | 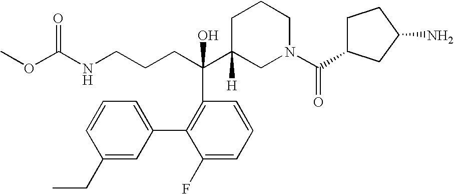 | methyl (S)-4-((R)-1-((1R,3S)- 3-aminocyclo- pentanecarbonyl) piperidin-3-yl)-4-(3′-ethyl-6- fluorobiphenyl-2-yl)-4- hydroxybutylcarbamate |
| I-247a | 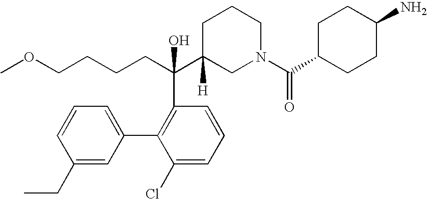 | (trans-4- aminocyclohexyl)((R)-3-((S)- 1-(6-chloro-3′-ethylbiphenyl- 2-yl)-1-hydroxy-5- methoxypentyl)piperidin-1- yl)methanone |
| I-248a | 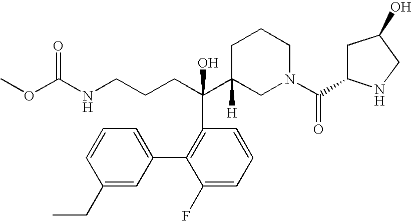 | methyl (S)-4-(3′-ethyl-6- fluorobiphenyl-2-yl)-4- hydroxy-4-((R)-1-((2S,4R)-4- hydroxypyrrolidine-2- carbonyl)piperidin-3- yl)butylcarbamate |
| I-249a | 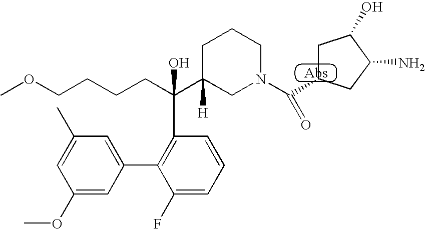 | ((1S,3R,4S)-3-amino-4- hydroxycyclopentyl)((R)-3- ((S)-1-(6-fluoro-3′-methoxy- 5′-methylbiphenyl-2-yl)-1- hydroxy-5- methoxypentyl)piperidin-1- yl)methanone |
| I-250a | 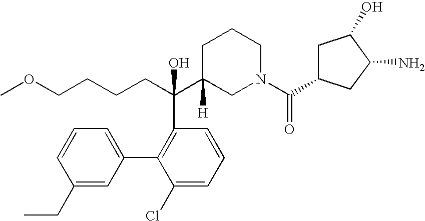 | ((1S,3R,4S)-3-amino-4- hydroxycyclopentyl)((R)-3- ((S)-1-(6-chloro-3′- ethylbiphenyl-2-yl)-1- hydroxy-5- methoxypentyl)piperidin-1- yl)methanone |
| I-251a | 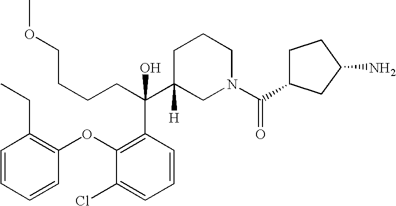 | ((1R,3S)-3- aminocyclopentyl)((R)-3- ((S)-1-(3-chloro-2-(2- ethylphenoxy)phenyl)-1- hydroxy-5- methoxypentyl)piperidin-1- yl)methanone |
| I-252a | 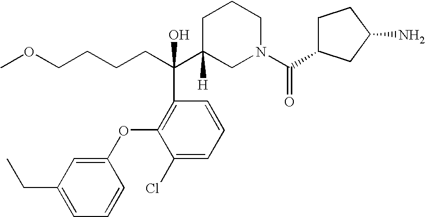 | ((1R,3S)-3- aminocyclopentyl)((R)-3- ((S)-1-(3-chloro-2-(3- ethylphenoxy)phenyl)-1- hydroxy-5- methoxypentyl)piperidin-1- yl)methanone |
| I-253a | 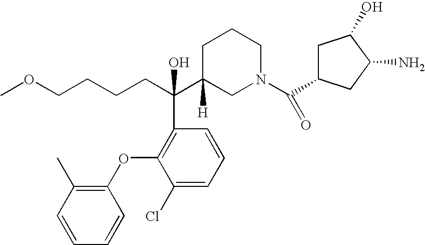 | ((1S,3R,4S)-3-amino-4- hydroxycyclopentyl)((R)-3- ((S)-1-(3-chloro-2-(2- methylbenzyl)phenyl)-1- hydroxy-5- methoxypentyl)piperidin-1- yl)methanone |
| I-254a | 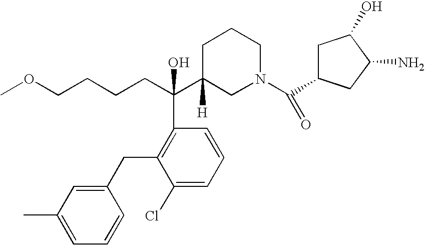 | ((1S,3R,4S)-3-amino-4- hydroxycyclopentyl)((R)-3- ((S)-1-(3-chloro-2-(3- methylbenzyl)phenyl)-1- hydroxy-5- methoxypentyl)piperidin-1- yl)methanone |
| I-255a | 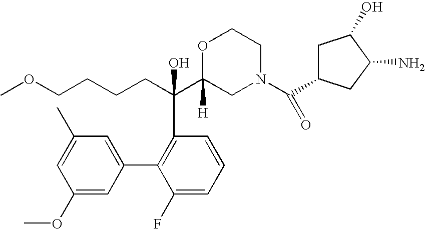 | ((1S,3R,4S)-3-amino-4- hydroxycyclopentyl)((R)-2- ((R)-1-(6-fluoro-3′-methoxy- 5′-methylbiphenyl-2-yl)-1- hydroxy-5- methoxypentyl)morpholino) methanone |
| I-256a | 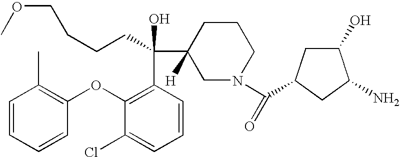 | ((1S,3R,4S)-3-amino-4- hydroxycyclopentyl)((R)-3- ((S)-1-(3-chloro-2-(o- tolyloxy)phenyl)-1-hydroxy- 5-methoxypentyl)piperidin-1- yl)methanone |
| I-257a | 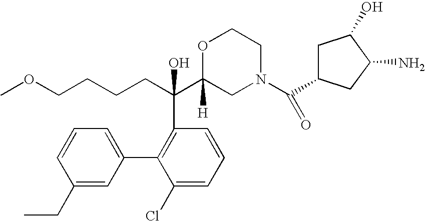 | ((1S,3R,4S)-3-amino-4- hydroxycyclopentyl)((R)-2- ((R)-1-(6-chloro-3′- ethylbiphenyl-2-yl)-1- hydroxy-5- methoxypentyl)morpholino) methanone |
| I-258a | 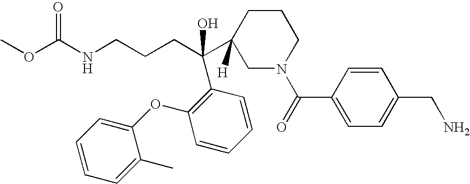 | methyl (S)-4-((R)-1-(4- (aminomethyl)benzoyl) piperidin-3-yl)-4-hydroxy- 4-(2-(o-tolyloxy)phenyl) butylcarbamate |
| I-259a | 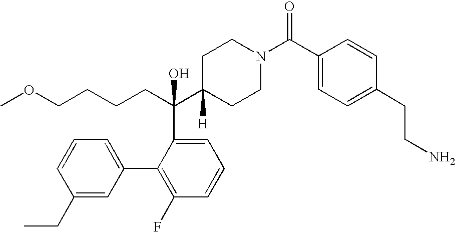 | (4-(2-aminoethyl)phenyl)(4- (1-(3′-ethyl-6-fluorobiphenyl- 2-yl)-1-hydroxy-5- methoxypentyl)piperidin-1- yl)methanone |
| I-260a | 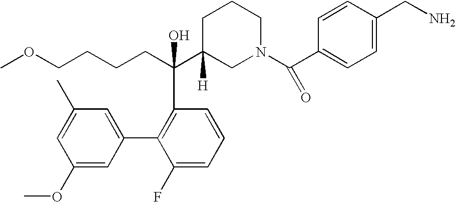 | (4-(aminomethyl)phenyl) ((R)-3-((S)-1-(6-fluoro-3′- methoxy-5′-methylbiphenyl- 2-yl)-1-hydroxy-5- methoxypentyl)piperidin-1- yl)methanone |
| I-261a | 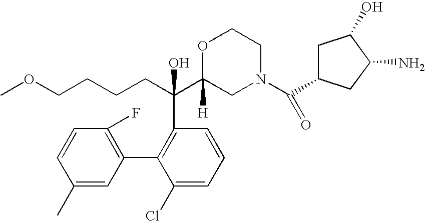 | ((1S,3R,4S)-3-amino-4- hydroxycyclopentyl)((2R)-2- ((1R)-1-(6-chloro-2′-fluoro- 5′-methylbiphenyl-2-yl)-1- hydroxy-5- methoxypentyl)morpholino) methanone |
| I-262a | 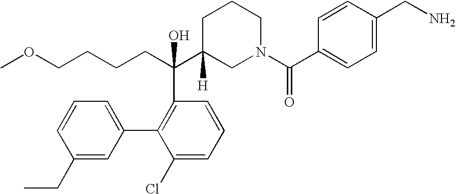 | (4-(aminomethyl)phenyl) ((R)-3-((S)-1-(6-chloro-3′- ethylbiphenyl-2-yl)-1- hydroxy-5- methoxypenlyl)piperidin-1- yl)methanone |
| I-263a | 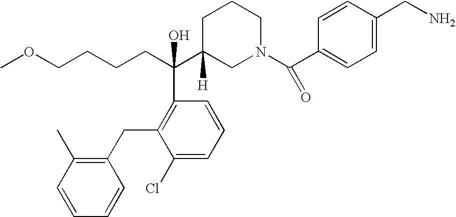 | (4-(aminomethyl)phenyl) ((R)-3-((S)-1-(3-chloro-2-(2- methylbenzyl)phenyl)-1- hydroxy-5- methoxypentyl)piperidin-1- yl)methanone |
| I-264a | 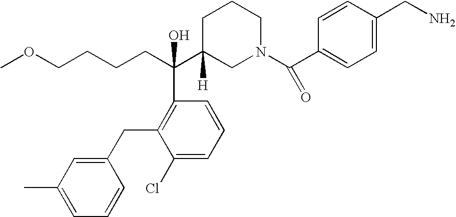 | (4-(aminomethyl)phenyl) ((R)-3-((S)-1-(3-chloro-2-(3- methylbenzyl)phenyl)-1- hydroxy-5- methoxypentyl)piperidin-1- yl)methanone |
| I-265a | 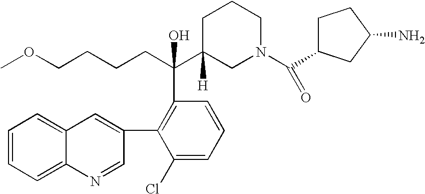 | ((1R,3S)-3- aminocyclopentyl)((R)-3- ((S)-1-(3-chloro-2-(quinolin- 3-yl)phenyl)-1-hydroxy-5- methoxypentyl)piperidin-1- yl)methanone |
| I-265b | 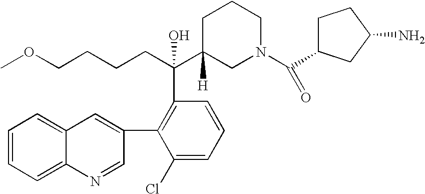 | ((1R,3S)-3- aminocyclopentyl)((R)-3- ((R)-1-(3-chloro-2-(quinolin- 3-yl)phenyl)-1-hydroxy-5- methoxypentyl)piperidin-1- yl)methanone |
| I-266a | 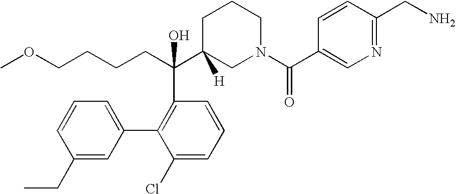 | (6-(aminomethyl)pyridin-3- yl)((R)-3-((S)-1-(6-chloro-3′- ethylbiphenyl-2-yl)-1- hydroxy-5- methoxypentyl)piperidin-1- yl)methanone |
| I-267a | 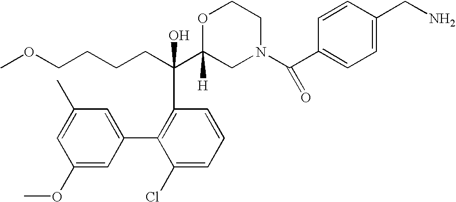 | (4-(aminomethyl)phenyl) ((R)-2-((R)-1-(6-fluoro- 3′-methoxy- 5′-methylbiphenyl-2-yl)-1- hydroxy-5- methoxypentyl)morpholino) methanone |
| I-268a | 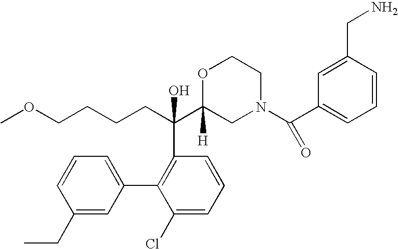 | (3-(aminomethyl)phenyl) ((R)-2-((R)-1-(6-chloro-3′- ethylbiphenyl-2-yl)-1- hydroxy-5- methoxypentyl)morpholino) methanone |
| I-269a | 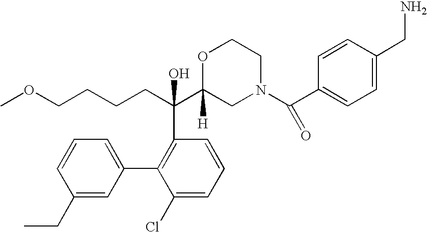 | (4-(aminomethyl)phenyl) ((R)-2-((R)-1-(6-chloro-3′- ethylbiphenyl-2-yl)-1- hydroxy-5- methoxypentyl)morpholino) methanone |
| I-270a | 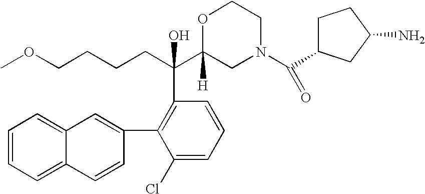 | ((1R,3S)-3- aminocyctopentyl)((R)-2- ((R)-1-(3-chloro-2- (naphthalen-2-yl)phenyl)-1- hydroxy-5- methoxypentyl)morpholino) methanone |
| I-271a | 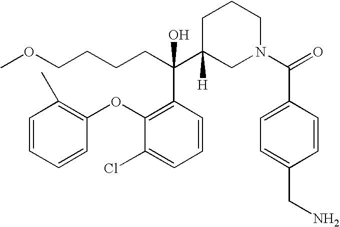 | (4-(aminomethyl)phenyl) ((R)-3-((S)-1-(3-chloro-2-(o- tolyloxy)phenyl)-1-hydroxy- methoxypentyl)piperidin-1- yl)methanone |
| I-272a | 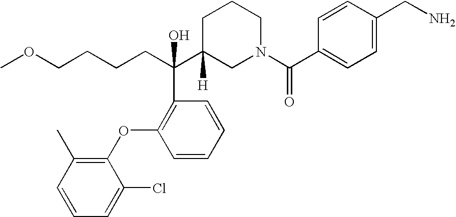 | (4-(aminomethyl)phenyl) ((R)-3-((S)-1-(2-(2-chloro-6- methylphenoxy)phenyl)-1- hydroxy-5- methoxypentyl)piperidin-1- yl)methanone |
| I-273a | 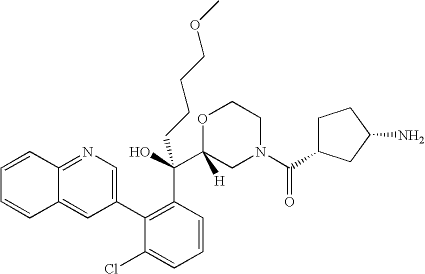 | ((1R,3S)-3- aminocyclopentyl)((R)-2- ((R)-1-(3-chloro-2-(quinolin- 3-yl)phenyl)-1-hydroxy-5- methoxypentyl)morpholino) methanone |
| I-274a | 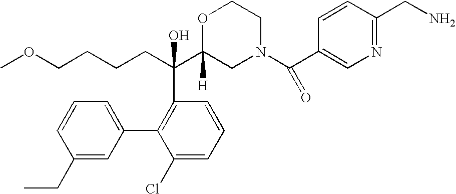 | (6-(aminomethyl)pyridin-3- yl)((R)-2-((R)-1-(6-chloro-3′- ethylbiphenyl-2-yl)-1- hydroxy-5- methoxypentyl)morpholino) methanone |
| I-275a | 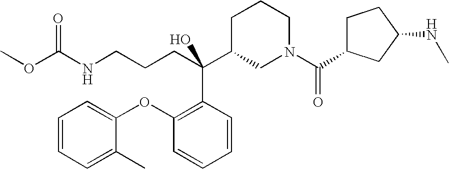 | methyl ((4S)-4-hydroxy-4- ((3R)-1-{[(1R,3S)-3- (methylamino)cyclopentyl] carbonyl}-3-piperidinyl)-4-{2- [(2-methylphenyl)oxy]- phenyl}butyl)carbamate |
| I-276a | 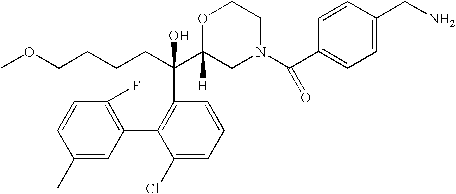 | (4-(aminomethyl)phenyl) ((2R)-2-((1R)-1-(6-chloro- 2′-fluoro-5′-methylbiphenyl- 2-yl)-1-hydroxy-5- methoxypentyl)morpholino) methanone |
| I-277a | 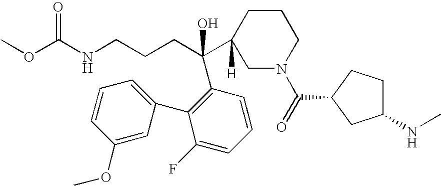 | methyl (S)-4-(6-fluoro-3′- methoxybiphenyl-2-yl)-4- hydroxy-4-((R)-1-((1R,3S)-3- (methylamino)cyclopentane- carbonyl)piperidin-3- yl)butylcarbamate |
| I-278a | 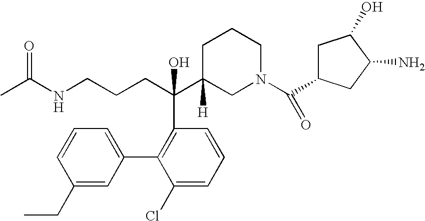 | N-((S)-4-((R)-1-((1S,3R,4S)- 3-amino-4- hydroxycyclopentanecarbonyl) piperidin-3-yl)4-(6-chloro- 3′-ethylbiphenyl-2-yl)-4- hydroxybutyl)acetamide |
| I-279a | 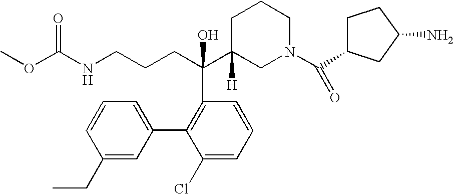 | methyl (S)-4-((R)-1-((1R,3S)- 3-aminocyclo- pentanecarbonyl) piperidin-3-yl)-4-(6-chloro- 3′-ethylbiphenyl-2-yl)-4- hydroxybutylcarbamate |
| I-280a | 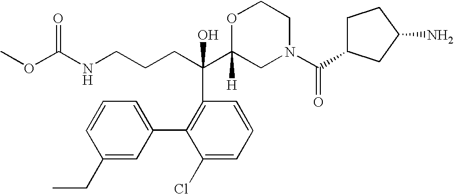 | methyl (R)-4-((R)-4- ((1R,3S)-3- aminocyclopentanecarbonyl) morpholin-2-yl)-4-(6-chloro- 3′-ethylbiphenyl-2-yl)-4- hydroxybutylcarbamate |
| I-281a | 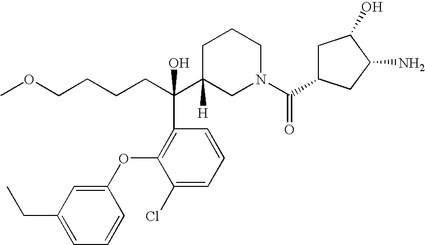 | ((1S,3R,4S)-3-amino-4- hydroxycyclopentyl)((R)-3- ((S)-1-(3-chloro-2-(3- ethylphenoxy)phenyl)-1- hydroxy-5- methoxypentyl)piperidin-1- yl)methanone |
| I-282a | 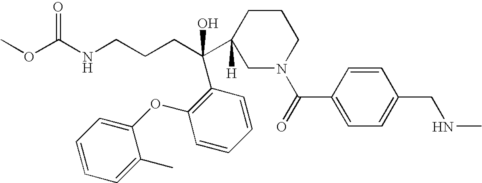 | methyl (S)-4-hydroxy-4-((R)- 1-(4-((methylamino)methyl) benzoyl)piperidin-3-yl)-4-(2- (o-tolyloxy)phenyl) butylcarbamate |
| I-283a | 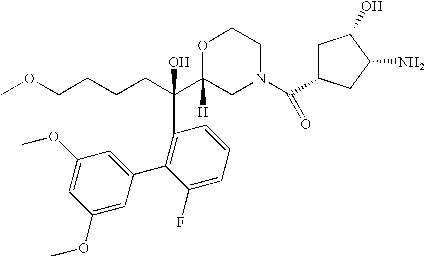 | ((1S,3R,4S)-3-amino-4- hydroxycyclopentyl)((R)-2- ((R)-1-(6-fluoro-3′,5′- dimethoxybiphenyl-2-yl)-1- hydroxy-5- methoxypentyl)morpholino) methanone |
| I-284a | 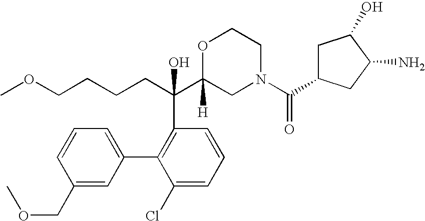 | ((1S,3R,4S)-3-amino-4- hydroxycyclopentyl)((R)-2- ((R)-1-(6-chloro-3′- (methoxymethyl)biphenyl-2- yl)-1-hydroxy-5- methoxypentyl)morpholino) methanone |
| I-285a | 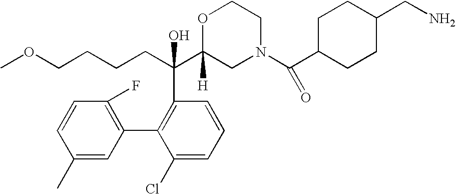 | (4-(aminomethyl)cyclohexyl) ((2R)-2-((1R)-1-(6-chloro-2′- fluoro-5′-methylbiphenyl-2- yl)-1-hydroxy-5- methoxypentyl)morpholino) methanone |
| I-286a | 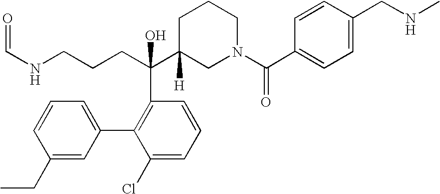 | N-((S)-4-(6-chloro-3′- ethylbiphenyl-2-yl)-4- hydroxy-4-((R)-1-(4- ((methylamino)methyl) benzoyl)piperidin-3- yl)butyl)formamide |
| I-287a | 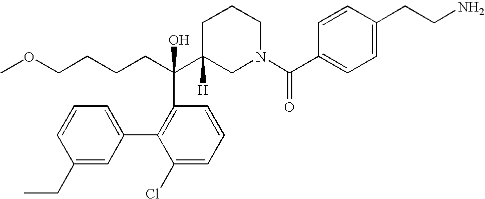 | ((R)-3-((S)-1-(3-chloro-2-(2- methylbenzyl)phenyl)-1- hydroxy-5- methoxypentyl)piperidin-1- yl)(4-((methylamino)methyl) phenyl)methanone |
| I-288a | 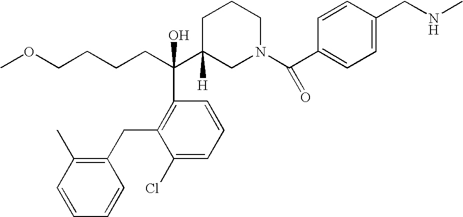 | ((R)-3-((S)-1-(3-chloro-2-(2- methylbenzyl)phenyl)-1- hydroxy-5- methoxypentyl)piperidin-1- yl)(4-((methylamino)methyl) phenyl)methanone |
| I-289a | 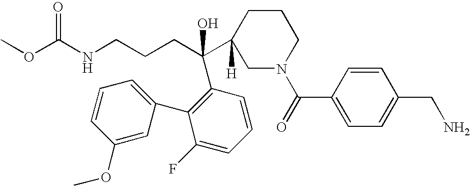 | methyl (S)-4-((R)-1-(4- (aminomethyl)benzoyl) piperidin-3-yl)-4-(6-fluoro-3′- methoxybiphenyl-2-yl)-4- hydroxybutylcarbamate |
| I-290a | 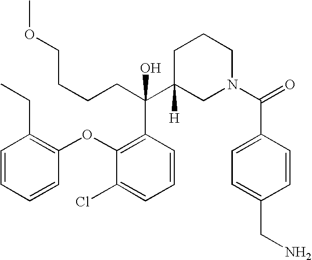 | (4-(aminomethyl)phenyl) ((R)-3-((S)-1-(3-chloro-2-(2- ethylphenoxy)phenyl)-1- hydroxy-5- methoxypentyl)piperidin-1- yl)methanone |
| I-291a | 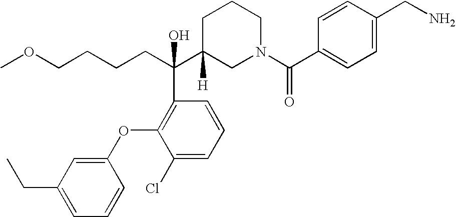 | (4-(aminomethyl)phenyl) ((R)-3-((S)-1-(3-chloro-2-(3- ethylphenoxy)phenyl)-1- hydroxy-5- methoxypentyl)piperidin-1- yl)methanone |
| I-292a | 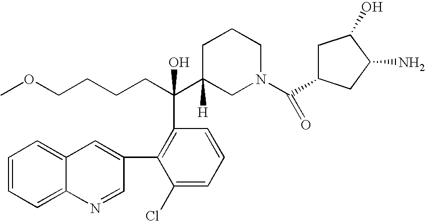 | ((1S,3R,4S)-3-amino-4- hydroxycyclopentyl)((R)-3- ((S)-1-(3-chloro-2-(quinolin- 3-yl)phenyl)-1-hydroxy-5- methoxypentyl)piperidin-1- yl)methanone |
| I-292b | 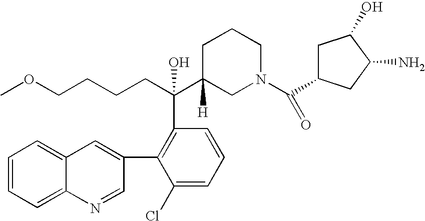 | ((1S,3R,4S)-3-amino-4- hydroxycyclopentyl)((R)-3- ((S)-1-(3-chloro-2-(quinolin- 3-yl)phenyl)-1-hydroxy-5- methoxypentyl)piperidin-1- yl)methanone |
| I-293a | 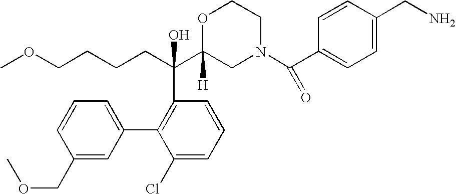 | (4-(aminomethyl)phenyl) ((R)-2-((R)-1-(6-chloro-3′- (methoxymethyl)biphenyl-2- yl)-1-hydroxy-5- methoxypentyl)morpholino) methanone |
| I-294a | 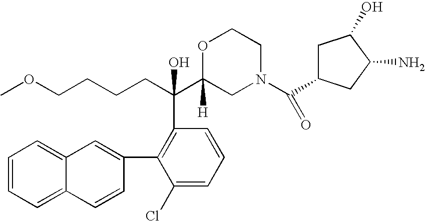 | ((1S,3R,4S)-3-amino-4- hydroxycyclopentyl)((R)-2- ((R)-1-(3-chloro-2- (naphthalen-2-yl)phenyl)-1- hydroxy-5- methoxypentyl)morpholino) methanone |
| I-295a | 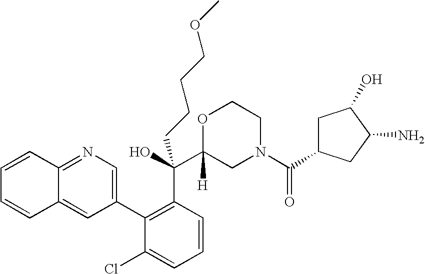 | ((1S,3R,4S)-3-amino-4- hydroxycyclopentyl)((R)-2- ((R)-1-(3-chloro-2-(quinolin- 3-yl)phenyl)-1-hydroxy-5- methoxypentyl)morpholino) methanone |
| I-295b | 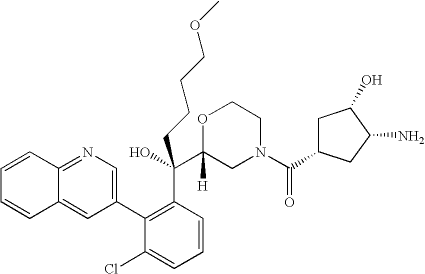 | ((1S,3R,4S)-3-amino-4- hydroxycyclopentyl)((R)-2- ((S)-1-(3-chloro-2-(quinolin- 3-yl)phenyl)-1-hydroxy-5- methoxypentyl)morpholino) methanone |
| I-296a | 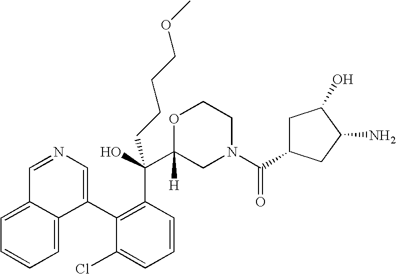 | ((1S,3R,4S)-3-amino-4- hydroxycyclopentyl)((2R)-2- ((1R)-1-(3-chloro-2- (isoquinolin-4-yl)phenyl)-1- hydroxy-5- methoxypentyl)morpholino) methanone |
| I-296b | 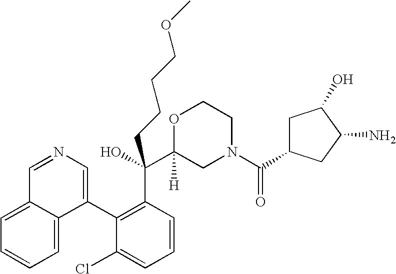 | ((1S,3R,4S)-3-amino-4- hydroxycyclopentyl)((2S)-2- ((1R)-1-(3-chloro-2- (isoquinolin-4-yl)phenyl)-1- hydroxy-5- methoxypentyl)morpholino) methanone |
| I-297a | 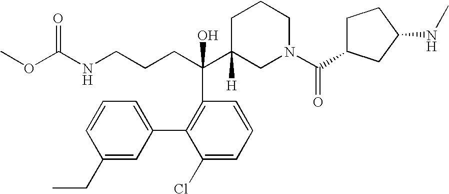 | methyl (S)-4-(6-chloro-3′- ethylbiphenyl-2-yl)-4- hydroxy-4-((R)-1-((1R,3S)-3- (methylamino)cyclopentane- carbonyl)piperidin-3- yl)butylcarbamate |
| I-298a | 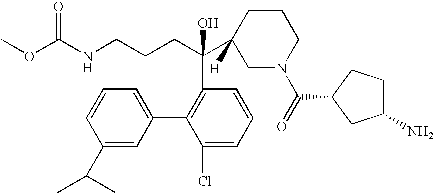 | methyl (S)-4-((R)-1-((1R,3S)- 3-aminocyclo- pentanecarbonyl) piperidin-3-yl)-4-(6-chloro- 3′-isopropylbiphenyl-2-yl)-4- hydroxybutylcarbamate |
| I-299a | 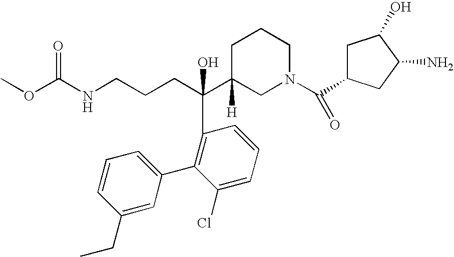 | methyl (S)-4-((R)-1- ((1S,3R,4S)-3-amino-4- hydroxycyclopentanecarbonyl) piperidin-3-yl)-4-(6-chloro- 3′-ethylbiphenyl-2-yl)-4- hydroxybutylcarbamate |
| I-300a | 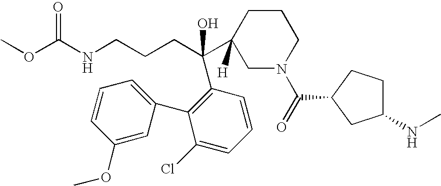 | methyl (S)-4-(6-chloro-3′- methoxybiphenyl-2-yl)-4- hydroxy-4-((R)-1-((1R,3S)-3- (methylamino)cyclopentane- carbonyl)piperidin-3- yl)butylcarbamate |
| I-301a | 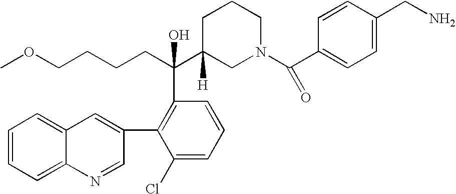 | (4-(aminomethyl)phenyl) ((R)-3-((S)-1-(3-chloro-2- (quinolin-3-yl)phenyl)-1- hydroxy-5- methoxypentyl)piperidin-1- yl)methanone |
| I-302a | 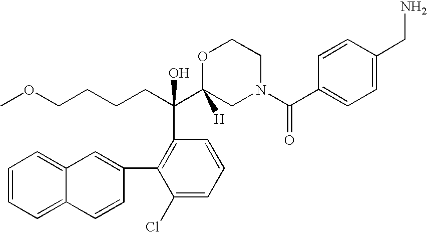 | (4-(aminometbyl)phenyl) ((R)-2-((R)-1-(3-chloro-2- (naphthalen-2-yl)phenyl)-1- hydroxy-5- methoxypentyl)morpholino) methanone |
| I-303a | 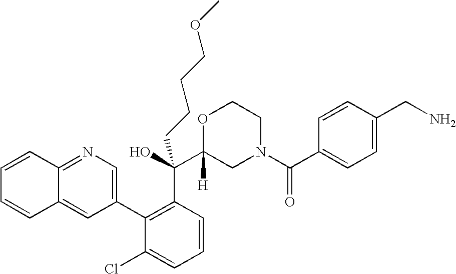 | (4-(aminomethyl)phenyl) ((R)-2-((R)-1-(3-chloro-2- (quinolin-3-yl)phenyl)-1- hydroxy-5- methoxypentyl)morpholino) methanone |
| I-303b | 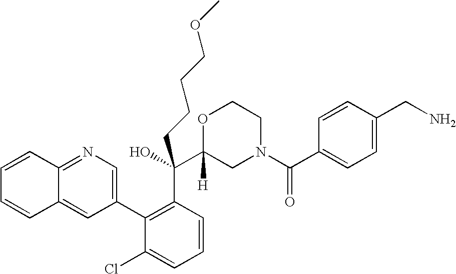 | (4-(aminomethyl)phenyl) ((R)-2-((S)-1-(3-chloro-2- (quinolin-3-yl)phenyl)-1- hydroxy-5- methoxypentyl)morpholino) methanone |
| I-304a | 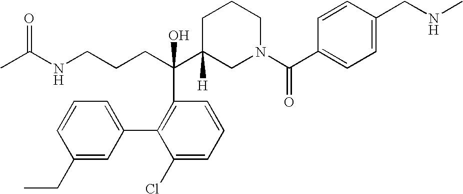 | N-((S)-4-(6-chloro-3′- ethylbiphenyl-2-yl)-4- hydroxy-4-((R)-1-(4- ((methylamino)methyl) benzoyl)piperidin-3- yl)butyl)acetamide |
| I-305a | 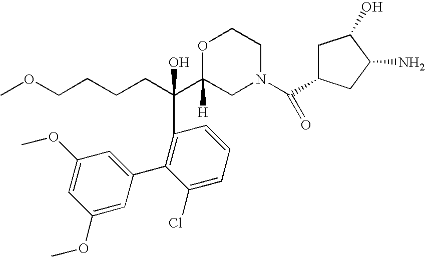 | ((1S,3R,4S)-3-amino-4- hydroxycyclopentyl)((R)-2- ((R)-1-(6-chloro-3′,5′- dimethoxybiphenyl-2-yl)-1- hydroxy-5- methoxypentyl)morpholino) methanone |
| I-306a | 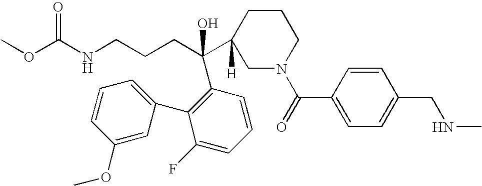 | methyl (S)-4-(6-fluoro-3′- methoxybiphenyl-2-yl)-4- hydroxy-4-((R)-1-(4- ((methylamino)methyl) benzoyl)piperidin-3- yl)butylcarbamate |
| I-307a | 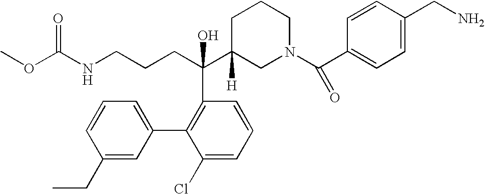 | methyl (S)-4-((R)-1-(4- (aminomethyl)benzoyl) piperidin-3-yl)-4-(6-chloro-3′- ethylbiphenyl-2-yl)-4- hydroxybutylcarbamate |
| I-307b | 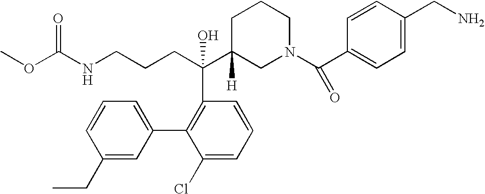 | methyl (R)-4-((R)-1-(4- (aminomethyl)benzoyl) piperidin-3-yl)-4-(6-chloro-3′- ethylbiphenyl-2-yl)-4- hydroxybutylcarbamate |
| I-308a | 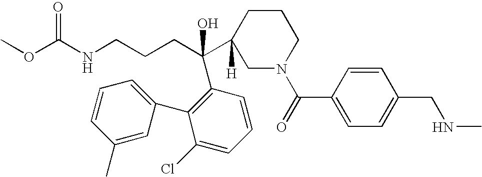 | methyl (S)-4-(6-chloro-3′- methylbiphenyl-2-yl)-4- hydroxy-4-((R)-1-(4- ((methylamino)methyl) benzoyl)piperidin-3- yl)butylcarbamate |
| I-309a | 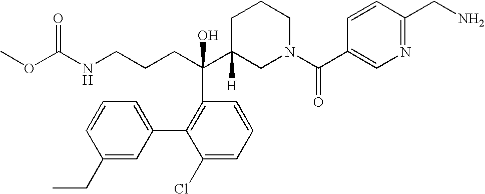 | methyl (S)-4-((R)-1-(6- (aminomethyl)nicotinoyl) piperidin-3-yl)-4-(6-chloro-3′- ethylbiphenyl-2-yl)-4- hydroxybutylcarbamate |
| I-310a | 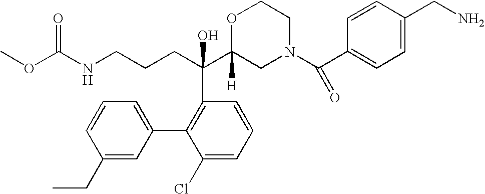 | methyl (R)-4-((R)-4-(4- (aminomethyl)benzoyl) morpholin-2-yl)- 4-(6-chloro-3′- ethylbiphenyl-2-yl)-4- hydroxybutylcarbamate |
| I-311a | 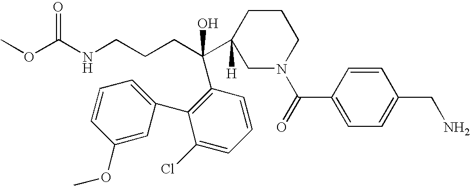 | methyl (S)-4-((R)-1-(4- (aminomethyl)benzoyl) piperidin-3-yl)-4-(6-chloro-3′- methoxybiphenyl-2-yl)-4- hydroxybutylcarbamate |
| I-312a | 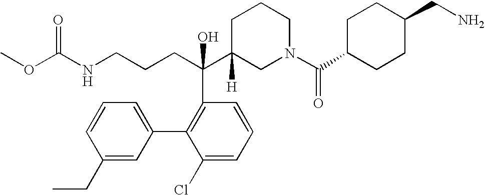 | methyl (S)-4-((R)-1-(trans-4- (aminomethyl)cyclohexane- carbonyl)piperidin-3-yl)-4-(6- chloro-3′-ethylbiphenyl-2-yl)- 4-hydroxybutylcarbamate |
| I-313a | 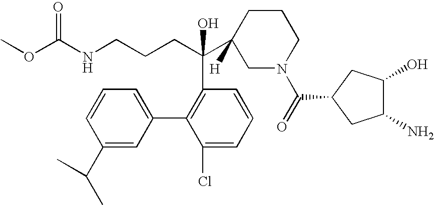 | methyl (S)-4-((R)-1- ((1S,3R,4S)-3-amino-4- hydroxycyclopentanecarbonyl) piperidin-3-yl)-4-(6-chloro- 3′-isopropylbiphenyl-2-yl)-4- hydroxybutylcarbamate |
| I-314a | 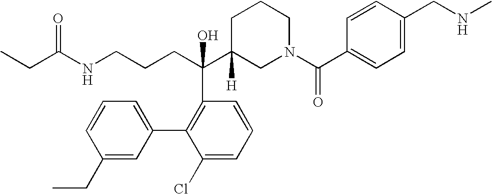 | N-((S)-4-(6-chloro-3′- ethylbiphenyl-2-yl)-4- hydroxy-4-((R)-1-(4- ((methylamino)methyl) benzoyl)piperidin-3- yl)butyl)propionamide |
| I-315a | 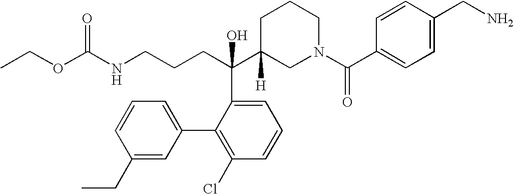 | ethyl (S)-4-((R)-1-(4- (aminomethyl)benzoyl) piperidin-3-yl)-4-(6-chloro-3′- ethylbiphenyl-2-yl)-4- hydroxybutylcarbamate |
| I-316a | 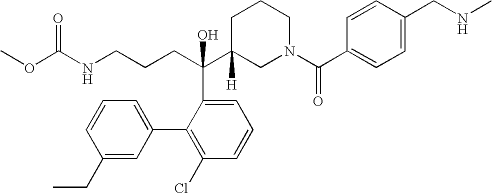 | methyl (S)-4-(6-chloro-3′- ethylbiphenyl-2-yl)-4- hydroxy-4-((R)-1-(4- ((methylamino)methyl) benzoyl)piperidin-3- yl)butylcarbamate |
| I-317a | 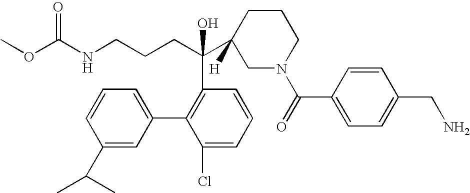 | methyl (S)-4-((R)-1-(4- (aminomethyl)benzoyl) piperidin-3-yl)-4-(6-chloro-3′- isopropylbiphenyl-2-yl)-4- hydroxybutylcarbamate |
| I-318a | 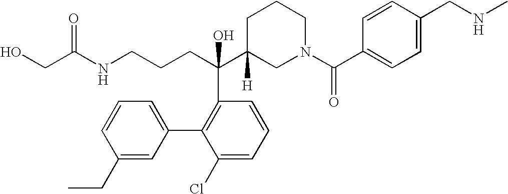 | N-((S)-4-(6-chloro-3′- ethylbiphenyl-2-yl)-4- hydroxy-4-((R)-1-(4- ((methylamino)methyl) benzoyl)piperidin-3-yl)butyl)- 2-hydroxyacetamide |
| I-319a | 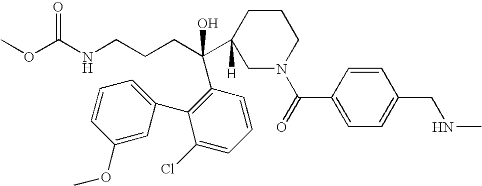 | methyl (S)-4-(6-chloro-3′- methoxybipbenyl-2-yl)-4- hydroxy-4-((R)-1-(4- ((methylamino)methyl) benzoyl)piperidin-3- yl)butylcarbamate |
| I-320a | 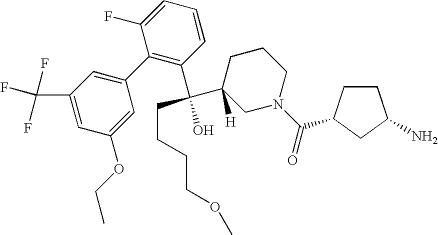 | ((1R,3S)-3- aminocyclopentyl)((R)-3- ((S)-1-(3′-ethoxy-6-fluoro-5′- (trifluoromethyl)biphenyl-2- yl)-1-hydroxy-5- methoxypentyl)piperidin-1- yl)methanone |
| I-321a | 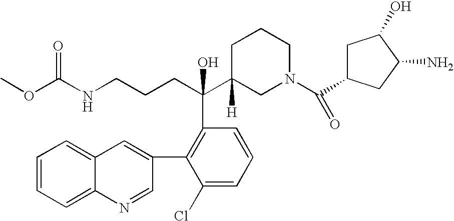 | methyl (S)-4-((R)-1- ((1S,3R,4S)-3-amino-4- hydroxycyclopentanecarbonyl) piperidin-3-yl)-4-(3-chloro- 2-(quinolin-3-yl)phenyl)-4- hydroxybutylcarbamate |
| I-322a | 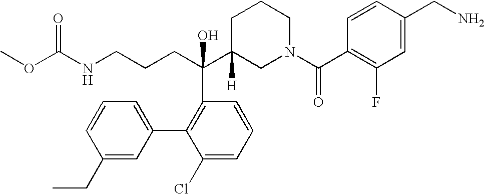 | methyl (S)-4-((R)-1-(4- (aminomethyl)-2- fluorobenzoyl)piperidin-3- yl)-4-(6-chloro-3′- ethylbiphenyl-2-yl)-4- hydroxybutylcarbamate |
| I-323a | 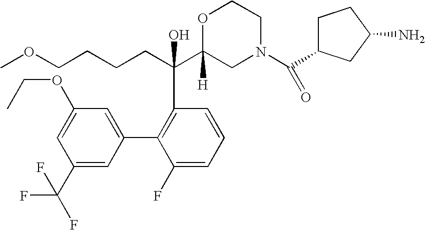 | ((1R,3S)-3- aminocyclopentyl)((R)-2- ((R)-1-(3′-ethoxy-6-fluoro-5′- (trifluoromethyl)biphenyl-2- yl)-1-hydroxy-5- methoxypentyl)morpholino) methanone |
| I-324a | 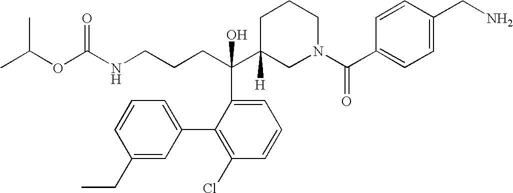 | isopropyl (S)-4-((R)-1-(4- (aminomethyl)benzoyl) piperidin-3-yl)-4-(6-chloro-3′- ethylbiphenyl-2-yl)-4- hydroxybutylcarbamate |
| I-325a | 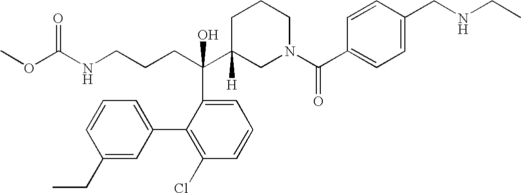 | methyl (S)-4-(6-chloro-3′- ethylbiphenyl-2-yl)-4-((R)-1- (4-((ethylamino)methyl) benzoyl)piperidin-3-yl)-4- hydroxybutylcarbamate |
| I-326a | 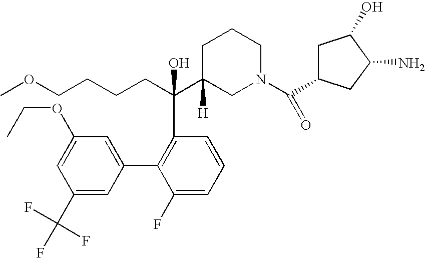 | ((1S,3R,45)-3-amino-4- hydroxycyclopentyl)((R)-3- ((S)-1-(3′-ethoxy-6-fluoro-5′- (trifluoromethyl)biphenyl-2- yl)-1-hydroxy-5- methoxypentyl)piperidin-1- yl)methanone |
| I-326b | 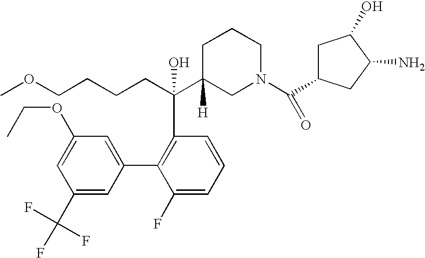 | ((1S,3R,4S)-3-amino-4- hydroxycyclopentyl)((R)-3- ((R)-1-(3′-ethoxy-6-fluoro-5′- (trifluoromethyl)biphenyl-2- yl)-1-hydroxy-5- methoxypentyl)piperidin-1- yl)methanone |
| I-327a | 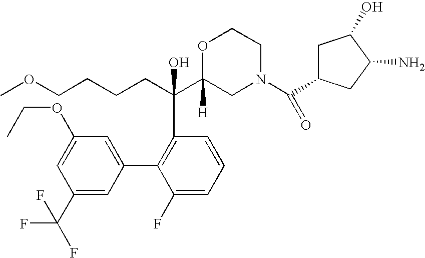 | ((1S,3R,4S)-3-amino-4- hydroxycyclopentyl)((R)-2- ((R)-1-(3′-ethoxy-6-fluoro-5′- (trifluoromethyl)biphenyl-2- yl)-1-hydroxy-5- methoxypentyl)morpholino) methanone |
| I-328a | 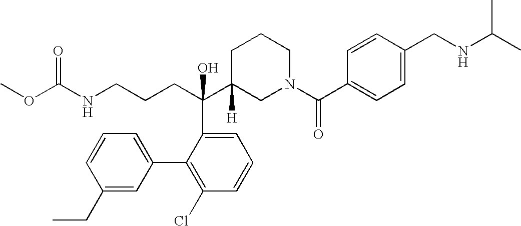 | methyl (S)-4-(6-chloro-3′- ethylbiphenyl-2-yl)-4- hydroxy-4-((R)-1-(4- ((isopropylamino)methyl) benzoyl)piperidin-3- yl)butylcarbamate |
| I-329a | 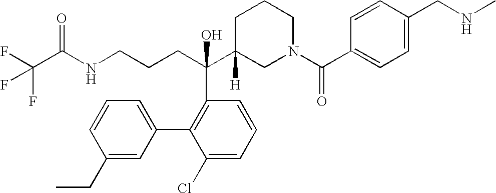 | N-((S)-4-(6-chloro-3′- ethylbiphenyl-2-yl)-4- hydroxy-4-((R)-1-(4- ((methylamino)methyl) benzoyl)piperidin-3-yl)butyl)- 2,2,2-trifluoroacetamide |
| I-330a | 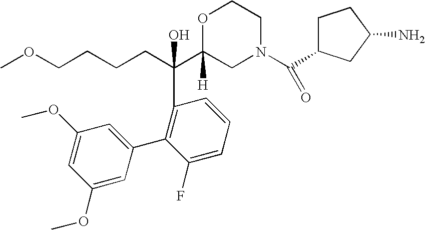 | ((1R,3S)-3- aminocyclopentyl)((R)-2- ((R)-1-(6-fluoro-3′,5′- dimethoxybiphenyl-2-yl)-1- hydroxy-5- methoxypentyl)morpholino) methanone |
| I-331a | 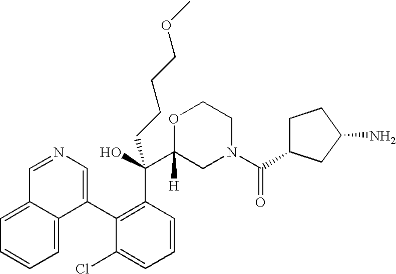 | ((1R,3S)-3- aminocyclopentyl)((2R)-2- ((1R)-1-(3-chloro-2- (isoquinolin-4-yl)phenyl)-1- hydroxy-5- methoxypentyl)morpholino) methanone |
| I-332a | 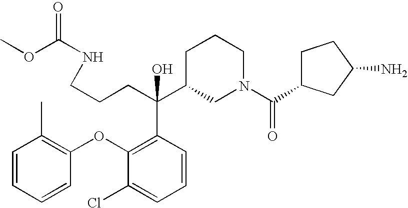 | methyl (4-((3R)-1-{[(1R,3S)- 3-aminocyclopentyl] carbonyl}-3- piperidinyl)-4-{3-chloro-2-[(2- methylphenyl)oxy]phenyl}-4- hydroxybutyt)carbamate |
| I-333a | 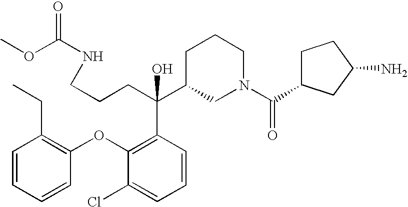 | methyl (4-((3R)-1-{[(1R,3S)- 3-aminocyclopentyl] carbonyl}-3- piperidinyl)-4-{3-chloro-2- [(2-ethylphenyl)oxy]phenyl}- 4-hydroxybutyl)carbamate |
| I-334a | 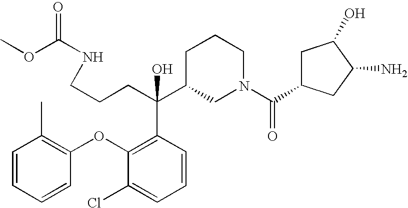 | methyl (4-((3R)-1- {[(1S,3R,4S)-3-amino-4- hydroxycyclopentyl]carbonyl}- 3-piperidinyl)-4-{3-chloro-2- [(2-methylphenyl)oxy]phenyl}- 4-hydroxybutyl)carbamate |
| I-335a | 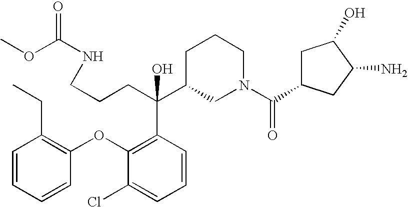 | methyl (4-((3R)-1- {[(1S,3R,4S)-3-amino-4- hydroxycyclopentyl]carbonyl}- 3-piperidinyl)-4-{3-chloro-2- [(2-ethylphenyl)oxy]phenyl}- 4-hydroxybutyl)carbamate |
| I-336a | 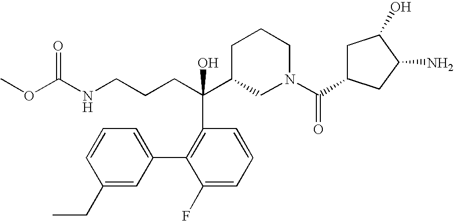 | methyl [4-((3R)-1- {[(1S,3R,4S)-3-amino-4- hydroxycyclopentyl]carbonyl}- 3-piperidinyl)-4-(3′-ethyl-6- fluoro-2-biphenylyl)-4- hydroxybutyl]carbamate |
| I-337a | 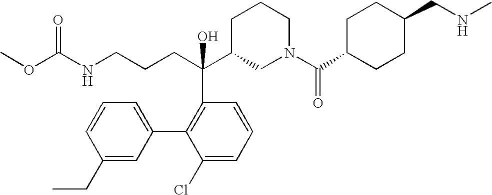 | methyl {4-(6-chloro-3′-ethyl- 2-biphenylyl)-4-hydroxy-4- [(3R)-1-({trans-4- [(methylamino)methyl] cyclohexyl}carbonyl)-3- piperidinyl]bulyl}carbamate |
| I-338a | 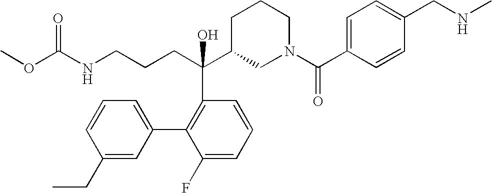 | methyl {4-(3′-ethyl-6-fluoro- 2-biphenylyl)-4-hydroxy-4- [(3R)-1-({4- [(methylamino)methyl] phenyl}carbonyl)-3- piperidinyl]butyl}carbamate |
| I-339a | 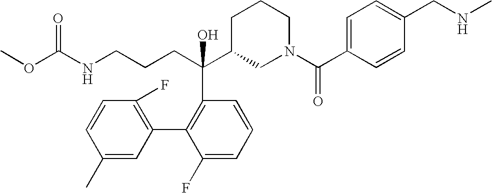 | methyl {4-(2′,6-difluoro-5′- methyl-2-biphenylyl)-4- hydroxy-4-[(3R)-1-({4- [(methylamino)methyl] phenyl}carbonyl)-3- piperidinyl]butyl}carbamate |
| I-340a | 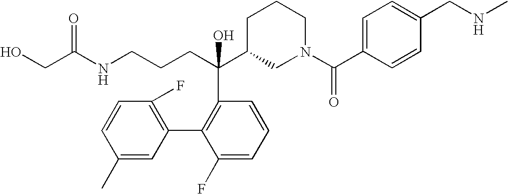 | N-{4-(2′,6-difluoro-5′-methyl- 2-biphenylyl)-4-hydroxy-4- [(3R)-1-({4- [(methylamino)methyl] phenyl}carbonyl)-3- piperidinyl]butyl}-2- hydroxyacetamide |

or salts thereof.

The following are preferred compounds of Formula I: I-5a, I-8a, I-9a, I-18a, I-19a, I-20a, I-22a, I-25a, I-25b, I-29a, I-30a, I-37b, I-38a, I-39a, I-40a, I-41a, I-41b, I-43a, I-45a, I-47a, I-47b, I-49a, I-51a, I-53a, I-54a, I-58a, I-59a, I-60a, I-61a, I-63a, I-64a, I-66a, I-67a, I-68a, I-69a, I-70a, I-71a, I-73b, I-74a, I-74b, I-76a, I-77a, I-79a, I-84a, I-87a, I-89a, I-90a, I-91a, I-92a, I-93a, I-94a, I-95a, I-96a, I-101a, I-102a, I-105a, I-108a, I-109a, I-111a, I-113a, I-115a, I-117a, I-118c, I-120a, I-122a, I-125a, I-126a, I-127a, I-128a, I-129a, I-129b, I-130a, I-131a, I-132a, I-135a, I-136a, I-137a, I-139a, I-140a, I-141a, I-143a, I-144a, I-145a, I-146a, I-148a, I-149a, I-150a, I-151a, I-152a, I-153a, I-154a, I-155a, I-156a, I-157a, I-158a, I-159a, I-161a, I-162a, I-163a, I-164a, I-165a, I-165b, I-166a, I-167a, I-168a, I-169a, I-170a, I-171a, I-172a, I-173a, I-175a, I-176a, I-177a, I-186a, I-187a, I-189a, I-191a, I-192a, I-193a, I-193b, I-194a, I-195a, I-196a, I-197a, I-200a, I-201a, I-201b, I-202a, I-203a I-204a, I-205a, I-205b, I-206a, I-213a, I-215a, I-216a, I-219a, I-222a, I-223a, I-228a, I-229a, I-231a, I-236a, I-237a, I-240a, I-244a, I-246a, I-249a, I-250a, I-251a, I-252a, I-253a, I-255a, I-256a, I-257a, I-258a, I-261a, I-262a, I-265a, I-270a, I-275a, I-277a, I-278a, I-279a, I-280a, I-281a, I-282a, I-283a, I-284a, I-286a, I-289a, I-292a, I-294a, I-295a, I-295b, I-295c, I-296a, I-297a, I-298a, I-299a, I-300a, I-304a, I-305a, I-306a, I-307a, I-307b, I-308a, I-309a, I-310a, I-311a, I-312a, I-313a, I-314a, I-316a, I-317a, I-318a, I-319a, I-321a, I-322a, I-325a, I-328a, I-329a or the salts thereof.

The following are more preferred compounds of Formula I: I-41a, I-59a, I-66a, I-67a, I-70a, I-71a, I-95a, I-122a, I-126a, I-129a, I-130a, I-131a, I-135a, I-140a, I-141a, I-145a, I-146a, I-149a, I-150a, I-151a, I-152a, I-153a, I-154a, I-155a, I-156a, I-158a, I-159a, I-161a, I-163a, I-164a, I-165a, I-166a, I-167a, I-170a, I-172a, I-175a, I-176a, I-177a, I-191a, I-192a, I-196a, I-197a, I-201a, I-201b, I-202a, I-203a, I-204a, I-205a, I-229a, I-236a, I-237a, I-244a, I-246a, I-250a, I-251a, I-256a, I-257a, I-275a, I-277a, I-278a, I-279a, I-280a, I-281a, I-294a, I-297a, I-298a, I-299a, I-300a, I-304a, I-306a, I-307a, I-308a, I-309a, I-310a, I-311a, I-312a, I-313a, I-314a, I-316a, I-317a, I-318a, I-319a, I-321a, I-322a, I-325a, I-328a, I-329a, or the salts thereof.

The following are highly preferred compounds of Formula I: I-141a, I-145a, I-163a, I-164a, I-167a, I-175a, I-196a, I-244a, I-246a, I-257a, I-257a, I-278a, I-279a, I-280a, I-297a, I-299a, I-304a, I-310a, I-312a, I-313a, I-314a, I-316a, I-318a, I-329a, I-332a, I-333a, I-334a, I-335a, I-336a, I-337a, I-338a, I-339a, I-340a or the salts thereof.

When any variable (e.g., aryl, heterocyclyl, R¹, R², etc.) occurs more than once in a compound, its definition on each occurrence is independent of any other occurrence.

“Alkyl” means a saturated aliphatic branched or straight-chain mono- or di-valent hydrocarbon radical having the specified number of carbon atoms. Thus, “(C₁-C₈)alkyl” means a radical having from 1-8 carbon atoms in a linear or branched arrangement. “(C₁-C₆)alkyl” includes methyl, ethyl, propyl, butyl, pentyl, and hexyl.

“Cycloalkyl” means a saturated aliphatic cyclic hydrocarbon radical having the specified number of carbon atoms. Thus, (C₃-C₇)cycloalkyl means a radical having from 3-8 carbon atoms arranged in a ring. (C₃-C₇)cycloalkyl includes cyclopropyl, cyclobutyl, cyclopentyl, cyclohexyl, and cycloheptyl.

Haloalkyl and halocycloalkyl include mono, poly, and perhaloalkyl groups where the halogens are independently selected from fluorine, chlorine, and bromine.

Saturated heterocyclic rings are 4-, 5-, 6-, and 7-membered heterocyclic rings containing 1 to 4 heteroatoms independently selected from N, O, and S, and include pyrrolidine, piperidine, tetrahydrofuran, tetrahydropyran, tetrahydrothiophene, tetrahydrothiopyran, isoxazolidine, 1,3-dioxolane, 1,3-dithiolane, 1,3-dioxane, 1,4-dioxane, 1,3-dithiane, 1,4-dithiane, morpholine, thiomorpholine, thiomorpholine 1,1-dioxide, tetrahydro-2H-1,2-thiazine 1,1-dioxide, and isothiazolidine 1,1-dioxide. Oxo substituted saturated heterocyclic rings include tetrahydrothiophene 1-oxide, tetrahydrothiophene 1,1-dioxide, thiomorpholine 1-oxide, thiomorpholine 1,1-dioxide, tetrahydro-2H-1,2-thiazine 1,1-dioxide, and isothiazolidine 1,1-dioxide, pyrrolidin-2-one, piperidin-2-one, piperazin-2-one, and morpholin-2-one.

“Heteroaryl” means a monovalent heteroaromatic monocyclic and polycyclic ring radical. Heteroaryl rings are 5- and 6-membered aromatic heterocyclic rings containing 1 to 4 heteroatoms independently selected from N, O, and S, and include furan, thiophene, pyrrole, imidazole, pyrazole, oxazole, isoxazole, thiazole, isothiazole, 1,2,3-triazole, 1,2,4-triazole, 1,3,4-oxadiazole, 1,2,5-thiadiazole, 1,2,5-thiadiazole 1-oxide, 1,2,5-thiadiazole 1,1-dioxide, 1,3,4-thiadiazole, pyridine, pyridine-N-oxide, pyrazine, pyrimidine, pyridazine, 1,2,4-triazine, 1,3,5-triazine, and tetrazole. Bicyclic heteroaryl rings are bicyclo[4.4.0] and bicyclo[4,3.0] fused ring systems containing 1 to 4 heteroatoms independently selected from N, O, and S, and include indolizine, indole, isoindole, benzo[b]furan, benzo[b]thiophene, indazole, benzimidazole, benzthiazole, purine, 4H-quinolizine, quinoline, isoquinoline, cinnoline, phthalazine, quinazoline, quinoxaline, 1,8-naphthyridine, and pteridine.

“Alkoxy” means an alkyl radical attached through an oxygen linking atom. “(C₁-C₄)alkoxy” includes methoxy, ethoxy, propoxy, and butoxy.

“Aromatic” means an unsaturated cycloalkyl ring system.

“Aryl” means an aromatic monocyclic, or polycyclic ring system. Aryl systems include phenyl, naphthalenyl, fluorenyl, indenyl, azulenyl, and anthracenyl.

“Hetero” refers to the replacement of at least one carbon atom member in a ring system with at least one heteroatom selected from N, S, and O. A hetero ring may have 1, 2, 3, or 4 carbon atom members replaced by a heteroatom.

“Unsaturated ring” means a ring containing one or more double bonds and include cyclopentene, cyclohexene, cyclopheptene, cyclohexadiene, benzene, pyrroline, pyrazole, 4,5-dihydro-1H-imidazole, imidazole, 1,2,3,4-tetrahydropyridine, 1,2,3,6-tetrahydropyridine, pyridine and pyrimidine.

### Enantiomers, Diastereomers, and Salts

Certain compounds of Formula I may exist in various stereoisomeric or tautomeric forms. The invention encompasses all such forms, including active compounds in the form of essentially pure enantiomers, racemic mixtures, and tautomers, including forms those not depicted structurally.

The compounds of the invention may be present in the form of pharmaceutically acceptable salts. For use in medicines, the salts of the compounds of the invention refer to non-toxic “pharmaceutically acceptable salts.” Pharmaceutically acceptable salt forms include pharmaceutically acceptable acidic/anionic or basic/cationic salts.

Pharmaceutically acceptable acidic/anionic salts include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

The compounds of the invention include pharmaceutically acceptable anionic salt forms, wherein the anionic salts include the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, mandelate, mesylate, methylsulfate, mucate, napsylate, nitrate, pamoate, pantothenate, phosphate/diphosphate, polygalacturonate, salicylate, stearate, subacetate, succinate, sulfate, tannate, tartrate, teoclate, tosylate, and triethiodide salts.

The anionic salt form of a compound of the invention includes the acetate, bromide, camsylate, chloride, edisylate, fumarate, hydrobromide, hydrochloride, iodide, isethionate, lactate, mesylate, maleate, napsylate, salicylate, sulfate, and tosylate salts.

When a disclosed compound or its pharmaceutically acceptable salt is named or depicted by structure, it is to be understood that solvates or hydrates of the compound or its pharmaceutically acceptable salts are also included. “Solvates” refer to crystalline forms wherein solvent molecules are incorporated into the crystal lattice during crystallization. Solvate may include water or nonaqueous solvents such as ethanol, isopropanol, DMSO, acetic acid, ethanolamine, and EtOAc. Solvates, wherein water is the solvent molecule incorporated into the crystal lattice, are typically referred to as “hydrates.” Hydrates include stoichiometric hydrates as well as compositions containing variable amounts of water.

When a disclosed compound or its pharmaceutically acceptable salt is named or depicted by structure, it is to be understood that the compound, including solvates thereof, may exist in crystalline forms, non-crystalline forms or a mixture thereof. The compound or its pharmaceutically acceptable salts or solvates may also exhibit polymorphism (i.e. the capacity to occur in different crystalline forms). These different crystalline forms are typically known as “polymorphs.” It is to be understood that when named or depicted by structure, the disclosed compound and its pharmaceutically acceptable salts, solvates or hydrates also include all polymorphs thereof. Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in solidifying the compound. For example, changes in temperature, pressure, or solvent may result in different polymorphs. In addition, one polymorph may spontaneously convert to another polymorph under certain conditions.

It may be necessary and/or desirable during synthesis to protect sensitive or reactive groups on any of the molecules concerned. Representative conventional protecting groups are described in T. W. Greene and P. G. M. Wuts “Protective Groups in Organic Synthesis” John Wiley & Sons, Inc., New York 1999. Protecting groups may be added and removed using methods well known in the art.

The invention also includes various isomers and mixtures thereof “Isomer” refers to compounds that have the same composition and molecular weight but differ in physical and/or chemical properties. The structural difference may be in constitution (geometric isomers) or in the ability to rotate the plane of polarized light (stereoisomers).

Certain of the disclosed aspartic protease inhibitors may exist in various stereoisomeric forms. Stereoisomers are compounds which differ only in their spatial arrangement. Enantiomers are pairs of stereoisomers whose mirror images are not superimposable, most commonly because they contain an asymmetrically substituted carbon atom that acts as a chiral center. “Enantiomer” means one of a pair of molecules that are mirror images of each other and are not superimposable. Diastereomers are stereoisomers that are not related as mirror images, most commonly because they contain two or more asymmetrically substituted carbon atoms. The symbol “*” in a structural formula represents the presence of a chiral carbon center. “R” and “S” represent the configuration of substituents around one or more chiral carbon atoms. Thus, “R*” and “S*” denote the relative configurations of substituents around one or more chiral carbon atoms. When a chiral center is not defined as R or S, a mixture of both configurations is present.

“Racemate” or “racemic mixture” means a compound of equimolar quantities of two enantiomers, wherein such mixtures exhibit no optical activity; i.e., they do not rotate the plane of polarized light.

“Geometric isomer” means isomers that differ in the orientation of substituent atoms in relationship to a carbon-carbon double bond, to a cycloalkyl ring, or to a bridged bicyclic system.

Atoms (other than H) on each side of a carbon-carbon double bond may be in an E (substituents are on opposite sides of the carbon-carbon double bond) or Z (substituents are oriented on the same side) configuration.

Atoms (other than H) attached to a carbocyclic ring may be in a cis or trans configuration. In the “cis” configuration, the substituents are on the same side in relationship to the plane of the ring; in the “trans” configuration, the substituents are on opposite sides in relationship to the plane of the ring. A mixture of “cis” and “trans” species is designated “cis/trans”.

The point at which a group or moiety is attached to the remainder of the compound or another group or moiety can be indicated by [symbol] which represents [symbol] or “_{———}”.

“R,” “S,” “S*,” “R*,” “E,” “Z,” “cis,” and “trans,” indicate configurations relative to the core molecule.

The compounds of the invention may be prepared as individual isomers by either isomer-specific synthesis or resolved from an isomeric mixture. Conventional resolution techniques include forming the salt of a free base of each isomer of an isomeric pair using an optically active acid (followed by fractional crystallization and regeneration of the free base), forming the salt of the acid form of each isomer of an isomeric pair using an optically active amine (followed by fractional crystallization and regeneration of the free acid), forming an ester or amide of each of the isomers of an isomeric pair using an optically pure acid, amine or alcohol (followed by chromatographic separation and removal of the chiral auxiliary), or resolving an isomeric mixture of either a starting material or a final product using various well known chromatographic methods.

When the stereochemistry of a disclosed compound is named or depicted by structure, the named or depicted stereoisomer is at least 60%, 70%, 80%, 90%, 99% or 99.9% by weight pure relative to the other stereoisomers. When a single enantiomer is named or depicted by structure, the depicted or named enantiomer is at least 60%, 70%, 80%, 90%, 99% or 99.9% by weight optically pure. Percent optical purity by weight is the ratio of the weight of the enantiomer over the weight of the enantiomer plus the weight of its optical isomer.

When a disclosed compound is named or depicted by structure without indicating the stereochemistry, and the inhibitor has at least one chiral center, it is to be understood that the name or structure encompasses one enantiomer of inhibitor free from the corresponding optical isomer, a racemic mixture of the inhibitor and mixtures enriched in one enantiomer relative to its corresponding optical isomer.

When a disclosed aspartic protease inhibitor is named or depicted by structure without indicating the stereochemistry and has at least two chiral centers, it is to be understood that the name or structure encompasses a diastereomer free of other diastereomers, a pair of diastereomers free from other diastereomeric pairs, mixtures of diastereomers, mixtures of diastereomeric pairs, mixtures of diastereomers in which one diastereomer is enriched relative to the other diastereomer(s) and mixtures of diastereomeric pairs in which one diastereomeric pair is enriched relative to the other diastereomeric pair(s).

The compounds of the invention are useful for ameliorating or treating disorders or diseases in which decreasing the levels of aspartic protease products is effective in treating the disease state or in treating infections in which the infectious agent depends upon the activity of an aspartic protease. In hypertension elevated levels of angiotensin I, the product of renin catalyzed cleavage of angiotensinogen are present. Thus, the compounds of the invention can be used in the treatment of hypertension, heart failure such as (acute and chronic) congestive heart failure; left ventricular dysfunction; cardiac hypertrophy; cardiac fibrosis; cardiomyopathy (e.g., diabetic cardiac myopathy and post-infarction cardiac myopathy); supraventricular and ventricular arrhythmias; arial fibrillation; atrial flutter; detrimental vascular remodeling; myocardial infarction and its sequelae; atherosclerosis; angina (whether unstable or stable); renal failure conditions, such as diabetic nephropathy; glomerulonephritis; renal fibrosis; scleroderma; glomerular sclerosis; microvascular complications, for example, diabetic retinopathy; renal vascular hypertension; vasculopathy; neuropathy; complications resulting from diabetes, including nephropathy, vasculopathy, retinopathy and neuropathy, diseases of the coronary vessels, proteinuria, albumenuria, post-surgical hypertension, metabolic syndrome, obesity, restenosis following angioplasty, eye diseases and associated abnormalities including raised intra-ocular pressure, glaucoma, retinopathy, abnormal vascular growth and remodelling, angiogenesis-related disorders, such as neovascular age related macular degeneration; hyperaldosteronism, anxiety states, and cognitive disorders (Fisher N. D.; Hollenberg N. K. Expert Opin. Investig. Drugs. 2001, 10, 417-26).

Elevated levels of βamyloid, the product of the activity of the well-characterized aspartic protease β-secretase (BACE) activity on amyloid precursor protein, are widely believed to be responsible for the development and progression of amyloid plaques in the brains of Alzheimer's disease patients. The secreted aspartic proteases of Candida albicans are associated with its pathogenic virulence (Naglik, J. R.; Challacombe, S. J.; Hube, B. Microbiology and Molecular Biology Reviews 2003, 67, 400-428). The viruses HIV and HTLV depend on their respective aspartic proteases for viral maturation. Plasmodium falciparum uses plasmepsins I and II to degrade hemoglobin.

A pharmaceutical composition of the invention may, alternatively or in addition to a compound of Formula I, comprise a pharmaceutically acceptable salt of a compound of Formula I or a prodrug or pharmaceutically active metabolite of such a compound or salt and one or more pharmaceutically acceptable carriers therefor.

The compositions of the invention are aspartic protease inhibitors. Said compositions contain compounds having a mean inhibition constant (IC₅₀) against aspartic proteases of between about 5,000 nM to about 0.01 nM; preferably between about 50 nM to about 0.01 nM; and more preferably between about 5 nM to about 0.01 nM.

The compositions of the invention reduce blood pressure. Said compositions include compounds having an IC₅₀ for renin of between about 5,000 nM to about 0.01 nM; preferably between about 50 nM to about 0.01 nM; and more preferably between about 5 nM to about 0.01 nM.

The invention includes a therapeutic method for treating or ameliorating an aspartic protease mediated disorder in a subject in need thereof comprising administering to a subject in need thereof an effective amount of a compound of Formula I, or the enantiomers, diastereomers, or salts thereof or composition thereof.

Administration methods include administering an effective amount (i.e., a therapeutically effective amount) of a compound or composition of the invention at different times during the course of therapy or concurrently in a combination form. The methods of the invention include all known therapeutic treatment regimens.

“Prodrug” means a pharmaceutically acceptable form of an effective derivative of a compound (or a salt thereof) of the invention, wherein the prodrug may be: 1) a relatively active precursor which converts in vivo to a compound of the invention; 2) a relatively inactive precursor which converts in vivo to a compound of the invention; or 3) a relatively less active component of the compound that contributes to therapeutic activity after becoming available in vivo (i.e., as a metabolite). See “Design of Prodrugs”, ed. H. Bundgaard, Elsevier, 1985.

“Metabolite” means a pharmaceutically acceptable form of a metabolic derivative of a compound (or a salt thereof) of the invention, wherein the derivative is an active compound that contributes to therapeutic activity after becoming available in vivo.

“Effective amount” means that amount of active compound agent that elicits the desired biological response in a subject. Such response includes alleviation of the symptoms of the disease or disorder being treated. The effective amount of a compound of the invention in such a therapeutic method is from about 10 mg/kg/day to about 0.01 mg/kg/day, preferably from about 0.5 mg/kg/day to 5 mg/kg/day.

The invention includes the use of a compound of the invention for the preparation of a composition for treating or ameliorating an aspartic protease mediated chronic disorder or disease or infection in a subject in need thereof, wherein the composition comprises a mixture one or more compounds of the invention and an optional pharmaceutically acceptable carrier.

“Pharmaceutically acceptable carrier” means compounds and compositions that are of sufficient purity and quality for use in the formulation of a composition of the invention and that, when appropriately administered to an animal or human, do not produce an adverse reaction.

“Aspartic protease mediated disorder or disease” includes disorders or diseases associated with the elevated expression or overexpression of aspartic proteases and conditions that accompany such diseases.

An embodiment of the invention includes administering a renin inhibiting compound of Formula I or composition thereof in a combination therapy (U.S. Pat. No. 5,821,232, U.S. Pat. No. 6,716,875, U.S. Pat. No. 5,663,188, Fossa, A. A.; DePasquale, M. J.; Ringer, L. J.; Winslow, R. L. “Synergistic effect on reduction in blood pressure with coadministration of a renin inhibitor or an angiotensin-converting enzyme inhibitor with an angiotensin II receptor antagonist” Drug Development Research 1994, 33(4), 422-8) with one or more additional agents for the treatment of hypertension including α-blockers, β-blockers, calcium channel blockers, diuretics, natriuretics, saluretics, centrally acting antiphypertensives, angiotensin converting enzyme (ACE) inhibitors, dual ACE and neutral endopeptidase (NEP) inhibitors, angiotensin-receptor blockers (ARBs), aldosterone synthase inhibitor, aldosterone-receptor antagonists, or endothelin receptor antagonist.

α-Blockers include doxazosin, prazosin, tamsulosin, and terazosin.

β-Blockers for combination therapy are selected from atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, carvedilol, and their pharmaceutically acceptable salts.

Calcium channel blockers include dihydropyridines (DHPs) and non-DHPs. The preferred DHPs are selected from the group consisting of amlodipine, felodipine, ryosidine, isradipine, lacidipine, nicardipine, nifedipine, nigulpidine, niludipine, nimodiphine, nisoldipine, nitrendipine, and nivaldipine and their pharmaceutically acceptable salts. Non-DHPs are selected from flunarizine, prenylamine, diltiazem, fendiline, gallopamil, mibefradil, anipamil, tiapamil, and verampimil and their pharmaceutically acceptable salts.

A diuretic is, for example, a thiazide derivative selected from amiloride, chlorothiazide, hydrochlorothiazide, methylchlorothiazide, and chlorothalidon.

ACE inhibitors include alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril. Preferred ACE inhibitors are benazepril, enalpril, lisinopril, and ramipril.

Dual ACE/NEP inhibitors are, for example, omapatrilat, fasidotril, and fasidotrilat.

Preferred ARBs include candesartan, eprosartan, irbesartan, losartan, olmesartan, tasosartan, telmisartan, and valsartan.

Preferred aldosterone synthase inhibitors are anastrozole, fadrozole, and exemestane.

Preferred aldosterone-receptor antagonists are spironolactone and eplerenone.

A preferred endothelin antagonist is, for example, bosentan, enrasentan, atrasentan, darusentan, sitaxentan, and tezosentan and their pharmaceutically acceptable salts.

An embodiment of the invention includes administering an HIV protease inhibiting compound of Formula I or composition thereof in a combination therapy with one or more additional agents for the treatment of AIDS reverse transcriptase inhibitors, non-nucleoside reverse transcriptase inhibitors, other HIV protease inhibitors, HIV integrase inhibitors, entry inhibitors (including attachment, co-receptor and fusion inhibitors), antisense drugs, and immune stimulators.

Preferred reverse transcriptase inhibitors are zidovudine, didanosine, zalcitabine, stavudine, lamivudine, abacavir, tenofovir, and emtricitabine.

Preferred non-nucleoside reverse transcriptase inhibitors are nevirapine, delaviridine, and efavirenz.

Preferred HIV protease inhibitors are saquinavir, ritonavir, indinavir, nelfinavir, amprenavir, lopinavir, atazanavir, and fosamprenavir.

Preferred HIV integrase inhibitors are L-870,810 and S-1360.

Entry inhibitors include compounds that bind to the CD4 receptor, the CCR5 receptor or the CXCR4 receptor. Specific examples of entry inhibitors include enfuvirtide (a peptidomimetic of the HR2 domain in gp41) and sifurvitide.

A preferred attachment and fusion inhibitor is enfuvirtide.

An embodiment of the invention includes administering β-secretase inhibiting compound of Formula I or composition thereof in a combination therapy with one or more additional agents for the treatment of Alzheimer's disease including tacrine, donepezil, rivastigmine, galantamine, and memantine.

An embodiment of the invention includes administering a plasmepsin inhibiting compound of Formula I or composition thereof in a combination therapy with one or more additional agents for the treatment of malaria including artemisinin, chloroquine, halofantrine, hydroxychloroquine, mefloquine, primaquine, pyrimethamine, quinine, sulfadoxine

Combination therapy includes co-administration of the compound of the invention and said other agent, sequential administration of the compound and the other agent, administration of a composition containing the compound and the other agent, or simultaneous administration of separate compositions containing of the compound and the other agent.

The invention further includes the process for making the composition comprising mixing one or more of the present compounds and an optional pharmaceutically acceptable carrier; and includes those compositions resulting from such a process, which process includes conventional pharmaceutical techniques.

The compositions of the invention include ocular, oral, nasal, transdermal, topical with or without occlusion, intravenous (both bolus and infusion), and injection (intraperitoneally, subcutaneously, intramuscularly, intratumorally, or parenterally). The composition may be in a dosage unit such as a tablet, pill, capsule, powder, granule, liposome, ion exchange resin, sterile ocular solution, or ocular delivery device (such as a contact lens and the like facilitating immediate release, timed release, or sustained release), parenteral solution or suspension, metered aerosol or liquid spray, drop, ampoule, auto-injector device, or suppository; for administration ocularly, orally, intranasally, sublingually, parenterally, or rectally, or by inhalation or insufflation.

Compositions of the invention suitable for oral administration include solid forms such as pills, tablets, caplets, capsules (each including immediate release, timed release, and sustained release formulations), granules and powders; and, liquid forms such as solutions, syrups, elixirs, emulsions, and suspensions. Forms useful for ocular administration include sterile solutions or ocular delivery devices. Forms useful for parenteral administration include sterile solutions, emulsions, and suspensions.

The compositions of the invention may be administered in a form suitable for once-weekly or once-monthly administration. For example, an insoluble salt of the active compound may be adapted to provide a depot preparation for intramuscular injection (e.g., a decanoate salt) or to provide a solution for ophthalmic administration.

The dosage form containing the composition of the invention contains a therapeutically effective amount of the active ingredient necessary to provide a therapeutic effect. The composition may contain from about 5,000 mg to about 0.5 mg (preferably, from about 1,000 mg to about 0.5 mg) of a compound of the invention or salt form thereof and may be constituted into any form suitable for the selected mode of administration. The composition may be administered about 1 to about 5 times per day. Daily administration or post-periodic dosing may be employed.

For oral administration, the composition is preferably in the form of a tablet or capsule containing, e.g., 500 to 0.5 milligrams of the active compound. Dosages will vary depending on factors associated with the particular patient being treated (e.g., age, weight, diet, and time of administration), the severity of the condition being treated, the compound being employed, the mode of administration, and the strength of the preparation.

The oral composition is preferably formulated as a homogeneous composition, wherein the active ingredient is dispersed evenly throughout the mixture, which may be readily subdivided into dosage units containing equal amounts of a compound of the invention. Preferably, the compositions are prepared by mixing a compound of the invention (or pharmaceutically acceptable salt thereof) with one or more optionally present pharmaceutical carriers (such as a starch, sugar, diluent, granulating agent, lubricant, glidant, binding agent, and disintegrating agent), one or more optionally present inert pharmaceutical excipients (such as water, glycols, oils, alcohols, flavoring agents, preservatives, coloring agents, and syrup), one or more optionally present conventional tableting ingredients (such as corn starch, lactose, sucrose, sorbitol, talc, stearic acid, magnesium stearate, dicalcium phosphate, and any of a variety of gums), and an optional diluent (such as water).

Binder agents include starch, gelatin, natural sugars (e.g., glucose and beta-lactose), corn sweeteners and natural and synthetic gums (e.g., acacia and tragacanth). Disintegrating agents include starch, methyl cellulose, agar, and bentonite.

Tablets and capsules represent an advantageous oral dosage unit form. Tablets may be sugarcoated or film coated using standard techniques. Tablets may also be coated or otherwise compounded to provide a prolonged, control-release therapeutic effect. The dosage form may comprise an inner dosage and an outer dosage component, wherein the outer component is in the form of an envelope over the inner component. The two components may further be separated by a layer which resists disintegration in the stomach (such as an enteric layer) and permits the inner component to pass intact into the duodenum or a layer which delays or sustains release. A variety of enteric and non-enteric layer or coating materials (such as polymeric acids, shellacs, acetyl alcohol, and cellulose acetate or combinations thereof) may be used.

Compounds of the invention may also be administered via a slow release composition; wherein the composition includes a compound of the invention and a biodegradable slow release carrier (e.g., a polymeric carrier) or a pharmaceutically acceptable non-biodegradable slow release carrier (e.g., an ion exchange carrier).

Biodegradable and non-biodegradable slow release carriers are well known in the art. Biodegradable carriers are used to form particles or matrices which retain an active agent(s) and which slowly degrade/dissolve in a suitable environment (e.g., aqueous, acidic, basic and the like) to release the agent. Such particles degrade/dissolve in body fluids to release the active compound(s) therein. The particles are preferably nanoparticles (e.g., in the range of about 1 to 500 nm in diameter, preferably about 50-200 nm in diameter, and most preferably about 100 nm in diameter). In a process for preparing a slow release composition, a slow release carrier and a compound of the invention are first dissolved or dispersed in an organic solvent. The resulting mixture is added into an aqueous solution containing an optional surface-active agent(s) to produce an emulsion. The organic solvent is then evaporated from the emulsion to provide a colloidal suspension of particles containing the slow release carrier and the compound of the invention.

The compound of Formula I may be incorporated for administration orally or by injection in a liquid form such as aqueous solutions, suitably flavored syrups, aqueous or oil suspensions, flavored emulsions with edible oils such as cottonseed oil, sesame oil, coconut oil or peanut oil and the like, or in elixirs or similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone, and gelatin. The liquid forms in suitably flavored suspending or dispersing agents may also include synthetic and natural gums. For parenteral administration, sterile suspensions and solutions are desired. Isotonic preparations, which generally contain suitable preservatives, are employed when intravenous administration is desired.

The compounds may be administered parenterally via injection. A parenteral formulation may consist of the active ingredient dissolved in or mixed with an appropriate inert liquid carrier. Acceptable liquid carriers usually comprise aqueous solvents and other optional ingredients for aiding solubility or preservation. Such aqueous solvents include sterile water, Ringer's solution, or an isotonic aqueous saline solution. Other optional ingredients include vegetable oils (such as peanut oil, cottonseed oil, and sesame oil), and organic solvents (such as solketal, glycerol, and formyl). A sterile, non-volatile oil may be employed as a solvent or suspending agent. The parenteral formulation is prepared by dissolving or suspending the active ingredient in the liquid carrier whereby the final dosage unit contains from 0.005 to 10% by weight of the active ingredient. Other additives include preservatives, isotonizers, solubilizers, stabilizers, and pain-soothing agents. Injectable suspensions may also be prepared, in which case appropriate liquid carriers, suspending agents and the like may be employed.

Compounds of the invention may be administered intranasally using a suitable intranasal vehicle.

Compounds of the invention may also be administered topically using a suitable topical transdermal vehicle or a transdermal patch.

For ocular administration, the composition is preferably in the form of an ophthalmic composition. The ophthalmic compositions are preferably formulated as eye-drop formulations and filled in appropriate containers to facilitate administration to the eye, for example a dropper fitted with a suitable pipette. Preferably, the compositions are sterile and aqueous based, using purified water. In addition to the compound of the invention, an ophthalmic composition may contain one or more of: a) a surfactant such as a polyoxyethylene fatty acid ester; b) a thickening agents such as cellulose, cellulose derivatives, carboxyvinyl polymers, polyvinyl polymers, and polyvinylpyrrolidones, typically at a concentration n the range of about 0.05 to about 5.0% (wt/vol); c) (as an alternative to or in addition to storing the composition in a container containing nitrogen and optionally including a free oxygen absorber such as Fe), an anti-oxidant such as butylated hydroxyanisol, ascorbic acid, sodium thiosulfate, or butylated hydroxytoluene at a concentration of about 0.00005 to about 0.1% (wt/vol); d) ethanol at a concentration of about 0.01 to 0.5% (wt/vol); and e) other excipients such as an isotonic agent, buffer, preservative, and/or pH-controlling agent. The pH of the ophthalmic composition is desirably within the range of 4 to 8.

In the discussion below R, R¹, R², R³, X, Y, A, Q, E, and G are defined as described above for compounds of Formula I. In cases where the synthetic intermediates and final products of Formula I described below contain potentially reactive functional groups, for example amino, hydroxyl, thiol and carboxylic acid groups, that may interfere with the desired reaction, it may be advantageous to employ protected forms of the intermediate. Methods for the selection, introduction and subsequent removal of protecting groups are well known to those skilled in the art. (T. W. Greene and P. G. M. Wuts “Protective Groups in Organic Synthesis” John Wiley & Sons, Inc., New York 1999). In the discussion below all intermediates are assumed to be protected when necessary and protection/deprotection are generally not described.

In the first process of the invention, a compound of Formula I, in which a nitrogen atom that is part of A is attached to Q, is prepared by reaction of an amine of Formula II and an intermediate of Formula III:

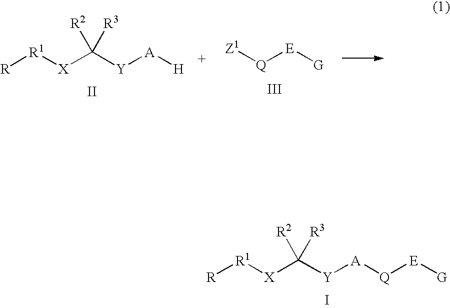

wherein Z¹ in III is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula II wherein H is attached to a nitrogen atom that is part of A are prepared from intermediates of Formula IV:

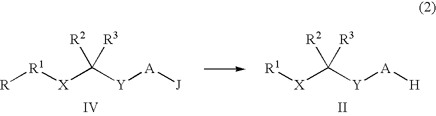

wherein J is an amine protecting group, including carbamate, amide, and sulfonamide protecting groups known in the art (T. W. Greene and P. G. M. Wuts “Protective Groups in Organic Synthesis” John Wiley & Sons, Inc., New York 1999).

Intermediates of Formula IV wherein R³=OH are prepared from ketone intermediates of formula V by addition of an organometallic reagent of formula VI, where M is for example Li, MgCl, MgBr, or MgI, to the carbonyl group of V:

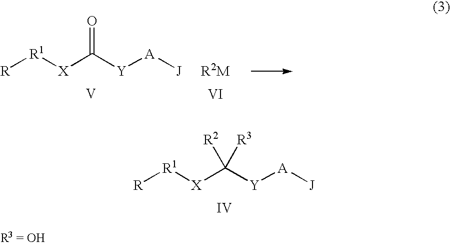

Intermediates of Formula IV wherein R³=H and R² is a group attached by an ether linkage are prepared from alcohol intermediates of formula VII by reaction with an alkylating agent under basic conditions or by reaction with an alcohol of formula R²OH under acidic conditions.

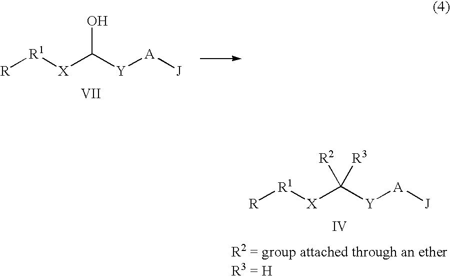

Alcohol intermediates of formula VII are prepared by reduction of ketone intermediates of formula V:

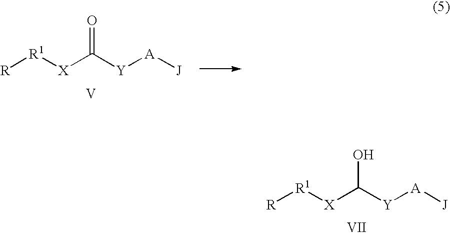

or by addition of an organometallic reagent of formula VIII, wherein M is, for example Li, MgCl, MgBr, or MgI, to an aldehyde of Formula IX:

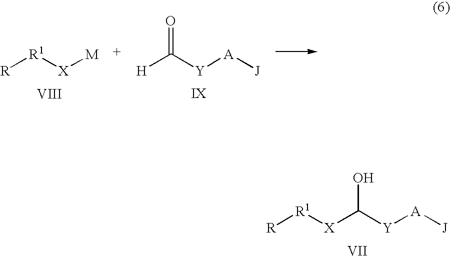

Ketone intermediates of formula V are prepared by the addition of an organometallic reagent of formula VIII, wherein M is Li, MgCl, MgBr, MgI, to a carboxylic acid derivative of formula X wherein Z² is an alkoxy, dialkylamino group, or an N-alkoxy-N-alkylamino group:

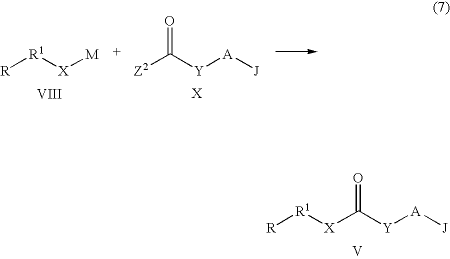

Intermediates of Formula V are also prepared from cuprate organometallic reagents of Formula XI wherein M is Li, MgCl, MgBr or MgI, and a carboxylic acid derivative of Formula X wherein Z² is an alkylthio, arylthio or heteroarylthio group:

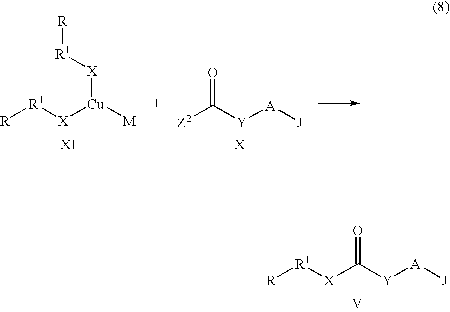

Intermediates of formula V are also prepared by oxidation of alcohol intermediates of formula VII:

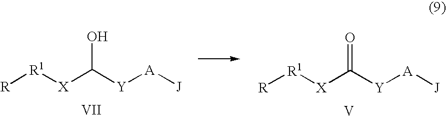

Intermediates of Formula IV, wherein R is an aryl or heteroaryl group, are also prepared by transition metal catalyzed cross coupling of organometallic intermediates of Formula XII, in which M is ZnCl, ZnBr, ZnI, B(OH)₂, pinocolatoboron, or Sn(n-Bu)₃, and intermediates of formula XIII, in which Z³ is a halide or trifluoromethanesulfonate:

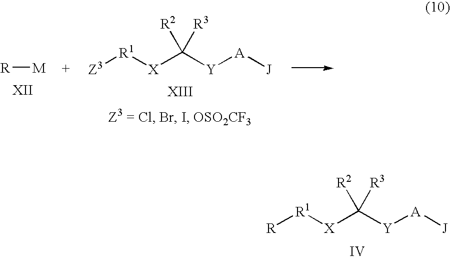

Intermediates of Formula IV, wherein the R is group attached to R¹ through an ether linkage, are also prepared by alkylation of intermediates of formula XIII, in which Z³ is a hydroxyl group with alkylating agents of formula XIV, wherein X is a halogen, alkanesulfonate, haloalkanesulfonate, or arenesulfonate leaving group:

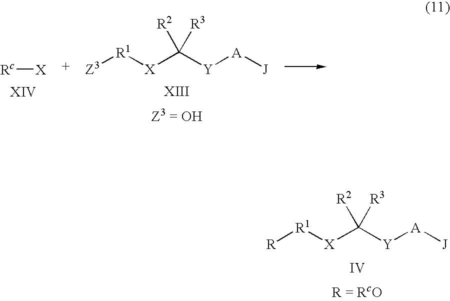

The intermediates of Formula XIII used in reaction schemes 10 and 11 are available by processes analogous to those described for IV (reaction schemes 3 and 4).

Intermediates of Formula IV wherein R² is attached to the molecule through a carbon atom and R³ is H are prepared from intermediates of Formula IV wherein R³ is OH in one step by deoxygenation, for example with Raney nickel, or in two steps by elimination of water followed by hydrogenation:

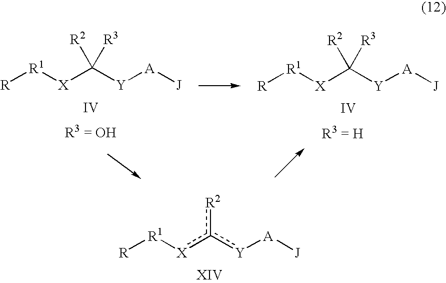

Intermediates of Formula III, wherein Q is Q1 attached to a carbon atom of E and Z, is alkanesulfonate, haloalkanesulfonate, carboxylate, arylsulfonate, or represents an active ester are prepared by activation of carboxylic acids of Formula XV:

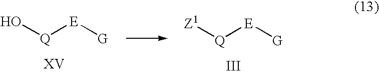

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula III are often prepared and used in situ without isolation.

Intermediates of Formula III, wherein Q is Q1 attached to a nitrogen atom that is part of E and Z¹ is halide, aryloxide, or an azole are prepared by reaction of amine intermediates of Formula XVI with phosgene, aryl chloroformates (e.g., p-nitrophenyl chloroformate or pentafluorophenyl chloroformate), or carbonyl diimidazole respectively:

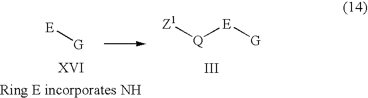

Intermediates of Formula III wherein Q is Q4, Q5, Q6, Q8, Q9 or Q10 attached to a nitrogen atom that is part of E are prepared by reaction of an amine intermediate of Formula XVI with an intermediate of Formula XVII wherein Z¹ is aryloxy, alkoxy, alkylthio, or arylthio:

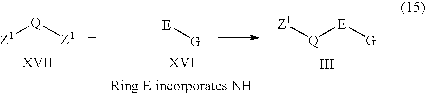

In the second process of the invention, a compound of Formula I, in which a nitrogen atom that is part of E is attached to Q, is prepared by reaction of an intermediate of Formula XVIII and an amine of Formula XVI:

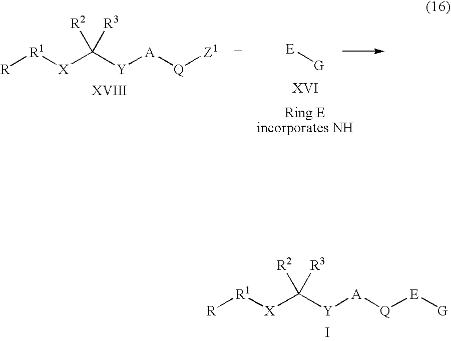

wherein Z¹ is as defined above.

Intermediates of Formula XVIII wherein Q is attached to a nitrogen atom of ring A and Q is Q1, Q4, Q5, Q6, Q8, Q9, or Q10 are prepared from amine intermediates of Formula II and intermediates of Formula XVII wherein Z¹ is halide, alkanesulfonate, haloalkanesulfonate, carboxylate, arylsulfonate, aryloxy, heteroaryloxy, azole, azolium salt, alkoxy, alkylthio, or arylthio:

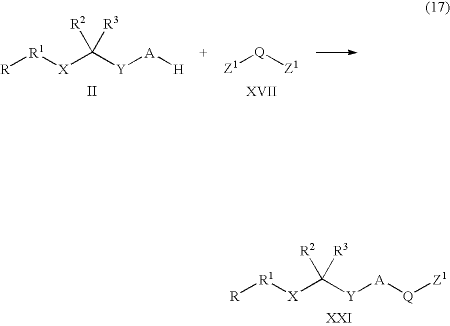

In the third process of the invention, a compound of Formula I in which R³ is hydroxy is prepared by addition of an organometallic species of Formula VI, wherein M¹ is for example Li, MgCl, MgBr, or MgI, to a ketone intermediate of Formula XIX:

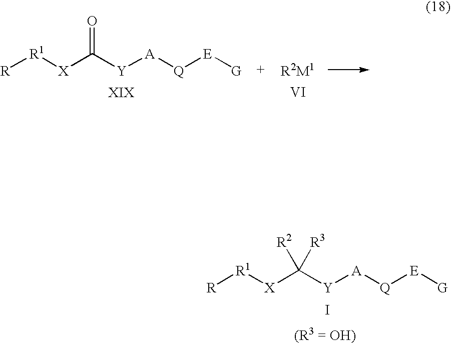

Ketone intermediates of Formula XIX are prepared by processes analogous to those shown for ketone intermediates of formula V in reaction schemes 7, 8, and 9.

In the fourth process of the invention, a compound of Formula I, in which R is an optionally substituted aromatic or heteroaromatic ring, is prepared by transition metal, especially palladium, catalyzed cross coupling of an organometallic species of Formula XX, wherein M² is for example B(OH)₂, B(OC(Me)₂C(Me₂)O), SnBu₃, or ZnBr, and an intermediate of Formula XXI wherein Z² is Cl, Br, I, or OSO₂CF₃:

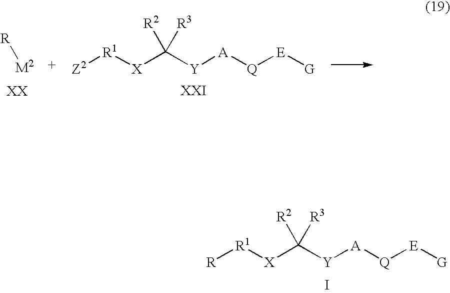

Intermediates of Formula XXI are prepared by processes analogous to those shown for compounds of Formula I in reaction schemes 1, 16, and 18.

In the fifth process of the invention, a compound of Formula I, in which R is an alkoxy, cycloalkoxy, cycloalkylalkoxy or arylalkoxy group, is prepared by reaction of an alkylating agent of Formula XXII, in which Z³ is chloride, bromide, iodide, methanesulfonate, arenesulfonate or trifluoromethanesulfonate and Rc is an alkyl, cycloalkyl, cycloalkylalkyl or arylalkyl, with a hydroxy compound of Formula XXIII:

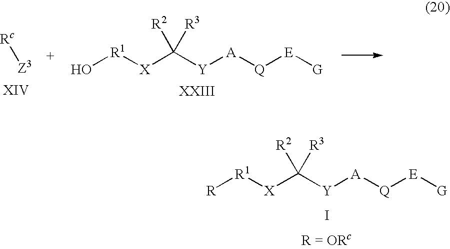

Intermediates of Formula XXIII are prepared by routes analogous to those shown for compounds of Formula I in reaction schemes 1 and 16.

In the sixth process of the invention, a compound of Formula I in which R² is attached through an ether linkage, R³ is H, A is an aromatic or heteroaromatic ring, and X and Y are single bonds is prepared from an alcohol of Formula XXIII and alcohol of Formula XXV in the presence of acid:

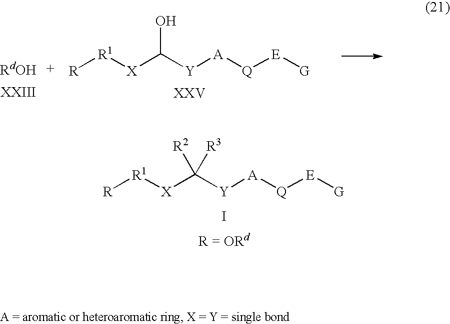

Alcohols of Formula XXV are prepared by reduction of ketones of XIX:

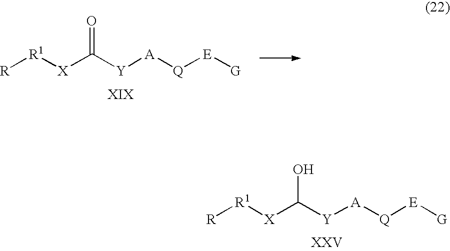

In the seventh process of the invention, a compound of Formula I in which G is an alkylamino group is prepared by reductive alkylation of a compound of Formula I in which G is amino with an aldehyde R^{a}CHO of Formula XXVI wherein R^{a} is alkyl with, for example, NaBH(OAc)₃ or NaBH₃CN:

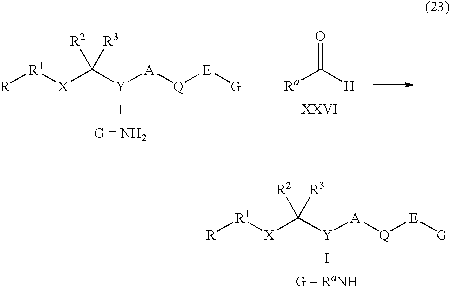

In the eighth process of the invention, a compound of Formula I wherein G is alkylamino is prepared from a compound of Formula I where G is NHMe by reductive alkylation with an aldehyde R^{a}CHO of Formula XXVI wherein R^{a} is alkyl with followed by N-demethylation with a nucleophilic species:

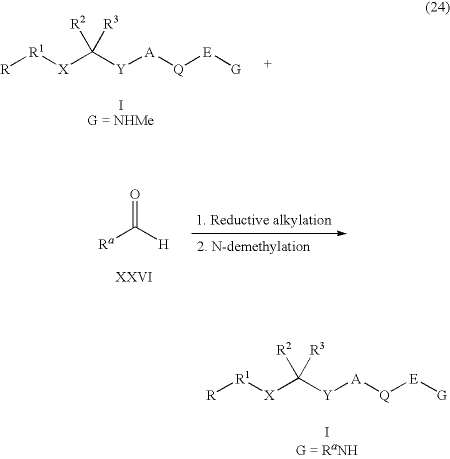

In the ninth process of the invention, a compound of Formula I in which R³=OH is treated with a nitrile XXVIII in which R^{a} is alkyl and a strong acid under the conditions of the Ritter reaction to afford a compound of Formula I in which R³=R^{a}CONH:

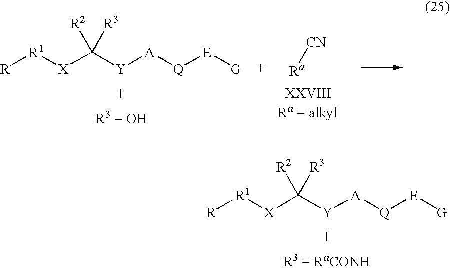

In the first process of the invention, a compound of Formula Ia, in which A¹ is a nitrogen atom is prepared by reaction of an amine of Formula IIa and an intermediate of Formula IIIa:

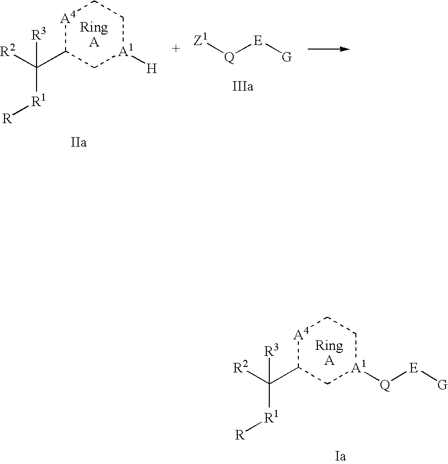

wherein Z¹ in III is a leaving group such as halide, alkanesulfonate, haloalkanesulfonate, carboxylate, arylsulfonate, aryloxy, heteroaryloxy, azole, azolium salt, alkoxy, alkylthio, or arylthio.

Intermediates of formula IIa in which A¹ is a nitrogen atom are prepared from intermediates of Formula IVa:

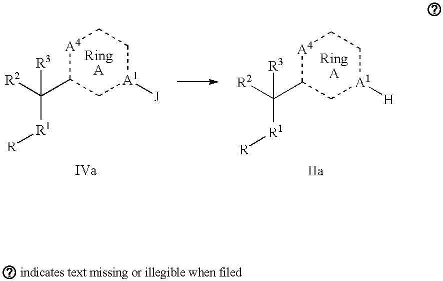

wherein J is an amine protecting group, including carbamate, amide and sulfonamide protecting groups known in the art (T. W. Greene and P. G. M. Wuts “Protective Groups in Organic Synthesis” John Wiley & Sons, Inc., New York 1999).

Intermediates of Formula IVa wherein R³=OH are prepared from ketone intermediates of formula Va by addition of an organometallic reagent of formula VIa, where M is for example Li, MgCl, MgBr, or MgI, to the carbonyl group of Va:

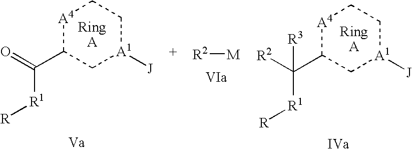

Intermediates of Formula IVa wherein R³=H and R² is a group attached by an ether linkage are prepared from alcohol intermediates of formula VIIa by reaction with an alkylating agent under basic conditions or by reaction with an alcohol under acidic conditions.

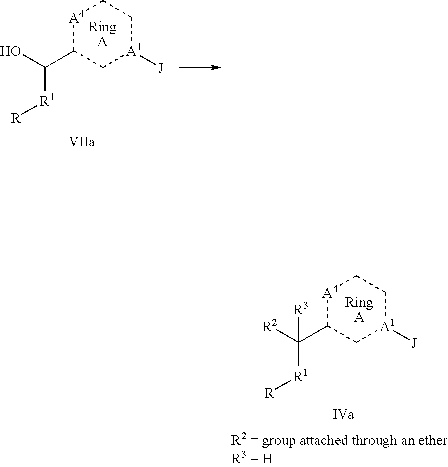

Alcohol intermediates of formula VIa are prepared by reduction of ketone intermediates of formula Va using reagents known in the art (Hanbook of Reagents for Organic Synthesis: Oxidizing and Reducing Reagents Ed. S. D. Burke and R. L. Danheiser, John Wiley & Sons, New York, 1999):

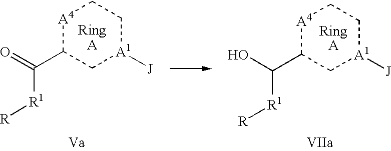

or by addition of an organometallic reagent of formula VIIIa, wherein M is, for example Li, MgCl, MgBr, or MgI, to an aldehyde of Formula IXa:

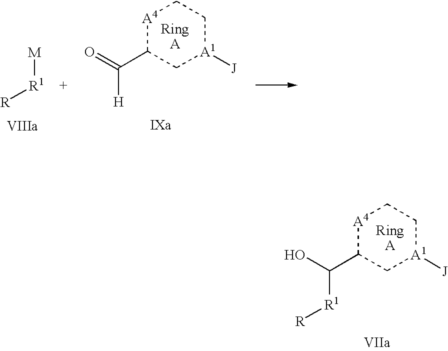

Ketone intermediates of formula Va are prepared by the addition of an organometallic reagent of formula VIIIa, wherein M is Li, MgCl, MgBr, MgI, to a carboxylic acid derivative of formula Xa wherein Z² is an alkoxy, dialkylamino group, or an N-alkoxy-N-alkylamino group:

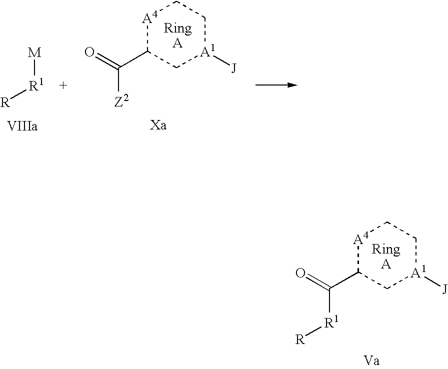

Intermediates of Formula Va are also prepared from cuprate organometallic reagents of Formula XIa wherein M is Li, MgCl, MgBr or MgI, and a carboxylic acid derivative of Formula Xa wherein Z² is an alkylthio, arylthio or heteroarylthio group:

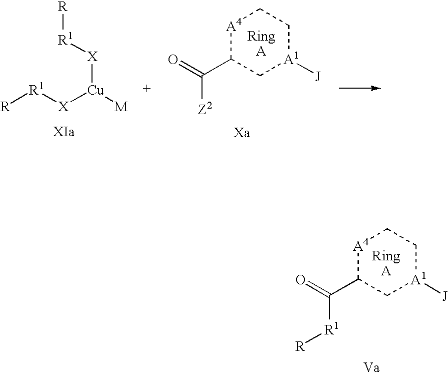

Intermediates of formula Va are also prepared by oxidation of alcohol intermediates of formula VIIa using reagents known in the art (Hanbook of Reagents for Organic Synthesis: Oxidizing and Reducing Reagents Ed. S. D. Burke and R. L. Danheiser, John Wiley & Sons, New York, 1999):

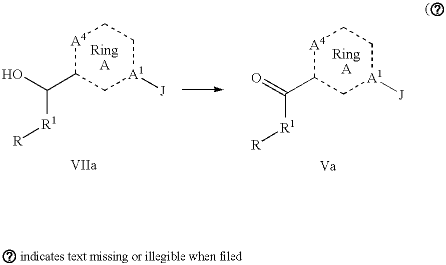

Intermediates of Formula IVa, wherein R is an aryl or heteroaryl group, are also prepared by transition metal catalyzed cross coupling of organometallic intermediates of Formula XIIa, in which M is ZnCl, ZnBr, ZnI, B(OH)₂, pinocolatoboron, or Sn(n-Bu)₃, and intermediates of formula XIIIa, in which Z³ is a halide or trifluoromethanesulfonate:

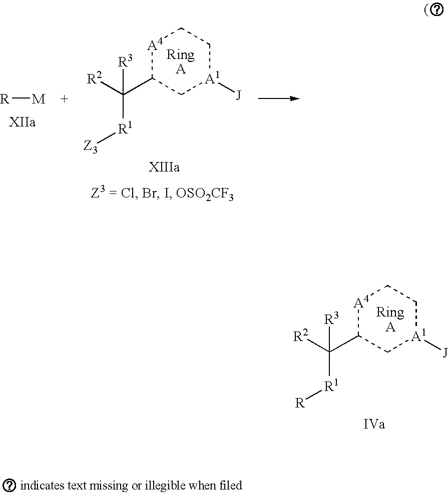

Intermediates of Formula IVa, wherein the R is group attached to R¹ through an ether linkage, are also prepared by alkylation of intermediates of formula XIIIa, in which Z³ is a hydroxyl group with alkylating agents of formula XIVa, wherein X is a halogen, alkanesulfonate, haloalkanesulfonate, or arenesulfonate leaving group:

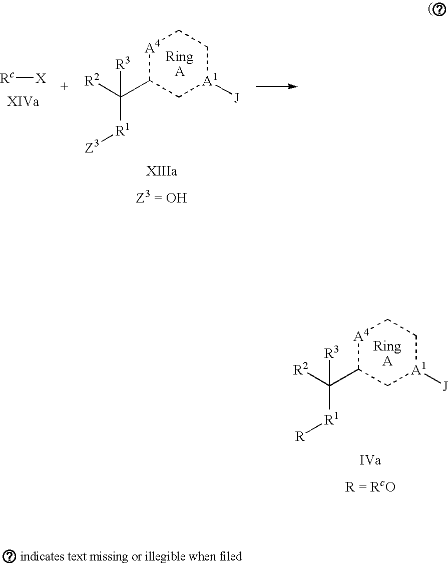

The intermediates of Formula XIIIa used in reaction schemes 10a and 11a are available by processes analogous to those described for IVa (reaction schemes 3a and 4a).

Intermediates of Formula IV wherein R² is attached to the molecule through a carbon atom and R³ is H are prepared from intermediates of Formula IV wherein R³ is OH in one step by deoxygenation, for example with Raney nickel, or in two steps by elimination of water followed by hydrogenation:

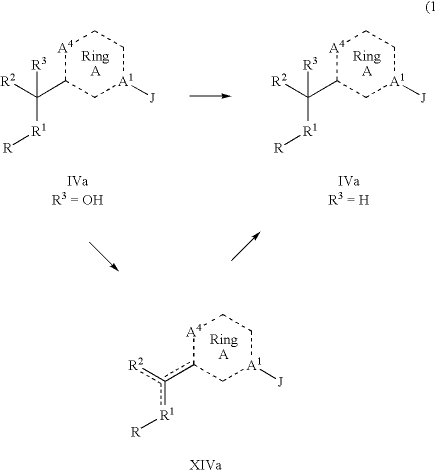

Intermediates of Formula IIIa, wherein Q is Q1 attached to a carbon atom of E and Z¹ is alkanesulfonate, haloalkanesulfonate, carboxylate, arylsulfonate, or represents an active ester are prepared by activation of carboxylic acids of Formula XVa:

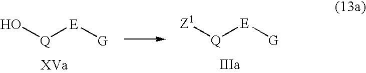

Reagents used to effect carboxylic activation are well known in the literature and include thionyl chloride and oxalyl chloride used to prepare acid chlorides, alkanesulfonyl chlorides used to prepare mixed anhydrides, alkyl chloroformates used to prepare mixed anhydrides, and carbodiimides used to prepare active esters. Intermediates of formula IIIa are often prepared and used in situ without isolation.

Intermediates of Formula IIIa, wherein Q is Q1 attached to a nitrogen atom that is part of E and Z¹ is halide, aryloxide, or an azole are prepared by reaction of amine intermediates of Formula XVI with phosgene, aryl chloroformates (e.g., p-nitrophenyl chloroformate or pentafluorophenyl chloroformate), or carbonyl diimidazole respectively:

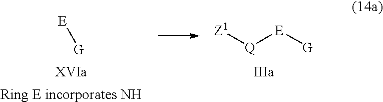

Intermediates of Formula IIIa wherein Q is Q4, Q5, Q6, Q8, Q9 or Q10 attached to a nitrogen atom that is part of E are prepared by reaction of an amine intermediate of Formula XVIa with an intermediate of Formula XVIa wherein Z¹ is aryloxy, alkoxy, alkylthio, or arylthio:

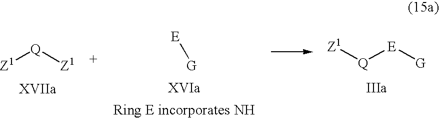

In the second process of the invention, a compound of Formula Ia, in which a nitrogen atom that is part of E is attached to Q, is prepared by reaction of an intermediate of Formula XVIIIa and an amine of Formula XVIa:

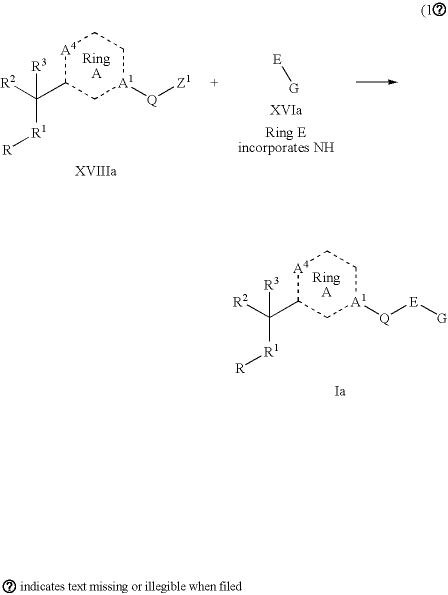

wherein Z¹ is as defined above.

Intermediates of Formula XVIIIa wherein Q is attached to a nitrogen atom of ring A and Q is Q1, Q4, Q5, Q6, Q8, Q9, or Q10 are prepared from amine intermediates of Formula IIa and intermediates of Formula XVIIa wherein Z¹ is halide, alkanesulfonate, haloalkanesulfonate, carboxylate, arylsulfonate, aryloxy, heteroaryloxy, azole, azolium salt, alkoxy, alkylthio, or arylthio:

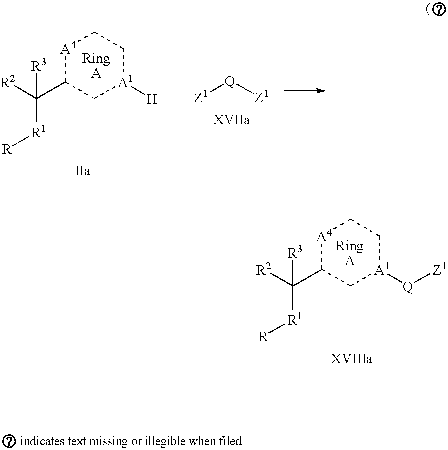

In the third process of the invention, a compound of Formula Ia in which R³ is hydroxy is prepared by addition of an organometallic species of Formula VIa, wherein M¹ is for example Li, MgCl, MgBr, or MgI, to a ketone intermediate of Formula XIX: a

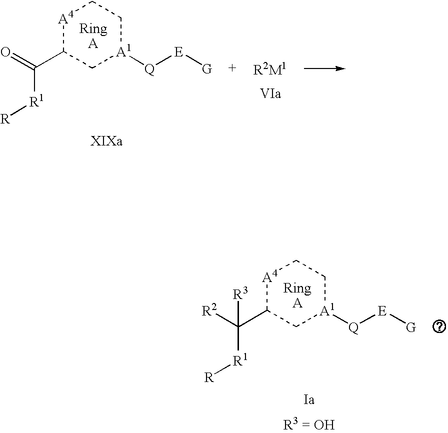

Ketone intermediates of Formula XIXa are prepared by processes analogous to those shown for ketone intermediates of formula Va in reaction schemes 7a, 8a, and 9a.

In the fourth process of the invention, a compound of Formula Ia, in which R is an optionally substituted aromatic or heteroaromatic ring, is prepared by transition metal, especially palladium, catalyzed cross coupling of an organometallic species of Formula XXa, wherein M² is for example B(OH)₂, B(OC(Me)₂C(Me₂)O), SnBu₃, or ZnBr, and an intermediate of Formula XXIa wherein Z² is Cl, Br, I, or OSO₂CF₃:

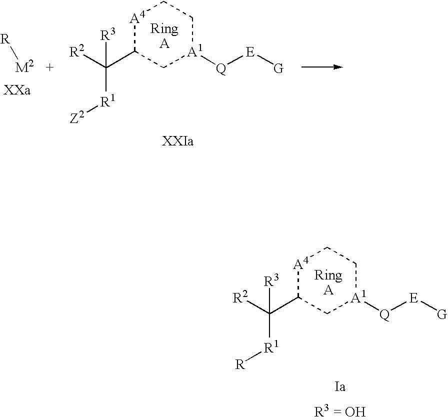

Intermediates of Formula XXIa are prepared by processes analogous to those shown for compounds of Formula I in reaction schemes 1a, 16a, and 18a.

In the fifth process of the invention, a compound of Formula Ia, in which R is an alkoxy, cycloalkoxy, cycloalkylalkoxy or arylalkoxy group, is prepared by reaction of an alkylating agent of Formula XIVa, in which Z³ is chloride, bromide, iodide, methanesulfonate, arenesulfonate or trifluoromethanesulfonate and Rc is an alkyl, cycloalkyl, cycloalkylalkyl or arylalkyl group, with a hydroxy compound of Formula XXIa:

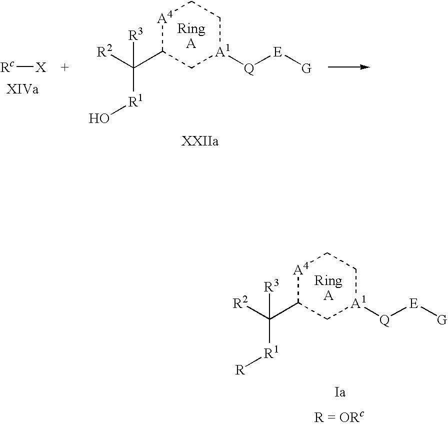

Intermediates of Formula XXIIa are prepared by routes analogous to those shown for compounds of Formula Ia in reaction schemes 1a and 16a.

In the sixth process of the invention, a compound of Formula Ia in which R² is attached through an ether linkage, R³ is H and Ring A is benzene ring, is prepared from an alcohol of Formula XXIIIa and alcohol of Formula XXIVa in the presence of acid:

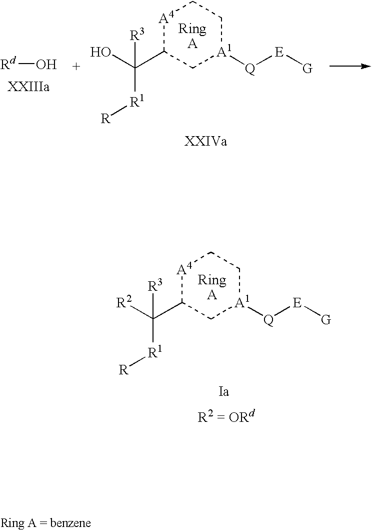

Alcohols of Formula XXIVa wherein R³ is hydrogen are prepared by reduction of ketones of XIXa. Alcohols of Formula XXIVa wherein R³ is an alkyl group are prepared by addition of an organometallic reagent R³M, wherein M=Li, MgCl, MgBr or MgI to ketones of XIXa:

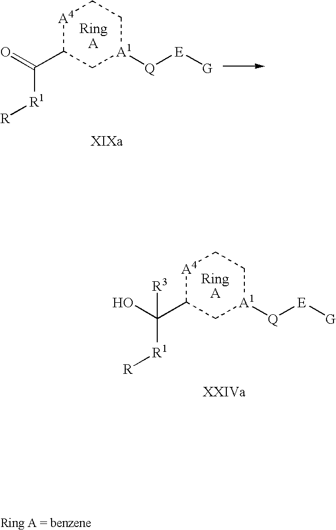

In the seventh process of the invention, a compound of Formula Ia in which G is an alkylamino or alkylaminoalkyl group is prepared by reductive alkylation of a compound of Formula Ia in which G is amino with an aldehyde R^{a}CHO of Formula XXVa wherein R^{a} is alkyl using, for example, NaBH(OAc)₃ or NaBH₃CN as reducing agent:

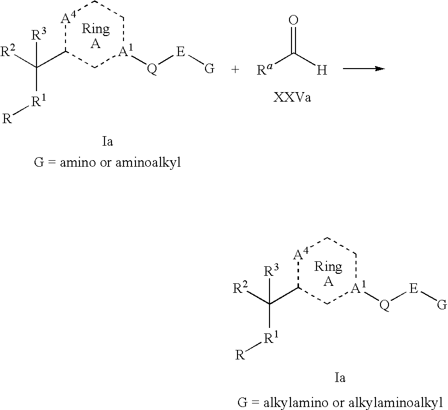

In the eighth process of the invention, a compound of Formula Ia wherein G is alkylamino is prepared from a compound of Formula Ia where G is methylamino by reductive alkylation with an aldehyde of formula XXVa wherein R^{a} is alkyl followed by N-demethylation with a nucleophilic species:

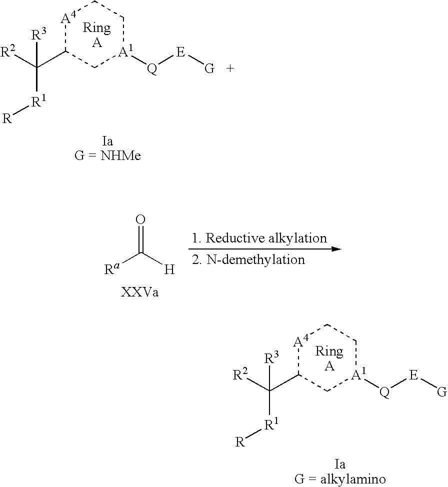

In the ninth process of the invention, a compound of Formula Ia in which R³=OH is treated with a nitrile XXVIa in which R^{a} is alkyl and a strong acid under the conditions of the Ritter reaction to afford a compound of Formula Ia in which R³=R^{a}CONH:

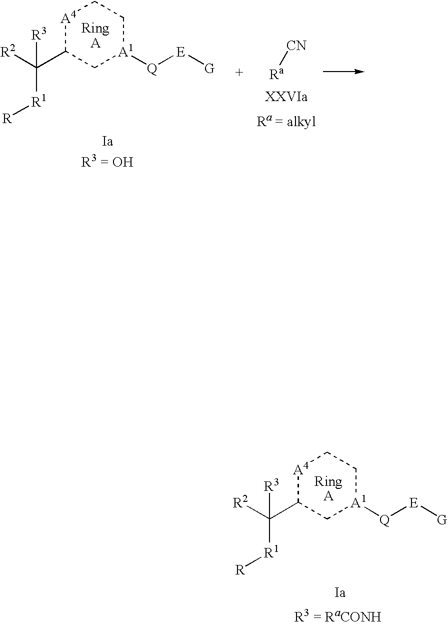

The invention is further defined by reference to the examples, which are intended to be illustrative and not limiting.

Representative compounds of the invention can be synthesized in accordance with the general synthetic schemes described above and are illustrated in the examples that follow. The methods for preparing the various starting materials used in the schemes and examples are well within the knowledge of persons skilled in the art.

The following abbreviations have the indicated meanings:

| Abbreviation | Meaning |
|---|---|
| aq | aqueous |
| Boc | tert-butoxy carbonyl or t-butoxy carbonyl |
| (Boc)₂O | di-tert-butyl dicarbonate |
| brine | saturated aqueous NaCl |
| CH₂Cl₂ | methylene chloride |
| CH₃CN or MeCN | acetonitrile |
| Cpd | compound |
| d | day |
| DBU | 1,8-diazabicyclo[5.4.0]undec-7-ene |
| DIEA | N,N-diisopropylethylamine |
| DMAP | 4-(dimethylamino)pyridine |
| DMF | N,N-dimethylformamide |
| DMPU | 1,3-dimethyl-3,4,5,6-tetrahydro-2(1H)-pyrimidinone |
| EDC•HCl | 1-[3-(dimethylamino)propyl]-3-ethylcarbodiimide |
| | hydrochloride |
| equiv | equivalents |
| Et | ethyl |
| Et₂O | ethyl ether |
| EtOAc | ethyl acetate |
| Fmoc | 1-[[(9H-fluoren-9-ylmethoxy)carbonyl]oxy]- |
| Fmoc-OSu | 1-[[(9H-fluoren-9-ylmethoxy)carbonyl]oxy]-2,5- |
| | pyrrolidinedione |
| h, hr | hour |
| HOBt | 1-hydroxybenzotriazole |
| HATU | 2-(7-Aza-1H-benzotriazole-1-yl)-1,1,3,3- |
| | tetramethyluronium hexafluorophosphate |
| HBTU | 2-(1H-Benzotriazole-1-yl)-1,1,3,3-tetramethyluronium |
| | hexafluorophosphate |
| KHMDS | potassium hexamethyldisilazane |
| LAH or LiAlH₄ | lithium aluminum hydride |
| LC-MS | liquid chromatography-mass spectroscopy |
| LHMDS | lithium hexamethyldisilazane |
| Me | methyl |
| MeCN | aceronitrile |
| MeOH | methanol |
| MsCl | methanesulfonyl chloride |
| min | minute |
| MS | mass spectrum |
| NaH | sodium hydride |
| NaHCO₃ | sodium bicarbonate |
| NaN₃ | sodium azide |
| NaOH | sodium hydroxide |
| Na₂SO₄ | sodium sulfate |
| NMP | N-methylpyrrolidinone |
| Pd₂(dba)₃ | tris(dibenzylideneacetone)dipalladium(0) |
| Ph | phenyl |
| rt | room temperature |
| satd | saturated |
| SOCl₂ | thionyl chloride |
| TBAF | tetrabutylammonium fluoride |
| TEA | triethylamine or Et₃N |
| TEAF | tetraethylammonium fluoride |
| TEMPO | 2,2,6,6-tetramethyl-1-piperidinyloxy, free radical |
| Teoc | 1-[2-(trimethylsilyl)ethoxycarbonyloxy]- |
| Teoc-OSu | 1-[2-(trimethylsilyl)ethoxycarbonyloxy]pyrrolidin- |
| | 2,5-dione |
| TFA | trifluoroacetic acid |
| THF | tetrahydrofuran |
| TMSCl | chlorotrimethylsilane or trimethylsilyl chloride |
| t_{R} | retention time |

### LC-MS Methods

Method 1 [LC-MS (3 min)]

Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 2.0 | 10 | 90 |
| 2.4 | 10 | 90 |
| 2.5 | 90 | 10 |
| 3.0 | 90 | 10 |

Method 2 [LC-MS (16 min)]

Column: Chromolith SpeedRod, RP-18e, 50×4.6 mm; Mobil phase: A: 0.01% TFA/water, B: 0.01% TFA/CH₃CN; Flow rate: 1 mL/min; Gradient:

| Time (min) | A % | B % |
|---|---|---|
| 0.0 | 90 | 10 |
| 14.0 | 10 | 90 |
| 15.0 | 10 | 90 |
| 15.1 | 90 | 10 |
| 16.0 | 90 | 10 |

Method 3 [Instrument 1]

Analytical LC-MS was conducted on an Agilent 1100 Series LC/MSD SL or VL using electrospray positive [ES+ve to give MH⁺] equipped with a Sunfire C₁₈ 5.0 μm column (3.050 mm×50 3.0 mm, i.d.), eluting with 0.05% TFA in water (solvent A) and 0.05% TFA in acetonitrile (solvent B), using the following elution gradient 10%-99% (solvent B) over 3.0 min and holding at 99% for 1.0 min at a flow rate of 1.0 ml/min.

Method 4 [Instrument 2]

Analytical LC-MS was conducted on an PE Sciex API 150 single quadrupole mass spectrometer using electrospray positive [ES+ve to give MH+] equipped with a Aquasil CIS 5 μm column (1 mm×40 mm), eluting with 0.02% TFA in water (solvent A) and 0.018% TFA in acetonitrile (solvent B), using the following elution gradient 4.5%-90% (solvent B) over 3.2 min and holding at 90% for 0.4 min at a flow rate of 0.3 ml/min.

Method 5

Analytical LC-MS was conducted on an Agilent 1200 Series LC/MSD VL using electrospray positive [ES+ve to give MH⁺] equipped with a YMC C₁₈ 5.0 μm column (2.0 mm×50, 2.0 mm, i.d.), eluting with 0.0375% TFA in water (solvent A) and 0.01875% TFA in acetonitrile (solvent B), using the following elution gradient 10%-80% (solvent B) over 2.0 min and holding at 80% for 0.5 min at a flow rate of 1.0 ml/min.

### Chiral HPLC Method

Column: Chiralpak AD-H, 0.46 cm×25 cm

### Solvent A: 0.025% Diethylamine in Hexane

### Solvent B: Isopropanol

Flow rate: 1 mL/min.
40 min. run

### Gradient:

| Time (min) | A(%) | B(%) |
|---|---|---|
| 0 | 95 | 5 |
| 40 | 90 | 10 |

The following procedures describe preparation of intermediates used in the synthesis of compounds of Formula I

### Preparation 1

### Weinreb Amide

### (R)-tert-butyl 3-(N-methoxy-N-methylcarbamoyl)piperidine-1-carboxylate

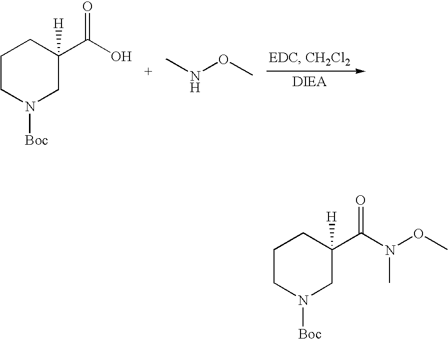

(R)-1-(tert-butoxycarbonyl)piperidine-3-carboxylic acid (25 g, 0.11 mol, 1.0 equiv), N,O-dimethylhydroxylamine hydrochloride, (10.5 g, 0.14 mol, 1.25 equiv), EDC.HCl (26.3 g, 0.14 mol, 1.25 equiv) and DIEA (48 mL, 0.28 mol, 2.5 equiv) were dissolved in CH₂Cl₂ (400 mL) and stirred overnight at rt. The reaction mixture was diluted with EtOAc, washed with 5% aq HCl (2×150 mL), satd aq NaHCO₃ (150 mL), brine (100 mL), and dried over Na₂SO₄. Concentration afforded (R)-tert-butyl 3-(N-methoxy-N-methylcarbamoyl)-piperidine-1-carboxylate (24.42 g, 82%) as a clear oil.

### Preparation 2

### Halodiphenyl Ethers from Halophenols and Benzeneboronic Acids

### 1-(3-Fluorophenoxy)-2-bromobenzene

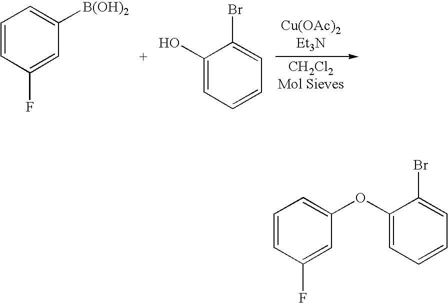

To a stirred solution of 3-fluorophenylboronic acid (2.10 g, 15 mmol), 2-bromophenol (1.77 g, 10 mmol) and Cu(OAc)₂ (0.93 g, 5 mmol) in anhydrous CH₂Cl₂ (25 mL) was added activated 4 Å molecular sieves (˜0.1 g), followed by anhydrous Et₃N (3.5 mL, 25 mmol). The resulting dark green solution was stirred at rt for 48 h. The mixture was evaporated under reduced pressure and the residue was washed several times with Et₂O (˜150 mL). The Et₂O solution was washed with satd aq NH₄Cl, and 1 N aq HCl. The organic layer was evaporated and the crude product was purified by flash column chromatography to give 1-(3-fluorophenoxy)-2-bromobenzene (1.28 g, 48%) as clear oil.

The following halodiphenyl ethers were prepared following the procedure described above.

| Halodiphenyl ether | Phenol | Boronic Acid |
|---|---|---|
| 1-(2-ethylphenoxy)-2- | 2-bromophenol | 2-ethylphenylboronic acid |
| bromobenzene | | |
| 1-(4-fluorophenoxy)-2- | 2-bromophenol | 4-fluorophenylboronic acid |
| bromobenzene | | |
| 1-(2-bromophenoxy)-3- | 2-bromophenol | 3-methylphenylboronic acid |
| methylbenzene | | |
| 2-(o-tolyloxy)-1-bromo-3- | 2-bromo-6-methylphenol | 2-methylphenylboronic acid |
| methylbenzene | | |
| 2-(o-tolyloxy)-1-bromo-3,5- | 2-bromo-4,6-difluorophenol | 2-methylphenylboronic acid |
| difluorobenzene | | |
| 1-(4-fluoro-2-methylphenoxy)- | 2-bromophenol | 4-fluoro-2- |
| 2-bromobenzene | | methylphenylboronic acid |
| 1-(5-fluoro-2- | 2-bromophenol | 5-fluoro-2- |
| methylphenoxy)-2- | | methylphenylboronic acid |
| bromobenzene | | |
| 1-chloro-3-fluoro-2- | 2-chloro-6-fluorophenol | phenylboronic acid |
| phenoxybenzene | | |
| 2-(p-tolyloxy)-1-chloro-3- | 2-chloro-6-fluorophenol | 4-methylphenylboronic acid |
| fluorobenzene | | |
| 2-bromo-4-fluoro-1- | 2-bromo-4-fluorophenol | phenylboronic acid |
| phenoxybenzene | | |
| 1-bromo-3-fluoro-2-(o- | 2-bromo-6-fluorophenol | 2-methylphenylboronic acid |
| tolyloxy)benzene | | |
| 2-bromo-4-fluoro-1-(4- | 2-bromo-4-fluorophenol | 4-fluorophenylboronic acid |
| fluorophenoxy)benzene | | |
| 1-bromo-3-chloro-2-[(3- | 2-bromo-6-chlorophenol | 3-ethylphenylboronic acid |
| ethylphenyl)oxy]benzene | | |
| 1-bromo-3-chloro-2-[(2- | 2-bromo-6-chlorophenol | 2-methylphenylboronic acid |
| methylphenyl)oxy]benzene | | |
| 1-bromo-3-chloro-2-[(2- | 2-bromo-6-chlorophenol | 2-ethylphenylboronic acid |
| ethylphenyl)oxy]benzene | | |

### Preparation 3

### Halodiphenyl Ethers from Phenoxyanilines

### 1-(O-tolyloxy)-2-iodobenzene

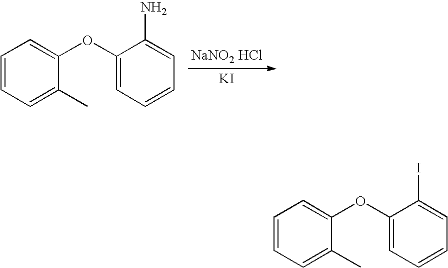

To a solution of 2-(o-tolyloxy)aniline (40 g, 0.2 mol) in 1N aq HCl (400 mL, 0.4 mol, 2 equiv) cooled to 0° C. was added dropwise a solution of NaNO₂ (18 g, 0.26 mol, 1.3 equiv) in water (520 ml). The mixture was stirred for 1 h at 0° C. and a solution of KI (83 g, 0.5 mol, 2.5 equiv) in water (500 mL) was added dropwise with vigorous stirring. After 0.5 h the mixture was warmed to 90-100° C. for 1 h, cooled to rt and washed with satd NaHSO₃ until the aqueous layer become clear. The mixture was extracted with EtOAc (3×200 mL) and the combined organic layers were washed with aq Na₂S₂O₄ and dried over Na₂SO₄. After evaporation of the solvent, the solution was passed through a short silica gel column to afford 1-(o-tolyloxy)-2-iodobenzene (40.0 g, 65%).

### Preparation 4

### Halodiphenyl Ethers from Phenols and Fluoronitrobenzenes

### 1-(2-Iodophenoxy)-2-chlorobenzene

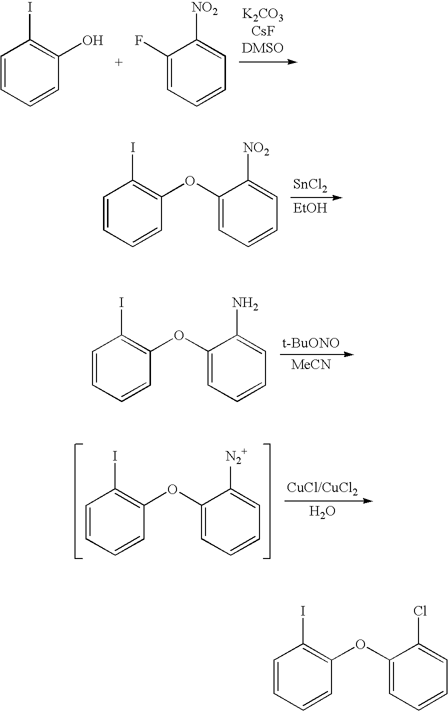

### Step 1. 1-(2-Iodophenoxy)-2-nitrobenzene

To a solution of 2-iodophenol (11.82 g, 52.7 mmol) and 1-fluoro-2-nitrobenzene (5.0 g, 35.1 mmol) in DMSO (50 mL was added K₂CO₃ (14.5 g, 105.3 mmol), followed by CsF (8.0 g, 52.7 mmol). The resulting suspension was stirred at 50° C. until no starting material remained (˜5 h), cooled to rt and partitioned between water (50 mL) and CH₂Cl₂ (50 mL). The water layer was separated and extracted with CH₂Cl₂ (2×10 mL). The combined organic layers were washed with 1 aq N NaOH (10 mL) and brine, and dried over Na₂SO₄. Solvent was removed under vacuum to give 1-(2-iodophenoxy)-2-nitrobenzene (11.2 g, 93%) as an oil, which was used for next step without purification.

### Step 2. 2-(2-Iodophenoxy)benzenamine

A solution of 1-(2-iodophenoxy)-2-nitrobenzene (9.60 g, 28.1 mmol) and SnCl.2H₂O (13.0 g, 56.0 mmol) in ethanol (25 mL) and water (5 mL) was refluxed until no starting material remained (˜1 h). The ethanol was removed in vacuo and the aq layer was basified to pH>10 and extracted with CH₂Cl₂ (4×10 mL). The combined organic layers were dried over Na₂SO₄, and the solvent was removed to give a crude 2-(2-Iodophenoxy)benzenamine (8.57 g, 98%), which was used for the next step without purification.

### Step 3. 1-(2-Iodophenoxy)-2-chlorobenzene

A solution of crude 2-(2-iodophenoxy)benzenamine (8.57 g, 27.6 mmol) in MeCN (60 mL) was cooled to 0° C. and treated with HBF₄ (54 wt % in Et₂O, 4.93 mL, 35.9 mmol). The reaction mixture was stirred at 0° C. for 5 min and of t-BuONO (4.10 g, 35.9 mmol) was added dropwise. The resulting mixture was stirred at 0° C. for 10 min, cooled to −20° C., and added to a solution of CuCl (41 g, 414.1 mmol) and CuCl₂ (70 g, 414.1 mmol) in water (500 mL) at 0° C. The mixture was stirred vigorously at 25° C. for 2 h, and partitioned between EtOAc and water. The water layer was extracted with EtOAc (3×10 mL) and the combined organic layers were washed with brine, dried over Na₂SO₄ and concentrated under vacuum. Flash column chromatography gave 1-(2-iodophenoxy)-2-chlorobenzene (5.35 g, 58%).

The following halodiphenyl ethers were prepared following the procedures described above using the starting materials and reagents indicated:

| | | Halide |
|---|---|---|
| Halopdiphenyl ether | Phenol in Step 1 | in Step 3 |
| 1-(2-iodophenoxy)-2- | 2-(trifluoromethyl)phenol | KI |
| (trifluoromethyl)benzene | | |
| 1-(2-iodophenoxy)-2- | 2-fluorophenol | KI |
| fluorobenzene | | |
| 2-[(2-bromophenyl)oxy]-1,3- | 2,6-dimethylphenol | CuBr/CuBr₂ |
| dimethylbenzene | | |
| 2-[(2-bromophenyl)oxy]-1- | 2-chloro-6-methylphenol | CuBr/CuBr₂ |
| chloro-3-methylbenzene | | |

### Preparation 5

### Piperidines from Weinreb Amides and Metallated Diphenyl Ether

### (S)-5-methoxy-1-(2-phenoxyphenyl)-1-((R)-piperidin-3-yl)pentan-1-ol

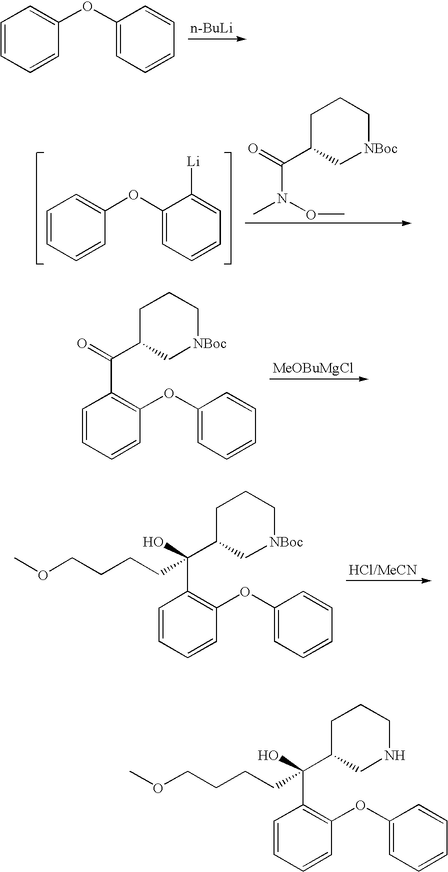

### Step 1. 2-(Phenoxy)phenyllithium

To a solution of diphenyl ether (8.60 g, 50.0 mmol) in Et₂O (75 mL) was added n-BuLi (1.6 M in hexane, 32.8 mL, 52.5 mmol). The mixture was refluxed for 48 h, and the resulting solution of 2-(phenoxy)phenyllithium was used in the next step without any further analysis.

### Step 2. (3R)-1-tert-butoxycarbonyl)-3-(2-phenoxybenzoyl)piperidine

To a solution of (R)-tert-butyl 3-(N-methoxy-N-methylcarbamoyl)piperidine-1-carboxylate (4.40 g, 16.2 mmol) in anhydrous THF (18 mL) at −10° C., was added dropwise the solution of 2-phenoxyphenyllithium prepared in Step 1 (80 mL, 32 mmol). The mixture was then warmed to rt, and stirred until no starting material remained (˜30 min). The reaction was quenched with 1 N HCl (˜30 mL) and extracted with Et₂O (4×10 mL). The combined organic layers were washed with satd aq NaHCO₃ and brine, and dried over Na₂SO₄. The solvent was removed to give (3R)-1-(tert-butoxycarbonyl)-3-(2-phenoxybenzoyl)piperidine (7.44 g, quantitative).

### Step 3. (R)-tert-Butyl 3-((S)-1-hydroxy-5-methoxy-1-(2-phenoxyphenyl)pentyl)piperidine-1-carboxylate

To a solution of (3R)-1-tert-butoxycarbonyl)-3-(2-phenoxybenzoyl)piperidine (6.17 g, 16.2 mmol) in THF (30 mL) at −10° C. was added dropwise 2.54 M 4-methoxybutylmagnesium chloride in THF (15 mL, 38 mmol). The resulting solution was warmed to rt slowly, and stirred over night. The reaction was quenched with satd NH₄Cl (10 mL) and extracted with Et₂O (4×10 mL). The combined organic layers were washed with water and brine. The solvent was removed and the residue was purified by flash chromatography to give (R)-tert-butyl 3-((S)-1-hydroxy-5-methoxy-1-(2-phenoxyphenyl)pentyl)piperidine-1-carboxylate (1.97 g, 26% from (R)-tert-butyl 3-(N-methoxy-N-methylcarbamoyl)piperidine-1-carboxylate).

### Step 4. (S)-5-Methoxy-1-(2-phenoxyphenyl)-1-((R)-piperidin-3-yl)pentan-1-ol

To a solution of (R)-tert-butyl 3-(S)-1-hydroxy-5-methoxy-1-(2-phenoxyphenyl)pentyl)piperidine-1-carboxylate (1.97 g, 4.19 mmol) in MeCN (100 mL) was added 2 N aq HCl (100 mL) slowly at rt. The resulting solution was stirred at rt until no starting material remained (˜16 h), basified to pH=10 with 10 N aq NaOH, and evaporated under reduced pressure to remove MeCN. The aq layer was extracted with CH₂Cl₂ (4×10 mL). The combined organic layers were washed with brine and dried over Na₂SO₄. The solvent was removed in vacuo to afford (S)-5-methoxy-1-(2-phenoxyphenyl)-1-((R)-piperidin-3-yl)pentan-1-ol (1.56 g, quantitative) as a free amine.

The following piperidines were prepared following procedures analogous to those described above:

(S)-1-(2-fluoro-5-(4-fluorophenoxy)phenyl)-5-methoxy-1-((R)-piperidin-3-yl)pentan-1-ol using 4,4′-difluorodiphenyl ether in Step 1.

### Preparation 6

### Piperidines from Weinreb Amides and 2-Bromophenols

### (S)-5-methoxy-1-(2-(2,2-(dimethyl)propoxy)phenyl)-1-((R)-piperidin-3-yl)pentan-1-ol hydrochloride

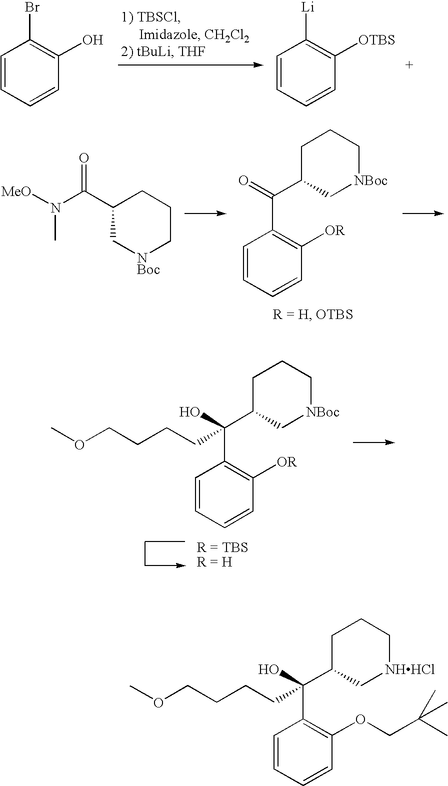

### Step 1. Bromo-2-[(tert-butyl)dimethylsiloxy]benzene

A solution of 2-bromophenol (5 mL, 47 mmol), imidazole (8 g, 118 mmol) and tert-butyldimethylsilyl chloride (8.6 g, 57 mmol) in DMF (50 mL) was stirred at rt overnight. The reaction was treated with water (150 mL) and extracted with Et₂O (4×25 mL). The organic phase was washed with 50% aq lithium chloride solution twice, dried over MgSO₄ and filtered. The solvent was evaporated and the crude product was purified by filtration through silica gel, washing with 1:1 EtOAc/hexanes to afford bromo-2-[(tert-butyl)dimethylsiloxy]benzene (13.4 g, 99%).

### Step 2. 2-(S)-1-hydroxy-5-methoxy-1-((R)—N-Boc-piperidin-3-yl)pentyl)[tert-butyldimethylsiloxy]benzene

A solution of bromo-2-[(tert-butyl)dimethylsiloxy]benzene (2.1 g, 7.4 mmol) in Et₂O (35 mL) was cooled to −78° C. and treated with 1.7 M tert-butyllithium in hexanes (8.6 mL, 15 mmol). The reaction was stirred for 30 min and a solution of (R)-tert-butyl 3-(N-methoxy-N-methylcarbamoyl)piperidine-1-carboxylate (1.0 g, 3.7 mmol) in Et₂O was added slowly. The reaction was allowed to stir and warm to rt over a two-hour period. Saturated aq ammonium chloride was added to quench the reaction. The aq phase was extracted with Et₂O three times. The combined organic layers were washed with brine and dried over MgSO₄. The solvent was removed by evaporation and the crude product was purified by flash chromatography on silica gel eluting with EtOAc/hexanes to give a mixture of (2-tert-butyldimethylsiloxyphenyl)((R)—N-Boc-piperidin-3-yl)methanone and (2-hydroxyphenyl)((R)—N-Boc-piperidin-3-yl)methanone. A −20° C. solution of the crude mixture in tetrahydrofuran was treated with 1.3 M 4-methoxybutylmagensium chloride in THF (14.9 mL, 19.4 mmol). The reaction was stirred and allowed to warm to rt over a two hour period. The reaction was quenched with ammonium chloride. The aq layer was extracted with Et₂O. The combined organic layers were dried over MgSO₄ and filtered. The solvent was evaporated and the crude product was purified by flash chromatography on silica gel eluting with EtOAc/hexanes to afford 2-((S)-1-hydroxy-5-methoxy-1-((R)—N-Boc-piperidin-3-yl)pentyl)[tert-butyldimethylsiloxy]benzene (874 mg, 47%) and 2-((S)-1-hydroxy-5-methoxy-1-((R)—N-Boc-piperidin-3-yl)pentyl)phenol (650 mg, 45%).

To a solution of 2-((S)-1-hydroxy-5-methoxy-1-((R)—N-Boc-piperidin-3-yl)pentyl)[tert-butyldimethylsiloxy]benzene (710 mg, 1.40 mmol) in tetrahydrofuran (7 mL) was added 1M tetrabutylammonium fluoride in THF (2.1 mL, 2.1 mmol). The mixture was stirred at rt for 1 h. The mixture was diluted with EtOAc (20 mL) and washed with brine twice. The organic layer was dried over sodium sulfate and filtered. The filtrate was evaporated to give a residue, which was purified by flash chromatography on silica gel eluting with EtOAc/hexanes to give 2-((S)-1-hydroxy-5-methoxy-1-((R)—N-Boc-piperidin-3-yl)pentyl)[tert-butyldimethylsiloxy]benzene (450 mg, 81%).

### Step 3. ((S)-5-methoxy-1-(2-(2,2-(dimethyl)propoxy)phenyl)-1-((R)-piperidin-3-yl)pentan-1-ol hydrochloride

A solution of 2-((S)-1-hydroxy-5-methoxy-1-((R)—N-Boc-piperidin-3-yl)pentyl)phenol (195 mg, 0.500 mmol), 1-bromo-2,2-dimethylpropane (1.0 ml, 7.5 mmol), and cesium carbonate (230 mg, 0.71) in NMP (2 mL) was heated and stirred in a microwave reactor for 20 min at 130° C. After removal of solvent, the mixture was redissolved in methylene chloride and filtered. The filtrate was evaporated to give a residue which was used without any further purification.

A solution of crude (R)-tert-butyl-3-((S)-1-hydroxy-5-methoxy-1-(2-(2,2-(dimethyl)propoxy)phenyl)pentyl)piperidine-1-carboxylate in MeCN (50 mL) was treated with 2M aq hydrochloric acid (50 mL) and stirred at rt overnight. The solvent was evaporated to afford ((S)-5-methoxy-1-(2-(2,2-(dimethyl)propoxy)phenyl)-1-((R)-piperidin-3-yl)pentan-1-ol hydrochloride (122 mg, 67%) as an oil.

The following piperidines were prepared using these procedures, replacing 1-bromo-2,2-dimethylpropane in Step 3 with the alkylating agent indicated and using DMF as solvent at rt in place of NMP at elevated temperature:

| Piperidine | Alkyl halide |
|---|---|
| 1-(2-(cyclopentylmethoxy)phenyl)-5-methoxy-1-((R)- | bromomethylcyclopentane |
| piperidin-3-yl)pentan-1-ol | |
| 1-(2-(cyclopentyloxy)phenyl)-5-methoxy-1-((R)-piperidin- | bromocyclopentane |
| 3-yl)pentan-1-ol | |
| 1-(2-(cyclobutylmethoxy)phenyl)-5-methoxy-1-((R)- | bromomethylcyclobutane |
| piperidin-3-yl)pentan-1-ol | |
| 1-(2-(cyclopropylmethoxy)phenyl)-5-methoxy-1-((R)- | bromomethylcyclopropane |
| piperidin-3-yl)pentan-1-ol | |
| 1-(2-(2-cyclopropylethoxy)phenyl)-5-methoxy-1-((R)- | (2- |
| piperidin-3-yl)pentan-1-ol | bromoethyl)cyclopropane |
| 1-(2-(benzyloxy)phenyl)-5-methoxy-1-((R)-piperidin-3- | benzyl bromide |
| yl)pentan-1-ol | |
| 1-(2-(4-fluorobenzyloxy)phenyl)-5-methoxy-1-((R)- | 4-fluorobenzyl bromide |
| piperidin-3-yl)pentan-1-ol | |
| 1-(2-(cyclohexylmethoxy)phenyl)-5-methoxy-1-((R)- | bromomethylcyclohexane |
| piperidin-3-yl)pentan-1-ol | |

### Preparation 7

### Piperidines from Weinreb Amides and Halodiphenylethers

### (S)-1-(2-(3-Fluorophenoxy)phenyl)-5-methoxy-1-((R)-piperidin-3-yl)pentan-1-ol

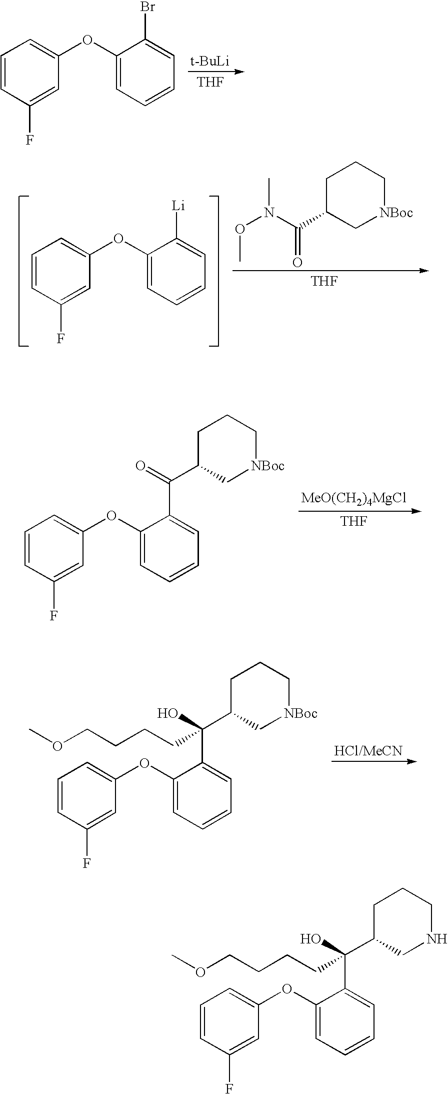

### Step 1. 2-(3-Fluorophenoxy)phenyllithium

To a stirred solution of 1-(3-fluorophenoxy)-2-bromobenzene (1.27 g, 4.75 mmol) in THF (10 mL) at −70° C. was added 1.7 M t-BuLi in pentane (5.6 mL, 9.50 mmol) dropwise to keep the temperature below −70° C. The resulting solution was stirred at −70° C. for 30 min, and used for the next step directly.

### Step 2. (3R)-1-(tert-butoxycarbonyl)-3-((3-fluorophenoxy)benzoyl)piperidine

To a solution of (R)-tert-butyl 3-(N-methoxy-N-methylcarbamoyl)piperidine-1-carboxylate (0.65 g, 2.37 mmol) in THF (4 mL) at −20° C. was added dropwise the solution of 2-(3-fluorophenoxy)phenyllithium prepared in Step 2 above. After the addition was complete, the resulting solution was allowed to warm to rt slowly, and left at rt for 1 h. The reaction was quenched with 1N HCl (˜6 mL), and extracted with Et₂O (4×10 mL). The combined organic layers were washed with satd aq NaHCO₃ and brine, and dried over Na₂SO₄. Removal of the solvent left the crude ketone (1.49 g, quantitative), which was used for next step without further purification.

### Step 3. (R)-tert-Butyl 3-((S)-1-(2-(3-fluorophenoxy)phenyl)-1-hydroxy-5-methoxy pentyl)piperidine-1-carboxylate

To a solution of (3R)-1-(tert-butoxycarbonyl)-3-(3-fluorophenoxy)benzoyl)piperidine (0.95 g, 2.37 mmol) in THF (3 mL) at −20° C. was added 1.45 M 4-methoxybutyl magnesium chloride in THF (3.3 mL, 4.76 mmol) dropwise. The resulting solution was warmed to rt slowly, and the completion of reaction was confirmed by LC-MS (˜20 min). The reaction was quenched with satd aq NH₄Cl (4 mL) and extracted with Et₂O (4×5 mL). The combined organic layers were washed with water and brine, and the solvent was removed in vacuo to give a crude product which was purified by flash column chromatography to afford (R)-tert-butyl 3-((S)-1-(2-(3-fluorophenoxy)phenyl)-1-hydroxy-5-methoxypentyl)piperidine-1-carboxylate (0.50 g, 43%).

### Step 4. (S)-1-(2-(3-Fluorophenoxy)phenyl)-5-methoxy-1-((R)-piperidin-3-yl)pentan-1-ol

To a solution of (R)-tert-butyl 3-((S)-1-(2-(3-fluorophenoxy)phenyl)-1-hydroxy-5-methoxy pentyl)piperidine-1-carboxylate (0.50 g, 1.03 mmol) in MeCN (60 mL) was added 2 N aq HCl (60 mL) slowly at rt. The resulting solution was stirred at rt overnight, then basified to pH=10 with 10 N aq NaOH. The mixture was evaporated under reduced pressure to remove MeCN. The aq layer was extracted with CH₂Cl₂ (4×10 mL), and the combined organic layers were washed with brine and dried over Na₂SO₄. The solvent was removed under vacuum to give (S)-1-(2-(3-fluorophenoxy)phenyl)-5-methoxy-1-((R)-piperidin-3-yl)pentan-1-ol (0.40 g, quantitative) as a free amine.

The following piperidines prepared using the above procedures using the halodiphenyl ethers listed below in Step 1.

| Piperidine | Halodiphenyl ether |
|---|---|
| (S)-1-(2-(2-ethylphenoxy)phenyl)-5-methoxy- | 1-(2-ethylphenoxy)-2-bromobenzene |
| 1-((R)-piperidin-3-yl)pentan-1-ol | |
| (S)-1-(2-(4-fluorophenoxy)phenyl)-5-methoxy- | 1-(4-fluorophenoxy)-2-bromobenzene |
| 1-((R)-piperidin-3-yl)pentan-1-ol | |
| (S)-1-(2-(m-tolyloxy)phenyl)-5-methoxy-1- | 1-(2-bromophenoxy)-3-methylbenzene |
| ((R)-piperidin-3-yl)pentan-1-ol | |
| (S)-1-(2-(o-tolyloxy)-3-methylphenyl)-5- | 2-(o-tolyloxy)-1-bromo-3-methylbenzene |
| methoxy-1-((R)-piperidin-3-yl)pentan-1-ol | |
| (S)-1-(2-(o-tolyloxy)-3,5-difluorophenyl)-5- | 2-(o-tolyloxy)-1-bromo-3,5- |
| methoxy-1-((R)-piperidin-3-yl)pentan-1-ol | difluorobenzene |
| (S)-1-(2-(4-fluoro-2-methylphenoxy)phenyl)-5- | 1-(4-fluoro-2-methylphenoxy)-2- |
| methoxy-1-((R)-piperidin-3-yl)pentan-1-ol | bromobenzene |
| (S)-1-(2-(5-fluoro-2-methylphenoxy)phenyl)-5- | 1-(5-fluoro-2-methylphenoxy)-2- |
| methoxy-1-((R)-piperidin-3-yl)pentan-1-ol | bromobenzene |
| (S)-1-(3,5-difluoro-2-phenoxyphenyl)-5- | 2-(o-tolyloxy)-1-bromo-3,5- |
| methoxy-1-((R)-piperidin-3-yl)pentan-1-ol | difluorobenzene |
| (S)-1-(5-fluoro-2-phenoxyphenyl)-5-methoxy- | 2-bromo-4-fluoro-1-phenoxybenzene |
| 1-((R)-piperidin-3-yl)pentan-1-ol | |
| (S)-1-(3-fluoro-2-(o-tolyloxy)phenyl)-5- | 1-bromo-3-fluoro-2-(o-tolyloxy)benzene |
| methoxy-1-((R)-piperidin-3-yl)pentan-1-ol | |
| (S)-5-methoxy-1-(3-methyl-2-(o- | 1-bromo-3-methyl-2-(o-tolyloxy)benzene |
| tolyloxy)phenyl)-1-((R)-piperidin-3-yl)pentan- | |
| 1-ol | |
| (S)-1-(5-fluoro-2-(4-fluorophenoxy)phenyl)-5- | 2-bromo-4-fluoro-1-(4- |
| methoxy-1-((R)-piperidin-3-yl)pentan-1-ol | fluorophenoxy)benzene |
| (S)-1-(2-(2,6-dimethylphenoxy)phenyl)-5- | 2-(2-bromophenoxy)-1,3-dimethylbenzene |
| methoxy-1-((R)-piperidin-3-yl)pentan-1-ol | |
| (S)-1-(3-chloro-2-(o-tolyloxy)phenyl)-5- | 1-bromo-3-chloro-2-(o-tolyloxy)benzene |
| methoxy-1-((R)-piperidin-3-yl)pentan-1-ol | |
| (S)-1-(2-(2-chloro-6-methylphenoxy)phenyl)-5- | 2-(2-bromophenoxy)-1-chloro-3- |
| methoxy-1-((R)-piperidin-3-yl)pentan-1-ol | methylbenzene |
| (S)-1-(3-chloro-2-(2-ethylphenoxy)phenyl)-5- | 1-bromo-3-chloro-2-(2- |
| methoxy-1-((R)-piperidin-3-yl)pentan-1-ol | ethylphenoxy)benzene |
| (S)-1-(3-chloro-2-(3-ethylphenoxy)phenyl)-5- | 1-bromo-3-chloro-2-(3- |
| methoxy-1-((R)-piperidin-3-yl)pentan-1-ol | ethylphenoxy)benzene |

The following piperidines were prepared using the above procedures except that in Step 1 Grignard reagents were prepared from the halodiphenyl ethers listed below instead of organolithiums.

| Piperidine | Halodiphenyl ether |
|---|---|
| (S)-1-(3-fluoro-2-phenoxyphenyl)-5-methoxy-1- | 1-chloro-3-fluoro- |
| ((R)-piperidin-3-yl)pentan-1-ol | 2-phenoxybenzene |
| (S)-1-(2-(p-tolyloxy)-3-fluorophenyl)-5-methoxy- | 2-(p-tolyloxy)-1- |
| 1-((R)-piperidin-3-yl)pentan-1-ol | chloro-3- |
| | fluorobenzene |

### Preparation 8

### BOC Protected Piperidines from Weinreb Amides and Iododiphenyl Ethers

### (R)-tert-butyl 3-((S)-1-(2-(o-tolyloxy)phenyl)-1-hydroxy-5-methoxypentyl)piperidine-1-carboxylate

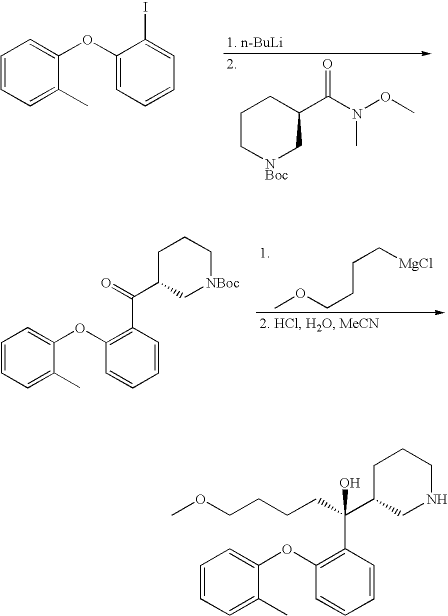

### Step 1. (2-(O-tolyloxy)phenyl)((R)-1-(tert-butoxycarbonyl)piperidin-3-yl)methanone

To a solution of 1-(o-tolyloxy)-2-iodobenzene (40 g, 0.13 mol) in anhydrous THF (500 mL) cooled to −78° C. was added dropwise 1.6 M n-BuLi in hexanes (52 mL, 0.13 mol). After stirring for 1 h at −78° C., a solution of (R)-tert-butyl 3-(N-methoxy-N-methylcarbamoyl)-piperidine-1-carboxylate (35 g, 0.13 mol) in anhydrous THF (500 mL) was added dropwise. The mixture was allowed to warm to rt and stirred overnight. Saturated aq NH₄Cl (500 mL) was added and the mixture was extracted with EtOAc (3×150 mL). The combined organic layers were dried over Na₂SO₄. Solvent removal and flash column chromatography afforded (2-(o-tolyloxy)phenyl)((R)-1-(tert-butoxycarbonyl)piperidin-3-yl)methanone (23 g, 45%).

### Step 2. (R)-tert-butyl 3-((S)-1-(2-(o-tolyloxy)phenyl)-1-hydroxy-5-methoxypentyl)piperidine-1-carboxylate

A 500-mL, three-necked flask was charged with magnesium turnings (12 g, 0.5 mol) and a small crystal of iodine. The flask was evacuated and refilled with N₂. A solution of 1-chloro-4-methoxybutane (50 g, 0.4 mol) in THF (200 mL) was added dropwise to the mixture. The reaction mixture was stirred at reflux for 2 h and most of magnesium was consumed. The solution of Grignard reagent was cooled to rt.

A 1000 mL, three-necked flask was charged with the (2-(o-tolyloxy)phenyl)((R)-1-(tert-butoxycarbonyl)piperidin-3-yl)methanone (20 g, 0.05 mol) and THF (250 mL). The flask was evacuated and refilled with N₂, the mixture was cooled with a dry ice-acetone bath and the Grignard reagent was added dropwise. The mixture was allowed to warm slowly to rt and stirred overnight. After quenching with satd aq NH₄Cl (500 mL), the mixture was extracted with EtOAc (3×150 mL) and the combined organic layers were dried over Na₂SO₄. The solvent was removed and the crude product was purified by flash column chromatography to afford the (R)-tert-butyl 3-(S)-1-(2-(o-tolyloxy)phenyl)-1-hydroxy-5-methoxypentyl)piperidine-1-carboxylate (20 g, 83%).

### Step 3. (S)-1-(2-o-tolyloxy)phenyl)-5-methoxy-1-((R)-piperidin-3-yl)pentan-1-ol

The Boc protecting group was removed using the protocol described in Preparation 6 Step 4.

The following piperidines were prepared using the above procedures from the iododiphenyl ether indicated.

| Piperidine | Iododiphenyl ether |
|---|---|
| (S)-1-(2-(2-chlorophenoxy)phenyl)-5-methoxy- | 1-(2-iodophenoxy)-2- |
| 1-((R)-piperidin-3-yl)pentan-1-ol | chlorobenzene |
| (S)-1-(2-(2-(trifluoromethyl)phenoxy)phenyl)- | 1-(2-iodophenoxy)-2- |
| 5-methoxy-1-((R)-piperidin-3-yl)pentan-1-ol | (trifluoromethyl)benzene |
| (S)-1-(2-(2-fluorophenoxy)phenyl)-5-methoxy- | 1-(2-iodophenoxy)-2- |
| 1-((R)-piperidin-3-yl)pentan-1-ol | fluorobenzene |

### Preparation 9

### Piperidines from Weinreb Amides and Bromobiaryls

### (3R)-tert-butyl 3-((S)-1-(2-(2-chlorophenyl)phenyl)-1-hydroxy-5-methoxypentyl)piperidine-1-carboxylate

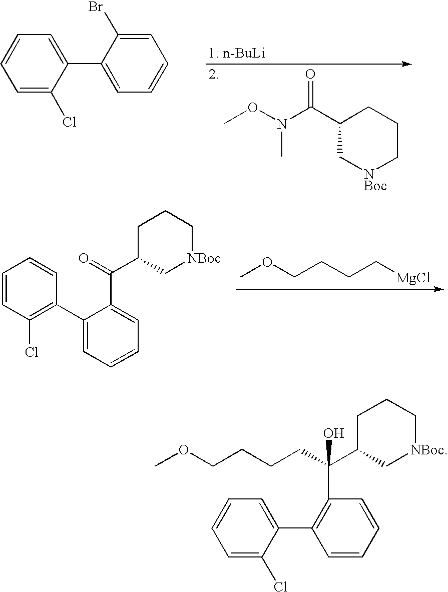

### Step 1. (3R)-1-(tert-butoxycarbonyl)-3-((2-(2-chlorophenyl))benzoyl)piperidine

To a solution of 2′-bromo-2-chloro-biphenyl (5.34 g, 20 mmol) in anhydrous THF (50 mL) cooled to −78° C. was added dropwise a solution of 1.6 M n-BuLi in hexane (12.5 mL, 20 mmol). The reaction mixture was stirred at −78° C. for 1 h and a solution of (R)-tert-butyl 3-(N-methoxy-N-methylcarbamoyl)-piperidine-1-carboxylate (5.44 g, 20 mmol) in anhydrous THF (50 mL) was added. The mixture was allowed to warm to rt and stirred overnight. The mixture was quenched with satd aq NH₄Cl (100 mL) and extracted with EtOAc (3×75 mL). The combined organic layers were dried over Na₂SO₄ and concentrated to give the crude product, which was purified by flash column chromatography to afford (3R)-1-(tert-butoxycarbonyl)-3-((2-(2-chlorophenyl))benzoyl)piperidine (4.43 g, 55%).

### Step 2. (3R)-tert-butyl 3-((S)-1-(2-(2-chlorophenyl)phenyl)-1-hydroxy-5-methoxypentyl)piperidine-1-carboxylate

A 250 mL three-necked flask was charged with magnesium turning (2.88 g, 0.12 mol) and a small crystal of iodine. The flask was evacuated and refilled with N₂. A solution of 1-chloro-4-methoxybutane (15 g, 0.12 mol) in THF (60 ml) was added dropwise to the above mixture. After heating under reflux for 2 h most of magnesium had been consumed and the Grignard solution was cooled to rt. A 250 mL three-necked flask was charged with (3R)-1-(tert-butoxycarbonyl)-3-((2-(2-chlorophenyl))benzoyl)piperidine (4.43 g, 11 mmol) and THF (50 mL), evacuated and refilled with N2. The mixture was cooled in a dry ice-acetone bath and the Grignard reagent was added dropwise. The mixture was allowed to warm slowly to rt and stirred overnight. The mixture was quenched with satd aq NH₄Cl (100 mL) and extracted with EtOAc. The combined organic layers were dried over Na₂SO₄ and concentrated to give the crude product which was purified by flash column chromatography to afford pure (3R)-tert-butyl 3-((S)-1-(2-(2-chlorophenyl)phenyl)-1-hydroxy-5-methoxypentyl)piperidine-1-carboxylate (2.5 g, 47%).

The following piperidines were prepared using procedures analogous to those described above substituting the bromobiphenyls indicated in Step 1:

| Piperidine | Bromobiphenyl |
|---|---|
| 1-(biphenyl-2-yl)-5-methoxy-1-((R)-piperidin-3- | 2-bromo-1,1′-biphenyl |
| yl)pentan-1-ol | |
| 5-methoxy-1-((R)-piperidin-3-yl)-1-(2-(pyridin-2- | 2-(2-bromophenyl)pyridine |
| yl)phenyl)pentan-1-ol | |
| (S)-5-methoxy-1-(2-(5-methylfuran-2-yl)phenyl)-1- | 2-(2-bromophenyl)-5-methylfuran |
| ((R)-piperidin-3-yl)pentan-1-ol | |
| (1S)-5-methoxy-1-(2′-methylbiphenyl-2-yl)-1-((R)- | 2-bromo-2′-methylbiphenyl |
| piperidin-3-yl)pentan-1-ol | |
| (S)-5-methoxy-1-(3′-methylbiphenyl-2-yl)-1-((R)- | 2-bromo-3′-methylbiphenyl |
| piperidin-3-yl)pentan-1-ol | |
| (S)-5-methoxy-1-(4′-methylbiphenyl-2-yl)-1-((R)- | 2-bromo-4′-methylbiphenyl |
| piperidin-3-yl)pentan-1-ol | |
| (1S)-1-(2′-fluorobiphenyl-2-yl)-5-methoxy-1-((R)- | 2-bromo-2′-fluorobiphenyl |
| piperidin-3-yl)pentan-1-ol | |
| (S)-1-(3′-fluorobiphenyl-2-yl)-5-methoxy-1-((R)- | 2-bromo-3′-fluorobiphenyl |
| piperidin-3-yl)pentan-1-ol | |
| (S)-1-(4′-fluorobiphenyl-2-yl)-5-methoxy-1-((R)- | 2-bromo-4′-fluorobiphenyl |
| piperidin-3-yl)pentan-1-ol | |
| (S)-1-(6-fluorobiphenyl-2-yl)-5-methoxy-1-((R)- | 2-bromo-6-fluorobiphenyl |
| piperidin-3-yl)pentan-1-ol | |
| (S)-1-(3′-chlorobiphenyl-2-yl)-5-methoxy-1-((R)- | 2-bromo-3′-chlorobiphenyl |
| piperidin-3-yl)pentan-1-ol | |
| (S)-1-(4′-chlorobiphenyl-2-yl)-5-methoxy-1-((R)- | 2-bromo-4′-chlorobiphenyl |
| piperidin-3-yl)pentan-1-ol | |
| (S)-1-(6-chlorobiphenyl-2-yl)-5-methoxy-1-((R)- | 2-bromo-6-chlorobiphenyl |
| piperidin-3-yl)pentan-1-ol | |
| (S)-1-(6-fluoro-3′-methylbiphenyl-2-yl)-5-methoxy-1- | 2-bromo-6-fluoro-3′-methylbiphenyl |
| ((R)-piperidin-3-yl)pentan-1-ol | |
| (1S)-1-(2′-fluoro-5′-methylbiphenyl-2-yl)-5-methoxy- | 2′-bromo-2-fluoro-5-methylbiphenyl |
| 1-((R)-piperidin-3-yl)pentan-1-ol | |
| (S)-1-(4-fluoro-3′-methylbiphenyl-2-yl)-5-methoxy-1- | 2-bromo-4-fluoro-3′-methylbiphenyl |
| ((R)-piperidin-3-yl)pentan-1-ol | |
| (S)-1-(5-fluoro-3′-methylbiphenyl-2-yl)-5-methoxy-1- | 2-bromo-5-fluoro-3′-methylbiphenyl |
| ((R)-piperidin-3-yl)pentan-1-ol | |
| (1S)-1-(3′,4′-difluorobiphenyl-2-yl)-5-methoxy-1-((R)- | 2-bromo-3′,4′-difluorobiphenyl |
| piperidin-3-yl)pentan-1-ol | |
| (S)-1-(2′,3′-difluorobiphenyl-2-yl)-5-methoxy-1-((R)- | 2′-bromo-2,3-difluorobiphenyl |
| piperidin-3-yl)pentan-1-ol | |
| (S)-1-(3′,6-difluorobiphenyl-2-yl)-5-methoxy-1-((R)- | 2-bromo-3′,6-difluorobiphenyl |
| piperidin-3-yl)pentan-1-ol | |
| (S)-1-(6-fluoro-3′,5′-dimethylbiphenyl-2-yl)-5- | 2-bromo-6-fluoro-3′,5′- |
| methoxy-1-((R)-piperidin-3-yl)pentan-1-ol | dimethylbiphenyl |
| (1S)-1-(2′,6-difluoro-5′-methylbiphenyl-2-yl)-5- | 2′-bromo-2,6′-difluoro-5- |
| methoxy-1-((R)-piperidin-3-yl)pentan-1-ol | methylbiphenyl |
| (S)-1-(6-chloro-3′-methylbiphenyl-2-yl)-5-methoxy-1- | 2-bromo-6-chloro-3′-methylbiphenyl |
| ((R)-piperidin-3-yl)pentan-1-ol | |
| (S)-1-(3′-chloro-6-fluorobiphenyl-2-yl)-5-methoxy-1- | 2-bromo-3′-chloro-6-fluorobiphenyl |
| ((R)-piperidin-3-yl)pentan-1-ol | |
| (S)-1-(6-chloro-3′-fluorobiphenyl-2-yl)-5-methoxy-1- | 2-bromo-6-chloro-3′-fluorobiphenyl |
| ((R)-piperidin-3-yl)pentan-1-ol | |
| (S)-5-methoxy-1-((R)-piperidin-3-yl)-1-(3′- | 2-bromo-3′-(trifluoromethyl)biphenyl |
| (trifluoromethyl)biphenyl-2-yl)pentan-1-ol | |
| (S)-1-(3′,6-dichlorobiphenyl-2-yl)-5-methoxy-1-((R)- | 2-bromo-3′,6-dichlorobiphenyl |
| piperidin-3-yl)pentan-1-ol | |
| (1S)-1-(3′-chloro-2′,6-difluorobiphenyl-2-yl)-5- | 2′-bromo-3-chloro-2,6′-difluorobiphenyl |
| methoxy-1-((R)-piperidin-3-yl)pentan-1-ol | |
| (S)-1-(5-bromo-3′-methylbiphenyl-2-yl)-5-methoxy- | 5-bromo-2-iodo-3′-methylbiphenyl |
| 1-((R)-piperidin-3-yl)pentan-1-ol | |
| (S)-1-(6-fluoro-3′-methoxy-5′-methylbiphenyl-2-yl)- | 2-bromo-6-fluoro-3′-methoxy-5′- |
| 5-methoxy-1-((R)-piperidin-3-yl)pentan-1-ol | methylbiphenyl |
| (S)-1-(6-chloro-3′-ethylbiphenyl-2-yl)-5-methoxy-1- | 2-bromo-6-chloro-3′-ethylbiphenyl |
| ((R)-piperidin-3-yl)pentan-1-ol | |
| (S)-1-(3-chloro-2-(2-methylbenzyl)phenyl)-5- | 1-bromo-3-chloro-2-(2- |
| methoxy-1-((R)-piperidin-3-yl)pentan-1-ol | methylbenzyl)benzene |
| (S)-1-(3-chloro-2-(3-methylbenzyl)phenyl)-5- | 1-bromo-3-chloro-2-(3- |
| methoxy-1-((R)-piperidin-3-yl)pentan-1-ol | methylbenzyl)benzene |
| (S)-1-(3′-ethyl-6-fluorobiphenyl-2-yl)-5-methoxy-1- | 2-bromo-3′-ethyl-6-fluorobiphenyl |
| ((R)-piperidin-3-yl)pentan-1-ol | |
| (S)-1-(6-chloro-3′-ethylbiphenyl-2-yl)-5-methoxy-1- | 2-bromo-6-chloro-3′-ethylbiphenyl |
| ((R)-piperidin-3-yl)pentan-1-ol | |
| (S)-1-(3-chloro-2-(quinolin-3-yl)phenyl)-5-methoxy- | 3-(2-bromo-6-chlorophenyl)quinoline |
| 1-((R)-piperidin-3-yl)pentan-1-ol | |
| (S)-1-(3′-ethoxy-6-fluoro-5′- | 2-bromo-3′-ethoxy-6-fluoro-5′- |
| (trifluoromethyl)biphenyl-2-yl)-5-methoxy-1-((R)- | (trifluoromethyl)biphenyl |
| piperidin-3-yl)pentan-1-ol | |

### Preparation 10

### Piperidine from Weinreb Amide and Metallated Fluorodiphenyl Ether

### (S)-1-(3-(o-tolyloxy)-2-fluorophenyl)-5-methoxy-1-((R)-piperidin-3-yl) pentan-1-ol

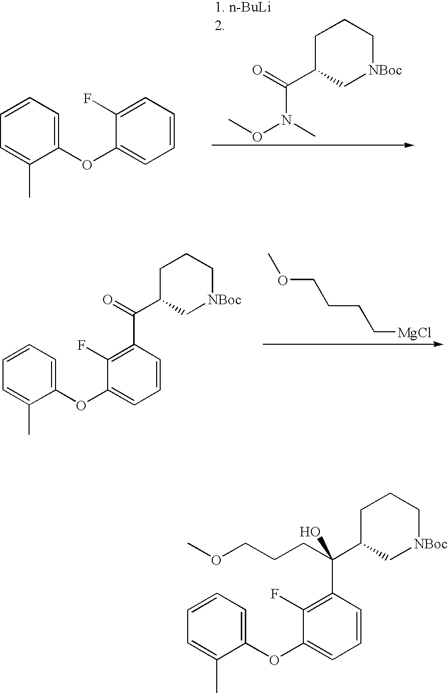

### Step 1. (3R)-1-tert-butoxycarbonyl-3-(2-fluoro-3-o-tolyloxy)benzoyl)piperidine

A solution of 2.0 mL of 2.0 M n-BuLi (2.0 mL, 4.0 mmol) was added dropwise to a solution of 1-(o-tolyloxy)-2-fluorobenzene (0.7009 g, 3.5 mmol) in THF (15 mL); the internal temperature was maintained below −70° C. during the addition. A pale, yellow slurry resulted. Confirmation of proton abstraction was confirmed by quenching an aliquot on solid 12. A solution of (R)-tert-butyl 3-(N-methoxy-N-methylcarbamoyl)piperidine-1-carboxylate (1.1159 g, 4.1 mmol) in THF (15 mL) was added dropwise. The reaction was permitted to warm to rt and stirred at for 12 h. The reaction was quenched at 0° C. with satd aq NH₄Cl and extracted with Et₂O. The Et₂O extracts were washed with aq NH₄Cl and brine and dried over Na₂SO₄. Removal of the solvent left crude (3R)-1-tert-butoxycarbonyl-3-(2-fluoro-3-o-tolyloxy)-benzoyl)piperidine (1.79 g, ˜80% pure, quantitative) which was used directly without further purification.

### Step 2. (R)-tert-butyl 3-(S)-1-(3-(o-tolyloxy)-2-fluorophenyl)-1-hydroxy-5-methoxypentyl)piperidine-1-carboxylate

A solution of crude (3R)-1-tert-butoxycarbonyl-3-(2-fluoro-3-(o-tolyloxy)benzoyl)piperidine (1.79 g, ˜80% pure, 3.5 mmol) in THF (15 mL) was cooled to 0° C. A 1.63M solution of 4-methoxybutylmagnesium chloride in THF was added with fast dropwise addition. The reaction was stirred for 1 h at rt, cooled to 0° C. and then quenched with satd aq NH₄Cl. The crude mixture was taken up into Et₂O, washed with satd aq NH₄Cl and brine, and dried over Na₂SO₄. Removal of the solvent gave an oil (1.82 g). Flash chromatography on a 40-g silica cartridge eluting with a gradient from 0 to 100% EtOAc in hexanes. Appropriate fractions were combined and stripped to give (R)-tert-butyl 3-((S)-1-(3-(o-tolyloxy)-2-fluorophenyl)-1-hydroxy-5-methoxypentyl)piperidine-1-carboxylate (0.66 g, 30%).

### Preparation 11

### (3R,4S)-3-(tert-butoxycarbonylamino)-4-(4-(tert-butyldimethylsilyloxy)pyrrolidine

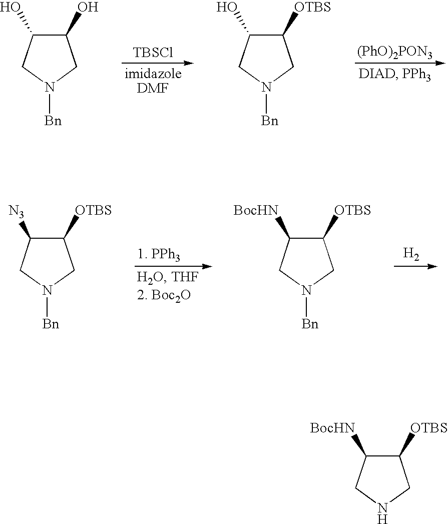

### Step 1. (3S,4S)-1-benzyl-3-hydroxy-4-(tert-butyldimethylsilyloxy)pyrrolidine

To a stirred solution of (3S,4S)-1-benzyl-3,4-dihydroxypyrrolidine (1.00 g, 5.2 mmol) and imidazole (0.71 g, 10.4 mmol) in DMF (10 mL) was added t-butyldimethylsilyl chloride (0.47 g, 3.1 mmol). The solution was stirred overnight at rt, diluted with Et₂O (80 mL) and washed with water (2×35 mL). The combined water washes were back extracted with Et₂O (30 mL). The combined Et₂O layers were washed with brine (10 mL), dried over MgSO₄ and concentrated to leave an oil (0.85 g). The crude product was applied to a 12-g silica cartridge and eluted with a 0-100% EtOAc in hexanes gradient to afford (3S,4S)-1-benzyl-3-hydroxy-4-(t-butyldimethyl-silyloxy)pyrrolidine (0.56 g, 35%)

### Step 2. (3R,4S)-1-benzyl-3-azido-4-(tert-butyldimethylsilyloxy)pyrrolidine

A stirred solution of (3S,4S)-1-benzyl-3-hydroxy-4-(t-butyldimethylsilyloxy)pyrrolidine (530 mg, 1.70 mmol), triphenylphosphine (542 mg, 2.07 mmol) and diisopropyl azodicarboxylate (407 □L, 2.07 mmol) in dry THF (30 mL) was cooled in an ice bath and diphenylphosphoryl azide (445 mL, 2.07 mmol) was added. The ice bath was allowed to melt and the mixture was stirred overnight at rt. The reaction mixture was concentrated to leave a viscous oil which was applied to a 40-g silica cartridge and eluted with a gradient from 0 to 100% EtOAc in hexanes. Fractions containing the desired product were pooled and concentrated to leave crude (3R,4S)-1-benzyl-3-azido-4-(tert-butyldimethylsilyloxy)pyrrolidine (631 mg, 110%).

### Step 3. (3R,4S)-1-benzyl-3-amino-4-(tert-butyldimethylsilyloxy)pyrrolidine

To a stirred solution of crude (3R,4S)-1-benzyl-3-azido-4-(tert-butyldimethylsilyloxy)pyrrolidine (631 mg, 1.90 mmol) in THF (18 mL) and water (2 mL) was added triphenylphosphine (562 mg, 2.15 mmol). The mixture was heated at reflux for 1 h and concentrated to leave a viscous oil. This material was taken up in Et₂O (150 mL) and extracted with 10% aq citric acid (2×50 mL). The combined aq extracts were basified by addition of solid K₂CO₃ and extracted with CH₂Cl₂ (2×100 mL). The combined CH₂Cl₂ extracts were dried over Na₂SO₄ and concentrated to leave crude (3R,4S)-1-benzyl-3-amino-4-(tert-butyldimethylsilyloxy)pyrrolidine (252 mg, 43%) as a brown oil.

### Step 4. (3R,4S)-1-benzyl-3-(tert-butoxycarbonylamino)-4-(tert-butyldimethylsilyloxy)pyrrolidine

To a stirred solution of crude (3R,4S)-1-benzyl-3-amino-4-(tert-butyldimethylsilyloxy)pyrrolidine (205 mg, 0.67 mmol) in CH₂Cl₂ (10 mL) was added di-t-butyldicarbonate (161 mg, 0.74 mmol). The mixture was stirred at rt for 20 h and concentrated to leave an oil. Flash chromatography on a 12-g silica cartridge eluted with a gradient from 0-100% EtOAc in hexanes afforded (3R,4S)-1-benzyl-3-tert-butoxycarbonylamino)-4-(tert-butyldimethyl-silyloxy)pyrrolidine (181 mg, 66%) as an oil.

### Step 5. (3R,4S)-3-(tert-butoxycarbonylamino)-4-(tert-butyldimethylsilyloxy)pyrrolidine

A solution of (3R,4S)-1-benzyl-3-tert-butoxycarbonylamino)-4-tert-butyldimethylsilyloxy)pyrrolidine (103 mg, 0.22 mmol) in MeOH (20 mL) was added to a catalytic quantity of 10% palladium hydroxide on carbon. The mixture was shaken under hydrogen gas (50 psi=0.35 MPa) for 3 h. The mixture was filtered and the filtrate was evaporated to leave (3R,4S)-3-(tert-butoxycarbonylamino)-4-(tert-butyldimethylsilyloxy)pyrrolidine (79 mg, 98%) as an oil.

### Preparation 12

### (3R*,4S*)-4-hydroxy-3-(2-(trimethylsilyl)ethoxycarbonylamino)-cyclohexanecarboxylic acid and (3R*,4S*)-3-hydroxy-4-(2-(trimethylsilyl)ethoxycarbonylamino)cyclohexanecarboxylic acid

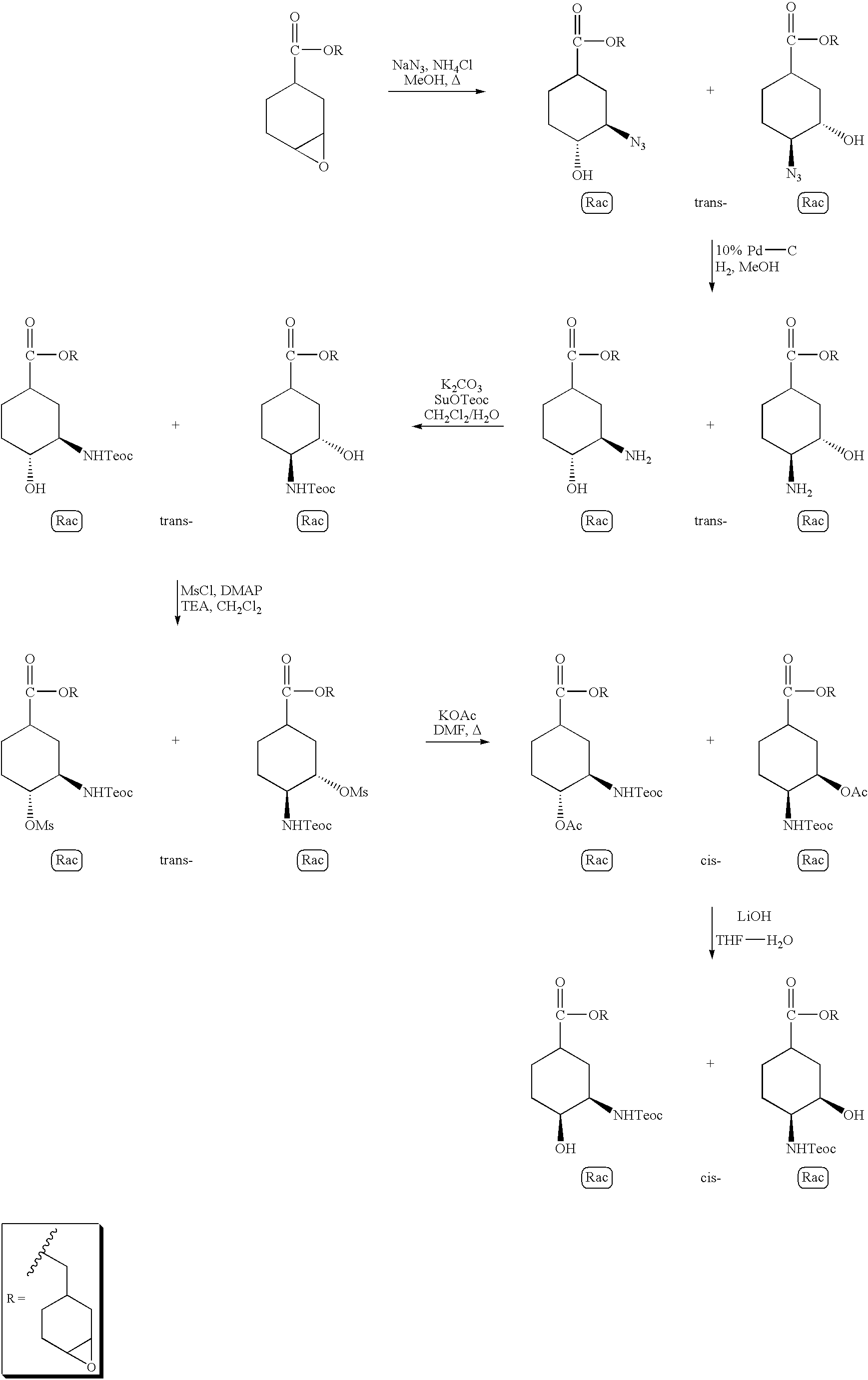

### Step 1. (3R*,4R*)-3-azido-4-hydroxycyclohexanecarboxylates and (3S*,4S*)-4-azido-3-hydroxycyclohexanecarboxylates

A mixture of 3,4-epoxycyclohexylmethyl 3,4-epoxycyclohexanecarboxylate (5.149 g, 20.4 mmol, 1.0 equiv), sodium azide (10.17 g, 156 mmol, 7.7 equiv), and ammonium chloride (8.41 g, 157 mmol, 7.7 equiv) in MeOH (60 mL) was heated at reflux for 18 h. The reaction mixture was allowed to cool to rt, the solid was filtered and the filtrate was evaporated in vacuo. The residue was combined with the solid above, dissolved in H₂O and extracted four times with CH₂Cl₂. The combined organic layers were dried over Na₂SO₄. Removal of solvent left a crude product (7.27 g) which was used in the next step without further purification.

### Step 2. (3R*,4R*)-3-amino-4-hydroxycyclohexanecarboxylates and (3S*,4S*)-4-amino-3-hydroxycyclohexanecarboxylates

To a solution of (3R*,4R*)-3-azido-4-hydroxycyclohexanecarboxylates and (3S*,4S*)-4-azido-3-hydroxycyclohexanecarboxylates (7.27 g) in MeOH was added 0.59 g of 10% Pd/C. The mixture was shaken in a Parr apparatus under 59 psi of hydrogen for 3 h. The reaction mixture was filtered to remove the catalyst and the filtrate was evaporated in vacuo. The crude product (6.27 g) was used in the next step without further purification.

### Step 3. (3R*,4R*)-4-hydroxy-3-(2-(trimethylsilyl)ethoxycarbonylamino)-cyclohexanecarboxylates and (3S*,4S*)-3-hydroxy-4-(2-(trimethylsilyl)ethoxycarbonylamino)cyclohexanecarboxylates

A mixture of (3R*,4R*)-3-amino-4-hydroxycyclohexanecarboxylates and (3S*,4S*)-4-amino-3-hydroxycyclohexanecarboxylates (6.27 g), K₂CO₃ (14.18 g, 5.0 equiv), and 1-[2-(trimethylsilyl)ethoxycarbonyloxy]-pyrrolidin-2,5-dione (12.00 g, 46.3 mmol, 2.26 equiv) in CH₂Cl₂ (150 mL) and H₂O (20 mL) was vigorously stirred at rt for 4 h. The reaction mixture was diluted with brine, extracted three times with CH₂Cl₂, dried over Na₂SO₄ and concentrated in vacuo. The crude product (7.045 g) was used in the next step without further purification.

### Step 4. (3R*,4R*)-4-methanesulfonate-3-(2-(trimethylsilyl)ethoxycarbonylamino)cyclohexanecarboxylates and (3S*,4S*)-3-methanesulfonate-4-(2-trimethylsilyl)ethoxycarbonylamino)cyclohexanecarboxylates

To a solution of (3R*,4R*)-4-hydroxy-3-(2-trimethylsilyl)ethoxycarbonylamino)cyclohexanecarboxylates and (3S*,4S*)-3-hydroxy-4-(2-(trimethylsilyl)ethoxycarbonylamino)cyclohexanecarboxylates (7.045 g, 12.2 mmol, 1.0 equiv), obtained as described above, 4-dimethylaminopyridine (0.619 g, 5.07 mmol, 0.4 equiv), and Et₃N (9.37 g, 92.6 mmol, 7.5 equiv) in CH₂Cl₂ (80 mL) was added slowly a solution of MsCl (4.52 g, 39.5 mmol, 3.2 equiv) in CH₂Cl₂ (20 mL) at 0° C. The reaction mixture was allowed to warm to rt and stirred for 67 h. The mixture was diluted with CH₂Cl₂, washed with 1N aq HCl (200 mL×1, 50 mL×1) and 10% aq Na₂CO₃, and dried over Na₂SO₄. The crude product (8.27 g, 92%) was used in the next step without further purification.

### Step 5. (3R*,4S*)-4-acetate-3-(2-(trimethylsilyl)ethoxycarbonylamino)-cyclohexanecarboxylates and (3R*,4S*)-3-acetate-4-(2-(trimethylsilyl)ethoxycarbonylamino)cyclohexanecarboxylates

A mixture of (3R*,4R*)-4-methanesulfonate-3-(2-(trimethylsilyl)ethoxycarbonylamino)cyclohexanecarboxylates and (3S*,4S*)-3-methanesulfonate-4-(2-(trimethylsilyl)ethoxycarbonylamino)cyclohexanecarboxylates (8.27 g, 11.3 mmol, 1.0 equiv) and KOAc (12.08 g, 123 mmol, 10.88 equiv) in DMF (80 mL) was heated at 100° C. for 27 h. After the solvent was removed in vacuo, the residue was dissolved in EtOAc, washed with H₂O and brine (2×), and dried over Na₂SO₄. The crude product (5.74 g, 77%) was used in the next step without further purification.

### Step 6. (3R*,4S*)-4-hydroxy-3-(2-(trimethylsilyl)ethoxycarbonylamino)cyclohexanecarboxylic acid and (3R*,4S*)-3-hydroxy-4-(2-(trimethylsilyl)ethoxycarbonylamino)cyclohexanecarboxylic acid

A mixture of (3R*,4S*)-4-acetate-3-(2-trimethylsilyl)ethoxycarbonylamino)cyclohexanecarboxylates and (3R*,4S*)-3-acetate-4-(2-(trimethylsilyl)ethoxycarbonylamino)cyclohexanecarboxylates (5.74 g, 87 mmol, 1.0 equiv), lithium hydroxide monohydrate (9.30 g, 25 equiv) in THF (200 mL) and H₂O (40 mL) was vigorously stirred at rt for 20 h. After the organic solvent was removed in vacuo, 1 N aq NaOH was added to the aq residue and the mixture was extracted three times with CH₂Cl₂. The aq phase was treated with 2 N aq HCl and extracted three times with CH₂Cl₂. These CH₂Cl₂ extracts were combined and dried over Na₂SO₄. The crude product (1.30 g) was purified by reversed-phase HPLC (Phenomenex® Luna 5μ C18(2) 100 A, 250×21.20 mm, 5 micron, 10%→90% CH₃CN/H₂O, 0.1% CF₃COOH over 13 min, flow rate 25 mL/min) to give (3R*,4S*)-4-hydroxy-3-(2-(trimethylsilyl)ethoxy-carbonylamino)cyclohexanecarboxylic acid (0.0380 g) and (3R*,4S*)-3-hydroxy-4-(2-(trimethylsilyl)ethoxycarbonylamino)cyclohexanecarboxylic acid (0.1168 g).

### Preparation 13

### Ester Hydrolysis

### (1S,3S,4R)-3-hydroxy-4-(tert-butoxycarbonylamino)cyclopentane-1-carboxylic acid

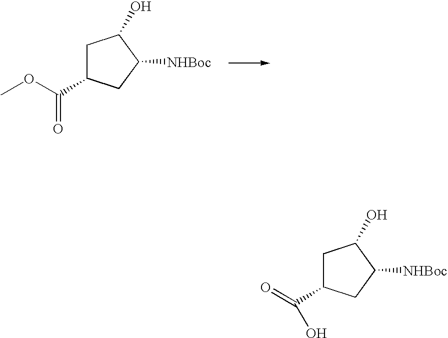

To a solution of tert-butyl (1R,2S,4S)-4-(methoxycarbonyl)-2-hydroxycyclopentylcarbamate (115 mg, 0.444 mmol) in THF (1 mL) and ethanol (1 mL), was added 1M aq NaOH solution (1 mL). The mixture was stirred for 1 h. The solvent was evaporated and the filtrate was redissolved in water. The solution was neutralized with 1M aq HCl and extracted with EtOAc. The organic layer was washed with brine and dried over sodium sulfate. The solvent was removed by evaporation and to afford tert-butyl (1S,3R,4S)-3-(tert-butoxycarbonylamino)-4-hydroxycyclopentanecarboxylic acid (94 mg, 87%).

(1S,3R,4R)-3-(tert-butoxycarbonylamino)-4-hydroxycyclopentanecarboxylic acid was prepared from (1R,2R,4S)—N—BOC-1-amino-2-hydroxycyclopentane-4-carboxylic acid methyl ester using the above procedure.

### Preparation 14

### Biaryl Syntheses

### a) 6-Bromo-2-fluoro-3′-methylbiphenyl

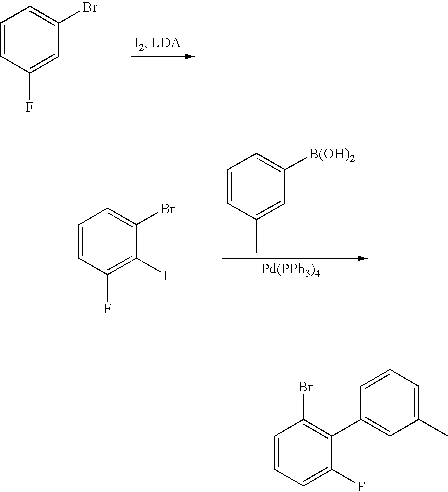

### Step 1. 1-Bromo-3-fluoro-2-iodobenzene

To a solution of diisopropylamine (76 mL, 0.4 mol) in dry THF (664 mL) and n-hexane (220 mL) was added 2.5 M n-BuLi (160 mL. 0.4 mol) dropwise at −78° C. during a period of 1 h. The mixture was stirred for 1 h at −78° C. Then a solution of 1-bromo-3-fluoro-benzene (69 g, 0.4 mol) in dry THF (300 mL) at −78° C. was added to the above mixture dropwise. After stirring for an additional 1 h at −78° C., the mixture was added a solution of iodine (101 g, 0.4 mol) in dry THF (400 mL) dropwise at −78° C. The temperature was raised from −78° C. to rt during 2 h. After stirring for 18 h at rt, the mixture was concentrated in vacuo to give crude product (120 g) which was distilled under reduced pressure to afford 1-bromo-3-fluoro-2-iodobenzene (110 g). ¹H NMR (400 MHz, DMSO): 7.24-7.19 (t, 1H), 7.38-7.32 (m, 1H), 7.55-7.53 (d, 1H).

### Step 2. 6-Bromo-2-fluoro-3′-methylbiphenyl

Pd(Ph₃P)₄ in a 500-mL round-bottom flask under N₂ atmosphere was treated sequentially with a solution of 1-bromo-3-fluoro-2-iodo-benzene (30 g, 0.1 mol) in toluene (250 mL), a solution of 2N aq Na₂CO₃ (200 mL) and 3-methyl phenylboronic acid in ethanol (62 mL). This mixture was heated at reflux under N₂ for 12 h, then cooled to rt. The mixture was partitioned between water and EtOAc. The combined organic layers were washed with brine, dried over MgSO₄, evaporated and purified by column chromatography to give 6-bromo-2-fluoro-3′-methyl-biphenyl (12 g). ¹H NMR (400 MHz, CD₃OD): 7.03 (m, 2H), 7.48-7.04 (m, 4H), 7.50 (d, 1H).

### b) 6-Bromo-2-chloro-3′-methyl-biphenyl

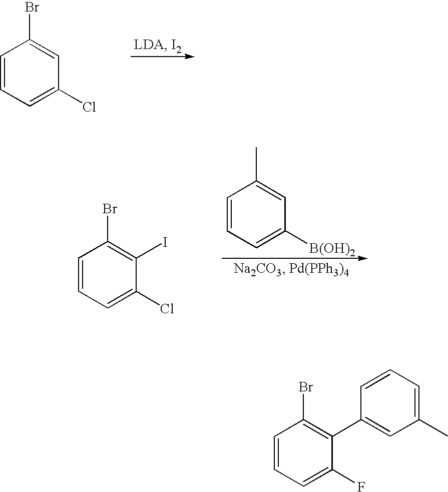

### Step 1. 1-bromo-3-chloro-2-iodobenzene

To a solution of diisopropylamine (76 mL, 0.4 mol) in anhydrous THF (664 mL) and n-hexane (220 mL) was added 2.5 M n-BuLi (160 mL, 0.4 mol) dropwise at −78° C. over 1 h. The mixture was stirred for 1 h at −78° C. and a solution of 1-bromo-3-chlorobenzene (76 g, 0.4 mol) in anhydrous THF (300 mL) was added dropwise at −78° C. After stirring for an additional 1 h at the same temperature, a solution of iodine (101 g, 0.4 mol) in anhydrous THF (400 mL) was added dropwise at −78° C. The temperature was raised from −78° C. to rt during 2 h. After stirring for 18 h at rt, the mixture was concentrated in vacuo to give the crude product (120 g) which was distilled under reduced pressure to give 1-bromo-3-fluoro-2-iodobenzene (115 g, 91%). ¹H NMR (400 MHz, CDCl₃): 7.12-7.18 (t, 1H), 7.35-7.41 (dd, 1H), 7.49-7.54 (dd, 1H); MS (E/Z): 317 (M+H⁺)

### Step 2. 6-bromo-2-chloro-3′-methyl-biphenyl

A 500-mL round-bottom flask under N₂ atmosphere was charged sequentially with Pd(Ph₃P)₄, 1-bromo-3-fluoro-2-iodobenzene (10 g, 0.032 mol) in toluene (80 mL), 2N aqueous sodium carbonate (55 mL) and 3-methylphenylboronic acid (5.16 g, 0.032 mol) dissolved in ethanol (40 mL). This mixture was heated at reflux under N₂ for 12 h and cooled to rt. The mixture was partitioned between water and EtOAc. The combined organic layers were washed with brine, dried over MgSO₄, and concentrated. The residue was purified by column chromatography to give 6-bromo-2-chloro-3′-methyl-biphenyl (6 g, 67%). ¹H NMR (400 MHz, CD₃OD): 6.90-7.00 (t, 2H), 7.14-7.24 (m, 2H), 7.26-7.33 (t, 1H), 7.44-7.50 (d, 1H), 7.58-7.62 (d, 1H); MS (E/Z): 281 (M+H⁺)

The following biaryls were prepared from aryl halides and the boronic acids indicated using the procedures described in Preparations 14a Step 2 and 14b Step 2:

| Biaryl | Aryl halide | Boronic acid |
|---|---|---|
| 2-bromobiphenyl | 2-bromoiodobenzene | phenylboronic acid |
| 2-bromo-2′-methylbiphenyl | 2-bromoiodobenzene | 2-methylphenylboronic acid |
| 2-bromo-3′-methylbiphenyl | 2-bromoiodobenzene | 3-methylphenylboronic acid |
| 2-bromo-4′-methylbiphenyl | 2-bromoiodobenzene | 4-methylphenylboronic acid |
| 2-bromo-2′-fluorobiphenyl | 2-bromoiodobenzene | 2-fluorophenylboronic acid |
| 2-bromo-3′-fluorobiphenyl | 2-bromoiodobenzene | 3-fluorophenylboronic acid |
| 2-bromo-4′-fluorobiphenyl | 2-bromoiodobenzene | 4-fluorophenylboronic acid |
| 2-bromo-2′-chlorobiphenyl | 2-bromoiodobenzene | 2-chlorophenylboronic acid |
| 2-bromo-3′-chlorobiphenyl | 2-bromoiodobenzene | 3-chlorophenylboronic acid |
| 2-bromo-4′-chlorobiphenyl | 2-bromoiodobenzene | 4-chlorophenylboronic acid |
| 2′-bromo-2-fluoro-5- | 2-bromoiodobenzene | 2-fluoro-5-methylphenylboronic |
| methylbiphenyl | | acid |
| 2-bromo-3′,4′-difluorobiphenyl | 2-bromoiodobenzene | 3,4-difluorophenylboronic acid |
| 2-bromo-3′- | 2-bromoiodobenzene | 3-(trifluoromethyl)phenylboronic |
| (trifluoromethyl)biphenyl | | acid |
| 2-bromo-6-fluorobiphenyl | 1-bromo-3-fluoro-2- | phenylboronic acid |
| | iodobenzene | |
| 2-bromo-3′-chloro-6- | 1-bromo-3-fluoro-2- | 3-chlorophenylboronic acid |
| fluorobiphenyl | iodobenzene | |
| 2-bromo-6-fluoro-3′,5′- | 1-bromo-3-fluoro-2- | 3,5-dimethylphenylboronic acid |
| dimethylbiphenyl | iodobenzene | |
| 2-bromo-3′,6-difluorobiphenyl | 1-bromo-3-fluoro-2- | 3-fluorophenylboronic acid |
| | iodobenzene | |
| 2′-bromo-2,6′-difluoro-5- | 1-bromo-3-fluoro-2- | 2-fluoro-5-methylphenylboronic |
| methylbiphenyl | iodobenzene | acid |
| 2-bromo-6-chlorobiphenyl | 1-bromo-3-chloro-2- | phenylboronic acid |
| | iodobenzene | |
| 2′-bromo-6′-chloro-2-fluoro-5- | 1-bromo-3-chloro-2- | 2-fluoro-5-methylphenylboronic |
| methylbiphenyl | iodobenzene | acid |
| 2-bromo-6-chloro-3′- | 1-bromo-3-chloro-2- | 3-methylphenylboronic acid |
| methylbiphenyl | iodobenzene | |
| 2-bromo-3′,6-dichlorobiphenyl | 1-bromo-3-chloro-2- | 3-chlorophenylboronic acid |
| | iodobenzene | |
| 2-bromo-6-chloro-3′- | 1-bromo-3-chloro-2- | 3-fluorophenylboronic acid |
| fluorobiphenyl | iodobenzene | |
| 2-(2-bromophenyl)pyridine | 2-bromopyridine | 2-bromobenzeneboronic acid |
| 2-bromo-5-fluoro-3′- | 1-bromo-4-fluoro-2- | 3-methylphenylboronic acid |
| methylbiphenyl | iodobenzene | |
| 2-bromo-6-chloro-3′- | 1-bromo-3-chloro-2- | 3-ethylphenylboronic acid |
| ethylbiphenyl | iodobenzene | |
| 2-bromo-3′-ethyl-6- | 1-bromo-3-fluoro-2- | 3-ethylphenylboronic acid |
| fluorobiphenyl | iodobenzene | |
| 2-bromo-6-chloro-3′- | 1-bromo-3-chloro-2- | 3-isopropylphenylboronic acid |
| isopropylbiphenyl | iodobenzene | |
| 2-bromo-4′,6-difluoro-3′- | 1-bromo-3-fluoro-2- | (4-fluoro-3-methylphenyl)boronic |
| methylbiphenyl | iodobenzene | acid |
| 2-bromo-6-fluoro-4′-fluoro-3′- | 1-bromo-3-fluoro-2- | (4-fluoro-3-methylphenyl)boronic |
| methylbiphenyl | iodobenzene | acid |
| 2-bromo-6-chloro-3′,5′-bis | 1-bromo-3-chloro-2- | [3,5-bis (methoxy) phenyl]boronic |
| (methoxy) biphenyl | iodobenzene | acid |
| 2-bromo-6-fluoro-3′,5′-bis | 1-bromo-3-fluoro-2- | [3,5-bis (methoxy) phenyl]boronic |
| (methoxy) biphenyl | iodobenzene | acid |
| 2-bromo-6-chloro-3′- | 1-bromo-3-chloro-2- | [3-(methoxy)phenyl]boronic acid |
| (methoxy)biphenyl | iodobenzene | |
| 2-bromo-6-fluoro-3′- | 1-bromo-3-fluoro-2- | [3-(methoxy)phenyl]boronic acid |
| (methoxy)biphenyl | iodobenzene | |
| 2-bromo-6-fluoro-3′-methyl-5′- | 1-bromo-3-fluoro-2- | [3-methyl-5- |
| (methoxy)biphenyl | iodobenzene | (methoxy)phenyl]boronic acid |
| 2-bromo-3′-(ethyloxy)-6- | 1-bromo-3-fluoro-2- | [3-(ethyloxy)-5- |
| fluoro-5′- | iodobenzene | (trifluoromethyl)phenyl]boronic |
| (trifluoromethyl)biphenyl | | acid |
| 3-(2-bromo-6- | 1-bromo-3-chloro-2- | 3-quinolinylboronic acid |
| chlorophenyl)quinoline | iodobenzene | |
| 2-(2-bromo-6- | 1-bromo-3-chloro-2- | 2-naphthalenylboronic acid |
| chlorophenyl)naphthalene | iodobenzene | |
| 3-(2-bromophenyl)pyridine | 1-bromo-2-iodobenzene | 3-pyridinylboronic acid |
| 3-(2-bromo-6- | 1-bromo-3-chloro-2- | 3-pyridinylboronic acid |
| chlorophenyl)pyridine | iodobenzene | |
| 4-(2-bromophenyl)pyridine | 1-bromo-2-iodobenzene | 4-pyridinylboronic acid |
| 4-(2-bromo-6- | 1-bromo-3-chloro-2- | 4-isoquinolinylboronic acid |
| chlorophenyl)isoquinoline | iodobenzene | |
| 2-bromo-6-fluoro-2′-fluoro-5′- | 1-bromo-3-fluoro-2- | (2-fluoro-5-methylphenyl)boronic |
| methylbiphenyl | iodobenzene | acid |
| 2-bromo-6-chloro-3′- | 1-bromo-3-chloro-2- | {3- |
| [(methoxy)methyl]biphenyl. | iodobenzene | [(methoxy)methyl]phenyl}boronic |
| | | acid |

### Preparation 15

### Morpholine Synthesis

### (R)-1-(6-Fluoro-3′-methylbiphenyl-2-yl)-5-methoxy-1-((R)-morpholin-2-yl)pentan-1-ol

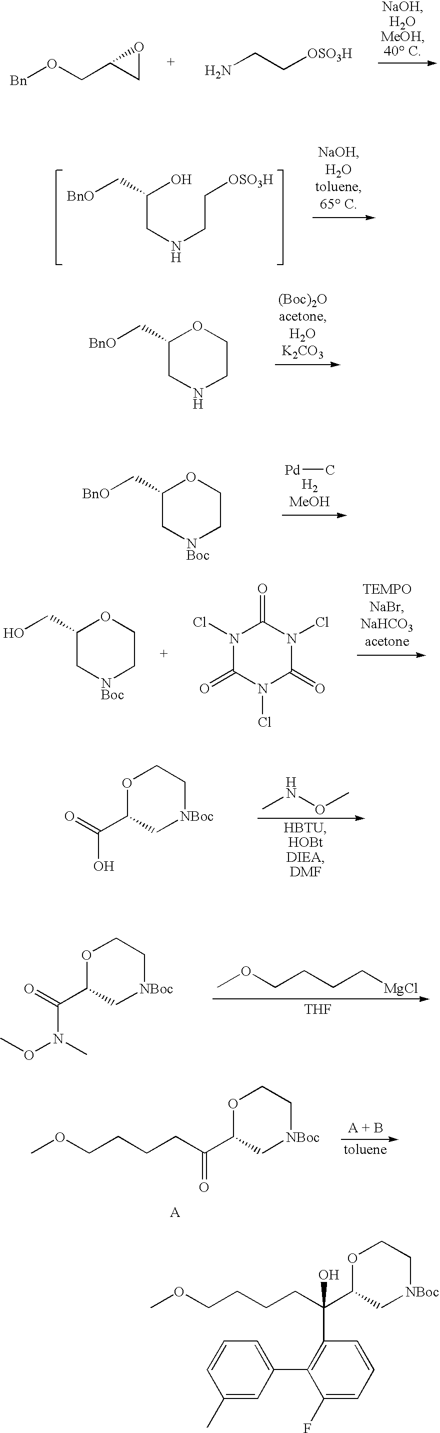

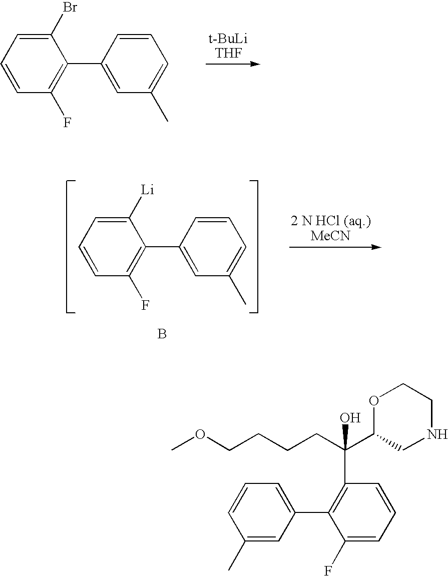

### Step 1. (R)-2-(Benzyloxymethyl)morpholine

To a stirred mixture of (R)-2-(benzyloxymethyl)oxirane (10.0 g, 60.9 mmol) and NaOH (19.49 g, 487.2 mmol) in H₂O (46 mL) and MeOH (18 mL), there was added 2-aminoethyl hydrogen sulfate (36.8 g, 255.8 mmol) in portions. After addition was complete, the reaction mixture was stirred at 40° C. for 2 h. After cooling, the mixture was treated with NaOH (15.0 g, 375.0 mmol), followed by toluene (70 mL), and stirred at 65° C. overnight. The mixture was cooled, diluted with toluene (27 mL) and H₂O (92 mL). The toluene layer was separated and the aqueous layer was extracted with CH₂Cl₂ (2×50 mL). The combined organic layers were concentrated to give crude (R)-2-benzyloxymethyl)morpholine (˜14 g), which was used without purification. MS m/z 208 (M+H⁺).

### Step 2. (R)-tert-Butyl 2-(benzyloxymethyl)morpholine-4-carboxylate

To a solution of crude (R)-2-(benzyloxymethyl)morpholine (˜14 g) in acetone (100 mL) and H₂O (30 mL) at 0° C., there was added K₂CO₃ (25.2 g, 182.7 mmol), followed by (Boc)₂O (14.6 g, 67.0 mmol). The resulting solution was warmed to rt, and stirred until no starting material remained (˜30 min). Acetone was removed under vacuum, and the aqueous solution was extracted with CH₂Cl₂ (4×10 mL). The combined organic layers were washed with H₂O (10 mL) and the solvent was removed. The residue was purified by flash column chromatography to give (R)-tert-butyl 2-(benzyloxymethyl)morpholine-4-carboxylate (8.33 g, 44% over 2 steps). ¹H NMR (400 MHz, CDCl₃): 7.34 (m, 5H), 4.56 (s, 2H), 3.88 (d, 2H), 3.82 (br, 1H), 3.40 (m, 1H), 3.48 (m, 3H), 2.94 (m, 1H), 2.76 (m, 1H), 1.44 (s, 9H); MS m/z 330 (M+Na⁺).

### Step 3. (R)-tert-Butyl 2-(hydroxymethyl)morpholine-4-carboxylate

To a solution of (R)-tert-butyl 2-(benzyloxymethyl)morpholine-4-carboxylate (8.33 g, 27.1 mmol) in EtOH was added Pd—C (wet, 3.6 g), and the resulting mixture was stirred at rt under a H₂ balloon overnight. After filtration, the solvent was removed under vacuum, and the residue was purified by flash column chromatography to give (R)-tert-butyl 2-hydroxymethyl)morpholine-4-carboxylate (5.84 g, 99%) as a clear oil. ¹H NMR (400 MHz, CDCl₃): 3.88 (d, 2H), 3.82 (br, 1H), 3.64 (d, 1H), 3.56 (m, 3H), 2.94 (m, 1H), 2.76 (m, 1H), 1.90 (br, 1H), 1.44 (s, 9H); MS m/z 218 (M+H⁺).

### Step 4. (R)-4-(tert-Butoxycarbonyl)morpholine-2-carboxylic acid

Satd aq NaHCO₃ (15 mL) was added to a solution of (R)-tert-butyl 2-(hydroxymethyl)morpholine-4-carboxylate (1.09 g, 5.0 mmol) in acetone (50 mL), stirred and maintained at 0° C.

Solid NaBr (0.1 g, 1 mmol) and TEMPO (0.015 g, 0.1 mmol) were added. Trichloroisocyanuric acid (2.32 g, 10.0 mmol) was then added slowly within 20 min at 0° C. After addition, the mixture was warmed to rt and stirred overnight. 2-Propanol (3 mL) was added, and the resulting solution was stirred at rt for 30 min, filtered through a pad of Celite, concentrated under vacuum, and treated with satd aq Na₂CO₃ (15 mL). The aqueous solution was washed with EtOAc (5 mL), acidified with 6 N HCl, and extracted with EtOAc (5×10 mL). The combined organic layers were dried over Na₂SO₄ and the solvent was removed to give (R)-4-(tert-butoxycarbonyl)morpholine-2-carboxylic acid (1.07 g, 92%) as a white solid. ¹H NMR (400 MHz, CDCl₃): 4.20 (br, 1H), 4.12 (d, 1H), 4.02 (d, 1H), 3.84 (m, 1H), 3.62 (m, 1H), 3.04 (m, 2H), 1.44 (s, 9H); MS m/z 232 (M+H⁺).

### Step 5. (R)-tert-Butyl 2-(methoxy(methyl)carbamoyl)morpholine-4-carboxylate

To a solution of (R)-4-(tert-butoxycarbonyl)morpholine-2-carboxylic acid (1.05 g, 4.54 mmol) in DMF (10 mL) at 0° C. C, was added DIEA (3.9 mL, 22.7 mmol), followed by HBTU (1.89 g, 4.99 mmol) and HOBt (0.67 g, 4.99 mmol). MeONMHMe.HCl (0.48 g, 4.92 mmol) was added and the resulting solution was warmed to rt and stirred until no starting material remained (˜2 h). The mixture was diluted with H₂O (10 mL) and extracted with EtOAc (4×10 mL). The combined organic layers were washed with 1 N aq HCl (10 mL), 1 N aq NaOH (3×10 mL), water (2×10 mL) and brine (10 mL), and dried over Na₂SO₄. The solvent was removed under vacuum to give (R)-tert-butyl 2-(methoxy(methyl)carbamoyl)morpholine-4-carboxylate (1.40 g, quant.), which was used without further purification. ¹H NMR (400 MHz, CDCl₃): 4.36 (br, 1H), 4.08 (m, 1H), 4.00 (d, 1H), 3.84 (m, 1H), 3.76 (s, 3H), 3.58 (m, 1H), 3.20 (s, 3H), 3.04 (m, 2H), 1.44 (s, 9H); MS m/z 297 (M+Na⁺).

### Step 6. (R)-tert-Butyl 2-(5-methoxypentanoyl)morpholine-4-carboxylate

To a stirred solution of (R)-tert-butyl 2-methoxy(methyl)carbamoyl)morpholine-4-carboxylate (1.37 g, 5.0 mmol) in THF (10 mL) at −20° C., there was added 1.47 M 4-methoxybutylmagnesium chloride in THF (10.2 mL, 15.0 mmol) dropwise to keep the temperature below −20° C. After addition, the resulting solution was warmed to rt and quenched with 1 N aq HCl (10 mL). The organic layer was separated, and the aqueous layer was extracted with ether (3×5 mL). Combined organic layers were washed with satd aq NaHCO₃ (10 mL) and brine (5 mL) and dried over Na₂SO₄. Removal of the solvent under vacuum gave (R)-tert-butyl 2-(5-methoxypentanoyl)morpholine-4-carboxylate (1.41 g, 93%), which was used without purification. MS m/s 324 (M+Na⁺).

### Step 7. (R)-tert-Butyl 2-((R)-1-(6-fluoro-3′-methylbiphenyl-2-yl)-1-hydroxy-5-methoxypentyl)-morpholine-4-carboxylate

To a solution of 2-bromo-6-fluoro-3′-methylbiphenyl (1.90 g, 7.17 mmol) in ether (8 mL) at −78° C., there was added t-BuLi in pentane (1.70 M, 8.43 mL, 14.33 mmol) dropwise to keep the temperature below −70° C. The resulting solution was stirred at −78° C.

To a solution of (R)-tert-butyl 2-(5-methoxypentanoyl)morpholine-4-carboxylate (0.68 g, 2.26 mmol) in toluene (8 mL) at −20° C. there was added the above lithium reagent dropwise to keep the solution temperature below −20° C. After addition, the resulting mixture was warmed to rt slowly, and quenched with saturated NH₄Cl (8 mL). The organic layer was separated, and aqueous layer was extracted with ether (3×5 mL). Combined organic layers were washed with water (10 mL), concentrated, and the residue was purified by flash column chromatography to give (R)-tert-butyl 2-((R)-1-(6-fluoro-3′-methylbiphenyl-2-yl)-1-hydroxy-5-methoxypentyl)-morpholine-4-carboxylate (0.48 g, 44%) as a foam. ¹H NMR (400 MHz, CDCl₃): 7.40 (m, 1H), 7.32 (m, 2H), 7.20 (d, 1H), 7.04 (m, 3H), 3.84 (m, 1H), 3.78 (m, 2H), 3.40-3.24 (ms, 7H), 2.82 (s, 3H), 1.70-1.20 (m, 5H), 1.44 (s, 9H), 0.94 (m, 1H); MS m/z 510 (M+Na⁺).

### Step 8. (R)-1-(6-Fluoro-3′-methylbiphenyl-2-yl)-5-methoxy-1-((R)-morpholin-2-yl)pentan-1-ol

To a solution of (R)-tert-butyl 2-((R)-1-(6-fluoro-3′-methylbiphenyl-2-yl)-1-hydroxy-5-methoxypentyl)morpholine-4-carboxylate (0.46 g, 0.96 mmol) in acetonitrile (50 mL) was added 2 N aq HCl (50 mL). The resulting solution was stirred at rt overnight and basified with 10 N aq NaOH to pH 10. Acetonitrile was removed under vacuum, and the aqueous residue was extracted with CH₂Cl₂ (4×5 mL). The combined organic layers were washed with brine (5 mL), dried over Na₂SO₄, and concentrated to give (R)-1-(6-fluoro-3′-methylbiphenyl-2-yl)-5-methoxy-1-((R)-morpholin-2-yl)pentan-1-ol (0.38, quant.). MS m/z 388 (M+H⁺).

The following morpholines were prepared using procedures analogous to those described above (R)-1-(6-chloro-3′-methylbiphenyl-2-yl)-5-methoxy-1-((R)-morpholin-2-yl)pentan-1-ol using 2-bromo-6-chloro-3′-methylbiphenyl in Step 7; (R)-1-(6-fluoro-3′-(trifluoromethoxy)biphenyl-2-yl)-5-methoxy-1-((R)-morpholin-2-yl)pentan-1-ol using 2-bromo-6-fluoro-3′-(trifluoromethoxy)biphenyl in Step 7; (R)-5-methoxy-1-(3-methoxy-3′-methylbiphenyl-2-yl)-1-((R)-morpholin-2-yl)pentan-1-ol using 2-bromo-3-methoxy-3′-methylbiphenyl in Step 7; (R)-1-3′-ethyl-6-fluorobiphenyl-2-yl)-5-methoxy-1-((R)-morpholin-2-yl)pentan-1-ol using 2-bromo-3′-ethyl-6-fluorobiphenyl in Step 7; (R)-1-(6-fluoro-3′-methoxybiphenyl-2-yl)-5-methoxy-1-((R)-morpholin-2-yl)pentan-1-ol using 2-bromo-6-fluoro-3′-methoxybiphenyl in Step 7; (R)-1-(3′-chloro-6-fluorobiphenyl-2-yl)-5-methoxy-1-((R)-morpholin-2-yl)pentan-1-ol using 2-bromo-3′-chloro-6-fluorobiphenyl in Step 7; (R)-1-(3′-cyclopropyl-6-fluorobiphenyl-2-yl)-5-methoxy-1-((R)-morpholin-2-yl)pentan-1-ol using 2-bromo-3′-cyclopropyl-6-fluorobiphenyl in Step 7; (R)-1-(6-chloro-3′-ethylbiphenyl-2-yl)-5-methoxy-1-((R)-morpholin-2-yl)pentan-1-ol using 2-bromo-6-chloro-3′-ethylbiphenyl in Step 7; (R)-1-(6-chloro-3′,4′-dimethylbiphenyl-2-yl)-5-methoxy-1-((R)-morpholin-2-yl)pentan-1-ol using 2-bromo-6-chloro-3′,4′-dimethylbiphenyl in Step 7; (R)-1-(3′-ethoxy-6-fluorobiphenyl-2-yl)-5-methoxy-1-((R)-morpholin-2-yl)pentan-1-ol using 2-bromo-3′-ethoxy-6-fluorobiphenyl in Step 7; (R)-1-(6-fluoro-3-methoxy-3′-methylbiphenyl-2-yl)-5-methoxy-1-((R)-morpholin-2-yl)pentan-1-ol using 2-bromo-6-fluoro-3-methoxy-3′-methylbiphenyl in Step 7; (R)-1-(6-chloro-3′-methoxybiphenyl-2-yl)-5-methoxy-1-((R)-morpholin-2-yl)pentan-1-ol using 2-bromo-6-chloro-3′-methoxybiphenyl in Step 7; (R)-1-(6-fluoro-3-methylthio)biphenyl-2-yl)-5-methoxy-1-((R)-morpholin-2-yl)pentan-1-ol using 2′-bromo-6′-fluoro-3-(methylthio)biphenyl in Step 7; 1-(3′,6-dichlorobiphenyl-2-yl)-5-methoxy-1-((R)-morpholin-2-yl)pentan-1-ol using 2-bromo-3′,6-dichlorobiphenyl in Step 7; (R)-1-(6-chloro-3′-isopropylbiphenyl-2-yl)-5-methoxy-1-((R)-morpholin-2-yl)pentan-1-ol using 2-bromo-6-chloro-3′-isopropylbiphenyl in Step 7; (R)-1-(6-chloro-3′-(methylthio)biphenyl-2-yl)-5-methoxy-1-((R)-morpholin-2-yl)pentan-1-ol using (2′-bromo-6′-chlorobiphenyl-3-yl)(methyl)sulfane in Step 7; (R)-1-(6-fluoro-3′-(trifluoromethyl)biphenyl-2-yl)-5-methoxy-1-((R)-morpholin-2-yl)pentan-1-ol using 2-bromo-6-fluoro-3′-trifluoromethyl)biphenyl in Step 7; (R)-5-methoxy-1-((R)-morpholin-2-yl)-1-(2-(o-tolyloxy)phenyl)pentan-1-ol using 1-(o-tolyloxy)-2-iodobenzene in Step 7; (R)-1-(4′,6-difluoro-3′-methylbiphenyl-2-yl)-5-methoxy-1-((R)-morpholin-2-yl)pentan-1-ol using 2-bromo-4′,6-difluoro-3′-methylbiphenyl in Step 7; (R)-1-(3-chloro-2-(pyridin-3-yl)phenyl)-5-methoxy-1-((R)-morpholin-2-yl)pentan-1-ol using 3-(2-bromo-6-chlorophenyl)pyridine in Step 7; (R)-1-(3-chloro-2-(3-methyl-1,2,4-oxadiazol-5-yl)phenyl)-5-methoxy-1-((R)-morpholin-2-yl)pentan-1-ol using 5-(2-bromo-6-chlorophenyl)-3-methyl-1,2,4-oxadiazole in Step 7; (R)-1-(6-fluoro-3′-methoxy-5′-methylbiphenyl-2-yl)-5-methoxy-1-((R)-morpholin-2-yl)pentan-1-ol using 2-bromo-6-fluoro-3′-methoxy-5′-methylbiphenyl in Step 7; (R)-1-(6-chloro-3′-ethylbiphenyl-2-yl)-5-methoxy-1-((R)-morpholin-2-yl)pentan-1-ol using 2-bromo-6-chloro-3′-ethylbiphenyl in Step 7; (R)-1-(6-chloro-3′-ethylbiphenyl-2-yl)-5-methoxy-1-((R)-morpholin-2-yl)pentan-1-ol using 2-bromo-6-chloro-3′-ethylbiphenyl in Step 7; (1R)-1-(6-chloro-2′-fluoro-5′-methylbiphenyl-2-yl)-5-methoxy-1-((R)-morpholin-2-yl)pentan-1-ol using 2′-bromo-6′-chloro-2-fluoro-5-methylbiphenyl in Step 7; (R)-1-(3-chloro-2-(naphthalen-2-yl)phenyl)-5-methoxy-1-((R)-morpholin-2-yl)pentan-1-ol using 2-(2-bromo-6-chlorophenyl)naphthalene in Step 7; (R)-1-(3-chloro-2-(quinolin-3-yl)phenyl)-5-methoxy-1-((R)-morpholin-2-yl)pentan-1-ol using 3-(2-bromo-6-chlorophenyl)quinoline in Step 7; (R)-1-(6-fluoro-3′,5′-dimethoxybiphenyl-2-yl)-5-methoxy-1-((R)-morpholin-2-yl)pentan-1-ol using 2-bromo-6-fluoro-3′,5′-dimethoxybiphenyl in Step 7; (R)-1-(6-chloro-3′-(methoxymethyl)biphenyl-2-yl)-5-methoxy-1-((R)-morpholin-2-yl)pentan-1-ol using 2-bromo-6-chloro-3′-(methoxymethyl)biphenyl in Step 7; (1R)-1-(3-chloro-2-(isoquinolin-4-yl)phenyl)-5-methoxy-1-((R)-morpholin-2-yl)pentan-1-ol using 4-(2-bromo-6-chlorophenyl)isoquinoline in Step 7; (R)-1-(6-chloro-3′,5′-dimethoxybiphenyl-2-yl)-5-methoxy-1-((R)-morpholin-2-yl)pentan-1-ol using 2-bromo-6-chloro-3′,5′-dimethoxybiphenyl in Step 7; (R)-1-(3′-ethoxy-6-fluoro-5′-(trifluoromethyl)biphenyl-2-yl)-5-methoxy-1-((R)-morpholin-2-yl)pentan-1-ol using 2-bromo-3′-ethoxy-6-fluoro-5′-trifluoromethyl)biphenyl in Step 7.

The following morpholines were prepared starting in Step 5 with racemic 4-(tert-butoxycarbonyl)morpholine-2-carboxylic acid:
(RS)-5-methoxy-1-((RS)-morpholin-2-yl)-1-(2-(o-tolyloxy)phenyl)pentan-1-ol
(RS)-1-(6-chloro-3′-methylbiphenyl-2-yl)-5-methoxy-1-((RS)-morpholin-2-yl)pentan-1-ol.

### Preparation 16

### tert-butyl (3R,4S)-4-(tert-butyldimethylsilyloxy)pyrrolidin-3-yl(methyl)carbamate

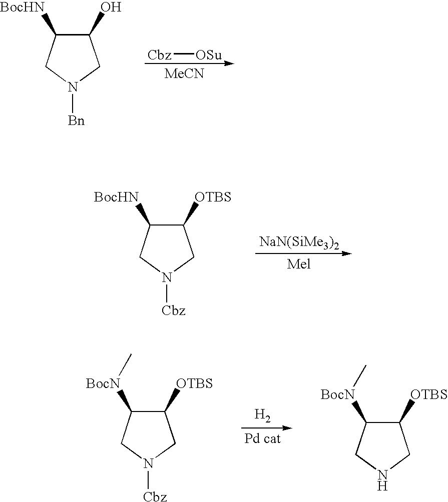

### Step 1. (3R,4S)-benzyl 3-(tert-butoxycarbonylamino)-4-(tert-butyldimethylsilyloxy)pyrrolidine-1-carboxylate

To a stirred solution of tert-butyl (3R,4S)-4-(tert-butyldimethylsilyloxy)pyrrolidin-3-ylcarbamate (320 mg, 1.01 mmol) in MeCN (10 mL) was added Cbz-OSu (380 mg, 1.52 mmol). The mixture was stirred at rt for 24 h. 10% aq K2CO3 (10 mL) was added and stirring was continued for a further 18 h. Acetonitrile was removed on the rotary evaporator and the aqueous residue was extracted with ether (100 mL). The ether layer was dried over MgSO4 and concentrated to afford an oil (450 mg) which was purified by chromatography on a 40-g silica cartridge eluted with a gradient from 0-80% EtOAc in hexanes to afford (3R,4S)-benzyl 3-(tert-butoxycarbonylamino)-4-(tert-butyldimethylsilyloxy)pyrrolidine-1-carboxylate (360 mg, 79%) as a colorless oil.

### Step 2. (3R,4S)-benzyl 3-(tert-butoxycarbonyl(methyl)amino)-4-(tert-butyldimethyl-silyloxy)pyrrolidine-1-carboxylate

A stirred solution of (3R,4S)-benzyl 3-(tert-butoxycarbonylamino)-4-tert-butyldimethylsilyloxy)pyrrolidine-1-carboxylate (140 mg, 0.31 mmol) in dry THF (2 mL) was cooled to −70° C. and 2M sodium bis(trimethylsilyl)amide in THF (0.5 mL, 1.0 mmol) was added dropwise over 2 min. The mixture was stirred at −70° C. for 10 min and methyl iodide (0.2 mL, 3.1 mmol) was added. The cooling bath was allowed to expire and the mixture was stirred at for 3 h as it warmed to rt. The mixture was diluted with ether (90 mL), washed with satd aq NaHCO3 (20 mL) and brine (20 mL) and dried over Na2SO4. Removal of the solvent left (3R,4S)-benzyl 3-(tert-butoxycarbonyl(methyl)amino)-4-tert-butyldimethylsilyloxy)pyrrolidine-1-carboxylate (123 mg, 85%) as an oil.

### Step 3. tert-butyl (3R,4S)-4-(tert-butyldimethylsilyloxy)pyrrolidin-3-yl(methyl)carbamate

A solution of (3R,4S)-benzyl 3-(tert-butoxycarbonyl(methyl)amino)-4-(tert-butyldimethylsilyloxy)pyrrolidine-1-carboxylate (123 mg, 0.27 mmol) in EtOH (40 mL) was added to 10% Pd(OH)₂ on C and shaken under H₂ (50 psi) for 4 h. The mixture was filtered through Celite and the filtrate was concentrated to afford tert-butyl (3R,4S)-4-(tert-butyldimethylsilyloxy)pyrrolidin-3-yl(methyl)carbamate (88 mg, 100%) as a dark oil.

### Preparation 17

### tert-butyl 3-methylpyrrolidin-3-ylcarbamate

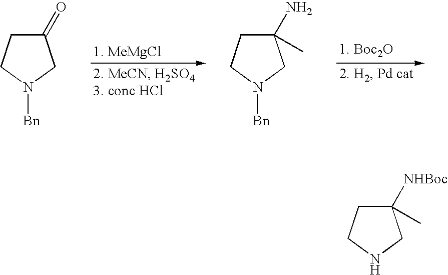

### Step 1. 1-benzyl-3-methylpyrrolidin-3-ol

A stirred solution of 1-benzylpyrrolidin-3-one (1.00 g, 5.7 mmol) in dry THF (20 mL) was cooled to −70° C. and 3 M MeMgCl in ether (4 mL, 12 mmol) was added dropwise over 2 min. The cooling bath was allowed to expire and the mixture was stirred overnight at rt. The mixture was poured into satd aq NH₄Cl (75 mL) and water (25 mL) and extracted with ether (2×100 mL). The combined ether extracts were washed with brine (25 mL) and dried over MgSO₄. Removal of the solvent left 1-benzyl-3-methylpyrrolidin-3-ol (0.90 g, 82%) as an oil.

### Step 2. N-(1-benzyl-3-methylpyrrolidin-3-yl)acetamide

1-Benzyl-3-methylpyrrolidin-3-ol (0.90 g, 4.7 mmol) was dissolved in MeCN (50 mL), cooled to −5° C. and conc. H₂SO₄ (6 mL) was added dropwise. The ice bath was allowed to melt and the mixture was stirred at rt for 3 d. The mixture was poured onto crushed ice (˜50 mL) and stirred for 0.5 h until the ice had melted. Acetonitrile was removed from the mixture on a rotary evaporator and solid K₂CO₃ was added portionwise until the mixture was basic. The mixture was extracted with CH₂Cl₂ (3×70 mL). The combined organic layers were washed with brine (30 mL), dried over Na₂SO₄ and concentrated to afford crude N-(1-benzyl-3-methylpyrrolidin-3-yl)acetamide (0.69 g, 63%) as an oil.

### Step 3. 1-benzyl-3-methylpyrrolidin-3-amine

A solution of N-(1-benzyl-3-methylpyrrolidin-3-yl)acetamide (0.69 g, 2.97 mmol) in conc. HCl (5 mL) was heated at reflux for 2 d. The dark mixture was evaporated to dryness to afford the HCl salt of 1-benzyl-3-methylpyrrolidin-3-amine as a dark solid.

### Step 4. tert-butyl 1-benzyl-3-methylpyrrolidin-3-ylcarbamate

The HCl salt of 1-benzyl-3-methylpyrrolidin-3-amine isolated in Step 3 was stirred with 10% aq K2CO3 (5 mL) and dioxane (5 mL) and Boc2O (1.23 g, 5.65 mmol) was added. The mixture was stirred for 3 d and concentrated under reduced pressure. The residue was taken up in EtOAc (90 mL), washed with water (2×20 mL) and brine (20 mL) and dried over MgSO4. Removal of the solvent left a dark brown oil (0.48 g) which was purified by chromatography on a 12-g silica cartridge eluted with a gradient from 0 to 100% EtOAc in hexanes to afford tert-butyl 1-benzyl-3-methylpyrrolidin-3-ylcarbamate (0.25 g, 22% for 2 steps) as an oil.

### Preparation 18

### tert-butyl 3-((tert-butyldimethylsilyloxy)methyl)pyrrolidin-3-ylcarbamate

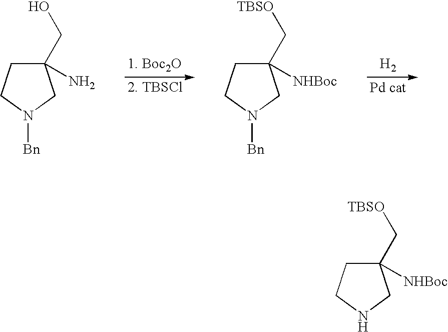

### Step 1. tert-butyl 1-benzyl-3-(hydroxymethyl)pyrrolidin-3-ylcarbamate

To a stirred solution of (3-amino-1-benzylpyrrolidin-3-yl)methanol (0.55 g, 2.7 mmol) in CH₂Cl₂ (20 mL) was added solid Boc2O (0.64 g, 2.9 mmol). The mixture was stirred overnight at rt and concentrated to afford a viscous oil which was purified by chromatography on a 12-g silica cartridge eluted with a 0-100% EtOAc in hexanes gradient to afford tert-butyl 1-benzyl-3-(hydroxymethyl)pyrrolidin-3-ylcarbamate (0.45 g, 55%) as a syrup.

### Step 2. tert-butyl 1-benzyl-3-((tert-butyldimethylsilyloxy)methyl)pyrrolidin-3-ylcarbamate

To a stirred solution of tert-butyl 1-benzyl-3-(hydroxymethyl)pyrrolidin-3-ylcarbamate (0.45 g, 1-47 mmol) and imidazole (0.21 g, 3.1 mmol) in dry DMF (5 mL) was added t-BuMe₂SiCl (0.23 g, 1.54 mmol). The mixture was stirred at rt for 18 h, diluted with ether (150 mL), washed with water (3×40 mL) and dried over Na2SO4. Removal of the solvent left an oil (0.64 g).

### Step 3. tert-butyl 3-((tert-butyldimethylsilyloxy)methyl)pyrrolidin-3-ylcarbamate

A solution of tert-butyl 1-benzyl-3-((tert-butyldimethylsilyloxy)methyl)pyrrolidin-3-ylcarbamate (0.32 g, 0.76 mmol) in methanol (50 mL) was added to 10% Pd(OH)₂ on C and shaken under 50 psi of H₂ for 2 h. The mixture was filtered through Celite and the filtrate was concentrated to afford tert-butyl 3-((tert-butyldimethylsilyloxy)methyl)pyrrolidin-3-ylcarbamate (0.23 g, 91%) as an oil

### Preparation 19

### (±)-(1R,2R)-2-(tert-butoxycarbonyl(methyl)amino)methyl)cyclopropanecarboxylic acid

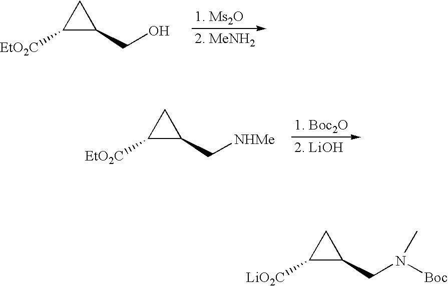

### Step 1. (±)-(1R,2R)-ethyl 2-((methylsulfonyloxy)methyl)cyclopropanecarboxylate

To a stirred solution of (±)-(1R,2R)-ethyl 2-hydroxymethyl)cyclopropanecarboxylate (130 mg, 0.90 mmol, prepared as described in WO 02/066446 Example 4) and pyridine (0.17 mL, 2.0 mmol) in CH₂Cl₂ (10 mL) cooled in an ice bath was added solid methanesulfonic anhydride (173 mg, 0.99 mmol). The cooling bath was allowed to melt and the mixture was stirred overnight at rt. The mixture was diluted with ether (90 mL), washed with 5% aq HCl (20 mL) and satd aq NaHCO₃ (20 mL) and dried over MgSO₄. Removal of the solvent left (±)-(1R,2R)-ethyl 2-((methylsulfonyloxy)methyl)cyclopropanecarboxylate (165 mg, 83%) as an oil.

### Step 2. (±)-(1R,2R)-ethyl 2-((methylamino)methyl)cyclopropanecarboxylate

To a solution of (±)-(1R,2R)-ethyl 2-((methylsulfonyloxy)methyl)cyclopropanecarboxylate (165 mg, 0.74 mmol) in MeCN (0.5 mL) was added 30 wt % MeNH₂ in EtOH (1.5 mL). The mixture was heated at 100° C. in a microwave for 10 min and concentrated to leave crude (1R,2R)-ethyl 2-((methylamino)methyl)cyclopropanecarboxylate as an oil.

### Step 3. (±)-(1R,2R)-ethyl 2-((tert-butoxycarbonyl(methyl)amino)methyl)cyclopropanecarboxylate

Crude (1R,2R)-ethyl 2-(methylamino)methyl)cyclopropanecarboxylate from Step 2 was dissolved in dioxane (3 mL) and 10% aq K₂CO₃ (3 mL) and Boc₂O (250 mg, 1.15 mmol) was added. The mixture was stirred overnight at rt, diluted with brine (20 mL) and extracted with ether (90 mL). The ether layer was dried over MgSO₄ and concentrated to afford leave an oil (234 mg) which was purified on a 12-g silica cartridge eluted with a gradient from 0 to 80% EtOAc in hexanes to afford (1R,2R)-ethyl 2-((tert-butoxycarbonyl(methyl)amino)-methyl)cyclopropanecarboxylate (86 mg, 45% for 2 steps) as an oil.

### Step 4. (±)-(1R,2R)-2-((tert-butoxycarbony(methyl)amino)methyl)cyclopropanecarboxylic acid

To a solution of (1R,2R)-ethyl 2-(tert-butoxycarbonyl(methyl)amino)methyl)cyclopropanecarboxylate (86 mg, 0.33 mmol) in THF (2 mL) and EtOH (4 mL) was added a solution of LiOH.H2O (14 mg, 0.33 mmol) in water (2 mL). The mixture was stirred at rt overnight and evaporated to dryness to leave the lithium salt of (±)-(1R,2R)-2-((tert-butoxycarbonyl(methyl)amino)methyl)cyclopropanecarboxylic acid (79 mg, quant) as a tacky solid.

The following intermediates were prepared using procedures analogous to those described above:

(±)-(1R,2R,3R)-2-((tert-butoxycarbonyl(methyl)amino)methyl)-3-methylcyclopropanecarboxylic acid using (±)-(1R,2R,3R)-methyl 2-(hydroxymethyl)-3-methylcyclopropanecarboxylate in Step 1.

(±)-(1R,2R)-2-((tert-butoxycarbonyl(methyl)amino)methyl)-1-methylcyclopropanecarboxylic acid using (±)-1R,2R)-methyl 2-(hydroxymethyl)-1-methylcyclopropanecarboxylate in Step 1.

(±)-(1R,2R)-2-(tert-butoxycarbonyl(methyl)amino)methyl)-2-methylcyclopropanecarboxylic acid using (±)-(1R,2R)-methyl 2-(hydroxymethyl)-2-methylcyclopropanecarboxylate in Step 1.

### Preparation 20

### (2S)-2-(1-(6-fluoro-3′-methylbiphenyl-2-yl)-5-methoxypentyl)morpholine

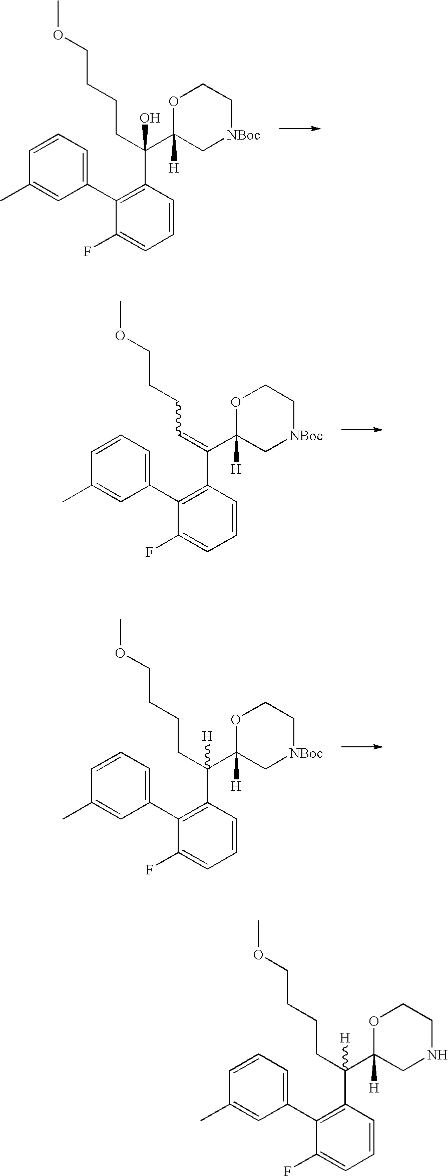

### Step 1. (S)-tert-butyl 2-(1-(6-fluoro-3′-methylbiphenyl-2-yl)-5-methoxypent-1-enyl)morpholine-4-carboxylate

A mixture of (R)-tert-butyl 2-((R)-1-(6-fluoro-3′-methylbiphenyl-2-yl)-1-hydroxy-5-methoxypentyl)morpholine-4-carboxylate (188 mg, 0.39 mmol) and Burgess' reagent (186 mg, 0.78 mmol) in toluene (3 mL) was heated to reflux under a N₂ atmosphere for 2 h, then cooled to rt and diluted with EtOAc, washed with H₂O and brine, dried over Na₂SO₄, filtered and evaporated. The residue was purified by flash chromatography to give (S)-tert-butyl 2-(1-(6-fluoro-3′-methylbiphenyl-2-yl)-5-methoxypent-1-enyl)morpholine-4-carboxylate (133 mg, 73%). MS m/z 470 (M+H)⁺.

### Step 2. (2S)-tert-butyl 2-(1-(6-fluoro-3′-methylbiphenyl-2-yl)-5-methoxypentyl)morpholine-4-carboxylate

(S)-tert-butyl 2-(1-(6-fluoro-3′-methylbiphenyl-2-yl)-5-methoxypent-1-enyl)morpholine-4-carboxylate (133 mg, 0.28 mmol) was dissolved in methanol and hydrogenated under 50 psi of hydrogen in the presence of 10% Pd(OH)₂/C as catalyst for 48 h. The reaction mixture was filtered and evaporated to give (2S)-tert-butyl 2-(1-(6-fluoro-3′-methylbiphenyl-2-yl)-5-methoxypentyl)morpholine-4-carboxylate in nearly quantitative yield. MS m/z 470 (M+H)⁺.

### Step 3. (2S)-2-(1-(6-fluoro-3′-methylbiphenyl-2-yl)-5-methoxypentyl)morpholine

(2S)-tert-butyl 2-(1-(6-fluoro-3′-methylbiphenyl-2-yl)-5-methoxypentyl)morpholine-4-carboxylate from Step 2 was dissolved in 1 M HCl in MeOH and stirred at 50° C. for 10 min, the solvent was removed under reduced pressure to give (2S)-2-(1-(6-fluoro-3′-methylbiphenyl-2-yl)-5-methoxypentyl)morpholine as its HCl salt in quantitative yield. MS m/z 494 (M+Na)⁺.

### Preparation 21

### tert-butyl (3R,4R)-4-(tert-butyldimethylsilyloxy)pyrrolidin-3-ylcarbamate

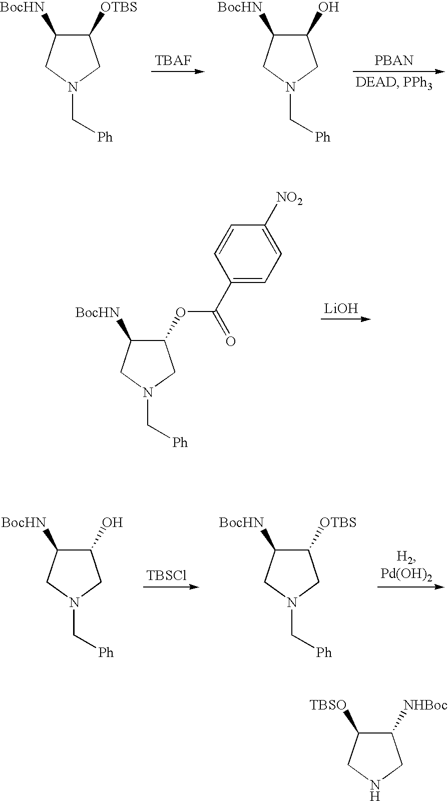

### Step 1. tert-butyl (3R,4S)-1-benzyl-4-hydroxypyrrolidin-3-ylcarbamate

To a solution of tert-butyl (3R,4S)-1-benzyl-4-tert-butyldimethylsilyloxy)pyrrolidin-3-ylcarbamate (1.50 g, 3.69 mmol) in acetonitrile (20 mL) was added TBAF (1.45 g, 5.54 mmol) in one portion. The reaction mixture was warmed to 60° C. and was stirred at this temperature for 3 h. The solvents were removed in vacuo to leave a residue, which was purified by chromatography to afford pure tert-butyl (3R,4S)-1-benzyl-4-hydroxypyrrolidin-3-ylcarbamate (1.05 g, 97%).

### Step 2. (3R,4R)-1-benzyl-4-(tert-butoxycarbonylamino)pyrrolidin-3-yl 4-nitrobenzoate

A 100-mL, three-necked, round-bottomed flask was equipped with a stirring bar, nitrogen inlet, rubber septum, and thermometer. The flask was charged with tert-butyl (3R,4S)-1-benzyl-4-hydroxypyrrolidin-3-ylcarbamate (1.00 g, 3.42 mmol), 4-nitrobenzoic acid (572 mg, 3.42 mmol), triphenylphosphine (1.08 g, 4.12 mmol), and THF (20 mL). The flask was immersed in an ice bath and diethyl azodicarboxylate (715 mg, 4.12 mmol) was added dropwise at a rate such that the temperature of the reaction mixture was maintained below 10° C. Upon completion of the addition, the flask was removed from the ice bath and the solution was allowed to stir at rt overnight (14 h). The reaction mixture was diluted with ether (20 mL), and washed with satd aq NaHCO₃ (2×40 mL). The aqueous layers were combined and back-extracted with ether (40 mL). The combined organic layers were dried over Na₂SO₄. Excess solvent and other volatile reaction components were completely removed under reduced pressure initially on a rotary evaporator and then under high vacuum (approximately 0.2 mm for 3 hr at 30° C.). The resulting semi-solid was suspended in ether (15 mL) and allowed to stand at rt overnight. The mixture was stirred while hexane (8 mL) was slowly added. The resulting white solid was filtered under vacuum and the filter cake was washed with 50% (v/v) ether-hexanes (66 mL). The solvent was removed from the filtrate on a rotary evaporator under reduced pressure to give a yellow oil that was dissolved in methylene chloride (10 mL) and diluted with 8% ether-hexanes (15 mL). The solution was applied to a flash chromatography column and eluted with 8% ether-hexanes to give pure (3R,4R)-1-benzyl-4-(tert-butoxycarbonylamino)pyrrolidin-3-yl 4-nitrobenzoate as a white crystalline solid (1.10 g, 73%). ¹H NMR (400 MHz, MeOD): 1.416 (s, 9H), 2.30-2.40 (t, 1H), 2.78-2.86 (m, 1H), 2.88-3.00 (m, 1H), 3.10-3.20 (t, 1H), 3.60-3.70 (m, 2H), 4.18-4.30 (m, 1H), 5.19-5.30 (s, 1H), 7.20-7.38 (m, 5H), 8.20-8.40 (m, 4H). MS (E/Z): 442 (M+H⁺)

### Step 3. tert-butyl (3R,4R)-1-benzyl-4-hydroxypyrrolidin-3-ylcarbamate

To a solution of (3R,4R)-1-benzyl-4-tert-butoxycarbonylamino)pyrrolidin-3-yl 4-nitrobenzoate (1.05 g, 2.38 mmol) in ethanol (40 mL), water (20 mL) and THF (40 mL) was added LiOH.H₂O (100 mg, 2.38 mmol). The mixture was stirred for 1 h at rt. The mixture was diluted with ether (100 mL), quenched with satd aq NH₄Cl (100 mL), extracted with EtOAc (3×150 mL). The combined organic layers were dried over Na₂SO₄, filtered and concentrated to give crude tert-butyl (3R,4R)-1-benzyl-4-hydroxypyrrolidin-3-ylcarbamate (610 mg, 88%), which was used in the next step without further purification.

### Step 4. tert-butyl (3R,4R)-1-benzyl-4-(tert-butyldimethylsilyloxy)pyrrolidin-3-ylcarbamate

To a stirred solution of tert-butyl (3R,4R)-1-benzyl-4-hydroxypyrrolidin-3-ylcarbamate (600 mg, 2.05 mmol) and imidazole (280 mg, 4.10 mmol) in DMF (10 mL) was added tert-butyl-chloro-dimethyl-silane (367 mg, 2.45 mmol). The mixture was stirred overnight at rt, diluted with ether (10 mL) and washed with water (40 mL). The aqueous layer was extracted with ether (20 mL). The combined organic layers were dried over Na₂SO₄ and concentrated to give the crude product, which was purified by column chromatography to afford pure tert-butyl (3R,4R)-1-benzyl-4-tert-butyldimethylsilyloxy)pyrrolidin-3-ylcarbamate (630 mg, 76%). ¹H NMR (400 MHz, CDCl₃): 2.12-2.21 (m, 1H), 2.50-2.80 (m, 2H), 3.10-3.20 (m, 1H), 3.61 (s, 1H), 3.70-3.905 (m, 1H), 4.00-4.09 (s, 1H), 4.60-5.00 (m, 1H); MS (E/Z): 407 (M+H⁺).

### Step 5. tert-butyl (3R,4R)-4-(tert-butyldimethylsilyloxy)pyrrolidin-3-ylcarbamate

A solution of tert-butyl (3R,4R)-1-benzyl-4-(tert-butyldimethylsilyloxy)pyrrolidin-3-ylcarbamate (600 mg, 1.48 mmol) in methanol (15 mL) was added to 20% Pd(OH)₂/C (300 mg). The mixture was hydrogenated under 50 psi for 3 h and filtered through celite. The filtrate was evaporated to give tert-butyl (3R,4R)-4-(tert-butyldimethylsilyloxy)pyrrolidin-3-ylcarbamate (410 mg, yield 88%). ¹H NMR (400 MHz, CDCl₃): 2.250-2.350 (m, 1H), 2.6-2.7 (m, 1H), 2.7-2.8 (m, 1H), 3.11-3.21 (m, 1H), 3.80-3.90 (m, 1H), 4.00-4.08 (s, 1H), 4.80-5.35 (m, 1H), MS (E/Z): 317 (M+H⁺)

### Preparation 22

### (±)-(1R,2R)-2-((tert-butoxycarbonylamino)methyl)cyclopropanecarboxylic acid

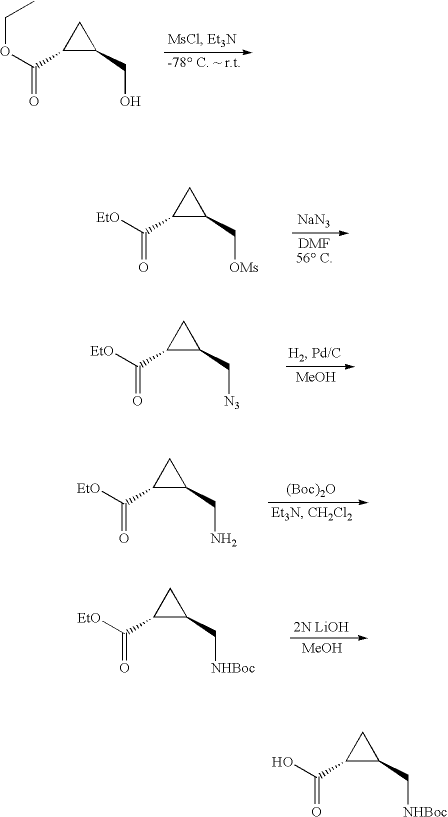

### Step 1. (±)-(1R,2R)-ethyl 2-((methylsulfonyloxy)methyl)cyclopropanecarboxylate

A solution of (1R,2R)-ethyl 2-(hydroxymethyl)cyclopropanecarboxylate (933 mg, 6.479 mmol) in CH₂Cl₂ (80 mL) was cooled to −78° C. and triethylamine (1.81 mL, 2 equiv) was added. Methanesulfonyl chloride (530 μL, 1.05 equiv) was added dropwise. After 20 min, the reaction mixture was allowed to warm slowly to rt. After 2 h, the mixture was diluted with CH₂Cl₂ (200 mL), washed with 5% aq HCl (2×30 mL), satd aq NaHCO₃ (25 mL) and brine (20 mL), and dried over Na₂SO₄. Concentration afforded (1R,2R)-ethyl 2-((methylsulfonyloxy)methyl)cyclopropanecarboxylate which was used without purification. LC/MS (3 min) t_{R}=1.21, m/z 223 (M+1).

### Step 2. (±)-(1R,2R)-ethyl 2-(azidomethyl)cyclopropanecarboxylate

(1R,2R)-ethyl 2-((methylsulfonyloxy)methyl)cyclopropanecarboxylate from Step 1 sodium azide (850 mg, 2 equiv) were mixed with dry DMF (25 mL) and heated overnight at 56° C. LC/MS showed complete reaction had occurred. The mixture was diluted with ether (200 mL), washed with water (50 mL) and brine (20 mL), and dried over Na₂SO₄. After concentration, the residue was purified by chromatography on silica gel (40 g column, 0 to 25% EtOAc in Hexanes gradient) to afford (±)-(1R,2R)-ethyl 2-(azidomethyl)cyclopropanecarboxylate (0.77 g, 70% for two steps). ¹H NMR (CDCl₃) δ 4.11 (q, 2H), 3.20 (t, 2H), 1.69 (m, 1H), 1.56 (m, 1H), 1.24 (m, 4H), 0.87 (m, 1H).

### Step 3. (±)-(1R,2R)-ethyl 2-(tert-butoxycarbonylamino)methyl)cyclopropanecarboxylate

(±)-(1R,2R)-ethyl 2-(azidomethyl)cyclopropanecarboxylate (0.77 g, 4.56 mmol), 10% Pd/C (ca 30 mg) and methanol (40 mL) were mixed and shaken under 25 psi of hydrogen for 30 min. The mixture was filtered and the filtrate was evaporated to leave (±)-(1R,2R)-ethyl 2-(aminomethyl)cyclopropanecarboxylate (0.51 g, 78%). This material was dissolved in CH₂Cl₂ (30 mL) and (Boc)₂O (856 mg, 1.1 equiv) and triethylamine (500 μL, 1.0 equiv) were added. The mixture was stirred overnight at rt. The mixture was concentrated and purified by chromatography on silica gel (40 g column, 0 to 35% EtOAc in Hexanes gradient) to afford product (±)-(1R,2R)-ethyl 2-(tert-butoxycarbonylamino)methyl)cyclopropanecarboxylate (822 mg, 95%). LC-MS (3 min) t_{R}=1.55 min., t/z 266 (M+Na).

### Step 4. (±)-(1R,2R)-2-((tert-butoxycarbonylamino)methyl)cyclopropanecarboxylic acid

To a solution of (±)-(1R,2R)-ethyl 2-(tert-butoxycarbonylamino)methyl)cyclopropanecarboxylate (440 mg, 1.81 mmol) in methanol (4 mL) was added 2 N aq LiOH (1.81 mL, 2 equiv) solution. The mixture was stirred overnight at rt. The mixture was concentrated and the residue was partitioned between CH₂Cl₂ (50 mL) and water (20 mL). The aqueous layer was acidified with 5% aq HCl and extracted with CH₂Cl₂ (3×10 mL). The combined organic layers were concentrated and used for next step without purification. LC-MS (3 min) t_{R}=1.25 min, m/z 216 (M+1).

### Preparation 23

### (S)-1-(3-fluoro-2-piperidin-1-yl)phenyl)-5-methoxy-1-((R)-piperidin-3-yl)pentan-1-ol

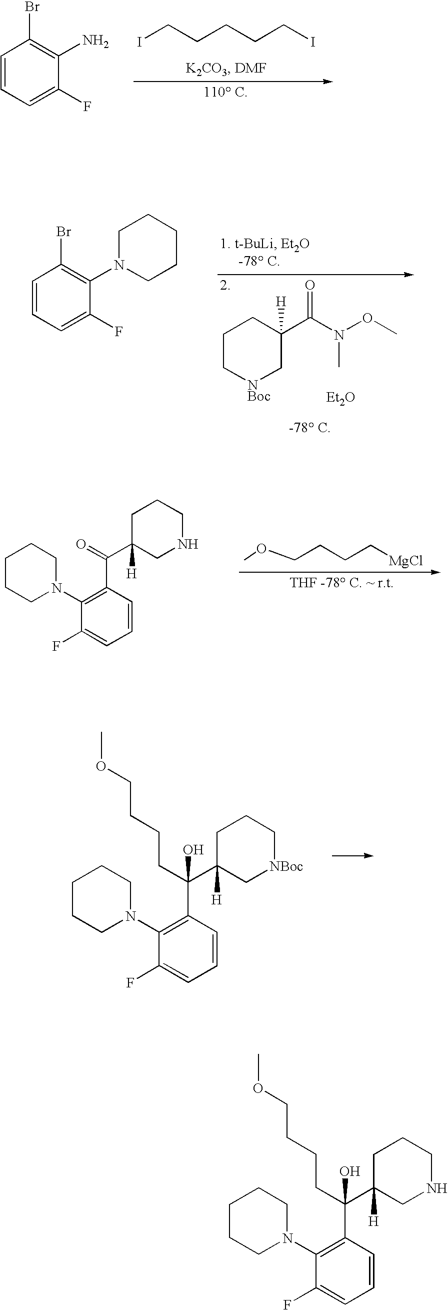

### Step 1. 1-(2-bromo-6-fluorophenyl)piperidine

2-Bromo-6-fluoroaniline (3.0 mL, 26.4 mmol), 1,5-diiodopentane (3.93 mL, 1.0 equiv), K₂CO₃ (7.3 g, 2.0 equiv) were mixed with anhydrous DMF (80 mL) and heated overnight at 110° C. LC-MS indicated that product had formed. The mixture was cooled to rt, diluted with ether (200 mL) and washed by water (100 mL). The water layer was extracted with 1:1 Ether/EtOAc (2×50 mL). The combined organic layers were washed with water (100 mL) water and brine (50 mL), and dried over Na₂SO₄. After concentration, the residue was purified by flash chromatography (120 g silica gel column, 0 to 20% EtOAc in Hexanes gradient) to afford 1-(2-bromo-6-fluorophenyl)piperidine (1.11 g, 16%). LC-MS (3 min) t_{R}=2.52 min. ¹H NMR (CDCl₃) δ 7.35 (d, 1H), 6.98 (m, 1H), 6.89 (m, 1H), 3.18 (s, 4H), 1.84-1.47 (m, 6H). ¹³C NMR (CDCl₃) δ 162.3, 159.8, 139.3, 139.1, 128.9, 125.5, 125.1, 116.4, 116.2, 52.5, 26.9, 24.5.

### Step 2. (R)-tert-butyl 3-(3-fluoro-2-(piperidin-1-yl)benzoyl)piperidine-1-carboxylate

Under protection of N₂ gas, a solution of 1-(2-bromo-6-fluorophenyl)piperidine (110 mg, 0.43 mmol) in anhydrous ether (4 mL) was cooled to −78° C. and 1.7 M t-BuLi in pentane (556 μL, 2.2 equiv) was added slowly over 5 min. After 10 min, LC-MS showed the starting material peak had disappeared while a more polar peak had appeared. A solution of (R)-tert-butyl 3-(methoxy(methyl)carbamoyl)piperidine-1-carboxylate (117 mg, 1 equiv) in anhydrous ether (3 mL) was added slowly. After 30 min, the reaction mixture was warmed up to rt slowly. The mixture was stirred for 1 h at rt and quenched with satd aq NH₄Cl. The organic layer was diluted with ether (50 mL) and the layers were separated. The aqueous layer was extracted with ether (2×10 mL). The combined ether layers were washed with brine (20 mL) and dried over Na₂SO₄. After concentration, the residue was purified by flash chromatography (12 g silica gel column, 0 to 25% EtOAc in Hexanes gradient). The second UV active peak eluted was collected and concentrated to afford (R)-tert-butyl 3-(3-fluoro-2-(piperidin-1-yl)benzoyl)piperidine-1-carboxylate (73 mg, 44%). LC-MS (3 min) t_{R}=2.35 min, m/z 291 (M+1).

### Step 3. (R)-tert-butyl 3-((S)-1-(3-fluoro-2-piperidin-1-yl)phenyl)-1-hydroxy-5-methoxypentyl)piperidine-1-carboxylate

Under protection of N₂ gas, a solution of (R)-tert-butyl 3-(3-fluoro-2-(piperidin-1-yl)benzoyl)piperidine-1-carboxylate (73 mg, 0.187 mmol) in dry THF (5 mL) was cooled to −78° C. and 1.47 M 4-methoxybutylmagnesium chloride in THF (255 μL, 2.0 equiv) was added slowly. After 10 min, the reaction mixture was warmed up rt slowly. The mixture was stirred for 2 h at rt and quenched with satd aq NH₄Cl. The mixture was diluted with ether (50 mL), washed with brine (20 mL), and dried over Na₂SO₄. After concentration, the residue was purified by preparative HPLC to afford (R)-tert-butyl 3-((S)-1-(3-fluoro-2-piperidin-1-yl)phenyl)-1-hydroxy-5-methoxypentyl)piperidine-1-carboxylate (53.3 mg, 60%). LC-MS (3 min) t_{R}=1.93 min, m/z 479 (M+1).

### Step 4. (S)-1-(3-fluoro-2-(piperidin-1-yl)phenyl)-5-methoxy-1-((R)-piperidin-3-yl)pentan-1-ol

(R)-tert-butyl 3-((S)-1-(3-fluoro-2-(piperidin-1-yl)phenyl)-1-hydroxy-5-methoxypentyl)piperidine-1-carboxylate (53.3 mg, 0.11 mmol) was dissolved in 1:1 mixture of acetonitrile and 2 N aq HCl. The reaction mixture was stirred overnight at rt. LC-MS showed the reaction was complete. 5% aq NaOH solution was added to basify the mixture to pH=˜10. The acetonitrile was removed under vacuum. The aqueous residue was extracted with CH₂Cl₂ (3×15 mL). The combined organic layers were dried over Na₂SO₄. After concentration, the crude product was used without purification.

### Preparation 24

### Methyl (4S)-4-(6-chloro-3′-methylbiphenyl-2-yl)-4-hydroxy-4-(piperidin-3-yl)butylcarbamate

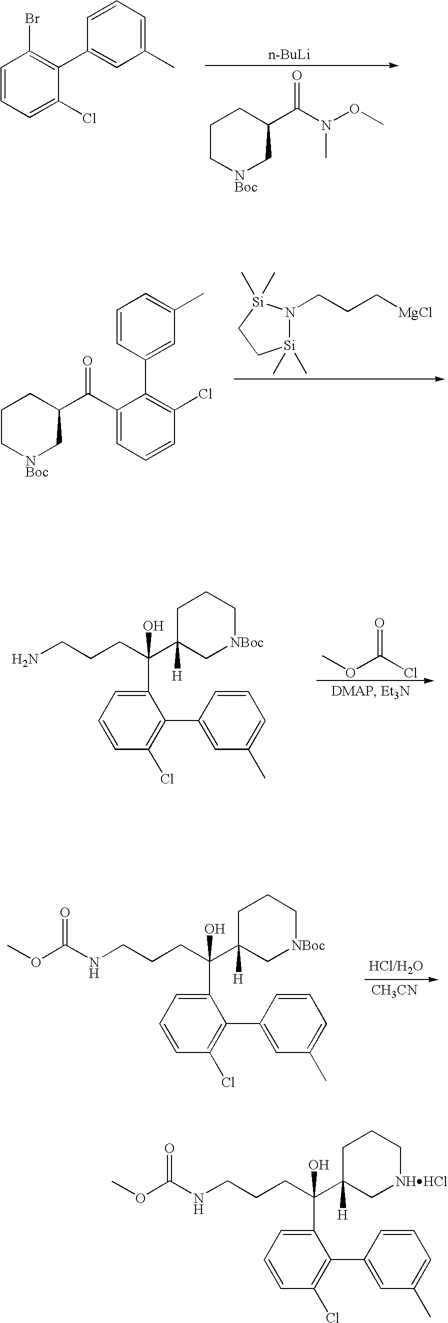

### Step 1. (R)-tert-butyl 3-(6-chloro-3′-methylbiphenylcarbonyl)piperidine-1-carboxylate

To a solution of 6-bromo-2-fluoro-3′-methylbiphenyl (2 g, 7.14 mmol) in anhydrous THF (30 mL) cooled to −78° C. was added dropwise a solution of 1.6 M of n-BuLi in hexane (4.46 mL). The reaction mixture was stirred at −78° C. for 1 h and a solution of (R)-tert-butyl 3-(methoxy(methyl)carbamoyl)piperidine-1-carboxylate (1.94 g, 7.14 mmol) in anhydrous THF (20 mL) was added. The mixture was allowed to warm to rt and stirred overnight. The mixture was quenched with satd aq NH₄Cl (40 mL) and extracted with EtOAc (40 mL). The combined organic layers were dried over Na₂SO₄ and concentrated to give crude product, which was purified by flash column chromatography to afford (R)-tert-butyl 3-(6-chloro-3′-methylbiphenylcarbonyl)piperidine-1-carboxylate (1 g, 34%). ¹H NMR (400 MHz, CD₃OD): 0.80-1.20 (m, 8H), 1.30 (s, 1H), 1.40 (s, 1H), 1.40-1.60 (m, 2H), 2.00-2.18 (s, 1H), 2.30-2.40 (s, 3H), 2.60-2.80 (m, 2H), 3.50-3.80 (m, 2H), 7.00-7.15 (s, 2H), 7.20-7.30 (d, 1H), 7.30-7.40 (t, 2H), 7.39-7.48 (t, 1H), 7.60-7.70 (d, 1H); MS (E/Z): 414 (M+H⁺)

### Step 2. (R)-tert-butyl 3-((S)-4-amino-1-(6-chloro-3′-methylbiphenyl-2-yl)-1-hydroxybutyl)piperidine-1-carboxylate

To a solution of (R)-tert-butyl 3-(6-chloro-3′-methylbiphenylcarbonyl)piperidine-1-carboxylate (800 mg, 1.94 mmol) in anhydrous THF (15 mL) cooled to −78° C. was added dropwise a solution of 2 M (3-(2,2,5,5-tetramethyl-1,2,5-azadisilolidin-1-yl)propyl)magnesium chloride in THF (0.968 mL, 1.94 mmol). After addition, the reaction mixture was allowed to warm slowly to rt while stirring overnight. The mixture was quenched with satd aq NH₄Cl (15 mL) and extracted with CH₂Cl₂ (3×). The combined organic layers were dried over Na₂SO₄ and concentrated to give crude (R)-tert-butyl 3-((S)-4-amino-1-(6-chloro-3′-methylbiphenyl-2-yl)-1-hydroxybutyl)piperidine-1-carboxylate (900 mg), which was used in the next step without further purification.

### Step 3. (R)-tert-butyl 3-((S)-1-(6-chloro-3′-methylbiphenyl-2-yl)-1-hydroxy-4-(methoxycarbonylamino)butyl)piperidine-1-carboxylate

To a solution of (R)-tert-butyl 3-((S)-4-amino-1-(6-chloro-3′-methylbiphenyl-2-yl)-1-hydroxybutyl)piperidine-1-carboxylate (800 mg, 1.69 mmol) in anhydrous CH₂Cl₂ (15 mL) were added 4-dimethylaminopyridine (1.24 g, 10.17 mmol) and Et₃N (2.35 mL, 16.95 mmol). The mixture was cooled with an ice bath and methyl chloroformate (0.65 mL, 8.47 mmol) in CH₂Cl₂ (5 mL) was added. The reaction mixture was allowed to warm slowly to rt while stirring overnight. The solvent was removed in vacuo and the residue was purified by column chromatography to afford (R)-tert-butyl 3-((S)-1-(6-chloro-3′-methylbiphenyl-2-yl)-1-hydroxy-4-(methoxycarbonylamino)butyl)piperidine-1-carboxylate (700 mg, 78%). ¹H NMR (400 MHz, CD₃OD): 1.00-1.70 (m, 17H), 2.30-2.50 (d, 3H), 2.50-2.70 (s, 1H), 2.90-2.31 (m, 2H), 3.50-3.52 (m, 3H), 3.80-4.20 (m, 2H), 6.0-7.15 (m, 3H), 7.15-7.40 (m, 3H), 7.50-7.70 (m, 1H); MS (E/Z): 531 (M+H⁺)

### Step 4. Methyl (4S)-4-(6-chloro-3′-methylbiphenyl-2-yl)-4-hydroxy-4-piperidin-3-yl)butylcarbamate

To a solution of (R)-tert-butyl 3-((S)-1-(6-chloro-3′-methylbiphenyl-2-yl)-1-hydroxy-4-(methoxycarbonylamino)butyl)piperidine-1-carboxylate (600 mg, 1.13 mg) in CH₃CN (18 mL) was added 2N aq HCl (15 mL) and the reaction mixture was vigorously stirred overnight at rt. The solvents were removed in vacuo to give methyl (4S)-4-(6-chloro-3′-methylbiphenyl-2-yl)-4-hydroxy-4-piperidin-3-yl)butylcarbamate as its hydrochloride salt (500 mg, 95.8%). ¹H NMR (400 MHz, CD₃OD): 1.00-1.20 (m, 1H), 1.30-1.80 (m, 8H), 1.80-2.00 (m, 2H), 2.40-2.50 (d, 3H), 2.75-2.90 (t, 1H), 2.90-3.05 (m, 3H), 3.05-3.12 (t, 1H), 3.20-3.30 (m, 1H), 3.30-3.40 (m, 1H), 3.60-3.70 (d, 4H), 6.90-6.98 (d, 1H), 7.00-7.12 (m, 1H), 7.25-7.50 (m, 4H), 7.75-7.85 (d, 1H); MS (E/Z): 431 (M+H⁺)

The following piperidines were prepared using procedures analogous to those described above:

N—((S)-4-(6-fluoro-3′-methylbiphenyl-2-yl)-4-hydroxy-4-((R)-piperidin-3-yl)butyl)acetamide using acetyl chloride in place of methyl chloroformate in Step 3.

N—((S)-4-(biphenyl-2-yl)-4-hydroxy-4-((R)-piperidin-3-yl)butyl)acetamide using 2-bromobiphenyl in Step 1 and acetyl chloride in place of methyl chloroformate in Step 3.

N—((S)-4-(3′-chloro-6-methylbiphenyl-2-yl)-4-hydroxy-4-((R)-piperidin-3-yl)butyl)acetamide using 6-bromo-2-chloro-3′-methylbiphenyl in Step 1 and acetyl chloride in place of methyl chloroformate in Step 3.

Methyl (S)-4-(6-chloro-3′-methylbiphenyl-2-yl)-4-hydroxy-4-((R)-piperidin-3-yl)butylcarbamate using 6-bromo-2-chloro-3′-methylbiphenyl in Step 1.

N-((4S)-4-(2′,6-difluoro-5′-methylbiphenyl-2-yl)-4-hydroxy-4-((R)-piperidin-3-yl)butyl)acetamide using 2′-bromo-2,6′-difluoro-5-methylbiphenyl in Step 1 and acetyl chloride in place of methyl chloroformate in Step 3.

Methyl (S)-4-hydroxy-4-((R)-piperidin-3-yl)-4-(2-(pyridin-3-yl)phenyl)butylcarbamate using 3-(2-bromophenyl)pyridine in Step 1.

Methyl (S)-4-hydroxy-4-((R)-piperidin-3-yl)-4-(2-(pyridin-4-yl)phenyl)butylcarbamate using 4-(2-bromophenyl)pyridine in Step 1.

N—((S)-hydroxy-4-((R)-piperidin-3-yl)-4-(2-(o-tolyloxy)phenyl)butyl)acetamide using 1-bromo-2-(o-tolyloxy)benzene in Step 1 and acetyl chloride in place of methyl chloroformate in Step 3.

Methyl (S)-4-hydroxy-4-((R)-piperidin-3-yl)-4-(2-(o-tolyloxy)phenyl)butylcarbamate using 1-bromo-2-(o-tolyloxy)benzene in Step 1.

Methyl (S)-4-(3′-ethyl-6-fluorobiphenyl-2-yl)-hydroxy-4-((R)-piperidin-3-yl)butylcarbamate using 2-bromo-3′-ethyl 6-fluorobiphenyl in Step 1.

Methyl (S)-4-(6-fluoro-3′-methoxybiphenyl-2-yl)-4-hydroxy-4-((R)-piperidin-3-yl)butylcarbamate using 2-bromo-6-fluoro-3′-methoxybiphenyl in Step 1.

Methyl (S)-4-(6-chloro-3′-isopropylbiphenyl-2-yl)-4-hydroxy-4-((R)-piperidin-3-yl)butylcarbamate using 2-bromo-4-chloro-3′-isopropylbiphenyl in Step 1.

Methyl (S)-4-(6-chloro-3′-methoxybiphenyl-2-yl)-4-hydroxy-4-((R)-piperidin-3-yl)butylcarbamate using 2-bromo-6-chloro-3′-methoxybiphenyl in Step 1.

Methyl (S)-4-(6-chloro-3′-methylbiphenyl-2-yl)-4-hydroxy-4-((R)-piperidin-3-yl)butylcarbamate using 2-bromo-6-chloro-3′-methylbiphenyl in Step 1.

Methyl (S)-4-(3-chloro-2-quinolin-3-yl)phenyl)-4-hydroxy-4-((R)-piperidin-3-yl)butylcarbamate using 3-(2-bromo-6-chlorophenyl)quinoline in Step 1.

Methyl (S)-4-(6-chloro-3′-ethylbiphenyl-2-yl)-4-hydroxy-4-((R)-piperidin-3-yl)butylcarbamate using 2-bromo-6-chloro-3′-ethylbiphenyl in Step 1.

N—(S)-4-(6-chloro-3′-ethylbiphenyl-2-yl)-4-hydroxy-4-((R)-piperidin-3-yl)butyl)acetamide using 2-bromo-6-chloro-3′-ethylbiphenyl in Step 1 and acetyl chloride in place of methyl chloroformate in Step 3.

Methyl (4S)-4-(2′,6-difluoro-5′-methylbiphenyl-2-yl)-4-hydroxy-4-((R)-piperidin-3-yl)butylcarbamate using 2′-bromo-2,6′-difluoro-5-methylbiphenyl in Step 1.

Methyl (S)-4-(3-chloro-2-(o-tolyloxy)phenyl)-4-hydroxy-4-((R)-piperidin-3-yl)butylcarbamate using 1-bromo-3-chloro-2-(o-tolyloxy)benzene in Step 1.

Methyl (S)-4-(3-chloro-2-(2-ethylphenoxy)phenyl)-4-hydroxy-4-((R)-piperidin-3-yl)butylcarbamate using 1-bromo-3-chloro-2-(2-ethylphenoxy)benzene in Step 1.

### Preparation 25

### (S)-1-(2-cyclohexenyl-3-fluorophenyl)-5-methoxy-1-((R)-piperidin-3-yl)pentan-1-ol

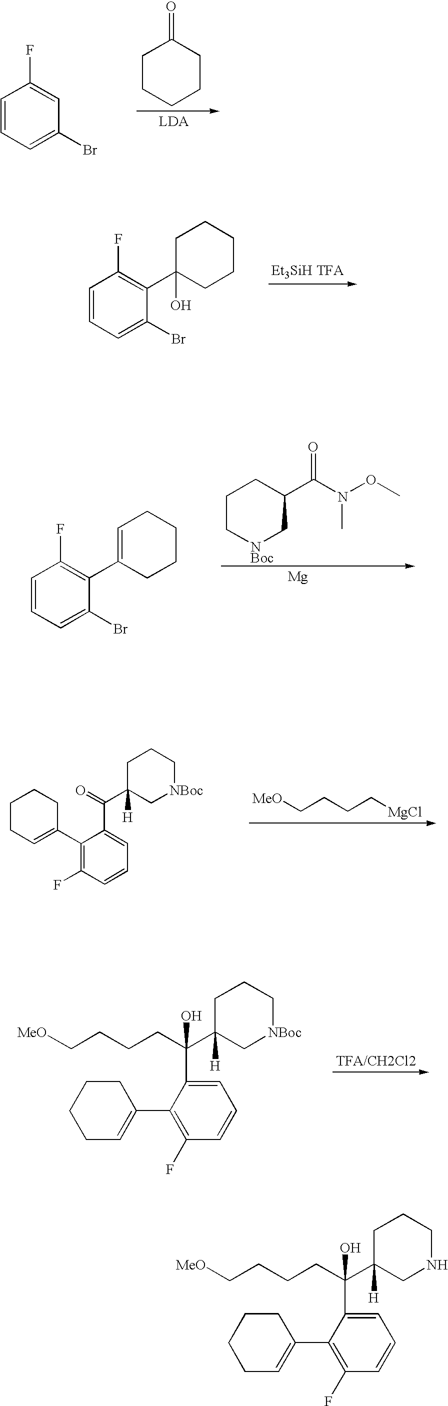

### Step 1. 1-(2-bromo-6-fluorophenyl)cyclohexanol

A solution of diisopropylamine (5.76 g, 57 mmol) in anhydrous THF (50 mL) under N₂ was cooled to −78° C. and 2.5 M n-BuLi solution in hexane (22.8 mL, 57 mmol) was added dropwise slowly. The reaction mixture was stirred at −78° C. for 1 h. A solution of 1-bromo-3-fluorobenzene (10 g, 57 mmol) in anhydrous THF (70 mL) was added dropwise slowly and the mixture was stirred at −78° C. for 2 h. A solution of cyclohexanone (4.7 g, 47 mmol) in anhydrous THF (70 mL) was added dropwise and the reaction mixture was warmed to rt and stirred overnight. The mixture was quenched with satd aq NH₄Cl (100 mL) and extracted with EtOAc (3×). The combined organic extracts were dried over Na₂SO₄, concentrated and purified by flash column chromatography to afford 1-(2-bromo-6-fluorophenyl)cyclohexanol (4.5 g, 29%). ¹H NMR (400 MHz, CDCl₃): 1.60-1.62 (m, 3H), 1.70-1.81 (m, 1H), 1.83-1.86 (m, 2H), 2.13-2.19 (m, 4H), 2.92 (m, 1H), 6.96-7.06 (m, 2H), 7.40-7.42 (m, 1H).

### Step 2. 1-bromo-2-cyclohexenyl)-3-fluorobenzene

1-(2-bromo-6-fluorophenyl)cyclohexanol (1 g, 3.7 mmol) was dissolved in anhydrous toluene (10 mL), (methoxycarbonylsulfamoyl)triethylammonium hydroxide, inner salt (Burgess Reagent, 2 g, 8.4 mmol) was added. The reaction mixture was stirred and heated under reflux for 24 h. The upper clear layer was collected, and the remainder was extracted with EtOAc (3×). The organic layers were combined and concentrated. The residue was purified by flash column chromatography to afford 1-bromo-2-cyclohexenyl)-3-fluorobenzene (0.8 g, 86%). ¹H NMR (400 MHz, CDCl₃): 1.68-1.82 (m, 4H), 2.19-2.20 (m, 4H), 5.64-5.65 (m, 1H), 6.97-7.09 (m, 2H), 7.34-7.36 (m, 1H).

### Step 3. (R)-tert-butyl 3-(2-(cyclohexenyl)-3-fluorobenzoyl)piperidine-1-carboxylate

A 50-mL, three-necked flask was charged with magnesium turnings (0.56 g, 23.2 mmol) and a small crystal of iodine. The flask was evacuated and refilled with N₂. A solution of 1-bromo-2-(cyclohexenyl)-3-fluorobenzene (4.43 g, 17.4 mmol) in THF (17 mL) was added dropwise. The reaction mixture was stirred and heated under reflux for 2 h and most of magnesium was consumed. The Grignard solution was cooled to rt.

A 100-mL, three-necked flask was charged (R)-tert-butyl 3-(methoxy(methyl)carbamoyl)piperidine-1-carboxylate (3.15 g, 11.6 mmol) and THF (30 mL). The flask was evacuated and refilled with N₂. The mixture was cooled in a dry ice-acetone bath and the Grignard solution prepared above was added. The reaction mixture was allowed to slowly warm to rt while stirring overnight. The mixture was quenched with satd aq NH₄Cl, extracted with EtOAc (3×). The combined organic extracts were dried over Na₂SO₄, filtered and concentrated in vacuo. The residue was purified by silica gel chromatography to afford (R)-tert-butyl 3-(2-(cyclohexenyl)-3-fluorobenzoyl)piperidine-1-carboxylate (1.4 g, 21%). ¹H NMR (400 MHz, CDCl₃): 0.85 (m, 1H), 1.23 (m, 1H), 1.42 (s, 9H), 1.71 (m, 5H), 1.82 (m, 1H), 2.17 (m, 2H), 2.36 (m, 1H), 2.43 (m, 1H), 2.69 (m, 1H), 2.88 (m, 2H), 4.05 (m, 2H), 5.58 (m, 1H), 7.11 (m, 2H), 7.25 (m, 1H).

### Step 4. (R)-tert-butyl 3-(S)-1-(2-(cyclohexenyl)-3-fluorophenyl)-1-hydroxy-5-methoxypentyl)piperidine-1-carboxylate

To a 50-mL, three-necked flask was added (R)-tert-butyl 3-(2-(cyclohexenyl)-3-fluorobenzoyl)piperidine-1-carboxylate (1.4 g, 3.6 mmol) and THF (16 mL). The flask was evacuated and refilled with N₂. The mixture was cooled in a dry ice-acetone bath and 2.0 M 4-methoxybutylmagnesium chloride (20 mL, 40 mmol) was added. The reaction mixture was allowed to slowly warm to rt while stirring overnight. The mixture was quenched with satd aq NH₄Cl and extracted with EtOAc (3×). The combined organic extracts were dried over Na₂SO₄, filtered and concentrated in vacuo. The residue was purified by silica gel chromatography to afford (R)-tert-butyl 3-((S)-1-(2-(cyclohexenyl)-3-fluorophenyl)-1-hydroxy-5-methoxypentyl)piperidine-1-carboxylate (1.3 g, 76%). ¹H NMR (400 MHz, CDCl₃): 0.85 (m, 1H), 1.15-1.39 (m, 4H), 1.45 (d, 9H), 1.79 (m, 2H), 2.17 (m, 2H), 2.24 (m, 2H), 2.52-2.79 (m, 2H), 3.27 (d, 3H), 4.04 (m, 1H), 4.38 (m, 1H), 5.64 (d, 1H), 6.90 (m, 2H), 7.15 (m, 1H).

### Step 5. (S)-1-(2-(cyclohexenyl)-3-fluorophenyl)-5-methoxy-1-((R)-piperidin-3-yl)pentan-1-ol

A solution of (R)-tert-butyl 3-((S)-1-(2-(cyclohexenyl)-3-fluorophenyl)-1-hydroxy-5-methoxypentyl)piperidine-1-carboxylate (680 mg) in 20% TFA/CH₂Cl₂ (30 mL) was stirred at 0° C. for 10 min. Satd aq NaHCO₃ was added to neutralize TFA and the mixture was extracted with CH₂Cl₂ (3×). The combined organic extracts were dried over Na₂SO₄ and evaporated under reduced pressure to afford (S)-1-(2-(cyclohexenyl)-3-fluorophenyl)-5-methoxy-1-((R)-piperidin-3-yl)pentan-1-ol (500 mg, 93%). HPLC analysis of the product indicated the presence of two isomers (1:1).

### Preparation 26

### N-(2-((R)-(6-fluoro-3′-methylbiphenyl-2-yl)((R)-piperidin-3-yl)methoxy)ethyl)acetamide

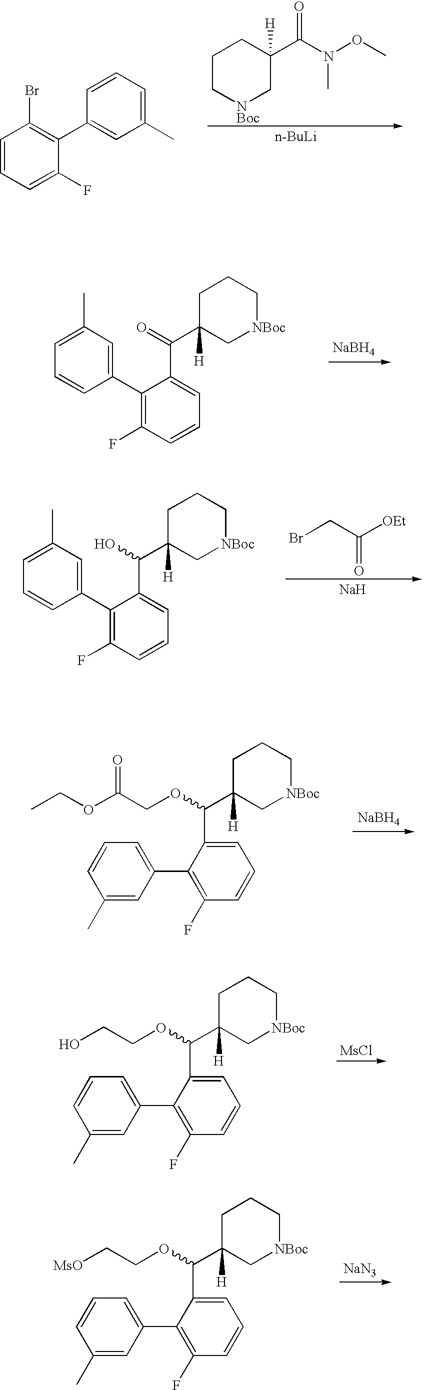

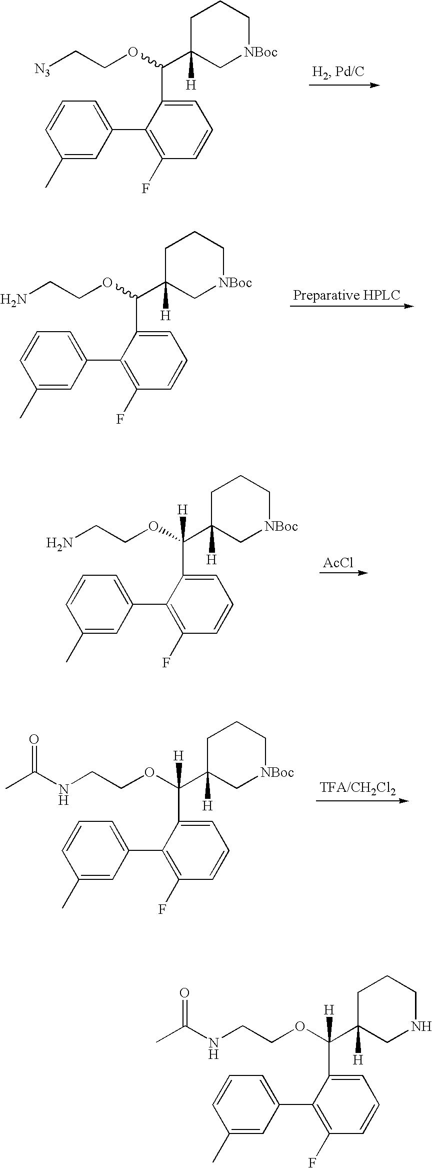

### Step 1. (R)-tert-butyl 3-(6-fluoro-3′-methylbiphenylcarbonyl)piperidine-1-carboxylate

A stirred solution of 6-bromo-2-fluoro-3′-methyl-biphenyl (7 g, 26.4 mmol) in THF (70 mL) under N₂ was cooled to −78° C. and 2.5 M n-BuLi in hexanes (10.56 mL, 26.4 mmol) was added dropwise slowly. The reaction mixture was stirred at −78° C. for 1 h and a solution of the Weinreb amide (R)-tert-butyl 3-(methoxy(methyl)carbamoyl)piperidine-1-carboxylate (7.18 g, 26.4 mmol) in THF (70 mL) was added dropwise slowly. The reaction mixture warmed to rt and stirred overnight. The mixture was quenched with satd aq NH₄Cl and extracted with EtOAc (3×). The combined organic extracts were dried over Na₂SO₄. Solvent removal and flash column chromatography gave (R)-tert-butyl 3-(6-fluoro-3′-methylbiphenylcarbonyl)piperidine-1-carboxylate (4 g, 40%). ¹H NMR (400 MHz, CDCl₃): 0.89 (m, 1H), 1.39 (s, 9H), 1.55 (m, 1H), 1.73 (m, 1H), 2.03 (m, 1H), 2.40 (s, 3H), 2.81 (m, 1H), 3.09 (m, 1H), 3.25 (m, 1H), 3.80 (m, 2H), 3.95 (m, 2H), 7.09-7.41 (m, 7H).

### Step 2. (3R)-tert-butyl 3-((6-fluoro-3′-methylbiphenyl-2-yl)(hydroxy)methyl)piperidine-1-carboxylate

To a solution of (R)-tert-butyl 3-(6-fluoro-3′-methylbiphenylcarbonyl)piperidine-1-carboxylate (3.5 g, 6.29 mmol) in MeOH (50 mL) was added NaBH₄ (0.95 g, 25 mmol) in portions at rt. After addition, the mixture was stirred for 2 h. Tlc showed the starting material had disappeared. The solvent was removed in vacuo to leave a residue which was partitioned between water and EtOAc. The organic layer was washed with H₂O and brine, dried over Na₂SO₄ and evaporated to give (3R)-tert-butyl 3-(6-fluoro-3′-methylbiphenyl-2-yl)(hydroxy)methyl)piperidine-1-carboxylate (3.5 g, 100%), which was used in the next step without purification.

### Step 3. (3R)-tert-butyl 3-((2-ethoxy-2-oxoethoxy)(6-fluoro-3′-methylbiphenyl-2-yl)methyl)piperidine-1-carboxylate

To a suspension of NaH (0.42 g, 17.6 mmol) in THF (50 mL) at 0-5° C. was added dropwise a solution of (3R)-tert-butyl 3-((6-fluoro-3′-methylbiphenyl-2-yl)(hydroxy)methyl)piperidine-1-carboxylate (3.5 g, 8.8 mmol) in THF (30 mL) and the reaction mixture was stirred for 1 h at rt. A solution of ethyl bromoacetate (2.92 g, 17.6 mmol) in THF (30 mL) was added dropwise to the above mixture, and then refluxed for 12 h. Tlc showed the starting material had disappeared. The reaction mixture was poured into satd aq NH₄Cl and extracted with EtOAc. The organic layer was dried over Na₂SO₄, filtered and concentrated in vacuo. The residue was purified by silica gel chromatography to afford (3R)-tert-butyl 3-((2-ethoxy-2-oxoethoxy)(6-fluoro-3′-methylbiphenyl-2-yl)methyl)piperidine-1-carboxylate (1.1 g, 38%). ¹H NMR (400 MHz, CDCl₃): 1.26 (m, 3H), 1.40 (s, 9H), 2.10 (m, 1H), 2.39 (s, 3H), 2.51 (m, 1H), 3.51 (m, 1H), 3.78 (m, 1H), 3.96 (m, 2H), 4.16 (m, 3H), 4.23 (m, 2H), 4.69 (m, 2H), 6.97 (m, 2H), 7.06 (m, 1H), 7.20 (m, 1H), 7.29-7.41 (m, 3H).

### Step 4. (3R)-tert-butyl 3-((6-fluoro-3′-methylbiphenyl-2-yl)(2-hydroxyethoxy)methyl)piperidine-1-carboxylate

To a solution of (3R)-tert-butyl 3-((2-ethoxy-2-oxoethoxy)(6-fluoro-3′-methylbiphenyl-2-yl)methyl)piperidine-1-carboxylate (1.1 g, 2.3 mmol) in EtOH (20 mL) was added NaBH₄ (0.7 g, 18.1 mmol) in portions. After addition, the mixture was stirred at rt overnight. Tlc showed the start material had disappeared. The solvent was removed in vacuo to leave a residue, which was partitioned between water and EtOAc. The organic layer was washed with H₂O and brine, dried over Na₂SO₄, filtered and evaporated to give (3R)-tert-butyl 3-(6-fluoro-3′-methylbiphenyl-2-yl)(2-hydroxyethoxy)methyl)piperidine-1-carboxylate (1 g, 99%) which was used in the next step without purification.

### Step 5. (3R)-tert-butyl 3-(6-fluoro-3′-methylbiphenyl-2-yl)(2-(methanesulfonyloxy)ethoxy)methyl)piperidine-1-carboxylate

To a solution of (3R)-tert-butyl 3-((6-fluoro-3′-methylbiphenyl-2-yl)(2-hydroxyethoxy)methyl)piperidine-1-carboxylate (1 g, 2.3 mmol) in dry CH₂Cl₂ (15 mL) was added Et₃N (0.9 g, 9.0 mmol) at ca 0 to −5° C. A solution of MsCl (0.5 g, 4.5 mmol) in anhydrous CH₂Cl₂ (4 mL) was added dropwise at the same temperature. After addition, the mixture was allowed to warm to rt gradually. Tlc showed the starting material had disappeared. Water was added and the aqueous layer was extracted with CH₂Cl₂. The combined organic extracts were washed with 10% aq citric acid, satd aq NaHCO₃ and brine, dried over Na₂SO₄, filtered and concentrated to give (3R)-tert-butyl 3-(6-fluoro-3′-methylbiphenyl-2-yl)(2-(methanesulfonyloxy)ethoxy)methyl)piperidine-1-carboxylate (1.1 g, yield 94%), which was used in the next step without purification.

### Step 6. (3R)-tert-butyl 3-((2-azidoethoxy)(6-fluoro-3′-methylbiphenyl-2-yl)methyl)piperidine-1-carboxylate

(3R)-tert-butyl 3-((6-fluoro-3′-methylbiphenyl-2-yl)(2-(methanesulfonyloxy)ethoxy)methyl)piperidine-1-carboxylate (1.1 g, 2 mmol) was dissolved in anhydrous DMF (15 mL), solid NaN₃ (280 mg, 4 mmol) was added and the reaction mixture was heated to 80° C. for 5 h. The mixture was cooled to rt and diluted with EtOAc and water. The organic phase was separated, washed with water and dried over MgSO₄. Removal of the solvent gave (3R)-tert-butyl 3-((2-azidoethoxy)(6-fluoro-3′-methylbiphenyl-2-yl)methyl)piperidine-1-carboxylate (0.89 g, yield 90%) which was used in the next step without purification.

### Step 7. (3R)-tert-butyl 3-(2-aminoethoxy)(6-fluoro-3′-methylbiphenyl-2-yl)methyl)piperidine-1-carboxylate

A solution of (3R)-tert-butyl 3-((2-azidoethoxy)(6-fluoro-3′-methylbiphenyl-2-yl)methyl)piperidine-1-carboxylate (0.89 g) in methanol (20 mL) was added to wetted Pd/C (200 mg). After 3 cycles of evacuation and refilling with H₂, a balloon of H₂ was attached to the vessel and the mixture was stirred overnight. The reaction mixture was filtered through a pad of Celite and the solvent was removed to give the crude amine. Purification by preparative HPLC gave (3R)-tert-butyl 3-((R)-(2-aminoethoxy)(6-fluoro-3′-methylbiphenyl-2-yl)methyl)piperidine-1-carboxylate (220 mg, 26%). ¹H NMR (400 MHz, CDCl₃): 1.10 (m, 2H), 1.43 (s, 9H), 1.49 (m, 2H), 1.89 (m, 1H), 2.10 (m, 1H), 2.39 (s, 3H), 3.16 (m, 2H), 3.51 (m, 2H), 4.15 (m, 1H), 6.97 (m, 3H), 7.10 (m, 1H), 7.30-7.48 (m, 3H).

### Step 8. (3R)-tert-butyl 3-((R)-(2-acetamidoethoxy)(6-fluoro-3′-methylbiphenyl-2-yl)methyl)piperidine-1-carboxylate

To a solution of (3R)-tert-butyl 3-((R)-(2-aminoethoxy)(6-fluoro-3′-methylbiphenyl-2-yl)methyl)piperidine-1-carboxylate (86 mg, 0.2 mmol) in anhydrous CH₂Cl₂ (8 mL) was added Et₃N (0.5 ml, 20 mmol). The mixture was cooled with an ice bath and acetyl chloride (15 mg, 0.2 mmol) in CH₂Cl₂ (4 mL) was added. The reaction mixture was stirred at rt for 0.5 h, then washed with water, dried over MgSO₄, filtered and concentrated to give (3R)-tert-butyl 3-((R)-(2-acetamidoethoxy)(6-fluoro-3′-methylbiphenyl-2-yl)methyl)piperidine-1-carboxylate (80 mg, 85%), which was used in the next step without purification.

### Step 9. N-(2-((R)-6-fluoro-3′-methylbiphenyl-2-yl)((R)-piperidin-3-yl)methoxy)ethyl)acetamide

A solution of (3R)-tert-butyl 3-((R)-(2-acetamidoethoxy)(6-fluoro-3′-methylbiphenyl-2-yl)methyl)piperidine-1-carboxylate (80 mg) in 20% TFA/CH₂Cl₂ (5 mL) was stirred at 0° C. for 30 min. The solvent was neutralized by adding satd aq NaHCO₃ and extracted with CH₂Cl₂ (3×). The combined organic extracts were dried over Na₂SO₄ and evaporated to give N-(2-((R)-(6-fluoro-3′-methylbiphenyl-2-yl)((R)-piperidin-3-yl)methoxy)ethyl)acetamide (20 mg, 32%).

The following piperidines were prepared using procedures analogous to those described above:

methyl 2-((R)-(6-fluoro-3′-methylbiphenyl-2-yl)((R)-piperidin-3-yl)methoxy)ethylcarbamate using methyl chloroformate in place of acetyl chloride in Step 8.

3-((R)-6-fluoro-3′-methylbiphenyl-2-yl)(3-methoxypropoxy)methyl)piperidine using 3-methoxypropyl methanesulfonate in Step 3 and eliminating Steps 4-8.

### Preparation 27

### N—((R)-4-(6-fluoro-3′-methylbiphenyl-3-yl)-4-(S)-piperidin-3-yl)butyl)acetamide

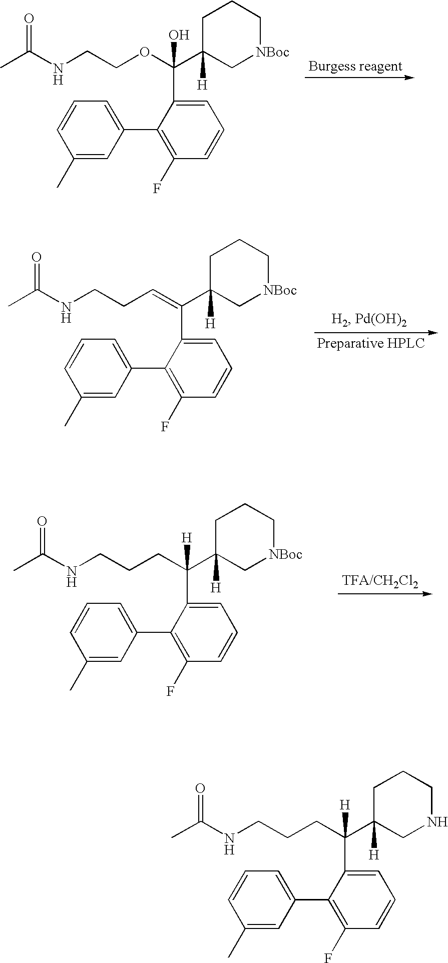

### Step 1. (S)-tert-butyl 3-(4-acetamido-1-(6-fluoro-3′-methylbiphenyl-3-yl)but-1-enyl)piperidine-1-carboxylate

To a solution of (R)-tert-butyl 3-((S)-4-acetamido-1-(6-fluoro-3′-methylbiphenyl-3-yl)-1-hydroxybutyl)piperidine-1-carboxylate (380 mg, 0.76 mmol) in anhydrous toluene (8 mL) was added Burgess reagent (352 mg, 1.47 mmol). The reaction mixture was stirred under reflux overnight. The solvent was removed and the residue was partitioned between EtOAc and H₂O. The aqueous layer was extracted with EtOAc (3×10 mL). The combined organic extracts were washed with brine, dried over Na₂SO₄ and filtered. The filtrate was concentrated in vacuo and the residual oil was purified by preparative tlc to afford (S)-tert-butyl 3-(4-acetamido-1-(6-fluoro-3′-methylbiphenyl-3-yl)but-1-enyl)piperidine-1-carboxylate (110 mg, 30% yield). ¹H NMR (400 MHz, MeOH): 7.33-7.39 (m, 2H), 7.13-7.23 (m, 2H), 6.95-7.03 (m, 3H), 5.29-5.33 (m, 1H), 3.93-4.15 (m, 1H), 3.78-3.91 (m, 1H), 3.00-3.04 (m, 2H), 2.40-2.53 (m, 1H), 2.37 (d, 3H), 1.89 (s, 3H), 1.75 (m, 1H), 1.44-1.62 (m, 4H), 1.41 (s, 9H), 1.16-1.32 (m, 3H), 1.01 (m, 1H). MS (E/Z): 481 (M+H⁺)

### Step 2. (S)-tert-butyl 3-((R)-4-acetamido-1-(6-fluoro-3′-methylbiphenyl-3-yl)butyl)piperidine-1-carboxylate

To a solution of (S)-tert-butyl 3-(4-acetamido-1-(6-fluoro-3′-methylbiphenyl-3-yl)but-1-enyl)piperidine-1-carboxylate (110 mg, 0.85 mmol) in anhydrous MeOH (3 mL) was added anhydrous Pd(OH)₂ (20 mg). The reaction mixture was stirred overnight under a hydrogen atmosphere (monitored by LC-MS) and filtered through a plug of silica. The filtrate was concentrated in vacuo to afford a mixture with two isomers. Purification by preparative HPLC gave (S)-tert-butyl 3-((R)-4-acetamido-1-(6-fluoro-3′-methylbiphenyl-3-yl)butyl)piperidine-1-carboxylate (40 mg, 36% yield). ¹H NMR (400 MHz, MeOH): 7.31-7.37 (m, 2H), 7.20 (d, 2H), 7.13 (d, 2H), 6.97-7.01 (m, 3H), 3.95-4.18 (m, 1H), 3.80-3.92 (m, 1H), 3.03 (m, 2H), 2.61-2.72 (m, 1H), 2.42-2.52 (m, 1H), 2.38 (d, 3H), 1.90 (s, 3H), 1.78 (m, 1H), 1.42-1.65 (m, 4H), 1.43 (s, 9H), 1.15-1.31 (m, 3H), 1.03 (m, 1H). MS (E/Z): 483 (M+H⁺)

### Step 3. N—((R)-4-(6-fluoro-3′-methylbiphenyl-3-yl)-4-((S)-piperidin-3-yl)butyl)acetamide

(S)-tert-butyl 3-((R)-4-acetamido-1-(6-fluoro-3′-methylbiphenyl-3-yl)butyl)piperidine-1-carboxylate (40 mg, 0.083 mmol) was dissolved in a solution of 20% (V/V) TFA/CH₂Cl₂ (3 mL). The reaction mixture was stirred at rt for 1 h (monitored by HPLC) and a solution of satd aq NaHCO₃ was added dropwise to adjust the pH to 7-8. The resulting mixture was extracted with CH₂Cl₂ (3×5 mL) and the combined extracts were washed with brine, dried over Na₂SO₄, and concentrated in vacuo to afford N—((R)-4-(6-fluoro-3′-methylbiphenyl-3-yl)-4-(S)-piperidin-3-yl)butyl)acetamide (30 mg, 94%). MS (E/Z): 383 (M+H⁺).

The following compounds were prepared using procedures analogous to those described above:

methyl (R)-4-(6-fluoro-3′-methylbiphenyl-2-yl)-4-(S)-piperidin-3-yl)butylcarbamate starting with methyl (S)-4-(6-fluoro-3′-methylbiphenyl-2-yl)-4-hydroxy-4-((R)-piperidin-3-yl)butylcarbamate.

### Preparation 28

### N-(2-((S)-(6-fluoro-3′-methylbiphenyl-2-yl)((R)-morpholin-2-yl)methoxy)ethyl)acetamide

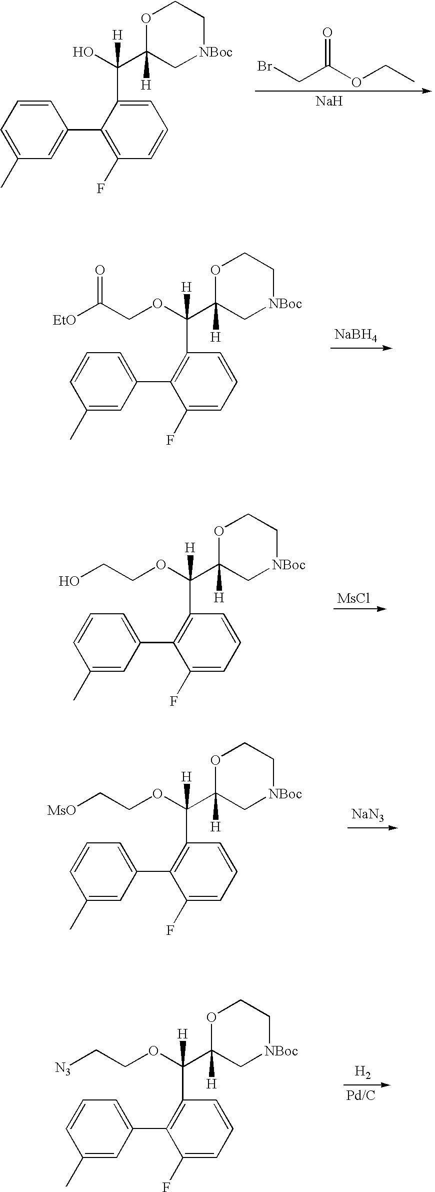

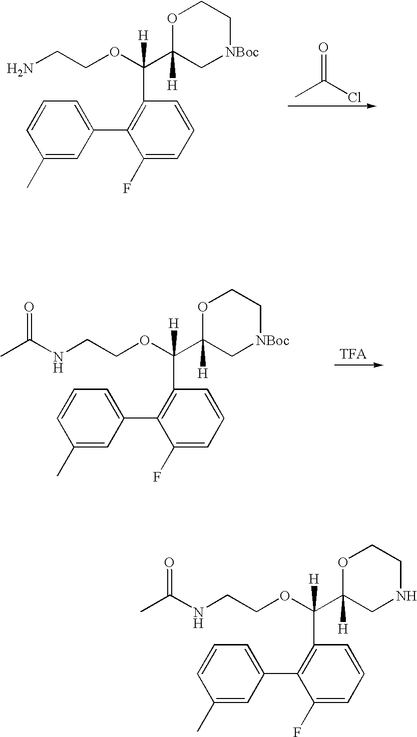

### Step 1. (R)-tert-butyl 2-((S)-(2-ethoxy-2-oxoethoxy)(6-fluoro-3′-methylbiphenyl-2-yl)methyl)morpholine-4-carboxylate

To a slurry of 60% NaH in oil (0.75 g, 18.7 mmol) in THF (30 mL) was added a solution of (R)-tert-butyl 2-((S)-(6-fluoro-3′-methylbiphenyl-2-yl)(hydroxy)methyl)morpholine-4-carboxylate (2.5 g, 6.23 mmol) in THF (20 mL) dropwise at and then the reaction mixture was stirred for about 1 h at rt. A solution of ethyl 3-bromopropionate (1.55 g, 9.35 mmol) in THF (20 mL) was added dropwise while the temperature was maintained at −15 to −5° C. The mixture was allowed to warm slowly to rt and stirred for ˜2 h until the reaction was complete by tlc analysis. The reaction was cooled in an ice bath, quenched with satd aq NH₄Cl (120 mL) and extracted with EtOAc. The combined organic extracts were washed with brine, dried over NaSO₄, concentrated and purified by flash chromatography to afford (R)-tert-butyl 2-(S)-(2-ethoxy-2-oxoethoxy)(6-fluoro-3′-methylbiphenyl-2-yl)methyl)morpholine-4-carboxylate (570 mg, 19%). MS (E/Z): 488 (M+H⁺)

### Step 2. (R)-tert-butyl 2-((S)-(6-fluoro-3′-methylbiphenyl-2-yl)(2-hydroxyethoxy)methyl)morpholine-4-carboxylate

To a solution of (R)-tert-butyl 2-(S)-(2-ethoxy-2-oxoethoxy)(6-fluoro-3′-methylbiphenyl-2-yl)methyl)morpholine-4-carboxylate (570 mg, 1.17 mmol) in CH₃OH (20 mL) at rt, NaBH₄ (355 mg, 9.36 mmol) was added in portions. The mixture was stirred for ˜0.5 h at rt and then evaporated. The residue was partitioned between water and EtOAc. The combined organic layers were washed with brine, dried over anhydrous NaSO₄ and evaporated to give semi-crude (R)-tert-butyl 2-((S)-(6-fluoro-3′-methylbiphenyl-2-yl)(2-hydroxyethoxy)methyl)morpholine-4-carboxylate (498 mg, 96%), which was used in the next step reaction without further purification. MS (E/Z): 446 (M+H⁺)

### Step 3. (R)-tert-butyl 2-((S)-(6-fluoro-3′-methylbiphenyl-2-yl)(2-(methylsulfonyloxy)ethoxy)methyl)morpholine-4-carboxylate

To a solution of (R)-tert-butyl 2-((S)-(6-fluoro-3′-methylbiphenyl-2-yl)(2-hydroxyethoxy)methyl)morpholine-4-carboxylate (498 mg, 1.12 mmol) in dry CH₂Cl₂ (15 mL) was added Et₃N (472 mg, 4.68 mmol) at ˜0 to −5° C. A solution of MsCl (267 mg, 2.34 mmol) in dry CH₂Cl₂ (10 mL) was added dropwise at the same temperature. The mixture was allowed to warm to rt gradually. Tlc showed the stating material had disappeared. Water (10 mL) was added and the aqueous layer was extracted with CH₂Cl₂ (3×20 mL). The combined organic layers were washed with 10% aq citric acid, satd aq NaHCO₃ and brine, dried over Na₂SO₄, filtered and concentrated to afford crude (R)-tert-butyl 2-((S)-(6-fluoro-3′-methylbiphenyl-2-yl)(2-(methylsulfonyloxy)ethoxy)methyl)morpholine-4-carboxylate (554 mg, 95%), which was used in the next step without further purification. MS (E/Z): 524 (M+H⁺)

### Step 4. (R)-tert-butyl 2-((S)-(2-azidoethoxy)(6-fluoro-3′-methylbiphenyl-2-yl)methyl)morpholine-4-carboxylate

To a solution of (R)-tert-butyl 2-((S)-6-fluoro-3′-methylbiphenyl-2-yl)(2-(methylsulfonyloxy)ethoxy)methyl)morpholine-4-carboxylate (554 mg, 1.0 mmol) in anhydrous DMF (18 mL), solid NaN₃ (230 mg, 3.51 mmol) was added and the reaction mixture was heated to 70° C. for overnight. The reaction mixture was cooled to rt and diluted with EtOAc (110 mL), and water (30 ml). The organic phase was washed with water (3×30 mL), dried over Na₂SO₄ and evaporated to give (R)-tert-butyl 2-((S)-(2-azidoethoxy)(6-fluoro-3′-methylbiphenyl-2-yl)methyl)morpholine-4-carboxylate (423 mg, 90%). MS (E/Z): 471 (M+H⁺)

### Step 5. (R)-tert-butyl 2-(S)-(2-aminoethoxy)(6-fluoro-3′-methylbiphenyl-2-yl)methyl)morpholine-4-carboxylate

To a solution of (R)-tert-butyl 2-((S)-(2-azidoethoxy)(6-fluoro-3′-methylbiphenyl-2-yl)methyl)morpholine-4-carboxylate (423 mg, 0.9 mmol) in EtOAc (20 mL) was added wetted Pd/C (42 mg) and the mixture was hydrogenated overnight using a balloon of hydrogen. The mixture was filtered through a pad of Celite and the solvent was removed to give (R)-tert-butyl 2-((S)-(2-aminoethoxy)(6-fluoro-3′-methylbiphenyl-2-yl)methyl)morpholine-4-carboxylate (430 mg, 100%). MS (E/Z): 445 (M+H⁺)

### Step 6. (R)-tert-butyl 2-((S)-(2-acetamidoethoxy)(6-fluoro-3′-methylbiphenyl-2-yl)methyl)morpholine-4-carboxylate

To a round-bottom flask were added (R)-tert-butyl 2-((S)-(2-aminoethoxy)(6-fluoro-3′-methylbiphenyl-2-yl)methyl)morpholine-4-carboxylate (280 mg, 0.63 mmol), triethylamine (0.19 mL, 1.89 mmol) and anhydrous CH₂Cl₂ (15 mL). The mixture was cooled in an ice bath and a solution of acetyl chloride (49.2 mg, 0.045 mL, 0.63 mmol) was added. The reaction mixture was allowed to warm slowly to rt and stirred until the reaction was complete (ca 1˜2 h). The solvent was removed by evaporation, and the residue was purified by preparative tlc to give (R)-tert-butyl 2-(S)-(2-acetamidoethoxy)(6-fluoro-3′-methylbiphenyl-2-yl)methyl)morpholine-4-carboxylate (202 mg, 66%). ¹H NMR (300 MHz, CDCl₃): δ=1.45 (s, 9H), 1.93 (s, 3H), 2.38 (s, 3H), 2.87-3.2 (m, 6H), 3.32-3.92 (m, 5H), 4.28 (d, 1H), 7.01-7.25 (m, 3H), 7.28-7.37 (m, 4H), 9.41-9.54 (s, 1H). MS (E/Z): 487 (M+H⁺)

### Step 7. N-(2-((S)-(6-fluoro-3′-methylbiphenyl-2-yl)((R)-morpholin-2-yl)methoxy)ethyl)acetamide

(R)-tert-butyl 2-((S)-(2-acetamidoethoxy)(6-fluoro-3′-methylbiphenyl-2-yl)methyl)morpholine-4-carboxylate (202 mg, 0.42 mmol) was dissolved in 20% TFA in CH₂Cl₂ (8 mL) and stirred for about 1 h at rt. The mixture was neutralized with satd aq NaHCO₃ and the product was extracted with CH₂Cl₂. The combined organic layers were washed with brine, dried over Na₂SO₄ and concentrated to give N-(2-((S)-(6-fluoro-3′-methylbiphenyl-2-yl)((R)-morpholin-2-yl)methoxy)ethyl)acetamide (130 mg, 82%). ¹H NMR (300 MHz, CDCl₃): δ=1.98 (s, 3H), 2.39 (s, 3H), 2.90-3.3 (m, 6H), 3.31-3.41 (m, 2H), 3.6-4.0 (m, 3H), 4.33 (d, 1H), 6.56-6.57 (s, 1H), 6.97-7.14 (m, 3H), 7.27-7.40 (m, 4H), 9.40-9.55 (s, 1H). MS (E/Z): 387 (M+H⁺).

The following compounds were prepared using procedures analogous to those described above:

methyl 2-((S)-(6-fluoro-3′-methylbiphenyl-2-yl)((R)-morpholin-2-yl)methoxy)ethylcarbamate using methyl chloroformate in place of acetyl chloride in Step 6.

### Preparation 29

### (R)-2-((S)-(6-fluoro-3′-methylbiphenyl-2-yl)(3-methoxypropoxy)methyl)morpholine

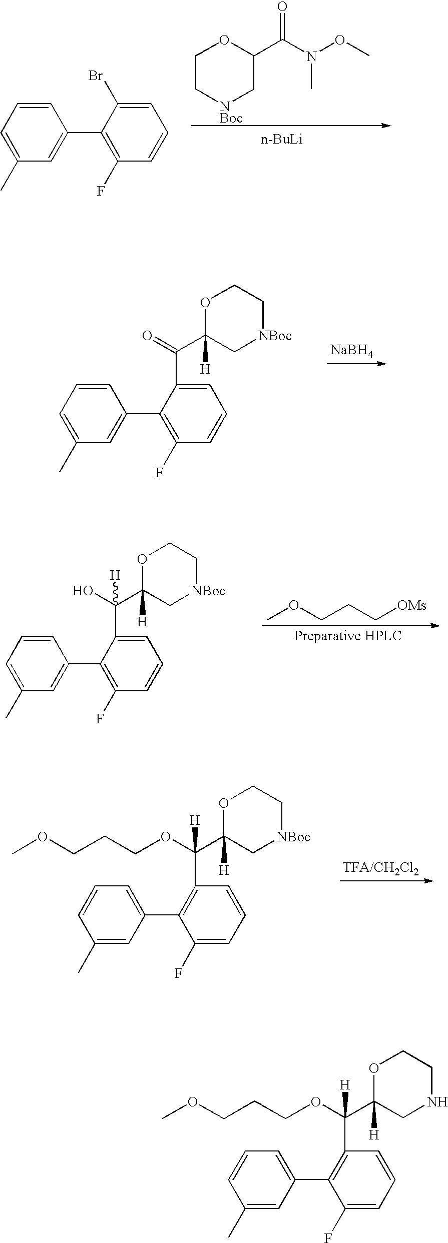

### Step 1. (R)-tert-butyl 2-(6-fluoro-3′-methylbiphenylcarbonyl)morpholine-4-carboxylate

A solution of 2-bromo-6-fluoro-3′-methylbiphenyl (3.4 g, 18.25 mmol) in anhydrous THF (30 ml) under nitrogen was cooled in a dry ice-bath and 2.5M n-BuLi solution (8.76 mL, 18.25 mmol) in hexane was added dropwise slowly. The reaction mixture was stirred at −78° C. for 1 h and a solution of (R)-tert-butyl 2-methoxy(methyl)carbamoyl)morpholine-4-carboxylate (5 g, 18.25 mmol) in anhydrous THF (15 mL) was added dropwise slowly. The reaction mixture was allowed to warm to rt and stirred overnight. The mixture was quenched with sat aq NH₄Cl and extracted with EtOAc (3×30 mL). The combined organic extracts were dried over Na₂SO₄. The solvent was removed and the residue was purified by column chromatograph to afford the (R)-tert-butyl 2-(6-fluoro-3′-methylbiphenylcarbonyl)morpholine-4-carboxylate (3.53 g, 48%). MS (E/Z): 400 (M+H⁺)

### Step 2. (2R)-tert-butyl 2-((6-fluoro-3′-methylbiphenyl-2-yl)(hydroxy)methyl)morpholine-4-carboxylate

To a solution of (R)-tert-butyl 2-(6-fluoro-3′-methylbiphenylcarbonyl)morpholine-4-carboxylate (3.53 g, 8.85 mmol) in EtOH (60 mL), NaBH₄ (1.35 g, 35.4 mmol) was added in portions at rt. The mixture was stirred for about 0.5 h at rt and then evaporated. The residue was partitioned between water and EtOAc. The organic layers were combined and washed with brine, dried over anhydrous Na₂SO₄ and evaporated to give (2R)-tert-butyl 2-(6-fluoro-3′-methylbiphenyl-2-yl)(hydroxy)methyl)morpholine-4-carboxylate (3.40 g, 96%), which was used in the next step reaction without further purification. MS (E/Z): 402 (M+H⁺)

### Step 3. (R)-tert-butyl 2-((S)-(6-fluoro-3′-methylbiphenyl-2-yl)(3-methoxypropoxy)methyl)morpholine-4-carboxylate

To a suspension of NaH (0.3 g, 7.30 mmol) in THF (5 mL) at ˜0 to 5° C. was added dropwise a solution of (2R)-tert-butyl 2-((6-fluoro-3′-methylbiphenyl-2-yl)(hydroxy)methyl)morpholine-4-carboxylate (0.98 g, 2.43 mmol) in THF (15 mL) and the mixture was stirred for 1 h at rt. A solution of 3-methoxypropyl methanesulfonate (2.04 g, 12.16 mmol) in THF (30 mL) was added dropwise and the mixture was stirred under reflux overnight. Tlc indicated the starting material had disappeared. The reaction mixture was poured into satd aq NH₄Cl and extracted with EtOAc. The combined organic extracts were dried over Na₂SO₄, filtered and concentrated in vacuo. The residue was purified by preparative HPLC to afford (R)-tert-butyl 2-((S)-(6-fluoro-3′-methylbiphenyl-2-yl)(3-methoxypropoxy)methyl)morpholine-4-carboxylate (256 mg, 22.3%). ¹H NMR (400 MHz, CDCl₃): 1.44 (s, 9H), 1.66 (m, 5H), 2.39 (s, 3H), 2.64 (m, 1H), 2.84 (m, 1H), 3.13 (m, 1H), 3.41 (m, 2H), 3.76 (m, 2H), 4.05 (m, 1H), 4.21 (m, 1H), 7.06 (m, 2H), 7.19 (m, 2H), 7.34 (m, 3H). MS (E/Z): 507 (M+H⁺)

### Step 4. (R)-2-((S)-(6-fluoro-3′-methylbiphenyl-2-yl)(3-methoxypropoxy)methyl)morpholine

A solution of (R)-tert-butyl 2-((S)-(6-fluoro-3′-methylbiphenyl-2-yl)(3-methoxypropoxy)methyl)morpholine-4-carboxylate (110 mg) in 20% TFA/CH₂Cl₂ (7 mL) was stirred at 0° C. for 1 h. The solvent was neutralized with satd aq NaHCO₃ and extracted with CH₂Cl₂ (3×15 mL). The combined organic extracts were dried over Na₂SO₄ and concentrated in vacuo to afford (R)-2-(S)-(6-fluoro-3′-methylbiphenyl-2-yl)(3-methoxypropoxy)methyl)morpholine (90 mg, 100%). MS (E/Z): 374 (M+H⁺)

### Preparation 30

### (S)-1-(2-tert-butylbenzofuran-7-yl)-5-methoxy-1-((R)-piperidin-3-yl)pentan-1-ol

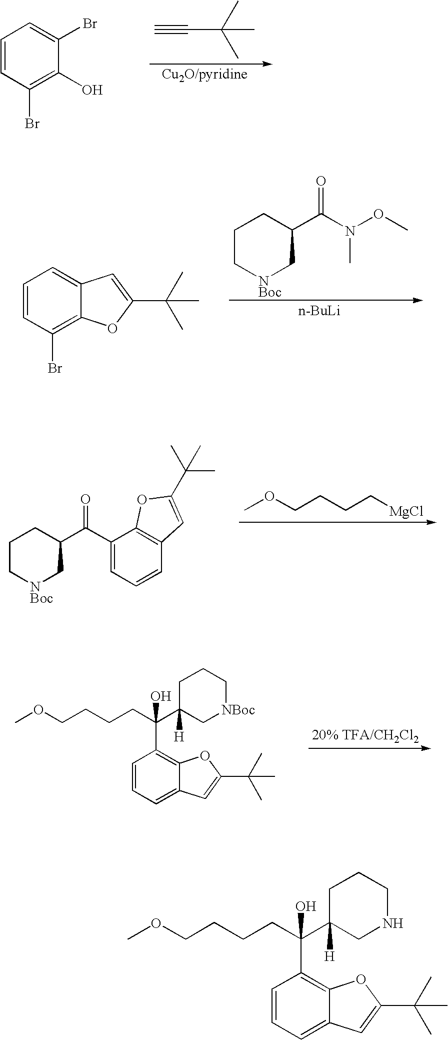

### Step 1. 7-bromo-2-tert-butylbenzofuran

3,3-Dimethylbut-1-yne (1.6 g, 20 mmol) was added to a solution of 2,6-dibromophenol (5.0 g, 20 mmol) and Cu₂O (1.7 g, 12 mmol) in dry pyridine (50 mL) under N₂, then the mixture was heated to about 55° C. and stirred overnight. The mixture was filtered and the filtrate was concentrated to give a residue, which was dissolved in EtOAc. This solution was washed with brine and dried over Na₂SO₄. The solvent was removed and the residue was purified by column chromatography to afford 7-bromo-2-tert-butyl-benzofuran (1.3 g, 26%). ¹H NMR (CDCl₃): 1.40 (S, 9H), 6.41 (s, 1H), 7.04 (t, 1H), 7.38 (d, 1H), 7.42 (d, 1H).

### Step 2. (R)-tert-butyl 3-(2-tert-butylbenzofuran-7-carbonyl)piperidine-1-carboxylate

Under protection of N₂, a solution of 7-bromo-2-tert-butyl-benzofuran (0.5 g, 1.98 mmol) in anhydrous THF (5 mL) was cooled to −78° C. and 2.5 M n-BuLi solution in hexanes (0.87 mL, 2.18 mmol) was added dropwise slowly. The reaction mixture was stirred at −78° C. for 1 h and a solution of (R)-tert-butyl 3-(methoxy(methyl)carbamoyl)piperidine-1-carboxylate (0.65 g, 2.38 mmol) in anhydrous THF (5 mL) was added dropwise slowly. The reaction mixture was warmed to rt and stirred overnight. The mixture was quenched with satd aq NH₄Cl (20 mL) and extracted with EtOAc (3×30 mL). The combined organic extracts were dried over Na₂SO₄. Solvent removal and flash column chromatography afforded (R)-tert-butyl 3-(2-tert-butylbenzofuran-7-carbonyl)piperidine-1-carboxylate (0.41 g, 54%). ¹R NMR (CDCl₃): 7.83 (d, 1H), 7.19 (d, 1H), 7.26 (t, 1H), 6.440 (s, 1H), 4.1 (d, 1H), 3.75 (s, 1H), 2.83 (t, 1H), 2.27 (d, 1H), 1.82 (d, 1H), 1.590 (m, 4H), 1.426 (s, 9H), 1.406 (s, 9H)

### Step 3. (R)-tert-butyl 3-((S)-1-(2-tert-butylbenzofuran-7-yl)-1-hydroxy-5-methoxypentyl)piperidine-1-carboxylate

A 50 mL three-necked flask was charged with (R)-tert-butyl 3-(2-tert-butylbenzofuran-7-carbonyl)piperidine-1-carboxylate (0.41 g, 1.08 mmol) and anhydrous THF (8 mL). The flask was evacuated and refilled with N₂. The mixture was cooled with dry ice-acetone bath and the Grignard reagent derived from 1-chloro-4-methoxy-butane (5.4 mL, 2M) was added. The reaction mixture was allowed to slowly warm to rt while stirring overnight. The mixture was quenched with satd aq NH₄Cl (20 mL) and extracted with EtOAc (3×20 mL). The combined organic extracts were dried over Na₂SO₄ and concentrated in vacuo to afford (R)-tert-butyl 3-((S)-1-(2-tert-butylbenzofuran-7-yl)-1-hydroxy-5-methoxypentyl)piperidine-1-carboxylate (0.5 g, 100%). ¹H NMR: (CDCl3): 1.34 (s, 9H), 1.46 (s, 9H), 1.51 (m, 9H), 2.02 (m, 1H), 2.18 (m, 1H), 2.50 (m, 2H), 2.67 (t, 1H), 3.23 (m, 5H), 3.99 (s, 1H), 4.43 (s, 1H), 6.35 (s, 1H), 7.16 (t, 1H), 7.23 (d, 1H), 7.39 (dd, 1H),

### Step 4. (S)-1-(2-tert-butylbenzofuran-7-yl)-5-methoxy-1-((R)-piperidin-3-yl)pentan-1-ol

(R)-tert-butyl 3-((S)-1-(2-tert-butylbenzofuran-7-yl)-1-hydroxy-5-methoxypentyl)piperidine-1-carboxylate (250 mg, 0.53 mmol) was dissolved in 20% TFA/CH₂Cl₂ (4 mL). The reaction mixture was stirred at rt for 1 h. The mixture was quenched with satd aq NaHCO₃ (15 mL) and extracted with EtOAc (3×10 mL). The combined organic extracts were dried over Na₂SO₄. The filtrate was evaporated to give a residue, which was purified by preparative HPLC to afford pure (S)-1-(2-tert-butylbenzofuran-7-yl)-5-methoxy-1-((R)-piperidin-3-yl)pentan-1-ol (185 mg, 94%). ¹H NMR (CDCl₃): 0.95 (s, 1H), 1.24 (m, 2H), 1.36 (s, 9H), 1.49 (m, 3H), 1.64 (m, 2H), 2.02 (m, 2H), 2.55 (m, 2H), 2.82 (s, 1H), 3.1 (s, 1H), 3.25 (m, 5H), 3.66 (m, 1H), 6.35 (s, 1H), 7.18 (t, 1H), 7.28 (d, 1H), 7.42 (d, 1H), 8.96 (s, 1H), 9.33 (s, 1H)

The following compounds were prepared using procedures analogous to those described above:

(S)-1-(2-isobutylbenzofuran-7-yl)-5-methoxy-1-((R)-piperidin-3-yl)pentan-1-ol using 4-methylpentyne in place of 3,3-dimethylbut-1-yne in Step 1.

### Preparation 31

### (S)-5-methoxy-1-((R)-piperidin-3-yl)-1-(2-(trimethylsilyl)benzofuran-7-yl)pentan-1-ol

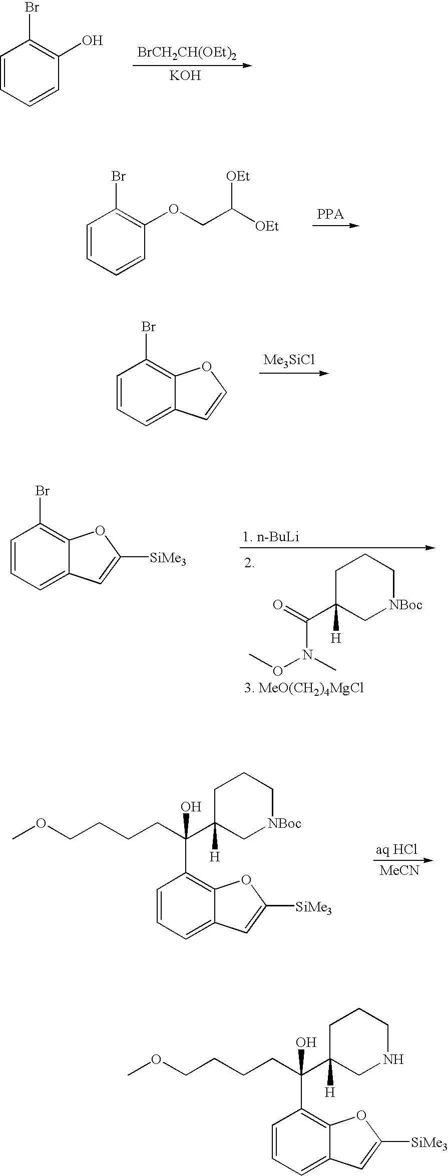

### Step 1. 1-(2,2-diethoxyethoxy)-2-bromobenzene

A solution of KOH pellets (85%, 0.68 g, 10.3 mmol) in water (1.5 mL) was added to 2-bromophenol (1 mL, 8.6 mmol). The mixture was diluted with DMSO (20 mL) and bromoacetaldehyde diethyl acetal (1.43 mL, 9.5 mmol) was added. The mixture was heated at 100° C. for 6 h, cooled to rt, diluted with ether (175 mL), washed with water (3×40 mL) and 5% aq NaOH (40 mL), and dried over MgSO₄. Removal of the solvent left 1-(2,2-diethoxyethoxy)-2-bromobenzene (2.62 g, quant) as an oil.

### Step 2. 7-bromobenzofuran

A stirred mixture of polyphosphoric acid (˜5 g) and chlorobenzene (8 mL) was heated at reflux and a solution of 1-(2,2-diethoxyethoxy)-2-bromobenzene (2.62 g, 9.0 mmol) in chlorobenzene (3 mL) was added dropwise over 10 min. The mixture was heated at reflux for 1.5 h. The mixture was allowed to cool to rt and 1 M aq NaOH (20 mL) was added, followed by ether (175 mL). The mixture was washed with water (2×20 mL) and brine (20 mL), and dried over MgSO₄. Evaporation of the solvent left a residue which was purified by a chromatography on a 140-g silica cartridge eluted with hexanes and a 0-10% EtOAc in hexanes gradient. Appropriate fractions were pooled and concentrated to afford 7-bromobenzofuran (0.65 g, 38% from 2-bromophenol) as a clear colorless oil.

### Step 3. 7-Bromo-2-(trimethylsilyl)benzofuran

A stirred solution of diisopropylamine (0.65 mL, 4.7 mmol) in THF (15 L) was cooled to 5° C. and n-BuLi (2.5 M in hexanes, 1.9 mL, 4.7 mmol) was added dropwise over 5 min. The mixture was stirred at 5° C. for 15 min and cooled to −70° C. Chlorotrimethylsilane (0.59 mL, 4.7 mmol) was added followed by a solution of 7-bromobenzofuran (0.46 g, 2.35 mmol) in THF (5 mL). The mixture was stirred at −70° C. for 1.5 h and poured into sat'd aq NH₄Cl (80 mL). The mixture was diluted with 5% aq HCl (20 mL) and extracted with ether (2×80 mL). The combined ether extracts were washed with sat'd aq NaHCO₃ (50 mL), dried over MgSO₄ and concentrated to leave crude 7-bromo-2-(trimethylsilyl)benzofuran (0.62 g, 98%) as a yellow oil.

### Step 4. (R)-tert-butyl 3-((S)-1-hydroxy-5-methoxy-1-(2-(trimethylsilyl)benzofuran-7-yl)pentyl)piperidine-1-carboxylate

A stirred solution of 7-bromo-2-trimethylsilyl)benzofuran (620 mg, 2.3 mmol) in THF (15 mL) was cooled to −70° C. and n-BuLi (2.5 M in hexanes, 0.85 mL, 2.1 mmol) was added dropwise over 2 min. The mixture was stirred at −70° C. for 15 min and a solution of (R)-tert-butyl 3-(N-methoxy-N-methylcarbamoyl)piperidine-1-carboxylate (341 mg, 1.30 mmol) in THF (5 mL) was added dropwise over 2 min. The mixture was stirred at −70° C. for 1 h, poured into satd aq NaHCO₃ (100 mL) and extracted with ether (2×100 mL). The combined ether extracts were washed with brine (40 mL) and dried over MgSO₄. Removal of the solvent afforded crude (R)-tert-butyl 3-((benzofuran-7-yl)carbonyl)piperidine-1-carboxylate (727 mg) as an oil. This material was dissolved in THF (15 mL) and cooled to −70° C. 4-Methoxybutylmagnesium chloride (1.52 M in THF, 2.0 mL, 3.04 mmol) was added dropwise over 2 min. The mixture was stirred at −70° C. for 2 h and poured into sat'd aq NaHCO₃ (100 mL). The mixture was extracted with ether (2×100 mL) and the combined ether extracts were washed with brine (35 mL) and dried over MgSO₄. Removal of the solvent left an oil which was purified by chromatography on a 40-g silica cartridge eluted with a gradient from 0 to 100% EtOAc in hexanes to afford (R)-tert-butyl 3-((S)-1-hydroxy-5-methoxy-1-(2-(trimethylsilyl)benzofuran-7-yl)pentyl)piperidine-1-carboxylate (240 mg, 39%) as an oil.

### Step 4. (S)-5-methoxy-1-((R)-piperidin-3-yl)-1-(2-(trimethylsilyl)benzofuran-7-yl)pentan-1-ol

(R)-tert-butyl 3-((S)-1-hydroxy-5-methoxy-1-(2-(trimethylsilyl)benzofuran-7-yl)pentyl)piperidine-1-carboxylate (240 mg, 0.49 mmol) was dissolved in MeCN (20 mL) and 5% aq HCl (10 mL) was added. The mixture was stirred at rt for 1 d and solid K₂CO₃ was added. The mixture was diluted with water (40 mL) and extracted with EtOAc (2×100 mL). The combined organic extracts were washed with brine (25 mL), dried over MgSO₄ and concentrated to leave an oil (150 mg) which was purified by reverse phase preparative HPLC to afford (S)-5-methoxy-1-((R)-piperidin-3-yl)-1-(2-(trimethylsilyl)benzofuran-7-yl)pentan-1-ol as its trifluoroactic acid salt (120 mg, 49%) as an oil.

The following piperidines were prepared following procedures analogous to those described above:

(S)-5-methoxy-1-(2-ethylbenzofuran-7-yl)-1-((R)-piperidin-3-yl)pentan-1-ol using 7-bromo-2-ethylbenzofuran and n-BuLi in Step 4.

(S)-5-methoxy-1-((R)-piperidin-3-yl)-1-(2-(trimethylsilyl)benzofuran-4-yl)pentan-1-ol using 2-(trimethylsilyl)-4-bromobenzofuran and n-BuLi in Step 4.

(S)-5-methoxy-1-((R)-piperidin-3-yl)-1-(2-(trimethylsilyl)benzo[b]thiophen-4-yl)pentan-1-ol from 4-bromo-2-(trimethylsilyl)benzothiophene and n-BuLi in Step 4.

### Preparation 32

### 7-bromo-2-ethylbenzofuran

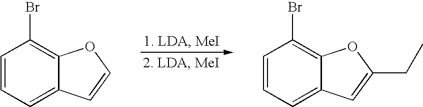

A stirred solution of 7-bromobenzofuran (1.09 g, 5.53 mmol) in dry THF (30 mL) was cooled to −70° C. and 2M LDA in 1:1 THF/heptane (5.5 mL, 11.0 mmol) was added dropwise over 5 min. The mixture was stirred at −70° C. for 20 min and methyl iodide (0.7 mL, 11.2 mmol) was added. The cooling bath was allowed to expire and after 2 h the mixture had warmed to rt. The mixture was poured into satd aq NaHCO₃ (100 mL) and extracted with ether (2×100 mL). The combined ether extracts were washed with 5% aq HCl (50 mL) and dried over MgSO₄. Removal of the solvent left an oil (1.40 g). ¹H NMR showed a mixture of 7-bromobenzofuran, 7-bromo-2-methylbenzofuran and 7-bromo-2-ethylbenzofuran. This material was resubmitted to LDA and MeI under the same conditions to afford, after work up an oil, (1.28 g). Chromatography on a 40-g silica cartridge eluted with hexanes to afforded 7-bromo-2-ethylbenzofuran (0.72 g, 58%, estimated purity ˜80%).

### Preparation 33

### 4-bromo-2-(trimethylsilyl)benzothiophene

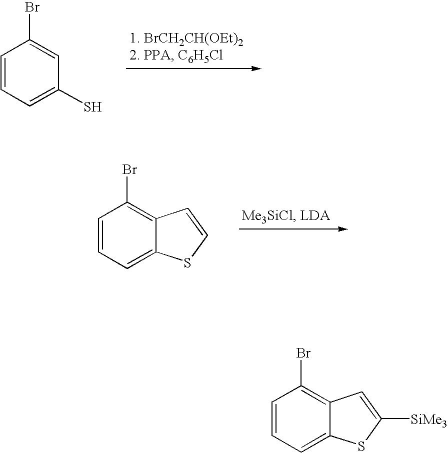

### Step 1. (3-bromophenyl)(2,2-diethoxyethyl)sulfane

To a stirred solution of 3-bromothiophenol (5.0 g, 26 mmol) in DMSO (40 mL) was added a solution of KOH pellets (85% by wt, 2.15 g, 32 mmol) in water (4 mL) followed by bromoacetaldehyde diethyl acetal (4.5 mL, 29 mmol). The mixture was stirred at rt for 5 d, diluted with ether (300 mL) and washed with water (3×100 mL). The combined water washes were extracted with ether (100 mL). The combined ether extracts were washed with brine (100 mL), dried over MgSO₄ and concentrated to afford (3-bromophenyl)(2,2-diethoxyethyl)sulfane (8.23 g, 100%) as a colorless oil.

### Step 2. 4-bromobenzothiophene

A stirred mixture of (3-bromophenyl)(2,2-diethoxyethyl)sulfane (8.23 g, 26 mmol), polyphosphoric acid (20 mL) and chlorobenzene (30 mL) was heated at 130° C. for 1 h. The mixture was allowed to cool to rt and 1 M aq NaOH (100 mL) was added. The mixture was extracted with ether (2×100 mL). The combined ether extracts were washed with water (25 m) and brine (25 mL) and dried over MgSO₄. Removal of the solvent left an oil (29.55 g) which was chromatographed on a 120-g silica cartridge eluted with hexanes. Fractions containing the desired product were concentrated to afford an oil (3.33 g) which resubmitted to chromatography under the same conditions to afford ˜80% pure 4-bromobenzothiophene (1.16 g, 20%).

### Step 3. 4-bromo-2-(trimethylsilyl)benzothiophene

A stirred solution of ˜80% pure 4-bromobenzothiophene (580 mg, 2.7 mmol) and chlorotrimethylsilane (0.70 mL, 5.4 mmol) in dry THF (10 mL) was cooled to −70° C. and 2 M LDA in 1:1 THF/heptane (1.35 mL, 5.4 mmol) was added dropwise over 2 min. The mixture was stirred at −70° C. for 1.5 h and diluted with ether (80 mL) and 5% aq HCl (20 mL). The organic layer was separated, washed with sat'd aq NaHCO₃ (20 mL) and dried over MgSO₄. Removal of the solvent left 4-bromo-2-(trimethylsilyl)benzothiophene (740 mg, 95%) as an amber oil.

4-Bromo-2-(trimethylsilyl)-benzofuran was made following procedures analogous to those described in above, using 3-bromophenol in Step 1.

### Preparation 34

### (S)-1-(2-tert-butylbenzo[d]oxazol-7-yl)-5-methoxy-1-((R)-piperidin-3-yl)pentan-1-ol

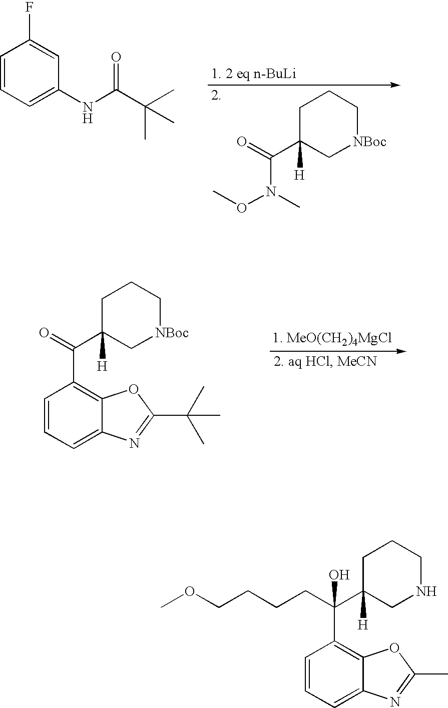

### Step 1. (R)-tert-butyl 3-(2-tert-butylbenzo[d]oxazole-7-carbonyl)piperidine-1-carboxylate

A stirred solution of N-(3-fluorophenyl)pivalamide (317 mg, 1.62 mmol) in dry THF (10 mL) was cooled to −70° C. and 1.6 M n-BuLi in hexanes (2.5 mL, 4.0 mL) was added dropwise over 5 min, such that the temperature remained below −60° C. The cooling bath was allowed to expire and over the course of 1 h the mixture warmed to 0° C. The mixture was stirred at 0 C for 1 h and recooled to −70° C. A solution of (R)-tert-butyl 3-(methoxy(methyl)carbamoyl)piperidine-1-carboxylate (331 mg, 1.22 mmol) in dry THF (5 mL) was added dropwise over 2 min. The mixture was allowed to warm slowly to 0 C as the cooling bath expired and stirred at 0 C for 2 h. The mixture was poured into 5% aq HCl (100 mL) and extracted with ether (2×100 mL). The combined ether extracts were washed with satd aq NaHCO3 (50 mL), dried over MgSO4 and concentrated to afford (R)-tert-butyl 3-(2-tert-butylbenzo[d]oxazole-7-carbonyl)piperidine-1-carboxylate (470 mg, quant) as an oil.

### Step 3. (R)-tert-butyl 3-((S)-1-(2-tert-butylbenzo[d]oxazol-7-yl)-1-hydroxy-5-methoxypentyl)piperidine-1-carboxylate

A stirred solution of (R)-tert-butyl 3-(2-tert-butylbenzo[d]oxazole-7-carbonyl)piperidine-1-carboxylate (470 mg, 1.22 mmol) in dry THF (10 mL) was cooled to −70° C. and 1.63 M 4-methoxybutylmagnesium chloride in THF (2.3 mL, 3.7 mmol) was added dropwise over 2 min. The mixture was stirred at −70° C. for 2 h, allowed to warm to ˜10° C. and poured into satd aq NaHCO₃ (100 mL). The mixture was extracted with ether (2×100 mL). The combined ether extracts were dried over MgSO4 and concentrated to leave an oil (520 mg). Flash chromatography on a 40-g silica cartridge eluted with a 0-100% EtOAc in hexanes to afford (R)-tert-butyl 3-(S)-1-(2-tert-butylbenzo[d]oxazol-7-yl)-1-hydroxy-5-methoxypentyl)piperidine-1-carboxylate (380 mg, 66%) as an oil.

### Step 3. (S)-1-(2-tert-butylbenzo[d]oxazol-7-yl)-5-methoxy-1-((R)-piperidin-3-yl)pentan-1-ol

(R)-tert-butyl 3-((S)-1-(2-tert-butylbenzo[d]oxazol-7-yl)-1-hydroxy-5-methoxypentyl)piperidine-1-carboxylate (380 mg, 0.80 mmol) was dissolved in MeCN (30 mL) and 5% aq HCl (15 mL) was added. The mixture was stirred at rt for 18 h. Additional 5% aq HCl (15 mL) was added and stirring was continued for 1 d. Solid K2CO3 was added and MeCN was evaporated under reduced pressure. The aqueous residue was extracted with CH₂Cl₂ (2×100 mL). The combined organic extracts were dried over Na2SO4 and concentrated. The residue was purified by preparative HPLC to afford (S)-1-(2-tert-butylbenzo[d]oxazol-7-yl)-5-methoxy-1-((R)-piperidin-3-yl)pentan-1-ol as its TFA salt (110 mg, 28%).

### Preparation 35

### (S)-5-methoxy-1-((R)-piperidin-3-yl)-1-(spiro[benzo[d][1,3]dioxole-2,1′-cyclohexane]-4-yl)pentan-

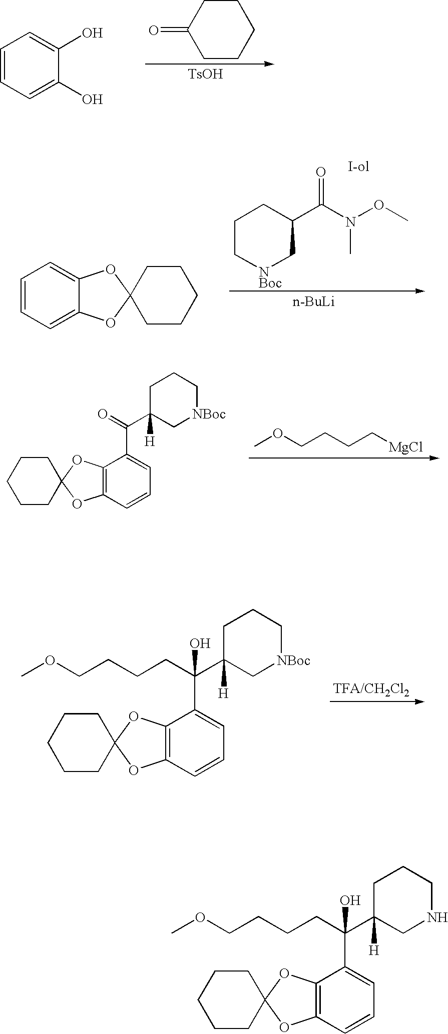

### Step 1. Spiro[benzo[d][1,3]dioxole-2,1′-cyclohexane]

A solution of catechol (6.67 0.06 mol), cyclohexanone (5.88 g, 0.06 mol) and p-toluenesulfonic acid (catalytic amount, ca 2 mg) was refluxed in toluene (60 mL) for 24 h. The water was removed with a Dean-Stark trap. The reaction solution was subsequently washed with 5% aq NaOH (3×60 mL), followed by H₂O (2×10 mL). After the organic layer was dried over Na₂SO₄, it was concentrated under reduced pressure to give a brown oil that solidified on standing. Recrystallization from petroleum ether afforded of spiro[benzo[d][1,3]dioxole-2,1′-cyclohexane] (4.0 g, 35%). ¹H NMR (400 MHz, CDCl₃): 1.4-2.3 (m, 10H), 6.81 (s, 4H)

### Step 2. (R)-tert-butyl 3-(spiro[benzo[d][1,3]dioxole-2,1′-cyclohexane]-4-ylcarbonyl)piperidine-1-carboxylate

To a solution of spiro[benzo[d][1,3]dioxole-2,1′-cyclohexane] (2.5 g, 13 mmol) in anhydrous THF (25 mL) at 0° C. under nitrogen was added dropwise 2.5 M n-BuLi in hexane (5.6 mL, 14 mmol). After addition, the reaction mixture was allowed to warm to rt, stirred for 4 h and cooled to 0° C. A solution of (R)-tert-butyl 3-(methoxy(methyl)carbamoyl)piperidine-1-carboxylate (3 g, 11 mmol) in anhydrous THF (30 mL) was added dropwise and the reaction mixture was allowed to warm to rt and stir overnight. The mixture was quenched with satd aq NH₄Cl (50 mL) and extracted with EtOAc (3×40 mL). The combined organic layers were washed with brine, dried over Na₂SO₄ and concentrated in vacuo. The residue was purified by column chromatography on silica gel to afford (R)-tert-butyl 3-spiro[benzo[d][1,3]dioxole-2,1′-cyclohexane]-4-ylcarbonyl)piperidine-1-carboxylate (0.64 g, 15%). ¹H NMR (400 MHz, CDCl₃): 1.28 (m, 3H), 1.43 (s, 9H), 1.51-1.57 (m, 5H), 1.74-1.80 (m, 4H), 1.91-1.94 (m, 4H), 2.81 (t, 1H), 3.15 (t, 1H), 3.35 (m, 1H), 4.05 (d, 1H), 4.18 (d, 1H), 6.83 (t, 1H), 6.90 (d, 1H), 7.34 (d, 1H). MS (E/Z): 402 (M+H⁺)

### Step 3. (R)-tert-butyl 3-((S)-1-hydroxy-5-methoxy-1-(spiro[benzo[d][1,3]dioxole-2,1′-cyclohexane]-4-yl)pentyl)piperidine-1-carboxylate

To a solution of (R)-tert-butyl 3-spiro[benzo[d][1,3]dioxole-2,1′-cyclohexane]-4-ylcarbonyl)piperidine-1-carboxylate (640 mg, 1.57 mmol) in anhydrous THF (10 mL) at −70° C. under nitrogen was added a solution of 1 M Grignard reagent in THF (16 mL, 16 mmol) dropwise. The mixture was allowed to warm slowly to rt and stirred for 2 h. The mixture was quenched with satd aq NH₄Cl (50 mL) and extracted with EtOAc (3×40 mL). The combined organic layers were washed with brine, dried over Na₂SO₄ and concentrated in vacuo. The residue was purified by chromatography to afford (R)-tert-butyl 3-((S)-1-hydroxy-5-methoxy-1-(spiro[benzo[d][1,3]dioxole-2,1′-cyclohexane]-4-yl)pentyl)piperidine-1-carboxylate (430 mg, 56%). ¹H NMR (400 MHz, CDCl₃): 1.09 (m, 1H), 1.24-1.45 (m, 2H), 1.45 (s, 9H), 1.48-1.57 (m, 5H), 1.61-1.69 (m, 4H), 1.72-97 (m, 6H), 2.07-2.18 (m, 1H), 2.52-2.62 (m, 1H), 2.76 (m, 1H), 3.28-3.33 (m, 4H), 6.83 (t, 1H), 4.05 (m, 1H), 4.12 (m, 1H), 6.66 (m, 1H), 6.76 (m, 2H). MS (E/Z): 490 (M+H⁺)

### Step 4. (S)-5-methoxy-1-((R)-piperidin-3-yl)-1-(spiro[benzo[d][1,3]dioxole-2,1′-cyclohexane]-4-yl)pentan-1-ol

(R)-tert-butyl 3-((S)-1-hydroxy-5-methoxy-1-(spiro[benzo[d][1,3]dioxole-2,1′-cyclohexane]-4-yl)pentyl)piperidine-1-carboxylate (330 mg, 0.67 mmol) was dissolved in 1 N HCl in MeOH (8 mL). The reaction mixture was stirred at rt for 5 h (monitored by HPLC) and a solution of satd aq NaHCO₃ was added dropwise to adjust the pH to 7-8. The solvent was removed and the aqueous residue was extracted with EtOAc (3×10 mL). The combined organic extracts were washed with brine, dried over anhydrous Na₂SO₄ and concentrated in vacuo to afford (S)-5-methoxy-1-((R)-piperidin-3-yl)-1-(spiro[benzo[d][1,3]dioxole-2,1′-cyclohexane]-4-yl)pentan-1-ol (100 mg, 38%). MS (E/Z): 390 (M+H⁺).

The following compounds were prepared following procedures analogous to those described above:

(S)-5-methoxy-1-((R)-piperidin-3-yl)-1-spiro[benzo[d][1,3]dioxole-2,1′-cyclopentane]-4-yl)pentan-1-ol using cyclopentanone in Step 1.

### Preparation 36

### (±)-(1R,2R)-methyl 2-(hydroxymethyl)-1-methylcyclopropanecarboxylate and (±)-(1R,2R)-methyl 2-(hydroxymethyl)-2-methylcyclopropanecarboxylate

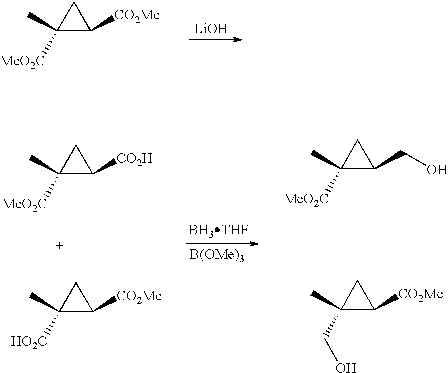

### Step 1. (±)-(1R,2R)-2-(methoxycarbonyl)-2-methylcyclopropanecarboxylic acid and (±)-(1R,2R)-2-(methoxycarbonyl)-1-methylcyclopropanecarboxylic acid

To a stirred solution of (±)-(1R,2R)-dimethyl 1-methylcyclopropane-1,2-dicarboxylate (2.00 g, 11.6 mmol) in THF (5 mL) and MeOH (10 mL) was added a solution of LiOH.H₂O (0.49 g, 11.6 mmol). The mixture was stirred at rt for 2 d and evaporated to leave an aqueous residue which was diluted with satd aq NaHCO₃ (40 mL). The mixture was washed with ether (60 mL) and acidified to ˜pH1 with 5% aq HCl. The mixture was extracted with EtOAc (2×60 mL). The combined EtOAc extracts were dried over MgSO₄ and concentrated to leave a ˜1:1 mixture of (±)-(1R,2R)-2-(methoxycarbonyl)-2-methylcyclopropanecarboxylic acid and (±)-(1R,2R)-2-(methoxycarbonyl)-1-methylcyclopropanecarboxylic acid (1.77 g, 96%).

### Step 2. (±)-(1R,2R)-methyl 2-(hydroxymethyl)-1-methylcyclopropanecarboxylate and (±)-(1R,2R)-methyl 2-(hydroxymethyl)-2-methylcyclopropanecarboxylate

A stirred solution of (±)-(1R,2R)-2-(methoxycarbonyl)-2-methylcyclopropanecarboxylic acid and (±)-(1R,2R)-2-(methoxycarbonyl)-1-methylcyclopropanecarboxylic acid (1.77 g, 11.2 mmol) and trimethyl borate (4 mL, 35.8 mmol) in dry THF (20 mL) was cooled in an ice bath and 1.0 M BH₃ in THF (25 mL, 25 mmol) was added dropwise over 5 min. The ice bath was allowed to melt and stirring was continued at rt for 2 d. The mixture was poured into 5% aq HCl (100 mL) and THF was removed on the rotary evaporator. The aqueous residue was extracted with EtOAc (2×100 mL). The combined EtOAc extracts were washed with satd aq NaHCO3 (50 mL), dried over MgSO4 and concentrated to leave an oil (0.58 g). Chromatography on a 40-g silica cartridge eluted over 20 min with a gradient from 20 to 80% EtOAc in hexanes afforded (±)-(1R,2R)-methyl 2-(hydroxymethyl)-2-methylcyclopropanecarboxylate (99 mg, 6%) followed by (±)-(1R,2R)-methyl 2-(hydroxymethyl)-1-methylcyclopropanecarboxylate (137 mg, 8%).

The following compounds were prepared using procedures analogous to those described above:

(±)-(1R,2R,3R)-methyl 2-(hydroxymethyl)-3-methylcyclopropanecarboxylate using (±)-(1R,2R)-dimethyl 3-methylcyclopropane-1,2-dicarboxylate in Step 1.

### Preparation 37

### 6-((S)-1-hydroxy-5-methoxy-1-((R)-piperidin-3-yl)pentyl)-3′-methylbiphenyl-3-carbonitrile

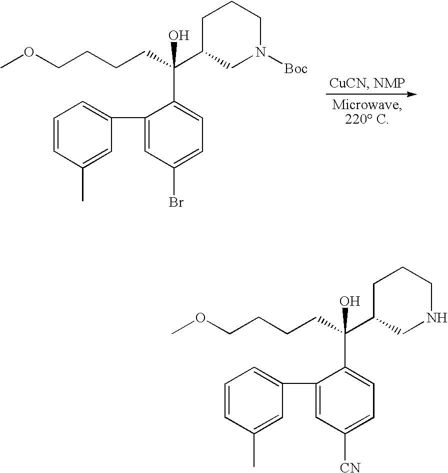

### Step 1. 6-(S)-1-hydroxy-5-methoxy-1-((R)-piperidin-3-yl)pentyl)-3′-methylbiphenyl-3-carbonitrile

To a solution of (R)-tert-butyl 3-((S)-1-(5-bromo-3′-methylbiphenyl-2-yl)-1-hydroxy-5-methoxypentyl)piperidine-1-carboxylate (22.0 mg, 0.040 mmol) in NMP (0.8 mL) was added CuCN (86 mg) and this mixture was heated to 220° C. under microwave for 10 min. The reaction mixture was filtered and purified by preparative HPLC to give 6-((S)-1-hydroxy-5-methoxy-1-((R)-piperidin-3-yl)pentyl)-3′-methylbiphenyl-3-carbonitrile as its TFA salt (10.1 mg, 50%). MS m/z 393 (M+H⁺).

### Preparation 38

### N-{(4S)-4-(6-chloro-3′-ethyl-2-biphenylyl)-4-hydroxy-4-[(3R)-3-piperidinyl]butyl}acetamide

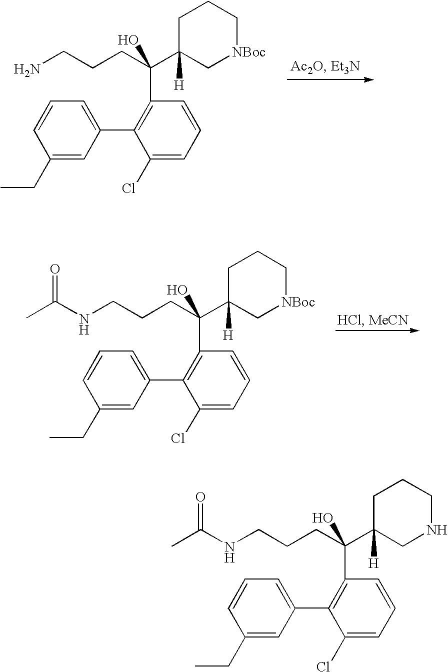

### Step 1. 1,1-dimethylethyl (3R)-3-[(1S)-4-(acetylamino)-1-(6-chloro-3′-ethyl-2-biphenylyl)-1-hydroxybutyl]-1-piperidinecarboxylate

A solution of 1,1-dimethylethyl (3R)-3-[(1S)-4-amino-1-(6-chloro-3′-ethyl-2-biphenylyl)-1-hydroxybutyl]-1-piperidinecarboxylate (75 mg, 0.14 mmol) and Et₃N (0.6 mL, 4.3 mmol) in 2 mL of CH₂Cl₂ at 0° C. was treated with a solution of acetic anhydride (0.047 mL, 0.5 mmol) in 2 mL of CH₂Cl₂ and stirred for 2 h. The mixture was concentrated under reduced pressure and subjected to flash chromatography to provide 1,1-dimethylethyl (3R)-3-[(1S)-4-(acetylamino)-1-(6-chloro-3′-ethyl-2-biphenylyl)-1-hydroxybutyl]-1-piperidinecarboxylate as a colorless oil (53 mg, 73%). MS (m/z) 529.2 (M+H⁺).

### Step 2. N-{(4S)-4-(6-chloro-3′-ethyl-2-biphenylyl)-4-hydroxy-4-[(3R)-3-piperidinyl]butyl}acetamide

A solution of 1,1-dimethylethyl (3R)-3-[(1S)-4-(acetylamino)-1-(6-chloro-3′-ethyl-2-biphenylyl)-1-hydroxybutyl]-1-piperidinecarboxylate (50 mg, 0.095 mmol) in 3 mL of CH₃CN at 25° C. was treated with 3 mL of aqueous 2N HCl. After 24 h, the mixture was concentrated under reduced pressure to provide N-{(4S)-4-(6-chloro-3′-ethyl-2-biphenylyl)-4-hydroxy-4-[(3R)-3-piperidinyl]butyl}acetamide as a white solid (48 mg, quantitative). MS (m/z) 429.2 (M+H⁺).

The following piperidines were prepared following procedures analogous to those described above by substituting the indicated reagent for acetic anhydride in Step 1:

| Structure | Name | Reagent Used in Step 1 |
|---|---|---|
| 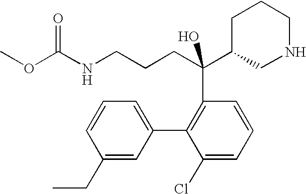 | methyl {(4S)-4-(6-chloro-3′-ethyl-2- biphenylyl)-4-hydroxy-4-[(3R)-3- piperidinyl]butyl}carbamate | methyl chloroformate |
| 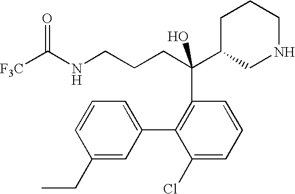 | N-{(4S)-4-(6-chloro-3′-ethyl-2- biphenylyl)-4-hydroxy-4-[(3R)-3- piperidinyl]butyl}-2,2,2-trifluoroacetamide | trifluoroacetic acid |
| 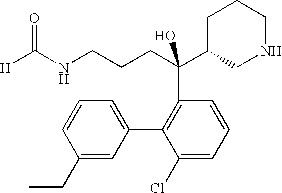 | {(4S)-4-(6-chloro-3′-ethyl-2-biphenylyl)-4- hydroxy-4-[(3R)-3- piperidinyl]butyl}formamide | methyl formate |
| 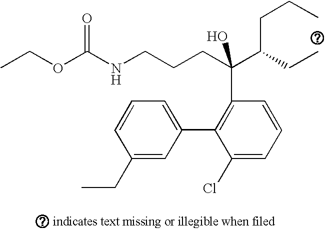 | ethyl {(4S)-4-(6-chloro-3′-ethyl-2- biphenylyl)-4-hydroxy-4-[(3R)-3- piperidinyl]butyl}carbamate | ethyl chloroformate |
| 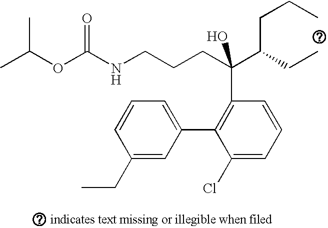 | 1-methylethyl {(4S)-4-(6-chloro-3′-ethyl- 2-biphenylyl)-4-hydroxy-4-[(3R)-3- piperidinyl]butyl}carbamate | isopropyl chloroformate |

### Preparation 39

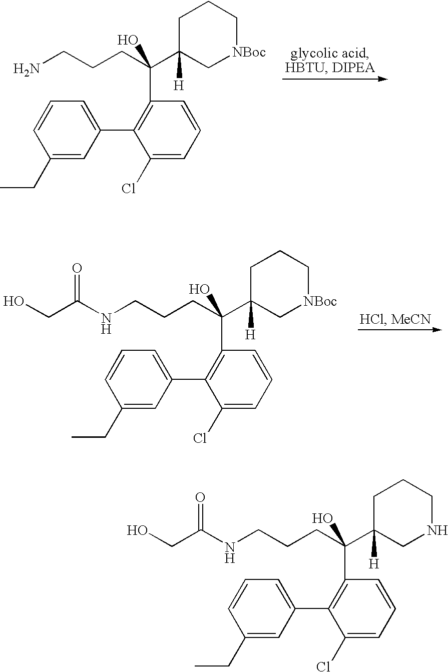

### Step 1. 1,1-dimethylethyl (3R)-3-[(1S)-1-(6-chloro-3′-ethyl-2-biphenylyl)-1-hydroxy-4-[(hydroxyacetyl)amino]butyl]-1-piperidinecarboxylate

A solution of 1,1-dimethylethyl (3R)-3-[(1S)-4-amino-1-(6-chloro-3′-ethyl-2-biphenylyl)-1-hydroxybutyl]-1-piperidinecarboxylate (75 mg, 0.14 mmol) in 0.5 mL of DMF at 25° C. was treated with glycolic acid (13 mg, 0.17 mmol), i-Pr₂NEt (0.122 mL, 0.7 mmol), and HBTU (64 mg, 0.17 mmol). After 24 h, H₂O was added and the mixture was extracted with EtOAc. The organic extracts were washed (1N aq HCl, 1N aq NaOH, H₂O, brine), dried (Na₂SO₄), concentrated under reduced pressure, and subjected to flash chromatography to provide 1,1-dimethylethyl (3R)-3-[(1S)-1-(6-chloro-3′-ethyl-2-biphenylyl)-1-hydroxy-4-[(hydroxyacetyl)amino]butyl]-1-piperidinecarboxylate as a colorless oil (39 mg, 51%). MS (m/z) 567.2 (M+Na⁺).

### Step 2. N-{(4S)-4-(6-chloro-3′-ethyl-2-biphenylyl)-4-hydroxy-4-[(3R)-3-piperidinyl]butyl}-2-hydroxyacetamide

A solution of 1,1-dimethylethyl (3R)-3-[(1S)-1-(6-chloro-3′-ethyl-2-biphenylyl)-1-hydroxy-4-[(hydroxyacetyl)amino]butyl]-1-piperidinecarboxylate (45 mg, 0.08 mmol) in 3 mL of CH₃CN at 25° C. was treated with 3 mL of aq 2N HCl. After 24 h, the mixture was concentrated under reduced pressure to provide N-{(4S)-4-(6-chloro-3′-ethyl-2-biphenylyl)-4-hydroxy-4-[(3R)-3-piperidinyl]butyl}-2-hydroxyacetamide as a white solid (41 mg, quantitative). MS (m/z) 445.2 (M+H⁺).

The following piperidines were prepared following procedures analogous to those described above using the appropriate piperidine and the indicated acid in place of glycolic acid in Step 1:

| Structure | Name | Acid Used in Step 1 |
|---|---|---|
| 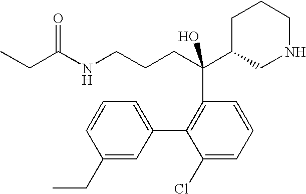 | N-{(4S)-4-(6-chloro-3′-ethyl-2- biphenylyl)-4-hydroxy-4-[(3R)-3- piperidinyl]butyl}propanamide | propionic acid |
| 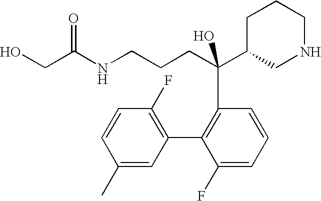 | N-((4S)-4-(2′,6-difluoro-5′- methylbiphenyl-2-yl)-4-hydroxy-4- ((R)-piperidin-3-yl)butyl)-2- hydroxyacetamide | glycolic acid |

### Preparation 40

### 1-bromo-3-chloro-2-[(3-methylphenyl)methyl]benzene

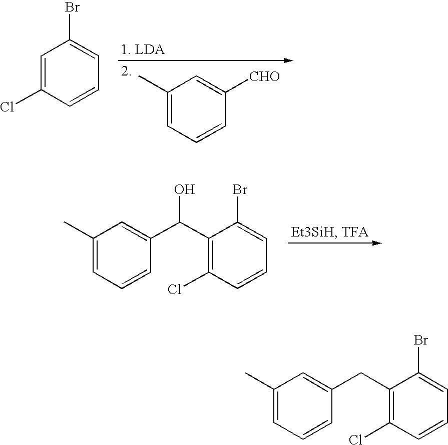

### Step 1. (2-bromo-6-chlorophenyl)(m-tolyl)methanol

To a −78° C. solution of diisopropylamine (9.9 mL, 70 mmol) in anhydrous THF (80 mL) was added dropwise a n-BuLi solution (31.5 mL, 50 mmol, 1.6M hexanes). The reaction was stirred for 20 min at −78° C. and 1-chloro-3-bromobenzene (5.9 mL, 50 mmol) was added. After stirring for 30 min at −78° C., m-tolualdehyde (5.9 mL, 50 mmol) was added. The reaction was gradually allowed to warm to rt and then stirred overnight. The reaction was quenched with the addition of water and then extracted with EtOAc. The organic extracts were dried over MgSO4, filtered and concentrated. The crude residue was purified by flash chromatography on silica gel (ISCO Combiflash, 120 gm column, Hexane/EtOAc 0→10%) and isolated 10.7 g of (2-bromo-6-chlorophenyl)(m-tolyl)methanol.

### Step 2. 1-bromo-3-chloro-2-[(3-methylphenyl)methyl]benzene

(2-bromo-6-chlorophenyl)(m-tolyl)methanol (10.7 g, 34.4 mmol) was dissolved in CH₂Cl₂ (50 mL) and then Et₃SiH (22 mL, 138 mmol) and trifluoroacetic acid (10.6 mL, 138 mmol) were added. After stirring at rt overnight, the reaction was concentrated to remove solvent. The crude residue was purified by flash chromatography on silica gel (ISCO Combiflash, 120 gm column, Hexane/EtOAc 0→10%) and isolated 8.7 g of 1-bromo-3-chloro-2-[(3-methylphenyl)methyl]benzene as a white solid.

1-bromo-3-chloro-2-[(2-methylphenyl)methyl]benzene was prepared using procedures analogous to those described above using o-tolualdehyde in Step 1.

### Preparation 41

### 5-(2-bromo-6-chlorophenyl)-3-methyl-1,2,4-oxadiazole

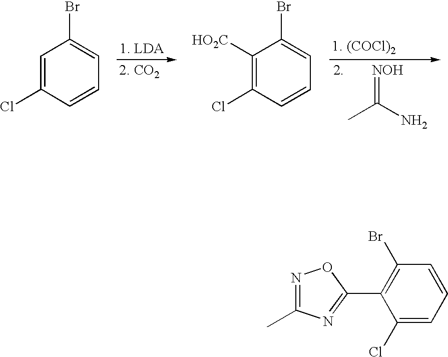

### Step 1. 2-bromo-6-chlorobenzoic acid

To a −78° C. solution of n-BuLi (10 mL, 25 mmol, 2.5M Hexanes) in anhydrous THF (70 mL) was added diisopropylamine (3.5 mL, 25 mmol). After stirring for 15 min, 1-chloro-3-bromobenzene (4.32 g, 25 mmol) was added and stirred for 2 h at −78° C. Dry ice (CO₂) was added and after 15 min a 2N aq HCl solution (100 mL) was added. The reaction mixture was extracted with EtOAc. The product was recrystallized from hexanes and isolated 5 g (85%) of 2-bromo-6-chlorobenzoic acid.

### Step 2. 5-(2-bromo-6-chlorophenyl)-3-methyl-1,2,4-oxadiazole

To a solution of 2-bromo-6-chlorobenzoic acid (1 g, 4.25 mmol) in anhydrous CH₂Cl₂ were added dropwise oxalyl chloride (0.45 mL, 5.1 mmol) and 2-3 drops of DMF. The solution was stirred at rt for 2 h and then the solvent was evaporated. The crude residue was added dropwise to a stirred suspension of the acetamide oxime (315 mg, 4.25 mmol) in pyridine (6 mL). After the addition the mixture was refluxed overnight. The solvent was evaporated and the crude residue purified by flash chromatography to afford 376 mg (32%) of 5-(2-bromo-6-chlorophenyl)-3-methyl-1,2,4-oxadiazole.

### Preparation 42

### 3,5-dimethoxyphenylboronic acid

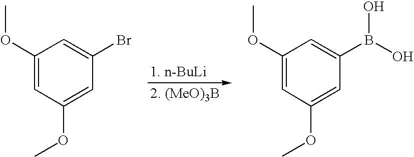

To a solution of 1-bromo-3,5-dimethoxybenzene (5 g, 23 mmol) in THF (100 mL) at −78° C. was added n-Bu-Li (2.5M in hexane, 10 mL, 25 mmol). The mixture was stirred at −78° C. for 30 min and transferred to a solution of B(OCH₃)₃ (3.1 ml) in THF at −78° C. The resulting mixture was warmed up to rt and allowed to stir overnight. The reaction was quenched with 2N aq HCl and extracted with EtOAc. The combined organic extracts were dried over Na₂SO₄ and concentrated. The residue was washed with hexane to give 2.2 g (53% yield) of 3,5-dimethoxyphenylboronic acid as a solid. MS m/z=182.2 (M+H)⁺.

### Preparation 43

### 3-methoxy-5-methylphenylboronic acid

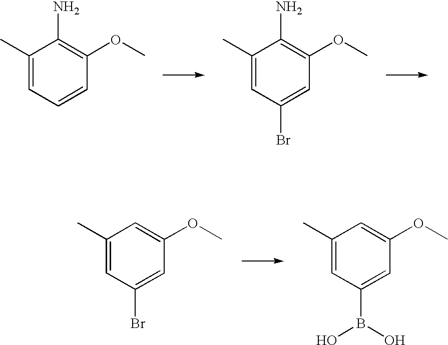

### Step 1. 4-bromo-2-methoxy-6-methylaniline

2-methoxy-6-methylaniline (24.2 g, 182 mmol) was dissolved in MeOH (81 mL) and acetic acid (27 mL) and a solution of bromine (28 g, 182 mmol) in acetic acid (81 mL) was added dropwise. The reaction was allowed to stand at rt for 2 h and concentrated to remove solvents. The crude product was recrystallized from hexanes to give 36 g of 4-bromo-2-methoxy-6-methylaniline as a brown solid.

### Step 2. 1-bromo-3-methoxy-5-methylbenzene

To a cold (0° C.) solution of 4-bromo-2-methoxy-6-methylaniline (36 g, 167 mmol) in a mixture of acetic acid (280 mL), water (120 mL) and concentrated HCl (32 mL) was added dropwise a solution of NaNO₂ (13.8 g, 200 mmol) in water (40 mL). The reaction mixture was stirred for 30 min at 0° C. and 50% aq H₃PO₂ (320 mL) was added. After stirring for 8 h at 0° C., the reaction mixture was allowed to stand at rt for 48 h. The reaction mixture was extracted with EtOAc/Et₂O.

The crude residue was purified by flash chromatography on silica gel (ISCO Combiflash, 330 g column, 100% hexane) to afford 27.5 g of 1-bromo-3-methoxy-5-methylbenzene as a colorless oil.

### Step 3. 3-methoxy-5-methylphenylboronic acid

To a −78° C. solution of 1-bromo-3-methoxy-5-methylbenzene (10 g, 49.8 mmol) in anhydrous THF (200 mL) was added dropwise a n-BuLi solution (37.3 mL, 59.7 mmol, 1.6 M Hexane). After stirring for 30 min at −78° C., trimethyl borate (13.9 mL, 124.3 mmol) was added. The resulting mixture was stirred at −78° C. for 30 min and then warmed to rt and stirred for an additional 60 min. The reaction mixture was poured into an ice/H₂O mixture and acidified with 2N HCl to pH=3. The aqueous solution was extracted with Et₂O. The combined organic extracts were dried over Na₂SO₄, filtered and concentrated in vacuo. The crude residue (13 g) was washed with hexanes. The precipitate was collected and recrystallized from hexanes to give 6.5 g (79%) of 3-methoxy-5-methylphenylboronic acid as a white solid.

### Preparation 44

### 4-((tert-butoxycarbonylamino)methyl)-2-fluorobenzoic acid

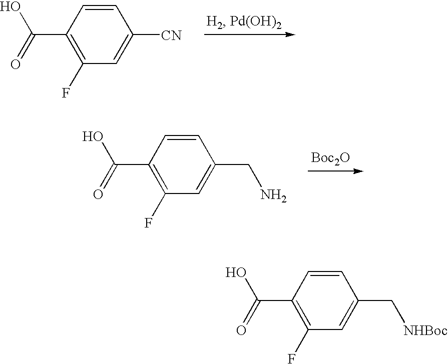

### Step 1. 4-(aminomethyl)-2-fluorobenzoic acid

A solution of 4-cyano-2-fluorobenzoic acid (1.0 g, 6.06 mmol) in 20 mL of MeOH at 25° C. was treated with of 20% Pd(OH)₂/C (300 mg, wet) and stirred overnight under an atmosphere of hydrogen. The reaction mixture was filtered and concentrated under reduced pressure to provide 4-(aminomethyl)-2-fluorobenzoic acid (1.0 g, quantitative).

### Step 2. 4-((tert-butoxycarbonylamino)methyl)-2-fluorobenzoic acid

A solution of 4-aminomethyl)-2-fluorobenzoic acid (1.0 g, 6.0 mmol) in 50 mL of THF at 25° C. was treated with 50 mL of 1N aq NaOH and Boc₂O (1.5 g, 6.9 mmol) and the mixture was stirred overnight before being diluted with the addition of 25 mL of water and 10 mL of brine, acidified slowly to pH 3 using 1N aq HCl, and extracted with EtOAc (3×20 ml). The combined organic extracts were dried (Na₂SO₄) and concentrated under reduced pressure to provide 4-((tert-butoxycarbonylamino)methyl)-2-fluorobenzoic acid.

The following benzoic acids were prepared following procedures analogous to those described above by using the indicated starting material and catalyst in Step 1:

| Structure | Name | Starting Material | Catalyst |
|---|---|---|---|
| 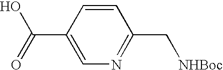 | 6-[({[(1,1- dimethylethyl)oxy]carbonyl}amino)methyl]-3- pyridinecarboxylic acid | 6-cyano-3- pyridinecarboxylic acid | Pd/C |
| 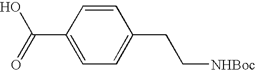 | 4-[2-({[(1,1- dimethylethyl)oxy]carbonyl}amino)ethyl]benzoic acid | 4-(2-aminoethyl)benzoic acid (Step 1 omitted) | Step 1 Omitted |

### Preparation 45

### 4-((tert-butoxycarbonyl(methyl)amino)methyl)benzoic acid

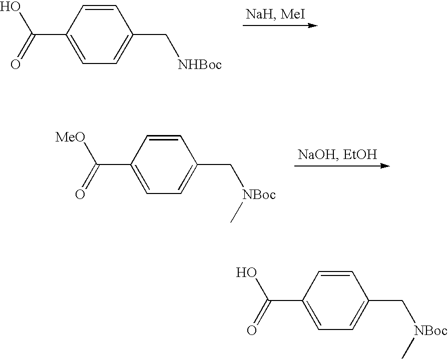

### Step 1. Methyl 4-((tert-butoxycarbonyl(methyl)amino)methyl)benzoate

A solution of 4-((tert-butoxycarbonylamino)methyl)benzoic acid (1.01 g, 4.0 mmol) in 10 mL of DMF at 0° C. was treated with NaH (60% in oil, 400 mg, 10 mmol) and warmed to 25° C. After 10 min, methyl iodide (3 mL) was added and the mixture was stirred at 25° C. for 16 h before being concentrated under reduced pressure. The residue was treated with water (20 mL) and extracted with EtOAc (3×20 mL). The combined organic extracts were washed (brine), dried (Na₂SO₄), concentrated, and subjected to flash chromatography to provide methyl 4-((tert-butoxycarbonyl(methyl)amino)methyl)benzoate as a clear oil (849 mg, 76%). MS (m/z) 280.3 (M+H⁺).

### Step 2. 4-((tert-butoxycarbonyl(methyl)amino)methyl)benzoic acid

A solution of methyl 4-((tert-butoxycarbonyl(methyl)amino)methyl)benzoate (300 mg, 1.08 mmol) in EtOH (10 ml) at 25° C. was treated with aqueous 1N NaOH (2.16 mL, 2.16 mmol) and the mixture was stirred for 16 h before being extracted with EtOAc (2×5 mL). The aqueous layer was acidified by the addition of aqueous 1N HCl and then extracted with EtOAc (3×10 ml). The combined organic extracts were washed (brine), dried (Na₂SO₄), and concentrated to provide 4-((tert-butoxycarbonyl(methyl)amino)methyl)benzoic acid as a white solid (215 mg, 75%). MS (m/z) 266.1 (M+H⁺).

The following benzoic acids were prepared following procedures analogous to those described above by using the indicated starting material and alkylating agent in Step 1:

| | | | Alkylating |
|---|---|---|---|
| Structure | Name | Starting Material | Agent |
| 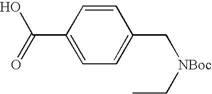 | 4-{[{[(1,1- dimethylethyl)oxy]car- bonyl}(ethyl)amino]methyl}benzoic acid | 4-[({[(1,1- dimethylethyl)oxy]carbonyl}amino)methyl]ben- zoic acid | Ethyl iodide |
| 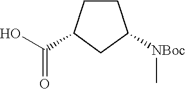 | (1R,3S)-3-[{[(1,1- dimethylethyl)oxy]carbonyl}(methyl)amino]cyclo- pentanecarboxylic acid | (1R,3S)-3-({[(1,1- dimethylethyl)oxy]carbonyl}amino)cyclopentane- carboxylic acid | Methyl iodide |
| 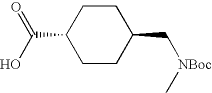 | trans-4-{[{[(1,1- dimethylethyl)oxy]car- bonyl}(methyl)amino]methyl}cyclohexanecar- boxylic acid | trans-4-[({[(1,1- dimethylethyl)oxy]carbonyl}amino)methyl]cyclo- hexanecarboxylic acid | Methyl iodide |

### Preparation 46

### 4-(tert-butoxycarbonyl(isopropyl)amino)methyl)benzoic acid

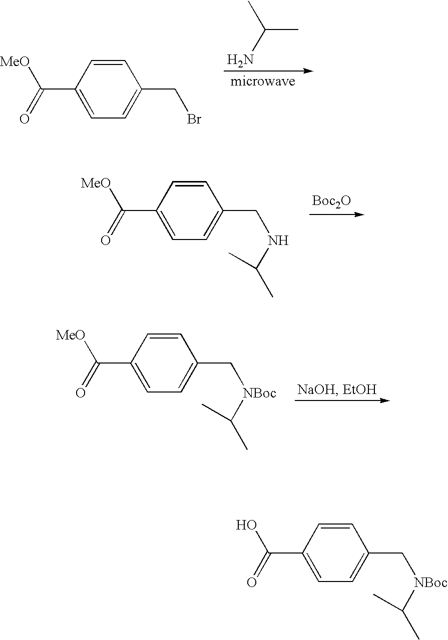

### Step 1. Methyl 4-((isopropylamino)methyl)benzoate

A solution of methyl 4-(bromomethyl)benzoate (1.15 g, 5 mmol) and isopropyl amine (25 mL, 2M in THF, 50 mmol) was heated under microwave irradiation at 100° C. for 10 min before being concentrated under reduced pressure and partitioned between EtOAc and aqueous 1N NaOH. The organic layer was washed (brine), dried (MgSO₄), and concentrated under reduced pressure to provide methyl 4-((isopropylamino)methyl)benzoate as an amber oil (860 mg, 89%). MS (m/z) 208.1 (M+H⁺).

### Step 2. methyl 4-((tert-butoxycarbonyl(isopropyl)amino)methyl)benzoate

A solution of methyl 4-((isopropylamino)methyl)benzoate (1.02 g, 4.92 mmol) in THF (20 ml) at 25° C. was treated with saturated aqueous NaHCO₃ (15 ml) and (Boc)₂O (1.13 g, 5.17 mmol) and stirred for 16 h. The reaction mixture was diluted with EtOAc and the organic phase was separated, washed (H₂O, brine), dried (Na₂SO₄), concentrated under reduced pressure, and subjected to flash chromatography to provide methyl 4-((tert-butoxycarbonyl(isopropyl)amino)methyl)benzoate as a clear oil (1.47 g, 97%). MS (m/z) 308.3 (M+H⁺).

### Step 3. 4-(tert-butoxycarbonyl(isopropyl)amino)methyl)benzoic acid

A solution of methyl 4-((tert-butoxycarbonyl(isopropyl)amino)methyl)benzoate (860 mg, 4.1 mmol) in EtOH (40 mL) at 25° C. was treated with aqueous 1N NaOH (8.2 mL, 8.2 mmol) and the mixture was stirred for 16 h before being extracted with EtOAc (2×20 mL). The aqueous layer was acidified by the addition of aqueous 1N HCl and then extracted with EtOAc (3×40 mL). The combined organic extracts were washed (brine), dried (Na₂SO₄), and concentrated to provide 4-((tert-butoxycarbonyl(isopropyl)amino)methyl)benzoic acid as a white solid (625 mg, 75%). MS (m/z) 238 (M+H⁺-t-Bu).

### Preparation 47

### (R)-1-(6-chloro-3′-ethylbiphenyl-2-yl)-1-((R)-morpholin-2-yl)pent-4-en-1-ol

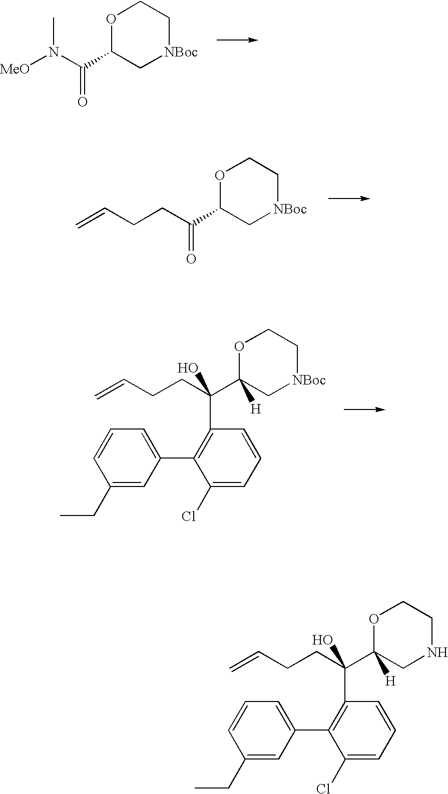

### Step 1. (R)-tert-butyl 2-pent-4-enoylmorpholine-4-carboxylate

To a solution of (R)-tert-butyl 2-methoxy(methyl)carbamoyl)morpholine-4-carboxylate (1.2 g, 4.38 mmol) in 50 mL of THF at −78° C. under a nitrogen atmosphere was slowly added 26 mL (13.3 mmol, 0.5M) of (4-penten-1-yl)magnesium bromide in THF using a syringe. The solution was stirred overnight, allowing it to slowly warm to rt. A saturated solution of NH₄Cl in water (50 mL) was added to the reaction flask. The solution was extracted using EtOAc (3×25 mL). The combined organic extracts were dried over Na₂SO₄ and filtered, followed by concentration under reduced pressure to give 810 mg of (R)-tert-butyl 2-pent-4-enoylmorpholine-4-carboxylate.

### Step 2. (R)-tert-butyl 2-((R)-1-(6-chloro-3′-ethylbiphenyl-2-yl)-1-hydroxypent-4-enyl)morpholine-4-carboxylate

To a solution of 2-bromo-6-chloro-3′-ethylbiphenyl, 2.2 g (7.44 mmol) in 20 mL of THF at −78° C. under a nitrogen atmosphere was slowly added a hexane solution of n-BuLi (3.7 ml, 2.5M) using a syringe. The resulting solution was stirred for 0.5 h. 1,1-dimethylethyl (2R)-2-(4-pentenoyl)-4-morpholinecarboxylate (0.8 g, 2.97 mmol) in 20 mL of THF was slowly added to the above solution using a syringe. The reaction was then allowed to stir and warm to rt overnight. A saturated solution of NH₄Cl in water (50 mL) was added to the reaction flask. The solution was extracted using EtOAc (3×25 mL). The combined organic extracts were dried over Na₂SO₄ and filtered, followed by concentration under reduced pressure. This afforded 550 mg of (R)-tert-butyl 2-((R)-1-(6-chloro-3′-ethylbiphenyl-2-yl)-1-hydroxypent-4-enyl)morpholine-4-carboxylate which was used without purification. LC-MS t_{R}=3.74 min, (m/z) 508.2 (M+H⁺).

### Step 3. (R)-1-(6-chloro-3′-ethylbiphenyl-2-yl)-1-((R)-morpholin-2-yl)pent-4-en-1-ol

To a solution of 1,1-dimethylethyl (2R)-2-[(1R)-1-(6-chloro-3′-ethyl-2-biphenylyl)-1-hydroxy-4-penten-1-yl]-4-morpholinecarboxylate (73 mg, 0.15 mmol) in 5 ml of acetonitrile was added 5 ml of 2N aqueous HCl. The reaction was stirred overnight. It was basified with 10N aqueous NaOH to pH=14 and extracted with DCM (3×10 ml). The combined organic extracts were dried over Na₂SO₄ and filtered, followed by concentration under reduced pressure. This afforded (R)-1-(6-chloro-3′-ethylbiphenyl-2-yl)-1-((R)-morpholin-2-yl)pent-4-en-1-ol which was used without purification.

### Preparation 48

### (R)-1-(6-chloro-3′-ethylbiphenyl-2-yl)-1-((R)-morpholin-2-yl)pent-4-en-1-ol

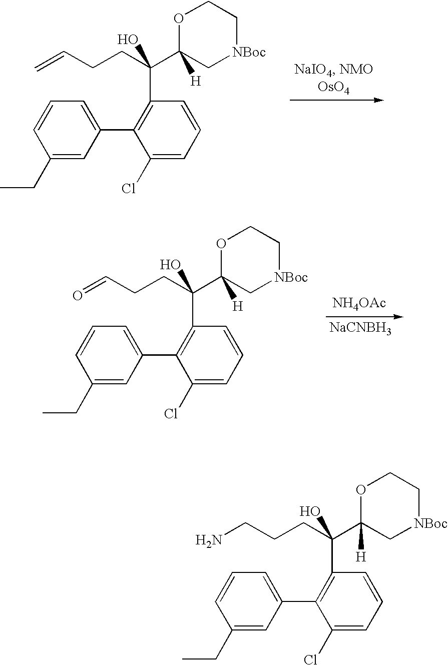

### Step 1. (R)-tert-butyl 2-((R)-1-(6-chloro-3′-ethylbiphenyl-2-yl)-1-hydroxy-4-oxobutyl)morpholine-4-carboxylate

To a solution of (R)-tert-butyl 2-((R)-1-(6-chloro-3′-ethylbiphenyl-2-yl)-1-hydroxypent-4-enyl)morpholine-4-carboxylate (350 mg, 0.72 mmol) in 10 mL of THF and 5 mL of water was added NMO (255 mg, 2.18 mmol), followed by NaIO₄ (310 mg, 1.44 mmol) and a few small crystals of OSO₄. The reaction was stirred overnight. The solution was diluted with 10 mL of water and extracted with CH₂Cl₂ (3×10 ml). The combined organic extracts were dried over Na₂SO₄ and filtered, followed by concentration under reduced pressure. This afforded (R)-tert-butyl 2-((R)-1-(6-chloro-3′-ethylbiphenyl-2-yl)-1-hydroxy-4-oxobutyl)morpholine-4-carboxylate which was used without purification. LC-MS t_{R}=3.36 min, (m/z) 510.2 (M+Na⁺).

### Step 2. (R)-tert-butyl 2-((R)-4-amino-1-(6-chloro-3′-ethylbiphenyl-2-yl)-1-hydroxybutyl)morpholine-4-carboxylate

To a refluxing solution of (R)-tert-butyl 2-((R)-1-(6-chloro-3′-ethylbiphenyl-2-yl)-1-hydroxy-4-oxobutyl)morpholine-4-carboxylate (350 mg, 0.7 mmol) in 20 mL of MeOH was added NH₃.AcOH (550 mg, 7.2 mmol), followed by NaCNBH₃ (135 mg, 2.2 mmol). After a few h at reflux the reaction was cooled to rt and diluted with 20 mL of water. The solution was extracted using EtOAc (3×10 ml). The combined organic extracts were dried over Na₂SO₄ and filtered, followed by concentration under reduced pressure. This afforded (R)-tert-butyl 2-((R)-4-amino-1-(6-chloro-3′-ethylbiphenyl-2-yl)-1-hydroxybutyl)morpholine-4-carboxylate which was used without purification. LC-MS t_{R}=2.56 min, (m/z) 489.2 (M+H⁺).

### Preparation 49

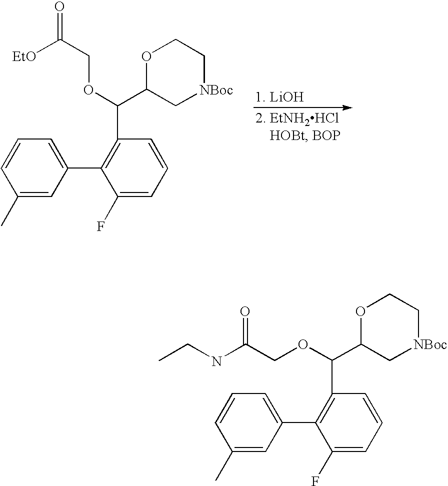

### Step 1. 2-((4-(tert-butoxycarbonyl)morpholin-2-yl)(6-fluoro-3′-methylbiphenyl-2-yl)methoxy)acetic acid

To a solution of tert-butyl 2-(2-ethoxy-2-oxoethoxy)(6-fluoro-3′-methylbiphenyl-2-yl)methyl)morpholine-4-carboxylate (450 mg, 0.924 mmol) in THF (4 mL) were added water (1 mL) and LiOH (78 mg, 1.86 mmol). The reaction mixture was stirred at rt for 3 h. LC-MS indicated complete hydrolysis of the ester. The reaction mixture was concentrated and redissolved in water. The resulting solution was neutralized with 1N aq HCl. The precipitate was collected and dried to give 350 mg of 2-((4-tert-butoxycarbonyl)morpholin-2-yl)(6-fluoro-3′-methylbiphenyl-2-yl)methoxy)acetic acid as a white solid.

### Step 2. tert-butyl 2-(2-(ethylamino)-2-oxoethoxy)(6-fluoro-3′-methylbiphenyl-2-yl)methyl)morpholine-4-carboxylate

To a solution of 2-((4-(tert-butoxycarbonyl)morpholin-2-yl)(6-fluoro-3′-methylbiphenyl-2-yl)methoxy)acetic acid (250 mg, 0.545 mmol), HOBT (147 mg, 1.09 mmol) and BOP (481 mg, 1.09 mmol) in DMF (3 mL) were added i-Pr₂NEt (0.76 mL, 4.36 mmol) and ethylamine hydrochloride (266 mg, 3.27 mmol). The reaction mixture was stirred overnight at rt. LC-MS indicated complete conversion. EtOAc was added to the reaction and then washed with water and brine. The organic phase was dried over MgSO₄, filtered and concentrated to give 0.6 g of an oil. The crude residue was purified by flash chromatography on silica gel [ISCO Combiflash, 40 g column, Hexanes/EtOAc 0%-50%] and isolated 300 mg of tert-butyl 2-(2-ethylamino)-2-oxoethoxy)(6-fluoro-3′-methylbiphenyl-2-yl)methyl)morpholine-4-carboxylate as a white foam.

### Preparation 50

### 1-(3′-ethyl-6-fluorobiphenyl-2-yl)-5-methoxy-1 piperidin-4-yl)pentan-1-ol

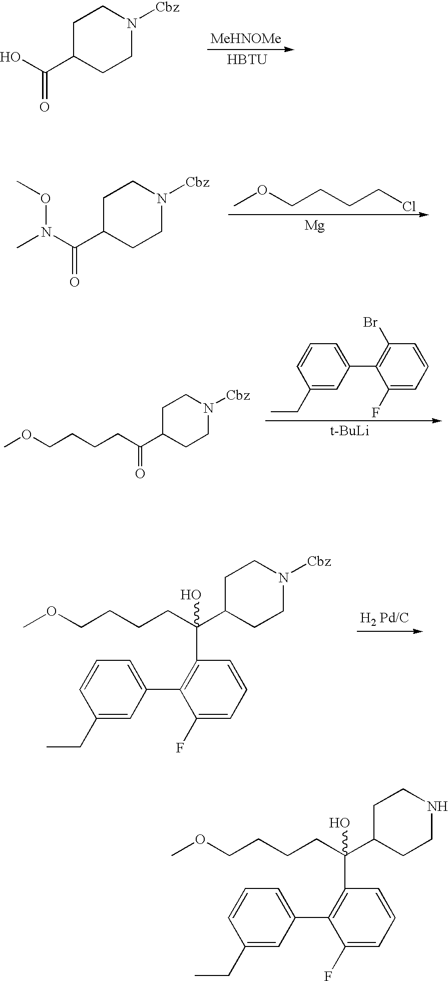

### Step 1. Benzyl 4-methoxy(methyl)carbamoyl)piperidine-1-carboxylate

A solution of 1-(benzyloxycarbonyl)piperidine-4-carboxylic acid (2.1 g, 8.0 mmol) in 20 mL of DMF at 0° C. was treated with N,O-dimethylhydroxylamine hydrochloride (0.84 g, 8.6 mmol), i-Pr₂NEt (7 mL, 40.0 mmol), HBTU (3.3 g, 8.8 mmol), and HOBt (1.2 g, 8.8 mmol) and the mixture was stirred and warmed to 25° C. After 16 h, H₂O (50 mL) was added and the mixture was extracted with EtOAc (3×50 mL). The combined organic extracts were washed (1N HCl, 1N NaOH, H₂O, brine), dried (Na₂SO₄), and concentrated to provide benzyl 4-(methoxy(methyl)carbamoyl)piperidine-1-carboxylate as a yellow oil (2.1 g, 89%).

### Step 2. Benzyl 4-(5-methoxypentanoyl)piperidine-1-carboxylate

A solution of benzyl 4-(methoxy(methyl)carbamoyl)piperidine-1-carboxylate (0.7 g, 2.3 mmol) in 4 mL of THF at −20° C. was treated with a solution of 4-(methyloxy)butyl magnesium chloride (7 mL of 1.28 M in THF, 9.0 mmol) and the mixture was stirred and warmed to 25° C. over 2 h before being quenched with the addition of aqueous 1N HCl and extracted with Et₂O. The combined organic extracts were dried (Na₂SO₄), concentrated, and subjected to flash chromatography to provide benzyl 4-(5-methoxypentanoyl)piperidine-1-carboxylate as a colorless oil (0.67 g, 88%). MS (m/z) 334.2 (M+H⁺).

### Step 3. benzyl 4-(1-(3′-ethyl-6-fluorobiphenyl-2-yl)-1-hydroxy-5-methoxypentyl)piperidine-1-carboxylate

A solution of 2-bromo-3′-ethyl-6-fluorobiphenyl (0.5 mg, 1.8 mmol) in 2 mL of Et₂O at −78° C. was treated with t-BuLi (2.1 mL of 1.7 M in pentane, 3.6 mmol). After 5 min, a solution of benzyl 4-(5-methoxypentanoyl)piperidine-1-carboxylate (0.3 g, 0.9 mmol) in 2 mL of THF was added and the mixture was stirred for 1 h before being quenched with the addition of saturated aqueous NH₄Cl and extracted with Et₂O. The combined organic extracts were dried (Na₂SO₄), concentrated, and subjected to flash chromatography to provide benzyl 4-(1-(3′-ethyl-6-fluorobiphenyl-2-yl)-1-hydroxy-5-methoxypentyl)piperidine-1-carboxylate as a colorless oil (0.15 g, 31%). MS (m/z) 556.2 (M+Na⁺).

### Step 4. 1-(3′-ethyl-6-fluorobiphenyl-2-yl)-5-methoxy-1-(piperidin-4-yl)pentan-1-ol

A solution of benzyl 4-(1-(3′-ethyl-6-fluorobiphenyl-2-yl)-1-hydroxy-5-methoxypentyl)piperidine-1-carboxylate (70 mg, 0.13 mmol) in 2 mL of MeOH at 25° C. was treated with 10% Pd/C (20 mg) and stirred under an atmosphere of hydrogen. After 2 h, the mixture was filtered and concentrated to provide 1-(3′-ethyl-6-fluorobiphenyl-2-yl)-5-methoxy-1-(piperidin-4-yl)pentan-1-ol as a colorless oil (53 mg, quantitative). MS (m/z) 400.3 (M+H⁺).

The following procedures describe preparation of compounds of Formula I.

### Example 1

### (3-(1-(2-(o-Tolyloxy)phenyl)-1-hydroxy-5-methoxypentyl)phenyl) (3-aminopyrrolidin-1-yl)methanone (I-9A)

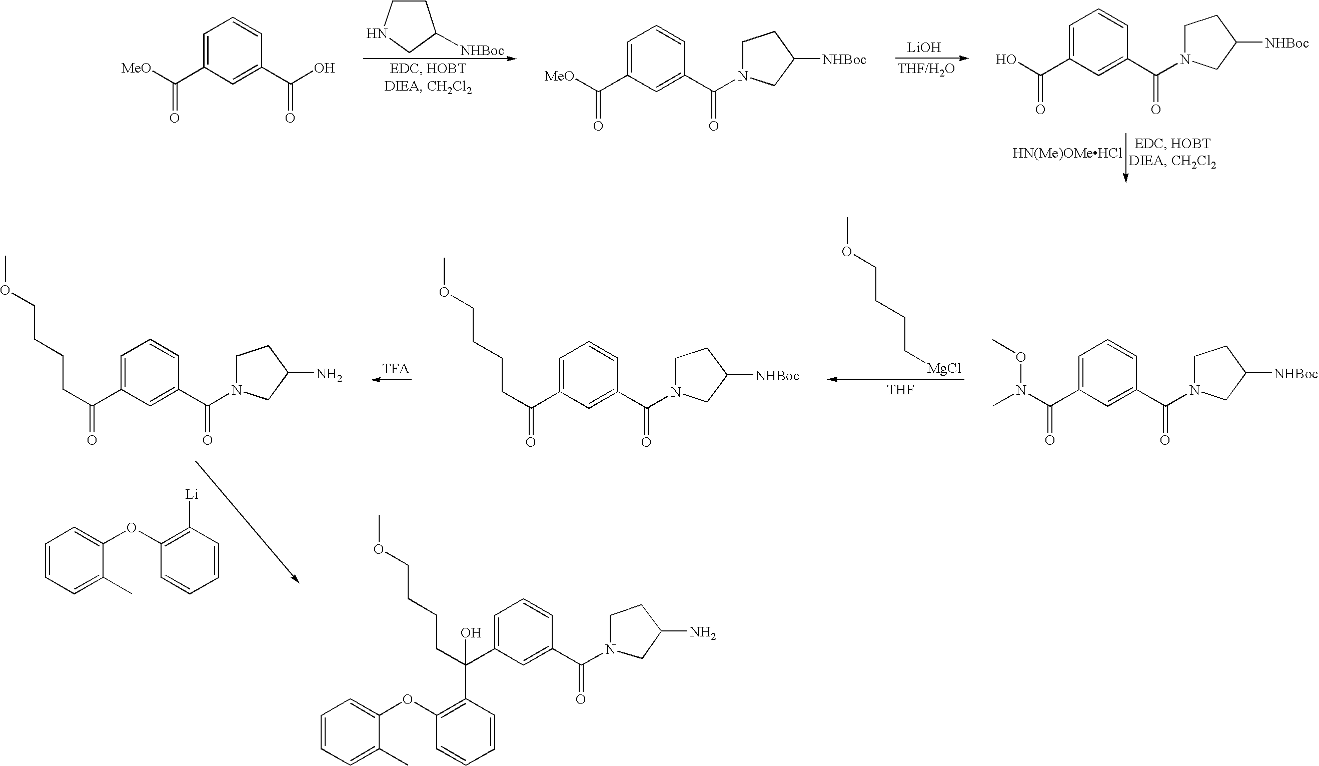

### Step 1. (3-methoxycarbonyl)phenyl)(3-tert-butoxycarbonylamino)pyrrolidin-1-yl)methanone

A mixture of mono-methyl isophthalate (0.5180 g, 2.87 mmol, 1.0 equiv), N-Boc-3-aminopyrrolidine (0.6680 g, 3.58 mmol, 1.24 equiv), EDC.HCl (1.005 g, 5.24 mmol, 1.8 equiv), HOBt (0.610 g, 4.5 mmol, 1.57 equiv), and DIEA (5 mL, 28.7 mmol, 10 equiv) in CH₂Cl₂ (30 mL) was stirred at rt for 24 h. The reaction mixture was diluted with CH₂Cl₂, washed with 1N HCl and 10% Na₂CO₃, and dried over Na₂SO₄. After the solvent was removed, the crude product (0.7387 g, 74%) was used in the next step without further purification.

### Step 2. 3-((3-(tert-butoxycarbonylamino)pyrrolidin-1-yl)carbamoyl)benzoic acid

A mixture of (3-(methoxycarbonyl)phenyl)(3-(tert-butoxycarbonylamino)pyrrolidin-1-yl)methanone (0.7387 g, 2.12 mmol, 1.0 equiv) and lithium hydroxide monohydrate (1.2568 g, 30 mmol, 14 equiv) in THF (50 mL) and H₂O (10 mL) was vigorously stirred at rt for 23 h. The reaction mixture was quenched with 2 N HCl (20 mL), extracted with EtOAc, and dried over Na₂SO₄. The crude product (0.8165 g) was used in the next step without further purification.

### Step 3. (3-(N-methoxy-N-methylcarbamoyl)phenyl)(3-(tert-butoxycarbonylamino)pyrrolidin-1-yl)methanone

A mixture of 3-(3-(tert-butoxycarbonylamino)pyrrolidin-1-yl)carbamoyl)benzoic acid (0.8165 g), NO-dimethylhydroxylamine hydrochloride (0.4736 g, 4.85 mmol, 2.3 equiv), EDC.HCl (0.7416 g, 3.87 mmol, 1.8 equiv), HOBt (0.5763 g, 4.26 mmol, 2.0 equiv), and DIEA (3.5 mL, 20 mmol, 9.5 equiv) in CH₂Cl₂ (20 mL) was stirred at rt for 28 h. The reaction mixture was diluted with brine, extracted three times with CH₂Cl₂ and dried over Na₂SO₄. After the solvent was removed, the crude product (0.2041 g, 25% in two steps) was used in the next step without further purification.

### Step 4. (3-(5-methoxypentanoyl)phenyl)(3-(tert-butoxycarbonylamino)pyrrolidin-1-yl)methanone

To a solution of (3-(N-methoxy-N-methylcarbamoyl)phenyl)(3-(tert-butoxycarbonylamino)pyrrolidin-1-yl)methanone (0.2041 g, 0.54 mmol, 1.0 equiv) in THF (5 mL) was added 1.63 M 4-methoxybutylmagnesium chloride in THF (2 mL, 3.2 mmol, 6 equiv) at 0° C. under N₂. After 1.5 h, the reaction mixture was quenched with 1 N HCl (4 mL), extracted three times with EtOAc and dried over Na₂SO₄. After the solvent was removed, the crude product was used in the next step without further purification.

### Step 5. (3-(5-methoxypentanoyl)phenyl)(3-aminopyrrolidin-1-yl)methanone

A mixture of (3-(5-methoxypentanoyl)phenyl)(3-(tert-butoxycarbonylamino)pyrrolidin-1-yl)methanone and TFA (5 mL) was stirred at rt for 19 h. After the solvent was removed in vacuo, the crude product was purified by reversed-phase HPLC (Phenomenex® Luna 5μ C18(2) 100 A, 250×21.20 mm, 5 micron, 10%→90% CH₃CN/H₂O, 0.1% CF₃COOH over 13 min, flow rate 25 mL/min) to give the trifluoroacetate salt of (3-(5-methoxypentanoyl)phenyl)(3-aminopyrrolidin-1-yl)methanone (0.1020 g, 45% from (3-(5-methoxypentanoyl)phenyl)(3-(tert-butoxycarbonylamino)pyrrolidin-1-yl)methanone).

### Step 6. (3-(1-(2-(o-tolyloxy)phenyl)-1-hydroxy-5-methoxypentyl)phenyl)(3-aminopyrrolidin-1-yl)methanone

To a 50 mL round bottom flask were added 1-(o-tolyloxy)-2-bromobenzene (0.5677 g, 2.15 mmol, 1.0 equiv) and THF (6 mL). The flask was evacuated and refilled with N₂. The mixture was cooled with a dry ice-acetone bath and 1.7 M tert-butyl lithium in pentane (2.6 mL, 4.42 mmol, 2.0 equiv) was added. After 1.5 h, the yellow solution was used in the next step as described below.

To a 100 mL round bottom flask were added the trifluoroacetate salt of (3-(5-methoxypentanoyl)phenyl)(3-aminopyrrolidin-1-yl)methanone (0.0650 g, 0.1553 mmol) and THF (5 mL). The flask was evacuated and refilled with N₂. The mixture was cooled with a dry ice-acetone bath and the yellow solution of 2-(o-tolyloxy)phenyl lithium in THF, prepared as described above, was added via a cannula. The reaction mixture was allowed to slowly warm to −55° C. while stirring overnight (15 h). The mixture was quenched with 10% Na₂CO₃ (2 mL), extracted three times with CH₂Cl₂, and dried over Na₂SO₄. The crude product was purified by reversed-phase HPLC (Phenomenex® Luna 5μ C18(2) 10 A, 250×21.20 mm, 5 micron, 10%→90% CH₃CN/H₂O, 0.1% CF₃COOH over 13 min, flow rate 25 mL/min) to give the trifluoroacetate salt of (3-(1-(2-o-tolyloxy)phenyl)-1-hydroxy-5-methoxypentyl)phenyl)(3-aminopyrrolidin-1-yl)methanone (I-9A, 0.0214 g, 23%). LC-MS (3 min) t_{R}=1.38 min, m/z 511 (M+Na⁺), 489 (M+H⁺), 471; ¹H NMR (400 MHz, CD₃OD) □ 7.83-7.78 (m, 1H), 7.55-6.84 (m, 9H), 6.32 (d, J=7.6 Hz, 1H), 6.16 (m, 1H), 3.84-3.48 (m, 4H), 3.26 (t, J=6.4 Hz, 2H), 3.17 (s, 3H), 2.71-2.62 (m, 1H), 2.24-2.17 (m, 2H), 2.08-2.02 (m, 2H), 1.77 (s, 3H), 1.53-1.37 (m, 3H), 1.19-1.12 (m, 1H).

### Example 2

The following compound was prepared using the procedure described in Example 1: (3-(1-(2-o-tolyloxy)phenyl)-1-hydroxy-5-methoxypentyl)phenyl)((3R,4S)-3-amino-4-hydroxypyrrolidin-1-yl)methanone (I-36A) using (3R,4S)-3-(tert-butoxycarbonylamino)-4-(tert-butyldimethylsilyloxy)pyrrolidine in Step 1.

### Example 3

### ((1S,3R,4S)-3-amino-4-hydroxycyclopentyl)((R)-3-((S)-1-hydroxy-5-methoxy-1-(2-(2,2-(dimethyl)propoxy)phenyl)pentyl)piperidin-1-yl)methanone (I-16A)

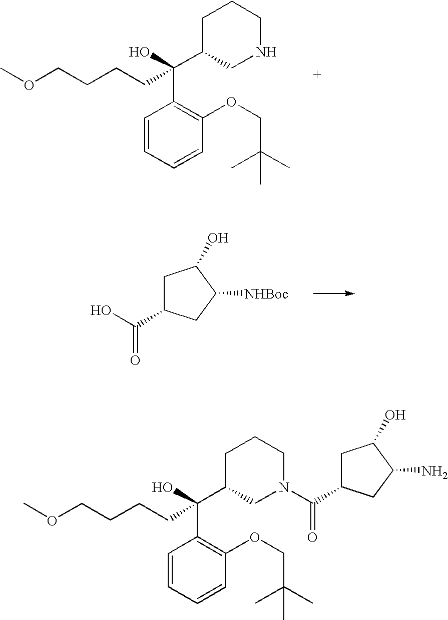

### Step 1. ((1S,3R,4S)-3-(tert-butoxycarbonylamino)-4-hydroxycyclopentyl)((R)-3-((S)-1-hydroxy-5-methoxy-1-(2-(2,2-(dimethyl)propoxy)phenyl)pentyl)piperidin-1-yl)methanone

To a stirred solution of ((S)-5-methoxy-1-(2-(2,2-(dimethyl)propoxy)phenyl)-1-(R)-piperidin-3-yl)pentan-1-ol hydrochloride (10 mg, 0.03 mmol), (1S,3S,4R)-3-hydroxy-4-tert-butoxycarbonylamino)cyclopentane-1-carboxylic acid (7 mg, 0.02 mmol) and DIEA (0.10 mL, 0.54 mmol) in DMF (1 mL) was added HBTU (12 mg, 0.032 mmol). The mixture was stirred for 1 h at rt, the solvent was removed and the residue was purified by preparative HPLC to afford ((1S,3R,4S)-3-(tert-butoxycarbonylamino)-4-hydroxycyclopentyl)((R)-3-((S)-1-hydroxy-5-methoxy-1-(2-(2,2-(dimethyl)propoxy)phenyl)pentyl)piperidin-1-yl)methanone.

### Step 2. ((1S,3R,4S)-3-amino-4-hydroxycyclopentyl)((R)-3-((S)-hydroxy-5-methoxy-1-(2-(2,2-(dimethyl)propoxy)phenyl)pentyl)piperidin-1-yl)methanone

A solution of ((1S,3R,4S)-3-(tert-butoxycarbonylamino)-4-hydroxycyclopentyl)((R)-3-(S)-1-hydroxy-5-methoxy-1-(2-(2,2-(dimethyl)propoxy)phenyl)pentyl)piperidin-1-yl)methanone in MeCN (3 mL) was treated with 2M aq HCl (3 mL) and the mixture was stirred at rt overnight. The solvent was evaporated and the crude mixture purified by preparative HPLC to give ((1S,3R,4S)-3-amino-4-hydroxycyclopentyl)((R)-3-(1-hydroxy-5-methoxy-1-(2-(2,2-(dimethyl)propoxy)phenyl)pentyl)piperidin-1-yl)methanone triflate (I-16A). LC-MS (3 min) m/z 491 (M+H⁺).

### Example 4

### ((1R,3S)-3-Aminocyclopentyl)((R)-3-((S)-1-hydroxy-5-methoxy-1-(2-phenoxy phenyl)pentyl)piperidin-1-yl)methanone (I-4A)

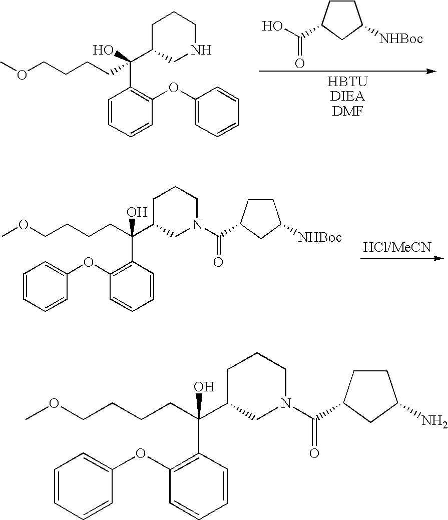

### Step 1. ((1R,3S)-3-(tert-butoxycarbonylamino)cyclopentyl)((R)-3-(S)-1-hydroxy-5-methoxy-1-(2-phenoxyphenyl)pentyl)piperidin-1-yl)methanone

To a solution of (S)-5-methoxy-1-(2-phenoxyphenyl)-1-((R)-piperidin-3-yl)pentan-1-ol (18.5 mg, 0.05 mmol) and (1R,3S)-3-(t-butoxycarbonylamino)cyclopentanecarboxylic acid (12.1 mg, 0.05 mmol) in DMF (0.5 mL) were added DIEA (26 μL. 0.15 mmol), HBTU (19.0 mg, 0.05 mmol), and HOBt (6.8 mg, 0.05 mmol). The resulting solution was stirred at rt for 20 min. Preparative HPLC gave ((1R,3S)-3-(t-butoxycarbonylamino)cyclopentyl)((R)-3-(S)-1-hydroxy-5-methoxy-1-(2-phenoxyphenyl)pentyl)piperidin-1-yl)methanone (19.5 mg, 67%) as a oil. LC-MS (3 min) m/z 581 (M+H).

### Step 2. ((1R,3S)-3-Aminocyclopentyl)((R)-3-((S)-1-hydroxy-5-methoxy-1-(2-phenoxy phenyl)pentyl)piperidin-1-yl)methanone

To a stirred solution of ((1R,3S)-3-(t-butoxycarbonylamino)cyclopentyl)((R)-3-((S) 1-hydroxy-5-methoxy-1-(2-phenoxyphenyl)pentyl)piperidin-1-yl)methanone (19.5 mg) in MeCN (2 mL) was added 5% aq HCl (2 mL). The resulting solution was stirred at rt until no starting material remained (˜16 h), basified to pH=10 with 10 N aq NaOH, and evaporated under reduced pressure to remove MeCN. The aq layer was extracted with CH₂Cl₂ (4×10 mL). The combined organic layers were washed with brine and dried over Na₂SO₄. The crude product was purified by preparative HPLC to give ((1R,3S)-3-aminocyclopentyl)((R)-3-(S)-1-hydroxy-5-methoxy-1-(2-phenoxyphenyl)pentyl)piperidin-1-yl)methanone (I-4A, 17.4 mg) as its TFA salt. ¹H NMR (400 MHz, CD₃OD): 7.64 (m, 1H), 7.38 (m, 2H), 7.08-7.24 (m, 3H), 6.92 (m, 2H), 6.80 (two d, 1H), 4.44, 4.86 (m, 1H), 3.96, 4.26 (m, 1H), 3.68 (m, 1H), 3.36, 3.44 (m, 1H), 3.28 (t, 2H), 3.24 (s, 3H), 2.94, 3.14 (m, 1H), 2.63 (m, 1H), 2.40 (m, 1H), 1.8-2.2 (m, 6H), 1.0-1.8 (m, 8H), 0.92 (m, 1H); LC-MS (3 min) m/z 481 (M+H⁺).

### Example 5

The compounds below were prepared by coupling the appropriate piperidines and Boc protected amino acids followed by deprotection according to the procedures described in Examples 3 and 4: I-1A, I-3A, I-3B, I-4B, I-5A, I-10A, I-10B, I-11A, I-12A, I-12B, I-13A, I-17A, I-17B, I-17C, I-18A, I-19A, I-20A, I-25A, I-25B, I-26A, I-27A, I-27Ba, I-28A, I-29A, I-33A, I-37A, I-37B, I-41A, I-41B, I-43A, 144A, 146A, 147A, I-47B, I-48A, I-49A, I-50A, I-51A, I-52A, I-53A, I-55A, I-56A, I-59A, I-60A, I-61A, I-63A, I-64A, I-65A, I-66A, I-67A, I-68A, I-69A, I-71A, I-74A, I-74B^{a}, I-78A, I-81A, I-82A, I-83A, I-84A, I-85A, I-89A, I-90A, I-92A, I-93A, I-97A, I-98A, I-99A, I-100A, I-101A, I-102A, I-103A, I-104A, I-105A, I-106A, I-115A, I-116A, I-117A, I-120A, I-120B, I-121A, I-122A, I-123A, I-124A, I-125A, I-126A, I-130A, I-131A, I-132A, I-137A, I-140A, I-141A, I-146A, I-150A, I-153A, I-153B^{a}, I-154A, I-155A, I-158A, I-159A, I-163A, I-164A, I-167A, I-169A, I-170A, I-173A, I-174A, I-175A, I-177A, I-179A, I-180A, I-182A, I-183A, I-184A, I-185A, I-186A, I-189A, I-189B^{a}, I-190A, I-191A, I-192A, I-193A, I-193B^{a}, I-194A, I-195A, I-196A, I-197A, I-198A, I-199A, I-200A, I-201A, I-201B^{a}, I-202A, I-203A, I-204A, I-205A, I-205B^{a}, I-206A, I-207A, I-208A, I-209A^{b}, I-210A^{b}, I-211A, I-212A, I-213A, I-214A, I-215A, I-217A, I-218A, I-219B, I-219A, I-220A, I-223A, I-225A, I-228A, I-231A, I-232A, I-233B, I-233A, I-234B, I-234A, I-235A, I-236A, I-237A, I-238A, I-246A, I-251A, I-252A, I-265A, I-265B, I-270A, I-273A, I-279A, I-280A, I-298A, I-320A, I-323A, I-330A, I-331A, I-332A, I-333A. ^{a} Minor isomer isolated by chromatography^{b} HCl in MeOH was used in Step 2 in place of 5% aq HCl/MeCN

### Example 6

### ((R)-3-((S)-1-(2-(o-tolyloxy)phenyl)-1-hydroxy-5-methoxypentyl)piperidin-1-yl)((3S*,4R*)-3-amino-4-hydroxycyclohexyl)methanone (I-62B)

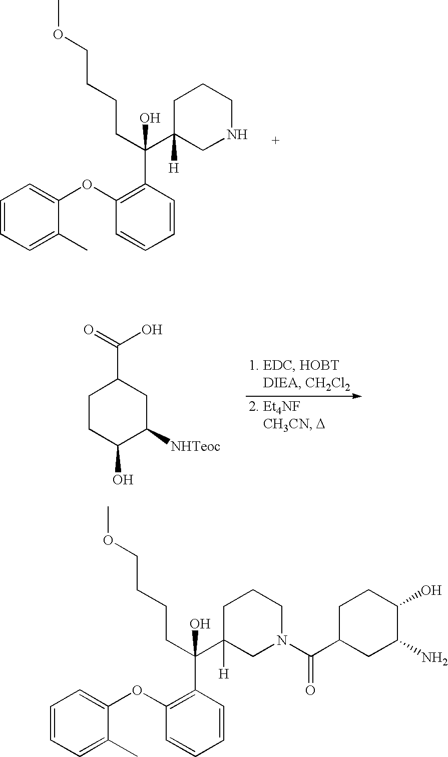

### Step 1. ((R)-3-((S)-1-(2-(o-tolyloxy)phenyl)-1-hydroxy-5-methoxypentyl)piperidin-1-yl)((3S*,4R*)-4-hydroxy-3-(2-(trimethylsilyl)ethoxycarbonylamino)cyclohexyl)-methanone

A mixture of (3R*,4S*)-4-hydroxy-3-(2-trimethylsilyl)ethoxycarbonylamino)cyclohexanecarboxylic acid (0.0380 g, 0.125 mmol, 1.0 equiv), (S)-1-(2-(o-tolyloxy)phenyl)-5-methoxy-1-((R)-piperidin-3-yl)pentan-1-ol (0.0157 g, 0.041 mmol, 0.32 equiv), EDC (0.150 g, 0.78 mmol, 6.2 equiv), HOBt (0.085 g, 0.63 mmol, 5.0 equiv), and DIEA (1.2 mL, 6.9 mmol, 55 equiv) in CH₂Cl₂ (2 mL) was stirred at rt for 48 h. After the solvents were removed, the residue was purified by reversed-phase HPLC (Phenomenex® Luna 5μ C18(2) 100 A, 250×21.20 mm, 5 micron, 70%→90% CH₃CN/H₂O, 0.1% CF₃COOH over 8 min and then 90% CH₃CN/H₂O, 0.1% CF₃COOH over 7 min, flow rate 25 mL/min) to give 0.0184 g (67%) of ((R)-3-((S)-1-(2-(o-tolyloxy)phenyl)-1-hydroxy-5-methoxypentyl)piperidin-1-yl)((3S*,4R*)-4-hydroxy-3-(2-(trimethylsilyl)ethoxycarbonylamino)cyclohexyl)methanone.

### Step 2. ((R)-3-((S)-1-(2-o-tolyloxy)phenyl)-1-hydroxy-5-methoxypentyl)piperidin-1-yl)((3S*,4R*)-3-amino-4-hydroxycyclohexyl)methanone

A mixture of ((R)-3-((S)-1-(2-(o-tolyloxy)phenyl)-1-hydroxy-5-methoxypentyl)piperidin-1-yl)((3S*,4R*)-4-hydroxy-3-(2-(trimethylsilyl)ethoxycarbonylamino)cyclohexyl)methanone (0.0184 g, 0.0275 mmol, 1.0 equiv), and Et₄NF (0.296 g, 1.98 mmol, 72 equiv) in CH₃CN (4 mL) was heated at 80° C. for 6 h. After the solvent was removed, the residue was purified by reversed-phase HPLC (Phenomenex® Luna 5μ C18(2) 100 A, 250×21.20 mm, 5 micron, 10%→90% CH₃CN/H₂O, 0.1% CF₃COOH over 13 min, flow rate 25 mL/min) to give the trifluoroacetate salt of ((R)-3-((S)-1-(2-(o-tolyloxy)phenyl)-1-hydroxy-5-methoxypentyl)piperidin-1-yl)((3S*,4R*)-3-amino-4-hydroxycyclohexyl)methanone (I-62B, 0.0138 g, 78%). LC-MS (3 min) t_{R}=1.44 min, m/z 525 (M+H⁺), 547 (M+Na⁺); ¹H NMR (400 MHz, CD₃OD) □ 7.55-7.52 (m, 1H), 7.17-6.87 (m, 5H), 6.62-6.42 (m, 2H), 4.31-3.58 (m, 3H), 3.30-2.76 (m, 6H), 2.48-0.77 (m, 23H).

### Example 7

The following compound was prepared following the procedures of Example 6:
I—((R)-3-((S)-1-(2-(o-tolyloxy)phenyl)-1-hydroxy-5-methoxypentyl)piperidin-1-yl)((3S,4R)-3-62A amino-4-hydroxycyclohexyl)methanone

### Example 8

### ((R)-3-((S)-1-(2-(o-tolyloxy)phenyl)-1-hydroxy-5-methoxypentyl)piperidin-1-yl)(2-aminopyridin-4-yl)methanone (I-22A)

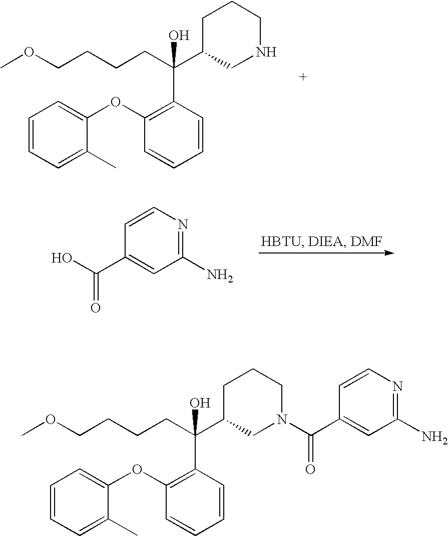

To a solution of (S)-1-(2-(o-tolyloxy)phenyl)-5-methoxy-1-((R)-piperidin-3-yl) pentan-1-ol (19.2 mg, 0.05 mmol) and 2-aminopyridine-4-carboxylic acid (7.0 mg, 0.05 mmol) in DMF (0.5 mL) was added DIEA (26 μL, 0.15 mmol), followed by HBTU (19.0 mg, 0.05 mmol). The resulting mixture was stirred at rt until no starting material remained (˜20 min). Preparative HPLC gave ((R)-3-((S)-1-(2-(o-tolyloxy)phenyl)-1-hydroxy-5-methoxypentyl)piperidin-1-yl)(2-aminopyridin-4-yl)methanone (I-35A, 24.0 mg, 95%) as its TFA salt. ¹H NMR (400 MHz, CD₃OD) □ 7.90, 7.80 (d, 1H), 7.66, 7.60 (d, 1H), 7.32 (m, 1H), 7.20-7.04 (m, 4H), 6.86-6.52 (m, 4H), 4.48 (d, 1H), 3.78, 3.46 (d, 1H), 3.24, 3.22 (s, 3H), 3.04-2.82 (m, 5H), 2.26 (s, 2H), 2.0-0.88 (m, 11H); LC-MS (3 min) m/z 504 (M+H⁺).

### Example 9

The following compounds of Formula I were prepared using the procedure in Example 8 from the piperidines and carboxylic acids: I-14A, I-15A, I-34A.

### Example 10

### ((R)-3-((S)-1-(2-(o-tolyloxy)phenyl)-1-hydroxy-5-methoxypentyl)piperidin-1-yl)((S)-3-aminopyrrolidin-1-yl)methanone (I-22A)

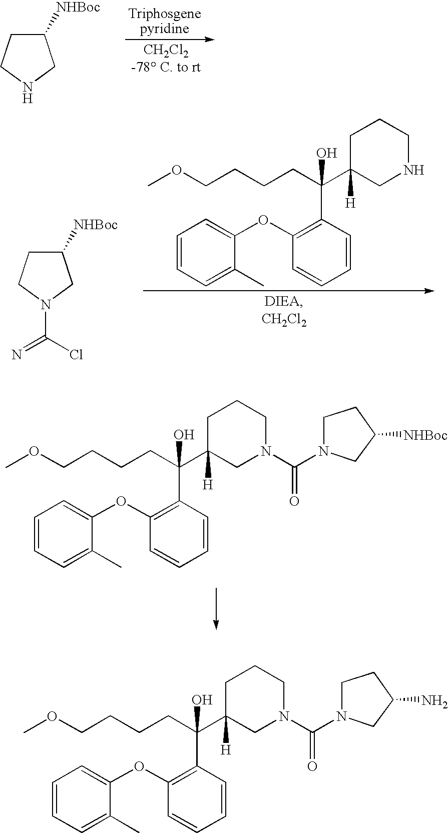

### Step 1. ((R)-3-((S)-1-(2-(o-tolyloxy)phenyl)-1-hydroxy-5-methoxypentyl)piperidin-1-yl)(tert-butyl (S)-3-aminopyrrolidin-1-ylcarbamate)methanone

A solution of tert-butyl (S)-pyrrolidin-3-ylcarbamate (186 mg, 1.0 mmol) in CH₂Cl₂ (5 mL) was cooled to −78° C. under N₂ and pyridine (0.12 mL, 1.5 mmol) was added, followed by a solution of triphosgene (234 mg, 0.79 mmol) in CH₂Cl₂ (3 mL). The mixture was stirred at −78° C. for 10 min and allowed to warm slowly to rt. After 30 min, an aliquot (1 mL, ˜0.12 mmol) of the reaction mixture was added to (S)-1-(2-(o-tolyloxy)phenyl)-5-methoxy-1-((R)-piperidin-3-yl)pentan-1-ol (20 mg, 0.05 mmol) and i-Pr₂NEt (0.20 mL, 1.1 mmol). The mixture was stirred at rt for 30 min. The mixture was concentrated and the residue was submitted directly to preparative HPLC to afford ((R)-3-((S)-1-(2-(o-tolyloxy)phenyl)-1-hydroxy-5-methoxypentyl)-piperidin-1-yl)(tert-butyl (S)-3-aminopyrrolidin-1-ylcarbamate)methanone (10 mg, 32%).

### Step 2. ((R)-3-((S)-1-(2-(o-tolyloxy)phenyl)-1-hydroxy-5-methoxypentyl)piperidin-1-yl)((S)-3-aminopyrrolidin-1-yl)methanone

((R)-3-((S)-1-(2-(o-tolyloxy)phenyl)-1-hydroxy-5-methoxypentyl)piperidin-1-yl)(tert-butyl (S)-3-aminopyrrolidin-1-ylcarbamate)methanone (10 mg, 0.17 mmol) was dissolved in 1:1 2N aq HCl/MeCN (20 mL). The mixture was left overnight at rt. LC/MS showed the reaction was complete. The mixture was neutralized with 5% aq NaOH solution and concentrated to remove the MeCN. The aq residue was extracted with CH₂Cl₂ (3×20 ml). The combined CH₂Cl₂ layers were dried over Na₂SO₄. After concentration, the residue was purified by preparative HPLC to afford ((R)-3-((S)-1-(2-(o-tolyloxy)phenyl)-1-hydroxy-5-methoxypentyl)piperidin-1-yl)((S)-3-aminopyrrolidin-1-yl)methanone (I-22A, 3.2 mg, 38%) as its TFA salt. ¹H NMR (400 MHz, CD₃OD) □ 7.64 (dd, 1H), 7.28 (d, 1H), 7.18-7.12 (m, 2H), 7.05 (t, 2H), 6.74 (d, 1H), 6.55 (d, 1H), 4.09 (d, 1H), 3.82 (m, 1H), 3.74-3.62 (m, 2H), 3.45 (m, 3H), 2.81 (t, 1H), 2.68 (t, 1H), 2.41 (m, 2H), 2.26 (m, 1H), 2.24 (s, 3H), 1.90 (m, 2H), 1.62 (d, 1H), 0.98 (m, 1H). LC-MS (3 min) m/Z 496 (M+H⁺).

### Example 11

The following compounds were prepared following the procedures described in Example 10, substituting the appropriate piperidines and carbamoyl chlorides 1-2A, I-6A, I-7A, I-8A, I-21A, I-22B, I-23A, I-24A, I-30A, I-31A, I-32A, I-38A, I-39A, I-40A, I-42A, I-45A, I-54A, I-70A, I-76A, I-77A, I-79A, I-80A, I-86A, I-87A, I-88A, I-91A, I-94A, I-95A, I-96A, I-108A, I-109A, I-110A, I-111A, I-112A, I-113A, I-114A, I-118A, I-118B^{a}, I-118C, I-119A, I-127A, I-128A, I-129A, I-129B, I-133A, I-134A, I-135A, I-136A, I-138A, I-139A, I-142A, I-143A, I-144A, I-145A, I-147A, I-148A, I-149A, I-151A, I-152A, I-156A, I-157A, I-160A, I-161A, I-162A, I-165A, I-165B^{a}, I-166A, I-168A, I-171A, I-172A, I-176A, I-187A, I-216A, ^{a} Minor isomer isolated by chromatography

### Example 12

### (S)-1-(2-o-tolyloxy)phenyl)-1-((R)-1-(1-(S)-3-aminopyrrolidin-1-yl)-2-nitrovinyl)piperidin-3-yl)-5-methoxypentan-1-ol (I-73B)

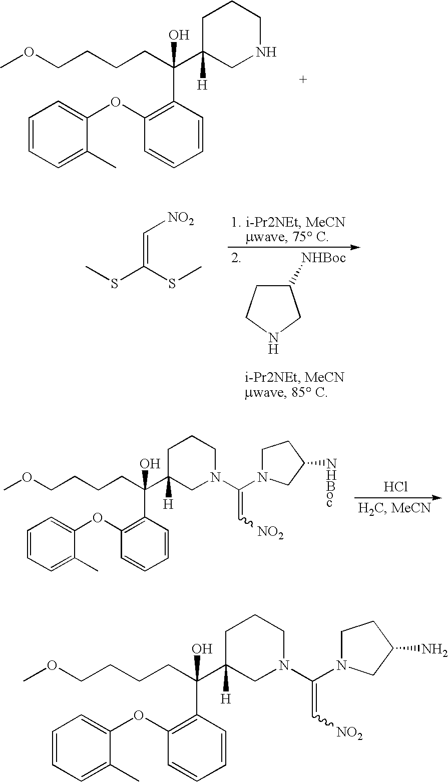

### Step 1. tert-butyl (S)-1-(1-((R)-3-((S)-1-(2-(o-tolyloxy)phenyl)-1-hydroxy-5-methoxypentyl)-piperidin-1-yl)-2-nitrovinyl)pyrrolidin-3-ylcarbamate

A solution of (S)-1-(2-o-tolyloxy)phenyl)-5-methoxy-1-((R)-piperidin-3-yl)pentan-1-ol (40 mg, 0.11 mmol), 1,1-bis(methylthio)-2-nitroethene (17 mg, 0.11 mmol), and DIEA (120 μL, 0.67 mmol) in MeCN (2 mL) was heated in a microwave oven at 75° C. for 40 min. LC-MS indicated the presence of (S)-1-(2-o-tolyloxy)phenyl)-5-methoxy-1-((R)-1-(1-(methylthio)-2-nitrovinyl)piperidin-3-yl)pentan-1-ol. tert-Butyl (S)-pyrrolidin-3-ylcarbamate (40 mg, 0.21 mmol) was added and the mixture was heated in a microwave oven at 85° C. for 35 min. The reaction mixture was submitted directly to preparative HPLC to afford tert-butyl (S)-1-(1-((R)-3-((S)-1-(2-(o-tolyloxy)phenyl)-1-hydroxy-5-methoxypentyl)piperidin-1-yl)-2-nitrovinyl)pyrrolidin-3-ylcarbamate (10.1 mg, 15%). LC-MS (3 min) m/z=639 (M+1).

### Step 2. (S)-1-(2-(o-tolyloxy)phenyl)-1-((R)-1-(1-((S)-3-aminopyrrolidin-1-yl)-2-nitrovinyl)-piperidin-3-yl)-5-methoxypentan-1-ol

tert-Butyl (S)-1-(1-((R)-3-((S)-1-(2-(o-tolyloxy)phenyl)-1-hydroxy-5-methoxypentyl)piperidin-1-yl)-2-nitrovinyl)pyrrolidin-3-ylcarbamate (9.4 mg, 0.015 mmol) was dissolved in a 1:1 mixture of 2N HCl solution/MeCN (20 mL). The mixture was left overnight at rt. The mixture was neutralized with 5% aq NaOH solution and concentrated to remove the MeCN. The residual aq mixture was extracted with CH₂Cl₂ (3×20 ml). The combined CH₂Cl₂ extracts were dried over Na₂SO₄. After concentration, the residue gave (S)-1-(2-o-tolyloxy)phenyl)-1-((R)-1-(1-((S)-3-aminopyrrolidin-1-yl)-2-nitrovinyl)piperidin-3-yl)-5-methoxypentan-1-ol (I-73B, 2.54 mg, 32%) as a HCl salt. ¹H NMR (400 MHz, CD₃OD) 7.66 (d, 1H), 7.30 (d, 1H), 7.20-7.14 (m, 2H), 7.12-7.04 (m, 2H), 6.76 (d, 1H), 6.53 (m, 1H), 4.28 (m, 1H), 4.07 (m, 2H), 3.23 (s, 3H), 3.22 (m, 1H), 2.46 (m, 1H), 2.26 (s, 3H), 2.24 (m, 1H), 0.98 (m, 1H), 0.89 (m, 1H).). LC-MS (3 min) m/z 539 (M+H⁺).

### Example 13

The following compounds were prepared using the procedures described in Example 12: I-57A, I-73A.

### Example 14

### 3-((S)-3-aminopiperidin-1-yl)-4-((R)-3-((S)-1-hydroxy-4-methoxy-1-(2-phenoxyphenyl)butyl)piperidin-1-yl)cyclobut-3-ene-1,2-dione (I-75A)

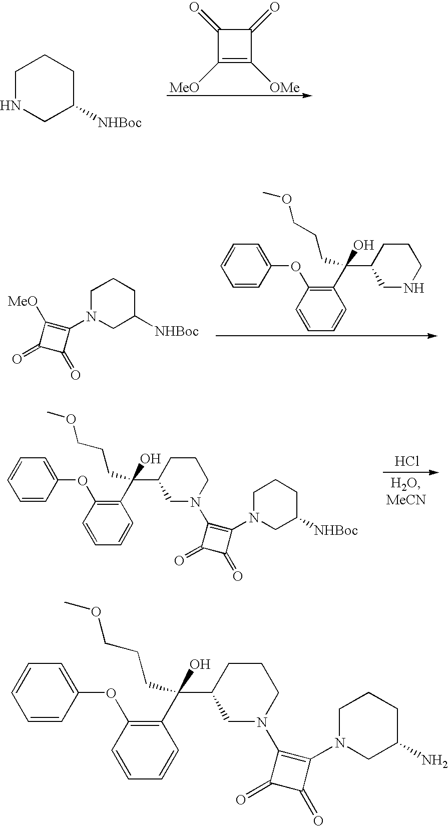

### Step 1. tert-butyl (S)-1-(2-methoxy-3,4-dioxocyclobut-1-enyl)piperidin-3-ylcarbamate

To a stirred suspension of tert-butyl (S)-piperidin-3-ylcarbamate (108 mg, 0.54 mmol) in MeCN (5 mL) was added solid 3,4-dimethoxycyclobut-3-ene-1,2-dione (77 mg, 0.54 mmol). The clear solution was stirred at rt for 3 d and evaporated to dryness. Flash chromatography on a 12-g silica cartridge eluted with a gradient from 0 to 100 and EtOAc in hexanes afforded tert-butyl (S)-1-(2-methoxy-3,4-dioxocyclobut-1-enyl)piperidin-3-ylcarbamate (130 mg, 78%). LC-MS (3 min) 1.25 min, m/z=311 (M+1).

### Step 2. tert-Butyl (S)-1-(2-((R)-3-((S)-1-hydroxy-4-methoxy-1-(2-phenoxyphenyl)butyl)piperidin-1-yl)-3,4-dioxocyclobut-1-enyl)piperidin-3-ylcarbamate

A solution of tert-butyl (S)-1-(2-methoxy-3,4-dioxocyclobut-1-enyl)piperidin-3-ylcarbamate (22 mg, 70 μmol), (S)-4-methoxy-1-(2-phenoxyphenyl)-1-((R)-piperidin-3-yl)butan-1-ol (26 mg, 70 μmol), and DIEA (50 mL, 0.28 mmol) in MeCN (1 mL) was stirred at rt for 18 h. A 10-mL Varian Chem-Elut cartridge was wetted with 5% aq HCl (5 mL) and allowed to stand for 5 min. The reaction mixture was applied and the cartridge was eluted with Et₂O (40 mL). The eluate was passed through a second 10-mL Chem-Elut cartridge that had been pre-wetted with satd aq NaHCO₃ (5 mL). Concentration of the eluate afforded a white solid (27 mg) which was purified by preparative reverse phase HPLC to afford tert-butyl (S)-1-(2-((R)-3-((S)-1-hydroxy-4-methoxy-1-(2-phenoxyphenyl)butyl)piperidin-1-yl)-3,4-dioxocyclobut-1-enyl)piperidin-3-ylcarbamate (16 mg, 35%). LC-MS (3 min) t_{R}=2.02 min, m/z=649 (M+1).

### Step 3. 3-((S)-3-aminopiperidin-1-yl)-4-((R)-3-((S)-1-hydroxy-4-methoxy-1-(2-phenoxyphenyl)-butyl)piperidin-1-yl)cyclobut-3-ene-1,2-dione

To a stirred solution of tert-butyl (S)-1-(2-((R)-3-((S)-1-hydroxy-4-methoxy-1-(2-phenoxyphenyl)butyl)piperidin-1-yl)-3,4-dioxocyclobut-1-enyl)piperidin-3-ylcarbamate (16 mg, 25 μmol) in MeCN (1 mL) was added 5% aq HCl (0.5 mL). The mixture was stirred for 52 h and basified by addition of solid K₂CO₃. The mixture was extracted with CH₂Cl₂ (100 mL). The organic layer was dried over Na₂SO₄ and concentrated to leave crude product (13 mg) which was purified by reverse phase preparative HPLC to afford 3-(S)-3-aminopiperidin-1-yl)-4-((R)-3-((S)-1-hydroxy-4-methoxy-1-(2-phenoxyphenyl)butyl)piperidin-1-yl)cyclobut-3-ene-1,2-dione as the trifluoroacetate salt (I-75A, 6.5 mg, 39%). ¹H NMR (MeOH-d₄) δ 0.90 (m, 1H), 1.2-1.9 (14H), 2.12 (m, 1H), 2.36 (m, 2H), 3.04 (m, 1H), 3.22 (s, 3H), 3.27 (m, 2H), 3.41 (m, 1H), 3.50 (m, 1H), 3.60 (m, 1H), 3.98 (m, 1H), 4.19 (m, 1H), 4.43 (m, 1H), 6.83 (d, 1H), 6.93 (d, 2H), 7.07 (t, 1H), 7.17 (m, 1H), 7.22 (m, 1H), 7.32 (m, 2H), 7.66 (d, 1H); LC-MS (16 min) t_{R}=6.23 min, m/z=548 (M+1), 530 (M−17).

### Example 15

The following compound was prepared following the procedures described in Example 14: I-72A.

### Example 16

### ((R)-3-((S)-1-(2-(o-Tolyloxy)phenyl)-1-hydroxy-5-methoxypentyl)piperidin-1-yl)((1S,3R,4R)-3-hydroxy-4-(methylamino)cyclopentyl)methanone (I-58A)

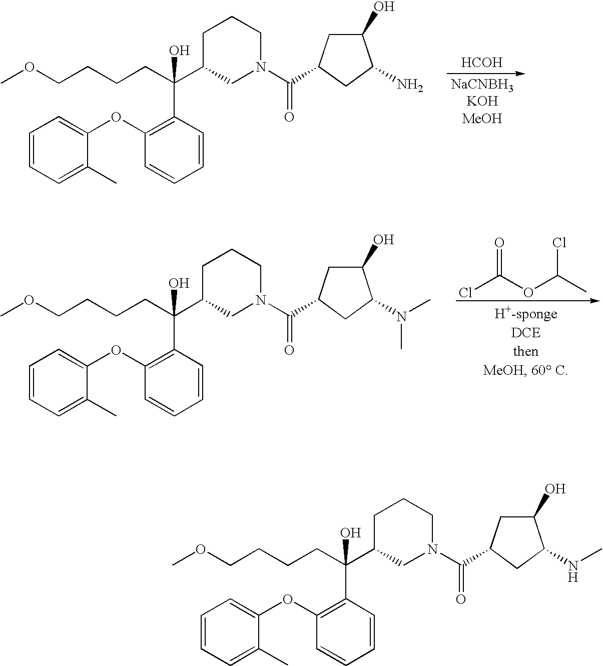

### Step 1. ((R)-3-((S)-1-(2-(o-Tolyloxy)phenyl)-1-hydroxy-5-methoxypentyl)piperidin-1-yl)((1S,3R,4R)-3-(dimethylamino)-4-hydroxycyclopentyl)methanone

To a solution of ((R)-3-((S)-1-(2-(o-tolyloxy)phenyl)-1-hydroxy-5-methoxypentyl)piperidin-1-yl)((1S,3R,4R)-3-amino-4-hydroxycyclopentyl)methanone (16.8 mg, 0.033 mmol) in MeOH (0.2 mL) were added formaldehyde (37 wt % in water, 2.7 mg, 0.033 mmol) and solid KOH (0.7 mg), followed by NaCNBH₃ (6.5 mg, 0.099 mmol). The resulting mixture was stirred at rt until no starting material remained (˜1 h). Preparative HPLC gave ((R)-3-(S)-1-(2-o-tolyloxy)phenyl)-1-hydroxy-5-methoxypentyl)piperidin-1-yl)((1S,3R,4R)-3-(dimethylamino)-4-hydroxycyclopentyl)methanone (9.1 mg, 51%). ¹H NMR (400 MHz, CD₃OD) δ 7.64 (d, 1H), 7.26 (m, 1H), 7.14 (m, 2H), 7.04 (m, 2H), 6.72 (d, 1H), 6.58 (d, 1H), 4.86, 4.44 (two d, 1H), 4.34 (m, 1H), 4.24, 3.94 (two d, 1H), 3.40 (m, 2H), 3.26 (t, 2H), 3.24 (s, 3H), 3.18 (dd, 1H), 2.98 (s, 3H), 2.90 (s, 3H), 2.64 (dd, 1H), 2.42 (m, 1H), 2.32 (m, 2H), 2.24, 2.22 (two s, 3H), 2.04 (m, 1H), 1.98-0.84 (m, 11); LC-MS (3 min) m/z 539 (M+H⁺).

### Step 2. ((R)-3-((S)-1-(2-(o-Tolyloxy)phenyl)-1-hydroxy-5-methoxypentyl)piperidin-1-yl)((1S,3R,4R)-3-hydroxy-4-(methylamino)cyclopentyl)methanone

To a solution of ((R)-3-((S)-1-(2-(o-tolyloxy)phenyl)-1-hydroxy-5-methoxypentyl)piperidin-1-yl)((1S,3R,4R)-3-(dimethylamino)-4-hydroxycyclopentyl)methanone (5.9 mg, 0.011 mmol) and 1,8-bis(dimethylamino)naphthalene (Proton-sponge®, 6.9 mg, 0.032 mmol) in 1,2-dichloroethane (0.5 mL) at rt was added 1-chloroethyl chloroformate (2.4 mg, 0.016 mmol). The resulting solution was stirred at rt until no starting material remained by LC-MS. 1,2-Dichloroethane was removed in vacuo, and the residue was redissolved in MeOH (0.5 mL), and heated at 60° C. for 20 min. Preparative HPLC gave ((R)-3-(S)-1-(2-o-tolyloxy)phenyl)-1-hydroxy-5-methoxypentyl)-piperidin-1-yl)((1S,3R,4R)-3-hydroxy-4-(methylamino)cyclopentyl)methanone (I-58A, 2.4 mg, 42%) as its TFA salt. ¹H NMR (400 MHz, CD₃OD) δ 7.64 (d, 1H), 7.26 (m, 1H), 7.16 (m, 2H), 7.04 (m, 2H), 6.72 (d, 1H), 6.58, 6.56 (two d, 1H), 4.86, 4.44 (two d, 1H), 4.24, 4.16 (m, 1H), 4.24, 3.92 (two d, 1H), 3.56, 3.44 (m, 2H), 3.24 (s, 3H), 3.22 (t, 2H), 3.18 (dd, 1H), 2.98 (m, 1H), 2.74 (s, 3H), 2.62 (dd, 1H), 2.52-2.24 (m, 2H), 2.24, 2.22 (two s, 3H), 2.04-0.84 (m, 12). LC-MS (3 min) m/z 525 (M+H⁺).

### Example 17

The following analogs were prepared using the procedures described in Example 16: I-58B, I-58C.

### Example 18

### ((3R,4S)-3-amino-4-hydroxypyrrolidin-1-yl)((S)-2-(S)-1-(6-fluoro-3′-methylbiphenyl-2-yl)-5-methoxypentyl)morpholino)methanone (I-107A)

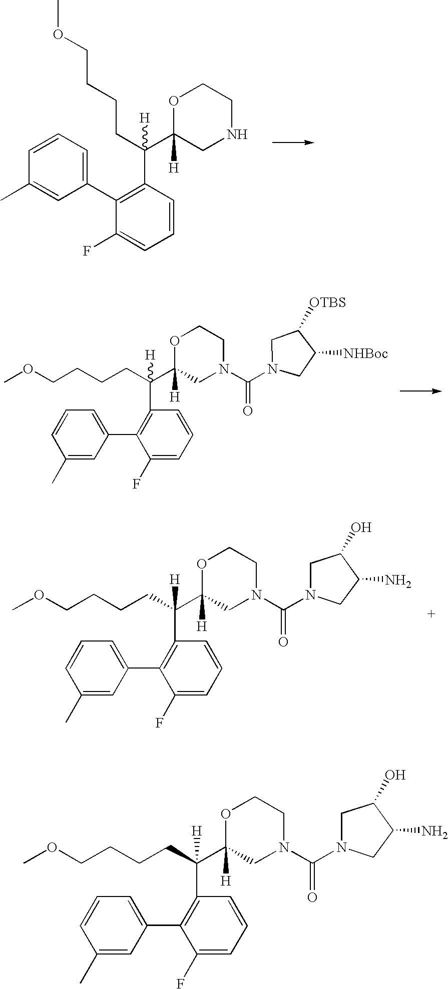

### Step 1. tert-butyl (3R,4S)-4-(tert-butyldimethylsilyloxy)-1-((2S)-2-(1-(6-fluoro-3′-methylbiphenyl-2-yl)-5-methoxypentyl)morpholine-4-carbonyl)pyrrolidin-3-ylcarbamate

A small vial was charged with triphosgene (12.5 mg, 0.042 mmol) and anhydrous CH₂Cl₂ (0.5 mL) and the solution was chilled to −78° C. A solution of the HCl salt of (2S)-2-(1-(6-fluoro-3′-methylbiphenyl-2-yl)-5-methoxypentyl)morpholine (17.20 mg, 0.042 mmol) and pyridine (7 μL, 2 eq) in anhydrous CH₂Cl₂ (0.5 mL) was added dropwise within 10 min. After the addition, the reaction mixture was allowed to warm to rt and stirred for 1 h. A solution of tert-butyl (3R,4S)-4-(tert-butyldimethylsilyloxy)pyrrolidin-3-ylcarbamate (51 mg, 0.126 mmol) and triethylamine (11 μL) in anhydrous CH₂Cl₂ (1 mL) was added in one portion (the color turned to light yellow at once) and the mixture was stirred for 30 min. The organic solvent was removed under reduced pressure and purified by preparative HPLC to afford tert-butyl (3R,4S)-4-tert-butyldimethylsilyloxy)-1-((2S)-2-(1-(6-fluoro-3′-methylbiphenyl-2-yl)-5-methoxypentyl)morpholine-4-carbonyl)pyrrolidin-3-ylcarbamate (19 mg, yield: 63%). MS m/z 714 (M+H)⁺.

### Step 2. ((3R,4S)-3-amino-4-hydroxypyrrolidin-1-yl)((S)-2-((S)-1-(6-fluoro-3′-methylbiphenyl-2-yl)-5-methoxypentyl)morpholino)methanone

tert-butyl (3R,4S)-4-(tert-butyldimethylsilyloxy)-1-((2S)-2-(1-(6-fluoro-3′-methylbiphenyl-2-yl)-5-methoxypentyl)morpholine-4-carbonyl)pyrrolidin-3-ylcarbamate (19 mg, 0.027 mmol) was dissolved in 1 N HCl in MeOH and stirred at 50° C. for 10 min. The solvent was evaporated and the residue was purified by preparative HPLC to give the title compound ((3R,4S)-3-amino-4-hydroxypyrrolidin-1-yl)((S)-2-((S)-1-(6-fluoro-3′-methylbiphenyl-2-yl)-5-methoxypentyl)morpholino)methanone as its TFA salt (5.54 mg, yield 35%) and ((3R,4S)-3-amino-4-hydroxypyrrolidin-1-yl)((S)-2-((R)-1-(6-fluoro-3′-methylbiphenyl-2-yl)-5-methoxypentyl)morpholino)methanone as its TFA salt (6.03 mg, yield 38%). MS m/z 500 (M+H)⁺.

### Example 19

### ((3R,4S)-3-amino-4-hydroxypyrrolidin-1-yl)(3-(1-(6-fluoro-3′-methylbiphenyl-2-yl)-5-methoxypentyl)phenyl)methanone

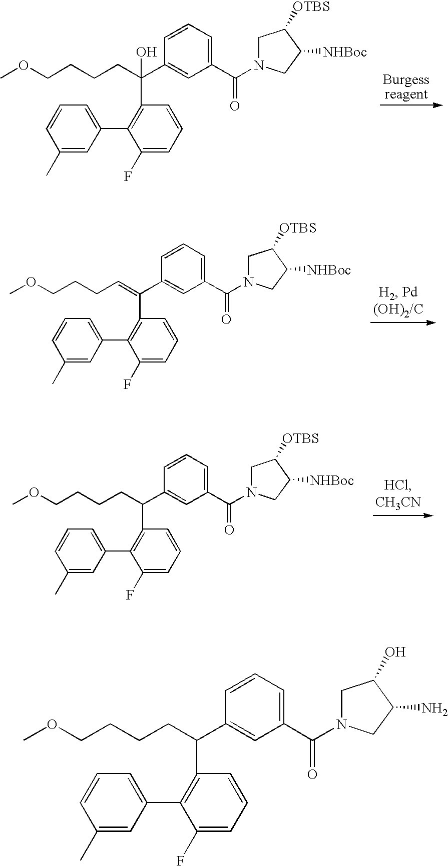

### Step 1. tert-butyl (3R,4S)-4-tert-butyldimethylsilyloxy)-1-(3-((Z)-1-(6-fluoro-3′-methylbiphenyl-2-yl)-5-methoxypent-1-enyl)benzoyl)pyrrolidin-3-ylcarbamate

To a stirred solution of tert-butyl (3R,4S)-4-(tert-butyldimethylsilyloxy)-1-(3-(1-(6-fluoro-3′-methylbiphenyl-2-yl)-1-hydroxy-5-methoxypentyl)benzoyl)pyrrolidin-3-ylcarbamate (50 mg, 69.4 μmol) in toluene (10 mL) was added Burgess reagent (33.2 mg, 138.8 μmol). The reaction mixture was heated under reflux overnight. The mixture was cooled to rt and concentrated in vacuo. The residue was purified by preparative tlc (1:1 petroleum ether/EtOAc) to give tert-butyl (3R,4S)-4-(tert-butyldimethylsilyloxy)-1-(3-((Z)-1-(6-fluoro-3′-methylbiphenyl-2-yl)-5-methoxypent-1-enyl)benzoyl)pyrrolidin-3-ylcarbamate (20 mg, 41%). ¹H NMR (CDCl₃, 400 MHz): δ 0.09 (m, 6H), 0.82-0.94 (m, 9H), 1.45 (s, 9H), 1.62 (m, 2H), 2.16 (m, 5H), 3.24 (m, 3H), 3.36-3.78 (m, 6H), 4.12 (m, 2H), 4.32 (m, 1H), 4.58 (m, 2H), 6.02 (m, 1H), 6.76 (m, 2H), 6.96-7.18 (m, 9H).

### Step 2. tert-butyl (3R,4S)-4-(tert-butyldimethylsilyloxy)-1-(3-(1-(6-fluoro-3′-methylbiphenyl-2-yl)-5-methoxypentyl)benzoyl)pyrrolidin-3-ylcarbamate

To a solution of tert-butyl (3R,4S)-4-(tert-butyldimethylsilyloxy)-1-(3-((Z)-1-(6-fluoro-3′-methylbiphenyl-2-yl)-5-methoxypent-1-enyl)benzoyl)pyrrolidin-3-ylcarbamate (20 mg, 28 μmol) in dry methanol under a hydrogen gas atmosphere was added Pd(OH)₂/C as the catalyst. The reaction mixture was stirred at rt for 3 h, filtered and concentrated to give tert-butyl (3R,4S)-4-tert-butyldimethylsilyloxy)-1-(3-(1-(6-fluoro-3′-methylbiphenyl-2-yl)-5-methoxypentyl)benzoyl)pyrrolidin-3-ylcarbamate (19 mg, 96.4%). MS (E/Z): 705 (M+H⁺)

### Step 3. ((3R,4S)-3-amino-4-hydroxypyrrolidin-1-yl)(3-(1-(6-fluoro-3′-methylbiphenyl-2-yl)-5-methoxypentyl)phenyl)methanone

tert-butyl (3R,4S)-4-(tert-butyldimethylsilyloxy)-1-(3-(1-(6-fluoro-3′-methylbiphenyl-2-yl)-5-methoxypentyl)benzoyl)pyrrolidin-3-ylcarbamate (23 mg, 32 μmol) was dissolved in 2 M HCl in MeCN (10 mL). The reaction mixture was stirred at 60° C. for 4 h. The solution was neutralized by addition of satd aq NaHCO₃ and extracted with CH₂Cl₂ (3×15 mL). The combined organic extracts were dried over Na₂SO₄. The solvent was removed and the residue was purified by preparative HPLC to give ((3R,4S)-3-amino-4-hydroxypyrrolidin-1-yl)(3-(1-(6-fluoro-3′-methylbiphenyl-2-yl)-5-methoxypentyl)phenyl)methanone (0.6 mg, 3.8%). ¹H NMR (CDCl₃, 400 MHz): δ=0.87 (m, 1H), 1.10-1.40 (m, 11H), 1.48 (m, 1H), 1.60 (m, 2H), 2.02 (m, 2H), 2.26-2.43 (m, 3H), 3.24 (s, 3H), 3.50-3.30 (m, 6H), 3.92 (m, 2H), 4.18 (m, 2H), 6.58-6.74 (m, 2H), 6.96-7.39 (m, 9H). MS: 491.3 (M+H⁺).

### Example 20

### 6-((S)-1-((R)-1-((1S,3R,4S)-3-Amino-4-hydroxycyclopentanecarbonyl)piperidin-3-yl)-1-hydroxy-5-methoxypentyl)biphenyl-3-carbonitrile (I-188A)

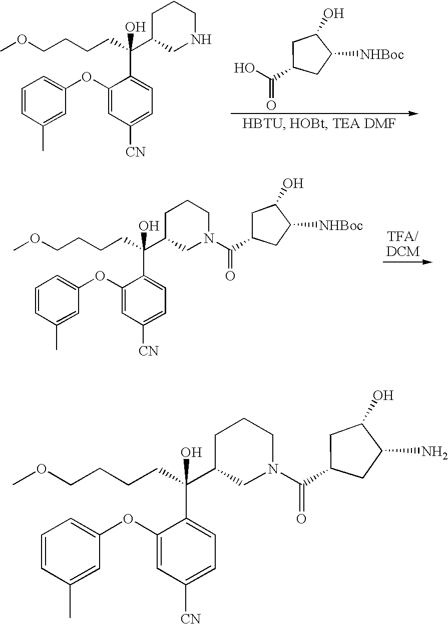

### Step 1. tert-Butyl (1R,2S,4S)-4-((R)-3-((S)-1-(5-cyano-3′-methylbiphenyl-2-yl)-1-hydroxy-5-methoxypentyl)piperidine-1-carbonyl)-2-hydroxycyclopentylcarbamate

To a solution of 6-((S)-1-hydroxy-5-methoxy-1-((R)-piperidin-3-yl)pentyl)-3′-methylbiphenyl-3-carbonitrile TFA salt (10.1 mg, 0.021 mmol), Et₃N (11 μL) and (1S,3R,4S)-3-(tert-butoxycarbonylamino)-4-hydroxycyclopentanecarboxylic acid (5.5 mg, 0.024 mmol) in DMF (2 mL) was added HBTU (9.0 mg), followed by HOBt (3.2 mg) and the resulting mixture was stirred at rt for 1 h. The reaction mixture was purified by preparative HPLC to give tert-butyl (1R,2S,4S)-4-((R)-3-((S)-1-(5-cyano-3′-methylbiphenyl-2-yl)-1-hydroxy-5-methoxypentyl)piperidine-1-carbonyl)-2-hydroxycyclopentylcarbamate (10.0 mg, 81%). MS m/z 620 (M+H⁺).

### Step 2. 6-((S)-1-((R)-1-((1S,3R,4S)-3-Amino-4-hydroxycyclopentanecarbonyl)piperidin-3-yl)-1-hydroxy-5-methoxypentyl)-3′-methylbiphenyl-3-carbonitrile

tert-Butyl (1R,2S,4S)-4-((R)-3-((S)-1-(5-cyano-3′-methylbiphenyl-2-yl)-1-hydroxy-5-methoxypentyl)piperidine-1-carbonyl)-2-hydroxycyclopentylcarbamate (10.0 mg, 0.16 mmol) was dissolved in 1:4 TFA/DCM v/v (5 mL). The solution was stirred for 30 min and evaporated. The residue was purified by preparative HPLC to give 6-((S)-1-((R)-1-(1S,3R,4S)-3-amino-4-hydroxycyclopentanecarbonyl)piperidin-3-yl)-1-hydroxy-5-methoxypentyl)-3′-methylbiphenyl-3-carbonitrile as a TFA salt (5.7 mg, 56%). MS m/z 520 (M+H⁺). ¹H NMR (400 MHz, CD₃OD) δ (ppm) 7.99 (d, J=0.84 Hz, 1H), 7.70 (t, J=7.4 Hz, 1H), 7.33-7.22 (m, 3H), 7.06-6.95 (m, 2H), 4.57 and 4.42 (m, 1H), 4.31 and 4.24 (m, 1H), 3.93 (m, 1H), 4.50 (m, 1H), 3.35 and 3.34 (s, 3H), 3.30 and 3.16 (m, 1H), 3.28 (m 2H), 3.04 and 2.90 (m, 1H), 2.55-1.18 (m, 18H), 0.85 (m 1H)

### Example 21

### N—((S)-4-(6-chloro-3′-ethylbiphenyl-2-yl)-4-hydroxy-4-((R)-1-(4-((methylamino)methyl)benzoyl)piperidin-3-yl)butyl)acetamide (I-314A)

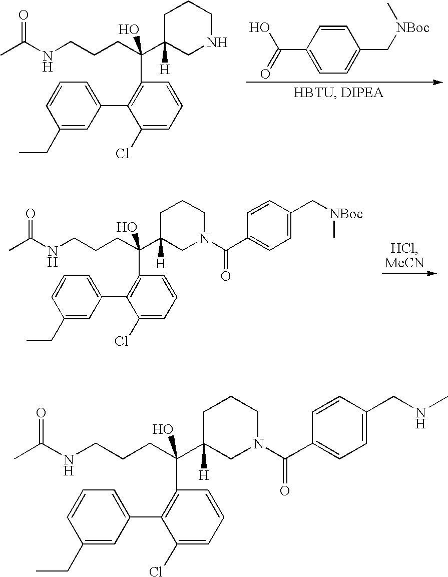

### Step 1. tert-butyl 4-((R)-3-(S)-4-acetamido-1-(6-chloro-3′-ethylbiphenyl-2-yl)-1-hydroxybutyl)piperidine-1-carbonyl)benzyl(methyl)carbamate

A solution of N—((S)-4-(6-chloro-3′-ethylbiphenyl-2-yl)-4-hydroxy-4-((R)-piperidin-3-yl)butyl)acetamide (48 mg, 0.10 mmol) in 1 mL of DMF at 25° C. was treated with 4-((tert-butoxycarbonyl(methyl)amino)methyl)benzoic acid (33 mg, 0.12 mmol), i-Pr₂NEt (0.089 mL, 0.5 mmol), and HBTU (47 mg, 0.12 mmol). After 24 h, H₂O was added and the mixture was extracted with EtOAc. The organic extracts were washed (1N aq HCl, 1N aq NaOH, H₂O, brine), dried (Na₂SO₄), concentrated under reduced pressure, and subjected to flash chromatography to provide tert-butyl 4-((R)-3-((S)-4-acetamido-1-(6-chloro-3′-ethylbiphenyl-2-yl)-1-hydroxybutyl)piperidine-1-carbonyl)benzyl(methyl)carbamate as a colorless oil (50 mg, 71%). MS (m/z) 676.3 (M+H⁺).

### Step 2. N—((S)-4-(6-chloro-3′-ethylbiphenyl-2-yl)-4-hydroxy-4-((R)-1-(4-((methylamino)methyl)benzoyl)piperidin-3-yl)butyl)acetamide

A solution of tert-butyl 4-((R)-3-((S)-4-acetamido-1-(6-chloro-3′-ethylbiphenyl-2-yl)-1-hydroxybutyl)piperidine-1-carbonyl)benzyl(methyl)carbamate (50 mg, 0.074 mmol) in 3 mL of CH₃CN at 25° C. was treated with 3 mL of aqueous 2N HCl. After 24 h, the mixture was concentrated under reduced pressure to provide N-[(4S)-4-(6-chloro-3′-ethyl-2-biphenylyl)-4-hydroxy-4-[(3R)-1-([4-[(methylamino)methyl]phenyl]carbonyl)-3-piperidinyl]butyl]acetamide as a white solid (39 mg, quantitative). MS (m/z) 576.2 (M+H⁺).

### Example 22

The following compounds were prepared following procedures analogous to those described in Example 21: I-239A, I-241A, I-243A, I-258A, I-258B, I-260A, I-263A, I-264A, I-267A, I-269A, I-271A, I-272A, I-274A, I-276A, I-277A, I-282A, I-286A, I-288A, I-288B, I-289A, I-290A, I-291A, I-293A, I-300A, I-301A, I-302A, I-303A, I-303B, I-304A, I-306A, I-307B, I-308A, I-309A, I-310A, I-311A, I-314A, I-315A, I-317A, I-318A, I-319A, I-324A, I-329A, I-338, I-339, I-340.

### Example 23

### Methyl (S)-4-(6-chloro-3′-ethylbiphenyl-2-yl)-4-hydroxy-4-((R)-1-(4-((methylamino)methyl)benzoyl)piperidin-3-yl)butylcarbamate (I-307A)

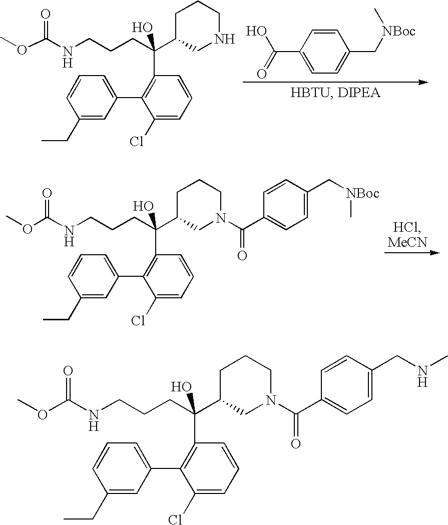

### Step 1. methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate

A solution of methyl (S)-4-(6-chloro-3′-ethylbiphenyl-2-yl)-4-hydroxy-4-((R)-piperidin-3-yl)butylcarbamate (30 mg, 0.07 mmol) in 1 mL of DMF at 25° C. was treated with 4-((tert-butoxycarbonyl(methyl)amino)methyl)benzoic acid (21 mg, 0.08 mmol), i-Pr₂NEt (0.063 mL, 0.37 mmol), and HBTU (30 mg, 0.08 mmol). After 1 h, H₂O was added and the mixture was extracted with EtOAc. The organic extracts were washed (1N HCl, 1N NaOH, H₂O, brine), dried (Na₂SO₄), concentrated under reduced pressure, and subjected to flash chromatography to provide methyl (S)-4-(6-chloro-3′-ethylbiphenyl-2-yl)-4-hydroxy-4-((R)-1-(4-((N-t-butoxycarbonyl-N-methyl)amino)methyl)benzoyl)piperidin-3-yl)butylcarbamate as a colorless oil (24 mg, 51%). MS (m/z) 692.3 (M+H⁺).

### Step 2. methyl {(4S)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate

A solution of methyl (S)-4-(6-chloro-3′-ethylbiphenyl-2-yl)-4-hydroxy-4-((R)-1-(4-(((N-t-butoxycarbonyl-N-methyl)amino)methyl)benzoyl)piperidin-3-yl)butylcarbamate (24 mg, 0.034 mmol) in 3 mL of CH₃CN at 25° C. was treated with 3 mL of aqueous 2N HCl. After 24 h, the mixture was concentrated under reduced pressure to provide methyl {(4S)-4-(6-chloro-3′-ethyl-2-biphenylyl)-4-hydroxy-4-[(3R)-1-({4-[(methylamino)methyl]phenyl}carbonyl)-3-piperidinyl]butyl}carbamate as a white solid (17 mg, 81%). MS (m/z) 592.2 (M+H⁺).

### Example 24

The following piperidines were prepared following procedures analogous to those described in Example 23 using the appropriate amine intermediate and the indicated acid in place of 4-{[{[(1,1-dimethylethyl)oxy]carbonyl}(methyl)amino]methyl}benzoic acid in Step 1:

| | Product | Acid used in Step 1 |
|---|---|---|
| I-245A | methyl 4-((R)-1-((1R,2S)-2- | (1R,2S)-2-({[(1,1- |
| | aminocyclopentanecarbonyl)piperidin-3- | dimethylethyl)oxy]carbonyl}amino)cyclopentanecarboxylic |
| | yl)-4-(3′-ethyl-6-fluorobiphenyl-2-yl)-4- | acid |
| | hydroxybutylcarbamate | |
| I-247A | (trans-4-aminocyclohexyl)((3R)-3-(1-(6- | trans-4-({[(1,1- |
| | chloro-3′-ethylbiphenyl-2-yl)-1-hydroxy- | dimethylethyl)oxy]carbonyl}amino)cyclohexanecarboxylic |
| | 5-methoxypentyl)piperidin-1- | acid |
| | yl)methanone | |
| I-248A | methyl (R)-4-(3′-ethyl-6-fluorobiphenyl- | (4R)-1-{[(1,1- |
| | 2-yl)-4-hydroxy-4-((R)-1-((2S,4R)-4- | dimethylethyl)oxy]carbonyl}-4-hydroxy- |
| | hydroxypyrrolidine-2-carbonyl)piperidin- | L-proline |
| | 3-yl)butylcarbamate | |
| I-262A | (4-(aminomethyl)phenyl)((3R)-3-(1-(6- | 4-[({[(1,1- |
| | chloro-3′-ethylbiphenyl-2-yl)-1-hydroxy- | dimethylethyl)oxy]carbonyl}amino)methyl]benzoic |
| | 5-methoxypentyl)piperidin-1- | acid |
| | yl)methanone | |
| I-266A | (6-(aminomethyl)pyridin-3-yl)((3R)-3-(1- | 6-[({[(1,1- |
| | (6-chloro-3′-ethylbiphenyl-2-yl)-1- | dimethylethyl)oxy]carbonyl}amino)methyl]- |
| | hydroxy-5-methoxypentyl)piperidin-1- | 3-pyridinecarboxylic acid |
| | yl)methanone | |
| I-268A | (3-(aminomethyl)phenyl)((R)-2-((R)-1- | 3-[({[(1,1- |
| | (6-chloro-3′-ethylbiphenyl-2-yl)-1- | dimethylethyl)oxy]carbonyl}amino)methyl]benzoic |
| | hydroxy-5- | acid |
| | methoxypentyl)morpholino)methanone | |
| I-285A | (4-(aminomethyl)cyclohexyl)((2R)-2- | 4-[({[(1,1- |
| | ((1R)-1-(6-chloro-2′-fluoro-5′- | dimethylethyl)oxy]carbonyl}amino)methyl]cyclohexanecarboxylic |
| | methylbiphenyl-2-yl)-1-hydroxy-5- | acid |
| | methoxypentyl)morpholino)methanone | |
| I-287A | (4-(2-aminoethyl)phenyl)((3R)-3-(1-(6- | 4-[2-({[(1,1- |
| | chloro-3′-ethylbiphenyl-2-yl)-1-hydroxy- | dimethylethyl)oxy]carbonyl}amino)ethyl]benzoic |
| | 5-methoxypentyl)piperidin-1- | acid |
| | yl)methanone | |
| I-297A | methyl 4-(6-chloro-3′-ethylbiphenyl-2- | (1R,3S)-3-[{[(1,1- |
| | yl)-4-hydroxy-4-((R)-1-((1R,3S)-3- | dimethylethyl)oxy]carbonyl}(methyl)amino]cyclopentanecarboxylic |
| | (methylamino)cyclopentanecarbonyl)piperidin- | acid |
| | 3-yl)butylcarbamate | |
| I-312A | methyl 4-((R)-1-(trans-4- | trans-4-[({[(1,1- |
| | (aminomethyl)cyclohexanecarbonyl)piperidin- | dimethylethyl)oxy]carbonyl}amino)methyl]cyclohexanecarboxylic |
| | 3-yl)-4-(6-chloro-3′-ethylbiphenyl- | acid |
| | 2-yl)-4-hydroxybutylcarbamate | |
| I-316A | methyl 4-(6-chloro-3′-ethylbiphenyl-2- | 4-{[{[(1,1- |
| | yl)-4-hydroxy-4-((R)-1-(4- | dimethylethyl)oxy]carbonyl}(methyl)amino]methyl}benzoic |
| | ((methylamino)methyl)benzoyl)piperidin- | acid |
| | 3-yl)butylcarbamate | |
| I-322A | methyl 4-((R)-1-(4-(aminomethyl)-2- | 4-[({[(1,1- |
| | fluorobenzoyl)piperidin-3-yl)-4-(6- | dimethylethyl)oxy]carbonyl}amino)methyl]- |
| | chloro-3′-ethylbiphenyl-2-yl)-4- | 2-fluorobenzoic acid |
| | hydroxybutylcarbamate | |
| I-325A | methyl 4-(6-chloro-3′-ethylbiphenyl-2- | 4-{[{[(1,1- |
| | yl)-4-((R)-1-(4- | dimethylethyl)oxy]carbonyl}(ethyl)amino]methyl}benzoic |
| | ((ethylamino)methyl)benzoyl)piperidin- | acid |
| | 3-yl)-4-hydroxybutylcarbamate | |
| I-328A | methyl 4-(6-chloro-3′-ethylbiphenyl-2- | 4-{[{[(1,1-dimethylethyl)oxy]carbonyl}(1- |
| | yl)-4-hydroxy-4-((R)-1-(4- | methylethyl)amino]methyl}benzoic acid |
| | ((isopropylamino)methyl)benzoyl)piperidin- | |
| | 3-yl)butylcarbamate | |
| I-337A | methyl {4-(6-chloro-3′-ethyl-2- | trans-4-{[{[(1,1- |
| | biphenylyl)-4-hydroxy-4-[(3R)-1-({trans- | dimethylethyl)oxy]carbonyl}(methyl)amino]methyl}cyclohexanecarboxylic |
| | 4- | acid |
| | [(methylamino)methyl]cyclohexyl}carbonyl)- | |
| | 3-piperidinyl]butyl}carbamate | |

### Example 25

### ((1S,3R,4S)-3-amino-4-hydroxycyclopentyl)((R)-2-((R)-1-(6-chloro-3′-ethylbiphenyl-2-yl)-1-hydroxypent-4-enyl)morpholino)methanone (I-222A)

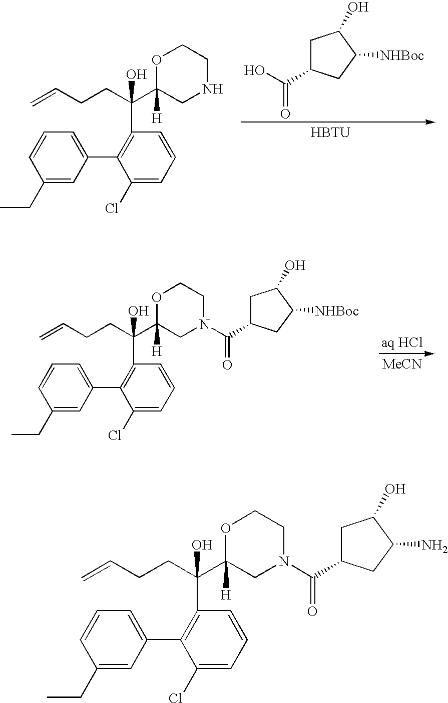

### Step 1. tert-butyl (1R,2S,4S)-4-((R)-2-((R)-1-(6-chloro-3′-ethylbiphenyl-2-yl)-1-hydroxypent-4-enyl)morpholine-4-carbonyl)-2-hydroxycyclopentylcarbamate

To a solution of (R)-1-(6-chloro-3′-ethylbiphenyl-2-yl)-1-((R)-morpholin-2-yl)pent-4-en-1-ol (55 mg, 0.14 mmol), (1S,3R,4S)-3-(tert-butoxycarbonylamino)-4-hydroxycyclopentanecarboxylic acid (35 mg, 0.14 mmol), and i-Pr₂NEt (54 mg, 0.42 mmol) in 2 mL of DMF was added HBTU (64 mg, 0.17 mmol). The reaction was stirred for 2 h and diluted with 10 mL water. It was extracted with EtOAc (3×10 mL). The combined organic extracts were dried over Na₂SO₄ and filtered, followed by concentration under reduced pressure. This afforded tert-butyl (1R,2S,4S)-4-((R)-2-((R)-1-(6-chloro-3′-ethylbiphenyl-2-yl)-1-hydroxypent-4-enyl)morpholine-4-carbonyl)-2-hydroxycyclopentylcarbamate which was used without purification.

### Step 2. ((1S,3R,4S)-3-amino-4-hydroxycyclopentyl)((R)-2-((R)-1-(6-chloro-3′-ethylbiphenyl-2-yl)-1-hydroxypent-4-enyl)morpholino)methanone

To a solution of tert-butyl (1R,2S,4S)-4-((R)-2-((R)-1-(6-chloro-3′-ethylbiphenyl-2-yl)-1-hydroxypent-4-enyl)morpholine-4-carbonyl)-2-hydroxycyclopentylcarbamate (85 mg, 0.14 mmol) in 10 mL of MeCN was added 10 mL of 2N aq HCl. The reaction was stirred overnight. It was basified with 10N aq NaOH to pH=14 and extracted with CH₂Cl₂ (3×10 ml). The combined organic extracts were dried over Na₂SO₄ and filtered, followed by concentration under reduced pressure. This afforded ((1S,3R,4S)-3-amino-4-hydroxycyclopentyl)((R)-2-((R)-1-(6-chloro-3′-ethylbiphenyl-2-yl)-1-hydroxypent-4-enyl)morpholino)methanone which was purified by reverse phase HPLC. LC-MS t_{R}=2.52 min, (m/z) 513.2 (M+H⁺).

### Example 26

The following compounds were prepared following procedures analogous to those described in Example 25: I-221A, I-224A, I-226A, I-226B, I-227A, I-229A, I-230A, I-240A, I-240B, I-244A, I-249A, I-250A, I-253A, I-254A, I-255A, I-256A, I-257A, I-261A, I-278A, I-281A, I-283A, I-284A, I-292A, I-292B, I-294A, I-295A, I-295B, I-295C, I-296A, I-296B, I-299A, I-305A, I-313A, I-321A, I-326A, I-326B, I-327A, I-334A, I-335A, I-336A.

### Example 27

### (3-(aminomethyl)phenyl)(4-(1-(3′-ethyl-6-fluorobiphenyl-2-yl)-1-hydroxy-5-methoxypentyl)piperidin-1-yl)methanone (I-242A)

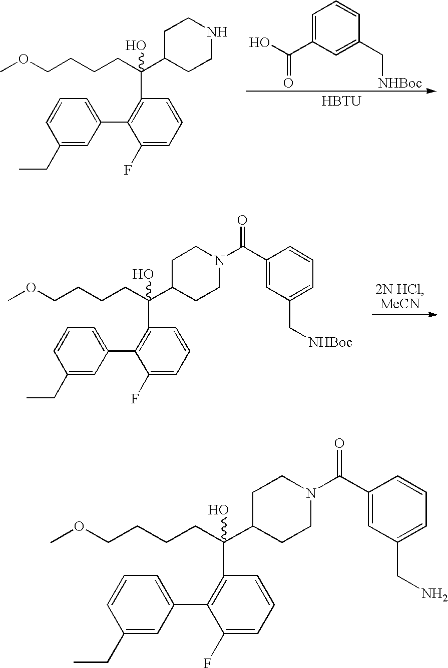

### Step 1. tert-butyl 3-(4-(1-(3′-ethyl-6-fluorobiphenyl-2-yl)-1-hydroxy-5-methoxypentyl)piperidine-1-carbonyl)benzylcarbamate

A solution of 1-(3′-ethyl-6-fluorobiphenyl-2-yl)-5-methoxy-1-piperidin-4-yl)pentan-1-ol (15 mg, 0.038 mmol) in 0.3 mL of DMF at 25° C. was treated with 33-((tert-butoxycarbonylamino)methyl)benzoic acid (11 mg, 0.042 mmol), i-Pr₂NEt (0.03 mL, 0.17 mmol), HBTU (16 mg, 0.042 mmol) and HOBt (6 mg, 0.042 mmol). After 20 h, H₂O was added and the mixture was extracted with EtOAc. The organic extracts were washed (1N HCl, 1N NaOH, H₂O, brine), dried (Na₂SO₄), concentrated under reduced pressure, and subjected to flash chromatography to provide tert-butyl 3-(4-(1-(3′-ethyl-6-fluorobiphenyl-2-yl)-1-hydroxy-5-methoxypentyl)piperidine-1-carbonyl)benzylcarbamate as a colorless oil (10 mg, 42%). MS (m/z) 633.3 (M+H⁺).

### Step 2. (3-(aminomethyl)phenyl)(4-(1-(3′-ethyl-6-fluorobiphenyl-2-yl)-1-hydroxy-5-methoxypentyl)piperidin-1-yl)methanone

A solution of tert-butyl 3-(4-(1-(3′-ethyl-6-fluorobiphenyl-2-yl)-1-hydroxy-5-methoxypentyl)piperidine-1-carbonyl)benzylcarbamate (10 mg, 0.016 mmol) in 1 mL of CH₃CN at 25° C. was treated with 1 mL of aqueous 2N HCl. After 24 h, the mixture was concentrated under reduced pressure to provide (3-(aminomethyl)phenyl)(4-(1-(3′-ethyl-6-fluorobiphenyl-2-yl)-1-hydroxy-5-methoxypentyl)piperidin-1-yl)methanone as a white solid (8 mg, quantitative). MS (m/z) 533.3 (M+H⁺).

The following piperidines were prepared following procedures analogous to those described above by using the indicated acid in place of 3-[({[(1,1-dimethylethyl)oxy]carbonyl}amino)methyl]benzoic acid in Step 1:

| Structure | Name | Step 1 Acid |
|---|---|---|
| 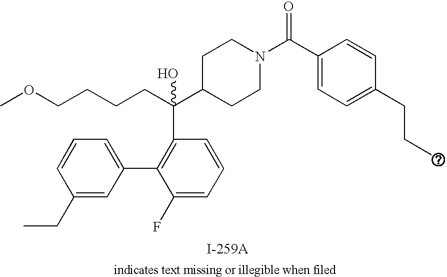 | 1-(1-{[4-(2- aminoethyl)phenyl]carbonyl}- 4-piperidinyl)-1-(3′-ethyl-6- fluoro-2-bephenylyl)-5- (methyloxy)-1-pentanol GSK1711463A | 4-[2-({[(1,1-dimethyl- ethyl)oxy]carbonyl}amino)ethyl]benzoic acid |

The following are compounds of the invention:

| | | Synthetic | LC_MS | | Selected 1H |
|---|---|---|---|---|---|
| | | Method | Method 1 | Mass | NMR |
| Cpd. No. | Name | Example No. | t_{R} (min) | observed | resonances^{a} |
| I-1A | ((1R,3S)-3-aminocyclopentyl)((R)-3-(1-(2- | 5 | 1.4 | 465 | 7.59-7.54 (m, 1H), |
| | phenylphenyl)-1-hydroxy-5- | | | | 7.18-6.92 (m, 7H), |
| | methoxypentyl)piperidin-1-yl)methanone | | | | 6.81-6.76 (m, 1H), |
| | | | | | 4.42 (d, J = 11.7 Hz, |
| | | | | | 0.5H), 4.23 (t, J = 12.6 Hz, |
| | | | | | 0.5 Hz), |
| | | | | | 3.83-3.72 (m, 1H), |
| | | | | | 3.55-3.45 (m, 1H), |
| | | | | | 3.17-3.09 (m, 5H), |
| | | | | | 2.88-2.70 (m, 1H), |
| | | | | | 2.37-2.17 (m, 1H), |
| | | | | | 2.03-0.70 (m, 18H). |
| I-2A | ((S)-3-aminopyrrolidin-1-yl)((R)-3-(1-(2- | 11 | 1.32 | 466, | 7.53 (d, J = 7.9 Hz, |
| | phenylphenyl)-1-hydroxy-5- | | | 448, | 1H), 7.17-6.98 (m, |
| | methoxypentyl)piperidin-1-yl)methanone | | | 488 | 7H), 6.79-6.76 (m, |
| | | | | | 1H), 3.64-3.45 (m, |
| | | | | | 3H), 3.35 (dd, J = 11.7, |
| | | | | | 6.2 Hz, 0.5H), |
| | | | | | 3.22 (dd, J = 11.8, |
| | | | | | 4.8 Hz, 0.5 Hz), |
| | | | | | 3.14-2.85 (m, 8H), |
| | | | | | 2.54-2.47 (m, 1H), |
| | | | | | 2.40-2.34 (m, 1H), |
| | | | | | 2.07-1.98 (m, 1H), |
| | | | | | 1.76-1.69 (m, 1H), |
| | | | | | 1.5 |
| I-3A | ((1S,3R,4S)-3-amino-4- | 5 | 1.32 | 475 | 7.24 (d), 7.13 (m), |
| | hydroxycyclopentyl)((3R)-3-(1-(2- | | | | 6.92-6.81 (m), |
| | (cyclopropylmethoxy)phenyl)-1-hydroxy-5- | | | | 6.63-6.52 (m), 4.14 (m), |
| | methoxypentyl)piperidin-1-yl)methanone | | | | 3.97-3.90 (m), |
| | | | | | 3.71 (br s), 2.97 (m), |
| | | | | | 2.94 (m), 2.90 (m), |
| | | | | | .050 (br s), 0.30 (br |
| | | | | | m), 0.00 (br s). |
| I-3B | ((1S,3R,4R)-3-amino-4- | 5 | 1.27 | 475 | 7.23 (d), 7.15 (br |
| | hydroxycyclopentyl)((3R)-3-(1-(2- | | | | m), 6.92-6.83 (m), |
| | (cyclopropylmethoxy)phenyl)-1-hydroxy-5- | | | | 6.63-6.52 (m), |
| | methoxypentyl)piperidin-1-yl)methanone | | | | 4.15 (t), 3.86 (m), |
| | | | | | 3.72 (m), 3.61 (m), |
| | | | | | 3.54 (m), 2.97 (m), |
| | | | | | 2.95 (m), 2.91 (m), |
| | | | | | 0.50 (br s), 0.30 (m), |
| | | | | | 0.00 (br s). |
| I-4A | ((1R,3S)-3-aminocyclopentyl)((R)-3-((S)-1- | 4 | 1.38 | 481 | 0.92 (m), |
| | hydroxy-5-methoxy-1-(2- | | | | 1.0-1.8 (m), 1.8-2.2 (m), |
| | phenoxyphenyl)pentyl)piperidin-1-yl)methanone | | | | 2.40 (m), 2.63 (m), |
| | | | | | 2.94, 3.14 (m), |
| | | | | | 3.24 (s), 3.28 (t), 3.36, |
| | | | | | 3.44 (m), 3.68 (m), |
| | | | | | 3.96, 4.26 (m), |
| | | | | | 4.44, 4.86 (m), |
| | | | | | 6.80 (two d), 6.92 (m), |
| | | | | | 7.08-7.24 (m), |
| | | | | | 7.38 (m), 7.64 (m) |
| I-4B | ((1R,3R)-3-aminocyclopentyl)((R)-3-((S)-1- | 5 | 1.35 | 481 | 0.84-2.44 (m), |
| | hydroxy-5-methoxy-1-(2- | | | | 2.58 (dd), 2.96, |
| | phenoxyphenyl)pentyl)piperidin-1-yl)methanone | | | | 3.18 (dd), 3.24 (s), |
| | | | | | 3.26 (t), 3.40 (m), |
| | | | | | 3.64 (m), 3.88, 4.22 (d), |
| | | | | | 4.42, 4.84 (d), |
| | | | | | 6.80 (dd), 6.90 (m), |
| | | | | | 7.04-7.26 (m), 7.36 (m), |
| | | | | | 7.64 (d) |
| I-5A^{b} | ((1S,3R,4S)-3-amino-4- | 5 | | 481 | 1.05 (m, 1H), |
| | hydroxycyclopentyl)((R)-3-((S)-1-(2- | | | | 3.28 (m, 6H), |
| | phenylphenyl)-1-hydroxy-5- | | | | 3.41-3.95 (m, 3H), |
| | methoxypentyl)piperidin-1-yl)methanone | | | | 4.3-4.65 (m, 1H), |
| | | | | | 7.03 (m, 1H), |
| | | | | | 7.20 (m, 4H), |
| | | | | | 7.35 (m, 4H), |
| | | | | | 8.12 (brs, 1H) |
| I-6A | (3-aminopyrrolidin-1-yl)((R)-3-((S)-1-hydroxy-5- | 11 | 1.33 | 482, 504 | 7.65 (dd, 1H), |
| | methoxy-1-(2-phenoxyphenyl)pentyl)piperidin- | | | | 7.38-7.26 (m, 2H), |
| | 1-yl)methanone | | | | 7.18 (m, 1H), |
| | | | | | 7.23-7.03 (m, 2H), |
| | | | | | 6.98-6.85 (m, 2H), |
| | | | | | 6.78 (m, 1H), |
| | | | | | 4.06 (br d, 1H), |
| | | | | | 3.79 (br s, |
| | | | | | 1H), 3.64 (m, 2H), |
| | | | | | 2.74 (t, 1H), 2.22 (m, |
| | | | | | 3H), 1.82 (m, 3H), |
| | | | | | 0.89 (m, 1H) |
| I-7A | ((R)-3-((S)-1-(2-(o-tolyloxy)phenyl)-1-hydroxy-5- | 11 | 1.42 | 504 | 7.64 (dd, 1H), |
| | methoxypentyl)piperidin-1-yl)(3-aminoazetidin- | | | | 7.29 (d, 1H), |
| | 1-yl)methanone | | | | 7.18-7.11 (m, 2H), |
| | | | | | 7.09-7.02 (t, 2H), 6.74 (d, |
| | | | | | 1H), 6.53 (dd, 1H), |
| | | | | | 4.26 (m, 2H), |
| | | | | | 4.15 (d, 1H), |
| | | | | | 4.03 (m, 1H), |
| | | | | | 3.96-3.88 (m, 2H), |
| | | | | | 3.73 (d, 1H), 3.24 (s, |
| | | | | | 3H), 2.84 (t, 1H), |
| | | | | | 2.68 (dt, 1H), |
| | | | | | 2.40 (m, 2H), 2.23 (s, |
| | | | | | 3H), 1.90 (dt, 2H), |
| | | | | | 1.62 (td, 1H), |
| I-8A^{b} | ((S)-3-aminopyrrolidin-1-yl)((R)-3-((S)-1-(2- | 11 | | 484 | 1.02 (brs, 1H), |
| | ethyl-3-fluorophenyl)-1-hydroxy-5- | | | | 2.95 (m, 1H), 3.31 (s, |
| | methoxypentyl)piperidin-1-yl)methanone | | | | 3H), 6.99 (m, 1H), |
| | | | | | 7.13-7.47 (m, 7H) |
| I-9A | (3-(1-(2-(o-tolyloxy)phenyl)-1-hydroxy-5- | 1 | 1.38 | 489, | 7.83-7.78 (m, 1H), |
| | methoxypentyl)phenyl)(3-aminopyrrolidin-1- | | | 471, | 7.55-6.84 (m, 9H), |
| | yl)methanone | | | 511 | 6.32 (d, J = 7.6 Hz, |
| | | | | | 1H), 6.16 (m, 1H), |
| | | | | | 3.84-3.48 (m, 4H), |
| | | | | | 3.26 (t, J = 6.4 Hz, |
| | | | | | 2H), 3.17 (s, 3H), |
| | | | | | 2.71-2.62 (m, 1H), |
| | | | | | 2.24-2.17 (m, 2H), |
| | | | | | 2.08-2.02 (m, 2H), |
| | | | | | 1.77 (s, 3H), |
| | | | | | 1.53-1.37 (m, 3H), |
| | | | | | 1.19-1.12 (m, 1H). |
| I-10A | ((3R)-3-(1-(2-(2-cyclopropylethoxy)phenyl)-1- | 5 | 1.41 | 489 | 7.44 (d), 7.33 (m), |
| | hydroxy-5-methoxypentyl)piperidin-1- | | | | 7.17-7.03 (m), |
| | yl)((1S,3R,4S)-3-amino-4- | | | | 6.90-6.74 (m), 4.31 (m), |
| | hydroxycyclopentyl)methanone | | | | 4.13 (m), 3.90 (m), |
| | | | | | 3.39 (m), 3.13 (m), |
| | | | | | 3.10 (m), 3.08 (s), |
| | | | | | 0.73 (m), 0.35 (br |
| | | | | | m), 0.00 (br m). |
| I-10B | ((3R)-3-(1-(2-(2-cyclopropylethoxy)phenyl)-1- | 5 | 1.33 | 488 | 7.44 (d), 7.33 (m), |
| | hydroxy-5-methoxypentyl)piperidin-1- | | | | 7.11-7.04 (m), |
| | yl)((1S,3R,4R)-3-amino-4- | | | | 6.86-6.75 (m), 3.16 (m), |
| | hydroxycyclopentyl)methanone | | | | 3.08 (m), 0.72 (m), |
| | | | | | 0.35 (m), 0.01 (m) |
| I-11A | ((1R,3S)-3-aminocyclopentyl)((3R)-3-((1S)-1- | 5 | | 479 | 1.02 (m, 1H), |
| | hydroxy-5-methoxy-1-(2′-methylbiphenyl-2- | | | | 2.00 (s, 3H), 2.42 (m, |
| | yl)pentyl)piperidin-1-yl)methanone | | | | 1H), 3.94 (m, 1H), |
| | | | | | 3.28 (s, 3H), |
| | | | | | 3.64 (m, 1H), 3.94 (m, |
| | | | | | 1H), 4.48&4.56 (m, |
| | | | | | 1H), 6.92 (m, 1H), |
| | | | | | 7.10-7.49 (m, 6H), |
| | | | | | 7.75 (m, 1H) |
| I-12A | ((1S,3R,4R)-3-amino-4- | 5 | 1.4 | 489 | 7.57 (d), 7.48 (t), |
| | hydroxycyclopentyl)((3R)-3-(1-(2- | | | | 725-716 (m), |
| | (cyclobutylmethoxy)phenyl)-1-hydroxy-5- | | | | 6.98-6.86 (m), 4.45 (q), |
| | methoxypentyl)piperidin-1-yl)methanone | | | | 4.25-4.18 (m), |
| | | | | | 3.3 (m), 3.22 (s), |
| | | | | | 0.80 (br s). |
| I-12B | ((1S,3R,4S)-3-amino-4- | 5 | 1.49 | 489 | 7.58 (d), 7.46 (m), |
| | hydroxycyclopentyl)((3R)-3-(1-(2- | | | | 7.26-7.16 (m), |
| | (cyclobutylmethoxy)phenyl)-1-hydroxy-5- | | | | 7.97-6.86 (m), 4.46 (t), |
| | methoxypentyl)piperidin-1-yl)methanone | | | | 3.29 (m), 3.26 (m), |
| | | | | | 3.22 (m), 0.78 (br |
| | | | | | s). |
| I-13A | ((1S,3R,4S)-3-amino-4- | 5 | 1.38 | 489 | 7.57 (d), 7.46 (d), |
| | hydroxycyclopentyl)((3R)-3-(1-(2- | | | | 7.22 (m), 7.18 (m), |
| | (cyclopentyloxy)phenyl)-1-hydroxy-5- | | | | 6.93 (m), 6.87 (m), |
| | methoxypentyl)piperidin-1-yl)methanone | | | | 4.47 (m), |
| | | | | | 4.32-4.22 (m), 4.04 (m), |
| | | | | | 3.94 (m), 3.29 (m), |
| | | | | | 3.26 (m), 3.23 (m), |
| | | | | | 0.79 (br s) |
| I-14A | (2-aminopyridin-4-yl)((R)-3-((S)-1-hydroxy-5- | 9 | 1.4 | 490 | 0.92 (m), |
| | methoxy-1-(2-phenoxyphenyl)pentyl)piperidin- | | | | 1.04-2.40 (m), 2.64, 2.82 (m), |
| | 1-yl)methanone | | | | 2.96 (m), 3.18 (s), |
| | | | | | 3.44, 3.68 (d), |
| | | | | | 4.44 (d), 6.56-6.98 (m), |
| | | | | | 7.12 (m), 7.22 (m), |
| | | | | | 7.38 (m), 7.84 (m), |
| | | | | | 7.84 (m) |
| I-15A | (6-aminopyridin-3-yl)((R)-3-((S)-1-hydroxy-5- | 9 | 1.38 | 490 | 0.92 (m), |
| | methoxy-1-(2-phenoxyphenyl)pentyl)piperidin- | | | | 1.02-1.78 (m), 1.90 (m), |
| | 1-yl)methanone | | | | 2.04-2.48 (m), 3.24 (s), |
| | | | | | 3.60 (m), 3.88, |
| | | | | | 4.40 (m), 6.80 (m), |
| | | | | | 6.98 (m), 7.12 (m), |
| | | | | | 7.20 (m), 7.38 (m), |
| | | | | | 7.60-7.80 (m), 7.86 (d) |
| I-16A | ((1S,3R,4S)-3-amino-4- | 3 | 1.42 | 491 | 7.62 (d), 7.50 (br s), |
| | hydroxycyclopentyl)((3R)-3-(1-hydroxy-5- | | | | 7.27 (m), 7.20 (m), |
| | methoxy-1-(2- | | | | 7.00-6.89 (m), |
| | (neopentyloxy)phenyl)pentyl)piperidin-1- | | | | 4.47 (t), 4.27 (m), |
| | yl)methanone | | | | 3.98 (m), 3.31 (m), |
| | | | | | 3.22 (m), 2.94 (m), |
| | | | | | 2.64 (t), 1.06 (m) |
| I-17A | (3-aminocyclohexyl)((R)-3-((S)-1-hydroxy-5- | 5 | 1.4 | 495 | 0.92 (m), |
| | methoxy-1-(2-phenoxyphenyl)pentyl)piperidin- | | | | 1.2-1.9 (m), 2.0-2.2 (m), |
| | 1-yl)methanone | | | | 2.40 (m), |
| | | | | | 2.7-3.0 (m), 3.22 (s), |
| | | | | | 3.24 (t), 3.60, 3.90 (m), |
| | | | | | 4.20, 4.42 (m), |
| | | | | | 6.72, 6.80 (m), |
| | | | | | 6.92, 7.0 (m), |
| | | | | | 7.04-7.38 (m), 7.65, |
| | | | | | 7.78 (m) |
| I-17B | (3-aminocyclohexyl)(3-((S)-1-hydroxy-5- | 5 | 1.47 | 495 | 0.82-1.70 (m), |
| | methoxy-1-(2-phenoxyphenyl)pentyl)piperidin- | | | | 1.72-2.20 (m), 2.38 (m), |
| | 1-yl)methanone | | | | 2.58 (m), 2.84 (m), |
| | | | | | 3.10 (m), 3.24 (s), |
| | | | | | 3.26 (t), 3.86 (m), |
| | | | | | 4.20 (m), 4.42 (m), |
| | | | | | 6.80 (m), |
| | | | | | 6.86 (m), 7.06-7.24 (m), |
| | | | | | 7.36 (m), 7.64 (d) |
| I-17C | ((1S)-3-aminocyclohexyl)((R)-3-((S)-1-hydroxy- | 5 | 1.4 | 495 | 0.82-2.04 (m), |
| | 5-methoxy-1-(2- | | | | 2.18 (m), 2.36 (m), 2.42, |
| | phenoxyphenyl)pentyl)piperidin-1-yl)methanone | | | | 2.58 (m), 2.82 (m), |
| | | | | | 2.90, 3.06 (m), |
| | | | | | 3.24 (s), 3.26 (t), 3.90, |
| | | | | | 4.22 (m), 4.44, |
| | | | | | 4.86 (m), 6.80 (m), |
| | | | | | 6.88 (m), 7.04-7.24 (m), |
| | | | | | 7.36 (m), 7.64 (d) |
| I-18A | ((R)-3-((S)-1-(2-(o-tolyloxy)phenyl)-1-hydroxy-5- | 5 | 1.49 | 495 | 0.80-1.98 (m), |
| | methoxypentyl)piperidin-1-yl)((1R,3S)-3- | | | | 2.08 (m), 2.26 (m), 2.42, |
| | aminocyclopentyl)methanone | | | | 2.60 (m), 2.94, |
| | | | | | 3.14 (m), 3.24 (s), |
| | | | | | 3.26 (t), 3.38, 3.42 (m), |
| | | | | | 3.68 (m), 3.94, |
| | | | | | 4.24 (m), 4.44, 4.86 (m), |
| | | | | | 6.62-6.92 (m), |
| | | | | | 7.16-7.36 (m), 7.72 (m) |
| I-19A | ((1R,3S)-3-aminocyclopentyl)((R)-3-((S)-1- | 5 | | 479 | 0.92 (m, 1H), |
| | hydroxy-5-methoxy-1-(3′-methylbiphenyl-2- | | | | 2.06 (m, 3H), 2.34 (m, |
| | yl)pentyl)piperidin-1-yl)methanone | | | | 3H), 2.53 (m, 1H), |
| | | | | | 3.01 (m, 1H), |
| | | | | | 3.28 (s, 3H), 3.68 (m, |
| | | | | | 1H), 3.96 (m, 1H), |
| | | | | | 4.40 & 4.56 (m, |
| | | | | | 1H), 6.86-7.04 (m, |
| | | | | | 3H), 7.16-7.26 (m, |
| | | | | | 3H), 7.34 (m, 1H), |
| | | | | | 7.75 (m, 1H) |
| I-20A^{b} | ((1S,3R,4S)-3-amino-4- | 5 | | 495 | 1.03 (s, 1H), 2.35 & |
| | hydroxycyclopentyl)((R)-3-((S)-1-(2- | | | | 2.36 (s, 3H), |
| | phenylphenyl)-1-hydroxy-5- | | | | 3.30 (d, 3H), |
| | methoxypentyl)piperidin-1-yl)methanone | | | | 3.61-3.88 (m, 1H), |
| | | | | | 4.42 (m, 1H), |
| | | | | | 7.00 (m, 4H), |
| | | | | | 7.21 (m, 2H), 7.35 (m, 2H) |
| I-21A | ((R)-3-aminopiperidin-1-yl)((R)-3-((S)-1- | 11 | 1.44 | 496 | 7.65 (dd, 1H), |
| | hydroxy-5-methoxy-1-(2- | | | | 7.36-7.28 (m, 2H), |
| | phenoxyphenyl)pentyl)piperidin-1-yl)methanone | | | | 7.18 (m, 1H), |
| | | | | | 7.13-7.03 (m, 2H), |
| | | | | | 6.95-6.87 (m, 2H), |
| | | | | | 6.78 (m, 1H), |
| | | | | | 3.97 (d, 1H), |
| | | | | | 3.59 (m, 1H), |
| | | | | | 2.99 (m, 1H), |
| | | | | | 2.80-2.52 (m, 2H), |
| | | | | | 0.89 (m, 1H) |
| I-22A | ((R)-3-((S)-1-(2-(o-tolyloxy)phenyl)-1-hydroxy-5- | 10 | 1.42 | 496 | 7.64 (dd, 1H), |
| | methoxypentyl)piperidin-1-yl)((S)-3- | | | | 7.28 (d, 1H), |
| | aminopyrrolidin-1-yl)methanone | | | | 7.18-7.12 (m, 2H), 7.05 (t, |
| | | | | | 2H), 6.74 (d, 1H), |
| | | | | | 6.55 (d, 1H), 4.09 (d, |
| | | | | | 1H), 3.82 (m, 1H), |
| | | | | | 3.74-3.62 (m, 2H), |
| | | | | | 3.45 (m, 3H), 2.81 (t, |
| | | | | | 1H), 2.68 (t, 1H), |
| | | | | | 2.41 (m, 2H), |
| | | | | | 2.26 (m, 1H), 2.24 (s, |
| | | | | | 3H), 1.90 (m, 2H), |
| | | | | | 1.62 (d, 1H), |
| | | | | | 0.98 (m, 1H) |
| I-22B | ((R)-3-((S)-1-(2-(o-tolyloxy)phenyl)-1-hydroxy-5- | 11 | 1.45 | 496 | 7.65 (dd, 1H), |
| | methoxypentyl)piperidin-1-yl)((R)-3- | | | | 7.28 (d, 1H), |
| | aminopyrrolidin-1-yl)methanone | | | | 7.18-7.12 (m, 2H), |
| | | | | | 7.08-7.03 (m, 2H), |
| | | | | | 6.73 (d, 1H), |
| | | | | | 6.56 (dd, 1H), |
| | | | | | 4.20 (d, 1H), |
| | | | | | 3.83 (m, 1H), |
| | | | | | 3.74-3.65 (m, 2H)m |
| | | | | | 3.55 (m, 1H), |
| | | | | | 3.38 (m, 2H), 2.81 (t, |
| | | | | | 1H), 2.68 (t, 1H), |
| | | | | | 2.42 (m, 2H), |
| | | | | | 1.62 (m, 1H), |
| | | | | | 0.97 (m, 1H) |
| I-23A | ((R)-3-((S)-1-(2-(m-tolyloxy)phenyl)-1-hydroxy- | 11 | 1.44 | 496 | 7.69 (dd, 1H), |
| | 5-methoxypentyl)piperidin-1-yl)((S)-3- | | | | 7.24 (m, 2H), |
| | aminopyrrolidin-1-yl)methanone | | | | 7.14 (m, 1H), |
| | | | | | 6.96 (m, 1H), |
| | | | | | 6.83 (d, 1H), |
| | | | | | 6.76 (m, 2H), |
| | | | | | 4.02 (d, 1H), 3.69 (d, |
| | | | | | 1H), 3.09 (m, 1H), |
| | | | | | 2.76 (t, 1H), 2.61 (td, |
| | | | | | 1H), 2.33 (s, 3H), |
| | | | | | 2.04 (m, 1H), |
| | | | | | 1.87 (m, 1H), |
| | | | | | 0.93 (m, 1H). |
| I-24A | ((R)-3-((S)-1-(2-(o-tolyloxy)phenyl)-1-hydroxy-5- | 11 | 1.49 | 496 | 7.64 (dd, 1H), |
| | methoxypentyl)piperidin-1-yl)(2- | | | | 7.29 (m, 1H), |
| | (aminomethyl)azetidin-1-yl)methanone | | | | 7.19-7.12 (m, 2H), |
| | | | | | 7.09-7.03 (m, 2H), |
| | | | | | 6.73 (m, 1H), |
| | | | | | 6.55 (m, 1H), |
| | | | | | 4.50 (m, 1H), |
| | | | | | 3.52 (m, 1H), |
| | | | | | 2.07 (m, 1H), |
| | | | | | 1.61 (d, 1H), |
| | | | | | 0.97 (m, 1H). |
| I-25A | ((1S,3R,4S)-3-amino-4- | 5 | 1.37 | 497 | 0.90 (m), 1.16 (m), |
| | hydroxycyclopentyl)((R)-3-((S)-1-hydroxy-5- | | | | 1.22-1.98 (m), |
| | methoxy-1-(2-phenoxyphenyl)pentyl)piperidin- | | | | 2.04-2.38 (m), 2.44, |
| | 1-yl)methanone | | | | 2.62 (m), 2.96, 3.12 (m), |
| | | | | | 3.22 (s), 3.24 (t), |
| | | | | | 3.36 (m), 3.48 (m), |
| | | | | | 3.92, 4.24 (m), |
| | | | | | 4.26 (m), 4.42, 4.84 (m), |
| | | | | | 6.80 (m), 6.92 (m), |
| | | | | | 7.06-7.24 (m), |
| | | | | | 7.36 (m), 7.64 (d) |
| I-25B | ((1S,3R,4R)-3-amino-4- | 5 | 1.33 | 497 | 0.84-2.32 (m), 2.42, |
| | hydroxycyclopentyl)((R)-3-((S)-1-hydroxy-5- | | | | 2.62 (m), 2.96, |
| | methoxy-1-(2-phenoxyphenyl)pentyl)piperidin- | | | | 3.12 (m), 3.24 (s), |
| | 1-yl)methanone | | | | 3.26 (t), 3.40 (m), 3.42, |
| | | | | | 3.56 (m), 3.92, |
| | | | | | 4.22 (m), 4.16 (m), 4.42, |
| | | | | | 4.84 (m), 6.80 (m), |
| | | | | | 6.90 (m), |
| | | | | | 7.04-7.24 (m), 7.36 (m), |
| | | | | | 7.64 (m) |
| I-26A | ((1R,3S)-3-aminocyclopentyl)((R)-3-((S)-1- | 5 | | 479 | 0.92 (m, 1H), |
| | hydroxy-5-methoxy-1-(4′-methylbiphenyl-2- | | | | 2.38 (s, 3H), 3.04 (m, |
| | yl)pentyl)piperidin-1-yl)methanone | | | | 1H), 3.28 (s, 3H), |
| | | | | | 3.66 (m, 1H), |
| | | | | | 4.00 (m, 1H), 4.45& |
| | | | | | 4.60 (m, 1H), |
| | | | | | 6.90-7.45 (m, 7H), 7.74 (m, |
| | | | | | 1H) |
| I-27A^{b} | ((R)-3-((R)-(3-methoxypropoxy)(2-(o- | 5 | | 497 | 2.25 (s, 3H), |
| | tolyloxy)phenyl)methyl)piperidin-1- | | | | 3.31 (s, 3H), 4.06 (d, 1H), |
| | yl)((1S,3R,4S)-3-amino-4- | | | | 4.25 (d, 1H), |
| | hydroxycyclopentyl)methanone | | | | 4.40 (m, 1H), |
| | | | | | 4.60 (m, 1H), 6.61 (t, 1H), |
| | | | | | 6.77 (d, 1H), |
| | | | | | 7.05-7.2 (m, 5H), |
| | | | | | 7.4 (m, 1H), |
| | | | | | 8.09 (brs, 2H) |
| I-27B^{bc} | ((R)-3-((S)-(3-methoxypropoxy)(2-(o- | 5 | | 497 | 1.38 (m, 2H), |
| | tolyloxy)phenyl)methyl)piperidin-1- | | | | 2.24 (s, 3H), 2.72 (m, 1H), |
| | yl)((1S,3R,4S)-3-amino-4- | | | | 3.08 (m, 1H), |
| | hydroxycyclopentyl)methanone | | | | 3.29 (s, 3H), 4.06 (d, 1H), |
| | | | | | 6.63 (t, 1H), |
| | | | | | 6.78 (d, 1H), |
| | | | | | 7.05-7.18 (m, 5H), |
| | | | | | 7.42 (m, 1H), |
| | | | | | 8.09 (brs, 2H) |
| I-28A | ((R)-3-((S)-1-(2-(3-fluorophenoxy)phenyl)-1- | 5 | 1.44 | 499 | 0.84-2.36 (m), 2.42, |
| | hydroxy-5-methoxypentyl)piperidin-1- | | | | 2.62 (dd), 2.94, |
| | yl)((1R,3S)-3-aminocyclopentyl)methanone | | | | 3.14 (dd), 3.24 (s), |
| | | | | | 3.26 (t), 3.36, |
| | | | | | 3.44 (m), 3.68 (m), 3.96, |
| | | | | | 4.26 (d), 4.44, |
| | | | | | 4.86 (d), 6.62-6.92 (m), |
| | | | | | 7.18-7.36 (m), |
| | | | | | 7.72 (m) |
| I-29^{b} | ((1S,3R,4S)-3-amino-4- | 5 | | 499 | 1.03 (s, 1H), 3.28 & |
| | hydroxycyclopentyl)((R)-3-((S)-1-(2-phenyl-3- | | | | 3.30 (s, 3H), |
| | fluorophenyl)-1-hydroxy-5- | | | | 3.45-3.88 (m, 1H), |
| | methoxypentyl)piperidin-1-yl)methanone | | | | 4.22-4.60 (m, 2H), |
| | | | | | 7.01 (m, 1H), |
| | | | | | 7.18 (m, 3H), |
| | | | | | 7.35 (m, 4H), |
| | | | | | 8.12 (brs, 2H), |
| I-30A | ((S)-3-aminopyrrolidin-1-yl)((R)-3-((S)-1-(3- | 11 | 1.33 | 500 | 7.52 (dt, 1H), |
| | fluoro-2-phenoxyphenyl)-1-hydroxy-5- | | | | 7.31-7.27 (m, 2H), |
| | methoxypentyl)piperidin-1-yl)methanone | | | | 7.26-7.21 (m, 1H), |
| | | | | | 7.14 (m, 1H), 7.02 (t, |
| | | | | | 1H), 6.82 (d, 2H), |
| | | | | | 3.99 (d, 1H), |
| | | | | | 3.83 (m, 1H), |
| | | | | | 3.61 (m, 2H), 2.71 (t, |
| | | | | | 1H), 2.59 (td, 1H), |
| | | | | | 1.52 (dt, 1H), |
| | | | | | 1.05 (m, 1H), |
| | | | | | 0.81 (m, 1H). |
| I-31A | ((R)-3-((S)-1-(2-(3-fluorophenoxy)phenyl)-1- | 11 | 1.4 | 500 | 7.70 (dd, 1H), |
| | hydroxy-5-methoxypentyl)piperidin-1-yl)((S)-3- | | | | 7.33 (m, 1H), |
| | aminopyrrolidin-1-yl)methanone | | | | 7.27 (m, 1H), |
| | | | | | 7.18 (m, 1H), |
| | | | | | 6.89 (dd, 1H), |
| | | | | | 6.83 (td, 1H), |
| | | | | | 6.75 (dd, 1H), |
| | | | | | 6.68 (dd, 1H), |
| | | | | | 4.05 (d, 1H), |
| | | | | | 3.83 (m, 1H), |
| | | | | | 3.70-3.60 (m, 2H), 2.76 (t, |
| | | | | | 1H), 2.64 (td, 1H), |
| | | | | | 1.59 (dt, 1H), |
| | | | | | 0.89 (m, 1H). |
| I-32A | ((R)-3-((S)-1-(2-(4-fluorophenoxy)phenyl)-1- | 11 | 1.38 | 500 | 7.66 (dd, 1H), |
| | hydroxy-5-methoxypentyl)piperidin-1-yl)((S)-3- | | | | 7.20 (m, 1H), |
| | aminopyrrolidin-1-yl)methanone | | | | 7.13-7.07 (m, 3H), |
| | | | | | 6.97-6.93 (m, 2H), |
| | | | | | 6.75 (d, 1H), 4.07 (d, |
| | | | | | 1H), 3.82 (m, 1H), |
| | | | | | 3.72-3.61 (m, 2H), |
| | | | | | 2.79 (t, 1H), 2.65 (td, |
| | | | | | 1H), 1.61 (d, 1H), |
| | | | | | 0.93 (m, 1H). |
| I-33A | ((1S,3R,4S)-3-amino-4-hydroxycyclopentyl)(3- | 5 | 1.47 | 503 | 7.59 (d), 7.48 (t), |
| | (1-(2-(cyclopentylmethoxy)phenyl)-1-hydroxy-5- | | | | 7.26 (m), 7.19 (m), |
| | methoxypentyl)piperidin-1-yl)methanone | | | | 6.96 (m), 6.90 (m), |
| | | | | | 4.48 (t), 3.53 (t), |
| | | | | | 3.30 (m), 3.23 (m), |
| | | | | | 2.62 (t), 0.79 (br s) |
| I-34A | ((R)-3-((S)-1-(2-(o-tolyloxy)phenyl)-1-hydroxy-5- | 9 | 1.45 | 504 | 0.88-2.0 (m), |
| | methoxypentyl)piperidin-1-yl)(6-aminopyridin-3- | | | | 2.22 (s), 2.40 (m), |
| | yl)methanone | | | | 3.24 (s), 3.64 (m), |
| | | | | | 6.60 (d), 6.74 (d), |
| | | | | | 6.90-7.20 (m), 7.32 (d), |
| | | | | | 7.62-7.90 (m) |
| I-35A | ((R)-3-((S)-1-(2-(o-tolyloxy)phenyl)-1-hydroxy-5- | 8 | 1.45 | 504 | 0.88-2.0 (m), |
| | methoxypentyl)piperidin-1-yl)(2-aminopyridin-4- | | | | 2.26 (s), 2.82-3.04 (m), |
| | yl)methanone | | | | 3.22, 3.24 (s), |
| | | | | | 3.46 (d), 3.78, 4.48 (d), |
| | | | | | 6.52-6.86 (m), |
| | | | | | 7.04-7.20 (m), 7.32 (m), |
| | | | | | 7.60, 7.66 (d), 7.80, |
| | | | | | 7.90 (d) |
| I-36A | (3-(1-(2-(o-tolyloxy)phenyl)-1-hydroxy-5- | 2 | 1.36 | 487, | ND |
| | methoxypentyl)phenyl)((3R,4S)-3-amino-4- | | | 505, | |
| | hydroxypyrrolidin-1-yl)methanone | | | 527 | |
| I-37A | ((R)-3-((S)-1-(2-(o-tolyloxy)phenyl)-1-hydroxy-5- | 5 | 1.51 | 509 | 0.84-1.80 (m), |
| | methoxypentyl)piperidin-1-yl)(3- | | | | 1.9 (m), 2.22 (s), |
| | aminocyclohexyl)methanone | | | | 2.40 (m), 2.58 (m), 2.82, |
| | | | | | 2.92 (m), 3.28 (m), |
| | | | | | 3.24 (s), 3.26 (t), |
| | | | | | 3.92 (m), 4.20, |
| | | | | | 4.44 (m), 6.56-6.78 (m), |
| | | | | | 7.00-7.36 (m), |
| | | | | | 7.64 (d) |
| I-37B | ((R)-3-((S)-1-(2-(o-tolyloxy)phenyl)-1-hydroxy-5- | 5 | 1.45 | 553 | 0.86-2.02 (m), 2.22, |
| | methoxypentyl)piperidin-1-yl)((1R,3S)-3- | | | | 2.24 (s), 2.60 (dd), |
| | aminocyclohexyl)methanone | | | | 2.80-3.00 (m), 3.24, |
| | | | | | 3.26 (s), 3.26 (t), |
| | | | | | 3.94, 4.22 (d), 4.46, |
| | | | | | 4.86 (d), 6.58 (d), |
| | | | | | 6.74 (d), |
| | | | | | 7.04-7.20 (m), 7.28 (m), |
| | | | | | 7.64 (d) |
| I-38A | ((R)-3-((S)-1-(2-(2-ethylphenoxy)phenyl)-1- | 11 | 1.5 | 510 | 7.64 (dd, 1H), |
| | hydroxy-5-methoxypentyl)piperidin-1-yl)((S)-3- | | | | 7.31 (dd, 1H), |
| | aminopyrrolidin-1-yl)methanone | | | | 7.19-7.03 (m, 4H), |
| | | | | | 6.73 (d, 1H), 6.56 (d, |
| | | | | | 1H), 4.10 (d, 1H), |
| | | | | | 3.81 (m, 1H), 3.23 (s, |
| | | | | | 3H), 2.82 (t, 1H), |
| | | | | | 1.63 (d, 1H), 1.19 (t, |
| | | | | | 3H), 0.97 (m, 1H). |
| I-39A | ((R)-3-((S)-1-(2-(o-tolyloxy)phenyl)-5-ethoxy-1- | 11 | 1.48 | 510 | 7.64 (dd, 1H), |
| | hydroxypentyl)piperidin-1-yl)((S)-3- | | | | 7.29 (d, 1H), |
| | aminopyrrolidin-1-yl)methanone | | | | 7.18-7.12 (m, 2H), 7.06 (t, |
| | | | | | 2H), 6.74 (d, 1H), |
| | | | | | 6.55 (d, 1H), 4.09 (d, |
| | | | | | 1H), 3.81 (m, 1H), |
| | | | | | 3.73-3.61 (m, 2H), |
| | | | | | 2.81 (t, 1H), 2.67 (t, |
| | | | | | 1H), 2.23 (s, 3H), |
| | | | | | 1.63 (dt, 1H), 1.11 (t, |
| | | | | | 3H), 0.98 (m, 1H) |
| I-40A | ((R)-3-((S)-1-(2-(o-tolyloxy)phenyl)-1-hydroxy-5- | 11 | 1.4 | 510 | 7.65 (d, 1H), 7.29 (d, |
| | methoxypentyl)piperidin-1-yl)(3- | | | | 1H), 7.18-7.12 (m, |
| | (methylamino)pyrrolidin-1-yl)methanone | | | | 2H), 7.08 (t, 2H), |
| | | | | | 6.74 (d, 1H), 6.56 (d, |
| | | | | | 1H), 4.10 (d, 1H), |
| | | | | | 3.23 (s, 3H), 2.82 (q, |
| | | | | | 1H), 2.42 (m, 2H), |
| | | | | | 2.24 (s, 3H), 1.62 (d, |
| | | | | | 1H), 0.97 (m, 1H). |
| I-41A | ((R)-3-((S)-1-(2-(o-tolyloxy)phenyl)-1-hydroxy-5- | 5 | 1.44 | 511 | 0.94 (m), |
| | methoxypentyl)piperidin-1-yl)((1S,3R,4S)-3- | | | | 1.10-2.10 (m), 2.22, 2.24 (s), |
| | amino-4-hydroxycyclopentyl)methanone | | | | 2.44, 2.64 (m), |
| | | | | | 2.98, 3.16 (m), |
| | | | | | 3.24 (s), 3.26 (t), |
| | | | | | 3.48 (m), 3.94 (m), |
| | | | | | 4.24 (m), 4.44 (m), |
| | | | | | 6.58 (m), 6.72 (d), |
| | | | | | 7.04 (m), 7.16 (m), |
| | | | | | 7.26 (m), 7.64 (d) |
| I-41B | ((R)-3-((S)-1-(2-(o-tolyloxy)phenyl)-1-hydroxy-5- | 5 | 1.41 | 511 | 0.94 (m), |
| | methoxypentyl)piperidin-1-yl)((1S,3R,4R)-3- | | | | 1.06-1.82 (m), 1.94 (m), |
| | amino-4-hydroxycyclopentyl)methanone | | | | 2.06 (m), 2.22, 2.24 (s), |
| | | | | | 2.3 (m), 2.44, |
| | | | | | 2.64 (m), 2.98, 3.16 (m), |
| | | | | | 3.24 (s), 3.26 (t), |
| | | | | | 3.38 (m), 3.44, |
| | | | | | 3.58 (m), 3.96, 4.24 (m), |
| | | | | | 4.06, 4.18 (m), |
| | | | | | 4.44, 4.86 (m), |
| | | | | | 6.58 (m), 6.72 (m), |
| | | | | | 7.02-7.20 (m), 7.28 (m), |
| | | | | | 7.64 (d) |
| I-42A | ((S)-3-aminopyrrolidin-1-yl)((3R)-3-((1S)-1- | 11 | | 480 | 1.00 (m, 1H), |
| | hydroxy-5-methoxy-1-(2′-methylbiphenyl-2- | | | | 1.86 (m, 2H), 2.03 (s, |
| | yl)pentyl)piperidin-1-yl)methanone | | | | 3H), 2.20 (m, 1H), |
| | | | | | 2.58 (m, 2H), |
| | | | | | 3.06 (m, 2H), 3.28 (s, |
| | | | | | 3H), 3.44 (m, 1H), |
| | | | | | 3.68 (m, 2H), |
| | | | | | 3.72 (m, 1H), 6.94 (m, |
| | | | | | 1H), 7.08-7.26 (m, |
| | | | | | 5H), 7.36 (m, 1H), |
| | | | | | 7.74 (m, 1H) |
| I-43A | ((R)-3-((S)-1-(2-(m-tolyloxy)phenyl)-1-hydroxy- | 5 | 1.47 | 511 | 0.92 (m), |
| | 5-methoxypentyl)piperidin-1-yl)((1S,3R,4S)-3- | | | | 1.06-2.00 (m), 2.28 (s), 2.44, |
| | amino-4-hydroxycyclopentyl)methanone | | | | 2.62 (dd), 2.94, |
| | | | | | 3.12 (dd), 3.24 (s), |
| | | | | | 3.26 (t), 3.48 (m), |
| | | | | | 3.92 (d), 4.26 (m), |
| | | | | | 4.44 (d), |
| | | | | | 6.70-6.92 (m), 7.08-7.24 (m), |
| | | | | | 7.64 (d) |
| I-44A | ((1S,3R,4S)-3-amino-4- | 5 | 1.34, | 511 | 7.60-6.95 (m), |
| | hydroxycyclopentyl)((3R)-3-(1-(2- | | 1.39 | | 5.09 (q), 4.45 (br d), |
| | (benzyloxy)phenyl)-1-hydroxy-5- | | | | 4.35 (m), 4.08 (q), |
| | methoxypentyl)piperidin-1-yl)methanone | | | | 3.94 (br d), 3.31 (m), |
| | | | | | 3.22 (m). |
| I-45A | ((R)-3-((S)-1-(2-(o-tolyloxy)phenyl)-1-hydroxy-5- | 11 | 1.41 | 512 | 7.64 (dd, 1H), |
| | methoxypentyl)piperidin-1-yl)((3R,4S)-3-amino- | | | | 7.29 (d, 1H), |
| | 4-hydroxypyrrolidin-1-yl)methanone | | | | 7.18-7.11 (m, 2H), |
| | | | | | 7.09 (m, 2H), |
| | | | | | 6.75 (d, 1H), |
| | | | | | 6.53 (dd, 1H), |
| | | | | | 4.33 (m, 1H), |
| | | | | | 4.09 (d, 1H), 3.22 (s, |
| | | | | | 3H), 2.25 (s, 3H), |
| | | | | | 1.63 (dt, 1H), 1.11 (t, |
| | | | | | 3H), 0.98 (m, 1H) |
| I-46A | ((R)-3-((S)-1-(2-(3-fluorophenoxy)phenyl)-1- | 5 | 1.45 | 513 | 0.80-1.68 (m), |
| | hydroxy-5-methoxypentyl)piperidin-1-yl)(3- | | | | 1.80-2.14 (m), 2.24 (m), |
| | aminocyclohexyl)methanone | | | | 2.40, 2.56 (m), |
| | | | | | 2.84 (m), 3.04 (m), |
| | | | | | 3.22 (s), 3.24 (t), |
| | | | | | 3.92 (m), 4.20 (m), 4.42, |
| | | | | | 4.84 (m), |
| | | | | | 6.60-6.92 (m), 7.16-7.38 (m), |
| | | | | | 7.70 (d) |
| I-47A | ((1S,3R,4S)-3-amino-4- | 5 | 1.36 | 515 | 0.70-1.00 (m), |
| | hydroxycyclopentyl)((R)-3-((S)-1-(3-fluoro-2- | | | | 1.04-1.60 (m), |
| | phenoxyphenyl)-1-hydroxy-5- | | | | 1.64-2.30 (m), 2.38, 2.56 (m), |
| | methoxypentyl)piperidin-1-yl)methanone | | | | 2.84, 3.04 (m), |
| | | | | | 3.22 (s, t), 3.44 (m), |
| | | | | | 3.80, 4.16 (m), |
| | | | | | 4.22 (m), 4.36, 4.78 (m), |
| | | | | | 6.80 (m), 7.00 (m), |
| | | | | | 7.04-7.32 (m), |
| | | | | | 7.48 (d) |
| I-47B | ((1S,3R,4R)-3-amino-4- | 5 | 1.33 | 515 | 0.76-1.00 (m), |
| | hydroxycyclopentyl)((R)-3-((S)-1-(3-fluoro-2- | | | | 1.10-1.60 (m), |
| | phenoxyphenyl)-1-hydroxy-5- | | | | 1.62-2.36 (m), 2.40, 2.58 (m), |
| | methoxypentyl)piperidin-1-yl)methanone | | | | 2.88, 3.08 (m), |
| | | | | | 3.24 (s, t), 3.40 (m), |
| | | | | | 3.84, 4.16 (m), |
| | | | | | 4.14 (m), 4.38, 4.80 (m), |
| | | | | | 6.80 (m), 7.00 (m), |
| | | | | | 7.18 (m), 7.24 (m), |
| | | | | | 7.56 (d) |
| I-48A | ((R)-3-((S)-1-(3-(4-fluorophenoxy)phenyl)-1- | 5 | 1.44 | 515 | 1.04 (m), |
| | hydroxy-5-methoxypentyl)piperidin-1- | | | | 1.24-2.06 (m), 2.64, 2.92 (dd), |
| | yl)((1S,3R,4S)-3-amino-4- | | | | 3.26 (s), 3.32 (t), |
| | hydroxycyclopentyl)methanone | | | | 3.74 (m), 3.96, |
| | | | | | 4.20 (d), 4.26 (m), 4.46, |
| | | | | | 4.86 (d), 6.82 (m), |
| | | | | | 6.96-7.14 (m), |
| | | | | | 7.34 (m) |
| I-49A | ((R)-3-((S)-1-(2-(3-fluorophenoxy)phenyl)-1- | 5 | 1.36 | 515 | 0.84-2.36 (m), 2.44, |
| | hydroxy-5-methoxypentyl)piperidin-1- | | | | 2.62 (dd), 2.96, |
| | yl)((1S,3R,4S)-3-amino-4- | | | | 3.12 (dd), 3.24 (s), |
| | hydroxycyclopentyl)methanone | | | | 3.30 (t), 3.50 (m), |
| | | | | | 3.92, 4.22 (d), |
| | | | | | 4.24 (m), 4.42, 4.84 (d), |
| | | | | | 6.64 (m), |
| | | | | | 6.76-6.90 (m), 7.16-7.36 (m), |
| | | | | | 7.72 (d) |
| I-50A | ((R)-3-((S)-1-(2-(4-fluorophenoxy)phenyl)-1- | 5 | 1.36 | 515 | 0.86-2.36 (m), 2.46, |
| | hydroxy-5-methoxypentyl)piperidin-1- | | | | 2.62 (dd), 2.96, |
| | yl)((1S,3R,4S)-3-amino-4- | | | | 3.14 (dd), 3.24 (s), |
| | hydroxycyclopentyl)methanone | | | | 3.26 (t), 3.48 (m), |
| | | | | | 3.94 (d), 4.24 (m), |
| | | | | | 4.42 (d), 6.76, |
| | | | | | 6.80 (d), 6.92 (m), |
| | | | | | 7.04-7.24 (m), 7.64 (d) |
| I-51A | ((R)-3-((S)-1-(2-(2-fluorophenoxy)phenyl)-1- | 5 | 1.32 | 515 | 0.96 (m), |
| | hydroxy-5-methoxypentyl)piperidin-1- | | | | 1.14-2.50 (m), 2.64 (dd), 2.98, |
| | yl)((1S,3R,4S)-3-amino-4- | | | | 3.16 (dd), 3.24 (s), |
| | hydroxycyclopentyl)methanone | | | | 3.26 (t), 3.48 (m), |
| | | | | | 3.96 (d), 4.24 (m), |
| | | | | | 4.44 (d), 6.60, |
| | | | | | 6.72 (d), 6.96-7.26 (m), |
| | | | | | 7.64 (d) |
| I-52A | ((1S,3R,4S)-3-amino-4- | 5 | | 515 | 1.05 (brs, 1H), |
| | hydroxycyclopentyl)((R)-3-((S)-1-(2-(2- | | | | 2.95 (m, 1H), 3.27 (s, 3H), |
| | chlorophenyl)phenyl)-1-hydroxy-5- | | | | 3.50 (m, 1H), |
| | methoxypentyl)piperidin-1-yl)methanone | | | | 4.21-4.31 (m, 1H), |
| | | | | | 4.40-4.68 (m, 1H), |
| | | | | | 6.95 (t, 3H), |
| | | | | | 7.14-7.50 (m, 6H), |
| | | | | | 7.61-7.82 (m, 1H) |
| I-53A | ((1S,3R,4S)-3-amino-4- | 5 | | 515 | 1.03 (s, 1H), 3.31 & |
| | hydroxycyclopentyl)((R)-3-((S)-1-(3-chloro-2- | | | | 3.34 (s, 3H), |
| | phenylphenyl)-1-hydroxy-5- | | | | 3.76 (m, 2H), |
| | methoxypentyl)piperidin-1-yl)methanone | | | | 4.44 (m, 2H), |
| | | | | | 7.03-7.18 (m, 3H), |
| | | | | | 7.28-7.42 (m, 5H), |
| | | | | | 8.22 (brs, 2H), |
| I-54A | ((3R,4S)-3-amino-4-hydroxypyrrolidin-1-yl)((R)- | 11 | 1.29 | 516 | 7.51 (dt, 1H), |
| | 3-((S)-1-(3-fluoro-2-phenoxyphenyl)-1-hydroxy- | | | | 7.32-7.27 (m, 2H), |
| | 5-methoxypentyl)piperidin-1-yl)methanone | | | | 7.25-7.21 (m, 1H), |
| | | | | | 7.17-7.12 (m, 1H), 7.03 (t, |
| | | | | | 1H), 6.84 (dd, 2H), |
| | | | | | 4.37 (m, 1H), |
| | | | | | 3.99 (d, 1H), |
| | | | | | 1.83 (td, 1H), |
| | | | | | 1.51 (dt, 1H), |
| | | | | | 1.03 (m, 1H), |
| | | | | | 0.81 (m, 1H) |
| I-55A^{b} | ((1S,3R,4S)-3-amino-4- | 5 | | 517 | 1.03 (s, 1H), |
| | hydroxycyclopentyl)((R)-3-((S)-1-(2- | | | | 3.29 (s, 3H), |
| | phenylphenyl)-1-hydroxy-5- | | | | 3.75 (m, 2H), |
| | methoxypentyl)piperidin-1-yl)methanone | | | | 4.15-4.52 (m, 2H), |
| | | | | | 4.71 (m, 1H), |
| | | | | | 7.39 (m, 4H), |
| | | | | | 7.58 (m, 3H), |
| | | | | | 8.20 (brs, 2H) |
| I-56A | ((1S,3R,4S)-3-amino-4- | 5 | 1.44, | 517 | 7.60-6.87 (m), |
| | hydroxycyclopentyl)((3R)-3-(1-(2- | | 1.49 | | 4.48 (t), 4.28 (m), |
| | (cyclohexylmethoxy)phenyl)-1-hydroxy-5- | | | | 4.21 (m), 4.03-3.93 (m), |
| | methoxypentyl)piperidin-1-yl)methanone | | | | 3.80 (m), 3.31 (m), |
| | | | | | 3.26 (m), 3.24 (m). |
| I-57A | (S)-1-((R)-1-(1-(3-aminopyrrolidin-1-yl)-2- | 13 | 1.32 | 525 | 7.60 (m, 1H), |
| | nitrovinyl)piperidin-3-yl)-5-methoxy-1-(2- | | | | 7.29-7.23 (m, 2H), |
| | phenoxyphenyl)pentan-1-ol | | | | 7.14 (m, 1H), |
| | | | | | 7.08-6.98 (m, 2H), |
| | | | | | 6.89-6.81 (m, 2H), |
| | | | | | 6.76 (m, 1H), 6.38 (s, |
| | | | | | 1H), 3.70 (d, 1H), |
| | | | | | 2.25 (m, 2H), |
| | | | | | 2.00 (m, 1H), |
| | | | | | 0.79 (m, 3H) |
| I-58A | ((R)-3-((S)-1-(2-(o-tolyloxy)phenyl)-1-hydroxy-5- | 16 | 1.44 | 525 | 0.86-2.14 (m), 2.22, |
| | methoxypentyl)piperidin-1-yl)((1S,3R,4R)-3- | | | | 2.24 (s), 2.74 (s), |
| | hydroxy-4- | | | | 3.24 (s), 3.26 (t), |
| | (methylamino)cyclopentyl)methanone | | | | 3.50 (m), 3.92 (d), |
| | | | | | 4.24 (m), 4.44 (d), |
| | | | | | 6.58 (d), 6.72 (d), |
| | | | | | 7.04-7.18 (m), |
| | | | | | 7.26 (m), 7.64 (d) |
| I-58^{b} | ((R)-3-((S)-1-(2-(o-tolyloxy)phenyl)-1-hydroxy-5- | 17 | 1.48 | 525 | 0.84-1.76 (m), |
| | methoxypentyl)piperidin-1-yl)((1S,3S,4R)-3- | | | | 2.24 (s), 2.42 (s), |
| | hydroxy-4- | | | | 2.60 (dd), 2.94 (m), |
| | (methylamino)cyclopentyl)methanone | | | | 3.24 (s), 3.26 (t), 3.94. |
| | | | | | 4.24 (d), 4.12 (m), |
| | | | | | 4.42, 4.86 (d), |
| | | | | | 6.60 (m), 6.70 (d), |
| | | | | | 7.00-7.20 (m), 7.26 (m), |
| | | | | | 7.64 (d) |
| I-58C | ((R)-3-((S)-1-(2-(o-tolyloxy)phenyl)-1-hydroxy-5- | 17 | 1.44 | 525 | 0.82-1.98 (m), |
| | methoxypentyl)piperidin-1-yl)((1R,3S,4R)-3- | | | | 2.22 (s), 2.44 (s), |
| | hydroxy-4- | | | | 2.58 (dd), 3.00 (m), |
| | (methylamino)cyclopentyl)methanone | | | | 3.24 (s), 3.90, 4.26 (d), |
| | | | | | 4.22 (m), 4.44, |
| | | | | | 4.86 (d), 6.52-6.76 (m), |
| | | | | | 7.02-7.20 (m), |
| | | | | | 7.28 (m), 7.64 (d) |
| I-59A | ((R)-3-((S)-1-(2-(2-ethylphenoxy)phenyl)-1- | 5 | 1.52 | 525 | 0.92 (m), 1.18 (t), |
| | hydroxy-5-methoxypentyl)piperidin-1- | | | | 1.22-1.60 (m), |
| | yl)((1S,3R,4S)-3-amino-4- | | | | 1.70 (m), 1.84-2.06 (m), |
| | hydroxycyclopentyl)methanone | | | | 2.64 (q), 2.98, |
| | | | | | 3.16 (dd), 3.24 (s), |
| | | | | | 3.26 (t), 3.46 (m), |
| | | | | | 3.96 (d), 4.26 (m), |
| | | | | | 4.46 (d), 6.56 (d), |
| | | | | | 6.74 (d), 7.04-7.20 (m), |
| | | | | | 7.32 (d), 7.64 (d) |
| I-60A | ((S)-3-(1-(2-(o-tolyloxy)phenyl)-1-hydroxy-6- | 5 | 1.48 | 525 | 7.66 (d), 7.28 (d), |
| | methoxyhexyl)piperidin-1-yl)((1S,3R,4S)-3- | | | | 7.15 (m), 7.06 (m), |
| | amino-4-hydroxycyclopentyl)methanone | | | | 6.71 (d), 6.56 (t), |
| | | | | | 4.90 (m), 4.47 (d), |
| | | | | | 4.24 (m), 3.95 (d), |
| | | | | | 3.49 (d), 3.31 (m), |
| | | | | | 3.26 (m), 3.18 (m), |
| | | | | | 2.97 (t), 2.66 (t), |
| | | | | | 2.26 (s), 2.22 (s), |
| | | | | | 1.92 (m), 1.80 (m), |
| | | | | | 1.46 (m), 1.31 (m), |
| | | | | | 1.21 (m), 0.89 (br |
| | | | | | s). |
| I-61A | ((R)-3-((S)-1-(2-(o-tolyloxy)phenyl)-5-ethoxy-1- | 5 | 1.48 | 525 | 1.08 (t), |
| | hydroxypentyl)piperidin-1-yl)((1S,3R,4S)-3- | | | | 1.10-2.00 (m), 2.22, 2.24 (s), |
| | amino-4-hydroxycyclopentyl)methanone | | | | 2.64 (dd), 2.98, |
| | | | | | 3.16 (dd), 3.34 (t), |
| | | | | | 3.98 (q), 3.48 (m), |
| | | | | | 3.96 (d), 4.24 (m), |
| | | | | | 4.46 (d), 6.56 (d), |
| | | | | | 6.74 (d), |
| | | | | | 7.02-7.20 (m), 7.28 (d), |
| | | | | | 7.66 (d) |
| I-62A | ((R)-3-((S)-1-(2-(o-tolyloxy)phenyl)-1-hydroxy-5- | 7 | 1.44 | 525, | 7.55-7.52 (m, 1H), |
| | methoxypentyl)piperidin-1-yl)((3S,4R)-3-amino- | | | 547 | 7.17-6.87 (m, 5H), |
| | 4-hydroxycyclohexyl)methanone | | | | 6.62-6.42 (m, 2H), |
| | | | | | 4.31-3.58 (m, 3H), |
| | | | | | 3.30-2.76 (m, 6H), |
| | | | | | 2.48-0.77 (m, 23H). |
| I-62B | ((R)-3-((S)-1-(2-(o-tolyloxy)phenyl)-1-hydroxy-5- | 6 | 1.45 | 525, | 7.56-7.52 (m, 1H), |
| | methoxypentyl)piperidin-1-yl)((3R,4S)-3-amino- | | | 547 | 7.17-6.87 (m, 5H), |
| | 4-hydroxycyclohexyl)methanone | | | | 6.63-6.43 (m, 2H), |
| | | | | | 4.32-3.58 (m, 3H), |
| | | | | | 3.30-2.76 (m, 6H), |
| | | | | | 2.48-0.77 (m, 23H). |
| I-63A | ((R)-3-((S)-1-(2-(o-tolyloxy)-3-methylphenyl)-1- | 5 | 1.44 | 525 | 0.40-1.90 (m), |
| | hydroxy-5-methoxypentyl)piperidin-1- | | | | 1.94 (s), 2.38 (s), 3.20, |
| | yl)((1S,3R,4S)-3-amino-4- | | | | 3.24 (s), 3.26 (t), |
| | hydroxycyclopentyl)methanone | | | | 3.50 (m), 3.68, |
| | | | | | 3.98, 4.10 (d), |
| | | | | | 4.24 (m), 4.52, 4.76, |
| | | | | | 4.84 (d), 6.20, |
| | | | | | 6.24 (d), 6.82-7.02 (m), |
| | | | | | 7.12-7.24 (m), |
| | | | | | 7.58 (d) |
| I-64A | ((R)-3-((S)-1-(3-(o-tolyloxy)-2-fluorophenyl)-1- | 5 | 1.48 | 529 | 0.96 (1H, m), |
| | hydroxy-5-methoxypentyl)piperidin-1- | | | | 1.20-1.60 (6H, m), |
| | yl)((1S,3R,4S)-3-amino-4- | | | | 1.65 (1H, dt, 10.3), |
| | hydroxycyclopentyl)methanone | | | | 1.67 (1H, m), 1.82 (1H, |
| | | | | | m), 1.88 (2H, m), |
| | | | | | 2.20 (2H, m), |
| | | | | | 2.29 (3H, m), 2.37 (2H, |
| | | | | | m), 2.61 (1H, t, |
| | | | | | 12.0), 2.62 (3H, s), |
| | | | | | 3.03 (2H, m), |
| | | | | | 3.24 (3H, s), 3.25 (2H, |
| | | | | | m), 3.41 (2H, m), |
| | | | | | 3.52 (1H, m), |
| | | | | | 3.96 (1H, |
| I-65A | ((3R)-3-(1-(2-(4-fluorobenzyloxy)phenyl)-1- | 5 | 1.39, | 529 | 7.62-6.93 (m), |
| | hydroxy-5-methoxypentyl)piperidin-1- | | 1.42 | | 5.06 (m), 4.83 (m), |
| | yl)((1S,3R,4S)-3-amino-4- | | | | 4.44 (m), 4.30-4.21 (m), |
| | hydroxycyclopentyl)methanone | | | | 4.07 (m), 3.93 (m), |
| | | | | | 3.31 (m), 3.23 (m). |
| I-66A | ((R)-3-((S)-1-(2-(o-tolyloxy)-3-fluorophenyl)-1- | 5 | 1.37 | 529 | 0.40-2.28 (m), |
| | hydroxy-5-methoxypentyl)piperidin-1- | | | | 2.40 (four s), 3.18, |
| | yl)((1S,3R,4S)-3-amino-4- | | | | 3.26 (s), 3.72, 4.02, |
| | hydroxycyclopentyl)methanone | | | | 4.16, (d), 4.24 (m), |
| | | | | | 4.30, 4.56, 4.78 (d), |
| | | | | | 6.38, 6.44 (d), |
| | | | | | 6.92 (m), 7.04 (m), |
| | | | | | 7.16 (m), 7.22 (m), |
| | | | | | 7.56 (d) |
| I-67A | ((R)-3-((S)-1-(2-(5-fluoro-2- | 5 | 1.45 | 529 | 0.84-2.00 (m), 2.22, |
| | methylphenoxy)phenyl)-1-hydroxy-5- | | | | 2.24 (s), 2.44, |
| | methoxypentyl)piperidin-1-yl)((1S,3R,4S)-3- | | | | 2.64 (dd), 2.98, |
| | amino-4-hydroxycyclopentyl)methanone | | | | 3.18 (dd), 3.24 (s), |
| | | | | | 3.26 (t), 3.52 (m), |
| | | | | | 3.94 (d), 4.24 (m), |
| | | | | | 4.44 (d), 6.42 (d), |
| | | | | | 6.72 (m), 6.78 (m), |
| | | | | | 7.12-7.30 (m), 7.68 (d) |
| I-68A | ((R)-3-((S)-1-(2-(4-fluoro-2- | 5 | 1.45 | 529 | 0.90-2.00 (m), 2.22, |
| | methylphenoxy)phenyl)-1-hydroxy-5- | | | | 2.24 (s), 2.66 (dd), |
| | methoxypentyl)piperidin-1-yl)((1S,3R,4S)-3- | | | | 2.98, 3.16 (dd), |
| | amino-4-hydroxycyclopentyl)methanone | | | | 3.24 (s), 3.26 (t), |
| | | | | | 3.52 (m), 3.96 (d), |
| | | | | | 4.44 (m), 4.46, |
| | | | | | 4.86 (d), 6.52 (d), |
| | | | | | 6.74 (m), 6.90 (m), |
| | | | | | 7.06 (m), 7.14 (m), |
| | | | | | 7.64 (d) |
| I-69A | ((R)-3-((S)-1-(2-(p-tolyloxy)-3-fluorophenyl)-1- | 5 | 1.37 | 529 | 0.76-2.20 (m), |
| | hydroxy-5-methoxypentyl)piperidin-1- | | | | 2.24 (s), 2.22, 2.58 (dd), |
| | yl)((1S,3R,4S)-3-amino-4- | | | | 2.92, 3.08 (dd), |
| | hydroxycyclopentyl)methanone | | | | 3.22 (st), 3.52 (m), |
| | | | | | 3.84, 4.18 (d), |
| | | | | | 4.26 (m), 4.40, 4.82 (d), |
| | | | | | 6.66 (m), |
| | | | | | 7.04-7.24 (m), 7.72 (d) |
| I-70A | ((R)-3-((S)-1-(2-(o-tolyloxy)-3-fluorophenyl)-1- | 11 | 1.33 | 530 | 7.51 (d, 1H), |
| | hydroxy-5-methoxypentyl)piperidin-1- | | | | 7.26-7.20 (m, 2H), 7.13 (t, |
| | yl)((3R,4S)-3-amino-4-hydroxypyrrolidin-1- | | | | 1H), 7.02 (q, 1H), |
| | yl)methanone | | | | 6.91 (m, 1H), |
| | | | | | 6.42 (m, 1H), |
| | | | | | 4.36 (dq, 1H), |
| | | | | | 2.71 (m, 2H), |
| | | | | | 2.57 (m, 2H), |
| | | | | | 1.19 (m, 1H), |
| | | | | | 0.94 (m, 1H). |
| I-71A | ((R)-3-((S)-1-(2-(2-chlorophenoxy)phenyl)-1- | 5 | 1.4 | 531 | 0.86-2.46 (m), |
| | hydroxy-5-methoxypentyl)piperidin-1- | | | | 2.64 (dd), 2.98, |
| | yl)((1S,3R,4S)-3-amino-4- | | | | 3.16 (dd), 3.24 (s), |
| | hydroxycyclopentyl)methanone | | | | 3.26 (t), 3.40 (m), |
| | | | | | 3.46 (m), 3.94 (d), |
| | | | | | 4.24 (m), 4.44 (d), 6.58, |
| | | | | | 6.72 (d), 6.90 (m), |
| | | | | | 7.08-7.30 (m), |
| | | | | | 7.52 (m), 7.68 (m) |
| I-72A | 3-((R)-3-aminopyrrolidin-1-yl)-4-((R)-3-((S)-1- | 15 | 1.38 | 534 | 0.92 (m, 1H), |
| | hydroxy-5-methoxy-1-(2- | | | | 3.07 (m, 1H), 3.23 (s, |
| | phenoxyphenyl)pentyl)piperidin-1-yl)cyclobut-3- | | | | 3H), 5.75 (br d, 1H), |
| | ene-1,2-dione | | | | 6.83 (d, 1H), |
| | | | | | 6.91 (d, 2H), 7.09 (t, 1H), |
| | | | | | 7.17 (d, 1H), |
| | | | | | 7.22 (m, 1H), 7.36 (t, |
| | | | | | 2H), 7.69 (d, 1H) |
| I-73A | (S)-1-(2-(o-tolyloxy)phenyl)-1-((R)-1-(1-((R)-3- | 13 | 1.35 | 539 | 7.66 (d, 1H), 7.31 (d, |
| | aminopyrrolidin-1-yl)-2-nitrovinyl)piperidin-3-yl)- | | | | 1H), 7.20-7.14 (m, |
| | 5-methoxypentan-1-ol | | | | 2H), 7.11-7.04 (m, |
| | | | | | 2H), 6.75 (d, 1H), |
| | | | | | 6.59 (m, 1H), |
| | | | | | 3.96 (m, 2H), |
| | | | | | 3.85 (m, 1H), |
| | | | | | 3.65 (m, 1H), |
| | | | | | 3.50 (m, 2H), |
| | | | | | 3.10 (m, 1H), 2.27 (s, |
| | | | | | 3H), 2.24 (m, 1H), |
| | | | | | 2.09 (m, 1H), |
| | | | | | 0.99 (m, 2H) |
| I-73B | (S)-1-(2-(o-tolyloxy)phenyl)-1-((R)-1-(1-((S)-3- | 12 | 1.35 | 539 | 7.66 (d, 1H), 7.30 (d, |
| | aminopyrrolidin-1-yl)-2-nitrovinyl)piperidin-3-yl)- | | | | 1H), 7.20-7.14 (m, |
| | 5-methoxypentan-1-ol | | | | 2H), 7.12-7.04 (m, |
| | | | | | 2H), 6.76 (d, 1H), |
| | | | | | 6.53 (m, 1H), |
| | | | | | 4.28 (m, 1H), |
| | | | | | 4.07 (m, 2H), 3.23 (s, |
| | | | | | 3H), 3.22 (m, 1H), |
| | | | | | 2.46 (m, 1H), 2.26 (s, |
| | | | | | 3H), 2.24 (m, 1H), |
| | | | | | 0.98 (m, 1H), |
| | | | | | 0.89 (m, 1H) |
| I-74A | ((R)-3-((R)-1-(2-(o-tolyloxy)-3,5-difluorophenyl)- | 5 | 1.44 | 547 | 1.04-2.10 (m), |
| | 1-hydroxy-5-methoxypentyl)piperidin-1- | | | | 2.26 (s), 2.56, 2.64 (dd), |
| | yl)((1S,3R,4S)-3-amino-4- | | | | 3.26 (s), 3.28 (t), |
| | hydroxycyclopentyl)methanone | | | | 3.80, 4.00 (d), 4.14, |
| | | | | | 4.24 (m), 4.42, |
| | | | | | 4.54 (d), 6.68 (d), |
| | | | | | 6.88-7.04 (m), 7.12 (m, |
| | | | | | 7.24 (m). |
| I-74B^{c} | ((R)-3-((S)-1-(2-(o-tolyloxy)-3,5-difluorophenyl)- | 5 | 1.47 | 547 | 1.06-2.04 (m), |
| | 1-hydroxy-5-methoxypentyl)piperidin-1- | | | | 2.26 (s), 2.48, 2.62 (dd), |
| | yl)((1S,3R,4S)-3-amino-4- | | | | 3.02 (m), 3.26 (s), |
| | hydroxycyclopentyl)methanone | | | | 3.36 (m), 3.52 (m), |
| | | | | | 4.00, 4.22 (d), |
| | | | | | 4.24 (m), 4.54, 4.80 (d), |
| | | | | | 6.64 (m), 6.94 (m), |
| | | | | | 7.02 (m), |
| | | | | | 7.12 (m), 7.24 (m) |
| I-75A | 3-((S)-3-aminopiperidin-1-yl)-4-((R)-3-((S)-1- | 14 | 1.36 | 548 | 0.90 (m, 1H), |
| | hydroxy-5-methoxy-1-(2- | | | | 1.2-1.9 (14H), 2.12 (m, |
| | phenoxyphenyl)pentyl)piperidin-1-yl)cyclobut-3- | | | | 1H), 2.36 (m, 2H), |
| | ene-1,2-dione | | | | 3.04 (m, 1H), |
| | | | | | 3.22 (s, 3H), 3.27 (m, |
| | | | | | 2H), 3.41 (m, 1H), |
| | | | | | 3.50 (m, 1H), |
| | | | | | 3.60 (m, 1H), 3.98 (m, |
| | | | | | 1H), 4.19 (m, 1H), |
| | | | | | 4.43 (m, 1H), |
| | | | | | 6.83 (d, 1H), 6.93 (d, |
| | | | | | 2H), 7.07 (t, 1H), |
| | | | | | 7.17 (m, 1H), |
| | | | | | 7.22 (m, 1H), |
| I-76A | ((S)-3-aminopyrrolidin-1-yl)((R)-3-((S)-1- | 11 | | 480 | 0.94 (m, 1H), |
| | hydroxy-5-methoxy-1-(3′-methylbiphenyl-2- | | | | 2.92 (m, 1H), 2.24 (m, |
| | yl)pentyl)piperidin-1-yl)methanone | | | | 1H), 2.35 (s, 3H), |
| | | | | | 2.72 (m, 2H), |
| | | | | | 3.28 (s, 3H), 3.56 (m, |
| | | | | | 1H), 3.68 (m, 1H), |
| | | | | | 3.82 (m, 1H), |
| | | | | | 6.98 (m, 3H), |
| | | | | | 7.16-7.38 (m, 4H), 7.72 (m, |
| | | | | | 1H) |
| I-77A | ((S)-3-aminopyrrolidin-1-yl)((R)-3-((S)-1- | 11 | | 480 | 0.94 (m, 1H), |
| | hydroxy-5-methoxy-1-(4′-methylbiphenyl-2- | | | | 1.90 (m, 1H), 2.25 (m, |
| | yl)pentyl)piperidin-1-yl)methanone | | | | 1H), 2.38 (s, 3H), |
| | | | | | 2.58 (m, 1H), |
| | | | | | 2.70 (m, 1H), 3.28 (s, |
| | | | | | 3H), 3.44 (m, 1H), |
| | | | | | 3.55 (m, 1H), |
| | | | | | 3.75 (m, 3H), 6.95 (m, |
| | | | | | 1H), 7.00-7.23 (m, |
| | | | | | 5H), 7.30 (m, 1H), |
| | | | | | 7.70 (m, 1H) |
| I-78A | ((1S,3R,4S)-3-ainino-4- | 5 | | 482 | 1.02 (m, 1H), |
| | hydroxycyclopentyl)((R)-3-((S)-1-hydroxy-5- | | | | 2.88 (m, 1H), 3.27 (s, |
| | methoxy-1-(2-(pyridin-2- | | | | 3H), 3.34 (m, 3H), |
| | yl)phenyl)pentyl)piperidin-1-yl)methanone | | | | 3.52 (m, 1H), |
| | | | | | 3.95 (m, 1H), 4.12 (m, |
| | | | | | 1H), 4.26 (m, 1H), |
| | | | | | 4.48&4.74 (m, 1H), |
| | | | | | 7.25 (m, 1H), |
| | | | | | 7.44 (m, 2H), 7.62 (m, |
| | | | | | 1H), 7.95 (m, 2H), |
| | | | | | 8.46 (m, 1H), |
| | | | | | 8.65 (m, 1H) |
| I-79A | ((3R,4S)-3-amino-4-hydroxypyrrolidin-1-yl)((R)- | 11 | | 482 | 0.94 (m, 1H), |
| | 3-((S)-1-(biphenyl-2-yl)-1-hydroxy-5- | | | | 2.66 (m, 2H), 3.28 (s, |
| | methoxypentyl)piperidin-1-yl)methanone | | | | 3H), 4.31 (m, 1H), |
| | | | | | 6.98 (m, 1H), |
| | | | | | 7.23 (m, 3H), 7.35 (m, |
| | | | | | 4H), 7.72 (m, 1H) |
| I-80A | ((3R,4S)-3-amino-4-hydroxypyrrolidin-1-yl)((R)- | 11 | | 483 | 1.00 (m, 1H), |
| | 3-((S)-1-hydroxy-5-methoxy-1-(2-(pyridin-2- | | | | 2.39-2.71 (m, 2H), |
| | yl)phenyl)pentyl)piperidin-1-yl)methanone | | | | 3.26 (s, 3H), 3.93 (m, |
| | | | | | 1H), 4.38 (m, 1H), |
| | | | | | 7.28 (m, 1H), |
| | | | | | 7.45 (m, 2H), 7.61 (m, |
| | | | | | 1H), 7.89 (m, 2H), |
| | | | | | 8.45 (m, 1H), |
| | | | | | 8.65 (m, 1H) |
| I-81A | ((1R,3S)-3-aminocyclopentyl)((R)-3-((S)-1-(4′- | 5 | | 483 | 0.92 (m, 1H), |
| | fluorobiphenyl-2-yl)-1-hydroxy-5- | | | | 2.10 (m, 3H), 2.46 (m, |
| | methoxypentyl)piperidin-1-yl)methanone | | | | 1H), 3.00 (m, 1H), |
| | | | | | 3.28 (s, 3H), |
| | | | | | 3.68 (m, 1H), 4.00 (m, |
| | | | | | 1H)), 4.44&4.62 (m, |
| | | | | | 1H), 6.96 (m, 1H), |
| | | | | | 7.00-7.28 (m, 5H), |
| | | | | | 7.35 (m, 1H), |
| | | | | | 7.75 (m, 1H) |
| I-82A | ((1R,2R)-2-(aminomethyl)cyclopropyl)((RS)-3- | 5 | | 483 | 7.58 (d, 1H), |
| | ((SR)-1-(6-fluoro-3′-methylbiphenyl-2-yl)-1- | | | | 7.38-7.15 (m, 3H), |
| | hydroxy-5-methoxypentyl)piperidin-1- | | | | 7.06-6.96 (m, 2H), |
| | yl)methanone | | | | 6.91 (m, 1H), |
| | | | | | 4.19 (m, 1H), |
| | | | | | 3.07 (m, 1H), |
| | | | | | 2.02 (m, 1H), |
| | | | | | 0.67 (m, 1H). |
| I-83A | ((1R,3S)-3-aminocyclopentyl)((3R)-3-((1S)-1- | 5 | | 483 | 0.92 (m, 1H), |
| | (2′-fluorobiphenyl-2-yl)-1-hydroxy-5- | | | | 2.46 (m, 1H), 3.04 (m, |
| | methoxypentyl)piperidin-1-yl)methanone | | | | 1H), 3.28 (s, 3H), |
| | | | | | 3.68 (m, 1H), |
| | | | | | 4.00 (m, 1H), 4.46& |
| | | | | | 4.62 (m, 1H), 6.98 (m, |
| | | | | | 1H), 7.08-7.28 (m, |
| | | | | | 4H), 7.38-7.44 (m, |
| | | | | | 2H), 7.78 (m, 1H) |
| I-84A | ((1R,3S)-3-aminocyclopentyl)((R)-3-((S)-1-(3′- | 5 | | 483 | 0.94 (m, 1H), |
| | fluorobiphenyl-2-yl)-1-hydroxy-5- | | | | 2.10 (m, 3H), 2.50 (m, |
| | methoxypentyl)piperidin-1-yl)methanone | | | | 1H), 3.00 (m, 1H), |
| | | | | | 3.32 (m, 3H), |
| | | | | | 3.70 (m, 1H), 4.00 (m, |
| | | | | | 1H), 4.45&4.64 (m, |
| | | | | | 1H), 6.80-7.14 (m, |
| | | | | | 4H), 7.25 (m, 1H), |
| | | | | | 7.35 (m, 2H), |
| | | | | | 7.75 (m, 1H) |
| I-85A | ((1R,3S)-3-aminocyclopentyl)((R)-3-((S)-1-(6- | 5 | | 483 | 0.94 (m, 1H), |
| | fluorobiphenyl-2-yl)-1-hydroxy-5- | | | | 2.48 (m, 1H), 3.02 (m, |
| | methoxypentyl)piperidin-1-yl)methanone | | | | 1H), 3.30 (s, 3H), |
| | | | | | 3.66 (m, 1H), |
| | | | | | 3.97 (m, 1H), 4.46& |
| | | | | | 4.56 (m, 1H), |
| | | | | | 7.01-7.27 (m, 3H), 7.37 (m, |
| | | | | | 4H), 7.59 (m, 1H) |
| I-86A | ((S)-3-aminopyrrolidin-1-yl)((3R)-3-((1S)-1-(2′- | 11 | | 484 | 0.93 (m, 1H), |
| | fluorobiphenyl-2-yl)-1-hydroxy-5- | | | | 1.91 (m, 1H), 2.22 (m, |
| | methoxypentyl)piperidin-1-yl)methanone | | | | 1H), 2.62 (m, 2H), |
| | | | | | 3.29 (s, 3H), |
| | | | | | 3.42 (m, 1H), 3.53 (m, |
| | | | | | 1H), 3.70 (m, 1H), |
| | | | | | 3.81 (m, 1H), |
| | | | | | 7.98 (m, 1H), |
| | | | | | 7.06-7.29 (m, 4H), 7.38 (m, |
| | | | | | 2H), 7.64 (m, 1H) |
| I-87A | ((S)-3-aminopyrrolidin-1-yl)((R)-3-((S)-1-(3′- | 11 | | 484 | 0.92 (m, 1H), |
| | fluorobiphenyl-2-yl)-1-hydroxy-5- | | | | 1.92 (m, 1H), 1.96 (m, |
| | methoxypentyl)piperidin-1-yl)methanone | | | | 1H), 2.25 (m, 1H), |
| | | | | | 2.64 (m, 2H), |
| | | | | | 2.78 (m, 3H), 3.44 (m, |
| | | | | | 1H), 3.56 (m, 1H), |
| | | | | | 3.66 (m, 1H), |
| | | | | | 3.80 (m, 1H), |
| | | | | | 6.90-7.12 (m, 4H), 7.25 (m, |
| | | | | | 1H), 7.35 (m, 2H), |
| | | | | | 7.72 (m, 1H) |
| I-88A | ((S)-3-aminopyrrolidin-1-yl)((R)-3-((S)-1-(4′- | 11 | | 484 | 0.93 (m, 1H), |
| | fluorobiphenyl-2-yl)-1-hydroxy-5- | | | | 1.94 (m, 1H), 2.25 (m, |
| | methoxypentyl)piperidin-1-yl)methanone | | | | 1H), 2.75 (m, 2H), |
| | | | | | 3.32 (s, 3H), |
| | | | | | 3.45 (m, 1H), 3.56 (m, |
| | | | | | 1H), 3.68 (m, 1H), |
| | | | | | 3.84 (m, 2H), |
| | | | | | 6.96 (m, 1H), 7.10 (m, |
| | | | | | 2H), 7.22 (m, 3H), |
| | | | | | 7.35 (m, 1H), |
| | | | | | 7.74 (m, 1H) |
| I-89A | ((1S,3R,4S)-3-amino-4- | 5 | | 485 | 2.04 (m, 1H), |
| | hydroxycyclopentyl)((R)-3-((S)-1-hydroxy-5- | | | | 2.18 (m, 1H), 2.31 (s, |
| | methoxy-1-(2-(5-methylfuran-2- | | | | 3H), 2.56 (m, 1H), |
| | yl)phenyl)pentyl)piperidin-1-yl)methanone | | | | 2.94 (m, 1H), |
| | | | | | 3.12 (m, 1H), 3.24 (s, |
| | | | | | 3H), 3.52 (m, 1H), |
| | | | | | 3.94 (m, 1H), |
| | | | | | 4.08 (m, 1H), 4.29 (m, |
| | | | | | 1H), 4.94 & |
| | | | | | 4.76 (m, 1H), 6.08 (m, |
| | | | | | 2H), 7.23 (m, 2H), |
| | | | | | 7.36 (m, 1H), |
| | | | | | 7.76 (m, 1H) |
| I-90A | N-((S)-4-((R)-1-((1S,3R,4S)-3-amino-4- | 5 | | 494 | 1.90 (m, 3H), |
| | hydroxycyclopentanecarbonyl)piperidin-3-yl)-4- | | | | 3.00 (m, 4H), 3.49 (m, |
| | (biphenyl-2-yl)-4-hydroxybutyl)acetamide | | | | 1H), 3.94 (m, 1H), |
| | | | | | 4.22 (m, 1H), |
| | | | | | 4.40&4.56 (m, 1H), |
| | | | | | 6.96 (m, 1H), |
| | | | | | 7.10-7.40 (m, 7H), |
| | | | | | 7.75 (m, 1H) |
| I-91A | N-((S)-4-((R)-1-((3R,4S)-3-amino-4- | 11 | | 495 | 1.05 (m, 1H), |
| | hydroxypyrrolidine-1-carbonyl)piperidin-3-yl)-4- | | | | 1.92 (m, 3H), 2.60 (m, |
| | (biphenyl-2-yl)-4-hydroxybutyl)acetamide | | | | 2H), 3.45 (m, 3H), |
| | | | | | 4.32 (m, 1H), |
| | | | | | 6.96 (m, 1H), |
| | | | | | 7.15-7.42 (m, 7H), 7.74 (m, |
| | | | | | 1H) |
| I-92A | ((1S,3R,4S)-3-amino-4- | 5 | | 495 | 1.02 (m, 1H), |
| | hydroxycyclopentyl)((3R)-3-((1S)-1-hydroxy-5- | | | | 2.01 (s, 3H), 2.42 (m, |
| | methoxy-1-(2′-methylbiphenyl-2- | | | | 1H), 2.95 (m, 2H), |
| | yl)pentyl)piperidin-1-yl)methanone | | | | 3.28 (s, 3H), |
| | | | | | 3.46 (m, 2H), 3.86 (m, |
| | | | | | 1H), 4.22 (m, 1H), |
| | | | | | 4.42 (m, 1H), |
| | | | | | 6.94 (m, 1H), |
| | | | | | 7.10-7.40 (m, 6H), 7.66 (m, |
| | | | | | 1H) |
| I-93A | ((1S,3R,4S)-3-amino-4- | 5 | | 495 | 0.94 (m, 1H), |
| | hydroxycyclopentyl)((R)-3-((S)-1-hydroxy-5- | | | | 2.36 (m, 3H), 2.54 (m, |
| | methoxy-1-(4′-methylbiphenyl-2- | | | | 1H), 3.00 (m, 1H), |
| | yl)pentyl)piperidin-1-yl)methanone | | | | 3.28 (s, 3H), |
| | | | | | 3.46 (m, 1H), 3.95 (m, |
| | | | | | 1H), 4.24 (m, 1H), |
| | | | | | 4.40&4.62 (m, 1H), |
| | | | | | 6.90-7.36 (m, 7H), |
| | | | | | 7.72 (m, 1H) |
| I-94A | ((3R,4S)-3-amino-4-hydroxypyrrolidin-1- | 11 | | 496 | 1.88 (m, 1H), |
| | yl)((3R)-3-((1S)-1-hydroxy-5-methoxy-1-(2′- | | | | 2.03 (s, 3H), 2.58 (m, |
| | methylbiphenyl-2-yl)pentyl)piperidin-1- | | | | 2H), 2.94 (m, 1H), |
| | yl)methanone | | | | 3.28 (s, 3H), |
| | | | | | 3.48 (m, 1H), 3.64 (m, |
| | | | | | 3H), 4.24 (m, 1H), |
| | | | | | 6.92 (m, 1H), |
| | | | | | 6.94-7.32 (m, 6H), |
| | | | | | 7.72 (m, 1H) |
| I-95A | ((3R,4S)-3-amino-4-hydroxypyrrolidin-1-yl)((R)- | 11 | | 496 | 0.93 (m, 1H), |
| | 3-((S)-1-hydroxy-5-methoxy-1-(3′- | | | | 2.36 (s, 3H), 2.65 (m, 2), |
| | methylbiphenyl-2-yl)pentyl)piperidin-1- | | | | 3.09 (m, 1H), |
| | yl)methanone | | | | 3.24 (s, 3H), 4.30 (m, |
| | | | | | 1H), 6.98 (m, 3H), |
| | | | | | 7.20 (m, 3H), |
| | | | | | 7.31 (m, 1H), 7.69 (m, |
| | | | | | 1H) |
| I-96A | ((3R,4S)-3-amino-4-hydroxypyrrolidin-1-yl)((R)- | 11 | | 496 | 0.92 (m, 1H), |
| | 3-((S)-1-hydroxy-5-methoxy-1-(4′- | | | | 2.39 (s, 3H), |
| | methylbiphenyl-2-yl)pentyl)piperidin-1- | | | | 2.50-2.77 (m, 2H), 3.15 (m, |
| | yl)methanone | | | | 1H), 3.25 (s, 3H), |
| | | | | | 4.32 (m, 1H), |
| | | | | | 6.94 (m, 1H), 7.05 (m, |
| | | | | | 2H), 7.19 (m, 3H), |
| | | | | | 7.31 (m, 1H), |
| | | | | | 7.70 (m, 1H) |
| I-97A | ((R)-3-((S)-1-(6-fluoro-3′-methylbiphenyl-2-yl)-1- | 5 | 1.4 | 497 | 2.65 (s, 3H), |
| | hydroxy-5-methoxypentyl)piperidin-1- | | | | 3.27 (s, 3H), 4.18 (m), |
| | yl)((1RS,2RS)-2- | | | | 4.35 (br d), 4.52 (br |
| | ((methylamino)methyl)cyclopropyl)methanone | | | | d), 6.85-7.4 (m), |
| | | | | | 7.59 (d) |
| I-98A | ((1S,3R,4S)-3-amino-4- | 5 | 1.53 | 497 | 7.71-6.97 (m, 7H), |
| | hydroxycyclopentyl)((3S)-3-(1-(6-fluoro-3′- | | | | 4.43-4.16 (m, 3H), |
| | methylbiphenyl-2-yl)-5-methoxypentyl)piperidin- | | | | 3.97-3.79 (m, 1H), |
| | 1-yl)methanone | | | | 3.51-3.41 (m, 2H), |
| | | | | | 3.28 and 3.27 (two |
| | | | | | s, 3H of two |
| | | | | | isomers), 3.26 (m, |
| | | | | | 1H), 2.98-2.86 (m, |
| | | | | | 1H), 2.77-2.42 (m, |
| | | | | | 2H), 2.39 and |
| | | | | | 2.38 (two s, 3H of two |
| | | | | | isomers), |
| | | | | | 2.34-1.94 (m, 6H), |
| | | | | | 1.78-0.90 (m, 9H). |
| I-99A | ((1R,2R)-2-(aminomethyl)cyclopropyl)((RS)-3- | 5 | 1.39 | 499 | 7.51 (d, 1H), |
| | ((SR)-1-(3-fluoro-2-(o-tolyloxy)phenyl)-1- | | | | 7.26-7.19 (m, 2H), |
| | hydroxy-5-methoxypentyl)piperidin-1- | | | | 7.14 (q, 1H), |
| | yl)methanone | | | | 7.00 (m, 1H), |
| | | | | | 6.90 (m, 1H), |
| | | | | | 6.49-6.34 (m, 1H). |
| I-100A | ((1S,3R,4S)-3-amino-4- | 5 | 1.47 | 499 | 7.38-6.96 (m, 7H), |
| | hydroxycyclopentyl)((2S)-2-(1-(6-fluoro-3′- | | | | 4.34-4.18 (m, 2H), |
| | methylbiphenyl-2-yl)-5- | | | | 4.08-3.76 (m, 2H), |
| | methoxypentyl)morpholino)methanone | | | | 3.50-3.34 (m, 4H), |
| | | | | | 3.26 and 3.27 (two |
| | | | | | s, 3H belone to two |
| | | | | | isomers), |
| | | | | | 3.18-3.10 (m, 1H), |
| | | | | | 2.89-2.59 (m, 3H), 2.39 and |
| | | | | | 2.38 (two s, 3H, |
| | | | | | belong to two |
| | | | | | isomers), |
| | | | | | 2.35-1.90 (m, 4H), |
| | | | | | 1.81-1.46 (m, 3H), |
| | | | | | 1.43-1.32 (m 2H), |
| | | | | | 1.18-1.07 (m, 2H) |
| I-101A | ((1S,3R,4S)-3-amino-4- | 5 | | 499 | 0.96 (m, 1H), |
| | hydroxycyclopentyl)((3R)-3-((1S)-1-(2′- | | | | 1.91 (m, 1H), 2.08 (m, |
| | fluorobiphenyl-2-yl)-1-hydroxy-5- | | | | 1H), 2.22 (m, 1H), |
| | methoxypentyl)piperidin-1-yl)methanone | | | | 2.50 (m, 1H), |
| | | | | | 3.01 (m, 1H), 3.28 (s, |
| | | | | | 3H), 3.49 (m, 1H), |
| | | | | | 3.95 (m, 1H), |
| | | | | | 4.23 (m, 1H), 4.42& |
| | | | | | 4.61 (m, 1H), 6.99 (m, |
| | | | | | 1H), 7.10-7.29 (m, |
| | | | | | 4H), 7.39 (m, 2H), |
| | | | | | 7.75 (m, 1H) |
| I-102A | ((1S,3R,4S)-3-amino-4- | 5 | | 499 | 0.93 (m, 1H), |
| | hydroxycyclopentyl)((R)-3-((S)-1-(3′- | | | | 1.92 (m, 1H), 2.20 (m, |
| | fluorobiphenyl-2-yl)-1-hydroxy-5- | | | | 2H), 2.50 (m, 1H), |
| | methoxypentyl)piperidin-1-yl)methanone | | | | 3.00 (m, 1H), |
| | | | | | 3.32 (s, 3H), 3.46 (m, |
| | | | | | 1H), 3.95 (m, 1H), |
| | | | | | 4.25 (m, 1H), |
| | | | | | 4.44&4.62 (m, 1H), |
| | | | | | 6.80-7.18 (m, 4H), |
| | | | | | 7.25 (m, 1H), |
| | | | | | 7.35 (m, 2H), 7.75 (m, |
| | | | | | 1H) |
| I-103A | ((1S,3R,4S)-3-amino-4- | 5 | | 499 | 0.94 (m, 1H), |
| | hydroxycyclopentyl)((R)-3-((S)-1-(4′- | | | | 2.50 (m, 1H), 3.00 (m, |
| | fluorobiphenyl-2-yl)-1-hydroxy-5- | | | | 1H), 3.27 (s, 3H), |
| | methoxypentyl)piperidin-1-yl)methanone | | | | 3.48 (m, 1H), |
| | | | | | 3.95 (m, 1H), 4.25 (m, |
| | | | | | 1H), 4.44&4.62 (m, |
| | | | | | 1H), 6.96 (m, 1H), |
| | | | | | 7.00-7.30 (m, 5H), |
| | | | | | 7.35 (m, 1H), |
| | | | | | 7.74 (m, 1H) |
| I-104A | ((1R,3S)-3-aminocyclopentyl)((3R)-3-((1S)-1- | 5 | | 499 | 1.00 (m, 1H), |
| | (2′-chlorobiphenyl-2-yl)-1-hydroxy-5- | | | | 2.46 (m, 1H), 3.00 (m, |
| | methoxypentyl)piperidin-1-yl)methanone | | | | 1H), 3.27 (s, 3H), |
| | | | | | 3.68 (m, 1H), |
| | | | | | 4.00 (m, 1H), 4.45& |
| | | | | | 4.64 (m, 1H), 6.94 (m, |
| | | | | | 1H), 7.15-7.50 (m, |
| | | | | | 6H), 7.74 (m, 1H) |
| I-105A | ((1R,3S)-3-aminocyclopentyl)((R)-3-((S)-1-(3′- | 5 | | 499 | 0.92 (m, 1H), |
| | chlorobiphenyl-2-yl)-1-hydroxy-5- | | | | 2.12 (m, 1H), 2.50 (m, |
| | methoxypentyl)piperidin-1-yl)methanone | | | | 1H), 3.15 (m, 1H), |
| | | | | | 3.28 (s, 3H), |
| | | | | | 3.70 (m, 1H), 4.02 (m, |
| | | | | | 1H), 4.46&4.61 (m, |
| | | | | | 1H), 6.97 (m, 1H), |
| | | | | | 7.09-7.44 (m, 6H), |
| | | | | | 7.76 (m, 1H) |
| I-106A | ((1R,3S)-3-aminocyclopentyl)((R)-3-((S)-1-(4′- | 5 | | 499 | 0.92 (m, 1H), |
| | chlorobiphenyl-2-yl)-1-hydroxy-5- | | | | 3.05 (m, 1H), 3.28 (s, |
| | methoxypentyl)piperidin-1-yl)methanone | | | | 3H), 3.69 (m, 1H), |
| | | | | | 4.02 (m, 1H), |
| | | | | | 4.46&4.61 (m, 1H), |
| | | | | | 6.96 (m, 1H), |
| | | | | | 7.18-7.39 (m, 6H), |
| | | | | | 7.77 (m, 1H), |
| I-107A | ((3R,4S)-3-amino-4-hydroxypyrrolidin-1-yl)((S)- | 11 | 1.4 | 500 | 7.34-7.28 (m, 3H), |
| | 2-((S)-1-(6-fluoro-3′-methylbiphenyl-2-yl)-5- | | | | 7.21-7.10 (m, 2H), |
| | methoxypentyl)morpholino)methanone | | | | 7.02-9.65 (m 2H), |
| | | | | | 4.36 (m, 1H), |
| | | | | | 3.82-3.87 (m, 1H), |
| | | | | | 3.74-3.40 (m, 12H), 3.28 |
| | | | | | and 3.27 (s, 3H), |
| | | | | | 3.26 (m, 1H), |
| | | | | | 2.95-2.75 (m, 1H), 2.39 |
| | | | | | and 2.37 (s, 3H), |
| | | | | | 1.66 (m, 2H), |
| | | | | | 1.37 (m, 2H), 1.11 (m, 2H) |
| I-108A | ((3R,4S)-3-amino-4-hydroxypyrrolidin-1- | 11 | | 500 | 0.96 (m, 1H), |
| | yl)((3R)-3-((1S)-1-(2′-fluorobiphenyl-2-yl)-1- | | | | 2.62 (m, 2H), 3.08 (m, |
| | hydroxy-5-methoxypentyl)piperidin-1- | | | | 1H), 3.25 (s, 3H), |
| | yl)methanone | | | | 4.29 (m, 1H), |
| | | | | | 6.99 (m, 1H), |
| | | | | | 7.09-7.29 (m, 4H), 7.36 (m, |
| | | | | | 2H), 7.72 (m, 1H) |
| I-109A | ((3R,4S)-3-amino-4-hydroxypyrrolidin-1-yl)((R)- | 11 | | 500 | 0.93 (m, 1H), |
| | 3-((S)-1-(3′-fluorobiphenyl-2-yl)-1-hydroxy-5- | | | | 2.15 (m, 1H), 2.64 (m, |
| | methoxypentyl)piperidin-1-yl)methanone | | | | 2H), 3.11 (m, 1H), |
| | | | | | 3.27 (m, 3H), |
| | | | | | 4.31 (m, 1H), |
| | | | | | 6.89-7.14 (m, 4H), 7.22 (m, |
| | | | | | 1H), 7.43 (m, 2H), |
| | | | | | 7.69 (m, 1H) |
| I-110A | ((3R,4S)-3-amino-4-hydroxypyrrolidin-1-yl)((R)- | 11 | | 500 | 0.95 (m, 1H), |
| | 3-((S)-1-(4′-fluorobiphenyl-2-yl)-1-hydroxy-5- | | | | 2.66 (m, 2H), 3.28 (m, |
| | methoxypentyl)piperidin-1-yl)methanone | | | | 3H), 3.69 (m, 1H), |
| | | | | | 4.32 (m, 1H), |
| | | | | | 6.98 (m, 1H), |
| | | | | | 7.08-7.27 (m, 5H), 7.35 (m, |
| | | | | | 1H), 7.70 (m, 1H) |
| I-111A | ((3R,4S)-3-amino-4-hydroxypyrrolidin-1-yl)((R)- | 11 | | 500 | 0.93 (m, 1H), |
| | 3-((S)-1-(6-fluorobiphenyl-2-yl)-1-hydroxy-5- | | | | 2.62 (m, 2H), 3.14 (m, |
| | methoxypentyl)piperidin-1-yl)methanone | | | | 1H), 3.26 (s, 3H), |
| | | | | | 4.29 (m, 1H), |
| | | | | | 7.05 (m, 1H), 7.19 (m, |
| | | | | | 2H), 7.35 (m, 4H), |
| | | | | | 7.56 (m, 1H) |
| I-112A | ((S)-3-aminopyrrolidin-1-yl)((3R)-3-((1S)-1-(2′- | 11 | | 500 | 1.02 (m, 1H), |
| | chlorobiphenyl-2-yl)-1-hydroxy-5- | | | | 1.80 (m, 2H), 2.25 (m, |
| | methoxypentyl)piperidin-1-yl)methanone | | | | 1H), 3.25 (s, 3H), |
| | | | | | 3.36 (m, 2H), |
| | | | | | 3.65 (m, 2H), 3.84 (m, |
| | | | | | 2H), 6.95 (m, 1H), |
| | | | | | 7.20-7.46 (m, 6H), |
| | | | | | 7.66 (m, 1H) |
| I-113A | ((S)-3-aminopyrrolidin-1-yl)((R)-3-((S)-1-(3′- | 11 | | 500 | 0.91 (m, 1H), |
| | chlorobiphenyl-2-yl)-1-hydroxy-5- | | | | 1.94 (m, 1H), 2.25 (m, |
| | methoxypentyl)piperidin-1-yl)methanone | | | | 1H), 2.61 (m, 1H), |
| | | | | | 3.31 (s, 3H), |
| | | | | | 6.99 (m, 1H), |
| | | | | | 7.09-7.40 (m, 6H), 7.71 (m, |
| | | | | | 1H) |
| I-114A | ((S)-3-aminopyrrolidin-1-yl)((R)-3-((S)-1-(4′- | 11 | | 500 | 0.92 (m, 1H), |
| | chlorobiphenyl-2-yl)-1-hydroxy-5- | | | | 1.97 (m, 1H), 2.28 (m, |
| | methoxypentyl)piperidin-1-yl)methanone | | | | 1H), 2.55-2.76 (m, |
| | | | | | 2H), 3.24 (s, 3H), |
| | | | | | 6.98 (m, 1H), |
| | | | | | 7.15-7.40 (m, 6H), |
| | | | | | 7.72 (m, 1H) |
| I-115A | ((1S,3R,4S)-3-amino-4- | 5 | | 503 | 0.82 (m, 1H), |
| | hydroxycyclopentyl)((R)-3-((S)-1-(2- | | | | 2.65 (m, 1H), 3.98 (m, |
| | cyclohexenyl-3-fluorophenyl)-1-hydroxy-5- | | | | 1H), 4.19-4.40 (m, |
| | methoxypentyl)piperidin-1-yl)methanone | | | | 1H), 5.42&5.53 (m, |
| | | | | | 1H), 6.92 (m, 1H), |
| | | | | | 7.21 (m, 1H), |
| | | | | | 7.36 (m, 1H) |
| I-116A | ((1S,3R,4S)-3-amino-4- | 5 | | 506 | 7.50 (m, 1H), |
| | hydroxycyclopentyl)((R)-3-((S)-1-(3-fluoro-2- | | | | 7.33-7.15 (m, 2H), |
| | (piperidin-1-yl)phenyl)-1-hydroxy-5- | | | | 4.29 (q, 1H), 4.00 (d, |
| | methoxypentyl)piperidin-1-yl)methanone | | | | 1H), 3.23 (s, 3H), |
| | | | | | 2.56 (m, 1H), |
| | | | | | 0.91 (m, 1H). |
| I-117A | N-(2-((R)-((R)-1-((1S,3R,4S)-3-amino-4- | 5 | | 512 | 1.08 (m, 1H), |
| | hydroxycyclopentanecarbonyl)piperidin-3-yl)(6- | | | | 1.92 (m, 3H), 2.36 (s, |
| | fluoro-3′-methylbiphenyl-2- | | | | 3H), 3.20-3.60 (m, |
| | yl)methoxy)ethyl)acetamide | | | | 4H), 4.03 (m, 1H), |
| | | | | | 4.25 (m, 1H), |
| | | | | | 6.98-7.16 (m, 3H), |
| | | | | | 7.23 (m, 1H), 7.35 (m, |
| | | | | | 2H), 7.44 (m, 1H) |
| I-118A | ((3R,4S)-3-amino-4-hydroxypyrrolidin-1-yl)((R)- | 11 | | 512 | 1.05 (m, 1H), |
| | 3-((S)-1-hydroxy-5-methoxy-1-(3-(o- | | | | 2.19 (s, 3H), 2.38 (m, |
| | tolyloxy)phenyl)pentyl)piperidin-1-yl)methanone | | | | 1H), 2.56 (m, 1H), |
| | | | | | 3.27 (s, 3H), |
| | | | | | 3.65 (m, 5H), 4.02 (m, |
| | | | | | 1H), 4.38 (m, 1H), |
| | | | | | 6.74 (m, 1H), |
| | | | | | 6.84 (m, 1H), 6.95 (m, |
| | | | | | 1H), 7.06 (m, 2H), |
| | | | | | 7.15 (m, 1H), |
| | | | | | 7.27 (m, 2H) |
| I-118B^{c} | ((3R,4R)-3-amino-4-hydroxypyrrolidin-1-yl)((R)- | 11 | | 512 | 0.94 (m, 1H), |
| | 3-((R)-1-hydroxy-5-methoxy-1-(2-(o- | | | | 1.79 (m, 2H), 2.19 (m, |
| | tolyloxy)phenyl)pentyl)piperidin-1-yl)methanone | | | | 1H), 2.25 (s, 3H), |
| | | | | | 2.46 (m, 1H), |
| | | | | | 2.66 (m, 2H), 2.92 (m, |
| | | | | | 1H), 3.23 (s, 3H), |
| | | | | | 3.58 (m, 2H), |
| | | | | | 3.72 (m, 1), 4.13 (m, |
| | | | | | 1H), 6.60 (m, 1H), |
| | | | | | 6.79 (m, 1H), |
| | | | | | 7.00-7.20 (m, 4H), |
| | | | | | 7.29 (m, 1H), 7.69 (m, |
| | | | | | 1H) |
| I-118C | ((3R,4R)-3-amino-4-hydroxypyrrolidin-1-yl)((R)- | 11 | | 512 | 0.97 (m, 1H), |
| | 3-((S)-1-hydroxy-5-methoxy-1-(2-(o- | | | | 1.91 (m, 1H), 2.24 (s, |
| | tolyloxy)phenyl)pentyl)piperidin-1-yl)methanone | | | | 3H), 2.44 (m, 2H), |
| | | | | | 3.22 (s, 3H), |
| | | | | | 3.41 (m, 1H), 3.54 (m, |
| | | | | | 1H), 3.70 (m, 3H), |
| | | | | | 4.08 (m, 1H), |
| | | | | | 4.22 (m, 1H), 6.55 (m, |
| | | | | | 1H), 6.76 (m, 1H), |
| | | | | | 7.00-7.20 (m, 4H), |
| | | | | | 7.39 (m, 1H), |
| | | | | | 7.65 (m, 1H) |
| I-119A | (3-amino-3-methylpyrrolidin-1-yl)((R)-3-((S)-1- | 11 | 1.35 | 512 | 7.58 (t, 1H), |
| | (6-fluoro-3′-methylbiphenyl-2-yl)-1-hydroxy-5- | | | | 7.40-7.18 (m, 3H), |
| | methoxypentyl)piperidin-1-yl)methanone | | | | 7.08-6.94 (m, 3H), 3.70 (t, |
| | | | | | 1H), 2.94 (td, 1H), |
| | | | | | 2.36 (s, 3H), 2.03 (m, |
| | | | | | 1H), 0.93 (m, 1H). |
| I-120A | ((1S,3R,4S)-3-amino-4- | 5 | 1.36 | 513 | 7.75 (dt, 1H), |
| | hydroxycyclopentyl)((RS)-2-((RS)-1-hydroxy-5- | | | | 7.28 (d, 1H), |
| | methoxy-1-(2-(o- | | | | 7.16 (m, 2H), 7.06 (t, |
| | tolyloxy)phenyl)pentyl)morpholino)methanone | | | | 2H), 6.74 (m, 1H), |
| | | | | | 6.53 (d, 1H), 3.81 (d, |
| | | | | | 1H), 3.49 (m, 2H), |
| | | | | | 2.21 (s, 3H), 0.92 (m, |
| | | | | | 1H). |
| I-120B | ((1S,3R,4S)-3-amino-4- | 5 | 1.3 | 513 | 7.67 (d, 1H), 7.28 (d, |
| | hydroxycyclopentyl)((R)-2-((R)-1-hydroxy-5- | | | | 1H), 7.19-7.01 (m, |
| | methoxy-1-(2-(o- | | | | 4H), 6.74 (t, 1H), |
| | tolyloxy)phenyl)pentyl)morpholino)methanone | | | | 6.58 (dd, 1H), |
| | | | | | 4.26 (m, 1H), |
| | | | | | 3.49 (m, 1H), 3.23 (s, |
| | | | | | 3H), 2.95 (m, 1H), |
| | | | | | 0.94 (m, 1H). |
| I-121A | ((R)-3-((S)-1-(3-fluoro-2-(o-tolyloxy)phenyl)-1- | 5 | 1.4 | 514 | 3.98 (m, 1H), |
| | hydroxy-5-methoxypentyl)piperidin-1- | | | | 4.23 (m, 1H), 4.71 (m, |
| | yl)((1RS,2RS)-2- | | | | 1H), 6.35-6.50 (m,), |
| | ((methylamino)methyl)cyclopropyl)methanone | | | | 6.5-7.30 (m), |
| | | | | | 7.54 (m) |
| I-122A | ((1S,3R,4S)-3-amino-4- | 5 | | 513 | 0.98 (m, 1H), |
| | hydroxycyclopentyl)((3R)-3-((1S)-1-(2′-fluoro-5′- | | | | 2.33 (s, 3H), 2.52 (m, |
| | methylbiphenyl-2-yl)-1-hydroxy-5- | | | | 1H), 3.27 (s, 3H), |
| | methoxypentyl)piperidin-1-yl)methanone | | | | 3.49 (m, 1H), |
| | | | | | 3.97 (m, 1H), 4.22 (m, |
| | | | | | 1H), 4.42&4.61 (m, |
| | | | | | 1H), 6.87-7.04 (m, |
| | | | | | 3H), 7.21 (m, 2H), |
| | | | | | 7.35 (m, 1H), |
| | | | | | 7.78 (m, 1H) |
| I-123A | ((1S,3R,4S)-3-amino-4- | 5 | | 513 | 0.90 (m, 1H), |
| | hydroxycyclopentyl)((R)-3-((S)-1-(4-fluoro-3′- | | | | 2.32 (s, 3H), 2.60 (m, |
| | methylbiphenyl-2-yl)-1-hydroxy-5- | | | | 1H), 2.98 (m, 1H), |
| | methoxypentyl)piperidin-1-yl)methanone | | | | 3.22 (s, 3H), |
| | | | | | 3.51 (m, 1H), 3.96 (m, |
| | | | | | 2H), 4.29 (m, 1H), |
| | | | | | 7.02-7.19 (m, 2H), |
| | | | | | 7.28-7.52 (m, 4H), |
| | | | | | 7.82 (m, 1H) |
| I-124A | ((1S,3R,4S)-3-amino-4- | 5 | | 513 | 0.89 (m, 1H), |
| | hydroxycyclopentyl)((R)-3-((R)-1-(5-fluoro-3′- | | | | 1.95 (m, 1H), 2.35 (s, |
| | methylbiphenyl-2-yl)-1-hydroxy-5- | | | | 3H), 2.49 (m, 1H), |
| | methoxypentyl)piperidin-1-yl)methanone | | | | 3.29 (s, 3H), |
| | | | | | 3.49 (m, 1H), 3.63 (m, |
| | | | | | 1H), 3.89 (m, 1H), |
| | | | | | 4.26 (m, 1H), |
| | | | | | 4.41&4.55 (m, 1H), |
| | | | | | 6.70 (m, 1H), |
| | | | | | 6.89-7.10 (m, 3H), |
| | | | | | 7.21 (m, 2H), 7.74 (m, |
| | | | | | 1H) |
| I-125A | ((1S,3R,4S)-3-amino-4- | 5 | | 513 | 0.92 (m, 1H), |
| | hydroxycyclopentyl)((R)-3-((S)-1-(5-fluoro-3′- | | | | 2.35 (m, 3H), 3.02 (m, |
| | methylbiphenyl-2-yl)-1-hydroxy-5- | | | | 1H), 3.29 (s, 3H), |
| | methoxypentyl)piperidin-1-yl)methanone | | | | 3.46 (m, 1H), |
| | | | | | 3.92 (m, 1H), 4.24 (m, |
| | | | | | 1H), 4.42&4.56 (m, |
| | | | | | 1H), 6.70 (m, 1H), |
| | | | | | 6.88-7.10 (m, 3H), |
| | | | | | 7.24 (m, 2H), |
| | | | | | 7.75 (m, 1H) |
| I-126A | ((1S,3R,4S)-3-amino-4- | 5 | | 513 | 0.93 (m, 1H), |
| | hydroxycyclopentyl)((R)-3-((S)-1-(6-fluoro-3′- | | | | 2.06 (m, 1H), 2.35 (m, |
| | methylbiphenyl-2-yl)-1-hydroxy-5- | | | | 3H), 2.52 (m, 1H), |
| | methoxypentyl)piperidin-1-yl)methanone | | | | 2.96 (m, 1H), |
| | | | | | 2.78 (s, 3H), 3.46 (m, |
| | | | | | 1H), 3.92 (m, 1H), |
| | | | | | 4.22 (m, 1H), 4.42 |
| | | | | | & 4.56 (m, 1H), |
| | | | | | 6.94 (m, 1H), |
| | | | | | 7.06 (m, 2H), |
| | | | | | 7.18-7.39 (m, 3H), 7.08 (m, |
| | | | | | 1H) |
| I-127A | ((3R,4S)-3-amino-4-hydroxypyrrolidin-1- | 11 | 1.25 | 514 | 7.66 (d, 1H), 7.28 (d, |
| | yl)((RS)-2-((RS)-1-hydroxy-5-methoxy-1-(2-(o- | | | | 1H), 7.14 (td, 2H), |
| | tolyloxy)phenyl)pentyl)morpholino)methanone | | | | 7.05 (m, 2H), |
| | | | | | 6.75 (d, 1H), 6.55 (t, |
| | | | | | 1H), 4.36 (1H), |
| | | | | | 4.20 (dd, 1H), |
| | | | | | 3.85 (dd, 1H), |
| | | | | | 3.22 (s, 3H), 2.26 (s, |
| | | | | | 3H), 0.96 (m, 1H). |
| I-128A | ((3R,4S)-3-amino-4-hydroxypyrrolidin-1- | 11 | | 514 | 0.94 (m, 1H), |
| | yl)((3R)-3-((1S)-1-(2′-fluoro-5′-methylbiphenyl-2- | | | | 2.42 (s, 3H), 2.64 (m, |
| | yl)-1-hydroxy-5-methoxypentyl)piperidin-1- | | | | 2H), 3.10 (m, 1H), |
| | yl)methanone | | | | 3.25 (s, 3H), |
| | | | | | 4.29 (m, 1H), 6.99 (m, |
| | | | | | 3H), 7.17-7.40 (m, |
| | | | | | 3H), 7.73 (m, 1H) |
| I-129A | ((3R,4S)-3-amino-4-hydroxypyrrolidin-1-yl)((R)- | 11 | | 514 | 0.92 (m, 1H), |
| | 3-((S)-1-(6-fluoro-3′-methylbiphenyl-2-yl)-1- | | | | 2.38 (s, 3H), 2.62 (m, |
| | hydroxy-5-methoxypentyl)piperidin-1- | | | | 2H), 3.07 (m, 1H), |
| | yl)methanone | | | | 3.28 (s, 3H), |
| | | | | | 3.46 (m, 4H), 3.68 (m, |
| | | | | | 3H), 4.30 (m, 1H), |
| | | | | | 6.96-7.04 (m, 3H), |
| | | | | | 7.20-7.38 (m, 3H), |
| | | | | | 7.56 (m, 1H) |
| I-129B | ((3R,4R)-3-amino-4-hydroxypyrrolidin-1-yl)((R)- | 11 | 1.36 | 514 | 7.58 (d, 1H), |
| | 3-((S)-1-(6-fluoro-3′-methylbiphenyl-2-yl)-1- | | | | 7.38-7.27 (m, 2H), |
| | hydroxy-5-methoxypentyl)piperidin-1- | | | | 7.22 (d, 1H), |
| | yl)methanone | | | | 7.03 (m, 2H), 6.97 (t, |
| | | | | | 1H), 4.20 (m, 1H), |
| | | | | | 3.53 (m, 1H), 3.28 (s, |
| | | | | | 3H), 3.11 (m, 1H), |
| | | | | | 2.38 (s, 3H), 0.93 (m, |
| | | | | | 1H). |
| I-130A | ((1S,3R,4S)-3-amino-4- | 5 | 1.26 | 515 | 0.92 (m), |
| | hydroxycyclopentyl)((R)-2-((R)-1-(6-fluoro-3′- | | | | 1.26-1.80 (m), 1.96 (m), |
| | methylbiphenyl-2-yl)-1-hydroxy-5- | | | | 2.08 (m), 2.24 (m), 2.38, |
| | methoxypentyl)morpholino)methanone | | | | 2.40 (s), 2.78 (m), |
| | | | | | 3.08-3.38 (m), |
| | | | | | 3.44 (m), 3.78 (m), |
| | | | | | 4.22 (m), 4.30 lm), |
| | | | | | 7.04 (m), 7.20-7.38 (m), |
| | | | | | 7.60 (d) |
| I-131A | ((1S,3R,4S)-3-amino-4- | 5 | | 515 | 0.91 (m, 1H), |
| | hydroxycyclopentyl)((R)-3-((S)-1-(3′- | | | | 3.00 (m, 1H), 3.29 (s, |
| | chlorobiphenyl-2-yl)-1-hydroxy-5- | | | | 3H), 3.49 (m, 1H), |
| | methoxypentyl)piperidin-1-yl)methanone | | | | 3.95 (m, 1H), |
| | | | | | 4.23 (m, 1H), 4.43 & |
| | | | | | 4.60 (m, 1H), |
| | | | | | 6.96 (m, 1H), |
| | | | | | 7.02-7.41 (m, 1H), 7.70 (m, |
| | | | | | 1H) |
| I-132A | ((1S,3R,4S)-3-amino-4- | 5 | | 515 | 0.93 (m, 1H), |
| | hydroxycyclopentyl)((R)-3-((S)-1-(4′- | | | | 2.00 (m, 1H), 2.18 (m, |
| | chlorobiphenyl-2-yl)-1-hydroxy-5- | | | | 1H), 2.29 (m, 1H), |
| | methoxypentyl)piperidin-1-yl)methanone | | | | 2.43 & 2.56 (m, |
| | | | | | 1H), 2.91 & |
| | | | | | 3.05 (m, 1H), 3.29 (s, |
| | | | | | 3H), 3.49 (m, 1H), |
| | | | | | 3.95 (m, 1H), |
| | | | | | 4.26 (m, 1H), 4.44 & |
| | | | | | 4.62 (m, 1H), |
| | | | | | 6.97 (m, 1H), 7.24 (m, |
| | | | | | 3H), 7.46 (m, 3H), |
| | | | | | 7.70 (m, 1H) |
| I-133A | ((3R,4S)-3-amino-4-hydroxypyrrolidin-1-yl)((R)- | 11 | 1.3 | 516 | 7.70 (m, 1H), |
| | 3-((S)-1-(2-(3-fluorophenoxy)phenyl)-1-hydroxy- | | | | 7.34 (q, 1H), |
| | 5-methoxypentyl)piperidin-1-yl)methanone | | | | 7.26 (td, 1H), |
| | | | | | 7.17 (td, 1H), |
| | | | | | 6.91-6.67 (m, 4H), |
| | | | | | 4.03 (d, 1H), |
| | | | | | 2.23 (td, 2H), |
| | | | | | 1.58 (d, 1H), |
| | | | | | 0.88 (m, 1H). |
| I-134A | ((3R,4S)-3-amino-4-hydroxypyrrolidin-1- | 11 | | 516 | 1.03 (m, 1H), |
| | yl)((3R)-3-((1S)-1-(2′-chlorobiphenyl-2-yl)-1- | | | | 1.89 (m, 1H), 2.66 (m, |
| | hydroxy-5-methoxypentyl)piperidin-1- | | | | 2H), 3.16 (m, 1H), |
| | yl)methanone | | | | 3.25 (s, 3H), |
| | | | | | 3.83 (m, 1H), 4.31 (m, |
| | | | | | 1H), 6.95 (m, 1H), |
| | | | | | 7.19-7.48 (m, 6H), |
| | | | | | 7.14 (m, 1H) |
| I-135A | ((3R,4S)-3-amino-4-hydroxypyrrolidin-1-yl)((R)- | 11 | | 516 | 0.92 (m, 1H), |
| | 3-((S)-1-(3′-chlorobiphenyl-2-yl)-1-hydroxy-5- | | | | 2.62 (m, 2H), 3.28 (s, |
| | methoxypentyl)piperidin-1-yl)methanone | | | | 3H), 4.33 (m, 1H), |
| | | | | | 6.99 (m, 1H), |
| | | | | | 7.09-7.41 (m, 6H), |
| | | | | | 7.70 (m, 1H) |
| I-136A | ((3R,4S)-3-amino-4-hydroxypyrrolidin-1-yl)((R)- | 11 | | 516 | 2.66 (m, 2H), |
| | 3-((S)-1-(4′-chlorobiphenyl-2-yl)-1-hydroxy-5- | | | | 3.18 (m, 1H), 3.29 (s, |
| | methoxypentyl)piperidin-1-yl)methanone | | | | 3H), 3.46 (m, 1H), |
| | | | | | 3.60 (m, 2H), |
| | | | | | 3.66 (m, 1H), 3.78 (m, |
| | | | | | 1H), 4.35 (m, 1H), |
| | | | | | 6.98 (m, 1H), |
| | | | | | 7.18-7.35 (m, 6H), |
| | | | | | 7.68 (m, 1H) |
| I-137A | ((1S,3R,4S)-3-amino-4- | 5 | | 517 | 0.95 (m, 1H), |
| | hydroxycyclopentyl)((R)-3-((S)-1-(3′,6- | | | | 2.98 (m, 1H), 3.29 (s, |
| | difluorobiphenyl-2-yl)-1-hydroxy-5- | | | | 3H), 3.49 (m, 1H), |
| | methoxypentyl)piperidin-1-yl)methanone | | | | 3.92 (m, 1H), |
| | | | | | 4.23 (m, 1H), 4.44 & |
| | | | | | 4.56 (m, 1H), |
| | | | | | 6.83-7.19 (m, 4H), |
| | | | | | 7.39 (m, 2H), 7.58 (m, |
| | | | | | 1H) |
| I-138A | ((3R,4S)-3-amino-4-hydroxypyrrolidin-1- | 11 | | 518 | 0.98 (m, 1H), |
| | yl)((3R)-3-((1S)-1-(2′,3′-difluorobiphenyl-2-yl)-1- | | | | 1.95 (m, 1H), 2.68 (m, |
| | hydroxy-5-methoxypentyl)piperidin-1- | | | | 2H), 3.25 (s, 3H), |
| | yl)methanone | | | | 4.11 (m, 1H), |
| | | | | | 4.39 (m, 1H), 7.26 (m, |
| | | | | | 1H), 7.36-7.50 (m, |
| | | | | | 4H), 7.56 (m, 2H) |
| I-139A | ((3R,4S)-3-amino-4-hydroxypyrrolidin-1-yl)((R)- | 11 | | 518 | 0.93 (m, 1H), |
| | 3-((S)-1-(3′,6-difluorobiphenyl-2-yl)-1-hydroxy- | | | | 2.64 (m, 2H), 3.14 (m, |
| | 5-methoxypentyl)piperidin-1-yl)methanone | | | | 1H), 3.28 (s, 3H), |
| | | | | | 3.46-3.78 (m, 6H), |
| | | | | | 4.34 (m, 1H), |
| | | | | | 6.90-7.14 (m, 4H), |
| | | | | | 7.40 (m, 2H), 7.56 (m, |
| | | | | | 1H) |
| I-140A | N-((4S)-4-((R)-1-((1S,3R,4S)-3-amino-4- | 5 | | 526 | 1.75 (m, 2H), |
| | hydroxycyclopentanecarbonyl)piperidin-3-yl)-4- | | | | 1.88 (m, 3H), 2.37 (m, |
| | (2′-fluoro-5′-methylbiphenyl-2-yl)-4- | | | | 3H), 3.05 (m, 4H), |
| | hydroxybutyl)acetamide | | | | 3.46 (m, 1H), |
| | | | | | 3.95 (m, 1H), 4.24 (m, |
| | | | | | 1H), 4.44 & |
| | | | | | 4.60 (m, 1H), 6.90 (m, |
| | | | | | 3H), 7.20 (m, 2H), |
| | | | | | 7.46 (m, 1H), |
| | | | | | 7.76 (m, 1H) |
| I-141A | N-((S)-4-((R)-1-((1S,3R,4S)-3-amino-4- | 5 | | 526 | 0.90 (m, 1H), |
| | hydroxycyclopentanecarbonyl)piperidin-3-yl)-4- | | | | 1.92 (m, 3H), 2.06 (m, |
| | (6-fluoro-3′-methylbiphenyl-2-yl)-4- | | | | 1H), 2.40 (m, H), |
| | hydroxybutyl)acetamide | | | | 3.05 (m, 3H), |
| | | | | | 3.46 (m, 1H), 3.92 (m, |
| | | | | | 1H), 4.24 (m, 1H), |
| | | | | | 4.44 & 4.55 (m, |
| | | | | | 1H), 6.90-7.12 (m, |
| | | | | | 3H), 7.20-7.45 (m, |
| | | | | | 3H), 7.62 (m, 1H) |
| I-142A | (3-amino-3-(hydroxymethyl)pyrrolidin-1-yl)((R)- | 11 | 1.36 | 526 | 7.65 (dd, 1H), |
| | 3-((S)-1-hydroxy-5-methoxy-1-(2-(o- | | | | 7.29 (d, 1H), |
| | tolyloxy)phenyl)pentyl)piperidin-1-yl)methanone | | | | 7.19-7.13 (m, 2H), |
| | | | | | 7.09-7.03 (m, 2H), |
| | | | | | 6.74 (dd, 1H), |
| | | | | | 6.57 (td, 1H), |
| | | | | | 4.10 (d, 1H), 3.71 (d, |
| | | | | | 1H), 3.63 (d, 2H), |
| | | | | | 2.82 (td, 1H), 2.68 (t, |
| | | | | | 1H), 2.24 (s, 3H), |
| | | | | | 1.62 (d, 1H), |
| | | | | | 0.98 (m, 1H). |
| I-143A | ((3R,4S)-3-amino-4-hydroxypyrrolidin-1-yl)((R)- | 11 | 1.37 | 526 | 7.63 (d, 1H), 7.32 (d, |
| | 3-((S)-1-(2-(2-ethylphenoxy)phenyl)-1-hydroxy- | | | | 1H), 7.19-7.02 (m, |
| | 5-methoxypentyl)piperidin-1-yl)methanone | | | | 3H), 6.74 (d, 1H), |
| | | | | | 6.54 (d, 1H), |
| | | | | | 4.33 (m, 1H), |
| | | | | | 4.09 (d, 1H), 3.23 (s, |
| | | | | | 3H), 1.92 (td, 1H), |
| | | | | | 1.19 (t, 3H), 0.98 (m, |
| | | | | | 1H). |
| I-144A | ((3R,4S)-3-amino-4-hydroxypyrrolidin-1-yl)((R)- | 11 | 1.43 | 526 | 7.73 (d, 1H), 7.38 (d, |
| | 3-((S)-5-ethoxy-1-hydroxy-1-(2-(o- | | | | 1H), 7.27-7.12 (m, |
| | tolyloxy)phenyl)pentyl)piperidin-1-yl)methanone | | | | 4H), 6.84 (d, 1H), |
| | | | | | 6.62 (d, 1H), 4.44 (s, |
| | | | | | 1H), 4.18 (d, 1H), |
| | | | | | 2.32 (s, 3H), 2.01 (td, |
| | | | | | 1H), 1.71 (d, 1H), |
| | | | | | 1.20 (t, 3H). |
| I-145A | N-((S)-4-((R)-1-((3R,4S)-3-amino-4- | 11 | | 527 | 1.92 (m, 3H), |
| | hydroxypyrrolidine-1-carbonyl)piperidin-3-yl)-4- | | | | 2.48 (m, 3H), 2.60 (m, |
| | (6-fluoro-3′-methylbiphenyl-2-yl)-4- | | | | 2H), 3.04 (m, 3H), |
| | hydroxybutyl)acetamide | | | | 3.46 (m, 3H), |
| | | | | | 3.65 (m, 3H), 4.30 (m, |
| | | | | | 1H), 6.96-7.10 (m, |
| | | | | | 3H), 7.20-7.40 (m, |
| | | | | | 3H), 7.56 (m, 1H) |
| I-146A | ((1S,3R,4S)-3-amino-4- | 5 | | 527 | 0.92 (m, 1H), |
| | hydroxycyclopentyl)((R)-3-((S)-1-(6-fluoro-3′,5′- | | | | 2.30 (m, 6H), 2.98 (m, |
| | dimethylbiphenyl-2-yl)-1-hydroxy-5- | | | | 1H), 3.28 (m, 3H), |
| | methoxypentyl)piperidin-1-yl)methanone | | | | 3.49 (m, 1H), |
| | | | | | 4.44&4.56 (m, 1H), |
| | | | | | 6.70 (m, 1H), |
| | | | | | 6.80 (m, 1H), |
| | | | | | 6.99-7.10 (m, 2H), 7.33 (m, |
| | | | | | 1H), 7.57 (m, 1H) |
| I-147A | ((R)-3-((S)-1-(6-fluoro-3′-methylbiphenyl-2-yl)-1- | 11 | 1.35 | 528 | 7.57 (d, 1H), |
| | hydroxy-5-methoxypentyl)piperidin-1- | | | | 7.37-7.23 (m, 3H), |
| | yl)((3S,4R)-3-hydroxy-4- | | | | 7.05-6.95 (m, 3H), |
| | (methylamino)pyrrolidin-1-yl)methanone | | | | 4.37 (m, 1H), 3.27 (s, |
| | | | | | 3H), 3.06 (m, 1H), |
| | | | | | 2.72 (s, 3H), 2.36 (s, |
| | | | | | 3H), 0.93 (m, 1H). |
| I-148A | (3-amino-3-methylpyrrolidin-1-yl)((R)-3-((S)-1- | 11 | 1.37 | 528 | 7.52 (d, 1H), |
| | (3-fluoro-2-(o-tolyloxy)phenyl)-1-hydroxy-5- | | | | 7.22 (m, 2H), 7.13 (t, |
| | methoxypentyl)piperidin-1-yl)methanone | | | | 1H), 7.03 (t, 1H), |
| | | | | | 6.92 (t, 1H), 6.42 (m, |
| | | | | | 1H), 3.08 (m, 1H), |
| | | | | | 2.73 (m, 2H), |
| | | | | | 2.53 (m, 2H), |
| | | | | | 2.39 (d, 3H). |
| I-149A | ((3R,4S)-3-amino-4-hydroxypyrrolidin-1-yl)((R)- | 11 | | 528 | 0.90 (m, 1H), |
| | 3-((S)-1-(6-fluoro-3′,5′-dimethylbiphenyl-2-yl)-1- | | | | 2.33 (s, 6H), 2.16 (m, |
| | hydroxy-5-methoxypentyl)piperidin-1- | | | | 2H), 3.16 (m, 1H), |
| | yl)methanone | | | | 3.28 (s, 3H), |
| | | | | | 3.46 (m, 2H), 3.64 (m, |
| | | | | | 4H), 4.34 (m, 1H), |
| | | | | | 6.75 (m, 1H), |
| | | | | | 6.80 (m, 1H), 7.04 (m, |
| | | | | | 2H), 7.32 (m, 1H), |
| | | | | | 7.56 (m, 1H) |
| I-150A | ((1S,3R,4S)-3-amino-4- | 5 | | 529 | 0.89 (m, 1H), |
| | hydroxycyclopentyl)((R)-3-((S)-1-(6-chloro-3′- | | | | 2.34 (m, 3H), 3.02 (m, |
| | methylbiphenyl-2-yl)-1-hydroxy-5- | | | | 2H), 3.28 (m, 3H), |
| | methoxypentyl)piperidin-1-yl)methanone | | | | 3.46 (m, 1H), |
| | | | | | 3.92 (m, 1H), 4.22 (m, |
| | | | | | 1H), 4.48 (m, 1H), |
| | | | | | 6.88 (m, 1H), |
| | | | | | 6.95 (m, 1H), |
| | | | | | 7.18-7.40 (m, 4H), 7.75 (m, |
| | | | | | 1H) |
| I-151A | ((3R,4R)-3-amino-4-hydroxypyrrolidin-1-yl)((R)- | 11 | 1.38 | 530 | 7.52 (d, 1H), |
| | 3-((S)-1-(3-fluoro-2-(o-tolyloxy)phenyl)-1- | | | | 7.22 (m, 2H), 7.13 (t, |
| | hydroxy-5-methoxypentyl)piperidin-1- | | | | 1H), 7.02 (t, 1H), |
| | yl)methanone | | | | 6.92 (t, 1H), |
| | | | | | 6.42 (dd, 1H), |
| | | | | | 3.08 (t, 1H), 2.53 (td, |
| | | | | | 2H), 2.39 (d, 3H). |
| I-152A | ((3R,4S)-3-amino-4-hydroxypyrrolidin-1-yl)((R)- | 11 | | 530 | 0.92 (m, 1H), |
| | 3-((S)-1-(6-chloro-3′-methylbiphenyl-2-yl)-1- | | | | 1.74 (m, 1H), 2.38 (s, |
| | hydroxy-5-methoxypentyl)piperidin-1- | | | | 3H), 2.62 (m, 2H), |
| | yl)methanone | | | | 3.06 (m, 1H), |
| | | | | | 3.28 (s, 3H), |
| | | | | | 3.40-3.72 (m, 6H), 4.31 (m, |
| | | | | | 1H), 6.95 (m, 2H), |
| | | | | | 7.23-7.41 (m, 4H), |
| | | | | | 7.75 (m, 1H) |
| I-153A | ((1S,3R,4S)-3-amino-4- | 5 | | 531 | 0.95 (m, 1H), |
| | hydroxycyclopentyl)((3R)-3-((1S)-1-(2′,6- | | | | 2.36 (m, 3H), 3.00 (m, |
| | difluoro-5′-methylbiphenyl-2-yl)-1-hydroxy-5- | | | | 1H), 3.25 (s, 3H), |
| | methoxypentyl)piperidin-1-yl)methanone | | | | 3.46 (m, 1H), |
| | | | | | 3.95 (m, 1H), 4.24 (m, |
| | | | | | 1H), 4.45 & |
| | | | | | 4.56 (m, 1H), |
| | | | | | 6.90-7.10 (m, 3H), 7.25 (m, |
| | | | | | 1H), 7.38 (m, 1H), |
| | | | | | 7.65 (m, 1H) |
| I-153B^{c} | ((1S,3R,4S)-3-amino-4- | 5 | | 531 | 0.92 (m, 1H), |
| | hydroxycyclopentyl)((3R)-3-((1R)-1-(2′,6- | | | | 2.35 (s, 3H), 2.56 (m, |
| | difluoro-5′-methylbiphenyl-2-yl)-1-hydroxy-5- | | | | 1H), 3.00 (m, 1H), |
| | methoxypentyl)piperidin-1-yl)methanone | | | | 3.25 (s, 3H), |
| | | | | | 3.50 (m, 1H), 3.96 (m, |
| | | | | | 1H), 4.25 (m, 1H), |
| | | | | | 4.46 (m, 1H), |
| | | | | | 7.06 (m, 1H), 7.24 (m, |
| | | | | | 4H), 7.63 (m, 1H), |
| I-154A | ((1S,3R,4S)-3-amino-4-hydroxycyclopentyl)(2- | 5 | 1.32 | 531 | 7.77 (dd, 1H), |
| | ((R)-1-(6-chloro-3′-methylbiphenyl-2-yl)-1- | | | | 7.38 (m, 1H), |
| | hydroxy-5- | | | | 7.33-7.22 (m, 3H), |
| | methoxypentyl)morpholino)methanone | | | | 6.94 (m, 2H), |
| | | | | | 4.27 (m, 2H), 2.38 (s, |
| | | | | | 3H), 1.36 (t, 3H), |
| | | | | | 0.89 (m, 1H). |
| I-155A | ((1S,3R,4S)-3-amino-4- | 5 | 1.35 | 531 | 0.90 (m), |
| | hydroxycyclopentyl)((R)-2-((R)-1-(6-chloro-3′- | | | | 1.26-1.80 (m), 1.96 (m), |
| | methylbiphenyl-2-yl)-1-hydroxy-5- | | | | 2.08 (m), 2.24 (m), 2.38, |
| | methoxypentyl)morpholino)methanone | | | | 2.40 (s), 2.74, |
| | | | | | 2.82 (m), 3.16 (m), |
| | | | | | 3.24 (s), 3.26 (t), |
| | | | | | 3.30 (m), 3.46 (m), |
| | | | | | 3.78 (m), 4.24 (m), |
| | | | | | 4.28 lm), 6.94 (m), |
| | | | | | 7.20-7.40 (m), 7.78 (m) |
| I-156A | ((3R,4S)-3-amino-4-hydroxypyrrolidin-1- | 11 | | 532 | 0.92 (m, 1H), |
| | yl)((3R)-3-((1S)-1-(2′,6-difluoro-5′- | | | | 2.36 (s, 3H), 2.65 (m, |
| | methylbiphenyl-2-yl)-1-hydroxy-5- | | | | 2H), 3.26 (s, 3H), |
| | methoxypentyl)piperidin-1-yl)methanone | | | | 3.65 (m, 4H), |
| | | | | | 4.34 (m, 1H), |
| | | | | | 6.90-7.10 (m, 3H), 7.25 (m, |
| | | | | | 1H), 7.38 (m, 1H), |
| | | | | | 7.60 (m, 1H) |
| I-157A | ((3R,4S)-3-amino-4-hydroxypyrrolidin-1- | 11 | 1.32 | 532 | 7.75 (d, 1H), 7.37 (d, |
| | yl)((RS)-2-((RS)-1-(6-chloro-3′-methylbiphenyl- | | | | 1H), 7.30 (m, 2H), |
| | 2-yl)-1-hydroxy-5- | | | | 7.23 (m, 1H), |
| | methoxypentyl)morpholino)methanone | | | | 6.96 (m, 2H), |
| | | | | | 4.36 (m, 1H), 2.37 (s, |
| | | | | | 3H), 1.59 (m, 1H), |
| | | | | | 0.89 (m, 1H). |
| I-158A | ((1S,3R,4S)-3-amino-4- | 5 | | 533 | 0.92 (m, 1H), |
| | hydroxycyclopentyl)((R)-3-((S)-1-(3′-chloro-6- | | | | 2.98 (m, 1H), 3.27 (s, |
| | fluorobiphenyl-2-yl)-1-hydroxy-5- | | | | 3H), 3.48 (m, 3H), |
| | methoxypentyl)piperidin-1-yl)methanone | | | | 3.94 (m, 1H), |
| | | | | | 4.25 (m, 1H), 4.45& |
| | | | | | 4.56 (m, 1H), |
| | | | | | 7.00-7.25 (m, 3H), |
| | | | | | 7.3-7.44 (m, 3H), 7.58 (m, |
| | | | | | 1H) |
| I-159A | ((1S,3R,4S)-3-amino-4- | 5 | | 533 | 0.91 (m, 1H), |
| | hydroxycyclopentyl)((R)-3-((S)-1-(6-chloro-3′- | | | | 1.89 (m, 1H), 2.09 (m, |
| | fluorobiphenyl-2-yl)-1-hydroxy-5- | | | | 1H), 2.23 (m, 1H), |
| | methoxypentyl)piperidin-1-yl)methanone | | | | 2.99 (m, 1H), |
| | | | | | 3.29 (s, 3H), 3.49 (m, |
| | | | | | 1H), 3.90 (m, 1H), |
| | | | | | 4.22 (m, 1H), 4.43 |
| | | | | | & 4.55 (m, 1H), |
| | | | | | 6.82-7.05 (m, 2H), |
| | | | | | 7.17 (m, 1H), |
| | | | | | 7.30-7.50 (m, 3H), |
| | | | | | 7.68 (m, 1H) |
| I-160A | ((3R,4S)-3-amino-4-hydroxypyrrolidin-1-yl)((R)- | 11 | | 534 | 0.93 (m, 1H), |
| | 3-((S)-1-(2-fluoro-5-(4-fluorophenoxy)phenyl)-1- | | | | 1.66 (m, 1H), 1.88 (m, |
| | hydroxy-5-methoxypentyl)piperidin-1- | | | | 1H), 2.15 (m, 2H), |
| | yl)methanone | | | | 2.66 (m, 2H), |
| | | | | | 3.26 (s, 3H), 3.66 (m, |
| | | | | | 5H), 4.05 (m, 1H), |
| | | | | | 4.39 (m, 1H), |
| | | | | | 6.86 (m, 1H), |
| | | | | | 6.96-7.10 (m, 5H), 7.23 (m, |
| | | | | | 1H) |
| I-161A | ((3R,4S)-3-amino-4-hydroxypyrrolidin-1-yl)((R)- | 11 | | 534 | 0.94 (m, 1H), |
| | 3-((S)-1-(3′-chloro-6-fluorobiphenyl-2-yl)-1- | | | | 2.65 (m, 2H), 3.15 (m, |
| | hydroxy-5-methoxypentyl)piperidin-1- | | | | 2H), 3.27 (s, 3H), |
| | yl)methanone | | | | 3.40-3.80 (m, 6H), |
| | | | | | 4.35 (m, 1H), |
| | | | | | 7.00-7.25 (m, 3H), |
| | | | | | 7.35-7.48 (m, 3H), |
| | | | | | 7.57 (m, 1H) |
| I-162A | ((3R,4S)-3-amino-4-hydroxypyrrolidin-1-yl)((R)- | 11 | | 534 | 0.92 (m, 1H), |
| | 3-((S)-1-(6-chloro-3′-fluorobiphenyl-2-yl)-1- | | | | 2.65 (m, 2H), 3.11 (m, |
| | hydroxy-5-methoxypentyl)piperidin-1- | | | | 1H), 3.29 (s, 3H), |
| | yl)methanone | | | | 3.61 (m, 2H), |
| | | | | | 4.31 (m, 1H0, |
| | | | | | 6.81-7.03 (m, 2H), 7.16 (m, |
| | | | | | 1H), 7.31-7.49 (m, |
| | | | | | 3H), 7.75 (m, 1H) |
| I-163A | methyl (S)-4-((R)-1-((1S,3R,4S)-3-amino-4- | 5 | 1.24 | 542 | 7.45-6.73 (m, 7H), |
| | hydroxycyclopentanecarbonyl)piperidin-3-yl)-4- | | | | 4.41-4.06 (m, 2H), |
| | (6-fluoro-3′-methylbiphenyl-2-yl)-4- | | | | 3.76-2.74 (m, 8H), |
| | hydroxybutylcarbamate | | | | 2.41-0.90 (m, 18H). |
| I-164A | N-((S)-4-((R)-1-((1S,3R,4S)-3-amino-4- | 5 | | 542 | 1.02 (m, 1H), |
| | hydroxycyclopentanecarbonyl)piperidin-3-yl)-4- | | | | 1.92 (m, 3H), |
| | (6-chloro-3′-methylbiphenyl-2-yl)-4- | | | | 2.34-2.42 (m, 3H), 3.00 (m, |
| | hydroxybutyl)acetamide | | | | 3H), 3.48 (m, 1H), |
| | | | | | 3.92 (m, 1H), |
| | | | | | 4.24 (m, 1H), 4.42& |
| | | | | | 4.52 (m, 1H), |
| | | | | | 6.80-7.06 (m, 2H), |
| | | | | | 7.20-7.42 (m, 4H), 7.76 (m, |
| | | | | | 1H) |
| I-165A | methyl (S)-4-((R)-1-((3R,4S)-3-amino-4- | 11 | 1.21 | 543 | 7.45 (d, J = 8 Hz, |
| | hydroxypyrrolidine-1-carbonyl)piperidin-3-yl)-4- | | | | 1H), 7.24-7.08 (m, |
| | (6-fluoro-3′-methylbiphenyl-2-yl)-4- | | | | 3H), 6.96-6.81 (m, |
| | hydroxybutylcarbamate | | | | 3H), 4.25-4.17 (m, |
| | | | | | 1H), 3.58-2.24 (m, |
| | | | | | 17H), 1.58-0.88 (m, |
| | | | | | 9H). |
| I-165B^{c} | methyl (R)-4-((R)-1-((3R,4S)-3-amino-4- | 11 | 1.22 | 543 | 7.51 (d, J = 7.6 Hz, |
| | hydroxypyrrolidine-1-carbonyl)piperidin-3-yl)-4- | | | | 1H), 7.31-7.14 (m, |
| | (6-fluoro-3′-methylbiphenyl-2-yl)-4- | | | | 3H), 7.02-6.87 (m, |
| | hydroxybutylcarbamate | | | | 3H), 4.31-4.23 (m, |
| | | | | | 1H), 3.64-2.31 (m, |
| | | | | | 17H), 1.59-0.98 (m, |
| | | | | | 9H). |
| I-166A | N-((S)-4-((R)-1-((3R,4S)-3-amino-4- | 11 | | 543 | 1.92 (m, 3H), |
| | hydroxypyrrolidine-1-carbonyl)piperidin-3-yl)-4- | | | | 2.39 (m, 3H), 2.64 (m, |
| | (6-chloro-3′-methylbiphenyl-2-yl)-4- | | | | 2H), 3.06 (m, 2H), |
| | hydroxybutyl)acetamide | | | | 3.14 (m, 1H), |
| | | | | | 3.42 (m, 2H), 3.66 (m, |
| | | | | | 4H), 4.32 (m, 1H), |
| | | | | | 6.88-7.06 (m, 2H), |
| | | | | | 7.20-7.42 (m, 4H), |
| | | | | | 7.73 (m, 1H) |
| I-167A | N-((4S)-4-((R)-1-((1S,3R,4S)-3-amino-4- | 5 | | 544 | 1.02 (m, 1H), |
| | hydroxycyclopentanecarbonyl)piperidin-3-yl)-4- | | | | 1.88 (m, 3H), 2.35 (m, |
| | (2′,6-difluoro-5′-methylbiphenyl-2-yl)-4- | | | | 3H), 2.48 (m, 1H), |
| | hydroxybutyl)acetamide | | | | 3.05 (m, 4H), |
| | | | | | 3.46 (m, 1H), 3.95 (m, |
| | | | | | 1H), 4.24 (m, 1H), |
| | | | | | 4.45 & 4.58 (m, |
| | | | | | 1H), 6.88-7.12 (m, |
| | | | | | 3H), 7.18-7.30 (m, |
| | | | | | 1H), 7.35-7.45 (m, |
| | | | | | 1H), 7.62 (m, 1H) |
| I-168A | ((R)-3-((S)-1-(3-fluoro-2-(o-tolyloxy)phenyl)-1- | 11 | 1.36 | 544 | 7.51 (dd, 1H), |
| | hydroxy-5-methoxypentyl)piperidin-1- | | | | 7.24-7.19 (m, 2H), 7.13 (t, |
| | yl)((3S,4R)-3-hydroxy-4- | | | | 1H), 7.01 (q, 1H), |
| | (methylamino)pyrrolidin-1-yl)methanone | | | | 6.91 (q, 1H), |
| | | | | | 6.42 (dd, 1H), |
| | | | | | 4.42 (dt, 1H), 3.08 (t, |
| | | | | | 1H), 2.72 (s, 3H), |
| | | | | | 2.40 (d, 3H), |
| | | | | | 1.53 (m, 1H), |
| | | | | | 0.92 (m, 1H). |
| I-169A | ((1S,3R,4S)-3-amino-4- | 5 | | 549 | 0.95 (m, 1H), |
| | hydroxycyclopentyl)((R)-3-((S)-1-hydroxy-5- | | | | 3.24 (s, 3H), 3.49 (m, |
| | methoxy-1-(3′-(trifluoromethyl)biphenyl-2- | | | | 1H), 3.94 (m, 1H), |
| | yl)pentyl)piperidin-1-yl)methanone | | | | 4.21 (m, 1H), 4.42 |
| | | | | | & 4.59 (m, 1), |
| | | | | | 6.99 (m, 1H), 7.27 (m, |
| | | | | | 1H), 7.35-7.69 (m, |
| | | | | | 1H) |
| I-170A | ((1S,3R,4S)-3-amino-4- | 5 | | 549 | 0.90 (m, 1H), |
| | hydroxycyclopentyl)((R)-3-((S)-1-(3′,6- | | | | 2.49 (m, 1H), 3.01 (m, |
| | dichlorobiphenyl-2-yl)-1-hydroxy-5- | | | | 1H), 3.28 (s, 3H), |
| | methoxypentyl)piperidin-1-yl)methanone | | | | 3.95 (m, 1H), |
| | | | | | 4.24 (m, 1H), 4.45& |
| | | | | | 4.55 (m, 1H), |
| | | | | | 7.05-7.20 (m, 2H), |
| | | | | | 7.30-7.46 (m, 4H), 7.74 (m, |
| | | | | | 1H) |
| I-171A | ((3R,4S)-3-amino-4-hydroxypyrrolidin-1-yl)((R)- | 11 | | 550 | 0.94 (m, 1H), |
| | 3-((S)-1-hydroxy-5-methoxy-1-(3′- | | | | 2.61 (m, 1H), 3.27 (s, |
| | (trifluoromethyl)biphenyl-2-yl)pentyl)piperidin-1- | | | | 3H), 4.31 (m, 1H), |
| | yl)methanone | | | | 7.00 (m, 1H), |
| | | | | | 7.27 (m, 1H), |
| | | | | | 7.36-7.50 (m, 3H), 7.55 (m, |
| | | | | | 1H), 7.69 (m, 2H) |
| I-172A | ((3R,4S)-3-amino-4-hydroxypyrrolidin-1-yl)((R)- | 11 | | 550 | 0.90 (m, 1H), |
| | 3-((S)-1-(3′,6-dichlorobiphenyl-2-yl)-1-hydroxy- | | | | 2.62 (m, 2H), 3.13 (m, |
| | 5-methoxypentyl)piperidin-1-yl)methanone | | | | 1H), 3.27 (s, 3H), |
| | | | | | 4.34 (m, 1H), |
| | | | | | 7.02-7.19 (m, 2H), |
| | | | | | 7.30-7.46 (m, 4H), |
| | | | | | 7.74 (m, 1H) |
| I-173A | ((1S,3R,4S)-3-amino-4- | 5 | | 551 | 0.92 (m, 1H), |
| | hydroxycyclopentyl)((3R)-3-((1R)-1-(3′-chloro- | | | | 2.49 (m, 1H), 3.26 (s, |
| | 2′,6-difluorobiphenyl-2-yl)-1-hydroxy-5- | | | | 3H), 3.49 (m, 1H), |
| | methoxypentyl)piperidin-1-yl)methanone | | | | 3.96 (m, 1H), |
| | | | | | 4.23 (m, 1H), 4.44& |
| | | | | | 4.62 (m, 1H), |
| | | | | | 7.05-7.26 (3H), |
| | | | | | 7.39-7.61 (m, 3H) |
| I-174A | ((1S,3R,4S)-3-amino-4- | 5 | | 585 | 0.95 (m, 1H), |
| | hydroxycyclopentyl)((R)-2-((R)-1-(6-fluoro-3′- | | | | 2.76 (m, 1H), 3.16 (m, |
| | (trifluoromethoxy)biphenyl-2-yl)-1-hydroxy-5- | | | | 3H), 3.25 (s, 3H), |
| | methoxypentyl)morpholino)methanone | | | | 3.49 (m, 1H), |
| | | | | | 3.81 (m, 2H), 4.30 (m, |
| | | | | | 2H), 7.08 (m, 2H), |
| | | | | | 7.22 (m, 1H), |
| | | | | | 7.35 (m, 2H), 7.52 (m, |
| | | | | | 2H) |
| I-175A | methyl (S)-4-((R)-1-((1S,3R,4S)-3-amino-4- | 5 | | 558 | 1.04 (m, 1H), |
| | hydroxycyclopentanecarbonyl)piperidin-3-yl)-4- | | | | 2.35 (m, 3H), 2.94 (m, |
| | (6-chloro-3′-methylbiphenyl-2-yl)-4- | | | | 3H), 3.44 (m, 1H), |
| | hydroxybutylcarbamate | | | | 3.62 (m, 3H), |
| | | | | | 3.90 (m, 1H), 4.25& |
| | | | | | 4.56 (m, 1H), |
| | | | | | 6.80-7.05 (m, 2H), |
| | | | | | 7.20-7.45 (m, 4H), 7.75 (m, |
| | | | | | 1H) |
| I-176A | methyl (S)-4-((R)-1-((3R,4S)-3-amino-4- | 11 | | 559 | 1.02 (m, 1H), |
| | hydroxypyrrolidine-1-carbonyl)piperidin-3-yl)-4- | | | | 2.38 (m, 3H), 2.65 (m, |
| | (6-chloro-3′-methylbiphenyl-2-yl)-4- | | | | 2H), 3.46 (m, 2H), |
| | hydroxybutylcarbamate | | | | 3.65 (m, 6H), |
| | | | | | 4.34 (m, 1H), |
| | | | | | 6.86-7.04 (m, 2H), |
| | | | | | 7.20-7.42 (m, 4H), 7.75 (m, |
| | | | | | 1H) |
| I-177A | methyl 2-((R)-((R)-1-((1S,3R,4S)-3-amino-4- | 5 | | 585 | 0.95 (m, 1H), |
| | hydroxycyclopentanecarbonyl)piperidin-3-yl)(6- | | | | 2.76 (m, 1H), 3.16 (m, |
| | fluoro-3′-methylbiphenyl-2- | | | | 3H), 3.25 (s, 3H), |
| | yl)methoxy)ethylcarbamate | | | | 3.49 (m, 1H), |
| | | | | | 3.81 (m, 2H), 4.30 (m, |
| | | | | | 2H), 7.08 (m, 2H), |
| | | | | | 7.22 (m, 1H), |
| | | | | | 7.35 (m, 2H), 7.52 (m, |
| | | | | | 2H) |
| I-178A | ((3R,4S)-3-amino-4-hydroxypyrrolidin-1-yl)(3- | 19 | | 491 | 0.90 (m, 2H), |
| | ((R)-1-(6-fluoro-3′-methylbiphenyl-2-yl)-5- | | | | 1.45 (m, 2H), 1.61 (m, |
| | methoxypentyl)phenyl)methanone | | | | 2H), 2.01 (m, 3H), |
| | | | | | 3.25 (s, 3H), 3.63 (s, |
| | | | | | 3H), 3.95 (m, 2H), |
| | | | | | 4.18 (m, 2H), |
| | | | | | 6.65 (m, 2H), |
| | | | | | 6.96-7.41 (m, 9H) |
| I-178B | ((3R,4S)-3-amino-4-hydroxypyrrolidin-1-yl)(3- | 19 | | 491 | 0.85 (m, 2H), |
| | ((S)-1-(6-fluoro-3′-methylbiphenyl-2-yl)-5- | | | | 1.39 (m, 3H), 1.52 (m, |
| | methoxypentyl)phenyl)methanone | | | | 2H), 1.95 (m, 3H), |
| | | | | | 3.18 (s, 3H), 3.57 (s, |
| | | | | | 3H), 3.87 (m, 2H), |
| | | | | | 4.14 (m, 2H), |
| | | | | | 6.58 (m, 2H), |
| | | | | | 6.89-7.32 (m, 9H) |
| I-179A | ((1S,3R,4S)-3-amino-4- | 5 | | 499 | 1.09 (m, 4H), |
| | hydroxycyclopentyl)((R)-3-((S)-(6-fluoro-3′- | | | | 1.93 (m, 1H), 2.15 (m, |
| | methylbiphenyl-2-yl)(3- | | | | 2H), 2.34 (s, 3H), |
| | methoxypropoxy)methyl)piperidin-1- | | | | 2.61 (m, 2H), |
| | yl)methanone | | | | 2.86 (m, 2H), 3.20 (s, |
| | | | | | 3H), 3.40 (m, 3H), |
| | | | | | 3.75 (m, 1H), |
| | | | | | 4.00 (m, 1H), 4.21 (m, |
| | | | | | 1H), 6.98 (m, 2H), |
| | | | | | 7.07 (m, 1H), |
| | | | | | 7.19 (m, 1H), 7.25 (m, |
| | | | | | 2H), 7.39 (m, 1H) |
| I-180A | ((1S,3R,4S)-3-amino-4- | 5 | | 501 | 1.67 (m, 2H), |
| | hydroxycyclopentyl)((R)-2-((S)-(6-fluoro-3′- | | | | 1.77 (m, 1H), 2.04 (m, |
| | methylbiphenyl-2-yl)(3- | | | | 1H), 2.21 (m, 2H), |
| | methoxypropoxy)methyl)morpholino)methanone | | | | 2.37 (s, 3H), 2.60 (m |
| | | | | | 1H), 3.24 (s, 3H), |
| | | | | | 3.50 (m, 1H), |
| | | | | | 3.80 (m, 2H), 4.24 (m, |
| | | | | | 2H), 7.07 (m, 3H), |
| | | | | | 7.19 (m, 1H), |
| | | | | | 7.35 (m, 3H) |
| I-181A | ((3R,4S)-3-amino-4-hydroxypyrrolidin-1-yl)(3-(1- | 2 | | | 1.02-1.59 (m, 4H), |
| | (6-fluoro-3′-methylbiphenyl-2-yl)-1-hydroxy-5- | | | | 2.13 (m, 5H), |
| | methoxypentyl)phenyl)methanone | | | | 3.25 (s, 3H), 3.55 (m, |
| | | | | | 2H), 3.74 (m, 1H), |
| | | | | | 4.15 (m, 1H), |
| | | | | | 4.57 (s, 3H), 6.15 (m, |
| | | | | | 1H), 6.58 (m, 1H), |
| | | | | | 7.06 (m, 6H), |
| | | | | | 7.31 (m, 1H), 7.42 (m, |
| | | | | | 1H), 7.85 (m, 1H) |
| I-182A | N-((R)-4-((S)-1-((1S,3R,4S)-3-amino-4- | 5 | | 510 | 1.08 (m, 1H), |
| | hydroxycyclopentanecarbonyl)piperidin-3-yl)-4- | | | | 1.28 (m, 3H), 1.67 (m, |
| | (6-fluoro-3′-methylbiphenyl-2-yl)butyl)acetamide | | | | 6H), 1.89 (s, 3H), |
| | | | | | 2.38 (s, 3H), |
| | | | | | 2.55 (m, 2H), 3.01 (m, |
| | | | | | 2H), 3.50 (m, 1H), |
| | | | | | 3.89 (m, 1H), |
| | | | | | 4.25 (m, 1H), 4.45 (m, |
| | | | | | 1H), 7.00 (m, 3H), |
| | | | | | 7.17 (m, 2H), |
| | | | | | 7.34 (m, 2H) |
| I-183A | ((R)-3-((S)-1-(6-fluoro-3′-methylbiphenyl-2-yl)-1- | 5 | 1.47 | 511 | 0.97 (d), 1.03 (d), |
| | hydroxy-5-methoxypentyl)piperidin-1- | | | | 2.29 (s), 2.32 (s), |
| | yl)((1RS,2RS,3RS)-2-methyl-3- | | | | 2.66 (s), 3.28 (s), |
| | ((methylamino)methyl)cyclopropyl)methanone | | | | 4.11 (br d), 4.60 (t), |
| | | | | | 6.8-7.6 |
| I-184A | ((R)-3-((S)-1-(6-fluoro-3′-methylbiphenyl-2-yl)-1- | 5 | 1.42 | 511 | 0.86 (d), 2.37 (s), |
| | hydroxy-5-methoxypentyl)piperidin-1- | | | | 2.85 (s), 2.87 (s), |
| | yl)((1RS,2RS)-1-methyl-2- | | | | 3.15 (s), 6.5-7.60 |
| | ((methylamino)methyl)cyclopropyl)methanone | | | | |
| I-185A | ((R)-3-((S)-1-(6-fluoro-3′-methylbiphenyl-2-yl)-1- | 5 | 1.42 | 511 | 1.07 (s), 2.35 (s), |
| | hydroxy-5-methoxypentyl)piperidin-1- | | | | 2.67 (s), 2.72 (s), |
| | yl)((1RS,2RS)-2-methyl-2- | | | | 3.29 (s), 4.02 (m), |
| | ((methylamino)methyl)cyclopropyl)methanone | | | | 4.3-4.6, 6.8-7.6 |
| I-186A | N-(2-((S)-((R)-4-((1S,3R,4S)-3-amino-4- | 5 | | 514 | 1.75 (m, 1H), |
| | hydroxycyclopentanecarbonyl)morpholin-2- | | | | 1.89 (s, 3H), 2.03 (m, |
| | yl)(6-fluoro-3′-methylbiphenyl-2- | | | | 1H), 2.25 (m, 2H), |
| | yl)methoxy)ethyl)acetamide | | | | 2.39 (s, 3H), |
| | | | | | 2.70 (m, 1H), 3.20 (m, |
| | | | | | 4H), 3.50 (m, 2H), |
| | | | | | 3.80 (m, 2H), |
| | | | | | 4.35 (m, 3H), 7.11 (m, |
| | | | | | 4H), 7.35 (m, 3H) |
| I-187A | ((3R,4S)-3-amino-4-hydroxypyrrolidin-1-yl)((R)- | 11 | | 516 | 0.93 (m, 1H), |
| | 2-((R)-1-(6-fluoro-3′-methylbiphenyl-2-yl)-1- | | | | 1.41 (m, 4H), 1.62 (m, |
| | hydroxy-5- | | | | 1H), 2.38 (s, 3H), |
| | methoxypentyl)morpholino)methanone | | | | 2.91 (m, 2H), |
| | | | | | 3.18 (m, 1H), 3.28 (s, |
| | | | | | 3H), 3.52 (m, 4H), |
| | | | | | 3.75 (m, 2H), |
| | | | | | 4.34 (m, 1H), 7.02 (m, |
| | | | | | 3H), 7.22 (m, 1H), |
| | | | | | 7.31 (m, 2H), |
| | | | | | 7.57 (m, 1H) |
| I-188A | 6-((S)-1-((R)-1-((1S,3R,4S)-3-amino-4- | 20 | | 520 | 7.99 (d, J = 0.84 Hz, |
| | hydroxycyclopentanecarbonyl)piperidin-3-yl)-1- | | | | 1H), 7.70 (t, J = 7.4 Hz, |
| | hydroxy-5-methoxypentyl)-3′-methylbiphenyl-3- | | | | 1H), |
| | carbonitrile | | | | 7.33-7.22 (m, 3H), |
| | | | | | 7.06-6.95 (m, 2H), 4.57 and |
| | | | | | 4.42 (m, 1H), 4.31 |
| | | | | | and 4.24 (m, 1H), |
| | | | | | 3.93 (m, 1H), |
| | | | | | 4.50 (m, 1H), 3.35 and |
| | | | | | 3.34 (s, 3H), 3.30 |
| | | | | | and 3.16 (m, 1H), |
| | | | | | 3.28 (m 2H), 3.04 |
| | | | | | and 2.90 (m, 1H), |
| | | | | | 2.55-1.18 (m, 18H), |
| | | | | | 0.85 (m 1H) |
| I-189A | methyl (R)-4-((S)-1-((1S,3R,4S)-3-amino-4- | 5 | | 526 | 1.06 (m, 1H), |
| | hydroxycyclopentanecarbonyl)piperidin-3-yl)-4- | | | | 1.25 (m, 3H), 1.97 (m, |
| | (6-fluoro-3′-methylbiphenyl-2-yl)butylcarbamate | | | | 1H), 2.37 (s, 3H), |
| | | | | | 2.50 (m, 2H), |
| | | | | | 2.97 (m, 3H), 3.49 (m, |
| | | | | | 1H), 3.60 (s, 3H), |
| | | | | | 3.85 (m, 1H), |
| | | | | | 4.24 (m, 1H), 4.45 (m, |
| | | | | | 1H), 6.98 (m, 3H), |
| | | | | | 7.17 (m, 2H), |
| | | | | | 7.34 (m, 2H) |
| I-189B | methyl (S)-4-((S)-1-((1S,3R,4S)-3-amino-4- | 5 | | 526 | 1.02 (m, 1H), |
| | hydroxycyclopentanecarbonyl)piperidin-3-yl)-4- | | | | 1.74 (m, 3H), 2.00 (m, |
| | (6-fluoro-3′-methylbiphenyl-2-yl)butylcarbamate | | | | 4H), 2.22 (m, 1H), |
| | | | | | 2.37 (s, 3H), |
| | | | | | 2.50 (m, 2H), 2.94 (m, |
| | | | | | 3H), 3.43 (m, 2H), |
| | | | | | 3.59 (s, 3H), |
| | | | | | 4.20 (m, 2H), 7.00 (m, |
| | | | | | 3H), 7.20 (m, 2H), |
| | | | | | 7.39 (m, 2H) |
| I-190A | ((1S,3R,4S)-3-amino-4- | 5 | | 527 | 1.00 (m, 1H), |
| | hydroxycyclopentyl)((R)-2-((R)-1-hydroxy-5- | | | | 1.43 (m, 3H), 1.76 (m, |
| | methoxy-1-(3-methoxy-3′-methylbiphenyl-2- | | | | 2H), 2.31 (s, 3H), |
| | yl)pentyl)morpholino)methanone | | | | 3.17 (m, 1H), |
| | | | | | 3.29 (d, 3H), 3.49 (m, |
| | | | | | 3H), 3.90 (d, 3H), |
| | | | | | 3.95 (m, 1H), |
| | | | | | 4.06 (m, 1H), 4.21 (m, |
| | | | | | 2H), 6.59 (m, 1H), |
| | | | | | 7.01 (m, 5H), |
| | | | | | 7.22 (m, 1H) |
| I-191A | ((1S,3R,4S)-3-amino-4- | 5 | | 529 | 0.90 (m, 3H), |
| | hydroxycyclopentyl)((R)-2-((R)-1-(3′-ethyl-6- | | | | 1.24 (m, 3H), 2.05 (m, |
| | fluorobiphenyl-2-yl)-1-hydroxy-5- | | | | 4H), 2.27 (m, 1H), |
| | methoxypentyl)morpholino)methanone | | | | 2.67 (m, 2H), |
| | | | | | 3.15 (m, 3H), 3.30 (s, |
| | | | | | 3H), 3.48 (m, 2H), |
| | | | | | 3.75 (m, 3H), |
| | | | | | 4.23 (m, 2H), 7.03 (m, |
| | | | | | 3H), 7.28 (m, 3H), |
| | | | | | 7.59 (m, 1H) |
| I-192A | methyl 2-((S)-((R)-4-((1S,3R,4S)-3-amino-4- | 5 | | 530 | 1.78 (m, 1H), |
| | hydroxycyclopentanecarbonyl)morpholin-2- | | | | 2.04 (m, 1H), 2.21 (m, |
| | yl)(6-fluoro-3′-methylbiphenyl-2- | | | | 2H), 2.39 (s, 3H), |
| | yl)methoxy)ethylcarbamate | | | | 2.72 (m, 1H), |
| | | | | | 3.14 (m, 3H), 3.49 (m, |
| | | | | | 2H), 3.59 (s, 3H), |
| | | | | | 3.79 (m, 1H), |
| | | | | | 4.25 (m, 2H), 4.51 (m, |
| | | | | | 1H), 7.10 (m, 3H), |
| | | | | | 7.22 (m, 1H), |
| | | | | | 7.36 (m, 3H) |
| I-193A | ((1S,3R,4S)-3-amino-4- | 5 | | 531 | 0.84 (m, 1H), |
| | hydroxycyclopentyl)((R)-2-((R)-1-(6-fluoro-3′- | | | | 1.35 (m, 4H), 1.63 (m, |
| | methoxybiphenyl-2-yl)-1-hydroxy-5- | | | | 1H), 3.14 (s, 3H), |
| | methoxypentyl)morpholino)methanone | | | | 3.19 (s, 3H), |
| | | | | | 3.52 (m, 2H), 3.74 (m, |
| | | | | | 4H), 4.19 (m, 1H), |
| | | | | | 6.71 (m, 2H), |
| | | | | | 6.95 (m, 2H), 7.27 (m, |
| | | | | | 2H), 7.52 (m, 1H) |
| I-193B | ((1S,3R,4S)-3-amino-4- | 5 | | 531 | 0.92 (m, 1H), |
| | hydroxycyclopentyl)((R)-2-((S)-1-(6-fluoro-3′- | | | | 1.48 (m, 4H), 1.71 (m, |
| | methoxybiphenyl-2-yl)-1-hydroxy-5- | | | | 1H), 2.00 (m, 2H), |
| | methoxypentyl)morpholino)methanone | | | | 3.26 (s, 3H), 3.27 (s, |
| | | | | | 3H), 3.49 (m, 1H), |
| | | | | | 3.59 (s, 1H), |
| | | | | | 3.81 (m, 3H), 4.23 (m, |
| | | | | | 1H), 6.78 (m, 2H), |
| | | | | | 7.00 (m, 2H), |
| | | | | | 7.33 (m, 2H), 7.59 (m, |
| | | | | | 1H) |
| I-194A | ((1S,3R,4S)-3-amino-4- | 5 | | 535 | 0.91 (m, 1H), |
| | hydroxycyclopentyl)((R)-2-((R)-1-(3′-chloro-6- | | | | 2.76 (m, 2H), 3.05 (m, |
| | fluorobiphenyl-2-yl)-1-hydroxy-5- | | | | 1H), 3.28 (s, 3H), |
| | methoxypentyl)morpholino)methanone | | | | 3.49 (m, 1H), |
| | | | | | 3.63 (m, 1H), 3.81 (m, |
| | | | | | 2H), 4.30 (m, 2H), |
| | | | | | 7.06 (m, 1H), |
| | | | | | 7.19 (m, 2H), 7.40 (m, |
| | | | | | 3H), 7.60 (m, 1H) |
| I-195A | ((1S,3R,4S)-3-amino-4- | 5 | | 541 | 0.71 (m, 2H), |
| | hydroxycyclopentyl)((R)-2-((R)-1-(3′- | | | | 0.95 (m, 4H), 2.75 (m, |
| | cyclopropyl-6-fluorobiphenyl-2-yl)-1-hydroxy-5- | | | | 10H), 3.28 (s, 3H), |
| | methoxypentyl)morpholino)methanone | | | | 3.49 (m, 4H), |
| | | | | | 3.75 (m, 2H), 4.25 (m, |
| | | | | | 2H), 6.86-7.15 (m, |
| | | | | | 5H), 7.30 (m, 2H), |
| | | | | | 7.59 (m, 1H) |
| I-196A | ((1S,3R,4S)-3-amino-4- | 5 | | 545 | 0.91 (m, 2H), |
| | hydroxycyclopentyl)((R)-2-((R)-1-(6-chloro-3′- | | | | 1.25 (m, 3H), 2.01 (m, |
| | ethylbiphenyl-2-yl)-1-hydroxy-5- | | | | 3H), 2.26 (m, 1H), |
| | methoxypentyl)morpholino)methanone | | | | 2.67 (m, 2H), |
| | | | | | 3.26 (s, 3H), 3.49 (m, |
| | | | | | 1H), 3.26 (m, 2H), |
| | | | | | 4.24 (m, 2H), |
| | | | | | 6.97 (m, 2H), 7.30 (m, |
| | | | | | 4H), 7.74 (m, 1H) |
| I-197A | ((1S,3R,4S)-3-amino-4- | 5 | | 545 | 0.90 (m, 1H), |
| | hydroxycyclopentyl)((R)-2-((R)-1-(6-chloro-3′,4′- | | | | 2.33 (m, 6H), 2.78 (m, |
| | dimethylbiphenyl-2-yl)-1-hydroxy-5- | | | | 2H), 3.17 (m, 3H), |
| | methoxypentyl)morpholino)methanone | | | | 3.48 (m, 2H), |
| | | | | | 4.25 (m, 2H), 6.89 (m, |
| | | | | | 2H), 7.25 (m, 3H), |
| | | | | | 7.76 (m, 1H) |
| I-198A | ((1S,3R,4S)-3-amino-4- | 5 | | 545 | 0.92 (m, 1H), 1.39 (t, |
| | hydroxycyclopentyl)((R)-2-((R)-1-(3′-ethoxy-6- | | | | 3H), 2.76 (m, 1H), |
| | fluorobiphenyl-2-yl)-1-hydroxy-5- | | | | 3.16 (m, 2H), |
| | methoxypentyl)morpholino)methanone | | | | 3.27 (s, 3H), 3.48 (m, |
| | | | | | 1H), 3.76 (m, 2H), |
| | | | | | 4.06 (m, 2H), |
| | | | | | 4.29 (m, 2H), 6.75 (m, |
| | | | | | 2H), 7.00 (m, 2H), |
| | | | | | 7.32 (m, 2H), |
| | | | | | 7.59 (m, 1H) |
| I-199A | ((1S,3R,4S)-3-amino-4- | 5 | | 545 | 0.96 (m, 1H), |
| | hydroxycyclopentyl)((R)-2-((R)-1-(6-fluoro-3- | | | | 1.31 (m, 2H), 1.48 (m, |
| | methoxy-3′-methylbiphenyl-2-yl)-1-hydroxy-5- | | | | 2H), 1.80 (m, 1H), |
| | methoxypentyl)morpholino)methanone | | | | 2.35 (s, 3H), |
| | | | | | 2.46 (m, 1H), 2.70 (m, |
| | | | | | 1H), 3.27 (d, 3H), |
| | | | | | 3.51 (m, 3H), |
| | | | | | 3.85 (d, 3H), 4.31 (m, |
| | | | | | 1H), 4.49 (d, 2H), |
| | | | | | 7.00 (m, 4H), |
| | | | | | 7.13 (m, 1H), 7.23 (m, |
| | | | | | 1H) |
| I-200A | ((1S,3R,4S)-amino-4- | 5 | | 547 | 0.89 (m, 1H), |
| | hydroxycyclopentyl)((R)-2-((R)-1-(6-chloro-3′- | | | | 1.40 (m, 6H), 1.68 (m, |
| | methoxybiphenyl-2-yl)-1-hydroxy-5- | | | | 2H), 2.75 (m, 2H), |
| | methoxypentyl)morpholino)methanone | | | | 3.26 (s, 3H), |
| | | | | | 3.49 (m, 2H), 3.79 (s, |
| | | | | | 3H), 4.25 (m, 2H), |
| | | | | | 6.70 (m, 2H), |
| | | | | | 6.99 (m, 1H), 7.33 (m, |
| | | | | | 3H), 7.75 (m, 1H) |
| I-201A | ((1S,3R,4S)-3-amino-4- | 5 | | 547 | 0.91 (m, 1H), |
| | hydroxycyclopentyl)((R)-2-((R)-1-(6-fluoro-3′- | | | | 1.50 (m, 6H), 2.23 (m, |
| | (methylthio)biphenyl-2-yl)-1-hydroxy-5- | | | | 2H), 2.50 (m, 3H), |
| | methoxypentyl)morpholino)methanone | | | | 2.70 (m, 1H), |
| | | | | | 3.17 (m, 2H), 3.27 (s, |
| | | | | | 3H), 3.28 (s, 3H), |
| | | | | | 3.49 (m, 1H), |
| | | | | | 3.59 (s, 1H), 3.78 (m, |
| | | | | | 2H), 4.29 (m, 1H), |
| | | | | | 7.01 (m, 3H), |
| | | | | | 7.34 (m, 3H), 7.59 (m, |
| | | | | | 1H) |
| I-201B | ((1S,3R,4S)-3-amino-4- | 5 | | 547 | 0.91 (m, 1H), |
| | hydroxycyclopentyl)((R)-2-((S)-1-(6-fluoro-3′- | | | | 1.49 (m, 5H), 2.50 (m, |
| | (methylthio)biphenyl-2-yl)-1-hydroxy-5- | | | | 2H), 2.78 (m, 1H), |
| | methoxypentyl)morpholino)methanone | | | | 3.15 (m, 1H), |
| | | | | | 3.28 (s, 3H), 3.29 (s, 3H), |
| | | | | | 3.48 (m, 1H), |
| | | | | | 3.59 (s, 1H), 3.80 (m 2H), |
| | | | | | 4.30 (m, 1H), |
| | | | | | 7.02 (m, 3H), 7.34 (m, |
| | | | | | 3H), 7.58 (m, 1H) |
| I-202A | ((1S,3R,4S)-3-amino-4- | 5 | | 551 | 0.99 (m, 1H), |
| | hydroxycyclopentyl)((2R)-2-(1-(3′,6- | | | | 1.28 (m, 1H), 2.26 (m, |
| | dichlorobiphenyl-2-yl)-1-hydroxy-5- | | | | 2H), 2.85 (m, 1H), |
| | methoxypentyl)morpholino)methanone | | | | 3.40 (d, 3H), 3.64 (m, |
| | | | | | 2H), 3.90 (m, 2H), |
| | | | | | 4.41 (m, 2H), |
| | | | | | 7.22 (m, 2H), 7.50 (m, |
| | | | | | 4H), 7.86 (m, 1H) |
| I-203A | ((1S,3R,4S)-3-amino-4- | 5 | | 559 | 0.91 (m, 1H), 1.27 (t, |
| | hydroxycyclopentyl)((R)-2-((R)-1-(6-chloro-3′- | | | | 6H), 2.77 (m, 1H), |
| | isopropylbiphenyl-2-yl)-1-hydroxy-5- | | | | 2.94 (m, 1H), |
| | methoxypentyl)morpholino)methanone | | | | 3.27 (s, 3H), 3.73 (m, |
| | | | | | 2H), 4.24 (m, 2H), |
| | | | | | 6.96 (m, 2H), |
| | | | | | 7.32 (m, 4H), 7.76 (m, |
| | | | | | 1H) |
| I-204A | ((1S,3R,4S)-3-amino-4- | 5 | | 563 | 0.88 (m, 1H), |
| | hydroxycyclopentyl)((R)-2-((R)-1-(6-chloro-3′- | | | | 2.02 (m, 2H), 2.23 (m, |
| | (methylthio)biphenyl-2-yl)-1-hydroxy-5- | | | | 1H), 2.46 (d, 3H), |
| | methoxypentyl)morpholino)methanone | | | | 2.75 (m, 1H), |
| | | | | | 3.14 (m, 2H), 3.26 (d, |
| | | | | | 2H), 3.47 (m, 1H), |
| | | | | | 3.61 (m, 1H), |
| | | | | | 3.77 (m, 2H), 4.25 (m, |
| | | | | | 2H), 6.90 (m, 1H), |
| | | | | | 6.98 (m, 1H), |
| | | | | | 7.33 (m, 4H), 7.75 (m, |
| | | | | | 1H) |
| I-205A | ((1S,3R,4S)-3-amino-4- | 5 | | 569 | 0.98 (m, 1H), |
| | hydroxycyclopentyl)((R)-2-((R)-1-(6-fluoro-3′- | | | | 2.73 (m, 2H), 3.13 (m, |
| | (trifluoromethyl)biphenyl-2-yl)-1-hydroxy-5- | | | | 3H), 3.23 (s, 3H), |
| | methoxypentyl)morpholino)methanone | | | | 3.49 (m, 1H), |
| | | | | | 3.80 (m, 2H), 4.30 (m, |
| | | | | | 2H), 7.06 (m, 1H), |
| | | | | | 7.52 (m, 6H) |
| I-205B | ((1S,3R,4S)-3-amino-4- | 5 | | 569 | 0.98 (m, 1H), |
| | hydroxycyclopentyl)((R)-2-((S)-1-(6-fluoro-3′- | | | | 2.74 (m, 2H), 3.10 (m, |
| | (trifluoromethyl)biphenyl-2-yl)-1-hydroxy-5- | | | | 3H), 3.27 (s, 3H), |
| | methoxypentyl)morpholino)methanone | | | | 3.48 (m, 1H), |
| | | | | | 3.81 (m, 2H), 4.30 (m, |
| | | | | | 2H), 7.08 (m, 1H), |
| | | | | | 7.36-7.72 (m, 6H) |
| I-206A | ((1S,3R,4S)-3-amino-4- | 5 | 1.53 | 517 | 0.01 (s, 4.5H), |
| | hydroxycyclopentyl)((R)-3-((S)-1-hydroxy-5- | | | | 0.02 (s, 4.5H), 2.84 (s, |
| | methoxy-1-(2-(trimethylsilyl)benzofuran-7- | | | | 1.5H), 2.86 (s, |
| | yl)pentyl)piperidin-1-yl)methanone | | | | 1.5H), 3.20 (m, 1H), |
| | | | | | 3.60 (br d, 0.5H), |
| | | | | | 3.5 (m, 1.5H), 4.12 (br |
| | | | | | d, 0.5H), 4.65 (br d, |
| | | | | | 0.5H), 6.69 (d, 1H), |
| | | | | | 6.84 (m, 1H), |
| | | | | | 7.08 (m, 1H), 7.16 (m, |
| | | | | | 1H) |
| I-207A | ((1S,3R,4S)-3-amino-4-hydroxycyclopentyl)(3- | 5 | 1.62 | 533 | 0.02 (9H, s), |
| | (1-hydroxy-5-methoxy-1-(2- | | | | 3.58 (m, 1H), 4.10 (m, |
| | (trimethylsilyl)benzo[b]thiophen-4- | | | | 1H), 4.60 (m, 1H), |
| | yl)pentyl)piperidin-1-yl)methanone | | | | 6.9-7.5 (4H) |
| I-208A^{b} | ((1S,3R,4S)-3-amino-4- | 5 | | 517 | 0.80-1.99 (m, 22H), |
| | hydroxycyclopentyl)((R)-3-((S)-1-hydroxy-5- | | | | 2.25 (m, 5H), |
| | methoxy-1-(spiro[benzo[d][1,3]dioxole-2,1′- | | | | 2.87 (m, 1H), 3.26 (d, |
| | cyclohexane]-4-yl)pentyl)piperidin-1- | | | | 3H), 3.31 (m, 3H), |
| | yl)methanone | | | | 3.72 (m, 1H), |
| | | | | | 4.10 (m, 3H), 4.39 (m, |
| | | | | | 1H), 4.77 (m, 1H), |
| | | | | | 6.75 (m, 3H), |
| | | | | | 8.14 (brs, 3H) |
| I-209A^{b} | ((1S,3R,4S)-3-amino-4- | 5 | | 531 | 0.89 (m, 1H), |
| | hydroxycyclopentyl)((R)-3-((S)-1,5-dimethoxy-1- | | | | 1.06 (m, 1H), 2.13 (m, |
| | (spiro[benzo[d][1,3]dioxole-2,1′-cyclohexane]-4- | | | | 4H), 2.49 (m, 10H), |
| | yl)pentyl)piperidin-1-yl)methanone | | | | 3.18 (m, 3H), |
| | | | | | 3.31 (s, 3H), 3.33 (s, 3H), |
| | | | | | 3.72 (m, 1H), |
| | | | | | 4.25 (m, 1H), 4.43 (m, |
| | | | | | 1H), 6.74 (m, 3H), |
| | | | | | 8.18 (brs, 2H) |
| I-210A^{b} | ((1S,3R,4S)-3-amino-4- | 5 | | 517 | 1.07 (m, 2H), |
| | hydroxycyclopentyl)((R)-3-((S)-1,5-dimethoxy-1- | | | | 1.45-1.90 (m, 11H), |
| | (spiro[benzo[d][1,3]dioxole-2,1′-cyclopentane]- | | | | 1.91-2.48 (m, 9H), |
| | 4-yl)pentyl)piperidin-1-yl)methanone | | | | 3.22 (s, 3H), 3.25 (s, 3H), |
| | | | | | 3.70 (m, 2H), |
| | | | | | 4.42 (m, 1H), |
| | | | | | 6.61-6.90 (m, 3H), 8.17 (brs, |
| | | | | | 2H) |
| I-211A | ((1S,3R,4S)-3-amino-4- | 5 | 1.22 | 502 | 1.47 (s), 1.48 (s), |
| | hydroxycyclopentyl)((3R)-3-(1-(2-tert- | | | | 3.2 (3.19 (s), |
| | butylbenzo[d]oxazol-7-yl)-1-hydroxy-5- | | | | 3.21 (s), 3.52 (m), |
| | methoxypentyl)piperidin-1-yl)methanone | | | | 3.95 (m), 4.47 (m), |
| | | | | | 7.25 (m), 7.45-7.6 (m) |
| I-212A | ((1S,3R,4S)-3-amino-4- | 5 | 1.35 | 473 | 7.34 (t, 1H), 7.14 (m, |
| | hydroxycyclopentyl)((R)-3-((S)-1-(2- | | | | 2H), 6.44 (d, 1H), |
| | ethylbenzofuran-7-yl)-1-hydroxy-5- | | | | 3.21 (s, 3H), 2.78 (m, |
| | methoxypentyl)piperidin-1-yl)methanone | | | | 2H), 1.30 (m, 3H). |
| I-213A | ((1S,3R,4S)-3-amino-4- | 5 | 1.43 | 501 | 0.98 (m, 6H), |
| | hydroxycyclopenlyl)((R)-3-((S)-1-hydroxy-1-(2- | | | | 3.20 (s), 3.22 (s), |
| | isobutylbenzofuran-7-yl)-5- | | | | 3.54 (m), 3.92 (m), |
| | methoxypentyl)piperidin-1-yl)methanone | | | | 4.46 (m), 4.95 (m), |
| | | | | | 6.44 (d, 1H), 7.16 (m, |
| | | | | | 1H), 7.34 (m, 1H), |
| | | | | | 7.40 (m, 1H) |
| I-214A | ((1S,3R,4S)-3-amino-4- | 5 | 1.52 | 517 | 0.02 (s, 9H), |
| | hydroxycyclopentyl)((R)-3-((S)-1-hydroxy-5- | | | | 2.86 (s), 2.91 (s), |
| | methoxy-1-(2-(trimethylsilyl)benzofuran-4- | | | | 3.60 (m), 4.10 (m), |
| | yl)pentyl)piperidin-1-yl)methanone | | | | 4.60 (m), 6.8-7.1 (4H) |
| I-215A | ((1S,3R,4S)-3-amino-4- | 5 | | 501 | 0.89 (m, 1H), |
| | hydroxycyclopentyl)((R)-3-((S)-1-(2-tert- | | | | 1.29 (m, 4H), 1.37 (d, |
| | butylbenzofuran-7-yl)-1-hydroxy-5- | | | | 9H), 1.49 (m, 4H), |
| | methoxypentyl)piperidin-1-yl)methanone | | | | 1.70 (m, 3H), |
| | | | | | 2.03 (m, 2H), 2.23 (m, |
| | | | | | 3H), 2.48 (m, 3H), |
| | | | | | 2.92 (m, 1H), |
| | | | | | 3.20 (s, 3H), 3.55 (m, |
| | | | | | 1H), 4.30 (m, 1H), |
| | | | | | 6.42 (m, 1H), |
| | | | | | 7.14 (m, 1H), 7.37 (m, |
| | | | | | 2H) |
| I-216A | ((3R,4S)-3-amino-4-hydroxypyrrolidin-1-yl)((R)- | 11 | | 502 | 0.89 (m, 1H), |
| | 3-((S)-1-(2-tert-bulylbenzofuran-7-yl)-1-hydroxy- | | | | 1.26 (m, 4H), 1.38 (s, |
| | 5-methoxypentyl)piperidin-1-yl)methanone | | | | 9H), 1.45 (m, 3H), |
| | | | | | 1.60 (m, 1H), |
| | | | | | 2.00 (m, 1H), 2.30 (m, |
| | | | | | 1H), 2.55 (m, 3H), |
| | | | | | 3.19 (s, 3H), |
| | | | | | 3.24 (m, 2H), 3.47 (m, |
| | | | | | 1H), 3.64 (m, 5H), |
| | | | | | 4.12 (m, 1H), |
| | | | | | 4.37 (m, 1H), 6.42 (s, |
| | | | | | 1H), 7.14 (m, 1H), |
| | | | | | 7.34 (m, 1H), |
| | | | | | 7.39 (m, 1H) |
| I-257A | ((1S,3R,4S)-3-amino-4-hydroxycyclopentyl)(2- | 26 | | 567 | 0.8-1.0 (m, 1H), |
| | (1-(6-chloro-3′-ethylbiphenyl-2-yl)-1-hydroxy-5- | | | | 1.20-1.40 (m, 6H), |
| | methoxypentyl)morpholino)methanone | | | | 1.40-1.55 (m, 3H), |
| | | | | | 1.60-1.80 (m, 1H), |
| | | | | | 1.90-2.15 (m, 2H), |
| | | | | | 2.20-2.35 (m, 1H), |
| | | | | | 2.60-2.80 (m, 3H), |
| | | | | | 3.10-3.25 (m, 2H), |
| | | | | | 3.25-3.35 (m, 5H), |
| | | | | | 3.50 (m, 1H), |
| | | | | | 3.65-3.90 (m, 2H), |
| | | | | | 4.20-4.37 (m, 2H), |
| | | | | | 6.90-7.00 (m, 2H), |
| | | | | | 7.20-7.42 (m, 4H), |
| | | | | | 7.80 (m, 1H). |

^{a1}H NMR spectra were acquired in CD₃OD unless otherwise indicated.
^{b1}H NMR spectrum acquired in CDCl₃.
^{c}Minor isomer separated by chromatography.

The following are compounds of the invention:

| | | Synthetic Method | | | |
|---|---|---|---|---|---|
| Cpd. No. | Name | Example No. | LC_MS Method | t_{R} (min) | Mass observed |
| I-217A | methyl 4-((R)-1-((1R,3S)-3- | 5 | 3 | 1.76 | 495.3 |
| | aminocyclopentanecarbonyl)piperidin-3-yl)-4- | | | | |
| | hydroxy-4-(2-(pyridin-4- | | | | |
| | yl)phenyl)butylcarbamate | | | | |
| I-218A | 2-((R)-((R)-4-((1R,3S)-3- | 5 | 3 | 2.22 | 498.2 |
| | aminocyclopentanecarbonyl)morpholin-2-yl)(6- | | | | |
| | fluoro-3′-methylbiphenyl-2-yl)methoxy)-N- | | | | |
| | ethylacetamide | | | | |
| I-219A | N-(4-((R)-1-((1R,3S)-3- | 5 | 3 | 2.11 | 508.3 |
| | aminocyclopentanecarbonyl)piperidin-3-yl)-4- | | | | |
| | hydroxy-4-(2-(o-tolyloxy)phenyl)butyl)acetamide | | | | |
| I-219B^{a} | N-(4-((R)-1-((1R,3S)-3- | 5 | 3 | 2.24 | 508.3 |
| | aminocyclopentanecarbonyl)piperidin-3-yl)-4- | | | | |
| | hydroxy-4-(2-(o-tolyloxy)phenyl)butyl)acetamide | | | | |
| I-220A | ((1R,3S)-3-aminocyclopentyl)((R)-3-((S)-1-(2- | 5 | | | 509.2 |
| | (2,6-dimethylphenoxy)phenyl)-1-hydroxy-5- | | | | |
| | methoxypentyl)piperidin-1-yl)methanone | | | | |
| I-221A | methyl 4-(1-((1S,3R,4S)-3-amino-4- | 26 | 3 | 1.72 | 511.2 |
| | hydroxycyclopentanecarbonyl)piperidin-3-yl)-4- | | | | |
| | hydroxy-4-(2-(pyridin-3- | | | | |
| | yl)phenyl)butylcarbamate | | | | |
| I-222A | ((1S,3R,4S)-3-amino-4- | 25 | 3 | 2.52 | 513.2 |
| | hydroxycyclopentyl)((R)-2-((R)-1-(6-chloro-3′- | | | | |
| | ethylbiphenyl-2-yl)-1-hydroxypent-4- | | | | |
| | enyl)morpholino)methanone | | | | |
| I-223A | methyl 2-((4-((1R,3S)-3- | 5 | 3 | 2.31 | 514.2 |
| | aminocyclopentanecarbonyl)morpholin-2-yl)(6- | | | | |
| | fluoro-3′-methylbiphenyl-2- | | | | |
| | yl)methoxy)ethylcarbamate | | | | |
| I-224A | 2-((S)-((R)-4-((1S,3R,4S)-3-amino-4- | 26 | 3 | 2.18 | 514.2 |
| | hydroxycyclopentanecarbonyl)morpholin-2- | | | | |
| | yl)(6-fluoro-3′-methylbiphenyl-2-yl)methoxy)-N- | | | | |
| | ethylacetamide | | | | |
| I-225A | ((1R,3S)-3-aminocyclopentyl)((R)-2-((R)-1-(4′,6- | 5 | 3 | 2.26 | 517.2 |
| | difluoro-3′-methylbiphenyl-2-yl)-1-hydroxy-5- | | | | |
| | methoxypentyl)morpholino)methanone | | | | |
| I-226A | ((1S,3R,4S)-3-amino-4- | 26 | 3 | 1.87 | 518.2 |
| | hydroxycyclopentyl)((R)-2-((R)-1-(3-chloro-2- | | | | |
| | (pyridin-3-yl)phenyl)-1-hydroxy-5- | | | | |
| | methoxypentyl)morpholino)methanone | | | | |
| I-226B^{a} | ((1S,3R,4S)-3-amino-4- | 26 | 3 | 1.87 | 518.2 |
| | hydroxycyclopentyl)((R)-2-((S)-1-(3-chloro-2- | | | | |
| | (pyridin-3-yl)phenyl)-1-hydroxy-5- | | | | |
| | methoxypentyl)morpholino)methanone | | | | |
| I-227A | ((1S,3R,4S)-3-amino-4-hydroxycyclopentyl)(2- | 26 | 3 | 2.12 | 523.2 |
| | (1-(3-chloro-2-(3-methyl-1,2,4-oxadiazol-5- | | | | |
| | yl)phenyl)-1-hydroxy-5- | | | | |
| | methoxypentyl)morpholino)methanone | | | | |
| I-228A | methyl 4-((R)-1-((1R,3S)-3- | 5 | 3 | 2.35 | 524.3 |
| | aminocyclopentanecarbonyl)piperidin-3-yl)-4- | | | | |
| | hydroxy-4-(2-(o-tolyloxy)phenyl)butylcarbamate | | | | |
| I-229A | N-(4-((R)-1-((1S,3R,4S)-3-amino-4- | 26 | 3 | 2.07 | 524.3 |
| | hydroxycyclopentanecarbonyl)piperidin-3-yl)-4- | | | | |
| | hydroxy-4-(2-(o-tolyloxy)phenyl)butyl)acetamide | | | | |
| I-230A | ((1S,3R,4S)-3-amino-4- | 26 | | | 525.4 |
| | hydroxycyclopentyl)((R)-3-((S)-1-(2-(2,6- | | | | |
| | dimethylphenoxy)phenyl)-1-hydroxy-5- | | | | |
| | methoxypentyl)piperidin-1-yl)methanone | | | | |
| I-231A | ((1R,3S)-3-aminocyclopentyl)((3R)-3-(1-(6- | 5 | 3 | 2.41 | 527.3 |
| | fluoro-3′-methoxy-5′-methylbiphenyl-2-yl)-1- | | | | |
| | hydroxy-5-methoxypentyl)piperidin-1- | | | | |
| | yl)methanone | | | | |
| I-232A | ((1R,3S)-3-aminocyclopentyl)((3R)-3-(1-(6- | 5 | 3 | 2.56 | 527.3 |
| | chloro-3′-ethylbiphenyl-2-yl)-1-hydroxy-5- | | | | |
| | methoxypentyl)piperidin-1-yl)methanone | | | | |
| I-233A | ((1R,3S)-3-aminocyclopentyl)((3R)-3-(1-(3- | 5 | 3 | 2.56 | 527.3 |
| | chloro-2-(2-methylbenzyl)phenyl)-1-hydroxy-5- | | | | |
| | methoxypentyl)piperidin-1-yl)methanone | | | | |
| I-233B^{a} | ((1R,3S)-3-aminocyclopentyl)((3R)-3-(1-(3- | 5 | 3 | 2.51 | 527.3 |
| | chloro-2-(2-methylbenzyl)phenyl)-1-hydroxy-5- | | | | |
| | methoxypentyl)piperidin-1-yl)methanone | | | | |
| I-234A | ((1R,3S)-3-aminocyclopentyl)((3R)-3-(1-(3- | 5 | 3 | 2.47 | 527.3 |
| | chloro-2-(3-methylbenzyl)phenyl)-1-hydroxy-5- | | | | |
| | methoxypentyl)piperidin-1-yl)methanone | | | | |
| I-234B^{a} | ((1R,3S)-3-aminocyclopentyl)((3R)-3-(1-(3- | 5 | 3 | 2.44 | 527.3 |
| | chloro-2-(3-methylbenzyl)phenyl)-1-hydroxy-5- | | | | |
| | methoxypentyl)piperidin-1-yl)methanone | | | | |
| I-235A | ((1R,3S)-3-aminocyclopentyl)(2-(1-(6-fluoro-3′- | 5 | 3 | 2.31 | 529.3 |
| | methoxy-5′-methylbiphenyl-2-yl)-1-hydroxy-5- | | | | |
| | methoxypentyl)morpholino)methanone | | | | |
| I-236A | ((1R,3S)-3-aminocyclopentyl)((2R)-2-(1-(6- | 5 | 3 | 2.45 | 529.2 |
| | chloro-3′-ethylbiphenyl-2-yl)-1-hydroxy-5- | | | | |
| | methoxypentyl)morpholino)methanone | | | | |
| I-237A | ((1R,3S)-3-aminocyclopentyl)((3R)-3-(1-(3- | 5 | 5 | 2.94; | 529.2 |
| | chloro-2-(o-tolyloxy)phenyl)-1-hydroxy-5- | | | 3.03 | |
| | methoxypentyl)piperidin-1-yl)methanone | | | | |
| I-238A | ((1R,3S)-3-aminocyclopentyl)((3R)-3-(1-(2-(2- | 5 | 5 | 1.97 | 529.3 |
| | chloro-6-methylphenoxy)phenyl)-1-hydroxy-5- | | | | |
| | methoxypentyl)piperidin-1-yl)methanone | | | | |
| I-239A | N-(4-((R)-1-(4-(aminomethyl)benzoyl)piperidin- | 22 | 3 | 2.08 | 530.2 |
| | 3-yl)-4-hydroxy-4-(2-(o- | | | | |
| | tolyloxy)phenyl)butyl)acetamide | | | | |
| I-239B^{a} | N-(4-((R)-1-(4-(aminomethyl)benzoyl)piperidin- | 22 | 3 | 2.08 | 530.2 |
| | 3-yl)-4-hydroxy-4-(2-(o- | | | | |
| | tolyloxy)phenyl)butyl)acetamide | | | | |
| I-240A | ((1S,3R,4S)-3-amino-4- | 26 | 3 | 2.26 | 533.3 |
| | hydroxycyclopentyl)((R)-2-((R)-1-(4′,6-difluoro- | | | | |
| | 3′-methylbiphenyl-2-yl)-1-hydroxy-5- | | | | |
| | methoxypentyl)morpholino)methanone | | | | |
| I-240B^{a} | ((1S,3R,4S)-3-amino-4- | 26 | 3 | 2.31 | 533.2 |
| | hydroxycyclopentyl)((R)-2-((S)-1-(4′,6-difluoro- | | | | |
| | 3′-methylbiphenyl-2-yl)-1-hydroxy-5- | | | | |
| | methoxypentyl)morpholino)methanone | | | | |
| I-241A | (4-(aminomethyl)phenyl)((3R)-3-(1-(3′-ethyl-6- | 22 | 3 | 2.45 | 533.3 |
| | fluorobiphenyl-2-yl)-1-hydroxy-5- | | | | |
| | methoxypentyl)piperidin-1-yl)methanone | | | | |
| I-242A | (3-(aminomethyl)phenyl)(4-(1-(3′-ethyl-6- | 22 | 3 | 2.53 | 533.3 |
| | fluorobiphenyl-2-yl)-1-hydroxy-5- | | | | |
| | methoxypentyl)piperidin-1-yl)methanone | | | | |
| I-243A | (4-(aminomethyl)phenyl)((R)-2-((R)-1-(4′,6- | 22 | 3 | 2.34 | 539.2 |
| | difluoro-3′-methylbiphenyl-2-yl)-1-hydroxy-5- | | | | |
| | methoxypentyl)morpholino)methanone | | | | |
| I-244A | methyl 4-((R)-1-((1S,3R,4S)-3-amino-4- | 26 | 3 | 2.31 | 540.2 |
| | hydroxycyclopentanecarbonyl)piperidin-3-yl)-4- | | | | |
| | hydroxy-4-(2-(o-tolyloxy)phenyl)butylcarbamate | | | | |
| I-244B | methyl 4-((R)-1-((1S,3R,4S)-3-amino-4- | 26 | 3 | 2.31 | 540.2 |
| | hydroxycyclopentanecarbonyl)piperidin-3-yl)-4- | | | | |
| | hydroxy-4-(2-(o-tolyloxy)phenyl)butylcarbamate | | | | |
| I-245A | methyl 4-((R)-1-((1R,2S)-2- | 24 | 3 | 2.40 | 540.3 |
| | aminocyclopentanecarbonyl)piperidin-3-yl)-4- | | | | |
| | (3′-ethyl-6-fluorobiphenyl-2-yl)-4- | | | | |
| | hydroxybutylcarbamate | | | | |
| I-246A | methyl 4-((R)-1-((1R,3S)-3- | 5 | 3 | 2.32 | 540.3 |
| | aminocyclopentanecarbonyl)piperidin-3-yl)-4- | | | | |
| | (3′-ethyl-6-fluorobiphenyl-2-yl)-4- | | | | |
| | hydroxybutylcarbamate | | | | |
| I-247A | (trans-4-aminocyclohexyl)((3R)-3-(1-(6-chloro- | 24 | 3 | 2.60 | 555.3 |
| | 3′-ethylbiphenyl-2-yl)-1-hydroxy-5- | | | | |
| | methoxypentyl)piperidin-1-yl)methanone | | | | |
| I-248A | methyl (R)-4-(3′-ethyl-6-fluorobiphenyl-2-yl)-4- | 24 | 3 | 2.28 | 542.3 |
| | hydroxy-4-((R)-1-((2S,4R)-4-hydroxypyrrolidine- | | | | |
| | 2-carbonyl)piperidin-3-yl)butylcarbamate | | | | |
| I-249A | ((1S,3R,4S)-3-amino-4- | 26 | 3 | 2.35 | 543.3 |
| | hydroxycyclopentyl)((3R)-3-(1-(6-fluoro-3′- | | | | |
| | methoxy-5′-methylbiphenyl-2-yl)-1-hydroxy-5- | | | | |
| | methoxypentyl)piperidin-1-yl)methanone | | | | |
| I-250A | ((1S,3R,4S)-3-amino-4- | 26 | 3 | 2.52 | 543.2 |
| | hydroxycyclopentyl)((3R)-3-(1-(6-chloro-3′- | | | | |
| | ethylbiphenyl-2-yl)-1-hydroxy-5- | | | | |
| | methoxypentyl)piperidin-1-yl)methanone | | | | |
| I-251A | ((1R,3S)-3-aminocyclopentyl)((3R)-3-(1-(3- | 5 | 5 | 3.14; | 543.3 |
| | chloro-2-(2-ethylphenoxy)phenyl)-1-hydroxy-5- | | | 3.38 | |
| | methoxypentyl)piperidin-1-yl)methanone | | | | |
| I-252A | ((1R,3S)-3-aminocyclopentyl)((3R)-3-(1-(3- | 5 | 5 | 2.05 | 543.1 |
| | chloro-2-(3-ethylphenoxy)phenyl)-1-hydroxy-5- | | | | |
| | methoxypentyl)piperidin-1-yl)methanone | | | | |
| I-253A | ((1S,3R,4S)-3-amino-4- | 26 | 3 | 2.48 | 543.2 |
| | hydroxycyclopentyl)((3R)-3-(1-(3-chloro-2-(2- | | | | |
| | methylbenzyl)phenyl)-1-hydroxy-5- | | | | |
| | methoxypentyl)piperidin-1-yl)methanone | | | | |
| I-253B^{a} | ((1S,3R,4S)-3-amino-4- | 26 | 3 | 2.44 | 543.2 |
| | hydroxycyclopentyl)((3R)-3-(1-(3-chloro-2-(2- | | | | |
| | methylbenzyl)phenyl)-1-hydroxy-5- | | | | |
| | methoxypentyl)piperidin-1-yl)methanone | | | | |
| I-254A | ((1S,3R,4S)-3-amino-4- | 26 | 3 | 2.44 | 543.2 |
| | hydroxycyclopentyl)((3R)-3-(1-(3-chloro-2-(3- | | | | |
| | methylbenzyl)phenyl)-1-hydroxy-5- | | | | |
| | methoxypentyl)piperidin-1-yl)methanone | | | | |
| I-254B^{a} | ((1S,3R,4S)-3-amino-4- | 26 | 3 | 2.44 | 543.2 |
| | hydroxycyclopentyl)((3R)-3-(1-(3-chloro-2-(3- | | | | |
| | methylbenzyl)phenyl)-1-hydroxy-5- | | | | |
| | methoxypentyl)piperidin-1-yl)methanone | | | | |
| I-255A | ((1S,3R,4S)-3-amino-4-hydroxycyclopentyl)(2- | 26 | 3 | 2.27 | 545.2 |
| | (1-(6-fluoro-3′-methoxy-5′-methylbiphenyl-2-yl)- | | | | |
| | 1-hydroxy-5- | | | | |
| | methoxypentyl)morpholino)methanone | | | | |
| I-256A | ((1S,3R,4S)-3-amino-4- | 26 | 5 | 2.89; | 545.3 |
| | hydroxycyclopentyl)((3R)-3-(1-(3-chloro-2-(o- | | | 2.92 | |
| | tolyloxy)phenyl)-1-hydroxy-5- | | | | |
| | methoxypentyl)piperidin-1-yl)methanone | | | | |
| I-258A | methyl 4-((R)-1-(4- | 22 | 3 | 2.21 | 546.2 |
| | (aminomethyl)benzoyl)piperidin-3-yl)-4- | | | | |
| | hydroxy-4-(2-(o-tolyloxy)phenyl)butylcarbamate | | | | |
| I-258B^{a} | methyl 4-((R)-1-(4- | 22 | 3 | 2.21 | 546.2 |
| | (aminomethyl)benzoyl)piperidin-3-yl)-4- | | | | |
| | hydroxy-4-(2-(o-tolyloxy)phenyl)butylcarbamate | | | | |
| I-259A | (4-(2-aminoethyl)phenyl)(4-(1-(3′-ethyl-6- | 22 | 3 | 2.54 | 547.3 |
| | fluorobiphenyl-2-yl)-1-hydroxy-5- | | | | |
| | methoxypentyl)piperidin-1-yl)methanone | | | | |
| I-260A | (4-(aminomethyl)phenyl)((3R)-3-(1-(6-fluoro-3′- | 22 | 3 | 2.36 | 549.3 |
| | methoxy-5′-methylbiphenyl-2-yl)-1-hydroxy-5- | | | | |
| | methoxypentyl)piperidin-1-yl)methanone | | | | |
| I-261A | ((1S,3R,4S)-3-amino-4- | 26 | 3 | 2.32 | 549.3 |
| | hydroxycyclopentyl)((2R)-2-((1R)-1-(6-chloro-2′- | | | | |
| | fluoro-5′-methylbiphenyl-2-yl)-1-hydroxy-5- | | | | |
| | methoxypentyl)morpholino)methanone | | | | |
| I-262A | (4-(aminomethyl)phenyl)((3R)-3-(1-(6-chloro-3′- | 24 | 3 | 2.54 | 549.2 |
| | ethylbiphenyl-2-yl)-1-hydroxy-5- | | | | |
| | methoxypentyl)piperidin-1-yl)methanone | | | | |
| I-263A | (4-(aminomethyl)phenyl)((3R)-3-(1-(3-chloro-2- | 22 | 3 | 2.48 | 549.2 |
| | (2-methylbenzyl)phenyl)-1-hydroxy-5- | | | | |
| | methoxypentyl)piperidin-1-yl)methanone | | | | |
| I-264A | (4-(aminomethyl)phenyl)((3R)-3-(1-(3-chloro-2- | 22 | 3 | 2.43 | 549.2 |
| | (3-methylbenzyl)phenyl)-1-hydroxy-5- | | | | |
| | methoxypentyl)piperidin-1-yl)methanone | | | | |
| I-265A | ((1R,3S)-3-aminocyclopentyl)((R)-3-((S)-1-(3- | 5 | 3 | 2.20 | 550.3 |
| | chloro-2-(quinolin-3-yl)phenyl)-1-hydroxy-5- | | | | |
| | methoxypentyl)piperidin-1-yl)methanone | | | | |
| I-265B^{a} | ((1R,3S)-3-aminocyclopentyl)((R)-3-((R)-1-(3- | 5 | 3 | 2.07 | 550.2 |
| | chloro-2-(quinolin-3-yl)phenyl)-1-hydroxy-5- | | | | |
| | methoxypentyl)piperidin-1-yl)methanone | | | | |
| I-266A | (6-(aminomethyl)pyridin-3-yl)((3R)-3-(1-(6- | 24 | 3 | 2.51 | 550.2 |
| | chloro-3′-ethylbiphenyl-2-yl)-1-hydroxy-5- | | | | |
| | methoxypentyl)piperidin-1-yl)methanone | | | | |
| I-267A | (4-(aminomethyl)phenyl)(2-(1-(6-fluoro-3′- | 22 | 3 | 2.29 | 551.2 |
| | methoxy-5′-methylbiphenyl-2-yl)-1-hydroxy-5- | | | | |
| | methoxypentyl)morpholino)methanone | | | | |
| I-268A | (3-(aminomethyl)phenyl)((R)-2-((R)-1-(6-chloro- | 24 | 3 | 2.44 | 551.2 |
| | 3′-ethylbiphenyl-2-yl)-1-hydroxy-5- | | | | |
| | methoxypentyl)morpholino)methanone | | | | |
| I-269A | (4-(aminomethyl)phenyl)((R)-2-((R)-1-(6-chloro- | 22 | 3 | 2.41 | 551.2 |
| | 3′-ethylbiphenyl-2-yl)-1-hydroxy-5- | | | | |
| | methoxypentyl)morpholino)methanone | | | | |
| I-270A | ((1R,3S)-3-aminocyclopentyl)((R)-2-((R)-1-(3- | 5 | 3 | 2.44 | 551.2 |
| | chloro-2-(naphthalen-2-yl)phenyl)-1-hydroxy-5- | | | | |
| | methoxypentyl)morpholino)methanone | | | | |
| I-271A | (4-(aminomethyl)phenyl)((3R)-3-(1-(3-chloro-2- | 22 | 5 | 2.90; | 551.7 |
| | (o-tolyloxy)phenyl)-1-hydroxy-5- | | | 3.0 | |
| | methoxypentyl)piperidin-1-yl)methanone | | | | |
| I-272A | (4-(aminomethyl)phenyl)((3R)-3-(1-(2-(2-chloro- | 22 | | | 551.1 |
| | 6-methylphenoxy)phenyl)-1-hydroxy-5- | | | | |
| | methoxypentyl)piperidin-1-yl)methanone | | | | |
| I-273A | ((1R,3S)-3-aminocyclopentyl)(2-(1-(3-chloro-2- | 5 | 3 | 2.10 | 552.2 |
| | (quinolin-3-yl)phenyl)-1-hydroxy-5- | | | | |
| | methoxypentyl)morpholino)methanone | | | | |
| I-274A | (6-(aminomethyl)pyridin-3-yl)((R)-2-((R)-1-(6- | 22 | 3 | 2.42 | 552.2 |
| | chloro-3′-ethylbiphenyl-2-yl)-1-hydroxy-5- | | | | |
| | methoxypentyl)morpholino)methanone | | | | |
| I-275A | methyl ((4S)-4-hydroxy-4-((3R)-1- | | 3 | 2.36 | 538.2 |
| | {[(1R,3S)-3- | | | | |
| | (methylamino)cyclopentyl]carbonyl}-3- | | | | |
| | piperidinyl)-4-{2-[(2- | | | | |
| | methylphenyl)oxy]phenyl}butyl)carbamate | | | | |
| I-276A | (4-(aminomethyl)phenyl)((2R)-2-((1R)-1-(6- | 22 | 3 | 2.37 | 555.3 |
| | chloro-2′-fluoro-5′-methylbiphenyl-2-yl)-1- | | | | |
| | hydroxy-5- | | | | |
| | methoxypentyl)morpholino)methanone | | | | |
| I-277A | methyl 4-(6-fluoro-3′-methoxybiphenyl-2-yl)-4- | 22 | 3 | 2.41 | 556.3 |
| | hydroxy-4-((R)-1-((1R,3S)-3- | | | | |
| | (methylamino)cyclopentanecarbonyl)piperidin- | | | | |
| | 3-yl)butylcarbamate | | | | |
| I-278A | N-((S)-4-((R)-1-((1S,3R,4S)-3-amino-4- | 26 | 3 | 2.27 | 556.3 |
| | hydroxycyclopentanecarbonyl)piperidin-3-yl)-4- | | | | |
| | (6-chloro-3′-ethylbiphenyl-2-yl)-4- | | | | |
| | hydroxybutyl)acetamide | | | | |
| I-279A | methyl (S)-4-((R)-1-((1R,3S)-3- | 5 | 3 | 2.42 | 556.2 |
| | aminocyclopentanecarbonyl)piperidin-3-yl)-4- | | | | |
| | (6-chloro-3′-ethylbiphenyl-2-yl)-4- | | | | |
| | hydroxybutylcarbamate | | | | |
| I-280A | methyl 4-((R)-4-((1R,3S)-3- | 5 | 3 | 2.31 | 558.2 |
| | aminocyclopentanecarbonyl)morpholin-2-yl)-4- | | | | |
| | (6-chloro-3′-ethylbiphenyl-2-yl)-4- | | | | |
| | hydroxybutylcarbamate | | | | |
| I-281A | ((1S,3R,4S)-3-amino-4- | 26 | 5 | 1.82 | 559.0 |
| | hydroxycyclopentyl)((3R)-3-(1-(3-chloro-2-(3- | | | | |
| | ethylphenoxy)phenyl)-1-hydroxy-5- | | | | |
| | methoxypentyl)piperidin-1-yl)methanone | | | | |
| I-282A | methyl 4-hydroxy-4-((R)-1-(4- | 22 | 3 | 2.49 | 560.3 |
| | ((methylamino)methyl)benzoyl)piperidin-3-yl)-4- | | | | |
| | (2-(o-tolyloxy)phenyl)butylcarbamate | | | | |
| I-283A | ((1S,3R,4S)-3-amino-4- | 26 | 3 | 2.17 | 561.2 |
| | hydroxycyclopentyl)((R)-2-((R)-1-(6-fluoro-3′,5′- | | | | |
| | dimethoxybiphenyl-2-yl)-1-hydroxy-5- | | | | |
| | methoxypentyl)morpholino)methanone | | | | |
| I-284A | ((1S,3R,4S)-3-amino-4- | 26 | 4 | 1.64 | 561.3 |
| | hydroxycyclopentyl)((R)-2-((R)-1-(6-chloro-3′- | | | | |
| | (methoxymethyl)biphenyl-2-yl)-1-hydroxy-5- | | | | |
| | methoxypentyl)morpholino)methanone | | | | |
| I-285A | (4-(aminomethyl)cyclohexyl)((2R)-2-((1R)-1-(6- | 24 | 3 | 3.03 | 561.3 |
| | chloro-2′-fluoro-5′-methylbiphenyl-2-yl)-1- | | | | |
| | hydroxy-5- | | | | |
| | methoxypentyl)morpholino)methanone | | | | |
| I-286A | N-((S)-4-(6-chloro-3′-ethylbiphenyl-2-yl)-4- | 22 | 3 | 2.42 | 562.2 |
| | hydroxy-4-((R)-1-(4- | | | | |
| | ((methylamino)methyl)benzoyl)piperidin-3- | | | | |
| | yl)butyl)formamide | | | | |
| I-287A | (4-(2-aminoethyl)phenyl)((3R)-3-(1-(6-chloro-3′- | 24 | 3 | 2.55 | 563.2 |
| | ethylbiphenyl-2-yl)-1-hydroxy-5- | | | | |
| | methoxypentyl)piperidin-1-yl)methanone | | | | |
| I-288A | ((3R)-3-(1-(3-chloro-2-(2-methylbenzyl)phenyl)- | 22 | 3 | 2.49 | 563.3 |
| | 1-hydroxy-5-methoxypentyl)piperidin-1-yl)(4- | | | | |
| | ((methylamino)methyl)phenyl)methanone | | | | |
| I-288B^{a} | ((3R)-3-(1-(3-chloro-2-(2-methylbenzyl)phenyl)- | 22 | 3 | 2.44 | 563.3 |
| | 1-hydroxy-5-methoxypentyl)piperidin-1-yl)(4- | | | | |
| | ((methylamino)methyl)phenyl)methanone | | | | |
| I-289A | methyl 4-((R)-1-(4- | 22 | 3 | 2.18 | 564.1 |
| | (aminomethyl)benzoyl)piperidin-3-yl)-4-(6- | | | | |
| | fluoro-3′-methoxybiphenyl-2-yl)-4- | | | | |
| | hydroxybutylcarbamate | | | | |
| I-290A | (4-(aminomethyl)phenyl)((3R)-3-(1-(3-chloro-2- | 22 | 5) | 3.06; | 565.3 |
| | (2-ethylphenoxy)phenyl)-1-hydroxy-5- | | | 3.15 | |
| | methoxypentyl)piperidin-1-yl)methanone | | | | |
| I-291A | (4-(aminomethyl)phenyl)((3R)-3-(1-(3-chloro-2- | 22 | 5 | 1.83 | 565.3 |
| | (3-ethylphenoxy)phenyl)-1-hydroxy-5- | | | | |
| | methoxypentyl)piperidin-1-yl)methanone | | | | |
| I-292A | ((1S,3R,4S)-3-amino-4- | 26 | 3 | 2.12 | 566.2 |
| | hydroxycyclopentyl)((R)-3-((R)-1-(3-chloro-2- | | | | |
| | (quinolin-3-yl)phenyl)-1-hydroxy-5- | | | | |
| | methoxypentyl)piperidin-1-yl)methanone | | | | |
| I-292B^{a} | ((1S,3R,4S)-3-amino-4- | 26 | 3 | 2.36 | 566.2 |
| | hydroxycyclopentyl)((R)-3-((S)-1-(3-chloro-2- | | | | |
| | (quinolin-3-yl)phenyl)-1-hydroxy-5- | | | | |
| | methoxypentyl)piperidin-1-yl)methanone | | | | |
| I-293A | (4-(aminomethyl)phenyl)((R)-2-((R)-1-(6-chloro- | 22 | 4 | 1.61 | 567.5 |
| | 3′-(methoxymethyl)biphenyl-2-yl)-1-hydroxy-5- | | | | |
| | methoxypentyl)morpholino)methanone | | | | |
| I-294A | ((1S,3R,4S)-3-amino-4- | 26 | 3 | 2.41 | 567.2 |
| | hydroxycyclopentyl)((R)-2-((R)-1-(3-chloro-2- | | | | |
| | (naphthalen-2-yl)phenyl)-1-hydroxy-5- | | | | |
| | methoxypentyl)morpholino)methanone | | | | |
| I-295A | ((1S,3R,4S)-3-amino-4-hydroxycyclopentyl)(2- | 26 | 3 | 2.07 | 568.2 |
| | (1-(3-chloro-2-(quinolin-3-yl)phenyl)-1-hydroxy- | | | | |
| | 5-methoxypentyl)morpholino)methanone | | | | |
| I-295B^{a} | ((1S,3R,4S)-3-amino-4- | 26 | 3 | 2.09 | 552.2 |
| | hydroxycyclopentyl)((R)-2-((S)-1-(3-chloro-2- | | | | |
| | (quinolin-3-yl)phenyl)-1-hydroxy-5- | | | | |
| | methoxypentyl)morpholino)methanone | | | | |
| I-295C^{a} | ((1S,3R,4S)-3-amino-4- | 26 | 3 | 2.06 | 568.2 |
| | hydroxycyclopentyl)((R)-2-((R)-1-(3-chloro-2- | | | | |
| | (quinolin-3-yl)phenyl)-1-hydroxy-5- | | | | |
| | methoxypentyl)morpholino)methanone | | | | |
| I-296A | ((1S,3R,4S)-3-amino-4- | 26 | 3 | 2.09 | 568.2 |
| | hydroxycyclopentyl)((2R)-2-((1R)-1-(3-chloro-2- | | | | |
| | (isoquinolin-4-yl)phenyl)-1-hydroxy-5- | | | | |
| | methoxypentyl)morpholino)methanone | | | | |
| I-296B^{a} | ((1S,3R,4S)-3-amino-4- | 26 | 3 | 2.09 | 568.2 |
| | hydroxycyclopentyl)((2S)-2-((1R)-1-(3-chloro-2- | | | | |
| | (isoquinolin-4-yl)phenyl)-1-hydroxy-5- | | | | |
| | methoxypentyl)morpholino)methanone | | | | |
| I-297A | methyl 4-(6-chloro-3′-ethylbiphenyl-2-yl)-4- | 24 | 4 | 1.80 | 570.5 |
| | hydroxy-4-((R)-1-((1R,3S)-3- | | | | |
| | (methylamino)cyclopentanecarbonyl)piperidin- | | | | |
| | 3-yl)butylcarbamate | | | | |
| I-298A | methyl 4-((R)-1-((1R,3S)-3- | 5 | 3 | 2.40 | 570.2 |
| | aminocyclopentanecarbonyl)piperidin-3-yl)-4- | | | | |
| | (6-chloro-3′-isopropylbiphenyl-2-yl)-4- | | | | |
| | hydroxybutylcarbamate | | | | |
| I-299A | methyl (S)-4-((R)-1-((1S,3R,4S)-3-amino-4- | 26 | 3 | 2.35 | 572.3 |
| | hydroxycyclopentanecarbonyl)piperidin-3-yl)-4- | | | | |
| | (6-chloro-3′-ethylbiphenyl-2-yl)-4- | | | | |
| | hydroxybutylcarbamate | | | | |
| I-300A | methyl 4-(6-chloro-3′-methoxybiphenyl-2-yl)-4- | 22 | 3 | 2.40 | 572.2 |
| | hydroxy-4-((R)-1-((1R,3S)-3- | | | | |
| | (methylamino)cyclopentanecarbonyl)piperidin- | | | | |
| | 3-yl)butylcarbamate | | | | |
| I-301A | (4-(aminomethyl)phenyl)((R)-3-((R)-1-(3-chloro- | 22 | 3 | 2.02; | 572.2 |
| | 2-(quinolin-3-yl)phenyl)-1-hydroxy-5- | | | 2.16 | |
| | methoxypentyl)piperidin-1-yl)methanone | | | | |
| I-302A | (4-(aminomethyl)phenyl)((R)-2-((R)-1-(3-chloro- | 22 | 3 | 2.42 | 573.2 |
| | 2-(naphthalen-2-yl)phenyl)-1-hydroxy-5- | | | | |
| | methoxypentyl)morpholino)methanone | | | | |
| I-303A | (4-(aminomethyl)phenyl)((R)-2-((S)-1-(3-chloro- | 22 | 3 | 2.07 | 524.3 |
| | 2-(quinolin-3-yl)phenyl)-1-hydroxy-5- | | | | |
| | methoxypentyl)morpholino)methanone | | | | |
| I-303B^{a} | (4-(aminomethyl)phenyl)((R)-2-((S)-1-(3-chloro- | 22 | 3 | 2.11 | 574.2 |
| | 2-(quinolin-3-yl)phenyl)-1-hydroxy-5- | | | | |
| | methoxypentyl)morpholino)methanone | | | | |
| I-304A | N-((S)-4-(6-chloro-3′-ethylbiphenyl-2-yl)-4- | 22 | 3 | 2.45 | 576.2 |
| | hydroxy-4-((R)-1-(4- | | | | |
| | ((methylamino)methyl)benzoyl)piperidin-3- | | | | |
| | yl)butyl)acetamide | | | | |
| I-305A | ((1S,3R,4S)-3-amino-4- | 26 | 3 | 2.31 | 577.3 |
| | hydroxycyclopentyl)((R)-2-((R)-1-(6-chloro-3′,5′- | | | | |
| | dimethoxybiphenyl-2-yl)-1-hydroxy-5- | | | | |
| | methoxypentyl)morpholino)methanone | | | | |
| I-306A | methyl 4-(6-fluoro-3′-methoxybiphenyl-2-yl)-4- | 22 | 3 | 2.34 | 578.2 |
| | hydroxy-4-((R)-1-(4- | | | | |
| | ((methylamino)methyl)benzoyl)piperidin-3- | | | | |
| | yl)butylcarbamate | | | | |
| I-307A | methyl 4-((R)-1-(4- | 23 | 3 | 2.44 | 578.3 |
| | (aminomethyl)benzoyl)piperidin-3-yl)-4-(6- | | | | |
| | chloro-3′-ethylbiphenyl-2-yl)-4- | | | | |
| | hydroxybutylcarbamate | | | | |
| I-307B^{a} | methyl (R)-4-((R)-1-(4- | 22 | 4 | 1.74 | 578.4 |
| | (aminomethyl)benzoyl)piperidin-3-yl)-4-(6- | | | | |
| | chloro-3′-ethylbiphenyl-2-yl)-4- | | | | |
| | hydroxybutylcarbamate | | | | |
| I-308A | methyl 4-(6-chloro-3′-methylbiphenyl-2-yl)-4- | 22 | 3 | 2.50 | 578.2 |
| | hydroxy-4-((R)-1-(4- | | | | |
| | ((methylamino)methyl)benzoyl)piperidin-3- | | | | |
| | yl)butylcarbamate | | | | |
| I-309A | methyl 4-((R)-1-(6- | 22 | 3 | 2.40 | 579.2 |
| | (aminomethyl)nicotinoyl)piperidin-3-yl)-4-(6- | | | | |
| | chloro-3′-ethylbiphenyl-2-yl)-4- | | | | |
| | hydroxybutylcarbamate | | | | |
| I-310A | methyl (R)-4-((R)-4-(4- | 22 | 3 | 2.31 | 580.2 |
| | (aminomethyl)benzoyl)morpholin-2-yl)-4-(6- | | | | |
| | chloro-3′-ethylbiphenyl-2-yl)-4- | | | | |
| | hydroxybutylcarbamate | | | | |
| I-311A | methyl 4-((R)-1-(4- | 22 | 3 | 2.37 | 580.2 |
| | (aminomethyl)benzoyl)piperidin-3-yl)-4-(6- | | | | |
| | chloro-3′-methoxybiphenyl-2-yl)-4- | | | | |
| | hydroxybutylcarbamate | | | | |
| I-312A | methyl 4-((R)-1-(trans-4- | 24 | 3 | 2.43 | 584.3 |
| | (aminomethyl)cyclohexanecarbonyl)piperidin-3- | | | | |
| | yl)-4-(6-chloro-3′-ethylbiphenyl-2-yl)-4- | | | | |
| | hydroxybutylcarbamate | | | | |
| I-313A | methyl 4-((R)-1-((1S,3R,4S)-3-amino-4- | 26 | 3 | 2.37 | 586.2 |
| | hydroxycyclopentanecarbonyl)piperidin-3-yl)-4- | | | | |
| | (6-chloro-3′-isopropylbiphenyl-2-yl)-4- | | | | |
| | hydroxybutylcarbamate | | | | |
| I-314A | N-((S)-4-(6-chloro-3′-ethylbiphenyl-2-yl)-4- | 21 | 3 | 2.48 | 590.3 |
| | hydroxy-4-((R)-1-(4- | | | | |
| | ((methylamino)methyl)benzoyl)piperidin-3- | | | | |
| | yl)butyl)propionamide | | | | |
| I-315A | ethyl 4-((R)-1-(4- | 22 | 4 | 1.73 | 592.3 |
| | (aminomethyl)benzoyl)piperidin-3-yl)-4-(6- | | | | |
| | chloro-3′-ethylbiphenyl-2-yl)-4- | | | | |
| | hydroxybutylcarbamate | | | | |
| I-316A | methyl 4-(6-chloro-3′-ethylbiphenyl-2-yl)-4- | 24 | 4 | 1.77 | 592.3 |
| | hydroxy-4-((R)-1-(4- | | | | |
| | ((methylamino)methyl)benzoyl)piperidin-3- | | | | |
| | yl)butylcarbamate | | | | |
| I-317A | methyl 4-((R)-1-(4- | 22 | 3 | 2.45 | 592.3 |
| | (aminomethyl)benzoyl)piperidin-3-yl)-4-(6- | | | | |
| | chloro-3′-isopropylbiphenyl-2-yl)-4- | | | | |
| | hydroxybutylcarbamate | | | | |
| I-318A | N-((S)-4-(6-chloro-3′-ethylbiphenyl-2-yl)-4- | 22 | 3 | 2.40 | 592.2 |
| | hydroxy-4-((R)-1-(4- | | | | |
| | ((methylamino)methyl)benzoyl)piperidin-3- | | | | |
| | yl)butyl)-2-hydroxyacetamide | | | | |
| I-319A | methyl 4-(6-chloro-3′-methoxybiphenyl-2-yl)-4- | 22 | 3 | 2.39 | 594.2 |
| | hydroxy-4-((R)-1-(4- | | | | |
| | ((methylamino)methyl)benzoyl)piperidin-3- | | | | |
| | yl)butylcarbamate | | | | |
| I-320A | ((1R,3S)-3-aminocyclopentyl)((R)-3-((S)-1-(3′- | 5 | 3 | 2.61 | 595.3 |
| | ethoxy-6-fluoro-5′-(trifluoromethyl)biphenyl-2- | | | | |
| | yl)-1-hydroxy-5-methoxypentyl)piperidin-1- | | | | |
| | yl)methanone | | | | |
| I-321A | methyl 4-((R)-1-((1S,3R,4S)-3-amino-4- | 26 | 3 | 2.34 | 595.2 |
| | hydroxycyclopentanecarbonyl)piperidin-3-yl)-4- | | | | |
| | (3-chloro-2-(quinolin-3-yl)phenyl)-4- | | | | |
| | hydroxybutylcarbamate | | | | |
| I-322A | methyl 4-((R)-1-(4-(aminomethyl)-2- | 24 | 3 | 2.47 | 596.2 |
| | fluorobenzoyl)piperidin-3-yl)-4-(6-chloro-3′- | | | | |
| | ethylbiphenyl-2-yl)-4-hydroxybutylcarbamate | | | | |
| I-323A | ((1R,3S)-3-aminocyclopentyl)((R)-2-((R)-1-(3′- | 5 | 3 | 2.54 | 597.2 |
| | ethoxy-6-fluoro-5′-(trifluoromethyl)biphenyl-2- | | | | |
| | yl)-1-hydroxy-5- | | | | |
| | methoxypentyl)morpholino)methanone | | | | |
| I-324A | isopropyl 4-((R)-1-(4- | 22 | 4 | 1.87 | 606.4 |
| | (aminomethyl)benzoyl)piperidin-3-yl)-4-(6- | | | | |
| | chloro-3′-ethylbiphenyl-2-yl)-4- | | | | |
| | hydroxybutylcarbamate | | | | |
| I-325A | methyl 4-(6-chloro-3′-ethylbiphenyl-2-yl)-4-((R)- | 24 | 4 | 1.78 | 606.3 |
| | 1-(4-((ethylamino)methyl)benzoyl)piperidin-3- | | | | |
| | yl)-4-hydroxybutylcarbamate | | | | |
| I-326A | ((1S,3R,4S)-3-amino-4- | 26 | 3 | 2.54 | 611.3 |
| | hydroxycyclopentyl)((R)-3-((S)-1-(3′-ethoxy-6- | | | | |
| | fluoro-5′-(trifluoromethyl)biphenyl-2-yl)-1- | | | | |
| | hydroxy-5-methoxypentyl)piperidin-1- | | | | |
| | yl)methanone | | | | |
| I-326B^{a} | ((1S,3R,4S)-3-amino-4- | 26 | 3 | 2.61 | 611.2 |
| | hydroxycyclopentyl)((R)-3-((R)-1-(3′-ethoxy-6- | | | | |
| | fluoro-5′-(trifluoromethyl)biphenyl-2-yl)-1- | | | | |
| | hydroxy-5-methoxypentyl)piperidin-1- | | | | |
| | yl)methanone | | | | |
| I-327A | ((1S,3R,4S)-3-amino-4-hydroxycyclopentyl)(2- | 26 | 3 | 2.50 | 613.2 |
| | (1-(3′-ethoxy-6-fluoro-5′- | | | | |
| | (trifluoromethyl)biphenyl-2-yl)-1-hydroxy-5- | | | | |
| | methoxypentyl)morpholino)methanone | | | | |
| I-328A | methyl 4-(6-chloro-3′-ethylbiphenyl-2-yl)-4- | 24 | 4 | 1.86 | 620.4 |
| | hydroxy-4-((R)-1-(4- | | | | |
| | ((isopropylamino)methyl)benzoyl)piperidin-3- | | | | |
| | yl)butylcarbamate | | | | |
| I-329A | N-((S)-4-(6-chloro-3′-ethylbiphenyl-2-yl)-4- | 22 | 3 | 2.49 | 630.2 |
| | hydroxy-4-((R)-1-(4- | | | | |
| | ((methylamino)methyl)benzoyl)piperidin-3- | | | | |
| | yl)butyl)-2,2,2-trifluoroacetamide | | | | |
| I-330A | ((1R,3S)-3-aminocyclopentyl)((R)-2-((R)-1-(6- | 5 | 3 | 2.21 | 545.2 |
| | fluoro-3′,5′-dimethoxybiphenyl-2-yl)-1-hydroxy- | | | | |
| | 5-methoxypentyl)morpholino)methanone | | | | |
| I-331A | ((1R,3S)-3-aminocyclopentyl)((2R)-2-((1R)-1- | 5 | 3 | 2.07 | 552.2 |
| | (3-chloro-2-(isoquinolin-4-yl)phenyl)-1-hydroxy- | | | | |
| | 5-methoxypentyl)morpholino)methanone | | | | |
| I-332 | methyl (4-((3R)-1-{[(1R,3S)-3- | 5 | 5 | 2.65; | 558.2 |
| | aminocyclopentyl]carbonyl}-3-piperidinyl)-4-{3- | | | 2.78 | |
| | chloro-2-[(2-methylphenyl)oxy]phenyl}-4- | | | | |
| | hydroxybutyl)carbamate | | | | |
| I-333 | methyl (4-((3R)-1-{[(1R,3S)-3- | 5 | 5 | 2.78; | 572.3 |
| | aminocyclopentyl]carbonyl}-3-piperidinyl)-4-{3- | | | 2.92 | |
| | chloro-2-[(2-ethylphenyl)oxy]phenyl}-4- | | | | |
| | hydroxybutyl)carbamate | | | | |
| I-334 | methyl (4-((3R)-1-{[(1S,3R,4S)-3-amino-4- | 27 | 5 | 2.61; | 574.2 |
| | hydroxycyclopentyl]carbonyl}-3-piperidinyl)-4- | | | 2.70 | |
| | {3-chloro-2-[(2-methylphenyl)oxy]phenyl}-4- | | | | |
| | hydroxybutyl)carbamate | | | | |
| I-335 | methyl (4-((3R)-1-{[(1S,3R,4S)-3-amino-4- | 27 | 5 | 2.75; | 588.2 |
| | hydroxycyclopentyl]carbonyl}-3-piperidinyl)-4- | | | 2.86 | |
| | {3-chloro-2-[(2-ethylphenyl)oxy]phenyl}-4- | | | | |
| | hydroxybutyl)carbamate | | | | |
| I-336 | methyl [4-((3R)-1-{[(1S,3R,4S)-3-amino-4- | 27 | 3 | 2.36 | 556.3 |
| | hydroxycyclopentyl]carbonyl}-3-piperidinyl)-4- | | | | |
| | (3′-ethyl-6-fluoro-2-biphenylyl)-4- | | | | |
| | hydroxybutyl]carbamate | | | | |
| I-337 | methyl {4-(6-chloro-3′-ethyl-2-biphenylyl)-4- | 24 | 3 | 2.46 | 598.3 |
| | hydroxy-4-[(3R)-1-({trans-4- | | | | |
| | [(methylamino)methyl]cyclohexyl}carbonyl)-3- | | | | |
| | piperidinyl]butyl}carbamate | | | | |
| I-338 | methyl {4-(3′-ethyl-6-fluoro-2-biphenylyl)-4- | 22 | 3 | 2.45 | 576.3 |
| | hydroxy-4-[(3R)-1-({4- | | | | |
| | [(methylamino)methyl]phenyl}carbonyl)-3- | | | | |
| | piperidinyl]butyl}carbamate | | | | |
| I-339 | methyl {4-(2′,6-difluoro-5′-methyl-2-biphenylyl)- | 22 | 3 | 2.43 | 580.2 |
| | 4-hydroxy-4-[(3R)-1-({4- | | | | |
| | [(methylamino)methyl]phenyl}carbonyl)-3- | | | | |
| | piperidinyl]butyl}carbamate | | | | |
| I-340 | N-{4-(2′,6-difluoro-5′-methyl-2-biphenylyl)-4- | 22 | 3 | 2.48 | 580.2 |
| | hydroxy-4-[(3R)-1-({4- | | | | |
| | [(methylamino)methyl]phenyl}carbonyl)-3- | | | | |
| | piperidinyl]butyl}-2-hydroxyacetamide | | | | |

^{a}Minor isomer separated by chromatography

### Example 28

### In Vitro Activity Studies

### IC₅₀ for Renin

The compounds of the invention have enzyme-inhibiting properties. In particular, they inhibit the action of the natural enzyme renin. The latter passes from the kidneys into the blood where it effects the cleavage of angiotensinogen, releasing the decapeptide angiotensin I which is then cleaved in the blood, lungs, the kidneys and other organs by angiotensin converting enzyme to form the octapeptide angiotensin II. The octapeptide increases blood pressure both directly by binding to its receptor, causing arterial vasoconstriction, and indirectly by liberating from the adrenal glands the sodium-ion-retaining hormone aldosterone, accompanied by an increase in extracellular fluid volume. That increase can be attributed to the action of angiotensin II. Inhibitors of the enzymatic activity of renin bring about a reduction in the formation of angiotensin I. As a result a smaller amount of angiotensin II is produced. The reduced concentration of that active peptide hormone is the direct cause of the hypotensive effect of renin inhibitors.

The action of renin inhibitors in vitro is demonstrated experimentally by means of a test which measures the increase in fluorescence of an internally quenched peptide substrate. The sequence of this peptide corresponds to the sequence of human angiotensinogen. The following test protocol is used: All reactions are carried out in a flat bottom white opaque microtiter plate. A 4 μL aliquot of 400 μM renin substrate (DABCYL-γ-Abu-Ile-His-Pro-Phe-His-Leu-Val-Ile-His-Thr-EDANS) in 192 μL assay buffer (50 mM BES, 150 mM NaCl, 0.25 mg/mL bovine serum albumin, pH7.0) is added to 4 μL of test compound in DMSO at various concentrations ranging from 10 μM to 1 nM final concentrations. Next, 100 μL of trypsin-activated recombinant human renin (final enzyme concentration of 0.2-2 nM) in assay buffer is added, and the solution is mixed by pipetting. The increase in fluorescence at 495 nm (excitation at 340 nm) is measured for 60-360 min at rt using a Perkin-Elmer Fusion microplate reader. The slope of a linear portion of the plot of fluorescence increase as a function of time is then determined, and the rate is used for calculating percent inhibition in relation to uninhibited control. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor. (Wang G. T. et al. Anal. Biochem. 1993, 210, 351; Nakamura, N. et al. J. Biochem. (Tokyo) 1991, 109, 741; Murakami, K. et al. Anal Biochem. 1981, 110, 232).

### Example 29

### In Vitro Activity Studies

### IC₅₀ for Renin

All reactions are carried out in a low volume, black, 384 well microtiter plate (greiner bio-one). Compounds were diluted in 100% DMSO, and a 100 nL aliquot of each compound concentration was stamped into the plate using a Hummingbird (Genomic Solutions). 5 μL of 600 pM renin (trypsin-activated recombinant human renin) was then added to the plate, followed by 5 μL of 2 μM substrate (Arg-Glu-Lys(5-FAM)-Ile-His-Pro-Phe-His-Leu-Val-Ile-His-Thr-Lys(5,6-TAMRA)-Arg-CONH₂). Both renin and substrate were made up in buffer containing 50 mM HEPES, 125 mM NaCl, 0.1% CHAPS, with the pH adjusted to 7.4. After 2 hours of reaction at room temperature, the plates were read on a Viewlux (PerkinElmer) with an excitation/emission of 485/530 nm, and using a 505 nm cutoff filter. The percent inhibition values are plotted as a function of inhibitor concentration, and the IC₅₀ is determined from a fit of this data to a four parameter equation. The IC₅₀ is defined as the concentration of a particular inhibitor that reduces the formation of product by 50% relative to a control sample containing no inhibitor.

### Example 30

### IC₅₀ Values of the Disclosed Compounds for Renin

The IC₅₀ values of the disclosed compounds for renin were determined according to the protocol described in Example 29 or 30. In these in vitro systems the compounds of the invention exhibit 50% inhibition at concentrations of from approximately 5000 nM to approximately 0.01 nM. Preferred compounds of the invention exhibit 50% inhibition at concentrations of from approximately 50 nM to approximately 0.01 nM. More preferred compounds of the invention exhibit 50% inhibition at concentrations of from approximately 5 nM to approximately 0.01 nM. Highly preferred compounds of the invention exhibit 50% inhibition at concentrations of from approximately 5 nM to approximately 0.01 nM and exhibit 50% inhibition at concentrations of from approximately 10 nM to approximately 0.01 nM in the in vitro assay in the presence of human plasma described below.

### Example 31

### In Vitro Activity of the Disclosed Compounds in Human Plasma

The action of renin inhibitors in vitro in human plasma is demonstrated experimentally by the decrease in plasma renin activity (PRA) levels observed in the presence of the compounds. Incubations mixtures contain in the final volume of 250 μL 95.5 mM N,N-bis(2-hydroxyethyl)-2-aminoethanesulfonic acid, pH 7.0, 8 mM EDTA, 0.1 mM neomycin sulfate, 1 mg/ml sodium azide, 1 mM phenylmethanesulfonyl fluoride, 2% DMSO and 87.3% of pooled mixed-gender human plasma stabilized with EDTA. For plasma batches with low PRA (less than 1 ng/ml/hr) ˜2 pM of recombinant human renin IS added to achieve PRA of 3-4 ng/ml/hr. The cleavage of endogenous angiotensinogen in plasma is carried out at 37° C. for 90 min and the product angiotensin I is measured by competitive radioimmunoassay using DiaSorin PRA kit. Uninhibited incubations containing 2% DMSO and fully inhibited controls with 2 μM of isovaleryl-Phe-Nle-Sta-Ala-Sta-OH are used for deriving percent of inhibition for each concentration of inhibitors and fitting dose-response data into a four parametric model from which IC₅₀ values, defined as concentrations of inhibitors at which 50% inhibition occurs, is determined.

### Example 32

### Efficacy of the Disclosed Inhibitors in a Transgenic Rat Model

The efficacy of the renin inhibitors is also evaluated in vivo in double transgenic rats engineered to express human renin and human angiotensinogen (Bohlender J, Fukamizu A, Lippoldt A, Nomura T, Dietz R, Menard J, Murakami K, Luft F C, Ganten D. High human renin hypertension in transgenic rats. Hypertension 1997, 29, 428-434).

Experiments are conducted in 5-10 week-old double transgenic rats (dTGRs). The model has been described in detail earlier. Briefly, the human renin construct are used to generate transgenic animals (hRen) made up the entire genomic human renin gene (10 exons and 9 introns), with 3.0 kB of the 5′-promoter region and 1.2 kB of 3′ additional sequences. The human angiotensinogen construct made up the entire human angiotensinogen gene (5 exons and 4 introns), with 1.3 kB of 5′-flanking and 2.4 kB of 3′-flanking sequences are used to generate rats producing human angiotensinogen (hAogen). The hRen and hAogen rats are rederived using embryo transfer from breeding pairs obtained under license from Ascencion Gmbh (Germany). The hAogen and hRen are then crossed to produce the double transgenic dTGR) off-spring. The dTGr rats are maintained on irradiated rodent chow (5VO2, Purina Mills Inc) and normal water. Radio telemetry transmitters (TA11PAC40, Data Sciences International) are surgically implanted at 5-6 weeks of age. The telemetry system provided 24-h recordings of systolic, mean, diastolic arterial pressure (SAP, MAP, DAP, respectively) and heart rate (HR). Prior to dosing, baseline hemodynamic measures are obtained for 24 hours. Rats are then dosed orally with vehicle or drug and monitored up to 48 hours post-dose.

While this invention has been particularly shown and described with references to preferred embodiments thereof, it will be understood by those skilled in the art that various changes in form and details may be made therein without departing from the scope of the invention encompassed by the appended claims.

1. A compound represented by the following structural formula: 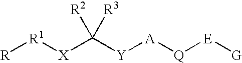
wherein:
R is:
a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)alkoxy, (C₃-C₈)alkenyloxy, (C₃-C₈)alkynyloxy, (C₃-C₇)cycloalkoxy, (C₅-C₇)cycloalkenyloxy, (C₃-C₇)cycloalkoxy(C₁-C₃)alkyl, (C₃-C₇)cycloalkyl(C₁-C₃)alkoxy, (C₅-C₇)cycloalkenyl(C₁-C₃)alkoxy, (C₁-C₈)alkylthio, (C₃-C₈)alkenylthio, (C₃-C₈)alkynylthio, (C₃-C₇)cycloalkylthio(C₁-C₃)alkyl, (C₃-C₇)cycloalkyl(C₁-C₃)alkylthio, (C₅-C₇)cycloalkenyl(C₁-C₃)alkylthio, (C₁-C₈)alkylamino, di(C₁-C₈)alkylamino, azepano, azetidino, piperidino, pyrrolidino, (C₃-C₇)cycloalkylamino, ((C₃-C₇)cycloalkyl(C₁-C₃)alkyl)amino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy and oxo;
b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, aryl(C₂-C₃))alkenyl, aryl(C₂-C₃)alkynyl, heteroaryl(C₂-C₃))alkenyl, or heteroaryl(C₂-C₃))alkynyl, each optionally substituted with up to three substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)-cycloalkyl(C₂-C₄)alkynyl, halo(C₁-C₆)alkyl, halo(C₃-C₆)cycloalkyl, halo(C₄-C₇)-cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkanesulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cyclo-alkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NCO, H₂NSO₂, (C₁-C₆)alkylaminocarbonyl, and di(C₁-C₆)alkylaminocarbonyl, (C₁-C₆)alkylaminosulfonyl, and di(C₁-C₆)alkylaminosulfonyl; or
c) a divalent radical selected from —(CH₂)₃—, —(CH₂)₄—, —(CH₂)₅— or —(CH₂)₆—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;
R¹ is phenyl, monocyclic heteroaryl, bicyclic heteroaryl, benzo-1,3-dioxole, benzo-1,3-dioxine, 2,3-dihydrobenzo-1,4-dioxine or (C₃-C₇)cycloalkyl, each optionally substituted with up to four substituents independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, H₂NSO₂, H₂NCO, (C₁-C₆)alkylaminosulfonyl, di(C₁-C₆)alkylaminosulfonyl, (C₁-C₆)alkylaminocarbonyl and di(C₁-C₆)alkylaminocarbonyl;
X and Y are each independently CH₂ or a single bond;
R² is
a) —H; or
b) (C₁-C₁₂)alkyl, (C₂-C₁₂)alkenyl, (C₂-C₁₂)alkynyl, (C₁-C₁₂)alkoxy, (C₁-C₁₂)alkylthio, (C₁-C₁₂)alkylamino, oxo(C₁-C₁₂)alkyl, oxo(C₂-C₁₂)alkenyl, oxo(C₂-C₁₂)alkynyl, oxo(C₁-C₁₂)alkoxy, oxo(C₁-C₁₂)alkylthio, oxo(C₁-C₁₂)alkylamino, (C₁-C₆)alkoxy(C₁-C₆)alkyl, (C₁-C₆)alkylthio(C₁-C₆)alkyl, (C₁-C₆)alkylamino(C₁-C₆)alkyl, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, (C₁-C₄)alkoxy(C₁-C₄)alkoxy(C₁-C₄)alkyl, aminocarbonylamino(C₁-C₁₂)alkyl, aminocarbonylamino(C₁-C₁₂)alkoxy, aminocarbonylamino(C₁-C₁₂)alkylthio, aminocarbonylamino(C₁-C₁₂)alkylamino, (C₁-C₆)-alkanoylamino(C₁-C₆)alkyl, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkyl, (C₁-C₆)alkoxycarbonyl(C₁-C₆)alkoxy, (C₁-C₆)alkoxycarbonyl(C₁-C₆)alkylthio, (C₁-C₆)alkoxy-carbonyl(C₁-C₆)alkylamino, (C₁-C₆)acyloxy(C₁-C₆)alkyl, (C₁-C₆)acyloxy(C₁-C₆)alkoxy, (C₁-C₆)acyloxy(C₁-C₆)alkylthio, (C₁-C₆)acyloxy(C₁-C₆)alkylamino, aminosulfonylamino(C₁-C₁₂)alkyl, aminosulfonylamino(C₁-C₁₂)alkoxy, aminosulfonylamino(C₁-C₁₂)alkylthio, aminosulfonyl-amino(C₁-C₁₂)alkylamino, (C₁-C₆)alkanesulfonylamino(C₁-C₆)alkyl, (C₁-C₆)alkanesulfonyl-amino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkyl, formylamino(C₁-C₆)alkoxy, formylamino(C₁-C₆)alkylthio, formylamino(C₁-C₆)alkylamino, (C₁-C₆)alkoxycarbonylamino(C₁-C₆)alkyl, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonylamino(C₁-C₆)alkyl, (C₁-C₆)alkylaminocarbonylamino(C₁-C₆)alkoxy, (C₁-C₆)alkylaminocarbonyl-amino(C₁-C₆)alkylthio, (C₁-C₆)alkylaminocarbonylamino(C₁-C₆)alkylamino, aminocarbonyl(C₁-C₆)alkyl, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkyl, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkyl, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkyl, (C₁-C₆)alkylamino-carboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by:
1) 1 to 5 halogen atoms; and
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, and halo(C₃-C₆)cycloalkoxy;
wherein the divalent sulfur atoms are independently optionally oxidized to sulfoxide or sulfone and wherein the carbonyl groups are optionally independently changed to a thiocarbonyl groups;
R³ is —H, halogen, (C₁-C₆)alkyl, (C₁-C₆)alkoxy, hydroxyl, hydroxy(C₁-C₆)alkyl, hydroxy(C₁-C₆)alkoxy, (C₁-C₆)alkanoylamino, (C₁-C₆)alkoxycarbonylamino, (C₁-C₆)alkylamino-carbonylamino, di(C₁-C₆)alkylaminocarbonylamino, (C₁-C₆)alkanesulfonylamino, (C₁-C₆)alkylaminosulfonylamino, di(C₁-C₆)alkylaminosulfonyl-amino, phenylamino or heteroarylamino in which each phenylamino or heteroarylamino group is optionally substituted with 1 to 5 groups independently selected from the group consisting of fluorine, chlorine, bromine, iodine, cyano, nitro, amino, hydroxy, carboxy, (C₁-C₆)alkyl, (C₃-C₆)cycloalkyl, (C₄-C₇)cycloalkylalkyl, (C₂-C₆)alkynyl, (C₃-C₆)cycloalkyl(C₂-C₄)alkynyl, halo(C₁-C₆)alkyl, halo(C₃-C₆)cycloalkyl, halo(C₄-C₇)cycloalkylalkyl, (C₁-C₆)alkoxy, (C₃-C₆)cycloalkoxy, (C₄-C₇)cycloalkylalkoxy, halo(C₁-C₆)alkoxy, halo(C₃-C₆)cycloalkoxy, halo(C₄-C₇)cycloalkylalkoxy, (C₁-C₆)alkylthio, (C₃-C₆)cycloalkylthio, (C₄-C₇)cycloalkylalkylthio, halo(C₁-C₆)alkylthio, halo(C₃-C₆)cycloalkylthio, halo(C₄-C₇)cycloalkylalkylthio, (C₁-C₆)alkanesulfinyl, (C₃-C₆)cycloalkanesulfinyl, (C₄-C₇)cycloalkylalkanesulfinyl, halo(C₁-C₆)alkane-sulfinyl, halo(C₃-C₆)cycloalkanesulfinyl, halo(C₄-C₇)-cycloalkylalkanesulfinyl, (C₁-C₆)alkanesulfonyl, (C₃-C₆)cycloalkanesulfonyl, (C₄-C₇)cycloalkylalkanesulfonyl, halo(C₁-C₆)alkanesulfonyl, halo(C₃-C₆)-cycloalkanesulfonyl, halo(C₄-C₇)cycloalkylalkanesulfonyl, (C₁-C₆)alkylamino, di(C₁-C₆)alkylamino, (C₁-C₆)alkoxy(C₁-C₆)alkoxy, halo(C₁-C₆)alkoxy(C₁-C₆)alkoxy, (C₁-C₆)alkoxycarbonyl, amino-carbonyl, (C₁-C₆)alkylaminocarbonyl, and di(C₁-C₆)alkylaminocarbonyl;
provided that
i) R² and R³ are not both hydrogen; and
ii) when R³ is hydroxyl, halogen, or optionally substituted phenylamino or heteroarylamino, R² is not (C₁-C₁₂)alkoxy, (C₁-C₁₂)alkylthio, (C₁-C₁₂)alkylamino, oxo(C₁-C₁₂)alkoxy, oxo(C₁-C₁₂)alkylthio, oxo(C₁-C₁₂)alkylamino, (C₁-C₆)alkoxy(C₁-C₆)alkoxy, (C₁-C₆)alkoxy(C₁-C₆)alkylthio, (C₁-C₆)alkoxy(C₁-C₆)alkylamino, (C₁-C₆)alkylthio(C₁-C₆)alkoxy, (C₁-C₆)alkylthio(C₁-C₆)alkylamino, (C₁-C₆)-alkylthio(C₁-C₆)alkylthio, (C₁-C₆)alkylamino(C₁-C₆)alkoxy, (C₁-C₆)alkylamino(C₁-C₆)alkylthio, (C₁-C₆)alkylamino(C₁-C₆)alkylamino, aminocarbonylamino(C₁-C₁₂)alkoxy, aminocarbonyl-amino(C₁-C₁₂)alkylthio, aminocarbonylamino(C₁-C₁₂)alkylamino, (C₁-C₆)alkanoylamino(C₁-C₆)alkoxy, (C₁-C₆)alkanoylamino(C₁-C₆)alkylthio, (C₁-C₆)alkanoylamino(C₁-C₆)alkylamino, (C₁-C₆)alkoxycarbonyl(C₁-C₆)alkoxy, (C₁-C₆)alkoxycarbonyl(C₁-C₆)alkylthio, (C₁-C₆)alkoxycarbonyl-(C₁-C₆)alkylamino, (C₁-C₆)acyloxy(C₁-C₆)alkoxy, (C₁-C₆)acyloxy(C₁-C₆)alkylthio, (C₁-C₆)-acyloxy(C₁-C₆)alkylamino, aminosulfonylamino(C₁-C₁₂)alkoxy, aminosulfonylamino(C₁-C₁₂)alkylthio, aminosulfonylamino(C₁-C₁₂)alkylamino, (C₁-C₆)alkanesulfonylamino(C₁-C₆)alkoxy, (C₁-C₆)alkanesulfonylamino(C₁-C₆)alkylthio, (C₁-C₆)alkanesulfonylamino(C₁-C₆)alkylamino, formylamino(C₁-C₆)alkoxy, formylamino(C₁-C₆)alkylthio, formylamino(C₁-C₆)alkylamino, (C₁-C₆)alkoxycarbonylamino(C₁-C₆)alkoxy, (C₁-C₆)alkoxycarbonylamino(C₁-C₆)alkylthio, (C₁-C₆)alkoxycarbonylamino(C₁-C₆)alkylamino, (C₁-C₆)alkylaminocarbonylamino(C₁-C₆)alkoxy, (C₁-C₆)alkylaminocarbonylamino(C₁-C₆)alkylthio, (C₁-C₆)alkylamino-carbonylamino(C₁-C₆)alkylamino, aminocarbonyl(C₁-C₆)alkoxy, aminocarbonyl(C₁-C₆)alkylthio, aminocarbonyl(C₁-C₆)alkylamino, (C₁-C₆)alkylaminocarbonyl(C₁-C₆)alkoxy, (C₁-C₆)alkylaminocarbonyl(C₁-C₆)alkylthio, (C₁-C₆)alkylaminocarbonyl(C₁-C₆)alkyamino, aminocarboxy(C₁-C₆)alkoxy, aminocarboxy(C₁-C₆)alkylthio, aminocarboxy(C₁-C₆)alkylamino, (C₁-C₆)alkylaminocarboxy(C₁-C₆)alkoxy, (C₁-C₆)alkylaminocarboxy(C₁-C₆)alkylthio, (C₁-C₆)alkylaminocarboxy(C₁-C₆)alkylamino, (C₁-C₁₂)alkoxycarbonylamino, (C₁-C₁₂)alkylamino-carbonylamino, or (C₁-C₁₂)alkanoylamino, each optionally substituted by
1) 1 to 5 halogen atoms; and
2) 1 group selected from cyano, hydroxyl, (C₁-C₃)alkyl, (C₁-C₃)alkoxy, (C₃-C₆)cycloalkyl, (C₃-C₆)cycloalkoxy, halo(C₁-C₃)alkyl, halo(C₁-C₃)alkoxy, halo(C₃-C₆)cycloalkyl, or halo(C₃-C₆)cycloalkoxy
wherein the divalent sulfur atoms are independently optionally oxidized to sulfoxide or sulfone and wherein the carbonyl groups are optionally independently changed to thiocarbonyl groups;
A is a saturated or unsaturated 4-, 5-, 6-, or 7-membered ring which is optionally bridged by (CH₂)ₘ via bonds to two members of said ring, wherein said ring is composed of carbon atoms and 0-2 hetero atoms selected from the group consisting of 0, 1, or 2 nitrogen atoms, 0 or 1 oxygen atoms, and 0 or 1 sulfur atoms, said ring being optionally substituted with up to four independently selected halogen atoms, (C₁-C₆)alkyl groups, halo(C₁-C₆)alkyl groups or oxo groups such that when there is substitution with one oxo group on a carbon atom it forms a carbonyl group and when there is substitution of one or two oxo groups on sulfur it forms sulfoxide or sulfone groups, respectively;
m is 1 to 3;
Q and Y are attached to carbon or nitrogen atoms in ring A in a 1, 2 or 1,3, or 1,4 relationship;
Q is a divalent radical selected from 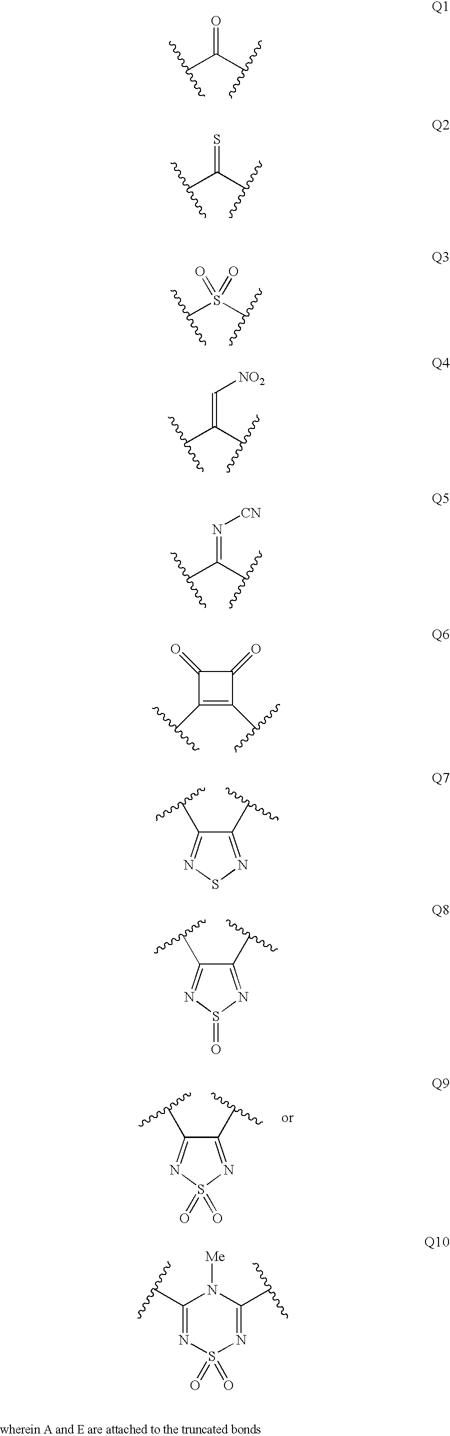
E is a saturated or unsaturated 3-, 4-, 5-, 6-, or 7-membered ring which is optionally bridged by (CH₂)ₙ via bonds to two members of said ring, wherein said ring is composed of carbon atoms, and 0-3 hetero atoms selected from 0, 1, 2. or 3 nitrogen atoms, 0 or 1 oxygen atoms, and 0 or 1 sulfur atoms, said ring being optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively;
n is 1 to 3;
G is hydroxy, hydroxy(C₁-C₆)alkyl, amino, (C₁-C₆)alkylamino, amino(C₁-C₆)alkyl, (C₁-C₆)alkylamino(C₁-C₆)alkyl, C(═NH)NH₂, C(═NH)NHR⁴, NHC(═NH)NH₂, or NHC(═NH)NHR⁴;
R⁴ is (C₁-C₃)alkyl;
or an enantiomer, diastereomer or salt thereof.

2. The compound of claim 1 wherein the compound is represented by the following structural formula: 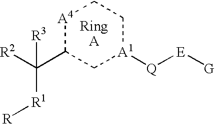
wherein:
R is
a) (C₁-C₈)alkyl, (C₂-C₈)alkenyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkyl(C₂-C₃)alkenyl, (C₃-C₇)cycloalkyl(C₂-C₃)alkynyl, (C₁-C₈)-alkoxy, (C₃-C₇)cycloalkoxy, (C₃-C₇)cycloalkoxy(C₁-C₃)alkyl, (C₃-C₇)cycloalkyl(C₁-C₃)alkoxy, (C₁-C₈)alkylthio, (C₃-C₇)cycloalkylthio, (C₃-C₇)cycloalkylthio(C₁-C₃)alkyl, (C₃-C₇)cycloalkyl(C₁-C₃)alkylthio, azepano, azetidino, piperidino, pyrrolidino or tri(C₁-C₄)alkylsilyl, each optionally substituted with up to four substituents independently selected from the group consisting of fluorine, hydroxy, (C₁-C₆)alkyl, halo(C₁-C₆)alkyl, (C₃-C₆)cycloalkyl, (C₁-C₆)alkoxy, (C₁-C₆)cycloalkoxy, and oxo;
b) aryl, heteroaryl, aryloxy, heteroaryloxy, aryl(C₁-C₃)alkyl, heteroaryl(C₁-C₃)alkyl, aryl(C₁-C₃)alkoxy, heteroaryl(C₁-C₃)alkoxy, arylethenyl, heteroarylethenyl, or arylethynyl, heteroarylethynyl, each optionally substituted with up to three substituents independently selected from the group consisting of fluorine, chlorine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NCO, H₂NSO₂, (C₁-C₆)alkylaminocarbonyl, and (C₁-C₆)alkylaminosulfonyl; or
c) a divalent radical selected from —(CH₂)₄— or —(CH₂)₅—, which is attached to R¹ to form a fused or spirofused ring system, and is optionally substituted with up to four substituents independently selected from the group consisting of fluorine, hydroxy, (C₁-C₆)alkyl, halo(C₁-C₆)alkyl, (C₁-C₆)alkoxy and oxo;
R¹ is a phenyl, monocyclic heteroaryl, bicyclic heteroaryl, benzo-1,3-dioxole, or (C₃-C₇)cycloalkyl ring optionally substituted with up to four substituents independently selected from the group consisting of fluorine, chlorine, bromine, cyano, (C₁-C₆)alkyl, (C₃-C₆)cycloalkyl, halo(C₁-C₆)alkyl, halo(C₃-C₆)cycloalkyl, (C₁-C₆)alkoxy, (C₃-C₆)cycloalkoxy, (C₄-C₇)cycloalkylalkoxy, halo(C₁-C₆)alkoxy, (C₁-C₆)alkylthio, halo(C₁-C₆)alkylthio, (C₁-C₆)alkanesulfinyl, halo(C₁-C₆)alkanesulfinyl, (C₁-C₆)alkanesulfonyl, halo(C₁-C₆)alkanesulfonyl, H₂NSO₂, H₂NCO, (C₁-C₃)alkylaminosulfonyl, and (C₁-C₃)alkylaminocarbonyl;
R² is
a) —H;
b) (C₁-C₁₀)alkyl, (C₂-C₁₀)alkenyl, (C₂-C₁₀)alkynyl, (C₁-C₁₀)alkoxy, (C₁-C₁₀)alkylthio, (C₁-C₁₀)alkylamino, (C₁-C₅)alkoxy(C₁-C₅)alkyl, (C₁-C₅)alkylthio(C₁-C₅)alkyl, (C₁-C₅)alkylamino(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₅)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, (C₁-C₃)alkoxy(C₁-C₃)alkoxy(C₁-C₃)alkyl, aminocarbonylamino(C₁-C₁₀)alkyl, aminocarbonylamino(C₁-C₁₀)alkoxy, aminocarbonylamino-(C₁-C₁₀)alkylthio, aminocarbonylamino(C₁-C₁₀)alkylamino, (C₁-C₅)alkanoylamino(C₁-C₅)alkyl, (C₁-C₅)alkanoylamino(C₁-C₅)alkoxy, (C₁-C₅)alkanoylamino(C₁-C₅)alkylthio, (C₁-C₅)-alkanoylamino(C₁-C₅)alkylamino, aminosulfonylamino(C₁-C₁₀)alkyl, aminosulfonylamino(C₁-C₁₀)alkoxy, aminosulfonylamino(C₁-C₁₀)alkylthio, aminosulfonylamino(C₁-C₁₀)alkylamino, (C₁-C₅)alkanesulfonylamino(C₁-C₅)alkyl, (C₁-C₅)alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkyl, formylamino(C₁-C₅)alkoxy, formylamino(C₁-C₅)alkylthio, formylamino(C₁-C₅)alkylamino, (C₁-C₅)alkoxycarbonylamino(C₁-C₅)alkyl, (C₁-C₅)alkoxy-carbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkyl, (C₁-C₅)alkylamino carbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkyl, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl(C₁-C₅)alkyl, (C₁-C₅)alkylaminocarbonyl(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkyl, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkyl, (C₁-C₅)alkylamino-carboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted by
1) 1 to 5 fluorine atoms; and
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy
wherein the divalent sulfur atoms are independently optionally oxidized to sulfoxide or sulfone;
R³ is —H, halogen, (C₁-C₃)alkyl, (C₁-C₃)alkoxy, hydroxyl, hydroxy(C₁-C₃)alkyl, hydroxy(C₁-C₃)alkoxy, (C₁-C₄)alkanoylamino, (C₁-C₃)alkoxycarbonylamino, (C₁-C₃)alkylamino-carbonylamino, di(C₁-C₃)alkylaminocarbonylamino, (C₁-C₃)alkanesulfonylamino, (C₁-C₃)alkylaminosulfonylamino, di(C₁-C₃)alkylaminosulfonylamino, or phenylamino or heteroarylamino in which each phenylamino and heteroarylamino group is optionally substituted with 1 to 3 groups independently selected from the group consisting of fluorine, chlorine, cyano, (C₁-C₃)alkyl, halo(C₁-C₃)alkyl, (C₁-C₃)alkoxy, halo(C₁-C₃)alkoxy, (C₁-C₃)alkanesulfonyl, and (C₁-C₃)alkoxycarbonyl;
provided that
i) R² and R³ are not both hydrogen and
ii) when R³ is hydroxyl, halogen, or optionally substituted phenylamino or heteroarylamino, R² is not (C₁-C₁₀)alkoxy, (C₁-C₁₀)alkylthio, (C₁-C₁₀)alkylamino, (C₁-C₅)alkylthio(C₁-C₅)alkyl, (C₁-C₅)alkoxy(C₁-C₅)alkoxy, (C₁-C₅)alkoxy(C₁-C₅)alkylthio, (C₁-C₅)alkoxy(C₁-C₅)alkylamino, (C₁-C₈)alkylthio(C₁-C₅)alkoxy, (C₁-C₅)alkylthio(C₁-C₅)alkylamino, (C₁-C₅)alkylthio(C₁-C₅)alkylthio, (C₁-C₅)alkylamino(C₁-C₅)alkoxy, (C₁-C₅)alkylamino(C₁-C₅)alkylthio, (C₁-C₅)alkylamino(C₁-C₅)alkylamino, aminocarbonylamino(C₁-C₁₀)alkoxy, aminocarbonylamino(C₁-C₁₀)alkylthio, aminocarbonyl-amino(C₁-C₁₀)alkylamino, (C₁-C₅)alkanoylamino(C₁-C₅)alkoxy, (C₁-C₅)alkanoylamino(C₁-C₅)alkylthio, (C₁-C₅)alkanoylamino(C₁-C₅)alkylamino, aminosulfonylamino(C₁-C₁₀)alkoxy, aminosulfonylamino(C₁-C₁₀)alkylthio, aminosulfonylamino(C₁-C₁₀)alkylamino, (C₁-C₅)-alkanesulfonylamino(C₁-C₅)alkoxy, (C₁-C₅)alkanesulfonylamino(C₁-C₅)alkylthio, (C₁-C₅)alkanesulfonylamino(C₁-C₅)alkylamino, formylamino(C₁-C₅)alkoxy, formylamino(C₁-C₅)alkylthio, formylamino(C₁-C₅)alkylamino, (C₁-C₅)alkoxycarbonylamino(C₁-C₅)alkoxy, (C₁-C₅)alkoxycarbonylamino(C₁-C₅)alkylthio, (C₁-C₅)alkoxycarbonylamino(C₁-C₅)alkylamino, (C₁-C₅)alkylaminocarbonylamino(C₁-C₅)alkoxy, (C₁-C₅)alkylaminocarbonylamino(C₁-C₅)alkylthio, (C₁-C₅)alkylaminocarbonylamino(C₁-C₅)alkylamino, aminocarbonyl(C₁-C₅)alkoxy, aminocarbonyl(C₁-C₅)alkylthio, aminocarbonyl(C₁-C₅)alkylamino, (C₁-C₅)alkylaminocarbonyl-(C₁-C₅)alkoxy, (C₁-C₅)alkylaminocarbonyl(C₁-C₅)alkylthio, (C₁-C₅)alkylaminocarbonyl(C₁-C₅)alkyamino, aminocarboxy(C₁-C₅)alkoxy, aminocarboxy(C₁-C₅)alkylthio, aminocarboxy(C₁-C₅)alkylamino, (C₁-C₅)alkylaminocarboxy(C₁-C₅)alkoxy, (C₁-C₅)alkylaminocarboxy(C₁-C₅)alkylthio, (C₁-C₅)alkylaminocarboxy(C₁-C₅)alkylamino, (C₁-C₁₀)alkoxycarbonylamino, (C₁-C₁₀)alkylaminocarbonylamino, or (C₁-C₁₀)alkanoylamino, each optionally substituted with
1) 1 to 5 fluorine atoms; and
2) 1 group selected from cyano, hydroxyl, (C₁-C₃)alkyl, (C₁-C₃)alkoxy, (C₃-C₄)cycloalkyl, (C₃-C₄)cycloalkoxy, halo(C₁-C₃)alkyl, halo(C₁-C₃)alkoxy, halo(C₃-C₄)cycloalkyl, and halo(C₃-C₄)cycloalkoxy
wherein the divalent sulfur atoms are independently optionally oxidized to sulfoxide or sulfone;
Ring A is
a) a benzene ring (A¹ and A⁴ are CH and the bonds in ring A are aromatic bonds);
b) piperidine, A¹ is N, A⁴ is CH₂ and the bonds in ring A are single bonds; or
c) morpholine, A¹ is N, A⁴ is 0 and the bonds in ring A are single bonds;
Q is a divalent radical selected from the group consisting of Q1, Q2, Q3, Q4, Q5, Q6, and Q7; 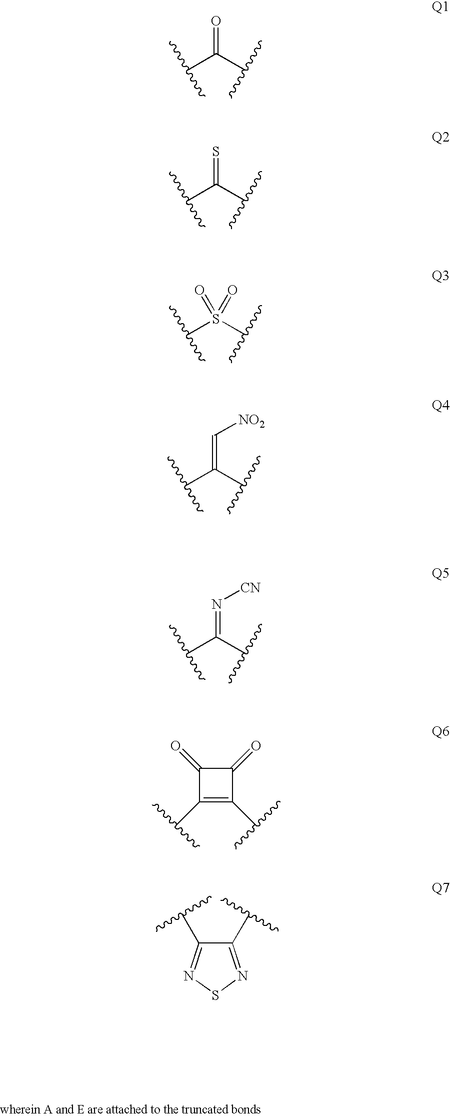
E is a saturated 3-, 4-, 5-, 6-, or 7-membered ring or an unsaturated 5- or 6-membered ring composed of carbon atoms and 0-3 hetero atoms selected from 0, 1, 2, or 3 nitrogen atoms, 0 or 1 oxygen atoms, and 0 or 1 sulfur atoms, said ring atoms being substituted with the appropriate number of hydrogen atoms, said ring being optionally substituted with up to four groups independently selected from halogen, hydroxy, (C₁-C₆)alkyl, halo(C₁-C₆)alkyl, hydroxy(C₁-C₆)alkyl, and oxo groups such that when there is substitution with one oxo group on a carbon atom it forms a carbonyl group and when there is substitution of one or two oxo groups on sulfur it forms sulfoxide or sulfone groups, respectively; and
G is hydroxy, hydroxy(C₁-C₃)alkyl, amino, (C₁-C₃)alkylamino, amino(C₁-C₃)alkyl, or (C₁-C₃)alkylamino(C₁-C₃)alkyl;
or an enantiomer, diastereomer, or salt thereof.

3. The compound of claim 2, wherein:
R is:
a) (C₁-C₈)alkyl, (C₂-C₈)alkynyl, (C₃-C₇)cycloalkyl, (C₅-C₇)cycloalkenyl, (C₃-C₇)cycloalkyl(C₁-C₃)alkyl, (C₃-C₇)cycloalkylethenyl, (C₃-C₇)cycloalkylethynyl, (C₁-C₈)alkoxy, (C₃-C₇)cycloalkoxy, (C₃-C₇)cycloalkoxy(C₁-C₃)alkyl, (C₃-C₇)cycloalkyl(C₁-C₃)alkoxy, piperidino, pyrrolidino or tri(C₁-C₃)alkylsilyl, each optionally substituted with up to 4 substituents independently selected from the group consisting of fluorine, hydroxy, (C₁-C₃)alkyl, and halo(C₁-C₃)alkyl,
b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to three substituents independently selected from the group consisting of halogen, cyano, (C₁-C₃)alkyl, (C₃-C₅)cycloalkyl, halo(C₁-C₃)alkyl, (C₁-C₃)alkoxy, halo(C₁-C₃)alkoxy, (C₁-C₃)alkylthio, and H₂NCO; or
c) a divalent radical selected from —(CH₂)₄— or —(CH₂)₅—, which is attached to R¹ to form a fused or spirofused ring system;
R¹ is a phenyl, monocyclic heteroaryl ring, bicyclic heteroaryl ring or benzo-1,3-dioxole, optionally substituted with up to four substituents independently selected from the group consisting of halogen, cyano, (C₁-C₃)alkyl, (C₃-C₄)cycloalkyl, halo(C₁-C₃)alkyl, (C₁-C₃)alkoxy, halo(C₁-C₃)alkoxy, and H₂NCO;
R² is —H, (C₁-C₈)alkyl, (C₄-C₉)cycloalkylalkyl, fluoro(C₁-C₈)alkyl, fluoro(C₄-C₈)-cycloalkylalkyl, (C₁-C₈)alkoxy, (C₄-C₉)cycloalkylalkoxy, fluoro(C₁-C₈)alkoxy, hydroxy(C₁-C₈)alkyl, (C₁-C₅)alkoxy(C₁-C₅)alkyl, halo(C₁-C₅)alkylamino(C₁-C₅)alkyl, (C₁-C₅)alkoxy(C₁-C₅)hydroxyalkyl, (C₃-C₄)cycloalkoxy(C₁-C₅)alkyl, fluoro(C₁-C₅)alkoxy(C₁-C₅)alkyl, fluoro(C₃-C₄)cycloalkoxy(C₁-C₅)alkyl, (C₁-C₅)alkylthio(C₁-C₅)alkyl, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₈)alkoxy, fluoro(C₃-C₄)cycloalkoxy(C₁-C₅)alkoxy, (C₁-C₃)alkoxy(C₁-C₃)alkoxy(C₁-C₃)alkyl, fluoro(C₁-C₃)alkoxy(C₁-C₃)alkoxy(C₁-C₃)alkyl, aminocarbonylamino(C₁-C₈)alkyl, aminocarbonylamino(C₁-C₈)alkoxy, (C₁-C₅)alkanoylamino(C₁-C₅)alkyl, (C₁-C₅)alkanoylamino(C₁-C₅)alkoxy, fluoro(C₁-C₅)alkanoylamino(C₁-C₅)alkyl, fluoro(C₁-C₅)alkanoylamino(C₁-C₅)alkoxy, (C₁-C₃)alkoxy(C₁-C₅)alkanoylamino(C₁-C₅)alkyl, (C₁-C₃)alkoxy(C₁-C₅)alkanoylamino(C₁-C₅)alkoxy, (C₃-C₄)-cycloalkanecarbonylamino(C₁-C₅)alkyl, (C₃-C₄)cycloalkanecarbonylamino(C₁-C₅)alkoxy, aminosulfonylamino(C₁-C₈)alkyl, aminosulfonylamino(C₁-C₈)alkoxy, (C₁-C₅)alkane-sulfonylamino(C₁-C₅)alkyl, (C₁-C₅)alkanesulfonylamino(C₁-C₅)alkoxy, formylamino(C₁-C₅)alkyl, formylamino(C₁-C₅)alkoxy, (C₁-C₅)alkoxycarbonylamino(C₁-C₅)alkyl, (C₁-C₅)alkoxycarbonylamino(C₁-C₅)alkoxy, (C₁-C₅)alkylaminocarbonylamino(C₁-C₅)alkyl, (C₁-C₅)alkylamino-carbonylamino(C₁-C₅)alkyl, di(C₁-C₅)alkylaminocarbonylamino(C₁-C₅)alkoxy, aminocarbonyl(C₁-C₅)alkyl, aminocarbonyl(C₁-C₅)alkoxy, (C₁-C₅)alkylaminocarbonyl(C₁-C₅)alkyl, (C₁-C₅)alkylaminocarbonyl(C₁-C₅)alkoxy, aminocarboxy(C₁-C₅)alkyl, aminocarboxy(C₁-C₅)alkoxy, (C₁-C₅)alkylaminocarboxy(C₁-C₅)alkyl, (C₁-C₅)alkylamino-carboxy(C₁-C₅)alkoxy, (C₁-C₈)alkoxycarbonylamino, (C₁-C₈)alkylaminocarbonylamino, (C₁-C₈)alkanoylamino, fluoro(C₁-C₈)alkoxycarbonylamino, fluoro(C₁-C₈)alkylaminocarbonylamino, or fluoro(C₁-C₈)alkanoylamino;
R³ is —H, halogen, OH, (C₁-C₄)alkanoylamino, or (C₁-C₃)alkoxy;
provided that
i) R² and R³ are not both hydrogen; and
ii) when R³ is OH or halogen, R² is not (C₁-C₈)alkoxy, (C₄-C₈)cycloalkylalkoxy, fluoro(C₁-C₈)alkoxy, (C₁-C₅)alkoxy(C₁-C₅)alkoxy, hydroxy(C₁-C₈)alkoxy, (C₃-C₄)cycloalkoxy(C₁-C₅)alkoxy, fluoro(C₁-C₅)alkoxy(C₁-C₅)alkoxy, fluoro(C₃-C₄)cycloalkoxy(C₁-C₅)alkoxy, aminocarbonylamino(C₁-C₈)alkoxy, (C₁-C₅)-alkanoylamino(C₁-C₅)alkoxy, fluoro(C₁-C₅)alkanoylamino(C₁-C₅)alkoxy, (C₁-C₃)alkoxy(C₁-C₅)alkanoylamino(C₁-C₅)alkoxy, (C₃-C₄)cycloalkanecarbonylamino(C₁-C₅)alkoxy, aminosulfonylamino(C₁-C₈)alkoxy, (C₁-C₅)alkanesulfonylamino(C₁-C₅)alkoxy, formylamino(C₁-C₅)alkoxy, (C₁-C₅)alkoxycarbonylamino(C₁-C₅)alkoxy, di(C₁-C₅)alkylaminocarbonylamino(C₁-C₅)alkoxy, aminocarbonyl(C₁-C₅)alkoxy, (C₁-C₅)alkylaminocarbonyl(C₁-C₅)alkoxy, aminocarboxy(C₁-C₅)alkoxy, (C₁-C₅)alkylaminocarboxy(C₁-C₅)alkoxy, (C₁-C₈)alkoxy-carbonylamino, (C₁-C₈)alkylaminocarbonylamino, (C₁-C₈)alkanoylamino, fluoro(C₁-C₈)alkoxy-carbonylamino, fluoro(C₁-C₈)alkylaminocarbonylamino, or fluoro(C₁-C₈)alkanoylamino;
Ring A is piperidine, morpholine or benzene;
Q is Q1, Q2, Q4, or Q6;
E is a saturated 3-, 4-, 5-, or 6-membered ring or an unsaturated 5- or 6-membered ring, wherein said ring is composed of carbon atoms, and 0-3 hetero atoms selected from 0, 1, 2, or 3 nitrogen atoms, 0 or 1 oxygen atoms, and 0 or 1 sulfur atoms, said ring being optionally substituted with up to four groups independently selected from fluorine, hydroxy, (C₁-C₃)alkyl, hydroxy(C₁-C₃)alkyl, and oxo groups such that when there is substitution with one oxo group on a carbon atom it forms a carbonyl group and when there is substitution of one or two oxo groups on sulfur it forms sulfoxide or sulfone groups, respectively; and
G is amino, (C₁-C₃)alkylamino, amino(C₁-C₃)alkyl, or (C₁-C₃)alkylamino(C₁-C₃)alkyl.

4. The compound of claim 3, wherein
R is:
a) (C₁-C₇)alkyl, (C₃-C₇)cycloalkyl, (C₅-C₇)cycloalkenyl, (C₁-C₇)alkoxy, (C₃-C₇)cycloalkoxy, (C₃-C₇)cycloalkyl(C₁-C₃)alkoxy, piperidino, pyrrolidino or tri(C₁-C₃)alkylsilyl, each optionally substituted with up to 4 substituents independently selected from fluorine, hydroxy, (C₁-C₃)alkyl, or halo(C₁-C₃)alkyl; or
b) phenyl, monocyclic heteroaryl, phenoxy, monocyclic heteroaryloxy, phenyl(C₁-C₃)alkoxy, or monocyclic heteroaryl(C₁-C₃)alkoxy, each optionally substituted with up to 3 substituents independently selected from fluorine, chlorine, cyano, (C₁-C₃)alkyl, (C₃-C₄)cycloalkyl, halo(C₁-C₃)alkyl, (C₁-C₃)alkoxy, (C₁-C₃)alkylthio or H₂NCO; or
c) —(CH₂)₄— or —(CH₂)₅—;
R¹ is a phenyl, furan, thiophene, pyrrole, pyrazole, imidazole, oxazole, thiazole, pyridine, pyrimidine, pyrazine, benzofuran, benzothiophene, benzoxazole, benzothiazole, benzimidazole, quinoline, isoquinoline, quinazoline or benzo-1,3-dioxole, each optionally substituted with up to 3 substituents independently selected from the group consisting of fluorine, chlorine, cyano, (C₁-C₃)alkyl, halo(C₁-C₃)alkyl, (C₁-C₃)alkoxy, and carboxamide;
R² is (C₁-C₃)alkoxy(C₁-C₅)alkyl, (C₁-C₃)alkoxy(C₁-C₅)alkoxy, (C₃-C₄)cycloalkyl(C₁-C₅)alkyl, (C₃-C₄)cycloalkyl(C₁-C₅)alkoxy, (C₁-C₃)alkoxycarbonylamino(C₁-C₅)alkyl, (C₁-C₃)-alkoxycarbonylamino(C₁-C₅)alkoxy, (C₁-C₃)alkanoylamino(C₁-C₅)alkyl, (C₁-C₃)-alkanoylamino(C₁-C₅)alkoxy, (C₁-C₃)alkylaminocarbonyl(C₁-C₅)alkyl or (C₁-C₃)alkylaminocarbonyl(C₁-C₅)alkoxy;
R³ is hydrogen, fluoro, hydroxyl, or (C₁-C₄)alkanoylamino, provided that when R³ is hydroxyl or fluoro, R² is not (C₁-C₃)alkoxy(C₁-C₅)alkoxy, (C₃-C₄)cycloalkyl(C₁-C₅)alkoxy, (C₁-C₃)alkoxy-carbonylamino(C₁-C₅)alkoxy, (C₁-C₃)alkanoylamino(C₁-C₅)alkoxy or (C₁-C₃)alkylaminocarbonyl(C₁-C₅)alkoxy;
Ring A is piperidine, morpholine, or benzene
Q is Q1, Q2, Q4 or Q6;
E is a saturated 3-, 4-, 5-, or 6-membered ring or an unsaturated 5- or 6-membered ring composed of carbon atoms and 0 or 1 nitrogen atoms, said ring being optionally substituted with up to one hydroxy or hydroxy(C₁-C₃)alkyl group and with up to two (C₁-C₃)alkyl groups; and
G is amino, amino(C₁-C₃)alkyl, (C₁-C₃)alkylamino, or (C₁-C₃)alkylamino(C₁-C₃)alkyl.

5. The compound of claim 4, wherein
R is ethyl, isobutyl, t-butyl, 2,2-dimethyl-1-propoxy, cyclopentyloxy, cyclopropylmethoxy, 2-(cyclopropyl)ethoxy, cyclobutylmethoxy, cyclopentylmethoxy, cyclohexylmethoxy, benzyloxy, 4-fluorobenzyloxy, phenyl, 2-fluorophenyl, 2-chlorophenyl, 2-methylphenyl, 3-fluorophenyl, 3-chlorophenyl, 3-methylphenyl, 3-ethylphenyl, 3-isopropylphenyl, 3-cyclopropylphenyl, 3-methoxyphenyl, 3-ethoxyphenyl, 3-(methylthio)phenyl, 3-(trifluoromethyl)phenyl, 4-fluorophenyl, 4-chlorophenyl, 4-methylphenyl, 2,3-difluorophenyl, 2-fluoro-3-chlorophenyl, 2-fluoro-5-methylphenyl, 3,4-difluorophenyl, 3,4-dimethylphenyl, 3,5-dimethylphenyl, 5-methyl-2-furyl, 2-pyridyl, 1-cyclohexenyl, phenoxy, 2-fluorophenoxy, 2-chlorophenoxy, 2-methylphenoxy, 2-ethylphenoxy, 3-fluorophenoxy, 3-methylphenoxy, 4-fluorophenoxy, 4-methylphenoxy, 2-methyl-4-fluorophenoxy, 2-methyl-5-fluorophenoxy, piperidino, trimethylsilyl, —(CH₂)₄— or —(CH₂)₅—;
R¹ is phenyl, 2-fluorophenyl, 3-fluorophenyl, 3-chlorophenyl, 3-methylphenyl, 4-fluorophenyl, 4-cyanophenyl, 5-fluorophenyl, 6-fluorophenyl, 6-methoxyphenyl, 3,5-difluorophenyl, benzofuran, benzothiophene, benzooxazole or benzo-1,3-dioxole;
R² is 4-methoxybutyl, 4-ethoxybutyl, 4-methoxypentyl, 3-methoxypropoxy, 3-(methoxycarbonylamino)propyl, 3-(acetylamino)propyl, 2-(acetylamino)ethoxy, or 2-(methoxycarbonylamino)ethoxy;
R³ is hydrogen or hydroxyl provided that when R³ is hydroxyl, R² is not 3-methoxypropoxy, 2-(acetylamino)ethoxy, or 2-(methoxycarbonylamino)ethoxy;
Ring A is piperidine, morpholine, or benzene;
Q is Q1, Q4, or Q6;
E is azetidine, pyrrolidine, hydroxypyrrolidine, (hydroxymethyl)pyrrolidine, methylpyrrolidine, piperidine, hydroxypiperidine, cyclopropane, methylcyclopropane, cyclopentane, hydroxycyclopentane, cyclohexane, hydroxycyclohexane, or pyridine; and
G is amino, aminomethyl, methylamino or methylaminomethyl.

6. The compound of claim 2, wherein the compound is represented by the following structural formula: 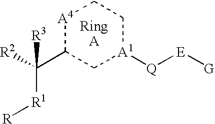
or an enantiomer, diastereomer, or salt thereof.

7. The compound of claim 2, wherein the compound is represented by the following structural formula: 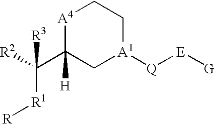
or an enantiomer, diastereomer, or salt thereof.

8. The compound of claim 2, wherein the compound is represented by the following structural formula: 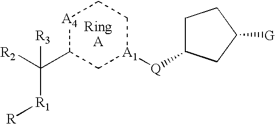
or an enantiomer, diastereomer, or salt thereof.

9. The compound of claim 2, wherein the compound is represented by the following structural formula: 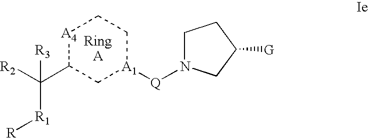
or an enantiomer, diastereomer, or salt thereof.

10. A pharmaceutical composition comprising a compound of claim 1, or an enantiomer, diastereomer, or salt thereof and a pharmaceutically acceptable carrier or excipient.

11. The pharmaceutical composition of claim 10, further comprising an additional agent selected from the group consisting of α-blockers, γ-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

12. A method of antagonizing aspartic protease inhibitors in a subject in need thereof comprising administering to the subject a therapeutically effective amount of a compound claim 1 or an enantiomer, diastereomer, or salt thereof.

13. The method of claim 12, wherein the aspartic protease is renin.

14. A method for treating or ameliorating an aspartic protease mediated disorder in a subject in need thereof comprising administering to said subject a therapeutically effective amount of a compound of claim 1, or an enantiomer, diastereomer, or salt thereof.

15. The method of claim 14, wherein the aspartic protease is β-secretase.

16. The method of claim 14, wherein the aspartic protease is plasmepsin.

17. The method of claim 14, wherein the aspartic protease is HIV protease.

18. A method for treating or ameliorating a renin mediated disorder in a subject in need thereof comprising administering to the subject an effective amount of a compound of claim 1, or an enantiomer, diastereomer, or salt thereof.

19. The method of claim 18, wherein the renin mediated disorder is hypertension, congestive heart failure, cardiac hypertrophy, cardiac fibrosis, cardiomyopathy post-infarction, complications resulting from diabetes, such as nephropathy, vasculopathy and neuropathy, diseases of the coronary vessels, post-surgical hypertension, restenosis following angioplasty, raised intra-ocular pressure, glaucoma, abnormal vascular growth, hyperaldosteronism, anxiety states, or a cognitive disorder.

20. A method for the treatment of hypertension in a subject in need thereof comprising administering to the subject a compound of claim 1 in combination therapy with one or more additional agents said additional agent selected from the group consisting of α-blockers, β-blockers, calcium channel blockers, diuretics, angiotensin converting enzyme (ACE) inhibitors, dual ACE and neutral endopeptidase (NEP) inhibitors, angiotensin-receptor blockers (ARBs), aldosterone synthase inhibitors, aldosterone-receptor antagonists, and endothelin receptor antagonists.

21. The method of claim 20, wherein:
the α-blockers are selected from the group consisting of doxazosin, prazosin, tamsulosin, and terazosin;
the β-blockers are selected from the group consisting of atenolol, bisoprol, metoprolol, acetutolol, esmolol, celiprolol, taliprolol, acebutolol, oxprenolol, pindolol, propanolol, bupranolol, penbutolol, mepindolol, carteolol, nadolol, and carvedilol, or pharmaceutically acceptable salts thereof;
the calcium channel blockers are selected from the group consisting of dihydropyridines (DHPs) and non-DHPs, wherein the DHPs are selected from the group consisting of amlodipine, felodipine, ryosidine, isradipine, lacidipine, nicardipine, nifedipine, nigulpidine, modiphine, nisoldipine, nitrendipine, and nivaldipine and their pharmaceutically acceptable salts and the non-DHPs are selected from the group consisting of flunarizine, prenylamine, diltiazem, fendiline, gallopamil, mibefradil, anipamil, tiapamil, and verampimil, or pharmaceutically acceptable salts thereof;
the diuretics is a thiazide derivative selected from the group consisting of an amiloride, chlorothiazide, hydrochlorothiazide, methylchlorothiazide, and chlorothalidon;
the ACE inhibitors are selected from the group consisting of alacepril, benazepril, benazaprilat, captopril, ceronapril, cilazapril, delapril, enalapril, enalaprilat, fosinopril, lisinopril, moexipiril, moveltopril, perindopril, quinapril, quinaprilat, ramipril, ramiprilat, spirapril, temocapril, trandolapril, and zofenopril;
dual ACE/NEP are selected from the group consisting of include omapatrilat, fasidotril, and fasidotrilat;
the ARBs are selected from the group consisting of candesartan, eprosartan, irbesartan, losartan, olmesartan, tasosartan, telmisartan, and valsartan;
the aldosterone synthase inhibitors are selected from the group consisting of anastrozole, fadrozole, and exemestane;
the aldosterone-receptor antagonists are selected from the group consisting of spironolactone and eplerenone; and
the endothelin antagonists are selected from the group consisting of bosentan, enrasentan, atrasentan, darusentan, sitaxentan, and tezosentan, or pharmaceutically acceptable salts thereof.

22. The method of claim 21, wherein the compound and the additional agents are administered by sequential administration or simultaneous administration.

23. (canceled)
